(19) United States
(12) Patent Application Publication
(10) Pub. No.: US 2003/0166114 A1
(43) Pub. Date: Sep. 4, 2003

(54) Secreted and transmembrane polypeptides and nucleic acids encoding the same

(75) Inventors: Kevin P. Baker, Darnestown, MD (US); Jian Chen, Princeton, NJ (US); Luc Desnoyers, San Francisco, CA (US); Audrey Goddard, San Francisco, CA (US); Paul J. Godowski, Hillsborough, CA (US); Austin L. Gurney, Belmont, CA (US); James Pan, Belmont, CA (US); Victoria Smith, Burlingame, CA (US); Colin K. Watanabe, Moraga, CA (US); William I. Wood, Hillsborough, CA (US); Zemin Zhang, Foster City, CA (US)

Correspondence Address: KNOBBE, MARTENS, OLSON & BEAR, LLP, 2040 MAIN STREET, FOURTEENTH FLOOR, IRVINE, CA, 92614, US

(73) Assignee: Genentech, Inc.

(21) Appl. No.: 10/174,589

(22) Filed: Jun. 18, 2002

Related U.S. Application Data
(63) Continuation of application No. 10/052,586, filed on Jan. 15, 2002.
(63) Continuation of application No. 09/168,978, filed on Oct. 7, 1998, now abandoned.
(63) Continuation of application No. 09/187,368, filed on Nov. 6, 1998, now abandoned.
(63) Continuation of application No. 09/202,054, filed on Dec. 7, 1998.
(63) Continuation of application No. 09/254,311, filed on Mar. 3, 1999, now abandoned.
(63) Continuation of application No. 09/311,832, filed on May 14, 1999.
(63) Continuation of application No. 09/380,137.
(63) Continuation of application No. 09/380,138, filed on Aug. 25, 1999, now abandoned.
(63) Continuation of application No. 09/380,139, filed on Aug. 25, 1999, now abandoned.
(63) Continuation of application No. 09/380,142, filed on Aug. 25, 1999, now abandoned.
(63) Continuation of application No. 09/403,297, filed on Oct. 18, 1999, now abandoned.
(63) Continuation of application No. 09/423,844, filed on Nov. 12, 1999, now abandoned.
(63) Continuation of application No. 09/644,848, filed on Aug. 22, 2000.
(63) Continuation of application No. 09/664,610, filed on Sep. 18, 2000, now abandoned.
(63) Continuation of application No. 09/665,350, filed on Sep. 18, 2000.
(63) Continuation of application No. 09/709,238, filed on Nov. 8, 2000, now abandoned.
(63) Continuation of application No. 09/747,259, filed on Dec. 20, 2000, now Pat. No. 6,569,645.
(63) Continuation of application No. 09/816,744, filed on Mar. 22, 2001.
(63) Continuation of application No. 09/854,208, filed on May 10, 2001.
(63) Continuation of application No. 09/854,280, filed on May 10, 2001.
(63) Continuation of application No. 09/866,028, filed on May 25, 2001.
(63) Continuation of application No. 09/874,503, filed on Jun. 5, 2001.
(63) Continuation of application No. 09/908,827, filed on Jul. 18, 2001.
(63) Continuation of application No. 09/918,585, filed on Jul. 30, 2001.
(63) Continuation of application No. 09/924,419, filed on Aug. 6, 2001, now abandoned.
(63) Continuation of application No. 09/929,404, filed on Aug. 13, 2001, now abandoned.
(63) Continuation of application No. 09/931,836, filed on Aug. 16, 2001.
(63) Continuation of application No. 09/941,992, filed on Aug. 28, 2001.
(63) Continuation of application No. 09/946,374, filed on Sep. 4, 2001.
(60) Provisional application No. 60/059,263, filed on Sep. 18, 1997.
(60) Provisional application No. 60/059,266, filed on Sep. 18, 1997.
(60) Provisional application No. 60/062,250, filed on Oct. 17, 1997.
(60) Provisional application No. 60/063,120, filed on Oct. 24, 1997.
(60) Provisional application No. 60/063,121, filed on Oct. 24, 1997.
(60) Provisional application No. 60/063,486, filed on Oct. 21, 1997.
(60) Provisional application No. 60/063,540, filed on Oct. 28, 1997.
(60) Provisional application No. 60/063,541, filed on Oct. 28, 1997.
(60) Provisional application No. 60/063,544, filed on Oct. 28, 1997.
(60) Provisional application No. 60/063,564, filed on Oct. 28, 1997.
(60) Provisional application No. 60/063,734, filed on Oct. 29, 1997.
(60) Provisional application No. 60/063,870, filed on Oct. 31, 1997.
(60) Provisional application No. 60/064,103, filed on Oct. 31, 1997.
(60) Provisional application No. 60/065,311, filed on Nov. 13, 1997.
(60) Provisional application No. 60/066,120, filed on Nov. 21, 1997.
(60) Provisional application No. 60/066,466, filed on Nov. 24, 1997.
(60) Provisional application No. 60/066,772, filed on Nov. 24, 1997.
(60) Provisional application No. 60/068,017, filed on Dec. 18, 1997.
(60) Provisional application No. 60/069,335, filed on Dec. 11, 1997.
(60) Provisional application No. 60/069,425, filed on Dec. 12, 1997.
(60) Provisional application No. 60/069,870, filed on Dec. 17, 1997.
(60) Provisional application No. 60/077,450, filed on Mar. 10, 1998.
(60) Provisional application No. 60/077,632, filed on Mar. 11, 1998.
(60) Provisional application No. 60/077,649, filed on Mar. 11, 1998.
(60) Provisional application No. 60/078,886, filed on Mar. 20, 1998.
(60) Provisional application No. 60/078,939, filed on Mar. 20, 1998.
(60) Provisional application No. 60/079,664, filed on Mar. 27, 1998.
(60) Provisional application No. 60/079,786, filed on Mar. 27, 1998.
(60) Provisional application No. 60/080,107, filed on Mar. 31, 1998.
(60) Provisional application No. 60/080,194, filed on Mar. 31, 1998.
(60) Provisional application No. 60/080,327, filed on Apr. 1, 1998.
(60) Provisional application No. 60/080,333, filed on Apr. 1, 1998.
(60) Provisional application No. 60/081,049, filed on Apr. 8, 1998.
(60) Provisional application No. 60/081,070, filed on Apr. 8, 1998.
(60) Provisional application No. 60/081,195, filed on Apr. 9, 1998.
(60) Provisional application No. 60/081,838, filed on Apr. 15, 1998.
(60) Provisional application No. 60/082,568, filed on Apr. 21, 1998.
(60) Provisional application No. 60/082,569, filed on Apr. 21, 1998.
(60) Provisional application No. 60/082,704, filed on Apr. 22, 1998.
(60) Provisional application No. 60/082,797, filed on Apr. 22, 1998.
(60) Provisional application No. 60/083,322, filed on Apr. 28, 1998.
(60) Provisional application No. 60/083,495, filed on Apr. 29, 1998.
(60) Provisional application No. 60/083,496, filed on Apr. 29, 1998.
(60) Provisional application No. 60/083,499, filed on Apr. 29, 1998.
(60) Provisional application No. 60/083,559, filed on Apr. 29, 1998.
(60) Provisional application No. 60/084,366, filed on May 5, 1998.
(60) Provisional application No. 60/084,414, filed on May 6, 1998.
(60) Provisional application No. 60/084,639, filed on May 7, 1998.
(60) Provisional application No. 60/084,640, filed on May 7, 1998.
(60) Provisional application No. 60/084,643, filed on May 7, 1998.
(60) Provisional application No. 60/085,579, filed on May 15, 1998.
(60) Provisional application No. 60/085,580, filed on May 15, 1998.
(60) Provisional application No. 60/085,582, filed on May 15, 1998.
(60) Provisional application No. 60/085,700, filed on May 15, 1998.
(60) Provisional application No. 60/086,023, filed on May 18, 1998.
(60) Provisional application No. 60/086,392, filed on May 22, 1998.
(60) Provisional application No. 60/086,486, filed on May 22, 1998.
(60) Provisional application No. 60/087,098, filed on May 28, 1998.
(60) Provisional application No. 60/087,208, filed on May 28, 1998.
(60) Provisional application No. 60/087,609, filed on Jun. 2, 1998.
(60) Provisional application No. 60/087,759, filed on Jun. 2, 1998.
(60) Provisional application No. 60/087,827, filed on Jun. 3, 1998.
(60) Provisional application No. 60/088,025, filed on Jun. 4, 1998.
(60) Provisional application No. 60/088,028, filed on Jun. 4, 1998.
(60) Provisional application No. 60/088,029, filed on Jun. 4, 1998.
(60) Provisional application No. 60/088,033, filed on Jun. 4, 1998.
(60) Provisional application No. 60/088,167, filed on Jun. 5, 1998.
(60) Provisional application No. 60/088,202, filed on Jun. 5, 1998.
(60) Provisional application No. 60/088,212, filed on Jun. 5, 1998.
(60) Provisional application No. 60/088,217, filed on Jun. 5, 1998.
(60) Provisional application No. 60/088,326, filed on Jun. 4, 1998.
(60) Provisional application No. 60/088,655, filed on Jun. 9, 1998.
(60) Provisional application No. 60/088,722, filed on Jun. 10, 1998.
(60) Provisional application No. 60/088,738, filed on Jun. 10, 1998.
(60) Provisional application No. 60/088,740, filed on Jun. 10, 1998.
(60) Provisional application No. 60/088,811, filed on Jun. 10, 1998.
(60) Provisional application No. 60/088,824, filed on Jun. 10, 1998.
(60) Provisional application No. 60/088,825, filed on Jun. 10, 1998.
(60) Provisional application No. 60/088,826, filed on Jun. 10, 1998.
(60) Provisional application No. 60/088,861, filed on Jun. 11, 1998.
(60) Provisional application No. 60/088,863, filed on Jun. 11, 1998.
(60) Provisional application No. 60/088,876, filed on Jun. 11, 1998.
(60) Provisional application No. 60/089,090, filed on Jun. 12, 1998.
(60) Provisional application No. 60/089,105, filed on Jun. 12, 1998.
(60) Provisional application No. 60/089,512, filed on Jun. 16, 1998.
(60) Provisional application No. 60/089,514, filed on Jun. 16, 1998.
(60) Provisional application No. 60/089,538, filed on Jun. 17, 1998.
(60) Provisional application No. 60/089,598, filed on Jun. 17, 1998.
(60) Provisional application No. 60/089,653, filed on Jun. 17, 1998.
(60) Provisional application No. 60/089,908, filed on Jun. 18, 1998.
(60) Provisional application No. 60/089,952, filed on Jun. 19, 1998.
(60) Provisional application No. 60/090,246, filed on Jun. 22, 1998.
(60) Provisional application No. 60/090,252, filed on Jun. 22, 1998.
(60) Provisional application No. 60/090,254, filed on Jun. 22, 1998.
(60) Provisional application No. 60/090,429, filed on Jun. 24, 1998.
(60) Provisional application No. 60/090,435, filed on Jun. 24, 1998.
(60) Provisional application No. 60/090,444, filed on Jun. 24, 1998.
(60) Provisional application No. 60/090,461, filed on Jun. 24, 1998.
(60) Provisional application No. 60/090,535, filed on Jun. 24, 1998.
(60) Provisional application No. 60/090,540, filed on Jun. 24, 1998.
(60) Provisional application No. 60/090,676, filed on Jun. 25, 1998.
(60) Provisional application No. 60/090,678, filed on Jun. 25, 1998.
(60) Provisional application No. 60/090,688, filed on Jun. 25, 1998.
(60) Provisional application No. 60/090,690, filed on Jun. 25, 1998.
(60) Provisional application No. 60/090,694, filed on Jun. 25, 1998.
(60) Provisional application No. 60/090,695, filed on Jun. 25, 1998.
(60) Provisional application No. 60/090,696, filed on Jun. 25, 1998.
(60) Provisional application No. 60/090,862, filed on Jun. 26, 1998.
(60) Provisional application No. 60/090,863, filed on Jun. 26, 1998.
(60) Provisional application No. 60/091,010, filed on Jun. 26, 1998.
(60) Provisional application No. 60/091,359, filed on Jul. 1, 1998.
(60) Provisional application No. 60/091,478, filed on Jul. 2, 1998.
(60) Provisional application No. 60/091,486, filed on Jul. 2, 1998.
(60) Provisional application No. 60/091,544, filed on Jul. 1, 1998.
(60) Provisional application No. 60/091,626, filed on Jul. 2, 1998.
(60) Provisional application No. 60/091,628, filed on Jul. 2, 1998.
(60) Provisional application No. 60/091,632, filed on Jul. 2, 1998.
(60) Provisional application No. 60/094,006, filed on Jul. 24, 1998.
(60) Provisional application No. 60/095,282, filed on Aug. 4, 1998.
(60) Provisional application No. 60/095,998, filed on Aug. 10, 1998.
(60) Provisional application No. 60/096,012, filed on Aug. 10, 1998.
(60) Provisional application No. 60/096,757, filed on Aug. 17, 1998.
(60) Provisional application No. 60/096,766, filed on Aug. 17, 1998.
(60) Provisional application No. 60/096,867, filed on Aug. 17, 1998.
(60) Provisional application No. 60/096,891, filed on Aug. 17, 1998.
(60) Provisional application No. 60/096,897, filed on Aug. 17, 1998.
(60) Provisional application No. 60/096,949, filed on Aug. 18, 1998.
(60) Provisional application No. 60/096,959, filed on Aug. 18, 1998.
(60) Provisional application No. 60/097,022, filed on Aug. 18, 1998.
(60) Provisional application No. 60/097,952, filed on Aug. 26, 1998.
(60) Provisional application No. 60/097,954, filed on Aug. 26, 1998.
(60) Provisional application No. 60/097,955, filed on Aug. 26, 1998.
(60) Provisional application No. 60/097,971, filed on Aug. 26, 1998.
(60) Provisional application No. 60/097,974, filed on Aug. 26, 1998.
(60) Provisional application No. 60/098,014, filed on Aug. 26, 1998.
(60) Provisional application No. 60/098,716, filed on Sep. 1, 1998.
(60) Provisional application No. 60/098,723, filed on Sep. 1, 1998.
(60) Provisional application No. 60/098,803, filed on Sep. 2, 1998.
(60) Provisional application No. 60/098,821, filed on Sep. 2, 1998.
(60) Provisional application No. 60/098,843, filed on Sep. 2, 1998.
(60) Provisional application No. 60/099,602, filed on Sep. 9, 1998.
(60) Provisional application No. 60/099,741, filed on Sep. 10, 1998.
(60) Provisional application No. 60/099,754, filed on Sep. 10, 1998.
(60) Provisional application No. 60/099,763, filed on Sep. 10, 1998.
(60) Provisional application No. 60/099,812, filed on Sep. 10, 1998.
(60) Provisional application No. 60/100,388, filed on Sep. 15, 1998.
(60) Provisional application No. 60/100,662, filed on Sep. 16, 1998.
(60) Provisional application No. 60/100,664, filed on Sep. 16, 1998.
(60) Provisional application No. 60/100,683, filed on Sep. 17, 1998.
(60) Provisional application No. 60/100,684, filed on Sep. 17, 1998.
(60) Provisional application No. 60/100,849, filed on Sep. 18, 1998.
(60) Provisional application No. 60/100,919, filed on Sep. 17, 1998.
(60) Provisional application No. 60/100,930, filed on Sep. 17, 1998.
(60) Provisional application No. 60/101,014, filed on Sep. 18, 1998.
(60) Provisional application No. 60/101,068, filed on Sep. 18, 1998.
(60) Provisional application No. 60/101,471, filed on Sep. 23, 1998.
(60) Provisional application No. 60/101,472, filed on Sep. 23, 1998.
(60) Provisional application No. 60/101,475, filed on Sep. 23, 1998.
(60) Provisional application No. 60/101,477, filed on Sep. 23, 1998.
(60) Provisional application No. 60/101,738, filed on Sep. 24, 1998.
(60) Provisional application No. 60/101,739, filed on Sep. 24, 1998.
(60) Provisional application No. 60/101,743, filed on Sep. 24, 1998.
(60) Provisional application No. 60/101,751, filed on Sep. 16, 1998.
(60) Provisional application No. 60/101,786, filed on Sep. 25, 1998.
(60) Provisional application No. 60/101,922, filed on Sep. 24, 1998.
(60) Provisional application No. 60/102,207, filed on Sep. 29, 1998.
(60) Provisional application No. 60/102,240, filed on Sep. 29, 1998.
(60) Provisional application No. 60/102,330, filed on Sep. 29, 1998.
(60) Provisional application No. 60/102,331, filed on Sep. 29, 1998.
(60) Provisional application No. 60/102,487, filed on Sep. 30, 1998.
(60) Provisional application No. 60/102,570, filed on Sep. 30, 1998.
(60) Provisional application No. 60/102,571, filed on Sep. 30, 1998.
(60) Provisional application No. 60/102,684, filed on Oct. 1, 1998.
(60) Provisional application No. 60/102,687, filed on Oct. 1, 1998.
(60) Provisional application No. 60/102,965, filed on Oct. 2, 1998.
(60) Provisional application No. 60/103,258, filed on Oct. 6, 1998.
(60) Provisional application No. 60/103,395, filed on Oct. 7, 1998.
(60) Provisional application No. 60/103,401, filed on Oct. 7, 1998.
(60) Provisional application No. 60/103,449, filed on Oct. 6, 1998.
(60) Provisional application No. 60/103,633, filed on Oct. 8, 1998.
(60) Provisional application No. 60/103,678, filed on Oct. 8, 1998.
(60) Provisional application No. 60/103,679, filed on Oct. 8, 1998.
(60) Provisional application No. 60/103,681, filed on Oct. 8, 1998.
(60) Provisional application No. 60/103,711, filed on Oct. 8, 1998.
(60) Provisional application No. 60/104,257, filed on Oct. 14, 1998.
(60) Provisional application No. 60/105,000, filed on Oct. 20, 1998.
(60) Provisional application No. 60/105,002, filed on Oct. 20, 1998.
(60) Provisional application No. 60/105,266, filed on Oct. 22, 1998.
(60) Provisional application No. 60/105,693, filed on Oct. 26, 1998.
(60) Provisional application No. 60/105,694, filed on Oct. 26, 1998.
(60) Provisional application No. 60/105,807, filed on Oct. 27, 1998.
(60) Provisional application No. 60/105,881, filed on Oct. 27, 1998.
(60) Provisional application No. 60/105,882, filed on Oct. 27, 1998.
(60) Provisional application No. 60/106,023, filed on Oct. 28, 1998.
(60) Provisional application No. 60/106,029, filed on Oct. 28, 1998.
(60) Provisional application No. 60/106,033, filed on Oct. 28, 1998.
(60) Provisional application No. 60/106,062, filed on Oct. 27, 1998.
(60) Provisional application No. 60/106,178, filed on Oct. 28, 1998.
(60) Provisional application No. 60/106,190, filed on Oct. 28, 1998.
(60) Provisional application No. 60/106,248, filed on Oct. 29, 1998.
(60) Provisional application No. 60/106,464, filed on Oct. 30, 1998.
(60) Provisional application No. 60/106,902, filed on Nov. 3, 1998.
(60) Provisional application No. 60/106,905, filed on Nov. 3, 1998.
(60) Provisional application No. 60/106,919, filed on Nov. 3, 1998.
(60) Provisional application No. 60/108,500, filed on Oct. 29, 1998.
(60) Provisional application No. 60/108,775, filed on Nov. 17, 1998.
(60) Provisional application No. 60/108,779, filed on Nov. 17, 1998.
(60) Provisional application No. 60/108,787, filed on Nov. 17, 1998.
(60) Provisional application No. 60/108,788, filed on Nov. 17, 1998.
(60) Provisional application No. 60/108,806, filed on Nov. 17, 1998.
(60) Provisional application No. 60/108,807, filed on Nov. 17, 1998.
(60) Provisional application No. 60/108,848, filed on Nov. 18, 1998.
(60) Provisional application No. 60/108,851, filed on Nov. 18, 1998.
(60) Provisional application No. 60/108,852, filed on Nov. 18, 1998.
(60) Provisional application No. 60/112,419, filed on Dec. 15, 1998.
(60) Provisional application No. 60/112,420, filed on Dec. 15, 1998.
(60) Provisional application No. 60/112,422, filed on Dec. 15, 1998.
(60) Provisional application No. 60/112,514, filed on Dec. 15, 1998.
(60) Provisional application No. 60/112,853, filed on Dec. 16, 1998.
(60) Provisional application No. 60/112,854, filed on Dec. 16, 1998.
(60) Provisional application No. 60/113,011, filed on Dec. 16, 1998.
(60) Provisional application No. 60/113,223, filed on Dec. 22, 1998.
(60) Provisional application No. 60/113,408, filed on Dec. 22, 1998.
(60) Provisional application No. 60/113,621, filed on Dec. 23, 1998.
(60) Provisional application No. 60/114,140, filed on Dec. 23, 1998.
(60) Provisional application No. 60/115,614, filed on Jan. 12, 1999.
(60) Provisional application No. 60/116,527, filed on Jan. 20, 1999.
(60) Provisional application No. 60/119,285, filed on Feb. 9, 1999.
(60) Provisional application No. 60/119,287, filed on Feb. 9, 1999.
(60) Provisional application No. 60/119,525, filed on Feb. 10, 1999.
(60) Provisional application No. 60/119,544, filed on Feb. 10, 1999.
(60) Provisional application No. 60/120,014, filed on Feb. 11, 1999.
(60) Provisional application No. 60/125,774, filed on Mar. 23, 1999.
(60) Provisional application No. 60/125,775, filed on Mar. 23, 1999.
(60) Provisional application No. 60/125,778, filed on Mar. 23, 1999.
(60) Provisional application No. 60/127,035, filed on Mar. 31, 1999.
(60) Provisional application No. 60/127,706, filed on Apr. 5, 1999.
(60) Provisional application No. 60/130,232, filed on Apr. 21, 1999.
(60) Provisional application No. 60/131,022, filed on Apr. 26, 1999.
(60) Provisional application No. 60/131,266, filed on Apr. 27, 1999.
(60) Provisional application No. 60/132,371, filed on May 4, 1999.
(60) Provisional application No. 60/132,381, filed on May 4, 1999.
(60) Provisional application No. 60/132,382, filed on May 4, 1999.
(60) Provisional application No. 60/132,383, filed on May 4, 1999.
(60) Provisional application No. 60/135,741, filed on May 25, 1999.
(60) Provisional application No. 60/135,747, filed on May 25, 1999.
(60) Provisional application No. 60/135,750, filed on May 25, 1999.
(60) Provisional application No. 60/138,387, filed on Jun. 9, 1999.
(60) Provisional application No. 60/140,719, filed on Jun. 22, 1999.
(60) Provisional application No. 60/144,758, filed on Jul. 20, 1999.
(60) Provisional application No. 60/145,070, filed on Jul. 20, 1999.
(60) Provisional application No. 60/146,222, filed on Jul. 28, 1999.
(60) Provisional application No. 60/145,228, filed on Jul. 20, 1999.
(60) Provisional application No. 60/145,698, filed on Jul. 26, 1999.
(60) Provisional application No. 60/146,835, filed on Aug. 3, 1999.
(60) Provisional application No. 60/146,837, filed on Aug. 3, 1999.
(60) Provisional application No. 60/146,845, filed on Aug. 3, 1999.
(60) Provisional application No. 60/146,867, filed on Aug. 3, 1999.
(60) Provisional application No. 60/146,913, filed on Aug. 3, 1999.
(60) Provisional application No. 60/146,918, filed on Aug. 3, 1999.
(60) Provisional application No. 60/146,970, filed on Aug. 3, 1999.
(60) Provisional application No. 60/148,145, filed on Aug. 10, 1999.
(60) Provisional application No. 60/148,158, filed on Aug. 10, 1999.
(60) Provisional application No. 60/148,185, filed on Aug. 10, 1999.
(60) Provisional application No. 60/148,189, filed on Aug. 10, 1999.
(60) Provisional application No. 60/149,268, filed on Aug. 17, 1999.
(60) Provisional application No. 60/149,411, filed on Aug. 17, 1999.
(60) Provisional application No. 60/149,413, filed on Aug. 17, 1999.
(60) Provisional application No. 60/151,736, filed on Aug. 31, 1999.
(60) Provisional application No. 60/151,791, filed on Aug. 31, 1999.
(60) Provisional application No. 60/151,792, filed on Aug. 31, 1999.
(60) Provisional application No. 60/153,805, filed on Sep. 14, 1999.
(60) Provisional application No. 60/153,806, filed on Sep. 14, 1999.
(60) Provisional application No. 60/153,856, filed on Sep. 14, 1999.
(60) Provisional application No. 60/153,857, filed on Sep. 14, 1999.
(60) Provisional application No. 60/153,904, filed on Sep. 14, 1999.
(60) Provisional application No. 60/160,407, filed on Oct. 19, 1999.
(60) Provisional application No. 60/160,408, filed on Oct. 19, 1999.
(60) Provisional application No. 60/162,506, filed on Oct. 29, 1999.
(60) Provisional application No. 60/164,495, filed on Nov. 9, 1999.
(60) Provisional application No. 60/164,499, filed on Nov. 9, 1999.
(60) Provisional application No. 60/164,500, filed on Nov. 9, 1999.
(60) Provisional application No. 60/166,360, filed on Nov. 16, 1999.
(60) Provisional application No. 60/166,363, filed on Nov. 16, 1999.
(60) Provisional application No. 60/166,364, filed on Nov. 16, 1999.
(60) Provisional application No. 60/186,968, filed on Mar. 6, 2000.
(60) Provisional application No. 60/189,320, filed on Mar. 14, 2000.
(60) Provisional application No. 60/189,328, filed on Mar. 14, 2000.
(60) Provisional application No. 60/190,828, filed on Mar. 21, 2000.
(60) Provisional application No. 60/191,007, filed on Mar. 21, 2000.
(60) Provisional application No. 60/191,048, filed on Mar. 21, 2000.
(60) Provisional application No. 60/191,314, filed on Mar. 21, 2000.
(60) Provisional application No. 60/192,655, filed on Mar. 28, 2000.
(60) Provisional application No. 60/193,032, filed on Mar. 29, 2000.
(60) Provisional application No. 60/193,053, filed on Mar. 29, 2000.
(60) Provisional application No. 60/194,449, filed on Apr. 4, 2000.
(60) Provisional application No. 60/194,647, filed on Apr. 4, 2000.
(60) Provisional application No. 60/195,975, filed on Apr. 11, 2000.
(60) Provisional application No. 60/196,000, filed on Apr. 11, 2000.
(60) Provisional application No. 60/196,187, filed on Apr. 11, 2000.
(60) Provisional application No. 60/196,690, filed on Apr. 11, 2000.
(60) Provisional application No. 60/196,820, filed on Apr. 11, 2000.
(60) Provisional application No. 60/198,121, filed on Apr. 18, 2000.
(60) Provisional application No. 60/198,585, filed on Apr. 18, 2000.
(60) Provisional application No. 60/199,397, filed on Apr. 25, 2000.
(60) Provisional application No. 60/199,550, filed on Apr. 25, 2000.
(60) Provisional application No. 60/199,654, filed on Apr. 25, 2000.
(60) Provisional application No. 60/201,516, filed on May 3, 2000.
(60) Provisional application No. 60/204,675, filed on May 17, 2000.

(30) Foreign Application Priority Data
Sep. 16, 1998 (WO) PCT/US98/19330
Oct. 7, 1998 (WO) PCT/US98/21141
Dec. 1, 1998 (WO) PCT/US98/25108
Mar. 8, 1999 (WO) PCT/US99//05028
May 14, 1999 (WO) PCT/US99/10733
Jun. 2, 1999 (WO) PCT/US99/12252
Sep. 1, 1999 (WO) PCT/US99/20111
Sep. 15, 1999 (WO) PCT/US99/21090
Dec. 1, 1999 (WO) PCT/US99/28301
Dec. 2, 1999 (WO) PCT/US99/28551
Dec. 30, 1999 (WO) PCT/US99/31274
Jan. 5, 2000 (WO) PCT/US00/00219
Feb. 18, 2000 (WO) PCT/US00/04341
Feb. 18, 2000 (WO) PCT/US00/04342
Feb. 22, 2000 (WO) PCT/US00/04414
Feb. 24, 2000 (WO) PCT/US00/05004
Mar. 1, 2000 (WO) PCT/US00/05601
Mar. 2, 2000 (WO) PCT/US00/05841
Mar. 15, 2000 (WO) PCT/US00/06884
Mar. 30, 2000 (WO) PCT/US00/08439
May 17, 2000 (WO) PCT/US00/13705
May 22, 2000 (WO) PCT/US00/14042
May 30, 2000 (WO) PCT/US00/14941
Jun. 2, 2000 (WO) PCT/US00/15264
Jul. 28, 2000 (WO) PCT/US00/20710
Aug. 24, 2000 (WO) PCT/US00/23328
Nov. 8, 2000 (WO) PCT/US00/30952
Dec. 1, 2000 (WO) PCT/US00/32678
Dec. 20, 2000 (WO) PCT/US00/34956
Feb. 28, 2001 (WO) PCT/US01/06520
Jun. 1, 2001 (WO) PCT/US01/17800
Jun. 20, 2001 (WO) PCT/US01/19692
Jun. 29, 2001 (WO) PCT/US01/21066
Jul. 9, 2001 (WO) PCT/US01/21735
Aug. 29, 2001 (WO) PCT/US01/27099

(51) Int. Cl. C12P 21/02; C12N 5/06; C07K 14/47; C07H 21/04; C12N 9/00

(52) U.S. Cl. 435/69.1; 435/320.1; 435/183; 435/325; 530/350; 536/23.2

(57) ABSTRACT

The present invention is directed to novel polypeptides and to nucleic acid molecules encoding those polypeptides. Also provided herein are vectors and host cells comprising those nucleic acid sequences, chimeric polypeptide molecules comprising the polypeptides of the present invention fused to heterologous polypeptide sequences, antibodies which bind to the polypeptides of the present invention and to methods for producing the polypeptides of the present invention.

### FIELD OF THE INVENTION

The present invention relates generally to the identification and isolation of novel DNA and to the recombinant production of novel polypeptides.

### BACKGROUND OF THE INVENTION

Extracellular proteins play important roles in, among other things, the formation, differentiation and maintenance of multicellular organisms. The fate of many individual cells, e.g., proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, received and interpreted by diverse cell receptors or membrane-bound proteins. These secreted polypeptides or signaling molecules normally pass through the cellular secretory pathway to reach their site of action in the extracellular environment.

Secreted proteins have various industrial applications, including as pharmaceuticals, diagnostics, biosensors and bioreactors. Most protein drugs available at present, such as thrombolytic agents, interferons, interleukins, erythropoietins, colony stimulating factors, and various other cytokines, are secretory proteins. Their receptors, which are membrane proteins, also have potential as therapeutic or diagnostic agents. Efforts are being undertaken by both industry and academia to identify new, native secreted proteins. Many efforts are focused on the screening of mammalian recombinant DNA libraries to identify the coding sequences for novel secreted proteins. Examples of screening methods and techniques are described in the literature [see, for example, Klein et al., Proc. Natl. Acad. Sci. 93:7108-7113 (1996); U.S. Pat. No. 5,536,637)].

Membrane-bound proteins and receptors can play important roles in, among other things, the formation, differentiation and maintenance of multicellular organisms. The fate of many individual cells, e.g., proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, received and interpreted by diverse cell receptors or membrane-bound proteins. Such membrane-bound proteins and cell receptors include, but are not limited to, cytokine receptors, receptor kinases, receptor phosphatases, receptors involved in cell-cell interactions, and cellular adhesin molecules like selectins and integrins. For instance, transduction of signals that regulate cell growth and differentiation is regulated in part by phosphorylation of various cellular proteins. Protein tyrosine kinases, enzymes that catalyze that process, can also act as growth factor receptors. Examples include fibroblast growth factor receptor and nerve growth factor receptor.

Membrane-bound proteins and receptor molecules have various industrial applications, including as pharmaceutical and diagnostic agents. Receptor immunoadhesins, for instance, can be employed as therapeutic agents to block receptor-ligand interactions. The membrane-bound proteins can also be employed for screening of potential peptide or small molecule inhibitors of the relevant receptor/ligand interaction.

Efforts are being undertaken by both industry and academia to identify new, native receptor or membrane-bound proteins. Many efforts are focused on the screening of mammalian recombinant DNA libraries to identify the coding sequences for novel receptor or membrane-bound proteins.

### SUMMARY OF THE INVENTION

In one embodiment, the invention provides an isolated nucleic acid molecule comprising a nucleotide sequence that encodes a PRO polypeptide.

In one aspect, the isolated nucleic acid molecule comprises a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule encoding a PRO polypeptide having a full-length amino acid sequence as disclosed herein, an amino acid sequence lacking the signal peptide as disclosed herein, an extracellular domain of a transmembrane protein, with or without the signal peptide, as disclosed herein or any other specifically defined fragment of the full-length amino acid sequence as disclosed herein, or (b) the complement of the DNA molecule of (a).

In other aspects, the isolated nucleic acid molecule comprises a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule comprising the coding sequence of a full-length PRO polypeptide cDNA as disclosed herein, the coding sequence of a PRO polypeptide lacking the signal peptide as disclosed herein, the coding sequence of an extracellular domain of a transmembrane PRO polypeptide, with or without the signal peptide, as disclosed herein or the coding sequence of any other specifically defined fragment of the full-length amino acid sequence as disclosed herein, or (b) the complement of the DNA molecule of (a).

In a further aspect, the invention concerns an isolated nucleic acid molecule comprising a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule that encodes the same mature polypeptide encoded by any of the human protein cDNAs deposited with the ATCC as disclosed herein, or (b) the complement of the DNA molecule of (a).

Another aspect the invention provides an isolated nucleic acid molecule comprising a nucleotide sequence encoding a PRO polypeptide which is either transmembrane domain-deleted or transmembrane domain-inactivated, or is complementary to such encoding nucleotide sequence, wherein the transmembrane domain(s) of such polypeptide are disclosed herein. Therefore, soluble extracellular domains of the herein described PRO polypeptides are contemplated.

Another embodiment is directed to fragments of a PRO polypeptide coding sequence, or the complement thereof, that may find use as, for example, hybridization probes, for encoding fragments of a PRO polypeptide that may optionally encode a polypeptide comprising a binding site for an anti-PRO antibody or as antisense oligonucleotide probes. Such nucleic acid fragments are usually at least about 10 nucleotides in length, alternatively at least about 15 nucleotides in length, alternatively at least about 20 nucleotides in length, alternatively at least about 30 nucleotides in length, alternatively at least about 40 nucleotides in length, alternatively at least about 50 nucleotides in length, alternatively at least about 60 nucleotides in length, alternatively at least about 70 nucleotides in length, alternatively at least about 80 nucleotides in length, alternatively at least about 90 nucleotides in length, alternatively at least about 100 nucleotides in length, alternatively at least about 110 nucleotides in length, alternatively at least about 120 nucleotides in length, alternatively at least about 130 nucleotides in length, alternatively at least about 140 nucleotides in length, alternatively at least about 150 nucleotides in length, alternatively at least about 160 nucleotides in length, alternatively at least about 170 nucleotides in length, alternatively at least about 180 nucleotides in length, alternatively at least about 190 nucleotides in length, alternatively at least about 200 nucleotides in length, alternatively at least about 250 nucleotides in length, alternatively at least about 300 nucleotides in length, alternatively at least about 350 nucleotides in length, alternatively at least about 400 nucleotides in length, alternatively at least about 450 nucleotides in length, alternatively at least about 500 nucleotides in length, alternatively at least about 600 nucleotides in length, alternatively at least about 700 nucleotides in length, alternatively at least about 800 nucleotides in length, alternatively at least about 900 nucleotides in length and alternatively at least about 1000 nucleotides in length, wherein in this context the term “about” means the referenced nucleotide sequence length plus or minus 10% of that referenced length. It is noted that novel fragments of a PRO polypeptide-encoding nucleotide sequence may be determined in a routine manner by aligning the PRO polypeptide-encoding nucleotide sequence with other known nucleotide sequences using any of a number of well known sequence alignment programs and determining which PRO polypeptide-encoding nucleotide sequence fragment(s) are novel. All of such PRO polypeptide-encoding nucleotide sequences are contemplated herein. Also contemplated are the PRO polypeptide fragments encoded by these nucleotide molecule fragments, preferably those PRO polypeptide fragments that comprise a binding site for an anti-PRO antibody.

In another embodiment, the invention provides isolated PRO polypeptide encoded by any of the isolated nucleic acid sequences hereinabove identified.

In a certain aspect, the invention concerns an isolated PRO polypeptide, comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a PRO polypeptide having a full-length amino acid sequence as disclosed herein, an amino acid sequence lacking the signal peptide as disclosed herein, an extracellular domain of a transmembrane protein, with or without the signal peptide, as disclosed herein or any other specifically defined fragment of the full-length amino acid sequence as disclosed herein.

In a further aspect, the invention concerns an isolated PRO polypeptide comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to an amino acid sequence encoded by any of the human protein cDNAs deposited with the ATCC as disclosed herein.

In a specific aspect, the invention provides an isolated PRO polypeptide without the N-terminal signal sequence and/or the initiating methionine and is encoded by a nucleotide sequence that encodes such an amino acid sequence as hereinbefore described. Processes for producing the same are also herein described, wherein those processes comprise culturing a host cell comprising a vector which comprises the appropriate encoding nucleic acid molecule under conditions suitable for expression of the PRO polypeptide and recovering the PRO polypeptide from the cell culture.

Another aspect the invention provides an isolated PRO polypeptide which is either transmembrane domain-deleted or transmembrane domain-inactivated. Processes for producing the same are also herein described, wherein those processes comprise culturing a host cell comprising a vector which comprises the appropriate encoding nucleic acid molecule under conditions suitable for expression of the PRO polypeptide and recovering the PRO polypeptide from the cell culture.

In yet another embodiment, the invention concerns agonists and antagonists of a native PRO polypeptide as defined herein. In a particular embodiment, the agonist or antagonist is an anti-PRO antibody or a small molecule.

In a further embodiment, the invention concerns a method of identifying agonists or antagonists to a PRO polypeptide which comprise contacting the PRO polypeptide with a candidate molecule and monitoring a biological activity mediated by said PRO polypeptide. Preferably, the PRO polypeptide is a native PRO polypeptide.

In a still further embodiment, the invention concerns a composition of matter comprising a PRO polypeptide, or an agonist or antagonist of a PRO polypeptide as herein described, or an anti-PRO antibody, in combination with a carrier. Optionally, the carrier is a pharmaceutically acceptable carrier.

Another embodiment of the present invention is directed to the use of a PRO polypeptide, or an agonist or antagonist thereof as hereinbefore described, or an anti-PRO antibody, for the preparation of a medicament useful in the treatment of a condition which is responsive to the PRO polypeptide, an agonist or antagonist thereof or an anti-PRO antibody.

In other embodiments of the present invention, the invention provides vectors comprising DNA encoding any of the herein described polypeptides. Host cell comprising any such vector are also provided. By way of example, the host cells may be CHO cells, E. coli, or yeast. A process for producing any of the herein described polypeptides is further provided and comprises culturing host cells under conditions suitable for expression of the desired polypeptide and recovering the desired polypeptide from the cell culture.

In other embodiments, the invention provides chimeric molecules comprising any of the herein described polypeptides fused to a heterologous polypeptide or amino acid sequence. Example of such chimeric molecules comprise any of the herein described polypeptides fused to an epitope tag sequence or a Fc region of an immunoglobulin.

In another embodiment, the invention provides an antibody which binds, preferably specifically, to any of the above or below described polypeptides. Optionally, the antibody is a monoclonal antibody, humanized antibody, antibody fragment or single-chain antibody.

In yet other embodiments, the invention provides oligonucleotide probes which may be useful for isolating genomic and cDNA nucleotide sequences, measuring or detecting expression of an associated gene or as antisense probes, wherein those probes may be derived from any of the above or below described nucleotide sequences. Preferred probe lengths are described above.

In yet other embodiments, the present invention is directed to methods of using the PRO polypeptides of the present invention for a variety of uses based upon the functional biological assay data presented in the Examples below.

### BRIEF DESCRIPTION OF THE DRAWINGS

FIG. 1 shows a nucleotide sequence (SEQ ID NO: 1) of a native sequence PRO276 cDNA, wherein SEQ ID NO: 1 is a clone designated herein as “DNA16435-1208”.

FIG. 2 shows the amino acid sequence (SEQ ID NO: 2) derived from the coding sequence of SEQ ID NO: 1 shown in FIG. 1.

FIG. 3 shows a nucleotide sequence (SEQ ID NO: 3) of a native sequence PRO284 cDNA, wherein SEQ ID NO: 3 is a clone designated herein as “DNA23318-1211”.

FIG. 4 shows the amino acid sequence (SEQ ID NO: 4) derived from the coding sequence of SEQ ID NO: 3 shown in FIG. 3.

FIG. 5 shows a nucleotide sequence (SEQ ID NO: 5) of a native sequence PRO193 cDNA, wherein SEQ ID NO: 5 is a clone designated herein as “DNA23322-1393”.

FIG. 6 shows the amino acid sequence (SEQ ID NO: 6) derived from the coding sequence of SEQ ID NO: 5 shown in FIG. 5.

FIG. 7 shows a nucleotide sequence (SEQ ID NO: 7) of a native sequence PRO190 cDNA, wherein SEQ ID NO: 7 is a clone designated herein as “DNA23334-1392”.

FIG. 8 shows the amino acid sequence (SEQ ID NO: 8) derived from the coding sequence of SEQ ID NO: 7 shown in FIG. 7.

FIG. 9 shows a nucleotide sequence (SEQ ID NO: 9) of a native sequence PRO180 cDNA, wherein SEQ ID NO: 9 is a clone designated herein as “DNA26843-1389”.

FIG. 10 shows the amino acid sequence (SEQ ID NO: 10) derived from the coding sequence of SEQ ID NO: 9 shown in FIG. 9.

FIG. 11 shows a nucleotide sequence (SEQ ID NO: 11) of a native sequence PRO194 cDNA, wherein SEQ ID NO: 11 is a clone designated herein as “DNA26844-1394”.

FIG. 12 shows the amino acid sequence (SEQ ID NO: 12) derived from the coding sequence of SEQ ID NO: 11 shown in FIG. 11.

FIG. 13 shows a nucleotide sequence (SEQ ID NO: 13) of a native sequence PRO218 cDNA, wherein SEQ ID NO: 13 is a clone designated herein as “DNA30867-1335”.

FIG. 14 shows the amino acid sequence (SEQ ID NO: 14) derived from the coding sequence of SEQ ID NO: 13 shown in FIG. 13.

FIG. 15 shows a nucleotide sequence (SEQ ID NO: 15) of a native sequence PRO260 cDNA, wherein SEQ ID NO: 15 is a clone designated herein as “DNA33470-1175”.

FIG. 16 shows the amino acid sequence (SEQ ID NO: 16) derived from the coding sequence of SEQ ID NO: 15 shown in FIG. 15.

FIG. 17 shows a nucleotide sequence (SEQ ID NO: 17) of a native sequence PRO233 cDNA, wherein SEQ ID NO: 17 is a clone designated herein as “DNA34436-1238”.

FIG. 18 shows the amino acid sequence (SEQ ID NO: 18) derived from the coding sequence of SEQ ID NO: 17 shown in FIG. 17.

FIG. 19 shows a nucleotide sequence (SEQ ID NO: 19) of a native sequence PRO234 cDNA, wherein SEQ ID NO: 19 is a clone designated herein as “DNA35557-1137”.

FIG. 20 shows the amino acid sequence (SEQ ID NO: 20) derived from the coding sequence of SEQ ID NO: 19 shown in FIG. 19.

FIG. 21 shows a nucleotide sequence (SEQ ID NO: 21) of a native sequence PRO236 cDNA, wherein SEQ ID NO: 21 is a clone designated herein as “DNA35599-1168”.

FIG. 22 shows the amino acid sequence (SEQ ID NO: 22) derived from the coding sequence of SEQ ID NO: 21 shown in FIG. 21.

FIG. 23 shows a nucleotide sequence (SEQ ID NO: 23) of a native sequence PRO244 cDNA, wherein SEQ ID NO: 23 is a clone designated herein as “DNA35668-1171”.

FIG. 24 shows the amino acid sequence (SEQ ID NO: 24) derived from the coding sequence of SEQ ID NO: 23 shown in FIG. 23.

FIG. 25 shows a nucleotide sequence (SEQ ID NO: 25) of a native sequence PRO262 cDNA, wherein SEQ ID NO: 25 is a clone designated herein as “DNA36992-1168”.

FIG. 26 shows the amino acid sequence (SEQ ID NO: 26) derived from the coding sequence of SEQ ID NO: 25 shown in FIG. 25.

FIG. 27 shows a nucleotide sequence (SEQ ID NO: 27) of a native sequence PRO271 cDNA, wherein SEQ ID NO: 27 is a clone designated herein as “DNA39423-1182”.

FIG. 28 shows the amino acid sequence (SEQ ID NO: 28) derived from the coding sequence of SEQ ID NO: 27 shown in FIG. 27.

FIG. 29 shows a nucleotide sequence (SEQ ID NO: 29) of a native sequence PRO268 cDNA, wherein SEQ ID NO: 29 is a clone designated herein as “DNA39427-1179”.

FIG. 30 shows the amino acid sequence (SEQ ID NO: 30) derived from the coding sequence of SEQ ID NO: 29 shown in FIG. 29.

FIG. 31 shows a nucleotide sequence (SEQ ID NO: 31) of a native sequence PRO270 cDNA, whereinSEQ ID NO: 31 is a clone designated herein as “DNA39510-1181”.

FIG. 32 shows the amino acid sequence (SEQ ID NO: 32) derived from the coding sequence of SEQ ID NO: 31 shown in FIG. 31.

FIG. 33 shows a nucleotide sequence (SEQ ID NO: 33) of a native sequence PRO355 cDNA, wherein SEQ ID NO: 33 is a clone designated herein as “DNA39518-1247”.

FIG. 34 shows the amino acid sequence (SEQ ID NO: 34) derived from the coding sequence of SEQ ID NO: 33 shown in FIG. 33.

FIG. 35 shows a nucleotide sequence (SEQ ID NO: 35) of a native sequence PRO298 cDNA, wherein SEQ ID NO: 35 is a clone designated herein as “DNA39975-1210”.

FIG. 36 shows the amino acid sequence (SEQ ID NO: 36) derived from the coding sequence of SEQ ID NO: 35 shown in FIG. 35.

FIG. 37 shows a nucleotide sequence (SEQ ID NO: 37) of a native sequence PRO299 cDNA, wherein SEQ ID NO: 37 is a clone designated herein as “DNA39976-1215”.

FIG. 38 shows the amino acid sequence (SEQ ID NO: 38) derived from the coding sequence of SEQ ID NO: 37 shown in FIG. 37.

FIG. 39 shows a nucleotide sequence (SEQ ID NO: 39) of a native sequence PRO296 cDNA, wherein SEQ ID NO: 39 is a clone designated herein as “DNA39979-1213”.

FIG. 40 shows the amino acid sequence (SEQ ID NO: 40) derived from the coding sequence of SEQ ID NO: 39 shown in FIG. 39.

FIG. 41 shows a nucleotide sequence (SEQ ID NO: 4 1) of a native sequence PRO329 cDNA, wherein SEQ ID NO: 41 is a clone designated herein as “DNA40594-1233”.

FIG. 42 shows the amino acid sequence (SEQ ID NO: 42) derived from the coding sequence of SEQ ID NO: 41 shown in FIG. 41.

FIG. 43 shows a nucleotide sequence (SEQ ID NO: 43) of a native sequence PRO330 cDNA, wherein SEQ ID NO: 43 is a clone designated herein as “DNA40603-1232”.

FIG. 44 shows the amino acid sequence (SEQ ID NO: 44) derived from the coding sequence of SEQ ID NO: 43 shown in FIG. 43.

FIG. 45 shows a nucleotide sequence (SEQ ID NO: 45) of a native sequence PRO294 cDNA, wherein SEQ ID NO: 45 is a clone designated herein as “DNA40604-1187”.

FIG. 46 shows the amino acid sequence (SEQ ID NO: 46) derived from the coding sequence of SEQ ID NO: 45 shown in FIG. 45.

FIG. 47 shows a nucleotide sequence (SEQ ID NO: 47) of a native sequence PRO300 cDNA, wherein SEQ ID NO: 47 is a clone designated herein as “DNA40625-1189”.

FIG. 48 shows the amino acid sequence (SEQ ID NO: 48) derived from the coding sequence of SEQ ID NO: 47 shown in FIG. 47.

FIG. 49 shows a nucleotide sequence (SEQ ID NO: 49) of a native sequence PRO307 cDNA, wherein SEQ ID NO: 49 is a clone designated herein as “DNA41225-1217”.

FIG. 50 shows the amino acid sequence (SEQ ID NO: 50) derived from the coding sequence of SEQ ID NO: 49 shown in FIG. 49.

FIG. 51 shows a nucleotide sequence (SEQ ID NO: 51) of a native sequence PRO334 cDNA, wherein SEQ ID NO: 51 is a clone designated herein as “DNA41379-1236”.

FIG. 52 shows the amino acid sequence (SEQ ID NO: 52) derived from the coding sequence of SEQ ID NO: 51 shown in FIG. 51.

FIG. 53 shows a nucleotide sequence (SEQ ID NO: 53) of a native sequence PRO352 cDNA, wherein SEQ ID NO: 53 is a clone designated herein as “DNA41386-1316”.

FIG. 54 shows the amino acid sequence (SEQ ID NO: 54) derived from the coding sequence of SEQ ID NO: 53 shown in FIG. 53.

FIG. 55 shows a nucleotide sequence (SEQ ID NO: 55) of a native sequence PRO710 cDNA, wherein SEQ ID NO: 55 is a clone designated herein as “DNA44161-1434”.

FIG. 56 shows the amino acid sequence (SEQ ID NO: 56) derived from the coding sequence of SEQ ID NO: 55 shown in FIG. 55.

FIG. 57 shows a nucleotide sequence (SEQ ID NO: 57) of a native sequence PRO873 cDNA, wherein SEQ ID NO: 57 is a clone designated herein as “DNA44179-1362”.

FIG. 58 shows the amino acid sequence (SEQ ID NO: 58) derived from the coding sequence of SEQ ID NO: 57 shown in FIG. 57.

FIG. 59 shows a nucleotide sequence (SEQ ID NO: 59) of a native sequence PRO354 cDNA, wherein SEQ ID NO: 59 is a clone designated herein as “DNA44192-1246”.

FIG. 60 shows the amino acid sequence (SEQ ID NO: 60) derived from the coding sequence of SEQ ID NO: 59 shown in FIG. 59.

FIG. 61 shows a nucleotide sequence (SEQ ID NO: 61) of a native sequence PRO1151 cDNA, wherein SEQ ID NO: 61 is a clone designated herein as “DNA44694-1500”.

FIG. 62 shows the amino acid sequence (SEQ ID NO: 62) derived from the coding sequence of SEQ ID NO: 61 shown in FIG. 61.

FIG. 63 shows a nucleotide sequence (SEQ ID NO: 63) of a native sequence PRO382 cDNA, wherein SEQ ID NO: 63 is a clone designated herein as “DNA45234-1277”.

FIG. 64 shows the amino acid sequence (SEQ ID NO: 64) derived from the coding sequence of SEQ ID NO: 63 shown in FIG. 63.

FIG. 65 shows a nucleotide sequence (SEQ ID NO: 65) of a native sequence PRO1864 cDNA, wherein SEQ ID NO: 65 is a clone designated herein as “DNA45409-2511”.

FIG. 66 shows the amino acid sequence (SEQ ID NO: 66) derived from the coding sequence of SEQ ID NO: 65 shown in FIG. 65.

FIG. 67 shows a nucleotide sequence (SEQ ID NO: 67) of a native sequence PRO386 cDNA, wherein SEQ ID NO: 67 is a clone designated herein as “DNA45415-1318”.

FIG. 68 shows the amino acid sequence (SEQ ID NO: 68) derived from the coding sequence of SEQ ID NO: 67 shown in FIG. 67.

FIG. 69 shows a nucleotide sequence (SEQ ID NO: 69) of a native sequence PRO541 cDNA, wherein SEQ ID NO: 69 is a clone designated herein as “DNA45417-1432”.

FIG. 70 shows the amino acid sequence (SEQ ID NO: 70) derived from the coding sequence of SEQ ID NO: 69 shown in FIG. 69.

FIG. 71 shows a nucleotide sequence (SEQ ID NO: 71) of a native sequence PRO852 cDNA, wherein SEQ ID NO: 71 is a clone designated herein as “DNA45493-1349”.

FIG. 72 shows the amino acid sequence (SEQ ID NO: 72) derived from the coding sequence of SEQ ID NO: 71 shown in FIG. 71.

FIG. 73 shows a nucleotide sequence (SEQ ID NO: 73) of a native sequence PRO700 cDNA, wherein SEQ ID NO: 73 is a clone designated herein as “DNA46776-1284”.

FIG. 74 shows the amino acid sequence (SEQ ID NO: 74) derived from the coding sequence of SEQ ID NO: 73 shown in FIG. 73.

FIGS. 75A-75B show a nucleotide sequence (SEQ ID NO: 75) of a native sequence PRO708 cDNA, wherein SEQ ID NO: 75 is a clone designated herein as “DNA48296-1292”.

FIG. 76 shows the amino acid sequence (SEQ ID NO: 76) derived from the coding sequence of SEQ ID NO: 75 shown in FIGS. 75A-75B.

FIG. 77 shows a nucleotide sequence (SEQ ID NO: 77) of a native sequence PRO707 cDNA, wherein SEQ ID NO: 77 is a clone designated herein as “DNA48306-1291”.

FIG. 78 shows the amino acid sequence (SEQ ID NO: 78) derived from the coding sequence of SEQ ID NO: 77 shown in FIG. 77.

FIG. 79 shows a nucleotide sequence (SEQ ID NO: 79) of a native sequence PRO864 cDNA, wherein SEQ ID NO: 79 is a clone designated herein as “DNA48328-1355”.

FIG. 80 shows the amino acid sequence (SEQ ID NO: 80) derived from the coding sequence of SEQ ID NO: 79 shown in FIG. 79.

FIG. 81 shows a nucleotide sequence (SEQ ID NO: 81) of a native sequence PRO706 cDNA, wherein SEQ ID NO: 81 is a clone designated herein as “DNA48329-1290”.

FIG. 82 shows the amino acid sequence (SEQ ID NO: 82) derived from the coding sequence of SEQ ID NO: 81 shown in FIG. 81.

FIG. 83 shows a nucleotide sequence (SEQ ID NO: 83) of a native sequence PRO732 cDNA, wherein SEQ ID NO: 83 is a clone designated herein as “DNA48334-1435”.

FIG. 84 shows the amino acid sequence (SEQ ID NO: 84) derived from the coding sequence of SEQ ID NO: 83 shown in FIG. 83.

FIG. 85 shows a nucleotide sequence (SEQ ID NO: 85) of a native sequence PRO537 cDNA, wherein SEQ ID NO: 85 is a clone designated herein as “DNA49141-1431”.

FIG. 86 shows the amino acid sequence (SEQ ID NO: 86) derived from the coding sequence of SEQ ID NO: 85 shown in FIG. 85.

FIG. 87 shows a nucleotide sequence (SEQ ID NO: 87) of a native sequence PRO545 cDNA, wherein SEQ ID NO: 87 is a clone designated herein as “DNA49624-1279”.

FIG. 88 shows the amino acid sequence (SEQ ID NO: 88) derived from the coding sequence of SEQ ID NO: 87 shown in FIG. 87.

FIG. 89 shows a nucleotide sequence (SEQ ID NO: 89) of a native sequence PRO718 cDNA, wherein SEQ ID NO: 89 is a clone designated herein as “DNA49647-1398”.

FIG. 90 shows the amino acid sequence (SEQ ID NO: 90) derived from the coding sequence of SEQ ID NO: 89 shown in FIG. 89.

FIG. 91 shows a nucleotide sequence (SEQ ID NO: 91) of a native sequence PRO872 cDNA, wherein SEQ ID NO: 91 is a clone designated herein as “DNA49819-1439”.

FIG. 92 shows the amino acid sequence (SEQ ID NO: 92) derived from the coding sequence of SEQ ID NO: 91 shown in FIG. 91.

FIG. 93 shows a nucleotide sequence (SEQ ID NO: 93) of a native sequence PRO704 cDNA, wherein SEQ ID NO: 93 is a clone designated herein as “DNA50911-1288”.

FIG. 94 shows the amino acid sequence (SEQ ID NO: 94) derived from the coding sequence of SEQ ID NO: 93 shown in FIG. 93.

FIG. 95 shows a nucleotide sequence (SEQ ID NO: 95) of a native sequence PRO705 cDNA, wherein SEQ ID NO: 95 is a clone designated herein as “DNA50914-1289”.

FIG. 96 shows the amino acid sequence (SEQ ID NO: 96) derived from the coding sequence of SEQ ID NO: 95 shown in FIG. 95.

FIG. 97 shows a nucleotide sequence (SEQ ID NO: 97) of a native sequence PRO871 cDNA, wherein SEQ ID NO: 97 is a clone designated herein as “DNA50919-1361”.

FIG. 98 shows the amino acid sequence (SEQ ID NO: 98) derived from the coding sequence of SEQ ID NO: 97 shown in FIG. 97.

FIG. 99 shows a nucleotide sequence (SEQ ID NO: 99) of a native sequence PRO702 cDNA, wherein SEQ ID NO: 99 is a clone designated herein as “DNA50980-1286”.

FIG. 100 shows the amino acid sequence (SEQ ID NO: 100) derived from the coding sequence of SEQ ID NO: 99 shown in FIG. 99.

FIG. 101 shows a nucleotide sequence (SEQ ID NO: 101) of a native sequence PRO944 cDNA, wherein SEQ ID NO: 101 is a clone designated herein as “DNA52185-1370”.

FIG. 102 shows the amino acid sequence (SEQ ID NO: 102) derived from the coding sequence of SEQ ID NO: 101 shown in FIG. 101.

FIG. 103 shows a nucleotide sequence (SEQ ID NO: 103) of a native sequence PRO739 cDNA, wherein SEQ ID NO: 103 is a clone designated herein as “DNA52756”.

FIG. 104 shows the amino acid sequence (SEQ ID NO: 104) derived from the coding sequence of SEQ ID NO: 103 shown in FIG. 103.

FIG. 105 shows a nucleotide sequence (SEQ ID NO: 105) of a native sequence PRO941 cDNA, wherein SEQ ID NO: 105 is a clone designated herein as “DNA53906-1368”.

FIG. 106 shows the amino acid sequence (SEQ ID NO: 106) derived from the coding sequence of SEQ ID NO: 105 shown in FIG. 105.

FIG. 107 shows a nucleotide sequence (SEQ ID NO: 107) of a native sequence PRO1082 cDNA, wherein SEQ ID NO: 107 is a clone designated herein as “DNA53912-1457”.

FIG. 108 shows the amino acid sequence (SEQ ID NO: 108) derived from the coding sequence of SEQ ID NO: 107 shown in FIG. 107.

FIG. 109 shows a nucleotide sequence (SEQ ID NO: 109) of a native sequence PRO1133 cDNA, wherein SEQ ID NO: 109 is a clone designated herein as “DNA53913-1490”.

FIG. 110 shows the amino acid sequence (SEQ ID NO: 110) derived from the coding sequence of SEQ ID NO: 109 shown in FIG. 109.

FIG. 111 shows a nucleotide sequence (SEQ ID NO: 111) of a native sequence PRO983 cDNA, wherein SEQ ID NO: 111 is a clone designated herein as “DNA53977-1371”.

FIG. 112 shows the amino acid sequence (SEQ ID NO: 112) derived from the coding sequence of SEQ ID NO: 111 shown in FIG. 111.

FIG. 113 shows a nucleotide sequence (SEQ ID NO: 113) of a native sequence PRO784 cDNA, wherein SEQ ID NO: 1 13 is a clone designated herein as “DNA53978-1443”.

FIG. 114 shows the amino acid sequence (SEQ ID NO: 114) derived from the coding sequence of SEQ ID NO: 113 shown in FIG. 113.

FIG. 115 shows a nucleotide sequence (SEQ ID NO: 115) of a native sequence PRO783 cDNA, wherein SEQ ID NO: 115 is a clone designated herein as “DNA53996-1442”.

FIG. 116 shows the amino acid sequence (SEQ ID NO: 116) derived from the coding sequence of SEQ ID NO: 115 shown in FIG. 115.

FIG. 117 shows a nucleotide sequence (SEQ ID NO: 117) of a native sequence PRO940 cDNA, wherein SEQ ID NO: 117 is a clone designated herein as “DNA54002-1367”.

FIG. 118 shows the amino acid sequence (SEQ ID NO: 118) derived from the coding sequence of SEQ ID NO: 117 shown in FIG. 117.

FIG. 119 shows a nucleotide sequence (SEQ ID NO: 119) of a native sequence PRO768 cDNA, wherein SEQ ID NO: 119 is a clone designated herein as “DNA55737-1345”.

FIG. 120 shows the amino acid sequence (SEQ ID NO: 120) derived from the coding sequence of SEQ ID NO: 119 shown in FIG. 119.

FIG. 121 shows a nucleotide sequence (SEQ ID NO: 121) of a native sequence PRO1079 cDNA, wherein SEQ ID NO: 121 is a clone designated herein as “DNA56050-1455”.

FIG. 122 shows the amino acid sequence (SEQ ID NO: 122) derived from the coding sequence of SEQ ID NO: 121 shown in FIG. 121.

FIG. 123 shows a nucleotide sequence (SEQ ID NO: 123) of a native sequence PRO1078 cDNA, wherein SEQ ID NO: 123 is a clone designated herein as “DNA56052-1454”.

FIG. 124 shows the amino acid sequence (SEQ ID NO: 124) derived from the coding sequence of SEQ ID NO: 123 shown in FIG. 123.

FIG. 125 shows a nucleotide sequence (SEQ ID NO: 125) of a native sequence PRO1018 cDNA, wherein SEQ ID NO: 125 is a clone designated herein as “DNA56107-1415”.

FIG. 126 shows the amino acid sequence (SEQ ID NO: 126) derived from the coding sequence of SEQ ID NO: 125 shown in FIG. 125.

FIG. 127 shows a nucleotide sequence (SEQ ID NO: 127) of a native sequence PRO793 cDNA, wherein SEQ ID NO: 127 is a clone designated herein as “DNA56110-1437”.

FIG. 128 shows the amino acid sequence (SEQ ID NO: 128) derived from the coding sequence of SEQ ID NO: 127 shown in FIG. 127.

FIG. 129 shows a nucleotide sequence (SEQ ID NO: 129) of a native sequence PRO 1773 cDNA, wherein SEQ ID NO: 129 is a clone designated herein as “DNA56406-1704”.

FIG. 130 shows the amino acid sequence (SEQ ID NO: 130) derived from the coding sequence of SEQ ID NO: 129 shown in FIG. 129.

FIG. 131 shows a nucleotide sequence (SEQ ID NO: 131) of a native sequence PRO1014 cDNA, wherein SEQ ID NO: 131 is a clone designated herein as “DNA56409-1377”.

FIG. 132 shows the amino acid sequence (SEQ ID NO: 132) derived from the coding sequence of SEQ ID NO: 131 shown in FIG. 131.

FIG. 133 shows a nucleotide sequence (SEQ ID NO: 133) of a native sequence PRO1013 cDNA, wherein SEQ ID NO: 133 is a clone designated herein as “DNA56410-1414”.

FIG. 134 shows the amino acid sequence (SEQ ID NO: 134) derived from the coding sequence of SEQ ID NO: 133 shown in FIG. 133.

FIG. 135 shows a nucleotide sequence (SEQ ID NO: 135) of a native sequence PRO937 cDNA, wherein SEQ ID NO: 135 is a clone designated herein as “DNA56436-1448”.

FIG. 136 shows the amino acid sequence (SEQ ID NO: 136) derived from the coding sequence of SEQ ID NO: 135 shown in FIG. 135.

FIG. 137 shows a nucleotide sequence (SEQ ID NO: 137) of a native sequence PRO 1477 cDNA, wherein SEQ ID NO: 137 is a clone designated herein as “DNA56529-1647”.

FIG. 138 shows the amino acid sequence (SEQ ID NO: 138) derived from the coding sequence of SEQ ID NO: 137 shown in FIG. 137.

FIG. 139 shows a nucleotide sequence (SEQ ID NO: 139) of a native sequence PRO842 cDNA, wherein SEQ ID NO: 139 is a clone designated herein as “DNA56855-1447”.

FIG. 140 shows the amino acid sequence (SEQ ID NO: 140) derived from the coding sequence of SEQ ID NO: 139 shown in FIG. 139.

FIG. 141 shows a nucleotide sequence (SEQ ID NO: 141) of a native sequence PRO839 cDNA, wherein SEQ ID NO: 141 is a clone designated herein as “DNA56859-1445”.

FIG. 142 shows the amino acid sequence (SEQ ID NO: 142) derived from the coding sequence of SEQ ID NO: 141 shown in FIG. 141.

FIG. 143 shows a nucleotide sequence (SEQ ID NO: 143) of a native sequence PRO1180 cDNA, wherein SEQ ID NO: 143 is a clone designated herein as “DNA56860-1510”.

FIG. 144 shows the amino acid sequence (SEQ ID NO: 144) derived from the coding sequence of SEQ ID NO: 143 shown in FIG. 143.

FIG. 145 shows a nucleotide sequence (SEQ ID NO: 145) of a native sequence PRO1134 cDNA, wherein SEQ ID NO: 145 is a clone designated herein as “DNA56865-1491”.

FIG. 146 shows the amino acid sequence (SEQ ID NO: 146) derived from the coding sequence of SEQ ID NO: 145 shown in FIG. 145.

FIG. 147 shows a nucleotide sequence (SEQ ID NO: 147) of a native sequence PRO 1115 cDNA, wherein SEQ ID NO: 147 is a clone designated herein as “DNA56868-1478”.

FIG. 148 shows the amino acid sequence (SEQ ID NO: 148) derived from the coding sequence of SEQ ID NO: 147 shown in FIG. 147.

FIG. 149 shows a nucleotide sequence (SEQ ID NO: 149) of a native sequence PRO1277 cDNA, wherein SEQ ID NO: 149 is a clone designated herein as “DNA56869-1545”.

FIG. 150 shows the amino acid sequence (SEQ ID NO: 150) derived from the coding sequence of SEQ ID NO: 149 shown in FIG. 149.

FIG. 151 shows a nucleotide sequence (SEQ ID NO: 151) of a native sequence PRO1135 cDNA, wherein SEQ ID NO: 151 is a clone designated herein as “DNA56870-1492”.

FIG. 152 shows the amino acid sequence (SEQ ID NO: 152) derived from the coding sequence of SEQ ID NO: 151 shown in FIG. 151.

FIG. 153 shows a nucleotide sequence (SEQ ID NO: 153) of a native sequence PRO827 cDNA, wherein SEQ ID NO: 153 is a clone designated herein as “DNA57039-1402”.

FIG. 154 shows the amino acid sequence (SEQ ID NO: 154) derived from the coding sequence of SEQ ID NO: 153 shown in FIG. 153.

FIG. 155 shows a nucleotide sequence (SEQ ID NO: 155) of a native sequence PRO1057 cDNA, wherein SEQ ID NO: 155 is a clone designated herein as “DNA57253-1382”.

FIG. 156 shows the amino acid sequence (SEQ ID NO: 156) derived from the coding sequence of SEQ ID NO: 155 shown in FIG. 155.

FIG. 157 shows a nucleotide sequence (SEQ ID NO: 157) of a native sequence PRO 1113 cDNA, wherein SEQ ID NO: 157 is a clone designated herein as “DNA57254-1477”.

FIG. 158 shows the amino acid sequence (SEQ ID NO: 158) derived from the coding sequence of SEQ ID NO: 157 shown in FIG. 157.

FIG. 159 shows a nucleotide sequence (SEQ ID NO: 159) of a native sequence PRO1006 cDNA, wherein SEQ ID NO: 159 is a clone designated herein as “DNA57699-1412”.

FIG. 160 shows the amino acid sequence (SEQ ID NO: 160) derived from the coding sequence of SEQ ID NO: 159 shown in FIG. 159.

FIG. 161 shows a nucleotide sequence (SEQ ID NO: 161) of a native sequence PRO1074 cDNA, wherein SEQ ID NO: 161 is a clone designated herein as “DNA57704-1452”.

FIG. 162 shows the amino acid sequence (SEQ ID NO: 162) derived from the coding sequence of SEQ ID NO: 161 shown in FIG. 161.

FIG. 163 shows a nucleotide sequence (SEQ ID NO: 163) of a native sequence PRO1073 cDNA, wherein SEQ ID NO: 163 is a clone designated herein as “DNA57710-1451”.

FIG. 164 shows the amino acid sequence (SEQ ID NO: 164) derived from the coding sequence of SEQ ID NO: 163 shown in FIG. 163.

FIG. 165 shows a nucleotide sequence (SEQ ID NO: 165) of a native sequence PRO1136 cDNA, wherein SEQ ID NO: 165 is a clone designated herein as “DNA57827-1493”.

FIG. 166 shows the amino acid sequence (SEQ ID NO: 166) derived from the coding sequence of SEQ ID NO: 165 shown in FIG. 165.

FIG. 167 shows a nucleotide sequence (SEQ ID NO: 167) of a native sequence PRO1004 cDNA, wherein SEQ ID NO: 167 is a clone designated herein as “DNA57844-1410”.

FIG. 168 shows the amino acid sequence (SEQ ID NO: 168) derived from the coding sequence of SEQ ID NO: 167 shown in FIG. 167.

FIG. 169 shows a nucleotide sequence (SEQ ID NO: 169) of a native sequence PRO1344 cDNA, wherein SEQ ID NO: 169 is a clone designated herein as “DNA58723-1588”.

FIG. 170 shows the amino acid sequence (SEQ ID NO: 170) derived from the coding sequence of SEQ ID NO: 169 shown in FIG. 169.

FIG. 171 shows a nucleotide sequence (SEQ ID NO: 171) of a native sequence PRO 1110 cDNA, wherein SEQ ID NO: 171 is a clone designated herein as “DNA58727-1474”.

FIG. 172 shows the amino acid sequence (SEQ ID NO: 172) derived from the coding sequence of SEQ ID NO: 171 shown in FIG. 171.

FIG. 173 shows a nucleotide sequence (SEQ ID NO: 173) of a native sequence PRO1378 cDNA, wherein SEQ ID NO: 173 is a clone designated herein as “DNA58730-1607”.

FIG. 174 shows the amino acid sequence (SEQ ID NO: 174) derived from the coding sequence of SEQ ID NO: 173 shown in FIG. 173.

FIG. 175 shows a nucleotide sequence (SEQ ID NO: 175) of a native sequence PRO1481 cDNA, wherein SEQ ID NO: 175 is a clone designated herein as “DNA58732-1650”.

FIG. 176 shows the amino acid sequence (SEQ ID NO: 176) derived from the coding sequence of SEQ ID NO: 175 shown in FIG. 175.

FIG. 177 shows a nucleotide sequence (SEQ ID NO: 177) of a native sequence PRO1109 cDNA, wherein SEQ ID NO: 177 is a clone designated herein as “DNA58737-1473”.

FIG. 178 shows the amino acid sequence (SEQ ID NO: 178) derived from the coding sequence of SEQ ID NO: 177 shown in FIG. 177.

FIG. 179 shows a nucleotide sequence (SEQ ID NO: 179) of a native sequence PRO1383 cDNA, wherein SEQ ID NO: 179 is a clone designated herein as “DNA58743-1609”.

FIG. 180 shows the amino acid sequence (SEQ ID NO: 180) derived from the coding sequence of SEQ ID NO: 179 shown in FIG. 179.

FIG. 181 shows a nucleotide sequence (SEQ ID NO: 181) of a native sequence PRO1072 cDNA, wherein SEQ ID NO: 181 is a clone designated herein as “DNA58747-1384”.

FIG. 182 shows the amino acid sequence (SEQ ID NO: 182) derived from the coding sequence of SEQ ID NO: 181 shown in FIG. 181.

FIG. 183 shows a nucleotide sequence (SEQ ID NO: 183) of a native sequence PRO1189 cDNA, wherein SEQ ID NO: 183 is a clone designated herein as “DNA58828-1519”.

FIG. 184 shows the amino acid sequence (SEQ ID NO: 184) derived from the coding sequence of SEQ ID NO: 183 shown in FIG. 183.

FIG. 185 shows a nucleotide sequence (SEQ ID NO: 185) of a native sequence PRO1003 cDNA, wherein SEQ ID NO: 185 is a clone designated herein as “DNA58846-1409”.

FIG. 186 shows the amino acid sequence (SEQ ID NO: 186) derived from the coding sequence of SEQ ID NO: 185 shown in FIG. 185.

FIG. 187 shows a nucleotide sequence (SEQ ID NO: 187) of a native sequence PRO1108 cDNA, wherein SEQ ID NO: 187 is a clone designated herein as “DNA58848-1472”.

FIG. 188 shows the amino acid sequence (SEQ ID NO: 188) derived from the coding sequence of SEQ ID NO: 187 shown in FIG. 187.

FIG. 189 shows a nucleotide sequence (SEQ ID NO: 189) of a native sequence PRO1137 cDNA, wherein SEQ ID NO: 189 is a clone designated herein as “DNA58849-1494”.

FIG. 190 shows the amino acid sequence (SEQ ID NO: 190) derived from the coding sequence of SEQ ID NO: 189 shown in FIG. 189.

FIG. 191 shows a nucleotide sequence (SEQ ID NO: 191) of a native sequence PRO1138 cDNA, wherein SEQ ID NO: 191 is a clone designated herein as “DNA58850-1495”.

FIG. 192 shows the amino acid sequence (SEQ ID NO: 192) derived from the coding sequence of SEQ ID NO: 191 shown in FIG. 191.

FIG. 193 shows a nucleotide sequence (SEQ ID NO: 193) of a native sequence PRO 1415 cDNA, wherein SEQ ID NO: 193 is a clone designated herein as “DNA58852-1637”.

FIG. 194 shows the amino acid sequence (SEQ ID NO: 194) derived from the coding sequence of SEQ ID NO: 193 shown in FIG. 193.

FIG. 195 shows a nucleotide sequence (SEQ ID NO: 195) of a native sequence PRO 1054 cDNA, wherein SEQ ID NO: 195 is a clone designated herein as “DNA58853-1423”.

FIG. 196 shows the amino acid sequence (SEQ ID NO: 196) derived from the coding sequence of SEQ ID NO: 195 shown in FIG. 195.

FIG. 197 shows a nucleotide sequence (SEQ ID NO: 197) of a native sequence PRO994 cDNA, wherein SEQ ID NO: 197 is a clone designated herein as “DNA58855-1422”.

FIG. 198 shows the amino acid sequence (SEQ ID NO: 198) derived from the coding sequence of SEQ ID NO: 197 shown in FIG. 197.

FIG. 199 shows a nucleotide sequence (SEQ ID NO: 199) of a native sequence PRO 1069 cDNA, wherein SEQ ID NO: 199 is a clone designated herein as “DNA59211-1450”.

FIG. 200 shows the amino acid sequence (SEQ ID NO: 200) derived from the coding sequence of SEQ ID NO: 199 shown in FIG. 199.

FIG. 201 shows a nucleotide sequence (SEQ ID NO: 201) of a native sequence PRO1411 cDNA, wherein SEQ ID NO: 201 is a clone designated herein as “DNA59212-1627”.

FIG. 202 shows the amino acid sequence (SEQ ID NO: 202) derived from the coding sequence of SEQ ID NO: 201 shown in FIG. 201.

FIG. 203 shows a nucleotide sequence (SEQ ID NO: 203) of a native sequence PRO129 cDNA, wherein SEQ ID NO: 203 is a clone designated herein as “DNA59213-1487”.

FIG. 204 shows the amino acid sequence (SEQ ID NO: 204) derived from the coding sequence of SEQ ID NO: 203 shown in FIG. 203.

FIG. 205 shows a nucleotide sequence (SEQ ID NO: 205) of a native sequence PRO1359 cDNA, wherein SEQ ID NO: 205 is a clone designated herein as “DNA59219-1613”.

FIG. 206 shows the amino acid sequence (SEQ ID NO: 206) derived from the coding sequence of SEQ ID NO: 205 shown in FIG. 205.

FIG. 207 shows a nucleotide sequence (SEQ ID NO: 207) of a native sequence PRO1139 cDNA, wherein SEQ ID NO: 207 is a clone designated herein as “DNA59497-1496”.

FIG. 208 shows the amino acid sequence (SEQ ID NO: 208) derived from the coding sequence of SEQ ID NO: 207 shown in FIG. 207.

FIG. 209 shows a nucleotide sequence (SEQ ID NO: 209) of a native sequence PRO1065 cDNA, wherein SEQ ID NO: 209 is a clone designated herein as “DNA59602-1436”.

FIG. 210 shows the amino acid sequence (SEQ ID NO: 210) derived from the coding sequence of SEQ ID NO: 209 shown in FIG. 209.

FIG. 211 shows a nucleotide sequence (SEQ ID NO: 211) of a native sequence PRO 1028 cDNA, wherein SEQ ID NO: 211 is a clone designated herein as “DNA59603-1419”.

FIG. 212 shows the amino acid sequence (SEQ ID NO: 212) derived from the coding sequence of SEQ ID NO: 211 shown in FIG. 211.

FIG. 213 shows a nucleotide sequence (SEQ ID NO: 213) of a native sequence PRO1027 cDNA, wherein SEQ ID NO: 213 is a clone designated herein as “DNA59605-1418”.

FIG. 214 shows the amino acid sequence (SEQ ID NO: 214) derived from the coding sequence of SEQ ID NO: 213 shown in FIG. 213.

FIG. 215 shows a nucleotide sequence (SEQ ID NO: 215) of a native sequence PRO1140 cDNA, wherein SEQ ID NO: 215 is a clone designated herein as “DNA59607-1497”.

FIG. 216 shows the amino acid sequence (SEQ ID NO: 216) derived from the coding sequence of SEQ ID NO: 215 shown in FIG. 215.

FIG. 217 shows a nucleotide sequence (SEQ ID NO: 217) of a native sequence PRO1291 cDNA, wherein SEQ ID NO: 217 is a clone designated herein as “DNA59610-1556”.

FIG. 218 shows the amino acid sequence (SEQ ID NO: 218) derived from the coding sequence of SEQ ID NO: 217 shown in FIG. 217.

FIG. 219 shows a nucleotide sequence (SEQ ID NO: 219) of a native sequence PRO105 cDNA, wherein SEQ ID NO: 219 is a clone designated herein as “DNA59612-1466”.

FIG. 220 shows the amino acid sequence (SEQ ID NO: 220) derived from the coding sequence of SEQ ID NO: 219 shown in FIG. 219.

FIG. 221 shows a nucleotide sequence (SEQ ID NO: 221) of a native sequence PRO1026 cDNA, wherein SEQ ID NO: 221 is a clone designated herein as “DNA59613-1417”.

FIG. 222 shows the amino acid sequence (SEQ ID NO: 222) derived from the coding sequence of SEQ ID NO: 221 shown in FIG. 221.

FIG. 223 shows a nucleotide sequence (SEQ ID NO: 223) of a native sequence PRO104 cDNA, wherein SEQ ID NO: 223 is a clone designated herein as “DNA59616-1465”.

FIG. 224 shows the amino acid sequence (SEQ ID NO: 224) derived from the coding sequence of SEQ ID NO: 223 shown in FIG. 223.

FIG. 225 shows a nucleotide sequence (SEQ ID NO: 225) of a native sequence PRO1100 cDNA, wherein SEQ ID NO: 225 is a clone designated herein as “DNA59619-1464”.

FIG. 226 shows the amino acid sequence (SEQ ID NO: 226) derived from the coding sequence of SEQ ID NO: 225 shown in FIG. 225.

FIG. 227 shows a nucleotide sequence (SEQ ID NO: 227) of a native sequence PRO1141 cDNA, wherein SEQ ID NO: 227 is a clone designated herein as “DNA59625-1498”.

FIG. 228 shows the amino acid sequence (SEQ ID NO: 228) derived from the coding sequence of SEQ ID NO: 227 shown in FIG. 227.

FIG. 229 shows a nucleotide sequence (SEQ ID NO: 229) of a native sequence PRO1772 cDNA, wherein SEQ ID NO: 229 is a clone designated herein as “DNA59817-1703”.

FIG. 230 shows the amino acid sequence (SEQ ID NO: 230) derived from the coding sequence of SEQ ID NO: 229 shown in FIG. 229.

FIG. 231 shows a nucleotide sequence (SEQ ID NO: 231) of a native sequence PRO1064 cDNA, wherein SEQ ID NO: 231 is a clone designated herein as “DNA59827-1426”.

FIG. 232 shows the amino acid sequence (SEQ ID NO: 232) derived from the coding sequence of SEQ ID NO: 231 shown in FIG. 231.

FIG. 233 shows a nucleotide sequence (SEQ ID NO: 233) of a native sequence PRO1379 cDNA, wherein SEQ ID NO: 233 is a clone designated herein as “DNA59828-1608”.

FIG. 234 shows the amino acid sequence (SEQ ID NO: 234) derived from the coding sequence of SEQ ID NO: 233 shown in FIG. 233.

FIG. 235 shows a nucleotide sequence (SEQ ID NO: 235) of a native sequence PRO3573 cDNA, wherein SEQ ID NO: 235 is a clone designated herein as “DNA59837-2545”.

FIG. 236 shows the amino acid sequence (SEQ ID NO: 236) derived from the coding sequence of SEQ ID NO: 235 shown in FIG. 235.

FIG. 237 shows a nucleotide sequence (SEQ ID NO: 237) of a native sequence PRO3566 cDNA, wherein SEQ ID NO: 237 is a clone designated herein as “DNA59844-2542”.

FIG. 238 shows the amino acid sequence (SEQ ID NO: 238) derived from the coding sequence of SEQ ID NO: 237 shown in FIG. 237.

FIG. 239 shows a nucleotide sequence (SEQ ID NO: 239) of a native sequence PRO1156 cDNA, wherein SEQ ID NO: 239 is a clone designated herein as “DNA59853-1505”.

FIG. 240 shows the amino acid sequence (SEQ ID NO: 240) derived from the coding sequence of SEQ ID NO: 239 shown in FIG. 239.

FIG. 241 shows a nucleotide sequence (SEQ ID NO: 241) of a native sequence PRO1098 cDNA, wherein SEQ ID NO: 241 is a clone designated herein as “DNA59854-1459”.

FIG. 242 shows the amino acid sequence (SEQ ID NO: 242) derived from the coding sequence of SEQ ID NO: 241 shown in FIG. 241.

FIG. 243 shows a nucleotide sequence (SEQ ID NO: 243) of a native sequence PRO128 cDNA, wherein SEQ ID NO: 243 is a clone designated herein as “DNA59855-1485”.

FIG. 244 shows the amino acid sequence (SEQ ID NO: 244) derived from the coding sequence of SEQ ID NO: 243 shown in FIG. 243.

FIG. 245 shows a nucleotide sequence (SEQ ID NO: 245) of a native sequence PRO1248 cDNA, wherein SEQ ID NO: 245 is a clone designated herein as “DNA60278-1530”.

FIG. 246 shows the amino acid sequence (SEQ ID NO: 246) derived from the coding sequence of SEQ ID NO: 245 shown in FIG. 245.

FIG. 247 shows a nucleotide sequence (SEQ ID NO: 247) of a native sequence PRO1127 cDNA, wherein SEQ ID NO: 247 is a clone designated herein as “DNA60283-1484”.

FIG. 248 shows the amino acid sequence (SEQ ID NO: 248) derived from the coding sequence of SEQ ID NO: 247 shown in FIG. 247.

FIG. 249 shows a nucleotide sequence (SEQ ID NO: 249) of a native sequence PRO1316 cDNA, wherein SEQ ID NO: 249 is a clone designated herein as “DNA60608-1577”.

FIG. 250 shows the amino acid sequence (SEQ ID NO: 250) derived from the coding sequence of SEQ ID NO: 249 shown in FIG. 249.

FIG. 251 shows a nucleotide sequence (SEQ ID NO: 251) of a native sequence PRO197 cDNA, wherein SEQ ID NO: 251 is a clone designated herein as “DNA60611-1524”.

FIG. 252 shows the amino acid sequence (SEQ ID NO: 252) derived from the coding sequence of SEQ ID NO: 251 shown in FIG. 251.

FIG. 253 shows a nucleotide sequence (SEQ ID NO: 253) of a native sequence PRO1125 cDNA, wherein SEQ ID NO: 253 is a clone designated herein as “DNA60619-1482”.

FIG. 254 shows the amino acid sequence (SEQ ID NO: 254) derived from the coding sequence of SEQ ID NO: 253 shown in FIG. 253.

FIG. 255 shows a nucleotide sequence (SEQ ID NO: 255) of a native sequence PRO1158 cDNA, wherein SEQ ID NO: 255 is a clone designated herein as “DNA60625-1507”.

FIG. 256 shows the amino acid sequence (SEQ ID NO: 256) derived from the coding sequence of SEQ ID NO: 255 shown in FIG. 255.

FIG. 257 shows a nucleotide sequence (SEQ ID NO: 257) of a native sequence PRO124 cDNA, wherein SEQ ID NO: 257 is a clone designated herein as “DNA60629-1481”.

FIG. 258 shows the amino acid sequence (SEQ ID NO: 258) derived from the coding sequence of SEQ ID NO: 257 shown in FIG. 257.

FIG. 259 shows a nucleotide sequence (SEQ ID NO: 259) of a native sequence PRO1380 cDNA, wherein SEQ ID NO: 259 is a clone designated herein as “DNA60740-1615”.

FIG. 260 shows the amino acid sequence (SEQ ID NO: 260) derived from the coding sequence of SEQ ID NO: 259 shown in FIG. 259.

FIG. 261 shows a nucleotide sequence (SEQ ID NO: 261) of a native sequence PRO1377 cDNA, wherein SEQ ID NO: 261 is a clone designated herein as “DNA61608-1606”.

FIG. 262 shows the amino acid sequence (SEQ ID NO: 262) derived from the coding sequence of SEQ ID NO: 261 shown in FIG. 261.

FIG. 263 shows a nucleotide sequence (SEQ ID NO: 263) of a native sequence PRO1287 cDNA, wherein SEQ ID NO: 263 is a clone designated herein as “DNA61755-1554”.

FIG. 264 shows the amino acid sequence (SEQ ID NO: 264) derived from the coding sequence of SEQ ID NO: 263 shown in FIG. 263.

FIG. 265 shows a nucleotide sequence (SEQ ID NO: 265) of a native sequence PRO1249 cDNA, wherein SEQ ID NO: 265 is a clone designated herein as “DNA62809-1531”.

FIG. 266 shows the amino acid sequence (SEQ ID NO: 266) derived from the coding sequence of SEQ ID NO: 265 shown in FIG. 265.

FIG. 267 shows a nucleotide sequence (SEQ ID NO: 267) of a native sequence PRO1335 cDNA, wherein SEQ ID NO: 267 is a clone designated herein as “DNA62812-1594”.

FIG. 268 shows the amino acid sequence (SEQ ID NO: 268) derived from the coding sequence of SEQ ID NO: 267 shown in FIG. 267.

FIG. 269 shows a nucleotide sequence (SEQ ID NO: 269) of a native sequence PRO3572 cDNA, wherein SEQ ID NO: 269 is a clone designated herein as “DNA62813-2544”.

FIG. 270 shows the amino acid sequence (SEQ ID NO: 270) derived from the coding sequence of SEQ ID NO: 269 shown in FIG. 269.

FIG. 271 shows a nucleotide sequence (SEQ ID NO: 271) of a native sequence PRO1599 cDNA, wherein SEQ ID NO: 271 is a clone designated herein as “DNA62845-1684”.

FIG. 272 shows the amino acid sequence (SEQ ID NO: 272) derived from the coding sequence of SEQ ID NO: 271 shown in FIG. 271.

FIG. 273 shows a nucleotide sequence (SEQ ID NO: 273) of a native sequence PRO1374 cDNA, wherein SEQ ID NO: 273 is a clone designated herein as “DNA64849-1604”.

FIG. 274 shows the amino acid sequence (SEQ ID NO: 274) derived from the coding sequence of SEQ ID NO: 273 shown in FIG. 273.

FIG. 275 shows a nucleotide sequence (SEQ ID NO: 275) of a native sequence PRO1345 cDNA, wherein SEQ ID NO: 275 is a clone designated herein as “DNA64852-1589”.

FIG. 276 shows the amino acid sequence (SEQ ID NO: 276) derived from the coding sequence of SEQ ID NO: 275 shown in FIG. 275.

FIG. 277 shows a nucleotide sequence (SEQ ID NO: 277) of a native sequence PRO1311 cDNA, wherein SEQ ID NO: 277 is a clone designated herein as “DNA64863-1573”.

FIG. 278 shows the amino acid sequence (SEQ ID NO: 278) derived from the coding sequence of SEQ ID NO: 277 shown in FIG. 277.

FIG. 279 shows a nucleotide sequence (SEQ ID NO: 279) of a native sequence PRO1357 cDNA, wherein SEQ ID NO: 279 is a clone designated herein as “DNA64881-1602”.

FIG. 280 shows the amino acid sequence (SEQ ID NO: 280) derived from the coding sequence of SEQ ID NO: 279 shown in FIG. 279.

FIG. 281 shows a nucleotide sequence (SEQ ID NO: 281) of a native sequence PRO1557 cDNA, wherein SEQ ID NO: 281 is a clone designated herein as “DNA64902-1667”.

FIG. 282 shows the amino acid sequence (SEQ ID NO: 282) derived from the coding sequence of SEQ ID NO: 281 shown in FIG. 281.

FIG. 283 shows a nucleotide sequence (SEQ ID NO: 283) of a native sequence PRO1305 cDNA, wherein SEQ ID NO: 283 is a clone designated herein as “DNA64952-1568”.

FIG. 284 shows the amino acid sequence (SEQ ID NO: 284) derived from the coding sequence of SEQ ID NO: 283 shown in FIG. 283.

FIG. 285 shows a nucleotide sequence (SEQ ID NO: 285) of a native sequence PRO1302 cDNA, wherein SEQ ID NO: 285 is a clone designated herein as “DNA65403-1565”.

FIG. 286 shows the amino acid sequence (SEQ ID NO: 286) derived from the coding sequence of SEQ ID NO: 285 shown in FIG. 285.

FIG. 287 shows a nucleotide sequence (SEQ ID NO: 287) of a native sequence PRO1266 cDNA, wherein SEQ ID NO: 287 is a clone designated herein as “DNA65413-1534”.

FIG. 288 shows the amino acid sequence (SEQ ID NO: 288) derived from the coding sequence of SEQ ID NO: 287 shown in FIG. 287.

FIGS. 289A-289B show a nucleotide sequence (SEQ ID NO: 289) of a native sequence PRO1336 cDNA, wherein SEQ ID NO: 289 is a clone designated herein as “DNA65423-1595”.

FIG. 290 shows the amino acid sequence (SEQ ID NO: 290) derived from the coding sequence of SEQ ID NO: 289 shown in FIGS. 289A-289B.

FIG. 291 shows a nucleotide sequence (SEQ ID NO: 291) of a native sequence PRO1278 cDNA, wherein SEQ ID NO: 291 is a clone designated herein as “DNA66304-1546”.

FIG. 292 shows the amino acid sequence (SEQ ID NO: 292) derived from the coding sequence of SEQ ID NO: 291 shown in FIG. 291.

FIG. 293 shows a nucleotide sequence (SEQ ID NO: 293) of a native sequence PRO1270 cDNA, wherein SEQ ID NO: 293 is a clone designated herein as “DNA66308-1537”.

FIG. 294 shows the amino acid sequence (SEQ ID NO: 294) derived from the coding sequence of SEQ ID NO: 293 shown in FIG. 293.

FIG. 295 shows a nucleotide sequence (SEQ ID NO: 295) of a native sequence PRO1298 cDNA, wherein SEQ ID NO: 295 is a clone designated herein as “DNA66511-1563”.

FIG. 296 shows the amino acid sequence (SEQ ID NO: 296) derived from the coding sequence of SEQ ID NO: 295 shown in FIG. 295.

FIG. 297 shows a nucleotide sequence (SEQ ID NO: 297) of a native sequence PRO1301 cDNA, wherein SEQ ID NO: 297 is a clone designated herein as “DNA66512-1564”.

FIG. 298 shows the amino acid sequence (SEQ ID NO: 298) derived from the coding sequence of SEQ ID NO: 297 shown in FIG. 297.

FIG. 299 shows a nucleotide sequence (SEQ ID NO: 299) of a native sequence PRO1268 cDNA, wherein SEQ ID NO: 299 is a clone designated herein as “DNA66519-1535”.

FIG. 300 shows the amino acid sequence (SEQ ID NO: 300) derived from the coding sequence of SEQ ID NO: 299 shown in FIG. 299.

FIG. 301 shows a nucleotide sequence (SEQ ID NO: 301) of a native sequence PRO1327 cDNA, wherein SEQ ID NO: 301 is a clone designated herein as “DNA66521-1583”.

FIG. 302 shows the amino acid sequence (SEQ ID NO: 302) derived from the coding sequence of SEQ ID NO: 301 shown in FIG. 301.

FIG. 303 shows a nucleotide sequence (SEQ ID NO: 303) of a native sequence PRO1328 cDNA, wherein SEQ ID NO: 303 is a clone designated herein as “DNA66658-1584”.

FIG. 304 shows the amino acid sequence (SEQ ID NO: 304) derived from the coding sequence of SEQ ID NO: 303 shown in FIG. 303.

FIG. 305 shows a nucleotide sequence (SEQ ID NO: 305) of a native sequence PRO1329 cDNA, wherein SEQ ID NO: 305 is a clone designated herein as “DNA66660-1585”.

FIG. 306 shows the amino acid sequence (SEQ ID NO: 306) derived from the coding sequence of SEQ ID NO: 305 shown in FIG. 305.

FIG. 307 shows a nucleotide sequence (SEQ ID NO: 307) of a native sequence PRO1339 cDNA, wherein SEQ ID NO: 307 is a clone designated herein as “DNA66669-1597”.

FIG. 308 shows the amino acid sequence (SEQ ID NO: 308) derived from the coding sequence of SEQ ID NO: 307 shown in FIG. 307.

FIG. 309 shows a nucleotide sequence (SEQ ID NO: 309) of a native sequence PRO1342 cDNA, wherein SEQ ID NO: 309 is a clone designated herein as “DNA66674-1599”.

FIG. 310 shows the amino acid sequence (SEQ ID NO: 310) derived from the coding sequence of SEQ ID NO: 309 shown in FIG. 309.

FIGS. 311A-311B show a nucleotide sequence (SEQ ID NO: 311) of a native sequence PRO1487 cDNA, wherein SEQ ID NO: 311 is a clone designated herein as “DNA68836-1656”.

FIG. 312 shows the amino acid sequence (SEQ ID NO: 312) derived from the coding sequence of SEQ ID NO: 311 shown in FIGS. 311A-311B.

FIG. 313 shows a nucleotide sequence (SEQ ID NO: 313) of a native sequence PRO3579 cDNA, wherein SEQ ID NO: 313 is a clone designated herein as “DNA68862-2546”.

FIG. 314 shows the amino acid sequence (SEQ ID NO: 314) derived from the coding sequence of SEQ ID NO: 313 shown in FIG. 313.

FIG. 315 shows a nucleotide sequence (SEQ ID NO: 315) of a native sequence PRO1472 cDNA, wherein SEQ ID NO: 315 is a clone designated herein as “DNA68866-1644”.

FIG. 316 shows the amino acid sequence (SEQ ID NO: 316) derived from the coding sequence of SEQ ID NO: 315 shown in FIG. 315.

FIG. 317 shows a nucleotide sequence (SEQ ID NO: 317) of a native sequence PRO1385 cDNA, wherein SEQ ID NO: 317 is a clone designated herein as “DNA68869-1610”.

FIG. 318 shows the amino acid sequence (SEQ ID NO: 318) derived from the coding sequence of SEQ ID NO: 317 shown in FIG. 317.

FIG. 319 shows a nucleotide sequence (SEQ ID NO: 319) of a native sequence PRO1461 cDNA, wherein SEQ ID NO: 319 is a clone designated herein as “DNA68871-1638”.

FIG. 320 shows the amino acid sequence (SEQ ID NO: 320) derived from the coding sequence of SEQ ID NO: 319 shown in FIG. 319.

FIG. 321 shows a nucleotide sequence (SEQ ID NO: 321) of a native sequence PRO1429 cDNA, wherein SEQ ID NO: 321 is a clone designated herein as “DNA68879-1631”.

FIG. 322 shows the amino acid sequence (SEQ ID NO: 322) derived from the coding sequence of SEQ ID NO: 321 shown in FIG. 321.

FIG. 323 shows a nucleotide sequence (SEQ ID NO: 323) of a native sequence PRO1568 cDNA, wherein SEQ ID NO: 323 is a clone designated herein as “DNA68880-1676”.

FIG. 324 shows the amino acid sequence (SEQ ID NO: 324) derived from the coding sequence of SEQ ID NO: 323 shown in FIG. 323.

FIG. 325 shows a nucleotide sequence (SEQ ID NO: 325) of a native sequence PRO1569 cDNA, wherein SEQ ID NO: 325 is a clone designated herein as “DNA68882-1677”.

FIG. 326 shows the amino acid sequence (SEQ ID NO: 326) derived from the coding sequence of SEQ ID NO: 325 shown in FIG. 325.

FIG. 327 shows a nucleotide sequence (SEQ ID NO: 327) of a native sequence PRO1753 cDNA, wherein SEQ ID NO: 327 is a clone designated herein as “DNA68883-1691”.

FIG. 328 shows the amino acid sequence (SEQ ID NO: 328) derived from the coding sequence of SEQ ID NO: 327 shown in FIG. 327.

FIG. 329 shows a nucleotide sequence (SEQ ID NO: 329) of a native sequence PRO1570 cDNA, wherein SEQ ID NO: 329 is a clone designated herein as “DNA68885-1678”.

FIG. 330 shows the amino acid sequence (SEQ ID NO: 330) derived from the coding sequence of SEQ ID NO: 329 shown in FIG. 329.

FIG. 331 shows a nucleotide sequence (SEQ ID NO: 331) of a native sequence PRO1559 cDNA, wherein SEQ ID NO: 331 is a clone designated herein as “DNA68886”.

FIG. 332 shows the amino acid sequence (SEQ ID NO: 332) derived from the coding sequence of SEQ ID NO: 331 shown in FIG. 331.

FIG. 333 shows a nucleotide sequence (SEQ ID NO: 333) of a native sequence PRO1486 cDNA, wherein SEQ ID NO: 333 is a clone designated herein as “DNA71180-1655”.

FIG. 334 shows the amino acid sequence (SEQ ID NO: 334) derived from the coding sequence of SEQ ID NO: 333 shown in FIG. 333.

FIG. 335 shows a nucleotide sequence (SEQ ID NO: 335) of a native sequence PRO1433 cDNA, wherein SEQ ID NO: 335 is a clone designated herein as “DNA71184-1634”.

FIG. 336 shows the amino acid sequence (SEQ ID NO: 336) derived from the coding sequence of SEQ ID NO: 335 shown in FIG. 335.

FIG. 337 shows a nucleotide sequence (SEQ ID NO: 337) of a native sequence PRO1490 cDNA, wherein SEQ ID NO: 337 is a clone designated herein as “DNA71213-1659”.

FIG. 338 shows the amino acid sequence (SEQ ID NO: 338) derived from the coding sequence of SEQ ID NO: 337 shown in FIG. 337.

FIG. 339 shows a nucleotide sequence (SEQ ID NO: 339) of a native sequence PRO1482 cDNA, wherein SEQ ID NO: 339 is a clone designated herein as “DNA71234-1651”.

FIG. 340 shows the amino acid sequence (SEQ ID NO: 340) derived from the coding sequence of SEQ ID NO: 339 shown in FIG. 339.

FIG. 341 shows a nucleotide sequence (SEQ ID NO: 341) of a native sequence PRO1409 cDNA, wherein SEQ ID NO: 341 is a clone designated herein as “DNA71269-1621”.

FIG. 342 shows the amino acid sequence (SEQ ID NO: 342) derived from the coding sequence of SEQ ID NO: 341 shown in FIG. 341.

FIG. 343 shows a nucleotide sequence (SEQ ID NO: 343) of a native sequence PRO1446 cDNA, wherein SEQ ID NO: 343 is a clone designated herein as “DNA71277-1636”.

FIG. 344 shows the amino acid sequence (SEQ ID NO: 344) derived from the coding sequence of SEQ ID NO: 343 shown in FIG. 343.

FIG. 345 shows a nucleotide sequence (SEQ ID NO: 345) of a native sequence PRO1604 cDNA, wherein SEQ ID NO: 345 is a clone designated herein as “DNA71286-1687”.

FIG. 346 shows the amino acid sequence (SEQ ID NO: 346) derived from the coding sequence of SEQ ID NO: 345 shown in FIG. 345.

FIG. 347 shows a nucleotide sequence (SEQ ID NO: 347) of a native sequence PRO1491 cDNA, wherein SEQ ID NO: 347 is a clone designated herein as “DNA71883-1660”.

FIG. 348 shows the amino acid sequence (SEQ ID NO: 348) derived from the coding sequence of SEQ ID NO: 347 shown in FIG. 347.

FIG. 349 shows a nucleotide sequence (SEQ ID NO: 349) of a native sequence PRO1431 cDNA, wherein SEQ ID NO: 349 is a clone designated herein as “DNA73401-1633”.

FIG. 350 shows the amino acid sequence (SEQ ID NO: 350) derived from the coding sequence of SEQ ID NO: 349 shown in FIG. 349.

FIGS. 351A-351B show a nucleotide sequence (SEQ ID NO: 351) of a native sequence PRO1563 cDNA, wherein SEQ ID NO: 351 is a clone designated herein as “DNA73492-1671”.

FIG. 352 shows the amino acid sequence (SEQ ID NO: 352) derived from the coding sequence of SEQ ID NO: 351 shown in FIGS. 351A-351B.

FIG. 353 shows a nucleotide sequence (SEQ ID NO: 353) of a native sequence PRO1571 cDNA, wherein SEQ ID NO: 353 is a clone designated herein as “DNA73730-1679”.

FIG. 354 shows the amino acid sequence (SEQ ID NO: 354) derived from the coding sequence of SEQ ID NO: 353 shown in FIG. 353.

FIG. 355 shows a nucleotide sequence (SEQ ID NO: 355) of a native sequence PRO1572 cDNA, wherein SEQ ID NO: 355 is a clone designated herein as “DNA73734-1680”.

FIG. 356 shows the amino acid sequence (SEQ ID NO: 356) derived from the coding sequence of SEQ ID NO: 355 shown in FIG. 355.

FIG. 357 shows a nucleotide sequence (SEQ ID NO: 357) of a native sequence PRO1573 cDNA, wherein SEQ ID NO: 357 is a clone designated herein as “DNA73735-1681”.

FIG. 358 shows the amino acid sequence (SEQ ID NO: 358) derived from the coding sequence of SEQ ID NO: 357 shown in FIG. 357.

FIG. 359 shows a nucleotide sequence (SEQ ID NO: 359) of a native sequence PRO1508 cDNA, wherein SEQ ID NO: 359 is a clone designated herein as “DNA73742-1662”.

FIG. 360 shows the amino acid sequence (SEQ ID NO: 360) derived from the coding sequence of SEQ ID NO: 359 shown in FIG. 359.

FIG. 361 shows a nucleotide sequence (SEQ ID NO: 361) of a native sequence PRO1485 cDNA, wherein SEQ ID NO: 361 is a clone designated herein as “DNA73746-1654”.

FIG. 362 shows the amino acid sequence (SEQ ID NO: 362) derived from the coding sequence of SEQ ID NO: 361 shown in FIG. 361.

FIG. 363 shows a nucleotide sequence (SEQ ID NO: 363) of a native sequence PRO1564 cDNA, wherein SEQ ID NO: 363 is a clone designated herein as “DNA73760-1672”.

FIG. 364 shows the amino acid sequence (SEQ ID NO: 364) derived from the coding sequence of SEQ ID NO: 363 shown in FIG. 363.

FIG. 365 shows a nucleotide sequence (SEQ ID NO: 365) of a native sequence PRO1550 cDNA, wherein SEQ ID NO: 365 is a clone designated herein as “DNA76393-1664”.

FIG. 366 shows the amino acid sequence (SEQ ID NO: 366) derived from the coding sequence of SEQ ID NO: 365 shown in FIG. 365.

FIG. 367 shows a nucleotide sequence (SEQ ID NO: 367) of a native sequence PRO1757 cDNA, wherein SEQ ID NO: 367 is a clone designated herein as “DNA76398-1699”.

FIG. 368 shows the amino acid sequence (SEQ ID NO: 368) derived from the coding sequence of SEQ ID NO: 367 shown in FIG. 367.

FIG. 369 shows a nucleotide sequence (SEQ ID NO: 369) of a native sequence PRO1758 cDNA, wherein SEQ ID NO: 369 is a clone designated herein as “DNA76399-1700”.

FIG. 370 shows the amino acid sequence (SEQ ID NO: 370) derived from the coding sequence of SEQ ID NO: 369 shown in FIG. 369.

FIG. 371 shows a nucleotide sequence (SEQ ID NO: 371) of a native sequence PRO1781 cDNA, wherein SEQ ID NO: 371 is a clone designated herein as “DNA76522-2500”.

FIG. 372 shows the amino acid sequence (SEQ ID NO: 372) derived from the coding sequence of SEQ ID NO: 371 shown in FIG. 371.

FIG. 373 shows a nucleotide sequence (SEQ ID NO: 373) of a native sequence PRO1606 cDNA, wherein SEQ ID NO: 373 is a clone designated herein as “DNA76533-1689”.

FIG. 374 shows the amino acid sequence (SEQ ID NO: 374) derived from the coding sequence of SEQ ID NO: 373 shown in FIG. 373.

FIG. 375 shows a nucleotide sequence (SEQ ID NO: 375) of a native sequence PRO1784 cDNA, wherein SEQ ID NO: 375 is a clone designated herein as “DNA77303-2502”.

FIG. 376 shows the amino acid sequence (SEQ ID NO: 376) derived from the coding sequence of SEQ ID NO: 375 shown in FIG. 375.

FIG. 377 shows a nucleotide sequence (SEQ ID NO: 377) of a native sequence PRO1774 cDNA, wherein SEQ ID NO: 377 is a clone designated herein as “DNA77626-1705”.

FIG. 378 shows the amino acid sequence (SEQ ID NO: 378) derived from the coding sequence of SEQ ID NO: 377 shown in FIG. 377.

FIG. 379 shows a nucleotide sequence (SEQ ID NO: 379) of a native sequence PRO1605 cDNA, wherein SEQ ID NO: 379 is a clone designated herein as “DNA77648-1688”.

FIG. 380 shows the amino acid sequence (SEQ ID NO: 380) derived from the coding sequence of SEQ ID NO: 379 shown in FIG. 379.

FIG. 381 shows a nucleotide sequence (SEQ ID NO: 381) of a native sequence PRO1928 cDNA, wherein SEQ ID NO: 381 is a clone designated herein as “DNA81754-2532”.

FIG. 382 shows the amino acid sequence (SEQ ID NO: 382) derived from the coding sequence of SEQ ID NO: 381 shown in FIG. 381.

FIG. 383 shows a nucleotide sequence (SEQ ID NO: 383) of a native sequence PRO1865 cDNA, wherein SEQ ID NO: 383 is a clone designated herein as “DNA81757-2512”.

FIG. 384 shows the amino acid sequence (SEQ ID NO: 384) derived from the coding sequence of SEQ ID NO: 383 shown in FIG. 383.

FIG. 385 shows a nucleotide sequence (SEQ ID NO: 385) of a native sequence PRO1925 cDNA,wherein SEQ ID NO: 385 is a clone designated herein as “DNA82302-2529”.

FIG. 386 shows the amino acid sequence (SEQ ID NO: 386) derived from the coding sequence of SEQ ID NO: 385 shown in FIG. 385.

FIG. 387 shows a nucleotide sequence (SEQ ID NO: 387) of a native sequence PRO1926 cDNA, wherein SEQ ID NO: 387 is a clone designated herein as “DNA82340-2530”.

FIG. 388 shows the amino acid sequence (SEQ ID NO: 388) derived from the coding sequence of SEQ ID NO: 387 shown in FIG. 387.

FIG. 389 shows a nucleotide sequence (SEQ ID NO: 389) of a native sequence PRO2630 cDNA, wherein SEQ ID NO: 389 is a clone designated herein as “DNA83551”.

FIG. 390 shows the amino acid sequence (SEQ ID NO: 390) derived from the coding sequence of SEQ ID NO: 389 shown in FIG. 389.

FIG. 391 shows a nucleotide sequence (SEQ ID NO: 391) of a native sequence PRO3443 cDNA, wherein SEQ ID NO: 391 is a clone designated herein as “DNA87991-2540”.

FIG. 392 shows the amino acid sequence (SEQ ID NO: 392) derived from the coding sequence of SEQ ID NO: 391 shown in FIG. 391.

FIG. 393 shows a nucleotide sequence (SEQ ID NO: 393) of a native sequence PRO3301 cDNA, wherein SEQ ID NO: 393 is a clone designated herein as “DNA88002”.

FIG. 394 shows the amino acid sequence (SEQ ID NO: 394) derived from the coding sequence of SEQ ID NO: 393 shown in FIG. 393.

FIG. 395 shows a nucleotide sequence (SEQ ID NO: 395) of a native sequence PRO3442 cDNA, wherein SEQ ID NO: 395 is a clone designated herein as “DNA92238-2539”.

FIG. 396 shows the amino acid sequence (SEQ ID NO: 396) derived from the coding sequence of SEQ ID NO: 395 shown in FIG. 395.

FIG. 397 shows a nucleotide sequence (SEQ ID NO: 397) of a native sequence PRO4978 cDNA, wherein SEQ ID NO: 397 is a clone designated herein as “DNA95930”.

FIG. 398 shows the amino acid sequence (SEQ ID NO: 398) derived from the coding sequence of SEQ ID NO: 397 shown in FIG. 397.

FIG. 399 shows a nucleotide sequence (SEQ ID NO: 399) of a native sequence PRO5801 cDNA, wherein SEQ ID NO: 399 is a clone designated herein as “DNA115291-2681”.

FIG. 400 shows the amino acid sequence (SEQ ID NO: 400) derived from the coding sequence of SEQ ID NO: 399 shown in FIG. 399.

FIG. 401 shows a nucleotide sequence (SEQ ID NO: 401) of a native sequence PRO19630 cDNA, wherein SEQ ID NO: 401 is a clone designated herein as “DNA23336-2861”.

FIG. 402 shows the amino acid sequence (SEQ ID NO: 402) derived from the coding sequence of SEQ ID NO: 401 shown in FIG. 401.

FIG. 403 shows a nucleotide sequence (SEQ ID NO: 403) of a native sequence PRO203 cDNA, wherein SEQ ID NO: 403 is a clone designated herein as “DNA30862-1396”.

FIG. 404 shows the amino acid sequence (SEQ ID NO: 404) derived from the coding sequence of SEQ ID NO: 403 shown in FIG. 403.

FIG. 405 shows a nucleotide sequence (SEQ ID NO: 405) of a native sequence PRO204 cDNA, wherein SEQ ID NO: 405 is a clone designated herein as “DNA30871-1157”.

FIG. 406 shows the amino acid sequence (SEQ ID NO: 406) derived from the coding sequence of SEQ ID NO: 405 shown in FIG. 405.

FIG. 407 shows a nucleotide sequence (SEQ ID NO: 407) of a native sequence PRO210 cDNA, wherein SEQ ID NO: 407 is a clone designated herein as “DNA32279-1131”.

FIG. 408 shows the amino acid sequence (SEQ ID NO: 408) derived from the coding sequence of SEQ ID NO: 407 shown in FIG. 407.

FIG. 409 shows a nucleotide sequence (SEQ ID NO: 409) of a native sequence PRO223 cDNA, wherein SEQ ID NO: 409 is a clone designated herein as “DNA33206-1165”.

FIG. 410 shows the amino acid sequence (SEQ ID NO: 410) derived from the coding sequence of SEQ ID NO: 409 shown in FIG. 409.

FIG. 411 shows a nucleotide sequence (SEQ ID NO: 411) of a native sequence PRO247 cDNA, wherein SEQ ID NO: 411 is a clone designated herein as “DNA35673-1201”.

FIG. 412 shows the amino acid sequence (SEQ ID NO: 412) derived from the coding sequence of SEQ ID NO: 411 shown in FIG. 411.

FIG. 413 shows a nucleotide sequence (SEQ ID NO: 413) of a native sequence PRO358 cDNA, wherein SEQ ID NO: 413 is a clone designated herein as “DNA47361-1154-2”.

FIG. 414 shows the amino acid sequence (SEQ ID NO: 414) derived from the coding sequence of SEQ ID NO: 413 shown in FIG. 413.

FIG. 415 shows a nucleotide sequence (SEQ ID NO: 415) of a native sequence PRO724 cDNA, wherein SEQ ID NO: 415 is a clone designated herein as “DNA49631-1328”.

FIG. 416 shows the amino acid sequence (SEQ ID NO: 416) derived from the coding sequence of SEQ ID NO: 415 shown in FIG. 415.

FIG. 417 shows a nucleotide sequence (SEQ ID NO: 417) of a native sequence PRO868 cDNA, wherein SEQ ID NO: 417 is a clone designated herein as “DNA52594-1270”.

FIG. 418 shows the amino acid sequence (SEQ ID NO: 418) derived from the coding sequence of SEQ ID NO: 417 shown in FIG. 417.

FIG. 419 shows a nucleotide sequence (SEQ ID NO: 419) of a native sequence PRO740 cDNA, wherein SEQ ID NO: 419 is a clone designated herein as “DNA55800-1263”.

FIG. 420 shows the amino acid sequence (SEQ ID NO: 420) derived from the coding sequence of SEQ ID NO: 419 shown in FIG. 419.

FIG. 421 shows a nucleotide sequence (SEQ ID NO: 421) of a native sequence PRO1478 cDNA, wherein SEQ ID NO: 421 is a clone designated herein as “DNA56531-1648”.

FIG. 422 shows the amino acid sequence (SEQ ID NO: 422) derived from the coding sequence of SEQ ID NO: 421 shown in FIG. 421.

FIG. 423 shows a nucleotide sequence (SEQ ID NO: 423) of a native sequence PRO162 cDNA, wherein SEQ ID NO: 423 is a clone designated herein as “DNA56965-1356”.

FIG. 424 shows the amino acid sequence (SEQ ID NO: 424) derived from the coding sequence of SEQ ID NO: 423 shown in FIG. 423.

FIG. 425 shows a nucleotide sequence (SEQ ID NO: 425) of a native sequence PRO828 cDNA, wherein SEQ ID NO: 425 is a clone designated herein as “DNA57037-1444”.

FIG. 426 shows the amino acid sequence (SEQ ID NO: 426) derived from the coding sequence of SEQ ID NO: 425 shown in FIG. 425.

FIG. 427 shows a nucleotide sequence (SEQ ID NO: 427) of a native sequence PRO819 cDNA, wherein SEQ ID NO: 427 is a clone designated herein as “DNA57695-1340”.

FIG. 428 shows the amino acid sequence (SEQ ID NO: 428) derived from the coding sequence of SEQ ID NO: 427 shown in FIG. 427.

FIG. 429 shows a nucleotide sequence (SEQ ID NO: 429) of a native sequence PRO813 cDNA, wherein SEQ ID NO: 429 is a clone designated herein as “DNA57834-1339”.

FIG. 430 shows the amino acid sequence (SEQ ID NO: 430) derived from the coding sequence of SEQ ID NO: 429 shown in FIG. 429.

FIG. 431 shows a nucleotide sequence (SEQ ID NO: 43 1) of a native sequence PRO 1194 cDNA, wherein SEQ ID NO: 431 is a clone designated herein as “DNA57841-1522”.

FIG. 432 shows the amino acid sequence (SEQ ID NO: 432) derived from the coding sequence of SEQ ID NO: 431 shown in FIG. 431.

FIG. 433 shows a nucleotide sequence (SEQ ID NO: 433) of a native sequence PRO887 cDNA, wherein SEQ ID NO: 433 is a clone designated herein as “DNA58130”.

FIG. 434 shows the amino acid sequence (SEQ ID NO: 434) derived from the coding sequence of SEQ ID NO: 433 shown in FIG. 433.

FIG. 435 shows a nucleotide sequence (SEQ ID NO: 435) of a native sequence PRO1071 cDNA, wherein SEQ ID NO: 435 is a clone designated herein as “DNA58847-1383”.

FIG. 436 shows the amino acid sequence (SEQ ID NO: 436) derived from the coding sequence of SEQ ID NO: 435 shown in FIG. 435.

FIG. 437 shows a nucleotide sequence (SEQ ID NO: 437) of a native sequence PRO 1029 cDNA, wherein SEQ ID NO: 437 is a clone designated herein as “DNA59493-1420”.

FIG. 438 shows the amino acid sequence (SEQ ID NO: 438) derived from the coding sequence of SEQ ID NO: 437 shown in FIG. 437.

FIG. 439 shows a nucleotide sequence (SEQ ID NO: 439) of a native sequence PRO1190 cDNA, wherein SEQ ID NO: 439 is a clone designated herein as “DNA59586-1520”.

FIG. 440 shows the amino acid sequence (SEQ ID NO: 440) derived from the coding sequence of SEQ ID NO: 439 shown in FIG. 439.

FIG. 441 shows a nucleotide sequence (SEQ ID NO: 441) of a native sequence PRO4334 cDNA, wherein SEQ ID NO: 441 is a clone designated herein as “DNA59608-2577”.

FIG. 442 shows the amino acid sequence (SEQ ID NO: 442) derived from the coding sequence of SEQ ID NO: 441 shown in FIG. 441.

FIG. 443 shows a nucleotide sequence (SEQ ID NO: 443) of a native sequence PRO1155 cDNA, wherein SEQ ID NO: 443 is a clone designated herein as “DNA59849-1504”.

FIG. 444 shows the amino acid sequence (SEQ ID NO: 444) derived from the coding sequence of SEQ ID NO: 443 shown in FIG. 443.

FIG. 445 shows a nucleotide sequence (SEQ ID NO: 445) of a native sequence PRO1157 cDNA, wherein SEQ ID NO: 445 is a clone designated herein as “DNA60292-1506”.

FIG. 446 shows the amino acid sequence (SEQ ID NO: 446) derived from the coding sequence of SEQ ID NO: 445 shown in FIG. 445.

FIG. 447 shows a nucleotide sequence (SEQ ID NO: 447) of a native sequence PRO 1122 cDNA, wherein SEQ ID NO: 447 is a clone designated herein as “DNA62377-1381-1”.

FIG. 448 shows the amino acid sequence (SEQ ID NO: 448) derived from the coding sequence of SEQ ID NO: 447 shown in FIG. 447.

FIG. 449 shows a nucleotide sequence (SEQ ID NO: 449) of a native sequence PRO 1183 cDNA, wherein SEQ ID NO: 449 is a clone designated herein as “DNA62880-1513”.

FIG. 450 shows the amino acid sequence (SEQ ID NO: 450) derived from the coding sequence of SEQ ID NO: 449 shown in FIG. 449.

FIG. 451 shows a nucleotide sequence (SEQ ID NO: 451) of a native sequence PRO1337 cDNA, wherein SEQ ID NO: 451 is a clone designated herein as “DNA66672-1586”.

FIG. 452 shows the amino acid sequence (SEQ ID NO: 452) derived from the coding sequence of SEQ ID NO: 451 shown in FIG. 451.

FIG. 453 shows a nucleotide sequence (SEQ ID NO: 453) of a native sequence PRO1480 cDNA, wherein SEQ ID NO: 453 is a clone designated herein as “DNA67962-1649”.

FIG. 454 shows the amino acid sequence (SEQ ID NO: 454) derived from the coding sequence of SEQ ID NO: 453 shown in FIG. 453.

FIG. 455 shows a nucleotide sequence (SEQ ID NO: 455) of a native sequence PRO19645 cDNA, wherein SEQ ID NO: 455 is a clone designated herein as “DNA69555-2867”.

FIG. 456 shows the amino acid sequence (SEQ ID NO: 456) derived from the coding sequence of SEQ ID NO: 455 shown in FIG. 455.

FIG. 457 shows a nucleotide sequence (SEQ ID NO: 457) of a native sequence PRO9782 cDNA, wherein SEQ ID NO: 457 is a clone designated herein as “DNA71162-2764”.

FIG. 458 shows the amino acid sequence (SEQ ID NO: 458) derived from the coding sequence of SEQ ID NO: 457 shown in FIG. 457.

FIG. 459 shows a nucleotide sequence (SEQ ID NO: 459) of a native sequence PRO1419 cDNA, wherein SEQ ID NO: 459 is a clone designated herein as “DNA71290-1630”.

FIG. 460 shows the amino acid sequence (SEQ ID NO: 460) derived from the coding sequence of SEQ ID NO: 459 shown in FIG. 459.

FIG. 461 shows a nucleotide sequence (SEQ ID NO: 461) of a native sequence PRO1575 cDNA, wherein SEQ ID NO: 461 is a clone designated herein as “DNA76401-1683”.

FIG. 462 shows the amino acid sequence (SEQ ID NO: 462) derived from the coding sequence of SEQ ID NO: 461 shown in FIG. 461.

FIG. 463 shows a nucleotide sequence (SEQ ID NO: 463) of a native sequence PRO1567 cDNA, wherein SEQ ID NO: 463 is a clone designated herein as “DNA76541-1675”.

FIG. 464 shows the amino acid sequence (SEQ ID NO: 464) derived from the coding sequence of SEQ ID NO: 463 shown in FIG. 463.

FIG. 465 shows a nucleotide sequence (SEQ ID NO: 465) of a native sequence PRO1891 cDNA, wherein SEQ ID NO: 465 is a clone designated herein as “DNA76788-2526”.

FIG. 466 shows the amino acid sequence (SEQ ID NO: 466) derived from the coding sequence of SEQ ID NO: 465 shown in FIG. 465.

FIG. 467 shows a nucleotide sequence (SEQ ID NO: 467) of a native sequence PRO1889 cDNA, wherein SEQ ID NO: 467 is a clone designated herein as “DNA77623-2524”.

FIG. 468 shows the amino acid sequence (SEQ ID NO: 468) derived from the coding sequence of SEQ ID NO: 467 shown in FIG. 467.

FIG. 469 shows a nucleotide sequence (SEQ ID NO: 469) of a native sequence PRO1785 cDNA, wherein SEQ ID NO: 469 is a clone designated herein as “DNA80136-2503”.

FIG. 470 shows the amino acid sequence (SEQ ID NO: 470) derived from the coding sequence of SEQ ID NO: 469 shown in FIG. 469.

FIG. 471 shows a nucleotide sequence (SEQ ID NO: 471) of a native sequence PRO6003 cDNA, wherein SEQ ID NO: 471 is a clone designated herein as “DNA83568-2692”.

FIG. 472 shows the amino acid sequence (SEQ ID NO: 472) derived from the coding sequence of SEQ ID NO: 471 shown in FIG. 471.

FIG. 473 shows a nucleotide sequence (SEQ ID NO: 473) of a native sequence PRO4333 cDNA, wherein SEQ ID NO: 473 is a clone designated herein as “DNA84210-2576”.

FIG. 474 shows the amino acid sequence (SEQ ID NO: 474) derived from the coding sequence of SEQ ID NO: 473 shown in FIG. 473.

FIG. 475 shows a nucleotide sequence (SEQ ID NO: 475) of a native sequence PRO4356 cDNA,wherein SEQ ID NO: 475 is a clone designated herein as “DNA86576-2595”.

FIG. 476 shows the amino acid sequence (SEQ ID NO: 476) derived from the coding sequence of SEQ ID NO: 475 shown in FIG. 475.

FIG. 477 shows a nucleotide sequence (SEQ ID NO: 477) of a native sequence PRO4352 cDNA, wherein SEQ ID NO: 477 is a clone designated herein as “DNA87976-2593”.

FIG. 478 shows the amino acid sequence (SEQ ID NO: 478) derived from the coding sequence of SEQ ID NO: 477 shown in FIG. 477.

FIG. 479 shows a nucleotide sequence (SEQ ID NO: 479) of a native sequence PRO4354 cDNA, wherein SEQ ID NO: 479 is a clone designated herein as “DNA92256-2596”.

FIG. 480 shows the amino acid sequence (SEQ ID NO: 480) derived from the coding sequence of SEQ ID NO: 479 shown in FIG. 479.

FIG. 481 shows a nucleotide sequence (SEQ ID NO: 481) of a native sequence PRO4369 cDNA, wherein SEQ ID NO: 481 is a clone designated herein as “DNA92289-2598”.

FIG. 482 shows the amino acid sequence (SEQ ID NO: 482) derived from the coding sequence of SEQ ID NO: 481 shown in FIG. 481.

FIG. 483 shows a nucleotide sequence (SEQ ID NO: 483) of a native sequence PRO6030 cDNA, wherein SEQ ID NO: 483 is a clone designated herein as “DNA96850-2705”.

FIG. 484 shows the amino acid sequence (SEQ ID NO: 484) derived from the coding sequence of SEQ ID NO: 483 shown in FIG. 483.

FIG. 485 shows a nucleotide sequence (SEQ ID NO: 485) of a native sequence PRO4433 cDNA, wherein SEQ ID NO: 485 is a clone designated herein as “DNA96855-2629”.

FIG. 486 shows the amino acid sequence (SEQ ID NO: 486) derived from the coding sequence of SEQ ID NO: 485 shown in FIG. 485.

FIG. 487 shows a nucleotide sequence (SEQ ID NO: 487) of a native sequence PRO4424 cDNA,wherein SEQ ID NO: 487 is a clone designated herein as “DNA96857-2636”.

FIG. 488 shows the amino acid sequence (SEQ ID NO: 488) derived from the coding sequence of SEQ ID NO: 487 shown in FIG. 487.

FIG. 489 shows a nucleotide sequence (SEQ ID NO: 489) of a native sequence PRO6017 cDNA, wherein SEQ ID NO: 489 is a clone designated herein as “DNA96860-2700”.

FIG. 490 shows the amino acid sequence (SEQ ID NO: 490) derived from the coding sequence of SEQ ID NO: 489 shown in FIG. 489.

FIG. 491 shows a nucleotide sequence (SEQ ID NO: 491) of a native sequence PRO19563 cDNA, wherein SEQ ID NO: 491 is a clone designated herein as “DNA96861-2844”.

FIG. 492 shows the amino acid sequence (SEQ ID NO: 492) derived from the coding sequence of SEQ ID NO: 491 shown in FIG. 491.

FIG. 493 shows a nucleotide sequence (SEQ ID NO: 493) of a native sequence PRO6015 cDNA,wherein SEQ ID NO: 493 is a clone designated herein as “DNA96866-2698”.

FIG. 494 shows the amino acid sequence (SEQ ID NO: 494) derived from the coding sequence of SEQ ID NO: 493 shown in FIG. 493.

FIG. 495 shows a nucleotide sequence (SEQ ID NO: 495) of a native sequence PRO5779 cDNA, wherein SEQ ID NO: 495 is a clone designated herein as “DNA96870-2676”.

FIG. 496 shows the amino acid sequence (SEQ ID NO: 496) derived from the coding sequence of SEQ ID NO: 495 shown in FIG. 495.

FIG. 497 shows a nucleotide sequence (SEQ ID NO: 497) of a native sequence PRO5776 cDNA, wherein SEQ ID NO: 497 is a clone designated herein as “DNA96872-2674”.

FIG. 498 shows the amino acid sequence (SEQ ID NO: 498) derived from the coding sequence of SEQ ID NO: 497 shown in FIG. 497.

FIG. 499 shows a nucleotide sequence (SEQ ID NO: 499) of a native sequence PRO4430 cDNA, wherein SEQ ID NO: 499 is a clone designated herein as “DNA96878-2626”.

FIG. 500 shows the amino acid sequence (SEQ ID NO: 500) derived from the coding sequence of SEQ ID NO: 499 shown in FIG. 499.

FIG. 501 shows a nucleotide sequence (SEQ ID NO: 501) of a native sequence PRO4421 cDNA, wherein SEQ ID NO: 501 is a clone designated herein as “DNA96879-2619”.

FIG. 502 shows the amino acid sequence (SEQ ID NO: 502) derived from the coding sequence of SEQ ID NO: 501 shown in FIG. 501.

FIG. 503 shows a nucleotide sequence (SEQ ID NO: 503) of a native sequence PRO4499 cDNA, wherein SEQ ID NO: 503 is a clone designated herein as “DNA96889-2641”.

FIG. 504 shows the amino acid sequence (SEQ ID NO: 504) derived from the coding sequence of SEQ ID NO: 503 shown in FIG. 503.

FIG. 505 shows a nucleotide sequence (SEQ ID NO: 505) of a native sequence PRO4423 cDNA, wherein SEQ ID NO: 505 is a clone designated herein as “DNA96893-2621”.

FIG. 506 shows the amino acid sequence (SEQ ID NO: 506) derived from the coding sequence of SEQ ID NO: 505 shown in FIG. 505.

FIG. 507 shows a nucleotide sequence (SEQ ID NO: 507) of a native sequence PRO5998 cDNA, wherein SEQ ID NO: 507 is a clone designated herein as “DNA96897-2688”.

FIG. 508 shows the amino acid sequence (SEQ ID NO: 508) derived from the coding sequence of SEQ ID NO: 507 shown in FIG. 507.

FIG. 509 shows a nucleotide sequence (SEQ ID NO: 509) of a native sequence PRO4501 cDNA, wherein SEQ ID NO: 509 is a clone designated herein as “DNA98564-2643”.

FIG. 510 shows the amino acid sequence (SEQ ID NO: 510) derived from the coding sequence of SEQ ID NO: 509 shown in FIG. 509.

FIG. 511 shows a nucleotide sequence (SEQ ID NO: 51 1) of a native sequence PRO6240 cDNA, wherein SEQ ID NO: 511 is a clone designated herein as “DNA107443-2718”.

FIG. 512 shows the amino acid sequence (SEQ ID NO: 512) derived from the coding sequence of SEQ ID NO: 511 shown in FIG. 511.

FIG. 513 shows a nucleotide sequence (SEQ ID NO: 513) of a native sequence PRO6245 cDNA, wherein SEQ ID NO: 513 is a clone designated herein as “DNA107786-2723”.

FIG. 514 shows the amino acid sequence (SEQ ID NO: 514) derived from the coding sequence of SEQ ID NO: 513 shown in FIG. 513.

FIG. 515 shows a nucleotide sequence (SEQ ID NO: 515) of a native sequence PRO6175 cDNA, wherein SEQ ID NO: 515 is a clone designated herein as “DNA108682-2712”.

FIG. 516 shows the amino acid sequence (SEQ ID NO: 516) derived from the coding sequence of SEQ ID NO: 515 shown in FIG. 515.

FIG. 517 shows a nucleotide sequence (SEQ ID NO: 517) of a native sequence PRO9742 cDNA, wherein SEQ ID NO: 517 is a clone designated herein as “DNA108684-2761”.

FIG. 518 shows the amino acid sequence (SEQ ID NO: 518) derived from the coding sequence of SEQ ID NO: 517 shown in FIG. 517.

FIG. 519 shows a nucleotide sequence (SEQ ID NO: 519) of a native sequence PRO7179 cDNA, wherein SEQ ID NO: 519 is a clone designated herein as “DNA108701-2749”.

FIG. 520 shows the amino acid sequence (SEQ ID NO: 520) derived from the coding sequence of SEQ ID NO: 519 shown in FIG. 519.

FIG. 521 shows a nucleotide sequence (SEQ ID NO: 521) of a native sequence PRO6239 cDNA, wherein SEQ ID NO: 521 is a clone designated herein as “DNA108720-2717”.

FIG. 522 shows the amino acid sequence (SEQ ID NO: 522) derived from the coding sequence of SEQ ID NO: 521 shown in FIG. 521.

FIG. 523 shows a nucleotide sequence (SEQ ID NO: 523) of a native sequence PRO6493 cDNA, wherein SEQ ID NO: 523 is a clone designated herein as “DNA108726-2729”.

FIG. 524 shows the amino acid sequence (SEQ ID NO: 524) derived from the coding sequence of SEQ ID NO: 523 shown in FIG. 523.

FIGS. 525A-525B show a nucleotide sequence (SEQ ID NO: 525) of a native sequence PRO9741 cDNA, wherein SEQ ID NO: 525 is a clone designated herein as “DNA108728-2760”.

FIG. 526 shows the amino acid sequence (SEQ ID NO: 526) derived from the coding sequence of SEQ ID NO: 525 shown in FIGS. 525A-525B.

FIG. 527 shows a nucleotide sequence (SEQ ID NO: 527) of a native sequence PRO9822 cDNA, wherein SEQ ID NO: 527 is a clone designated herein as “DNA108738-2767”.

FIG. 528 shows the amino acid sequence (SEQ ID NO: 528) derived from the coding sequence of SEQ ID NO: 527 shown in FIG. 527.

FIG. 529 shows a nucleotide sequence (SEQ ID NO: 529) of a native sequence PRO6244 cDNA, wherein SEQ ID NO: 529 is a clone designated herein as “DNA108743-2722”.

FIG. 530 shows the amino acid sequence (SEQ ID NO: 530) derived from the coding sequence of SEQ ID NO: 529 shown in FIG. 529.

FIG. 531 shows a nucleotide sequence (SEQ ID NO: 531) of a native sequence PRO9740 cDNA, wherein SEQ ID NO: 531 is a clone designated herein as “DNA108758-2759”.

FIG. 532 shows the amino acid sequence (SEQ ID NO: 532) derived from the coding sequence of SEQ ID NO: 531 shown in FIG. 531.

FIG. 533 shows a nucleotide sequence (SEQ ID NO: 533) of a native sequence PRO9739 cDNA, wherein SEQ ID NO: 533 is a clone designated herein as “DNA108765-2758”.

FIG. 534 shows the amino acid sequence (SEQ ID NO: 534) derived from the coding sequence of SEQ ID NO: 533 shown in FIG. 533.

FIG. 535 shows a nucleotide sequence (SEQ ID NO: 535) of a native sequence PRO7177 cDNA, wherein SEQ ID NO: 535 is a clone designated herein as “DNA108783-2747”.

FIG. 536 shows the amino acid sequence (SEQ ID NO: 536) derived from the coding sequence of SEQ ID NO: 535 shown in FIG. 535.

FIG. 537 shows a nucleotide sequence (SEQ ID NO: 537) of a native sequence PRO7178 cDNA, wherein SEQ ID NO: 537 is a clone designated herein as “DNA108789-2748”.

FIG. 538 shows the amino acid sequence (SEQ ID NO: 538) derived from the coding sequence of SEQ ID NO: 537 shown in FIG. 537.

FIG. 539 shows a nucleotide sequence (SEQ ID NO: 539) of a native sequence PRO6246 cDNA, wherein SEQ ID NO: 539 is a clone designated herein as “DNA108806-2724”.

FIG. 540 shows the amino acid sequence (SEQ ID NO: 540) derived from the coding sequence of SEQ ID NO: 539 shown in FIG. 539.

FIG. 541 shows a nucleotide sequence (SEQ ID NO: 541) of a native sequence PRO6241 cDNA, wherein SEQ ID NO: 541 is a clone designated herein as “DNA108936-2719”.

FIG. 542 shows the amino acid sequence (SEQ ID NO: 542) derived from the coding sequence of SEQ ID NO: 541 shown in FIG. 541.

FIG. 543 shows a nucleotide sequence (SEQ ID NO: 543) of a native sequence PRO9835 cDNA, wherein SEQ ID NO: 543 is a clone designated herein as “DNA1 19510-2771”.

FIG. 544 shows the amino acid sequence (SEQ ID NO: 544) derived from the coding sequence of SEQ ID NO: 543 shown in FIG. 543.

FIG. 545 shows a nucleotide sequence (SEQ ID NO: 545) of a native sequence PRO9857 cDNA, wherein SEQ ID NO: 545 is a clone designated herein as “DNA119517-2778”.

FIG. 546 shows the amino acid sequence (SEQ ID NO: 546) derived from the coding sequence of SEQ ID NO: 545 shown in FIG. 545.

FIG. 547 shows a nucleotide sequence (SEQ ID NO: 547) of a native sequence PRO7436 cDNA, wherein SEQ ID NO: 547 is a clone designated herein as “DNA119535-2756”.

FIG. 548 shows the amino acid sequence (SEQ ID NO: 548) derived from the coding sequence of SEQ ID NO: 547 shown in FIG. 547.

FIG. 549 shows a nucleotide sequence (SEQ ID NO: 549) of a native sequence PRO9856 cDNA, wherein SEQ ID NO: 549 is a clone designated herein as “DNA119537-2777”.

FIG. 550 shows the amino acid sequence (SEQ ID NO: 550) derived from the coding sequence of SEQ ID NO: 549 shown in FIG. 549.

FIG. 551 shows a nucleotide sequence (SEQ ID NO: 551) of a native sequence PRO19605 cDNA, wherein SEQ ID NO: 551 is a clone designated herein as “DNA119714-2851”.

FIG. 552 shows the amino acid sequence (SEQ ID NO: 552) derived from the coding sequence of SEQ ID NO: 551 shown in FIG. 551.

FIG. 553 shows a nucleotide sequence (SEQ ID NO: 553) of a native sequence PRO9859 cDNA, wherein SEQ ID NO: 553 is a clone designated herein as “DNA125170-2780”.

FIG. 554 shows the amino acid sequence (SEQ ID NO: 554) derived from the coding sequence of SEQ ID NO: 553 shown in FIG. 553.

FIG. 555 shows a nucleotide sequence (SEQ ID NO: 555) of a native sequence PRO12970 cDNA, wherein SEQ ID NO: 555 is a clone designated herein as “DNA129594-2841”.

FIG. 556 shows the amino acid sequence (SEQ ID NO: 556) derived from the coding sequence of SEQ ID NO: 555 shown in FIG. 555.

FIG. 557 shows a nucleotide sequence (SEQ ID NO: 557) of a native sequence PRO19626 cDNA, wherein SEQ ID NO: 557 is a clone designated herein as “DNA129793-2857”.

FIG. 558 shows the amino acid sequence (SEQ ID NO: 558) derived from the coding sequence of SEQ ID NO: 557 shown in FIG. 557.

FIG. 559 shows a nucleotide sequence (SEQ ID NO: 559) of a native sequence PRO9833 cDNA, wherein SEQ ID NO: 559 is a clone designated herein as “DNA130809-2769”.

FIG. 560 shows the amino acid sequence (SEQ ID NO: 560) derived from the coding sequence of SEQ ID NO: 559 shown in FIG. 559.

FIG. 561 shows a nucleotide sequence (SEQ ID NO: 561) of a native sequence PRO19670 cDNA, wherein SEQ ID NO: 561 is a clone designated herein as “DNA131639-2874”.

FIG. 562 shows the amino acid sequence (SEQ ID NO: 562) derived from the coding sequence of SEQ ID NO: 561 shown in FIG. 561.

FIG. 563 shows a nucleotide sequence (SEQ ID NO: 563) of a native sequence PRO19624 cDNA, wherein SEQ ID NO: 563 is a clone designated herein as “DNA131649-2855”.

FIG. 564 shows the amino acid sequence (SEQ ID NO: 564) derived from the coding sequence of SEQ ID NO: 563 shown in FIG. 563.

FIG. 565 shows a nucleotide sequence (SEQ ID NO: 565) of a native sequence PRO19680 cDNA, wherein SEQ ID NO: 565 is a clone designated herein as “DNA131652-2876”.

FIG. 566 shows the amino acid sequence (SEQ ID NO: 566) derived from the coding sequence of SEQ ID NO: 565 shown in FIG. 565.

FIG. 567 shows a nucleotide sequence (SEQ ID NO: 567) of a native sequence PRO 19675 cDNA, wherein SEQ ID NO: 567 is a clone designated herein as “DNA131658-2875”.

FIG. 568 shows the amino acid sequence (SEQ ID NO: 568) derived from the coding sequence of SEQ ID NO: 567 shown in FIG. 567.

FIG. 569 shows a nucleotide sequence (SEQ ID NO: 569) of a native sequence PRO9834 cDNA, wherein SEQ ID NO: 569 is a clone designated herein as “DNA132162-2770”.

FIG. 570 shows the amino acid sequence (SEQ ID NO: 570) derived from the coding sequence of SEQID NO: 569 shown in FIG. 569.

FIG. 571 shows a nucleotide sequence (SEQ ID NO: 571) of a native sequence PRO9744 cDNA, wherein SEQ ID NO: 571 is a clone designated herein as “DNA136110-2763”.

FIG. 572 shows the amino acid sequence (SEQ ID NO: 572) derived from the coding sequence of SEQ ID NO: 571 shown in FIG. 571.

FIG. 573 shows a nucleotide sequence (SEQ ID NO: 573) of a native sequence PRO19644 cDNA, wherein SEQ ID NO: 573 is a clone designated herein as “DNA139592-2866”.

FIG. 574 shows the amino acid sequence (SEQ ID NO: 574) derived from the coding sequence of SEQ ID NO: 573 shown in FIG. 573.

FIG. 575 shows a nucleotide sequence (SEQ ID NO: 575) of a native sequence PRO19625 cDNA, wherein SEQ ID NO: 575 is a clone designated herein as “DNA139608-2856”.

FIG. 576 shows the amino acid sequence (SEQ ID NO: 576) derived from the coding sequence of SEQ ID NO: 575 shown in FIG. 575.

FIG. 577 shows a nucleotide sequence (SEQ ID NO: 577) of a native sequence PRO19597 cDNA, wherein SEQ ID NO: 577 is a clone designated herein as “DNA143292-2848”.

FIG. 578 shows the amino acid sequence (SEQ ID NO: 578) derived from the coding sequence of SEQ ID NO: 577 shown in FIG. 577.

FIG. 579 shows a nucleotide sequence (SEQ ID NO: 579) of a native sequence PRO16090 cDNA, wherein SEQ ID NO: 579 is a clone designated herein as “DNA144844-2843”.

FIG. 580 shows the amino acid sequence (SEQ ID NO: 580) derived from the coding sequence of SEQ ID NO: 579 shown in FIG. 579.

FIG. 581 shows a nucleotide sequence (SEQ ID NO: 581) of a native sequence PRO19576 cDNA, wherein SEQ ID NO: 581 is a clone designated herein as “DNA144857-2845”.

FIG. 582 shows the amino acid sequence (SEQ ID NO: 582) derived from the coding sequence of SEQ ID NO: 581 shown in FIG. 581.

FIG. 583 shows a nucleotide sequence (SEQ ID NO: 583) of a native sequence PRO19646 cDNA, wherein SEQ ID NO: 583 is a clone designated herein as “DNA145841-2868”.

FIG. 584 shows the amino acid sequence (SEQ ID NO: 584) derived from the coding sequence of SEQ ID NO: 583 shown in FIG. 583.

FIG. 585 shows a nucleotide sequence (SEQ ID NO: 585) of a native sequence PRO19814 cDNA, wherein SEQ ID NO: 585 is a clone designated herein as “DNA148004-2882”.

FIG. 586 shows the amino acid sequence (SEQ ID NO: 586) derived from the coding sequence of SEQ ID NO: 585 shown in FIG. 585.

FIG. 587 shows a nucleotide sequence (SEQ ID NO: 587) of a native sequence PRO19669 cDNA, wherein SEQ ID NO: 587 is a clone designated herein as “DNA149893-2873”.

FIG. 588 shows the amino acid sequence (SEQ ID NO: 588) derived from the coding sequence of SEQ ID NO: 587 shown in FIG. 587.

FIG. 589 shows a nucleotide sequence (SEQ ID NO: 589) of a native sequence PRO19818 cDNA, wherein SEQ ID NO: 589 is a clone designated herein as “DNA149930-2884”.

FIG. 590 shows the amino acid sequence (SEQ ID NO: 590) derived from the coding sequence of SEQ ID NO: 589 shown in FIG. 589.

FIG. 591 shows a nucleotide sequence (SEQ ID NO: 591) of a native sequence PRO20088 cDNA, wherein SEQ ID NO: 591 is a clone designated herein as “DNA150157-2898”.

FIG. 592 shows the amino acid sequence (SEQ ID NO: 592) derived from the coding sequence of SEQ ID NO: 591 shown in FIG. 591.

FIG. 593 shows a nucleotide sequence (SEQ ID NO: 593) of a native sequence PRO16089 cDNA, wherein SEQ ID NO: 593 is a clone designated herein as “DNA150163-2842”.

FIG. 594 shows the amino acid sequence (SEQ ID NO: 594) derived from the coding sequence of SEQ ID NO: 593 shown in FIG. 593.

FIG. 595 shows a nucleotide sequence (SEQ ID NO: 595) of a native sequence PRO20025 cDNA, wherein SEQ ID NO: 595 is a clone designated herein as “DNA153579-2894”.

FIG. 596 shows the amino acid sequence (SEQ ID NO: 596) derived from the coding sequence of SEQ ID NO: 595 shown in FIG. 595.

FIG. 597 shows a nucleotide sequence (SEQ ID NO: 597) of a native sequence PRO20040 cDNA, wherein SEQ ID NO: 597 is a clone designated herein as “DNA164625-2890”.

FIG. 598 shows the amino acid sequence (SEQ ID NO: 598) derived from the coding sequence of SEQ ID NO: 597 shown in FIG. 597.

FIG. 599 shows a nucleotide sequence (SEQ ID NO: 599) of a native sequence PRO791 cDNA, wherein SEQ ID NO: 599 is a clone designated herein as “DNA57838-1337”.

FIG. 600 shows the amino acid sequence (SEQ ID NO: 600) derived from the coding sequence of SEQ ID NO: 599 shown in FIG. 599.

FIG. 601 shows a nucleotide sequence (SEQ ID NO: 601) of a native sequence PRO 1131 cDNA, wherein SEQ ID NO: 601 is a clone designated herein as “DNA59777-1480”.

FIG. 602 shows the amino acid sequence (SEQ ID NO: 602) derived from the coding sequence of SEQ ID NO: 601 shown in FIG. 601.

FIG. 603 shows a nucleotide sequence (SEQ ID NO: 603) of a native sequence PRO1343 cDNA, wherein SEQ ID NO: 603 is a clone designated herein as “DNA66675-1587”.

FIG. 604 shows the amino acid sequence (SEQ ID NO: 604) derived from the coding sequence of SEQ ID NO: 603 shown in FIG. 603.

FIG. 605 shows a nucleotide sequence (SEQ ID NO: 605) of a native sequence PRO1760 cDNA, wherein SEQ ID NO: 605 is a clone designated herein as “DNA76532-1702”.

FIG. 606 shows the amino acid sequence (SEQ ID NO: 606) derived from the coding sequence of SEQ ID NO: 605 shown in FIG. 605.

FIG. 607 shows a nucleotide sequence (SEQ ID NO: 607) of a native sequence PRO6029 cDNA, wherein SEQ ID NO: 607 is a clone designated herein as “DNA105849-2704”.

FIG. 608 shows the amino acid sequence (SEQ ID NO: 608) derived from the coding sequence of SEQ ID NO: 607 shown in FIG. 607.

FIG. 609 shows a nucleotide sequence (SEQ ID NO: 609) of a native sequence PRO1801 cDNA, wherein SEQ ID NO: 609 is a clone designated herein as “DNA83500-2506”.

FIG. 610 shows the amino acid sequence (SEQ ID NO: 610) derived from the coding sequence of SEQ ID NO: 609 shown in FIG. 609.

### DETAILED DESCRIPTION OF THE PREFERRED EMBODIMENTS

I. Definitions

The terms “PRO polypeptide” and “PRO” as used herein and when immediately followed by a numerical designation refer to various polypeptides, wherein the complete designation (i.e., PRO/number) refers to specific polypeptide sequences as described herein. The terms “PRO/number polypeptide” and “PRO/number” wherein the term “number” is provided as an actual numerical designation as used herein encompass native sequence polypeptides and polypeptide variants (which are further defined herein). The PRO polypeptides described herein may be isolated from a variety of sources, such as from human tissue types or from another source, or prepared by recombinant or synthetic methods. The term “PRO polypeptide” refers to each individual PRO/number polypeptide disclosed herein. All disclosures in this specification which refer to the “PRO polypeptide” refer to each of the polypeptides individually as well as jointly. For example, descriptions of the preparation of, purification of, derivation of, formation of antibodies to or against, administration of, compositions containing, treatment of a disease with, etc., pertain to each polypeptide of the invention individually. The term “PRO polypeptide” also includes variants of the PRO/number polypeptides disclosed herein.

A “native sequence PRO polypeptide” comprises a polypeptide having the same amino acid sequence as the corresponding PRO polypeptide derived from nature. Such native sequence PRO polypeptides can be isolated from nature or can be produced by recombinant or synthetic means. The term “native sequence PRO polypeptide” specifically encompasses naturally-occurring truncated or secreted forms of the specific PRO polypeptide (e.g., an extracellular domain sequence), naturally-occurring variant forms (e.g., alternatively spliced forms) and naturally-occurring allelic variants of the polypeptide. In various embodiments of the invention, the native sequence PRO polypeptides disclosed herein are mature or full-length native sequence polypeptides comprising the full-length amino acids sequences shown in the accompanying figures. Start and stop codons are shown in bold font and underlined in the figures. However, while the PRO polypeptide disclosed in the accompanying figures are shown to begin with methionine residues designated herein as amino acid position 1 in the figures, it is conceivable and possible that other methionine residues located either upstream or downstream from the amino acid position 1 in the figures may be employed as the starting amino acid residue for the PRO polypeptides.

The PRO polypeptide “extracellular domain” or “ECD” refers to a form of the PRO polypeptide which is essentially free of the transmembrane and cytoplasmic domains. Ordinarily, a PRO polypeptide ECD will have less than 1% of such transmembrane and/or cytoplasmic domains and preferably, will have less than 0.5% of such domains. It will be understood that any transmembrane domains identified for the PRO polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified herein. Optionally, therefore, an extracellular domain of a PRO polypeptide may contain from about 5 or fewer amino acids on either side of the transmembrane domain/extracellular domain boundary as identified in the Examples or specification and such polypeptides, with or without the associated signal peptide, and nucleic acid encoding them, are contemplated by the present invention.

The approximate location of the “signal peptides” of the various PRO polypeptides disclosed herein are shown in the present specification and/or the accompanying figures. It is noted, however, that the C-terminal boundary of a signal peptide may vary, but most likely by no more than about 5 amino acids on either side of the signal peptide C-terminal boundary as initially identified herein, wherein the C-terminal boundary of the signal peptide may be identified pursuant to criteria routinely employed in the art for identifying that type of amino acid sequence element (e.g., Nielsen et al., Prot. Eng. 10:1-6 (1997) and von Heinje et al., Nucl. Acids. Res. 14:4683-4690 (1986)). Moreover, it is also recognized that, in some cases, cleavage of a signal sequence from a secreted polypeptide is not entirely uniform, resulting in more than one secreted species. These mature polypeptides, where the signal peptide is cleaved within no more than about 5 amino acids on either side of the C-terminal boundary of the signal peptide as identified herein, and the polynucleotides encoding them, are contemplated by the present invention.

“PRO polypeptide variant” means an active PRO polypeptide as defined above or below having at least about 80% amino acid sequence identity with a full-length native sequence PRO polypeptide sequence as disclosed herein, a PRO polypeptide sequence lacking the signal peptide as disclosed herein, an extracellular domain of a PRO polypeptide, with or without the signal peptide, as disclosed herein or any other fragment of a full-length PRO polypeptide sequence as disclosed herein. Such PRO polypeptide variants include, for instance, PRO polypeptides wherein one or more amino acid residues are added, or deleted, at the N- or C-terminus of the full-length native amino acid sequence. Ordinarily, a PRO polypeptide variant will have at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a full-length native sequence PRO polypeptide sequence as disclosed herein, a PRO polypeptide sequence lacking the signal peptide as disclosed herein, an extracellular domain of a PRO polypeptide, with or without the signal peptide, as disclosed herein or any other specifically defined fragment of a full-length PRO polypeptide sequence as disclosed herein. Ordinarily, PRO variant polypeptides are at least about 10 amino acids in length, alternatively at least about 20 amino acids in length, alternatively at least about 30 amino acids in length, alternatively at least about 40 amino acids in length, alternatively at least about 50 amino acids in length, alternatively at least about 60 amino acids in length, alternatively at least about 70 amino acids in length, alternatively at least about 80 amino acids in length, alternatively at least about 90 amino acids in length, alternatively at least about 100 amino acids in length, alternatively at least about 150 amino acids in length, alternatively at least about 200 amino acids in length, alternatively at least about 300 amino acids in length, or more.

“Percent (%) amino acid sequence identity” with respect to the PRO polypeptide sequences identified herein is defined as the percentage of amino acid residues in a candidate sequence that are identical with the amino acid residues in the specific PRO polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations using this method, Tables 2 and 3 demonstrate how to calculate the % amino acid sequence identity of the amino acid sequence designated “Comparison Protein” to the amino acid sequence designated “PRO”, wherein “PRO” represents the amino acid sequence of a hypothetical PRO polypeptide of interest, “Comparison Protein” represents the amino acid sequence of a polypeptide against which the “PRO” polypeptide of interest is being compared, and “X, “Y” and “Z” each represent different hypothetical amino acid residues.

Unless specifically stated otherwise, all % amino acid sequence identity values used herein are obtained as described in the immediately preceding paragraph using the ALIGN-2 computer program. However, % amino acid sequence identity values may also be obtained as described below by using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % amino acid sequence identity value is determined by dividing (a) the number of matching identical amino acid residues between the amino acid sequence of the PRO polypeptide of interest having a sequence derived from the native PRO polypeptide and the comparison amino acid sequence of interest (i.e., the sequence against which the PRO polypeptide of interest is being compared which may be a PRO variant polypeptide) as determined by WU-BLAST-2 by (b) the total number of amino acid residues of the PRO polypeptide of interest. For example, in the statement “a polypeptide comprising an the amino acid sequence A which has or having at least 80% amino acid sequence identity to the amino acid sequence B”, the amino acid sequence A is the comparison amino acid sequence of interest and the amino acid sequence B is the amino acid sequence of the PRO polypeptide of interest.

Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“PRO variant polynucleotide” or “PRO variant nucleic acid sequence” means a nucleic acid molecule which encodes an active PRO polypeptide as defined below and which has at least about 80% nucleic acid sequence identity with a nucleotide acid sequence encoding a full-length native sequence PRO polypeptide sequence as disclosed herein, a full-length native sequence PRO polypeptide sequence lacking the signal peptide as disclosed herein, an extracellular domain of a PRO polypeptide, with or without the signal peptide, as disclosed herein or any other fragment of a full-length PRO polypeptide sequence as disclosed herein. Ordinarily, a PRO variant polynucleotide will have at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity with a nucleic acid sequence encoding a full-length native sequence PRO polypeptide sequence as disclosed herein, a full-length native sequence PRO polypeptide sequence lacking the signal peptide as disclosed herein, an extracellular domain of a PRO polypeptide, with or without the signal sequence, as disclosed herein or any other fragment of a full-length PRO polypeptide sequence as disclosed herein. Variants do not encompass the native nucleotide sequence.

Ordinarily, PRO variant polynucleotides are at least about 30 nucleotides in length, alternatively at least about 60 nucleotides in length, alternatively at least about 90 nucleotides in length, alternatively at least about 120 nucleotides in length, alternatively at least about 150 nucleotides in length, alternatively at least about 180 nucleotides in length, alternatively at least about 210 nucleotides in length, alternatively at least about 240 nucleotides in length, alternatively at least about 270 nucleotides in length, alternatively at least about 300 nucleotides in length, alternatively at least about 450 nucleotides in length, alternatively at least about 600 nucleotides in length, alternatively at least about 900 nucleotides in length, or more.

“Percent (%) nucleic acid sequence identity” with respect to PRO-encoding nucleic acid sequences identified herein is defined as the percentage of nucleotides in a candidate sequence that are identical with the nucleotides in the PRO nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. For purposes herein, however, % nucleic acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 4 and 5, demonstrate how to calculate the % nucleic acid sequence identity of the nucleic acid sequence designated “Comparison DNA” to the nucleic acid sequence designated “PRO-DNA”, wherein “PRO-DNA” represents a hypothetical PRO-encoding nucleic acid sequence of interest, “Comparison DNA” represents the nucleotide sequence of a nucleic acid molecule against which the “PRO-DNA” nucleic acid molecule of interest is being compared, and “N”, “L” and “V” each represent different hypothetical nucleotides.

Unless specifically stated otherwise, all % nucleic acid sequence identity values used herein are obtained as described in the immediately preceding paragraph using the ALIGN-2 computer program. However, % nucleic acid sequence identity values may also be obtained as described below by using the WU-BLAST-2 computer program (Altschul et al., Methods in Enzymology 266:460-480 (1996)).Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % nucleic acid sequence identity value is determined by dividing (a) the number of matching identical nucleotides between the nucleic acid sequence of the PRO polypeptide-encoding nucleic acid molecule of interest having a sequence derived from the native sequence PRO polypeptide-encoding nucleic acid and the comparison nucleic acid molecule of interest (i.e., the sequence against which the PRO polypeptide-encoding nucleic acid molecule of interest is being compared which may be a variant PRO polynucleotide) as determined by WU-BLAST-2 by (b) the total number of nucleotides of the PRO polypeptide-encoding nucleic acid molecule of interest. For example, in the statement “an isolated nucleic acid molecule comprising a nucleic acid sequence A which has or having at least 80% nucleic acid sequence identity to the nucleic acid sequence B”, the nucleic acid sequence A is the comparison nucleic acid molecule of interest and the nucleic acid sequence B is the nucleic acid sequence of the PRO polypeptide-encoding nucleic acid molecule of interest.

Percent nucleic acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

In other embodiments, PRO variant polynucleotides are nucleic acid molecules that encode an active PRO polypeptide and which are capable of hybridizing, preferably under stringent hybridization and wash conditions, to nucleotide sequences encoding a full-length PRO polypeptide as disclosed herein. PRO variant polypeptides may be those that are encoded by a PRO variant polynucleotide.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the PRO polypeptide natural environment will not be present. Ordinarily, however, isolated polypeptide will be prepared by at least one purification step.

An “isolated” PRO polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers, for example, single anti-PRO monoclonal antibodies (including agonist, antagonist, and neutralizing antibodies), anti-PRO antibody compositions with polyepitopic specificity, single chain anti-PRO antibodies, and fragments of anti-PRO antibodies (see below). The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5× SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2× SSC (sodium chloride/sodium citrate) and 50% formamide at 55° C., followed by a high-stringency wash consisting of 0.1× SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5× SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1× SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a PRO polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

“Active” or “activity” for the purposes herein refers to form(s) of a PRO polypeptide which retain a biological and/or an immunological activity of native or naturally-occurring PRO, wherein “biological” activity refers to a biological function (either inhibitory or stimulatory) caused by a native or naturally-occurring PRO other than the ability to induce the production of an antibody against an antigenic epitope possessed by a native or naturally-occurring PRO and an “immunological” activity refers to the ability to induce the production of an antibody against an antigenic epitope possessed by a native or naturally-occurring PRO.

The term “antagonist” is used in the broadest sense, and includes any molecule that partially or fully blocks, inhibits, or neutralizes a biological activity of a native PRO polypeptide disclosed herein. In a similar manner, the term “agonist” is used in the broadest sense and includes any molecule that mimics a biological activity of a native PRO polypeptide disclosed herein. Suitable agonist or antagonist molecules specifically include agonist or antagonist antibodies or antibody fragments, fragments or amino acid sequence variants of native PRO polypeptides, peptides, antisense oligonucleotides, small organic molecules, etc. Methods for identifying agonists or antagonists of a PRO polypeptide may comprise contacting a PRO polypeptide with a candidate agonist or antagonist molecule and measuring a detectable change in one or more biological activities normally associated with the PRO polypeptide.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

The word “label” when used herein refers to a detectable compound or composition which is conjugated directly or indirectly to the antibody so as to generate a “labeled” antibody. The label may be detectable by itself (e.g. radioisotope labels or fluorescent labels) or, in the case of an enzymatic label, may catalyze chemical alteration of a substrate compound or composition which is detectable.

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a PRO polypeptide or antibody thereto) to a mammal. The components of the liposome are commonly arranged in a bilayer formation, similar to the lipid arrangement of biological membranes.

A “small molecule” is defined herein to have a molecular weight below about 500 Daltons.

An “effective amount” of a polypeptide disclosed herein or an agonist or antagonist thereof is an amount sufficient to carry out a specifically stated purpose. An “effective amount” may be determined empirically and in a routine manner, in relation to the stated purpose.

TABLE 1
/*
*
* C—C increased from 12 to 15
* Z is average of EQ
* B is average of ND
* match with stop is _M; stop—stop = 0; J (joker) match = 0
*/

| | | | |
|---|---|---|---|
| #define | _M | −8 | /* value of a match with a stop */ |

| | |
|---|---|
| int | _day[26][26] = { |

/* A B C D E F G H I J K L M N O P Q R S T U V W X Y Z */

| | |
|---|---|
| /* A */ | {2, 0, −2, 0, 0, −4, 1, −1, −1, 0, −1, −2, −1, 0, _M, 1, 0, −2, 1, 1, 0, 0, −6, 0, −3, 0}, |
| /* B */ | {0, 3, −4, 3, 2, −5, 0, 1, −2, 0, 0, −3, −2, 2, _M, −1, 1, 0, 0, 0, 0, −2, −5, 0, −3, 1}, |
| /* C */ | {−2, −4, 15, −5, −5, −4, −3, −3, −2, 0, −5, −6, −5, −4, _M, −3, −5, −4, 0, −2, 0, −2, −8, 0, 0, −5}, |
| /* D */ | {0, 3, −5, 4, 3, −6, 1, 1, −2, 0, 0, −4, −3, 2, _M, −1, 2, −1, 0, 0, 0, −2, −7, 0, −4, 2}, |
| /* E */ | {0, 2, −5, 3, 4, −5, 0, 1, −2, 0, 0, −3, −2, 1, _M, −1, 2, −1, 0, 0, 0, −2, −7, 0, −4, 3}, |
| /* F */ | {−4, −5, −4, −6, −5, 9, −5, −2, 1, 0, −5, 2, 0, −4, _M, −5, −5, −4, −3, −3, 0, −1, 0, 0, 7, −5}, |
| /* G */ | {1, 0, −3, 1, 0, −5, 5, −2, −3, 0, −2, −4, −3, 0, _M, −1, −1, −3, 1, 0, 0, −1, −7, 0, −5, 0}, |
| /* H */ | {−1, 1, −3, 1, 1, −2, −2, 6, −2, 0, 0, −2, −2, 2, _M, 0, 3, 2, −1, −1, 0, −2, −3, 0, 0, 2}, |
| /* I */ | {−1, −2, −2, −2, −2, 1, −3, −2, 5, 0, −2, 2, 2, −2, _M, −2, −2, −2, −1, 0, 0, 4, −5, 0, −1, −2}, |
| /* J */ | {0, 0, 0, 0, 0, 0, 0, 0, 0, 0, 0, 0, 0, 0, _M, 0, 0, 0, 0, 0, 0, 0, 0, 0, 0, 0}, |
| /* K */ | {−1, 0, −5, 0, 0, −5, −2, 0, −2, 0, 5, −3, 0, 1, _M, −1, 1, 3, 0, 0, 0, −2, −3, 0, −4, 0}, |
| /* L */ | {−2, −3, −6, −4, −3, 2, −4, −2, 2, 0, −3, 6, 4, −3, _M, −3, −2, −3, −3 , −1, 0, 2, −2, 0, −1, −2} |
| /* M */ | {−1, −2, −5, −3, −2, 0, −3, −2, 2, 0, 0, 4, 6, −2, _M, −2, −1, 0, −2, −1, 0, 2, −4, 0, −2, −1}, |
| /* N */ | {0, 2, −4, 2, 1, −4, 0, 2, −2, 0, 1, −3, −2, 2, _M, −1, 1, 0, 1, 0, 0, −2, −4, 0, −2, 1}, |
| /* O */ | {_M,_M,_M,_M,_M,_M,_M,_M,_M,_M,_M,_M,_M,_M, 0,_M,_M,_M,_M,_M,_M,_M,_M,_M,_M,_M,}, |
| /* P */ | {1, −1, −3, −1, −1, −5, −1, 0, −2, 0, −1, −3, −2, −1,_M, 6, 0, 0, 1, 0, 0, −1, −6, 0, −5, 0}, |
| /* Q */ | {0, 1, −5, 2, 2, −5, −1, 3, −2, 0, 1, −2, −1, 1, _M, 0, 4, 1, −1, −1, 0, −2, −5, 0, −4, 3}, |
| /* R */ | {−2, 0, −4, −1, −1, −4, −3, 2, −2, 0, 3, −3, 0, 0, _M, 0, 1, 6, 0, −1, 0, −2, 2, 0, −4, 0}, |
| /* S */ | {1, 0, 0, 0, 0, −3, 1, −1, −1, 0, 0, −3, −2, 1, _M, 1, −1, 0, 2, 1, 0, −1, −2, 0, −3, 0}, |
| /* T */ | {1, 0, −2, 0, 0, −3, 0, −1, 0, 0, 0, −1, −1, 0, _M, 0, −1, −1, 1, 3, 0, 0, −5, 0, −3, 0}, |
| /* U */ | {0, 0, 0, 0, 0, 0, 0, 0, 0, 0, 0, 0, 0, 0,_M, 0, 0, 0, 0, 0, 0, 0, 0, 0, 0, 0}, |
| /* V */ | {0, −2, −2, −2, −2, −1, −1, −2, 4, 0, −2, 2, 2, −2,_M, −1, −2, −2, −1, 0, 0, 4, −6, 0, −2, −2}, |
| /* W */ | {−6, −5, −8, −7, −7, 0, −7, −3, −5, 0, −3, −2, −4, −4,_M, −6, −5, 2, −2, −5, 0, −6, 17, 0, 0, −6}, |
| /* X */ | {0, 0, 0, 0, 0, 0, 0, 0, 0, 0, 0, 0, 0, 0, _M, 0, 0, 0, 0, 0, 0, 0, 0, 0, 0, 0}, |
| /* Y */ | {−3, −3, 0, −4, −4, 7, −5, 0, −1, 0, −4, −1, −2, −2, _M, −5, −4, −4, −3, −3, 0, −2, 0, 0, 10, −4}, |
| /* Z */ | {0, 1, −5, 2, 3, −5, 0, 2, −2, 0, 0, −2, −1, 1,_M, 0, 3, 0, 0, 0, 0, −2, −6, 0, −4, 4}, |
| }; | |

/*
*/
#include <stdio.h>
#include <ctype.h>

| | | |
|---|---|---|
| #define MAXJMP | 16 | /* max jumps in a diag */ |
| #define MAXGAP | 24 | /* don't continue to penalize gaps larger than this */ |
| #define JMPS | 1024 | /* max jmps in an path */ |
| #define MX | 4 | /* save if there's at least MX-1 bases since last jmp */ |
| #define DMAT | 3 | /* value of matching bases */ |
| #define DMIS | 0 | /* penalty for mismatched bases */ |
| #define DINS0 | 8 | /* penalty for a gap */ |
| #define DINS1 | 1 | /* penalty per base */ |
| #define PINS0 | 8 | /* penalty for a gap */ |
| #define PINS1 | 4 | /* penalty per residue */ |
| struct jmp { | | |

| | | |
|---|---|---|
| short | n[MAXJMP]; | /* size of jmp (neg for dely) */ |
| unsigned short | x[MAXJMP]; | /* base no. of jmp in seq x */ |

/* limits seq to 2\hat{ }16 −1 */

};
struct diag {

| | | |
|---|---|---|
| int | score; | /* score at last jmp */ |
| long | offset; | /* offset of prev block */ |
| short | ijmp; | /* current jmp index */ |
| struct jmp | jp; | /* list of jmps */ |

};
struct path {

| | | |
|---|---|---|
| int | spc; | /* number of leading spaces */ |
| short | n[JMPS]; | /* size of jmp (gap) */ |
| int | x[JMPS]; | /* loc of jmp (last elem before gap) */ |

};

| | | |
|---|---|---|
| char | *ofile; | /* output file name */ |
| char | *namex[2]; | /* seq names: getseqs() */ |
| char | *prog; | /* prog name for err msgs */ |
| char | *seqx[2]; | /* seqs: getseqs() */ |
| int | dmax; | /* best diag: nw() */ |
| int | dmax0; | /* final diag */ |
| int | dna; | /* set if dna: main() */ |
| int | endgaps; | /* set if penalizing end gaps */ |
| int | gapx, gapy; | /* total gaps in seqs */ |
| int | len0, len1; | /* seq lens */ |
| int | ngapx, ngapy; | /* total size of gaps */ |
| int | smax; | /* max score: nw() */ |
| int | *xbm; | /* bitmap for matching */ |
| long | offset; | /* current offset in jmp file */ |
| struct diag | *dx; | /* holds diagonals */ |
| struct path | pp[2]; | /* holds path for seqs */ |

| | |
|---|---|
| char | *calloc(), *malloc(), *index(), *strcpy(); |
| char | *getseq(), *g_calloc(); |

/* Needleman-Wunsch alignment program
*
* usage: progs file1 file2
* where file1 and file2 are two dna or two protein sequences.
* The sequences can be in upper- or lower-case an may contain ambiguity
* Any lines beginning with ‘;’, ‘>’ or ‘<’ are ignored
* Max file length is 65535 (limited by unsigned short x in the jmp struct)
* A sequence with ⅓ or more of its elements ACGTU is assumed to be DNA
* Output is in the file “align.out”
*
* The program may create a tmp file in /tmp to hold info about traceback.
* Original version developed under BSD 4.3 on a vax 8650
*/
#include “nw.h”
#include “day.h”

| | |
|---|---|
| static | _dbval[26] = { |
| | 1,14,2,13,0,0,4,11,0,0,12,0,3,15,0,0,0,5,6,8,8,7,9,0,10,0 |
| }; | |
| static | _pbval[26] = { |
| | 1, 2\|(1<<(‘D’-‘A’))\|(1<<(‘N’-‘A’)), 4, 8, 16, 32, 64, |
| | 128, 256, 0×FFFFFFF, 1<<10, 1<<11, 1<<12, 1<<13, 1<<14, |
| | 1<<15, 1<<16, 1< <17, 1<<18, 1<<19, 1<<20, 1<<21, 1<<22, |
| | 1<<23, 1<<24, 1<<25\|(1<<(‘E’-‘A’))\|(1<<(‘Q’-‘A’)) |

};

| | |
|---|---|
| main(ac, av) | main |

| | |
|---|---|
| int | ac; |
| char | *av[]; |

{

prog = av[0];
if(ac != 3) {

fprintf(stderr, “usage: %s file1 file2\n”, prog);
fprintf(stderr, “where file1 and file2 are two dna or two protein sequences.\n”);
fprintf(stderr, “The sequences can be in upper- or lower-case\n”);
fprintf(stderr, “Any lines beginning with ‘;’ or ‘<’ are ignored\n”);
fprintf(stderr, “Output is in the file \“align.out\”\n”);
exit(1);

}
namex[0] = av[1];
namex[1] = av[2];
seqx[0] = getseq(namex[0], &len0);
seqx[1] = getseq(namex[1], &len1);
xbm = (dna)? _dbval : _pbval;

| | |
|---|---|
| endgaps = 0; | /* 1 to penalize endgaps */ |
| ofile = “align.out”; | /* output file */ |

| | |
|---|---|
| nw(); | /* fill in the matrix, get the possible jmps */ |
| readjmps(); | /* get the actual jmps */ |
| print(); | /* print stats, alignment */ |
| cleanup(0); | /* unlink any tmp files */ |

}
/* do the alignment, return best score: main()
* dna: values in Fitch and Smith, PNAS, 80, 1382-1386, 1983
* pro: PAM 250 values
* When scores are equal, we prefer mismatches to any gap, prefer
* a new gap to extending an ongoing gap, and prefer a gap in seqx
* to a gap in seq y.
*/

| | |
|---|---|
| nw() | nw |
| { | |

| | | |
|---|---|---|
| char | *px, *py; | /* seqs and ptrs */ |
| int | *ndely, *dely; | /* keep track of dely */ |
| int | ndelx, delx; | /* keep track of delx */ |
| int | *tmp; | /* for swapping row0, row1 */ |
| int | mis; | /* score for each type */ |
| int | ins0, ins1; | /* insertion penalties */ |
| register | id; | /* diagonal index */ |
| register | ij; | /* jmp index */ |
| register | *col0, *col1; | /* score for curr, last row */ |
| register | xx, yy; | /* index into seqs */ |

dx = (struct diag *)g_calloc(“to get diags”, len0+len1+1, sizeof(struct diag));
ndely = (int *)g_calloc(“to get ndely”, len1+1, sizeof(int));
dely = (int *)g_calloc(“to get dely”, len1+1, sizeof(int));
col0 = (int *)g_calloc(“to get col0”, len1+1, sizeof(int));
col1 = (int *)g_calloc(“to get col1”, len1+1, sizeof(int));
ins0 = (dna)? DINS0 : PINS0;
ins1 = (dna)? DINS1 : PlNS1;
smax = −10000;
if (endgaps) {

for (col0[0] = dely[0] = −ins0, yy = 1; yy <= len1; yy++) {

col0[yy] = dely[yy] = col0[yy−1] − ins1;
ndely[yy] = yy;

| | |
|---|---|
| } | |
| col0[0] = 0; | /* Waterman Bull Math Biol 84 */ |

}
else

for (yy = 1; yy <= len1; yy++)

dely[yy] = −ins0;

/* fill in match matrix
*/
for (px = seqx[0], xx = 1; xx <= len0; px++, xx++) {

/* initialize first entry in col
*/
if (endgaps) {

if (xx == 1)

col1[0] = delx = −(ins0+ins1);

else

col1[0] = delx = col0[0]−ins1;

ndelx = xx;

}
else {

col1[0] = 0;
delx = −ins0;
ndelx = 0;

}

...nw

for (py = seqx[1], yy = 1; yy <= len1; py++, yy++) {

mis = col0[yy−1];
if (dna)

mis += (xbm[*px−‘A’]&xbm[*py−‘A’])? DMAT : DMIS;

else

mis += _day[*px−‘A’][*py−‘A’];

/* update penalty for del in x seq;
* favor new del over ongong del
* ignore MAXGAP if weighting endgaps
*/
if (endgaps || ndely[yy] < MAXGAP) {

if (col0[yy] − ins0 >= dely[yy]) {

dely[yy] = col0[yy] − (ins0+ins1);
ndely[yy] = 1;

} else {

dely[yy] −= ins1;
ndely[yy]++;

}

} else {

if (col0[yy] − (ins0+ins1) >= dely[yy]) {

dely[yy] = col0[yy] − (ins0+ins1);
ndely[yy] = 1;

} else

ndely[yy]++;

}
/* update penalty for del in y seq;
* favor new del over ongong del
*/
if (endgaps || ndelx < MAXGAP) {

if(col1[yy−1] − ins0 >= delx) {

delx = col1[yy−1] − (ins0+ins1);
ndelx = 1;

} else {

delx −= ins1;
ndelx++;

}

} else {

if (col1[yy−1] − (ins0+ins1) >= delx) {

delx = col1[yy−1] − (ins0+ins1);
ndelx = 1;

} else

ndelx++;

}
/* pick the maximum score; we're favoring
* mis over any del and delx over dely
*/

...nw

id = xx − yy + len1 − 1;
if (mis >= delx && mis >= dely[yy])

col1[yy] = mis;

else if (delx >= dely[yy]) {

col1[yy] = delx;
ij = dx[id].ijmp;
if (dx[id].jp.n[0] && (!dna || (ndelx >= MAXJMP
&& xx > dx[id].jp.x[ij]+MX) || mis > dx[id].score+DINS0)) {

dx[id].ijmp++;
if (++ij >= MAXJMP) {

writejmps(id);
ij = dx[id].ijmp = 0;
dx[id].offset = offset;
offset += sizeof(struct jmp) + sizeof(offset);

}

}
dx[id].jp.n[ij] = ndelx;
dx[id].jp.x[ij] = xx;
dx[id].score = delx;

}
else {

col1[yy] = dely[yy];
ij = dx[id].ijmp;

if (dx[id].jp.n[0] && (!dna || (ndely[yy] >= MAXJMP

&& xx > dx[id].jp.x[ij]+MX) || mis > dx[id].score+DINS0)) {

dx[id].ijmp++;
if (++ij >= MAXJMP) {

writejmps(id);
ij = dx[id].ijmp = 0;
dx[id].offset = offset;
offset += sizeof(struct jmp) + sizeof(offset);

}

}
dx[id].jp.n[ij] =− ndely[yy];
dx[id].jp.x[ij] = xx;
dx[id].score = dely[yy];

}
if (xx == len0 && yy < len1) {

/* last col
*/
if (endgaps)

col1[yy] −= ins0+ins1*(len1−yy);

if(col1[yy] > smax) {

smax = col1[yy];
dmax = id;

}

}

}
if (endgaps && xx < len0)

col1[yy−1] −= ins0+ins1*(len0−xx);

if (col1[yy−1] > smax) {

smax = col1[yy−1];
dmax = id;

}
tmp = col0; col0 = col1; col1 = tmp;
}
(void) free((char *)ndely);
(void) free((char *)dely);
(void) free((char *)col0);
(void) free((char *)col1);

}

/*
*
* print() -- only routine visible outside this module
*
* static:
* getmat() -- trace back best path, count matches: print()
* pr_align() -- print alignment of described in array p[]: print()
* dumpblock() -- dump a block of lines with numbers, stars: pr_align()
* nums() -- put out a number line: dumpblock()
* putline() -- put out a line (name, [num], seq, [num]): dumpblock()
* stars() - -put a line of stars: dumpblock()
* stripname() -- strip any path and prefix from a seqname
*/
#include “nw.h”

| | | |
|---|---|---|
| #define SPC | 3 | |
| #define P_LINE | 256 | /* maximum output line */ |
| #define P_SPC | 3 | /* space between name or num and seq */ |

| | | |
|---|---|---|
| extern | _day[26][26]; | |
| int | olen; | /* set output line length */ |
| FILE | *fx; | /* output file */ |

| | |
|---|---|
| print() | print |
| { | |

| | |
|---|---|
| int | lx, ly, firstgap, lastgap; /* overlap */ |
| if ((fx = | fopen(ofile, “w”)) == 0) { |
| | fprintf(stderr, “%s: can't write %s\n”, prog, ofile); |
| | cleanup(1); |
| } | |

| fprintf(fx, “<first sequence: %s (length = %d)\n”, namex[0], len0); | |
|---|---|
| fprintf(fx, “<second sequence: %s (length = %d)\n”, namex[1], len1); | |
| olen = 60; | |
| lx = len0; | |
| ly = len1; | |
| firstgap = lastgap = 0; | |
| if (dmax < len1 − 1) { | /* leading gap in x */ |

pp[0].spc = firstgap = len1 − dmax − 1;
ly −= pp[0].spc;

| } | |
|---|---|
| else if (dmax > len1 − 1) { | /* leading gap in y */ |

pp[1].spc = firstgap = dmax − (len1 − 1);
lx −= pp[1].spc;

| } | |
|---|---|
| if (dmax0 < len0 − 1) { | /* trailing gap in x */ |

lastgap = len0 − dmax0 −1;
lx −= lastgap;

| } | |
|---|---|
| else if (dmax0 > len0 − 1) { | /* trailing gap in y */ |

lastgap = dmax0 − (len0 − 1);
ly −= lastgap;

}
getmat(lx, ly, firstgap, lastgap);
pr_align();

}
/*
* trace back the best path, count matches
*/
static

| | |
|---|---|
| getmat(lx, ly, firstgap, lastgap) | getmat |

| | | |
|---|---|---|
| int | lx, ly; | /* “core” (minus endgaps) */ |
| int | firstgap, lastgap; | /* leading trailing overlap */ |

{

| | |
|---|---|
| int | nm, i0, i1, siz0, siz1; |
| char | outx[32]; |
| double | pct; |
| register | n0, n1; |
| register char | *p0, *p1; |

/* get total matches, score
*/
i0 = i1 = siz0 = siz1 = 0;
p0 = seqx[0] + pp[1].spc;
p1 = seqx[1] + pp[0].spc;
n0 = pp[1].spc + 1;
n1 = pp[0].spc + 1;
nm = 0;
while ( *p0 && *p1 ) {

if (siz0) {

p1++;
n1++;
siz0−−;

}
else if (siz1) {

p0++;
n0++;
siz1−−;

}
else {

if (xbm[*p0−‘A’]&xbm[*p1−‘A’])

nm++;

if (n0++ == pp[0].x[i0])

siz0 = pp[0].n[i0++];

if (nl++ == pp[1].x[i1])

siz1 = pp[1].n[il++];

p0++;
p1++;

}

}
/* pct homology:
* if penalizing endgaps, base is the shorter seq
* else, knock off overhangs and take shorter core
*/
if (endgaps)

lx = (len0 < len1)? len0 : len1;

else

lx = (lx < ly)? lx : ly;

pct = 100.*(double)nm/(double)lx;
fprintf(fx, “\n”);
fprintf(fx, “<%d match%s in an overlap of %d: %.2f percent similarity\n”,

nm, (nm == 1)? “” : “es”, lx, pct);

| | |
|---|---|
| fprintf(fx, “<gaps in first sequence: %d”, gapx); | ...getmat |
| if (gapx) { | |

(void) sprintf(outx, “(%d %s%s)”,

ngapx, (dna)? “base”: “residue”, (ngapx == 1)? “”:“s”);

fprintf(fx, “%s”, outx);

fprintf(fx, “, gaps in second sequence: %d”, gapy);
if (gapy) {

(void) sprintf(outx, “(%d %s%s)”,

ngapy, (dna)? “base”:“residue”, (ngapy == 1)? “”:“s”);

fprintf(fx, “%s”, outx);

}
if (dna)

fprintf(fx,
“\n<score: %d (match = %d, mismatch = %d, gap penalty = %d + %d per base)\n”,
smax, DMAT, DMIS, DINS0, DINS1);

else

fprintf(fx,
“\n<score: %d (Dayhoff PAM 250 matrix, gap penalty = %d + %d per residue)\n”,
smax, PINS0, PINS1);

if (endgaps)

fprintf(fx,
“<endgaps penalized. left endgap: %d %s%s, right endgap: %d %s%s\n”,
firstgap, (dna)? “base” : “residue”, (firstgap == 1)? “” : “s”,
lastgap, (dna)? “base” : “residue”, (lastgap == 1)? “” : “s”);

else

fprintf(fx, “<endgaps not penalized\n”);

}

| | | |
|---|---|---|
| static | nm; | /* matches in core -- for checking */ |
| static | lmax; | /* lengths of stripped file names */ |
| static | ij[2]; | /* jmp index for a path */ |
| static | nc[2]; | /* number at start of current line */ |
| static | ni[2]; | /* current elem number -- for gapping */ |
| static | siz[2]; | |
| static char | *ps[2]; | /* ptr to current element */ |
| static char | *po[2]; | /* ptr to next output char slot */ |
| static char | out[2][P_LINE]; | /* output line */ |
| static char | star[P_LINE]; | /* set by stars() */ |
| /* | | |

* print alignment of described in struct path pp[]
*/
static

| | |
|---|---|
| pr_align() | pr_align |
| { | |

| | | |
|---|---|---|
| int | nn; | /* char count */ |
| int | more; | |
| register | i; | |

for (i = 0, lmax = 0; i < 2;i++) {

| nn = stripname(namex[i]); | |
|---|---|
| if (nn > | lmax) |

lmax = nn;

nc[i] = 1;
ni[i] = 1;
siz[i] = ij[i] = 0;
ps[i] = seqx[i];
po[i] = out[i];

}

| | |
|---|---|
| for (nn = nm = 0, more = 1; more;) { | ...pr_align |

for (i = more = 0; i < 2; i++) {

/*
* do we have more of this sequence?
*/
if (!*ps[i])

continue;

| more++; | |
|---|---|
| if (pp[i].spc) { | /* leading space */ |

*po[i]++ = ‘ ’;
pp[i].spc−−;

| } | |
|---|---|
| else if (siz[i]) { | /* in a gap */ |

*po[i]++ = ‘−’;
siz[i]−−;

| } | |
|---|---|
| else { | /* we're putting a seq element |

*/

*po[i] = *ps[i];
if (islower(*ps[i]))

*ps[i] = toupper(*ps[i]);

po[i]++;
ps[i]++;
/*
* are we at next gap for this seq?
*/
if (ni[i] == pp[i].x[ij[i]]) {

/*
* we need to merge all gaps
* at this location
*/
siz[i] == pp[i].n[ij[i]++];
while (ni[i] == pp[i].x[ij[i]])

siz[i] += pp[i].n[ij[i]++];

}
ni[i]++;

}

}
if (++nn == olen || !more && nn) {

dumpblock();
for (i = 0; i < 2; i++)

po[i] = out[i];

nn = 0;

}

}

}
/*
* dump a block of lines, including numbers, stars: pr_align()
*/
static

| | |
|---|---|
| dumpblock() | dumpblock |
| { | |

register i;
for(i = 0; i < 2; i++)

*po[i]−− = ‘\0’;

...dumpblock

(void) putc(‘\n’, fx);
for (i = 0; i < 2; i++) {

if (*out[i] && (*out[i] != ‘ ’ || *(po[i]) != ‘ ’)) {

if (i == 0)

nums(i);

if (i == 0 && *out[1])

stars();

putline(i);
if (i == 0 && *out[1])

fprintf(fx, star);

if (i == 1)

nums(i);

}

}

}
/*
* put out a number line: dumpblock()
*/
static

| | |
|---|---|
| nums(ix) | nums |

| | |
|---|---|
| int ix; | /* index in out[] holding seq line */ |

{

| | |
|---|---|
| char | nline[P_LINE]; |
| register | i, j; |
| register char | *pn, *px, *py; |

for(pn = nline, i = 0; i < lmax+P_SPC; i++, pn++)

*pn = ‘ ’;

for (i = nc[ix], py = out[ix]; *py; py++, pn++) {

if (*py == ‘ ’ || *py == ‘−’)

*pn = ‘ ’;

else {

if (i%10 == 0 || (i == 1 && nc[ix] != 1)) {

j = (i < 0)? −i : i;
for (px = pn; j; j/= 10, px−−)

*px = j%10 + ‘0’;

if (i < 0)

*px = ‘−’;

}
else

*pn = ‘ ’;

i++;

}

}
*pn = ‘\0’;
nc[ix] = i;
for (pn = nline; *pn; pn++)

(void) putc(*pn, fx);

(void) putc(‘\n’, fx);

}
/*
* put out a line (name, [num], seq. [num]): dumpblock()
*/
static

| | |
|---|---|
| putline(ix) | putline |

| | |
|---|---|
| int ix; | { |

...putline

| | |
|---|---|
| int | i; |
| register char | *px; |

for (px = namex[ix], i = 0; *px && *px != ‘:’; px++, i++)

(void) putc(*px, fx);

for (;i < lmax+P_SPC; i++)

(void) putc(‘ ’, fx);

/* these count from 1:
* ni[] is current element (from 1)
* nc[] is number at start of current line
*/
for (px = out[ix]; *px; px++)

(void) putc(*px&0x7F, fx);

(void) putc(‘\n’, fx);

}
/*
* put a line of stars (seqs always in out[0], out[1]): dumpblock()
*/
static

| | |
|---|---|
| stars() | stars |
| { | |

| | |
|---|---|
| int | i; |
| register char | *p0, *p1, cx, *px; |

| | |
|---|---|
| if | (!*out[0] \|\| (*out[0] == ‘ ’ && *(p0[0]) == ‘ ’) \|\| |
| | !*out[1] \|\| (*out[1] == ‘ ’ && *(po[1]) == ‘ ’)) |

return;

px = star;
for (i = lmax+P_SPC; i; i−−)

*px++ = ‘ ’;

for (p0 = out[0], p1 = out[1]; *p0 && *p1; p0++, p1++) {

if (isalpha(*p0) && isalpha(*p1)) {

if (xbm[*p0−‘A’]&xbm[*p1−‘A’]) {

cx = ‘*’;
nm++;

}
else if (!dna && _day[*p0− ‘A’][*p1−‘A’] > 0)

cx = ‘.’;

else

cx = ‘ ’;

}
else

cx = ‘ ’;

*px++ = cx;

}
*px++ = ‘\n’;
*px = ‘\0’;

}
/*
* strip path or prefix from pn, return len: pr_align()
*/
static

| | |
|---|---|
| stripname(pn) | stripname |

| | | |
|---|---|---|
| char | *pn; | /* file name (may be path) */ |

{

| | |
|---|---|
| register char | *px, *py; |

py = 0;
for (px = pn; *px; px++)

if (*px == ‘/’)

py = px + 1;

if (py)

(void) strcpy(pn, py);

return(strlen(pn));

}
/*
* cleanup() -- cleanup any tmp file
* getseq() -- read in seq, set dna, len, maxlen
* g_calloc() -- calloc() with error checkin
* readjmps() -- get the good jmps, from tmp file if necessary
* writejmps() -- write a filled array of jmps to a tmp file: nw()
*/
#include “nw.h”
#include <sys/file.h>

| | | |
|---|---|---|
| char | *jname = “/tmp/homgXXXXXX”; | /* tmp file for jmps */ |
| FILE | *fj; | |
| int | cleanup(); | /* cleanup tmp file */ |
| long | lseek(); | |
| /* | | |

* remove any tmp file if we blow
*/

| | |
|---|---|
| cleanup(i) | cleanup |

| | |
|---|---|
| int | i; |

{

if (fj)

(void) unlink(jname);

exit(i);

}
/*
* read, return ptr to seq, set dna, len, maxlen
* skip lines starting with ‘;’, ‘<’, or ‘>’
* seq in upper or lower case
*/
char *

| | |
|---|---|
| getseq(file, len) | getseq |

| | | |
|---|---|---|
| char | *file; | /* file name */ |
| int | *len; | /* seq len */ |

{

| | |
|---|---|
| char | line[1024], *pseq; |
| register char | *px, *py; |
| int | natgc, tlen; |
| FILE | *fp; |

if ((fp = fopen(file, “r”)) == 0) {

fprintf(stderr, “%s: can't read %s\n”, prog, file);
exit(1);

}
tlen = natgc = 0;
while (fgets(line, 1024, fp)) {

if (*line == ‘;’ || *line == ‘<’ || *line == ‘>’)

continue;

for (px = line; *px != ‘\n’; px++)

if (isupper(*px) || islower(*px))

tlen++;

}
if ((pseq = malloc((unsigned)(tlen+6))) == 0) {

fprintf(stderr, “%s: malloc() failed to get %d bytes for %s\n”, prog, tlen+6, file);
exit(1);

}
pseq[0] = pseq[1] = pseq[2] = pseq[3] = ‘\0’;

...getseq

py = pseq + 4;
*len = tlen;
rewind(fp);
while (fgets(line, 1024, fp)) {

if (*line == ‘;’ || *line == ‘<’ || *line == ‘>’)

continue;

for (px = line; *px != ‘\n’; px++) {

if (isupper(*px))

*py++ = *px;

else if (islower(*px))

*py++ = toupper(*px);

if (index(“ATGCU”, *(py−1)))

natgc++;

}

}
*py++ = ‘\0’;
*py = ‘\0’;
(void) fclose(fp);
dna = natgc > (tlen/3);
return(pseq+4);

}
char *

| | |
|---|---|
| g_calloc(msg, nx, sz) | g_calloc |

| | | |
|---|---|---|
| char | *msg; | /* program, calling routine */ |
| int | nx, sz; | /* number and size of elements */ |

{

| | |
|---|---|
| char | *px, *calloc(); |

if ((px = calloc((unsigned)nx, (unsigned)sz)) == 0) {

if (*msg) {

fprintf(stderr, “%s: g_calloc() failed %s (n= %d, sz= %d)\n”, prog, msg, nx, sz);
exit(1);

}

}
return(px);

}
/*
* get final jmps from dx[] or tmp file, set pp[], reset dmax: main()
*/

| | |
|---|---|
| readjmps() | readjmps |
| { | |

| | |
|---|---|
| int | fd = −1; |
| int | siz, i0, i1; |

register i, j, xx;
if (fj) {

| (void) fclose(fj); | |
|---|---|
| if ((fd = | open(jname, O_RDONLY, 0)) < 0) { |

fprintf(stderr, “%s: can't open() %s\n”, prog, jname);
cleanup(1);

}

}
for (i = i0 = i1 = 0, dmax0 = dmax, xx = len0; ;i++) {

while (1) {

for (j = dx[dmax].ijmp; j >= 0 && dx[dmax].jp.x[j] >= xx; j−−)

;

...readjmps

if (j < 0 && dx[dmax].offset && fj) {

(void) lseek(fd, dx[dmax].offset, 0);
(void) read(fd, (char *)&dx[dmax].jp, sizeof(struct jmp));
(void) read(fd, (char *)&dx[dmax].offset, sizeof(dx[dmax].offset));
dx[dmax].ijmp = MAXJMP−1;

}
else

break;

}
if (i >= JMPS) {

fprintf(stderr, “%s: too many gaps in alignment\n”, prog);
cleanup(1);

}
if (j >= 0) {

| siz = dx[dmax].jp.n[j]; | |
|---|---|
| xx = dx[dmax].jp.x[j]; | |
| dmax += siz; | |
| if (siz < 0) { | /* gap in second seq */ |

pp[1].n[il] = −siz;
xx += siz;
/* id = xx − yy + len1 − 1
*/
pp[1].x[il] = xx − dmax + len1 − 1;
gapy++;
ngapy −= siz;

/* ignore MAXGAP when doing endgaps */

siz = (−siz < MAXGAP || endgaps)? −siz : MAXGAP;
il++;

| } | |
|---|---|
| else if (siz > 0) { | /* gap in first seq */ |

pp[0] .n[i0] = siz;
pp[0] .x[i0] = xx;
gapx++;
ngapx += siz;

/* ignore MAXGAP when doing endgaps */

siz = (siz < MAXGAP || endgaps)? siz : MAXGAP;
i0++;

}

}
else

break;

}
/* reverse the order of jmps
*/
for (j = 0, i0−−; j < i0; j++, i0−−) {

i = pp[0].n[j]; pp[0].n[j] = pp[0].n[i0]; pp[0].n[i0] = i;
i = pp[0].x[j]; pp[0].x[j] = pp[0].x[i0]; pp[0].x[i0] = i;

}
for (j = 0, i1−−; j < i1; j++, i1−−) {

i = pp[1].n[j]; pp[1].n[j] = pp[1].n[i1]; pp[1].n[i1] = i;
i = pp[1].x[j]; pp[1].x[j] = pp[1].x[i1]; pp[1].x[i1] = i;

}
if (fd >= 0)

(void) close(fd);

if (fj) {

(void) unlink(jname);
fj = 0;
offset = 0;

| | |
|---|---|
| } | } |

/*
* write a filled jmp struct offset of the prev one (if any): nw()
*/

| | |
|---|---|
| writejmps(ix) | writejmps |

| | |
|---|---|
| int | ix; |

{

| | |
|---|---|
| char | *mktemp(); |

if (!fj) {

if (mktemp(jname) < 0) {

fprintf(stderr, “%s: can't mktemp() %s\n”, prog, jname);
cleanup(1);

}
if ((fj = fopen(jname, “w”)) == 0) {

fprintf(stderr, “%s: can't write %s\n”, prog, jname);
exit(1);

}

}
(void) fwrite((char *)&dx[ix].jp, sizeof(struct jmp), 1, fj);
(void) fwrite((char *)&dx[ix].offset, sizeof(dx[ix].offset), 1, fj);

}

TABLE 2

| | | |
|---|---|---|
| PRO | XXXXXXXXXXXXXXX | (Length = 15 amino acids) |
| Comparison | XXXXXYYYYYYY | (Length = 12 amino acids) |
| Protein | | |

% amino acid sequence identity = (the number of identically matching amino acid residues between the two polypeptide sequences as determined by ALIGN-2) divided by (the total number of amino acid residues of the PRO polypeptide) = 5 divided by 15 = 33.3%

TABLE 3

| | | |
|---|---|---|
| PRO | XXXXXXXXXX | (Length = 10 amino acids) |
| Comparison | XXXXXYYYYYYZZYZ | (Length = 15 amino acids) |
| Protein | | |

% amino acid sequence identity = (the number of identically matching amino acid residues between the two polypeptide sequences as determined by ALIGN-2) divided by (the total number of amino acid residues of the PRO polypeptide) = 5 divided by 10 = 50%

TABLE 4

| | | |
|---|---|---|
| PRO-DNA | NNNNNNNNNNNNNN | (Length = 14 nucleotides) |
| Comparison | NNNNNNLLLLLLLLLL | (Length = 16 nucleotides) |
| DNA | | |

% nucleic acid sequence identity = (the number of identically matching nucleotides between the two nucleic acid sequences as determined by ALIGN-2) divided by (the total number of nucleotides of the PRO-DNA nucleic acid sequence) = 6 divided by 14 = 42.9%

TABLE 5

| | | |
|---|---|---|
| PRO-DNA | NNNNNNNNNNNN | (Length = 12 nucleotides) |
| Comparison | NNNNLLLVV | (Length = 9 nucleotides) |
| DNA | | |

% nucleic acid sequence identity = (the number of identically matching nucleotides between the two nucleic acid sequences as determined by ALIGN-2) divided by (the total number of nucleotides of the PRO-DNA nucleic acid sequence) = 4 divided by 12 = 33.3%

II. Composition and Methods of the Invention Full-Length PRO Polypetides

A. Full-Length PRO Polypeptides

The present invention provides newly identified and isolated nucleotide sequences encoding polypeptides referred to in the present application as PRO polypeptides. In particular, cDNAs encoding various PRO polypeptides have been identified and isolated, as disclosed in further detail in the Examples below. It is noted that proteins produced in separate expression rounds may be given different PRO numbers but the UNQ number is unique for any given DNA and the encoded protein, and will not be changed. However, for sake of simplicity, in the present specification the protein encoded by the full length native nucleic acid molecules disclosed herein as well as all further native homologues and variants included in the foregoing definition of PRO, will be referred to as “PRO/number”, regardless of their origin or mode of preparation.

As disclosed in the Examples below, various cDNA clones have been deposited with the ATCC. The actual nucleotide sequences of those clones can readily be determined by the skilled artisan by sequencing of the deposited clone using routine methods in the art. The predicted amino acid sequence can be determined from the nucleotide sequence using routine skill. For the PRO polypeptides and encoding nucleic acids described herein, Applicants have identified what is believed to be the reading frame best identifiable with the sequence information available at the time.

B. PRO Polypeptide Variants

In addition to the full-length native sequence PRO polypeptides described herein, it is contemplated that PRO variants can be prepared. PRO variants can be prepared by introducing appropriate nucleotide changes into the PRO DNA, and/or by synthesis of the desired PRO polypeptide. Those skilled in the art will appreciate that amino acid changes may alter post-translational processes of the PRO, such as changing the number or position of glycosylation sites or altering the membrane anchoring characteristics.

Variations in the native full-length sequence PRO or in various domains of the PRO described herein, can be made, for example, using any of the techniques and guidelines for conservative and non-conservative mutations set forth, for instance, in U.S. Pat. No. 5,364,934. Variations may be a substitution, deletion or insertion of one or more codons encoding the PRO that results in a change in the amino acid sequence of the PRO as compared with the native sequence PRO. Optionally the variation is by substitution of at least one amino acid with any other amino acid in one or more of the domains of the PRO. Guidance in determining which amino acid residue may be inserted, substituted or deleted without adversely affecting the desired activity may be found by comparing the sequence of the PRO with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

PRO polypeptide fragments are provided herein. Such fragments may be truncated at the N-terminus or C-terminus, or may lack internal residues, for example, when compared with a full length native protein. Certain fragments lack amino acid residues that are not essential for a desired biological activity of the PRO polypeptide.

PRO fragments may be prepared by any of a number of conventional techniques. Desired peptide fragments may be chemically synthesized. An alternative approach involves generating PRO fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, PRO polypeptide fragments share at least one biological and/or immunological activity with the native PRO polypeptide disclosed herein.

In particular embodiments, conservative substitutions of interest are shown in Table 6 under the heading of preferred substitutions. If such substitutions result in a change in biological activity, then more substantial changes, denominated exemplary substitutions in Table 6, or as further described below in reference to amino acid classes, are introduced and the products screened.

TABLE 6

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in function or immunological identity of the PRO polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gin, his, lys, arg;

(5) residues that influence chain orientation: gly, pro; and

(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene 34:315 (1985)], restriction selection mutagenesis [Wells et al., Philos. Trans. R. Soc. London SerA, 317:415 (1986)] or other known techniques can be performed on the cloned DNA to produce the PRO variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins, (W. H. Freeman & Co., N.Y.); Chothia, J. Mol. Biol., 150:1 (1976)]. If alanine substitution does not yield adequate amounts of variant, an isoteric amino acid can be used.

C. Modifications of PRO

Covalent modifications of PRO are included within the scope of this invention. One type of covalent modification includes reacting targeted amino acid residues of a PRO polypeptide with an organic derivatizing agent that is capable of reacting with selected side chains or the N- or C- terminal residues of the PRO. Derivatization with bifunctional agents is useful, for instance, for crosslinking PRO to a water-insoluble support matrix or surface for use in the method for purifying anti-PRO antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate),bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of the PRO polypeptide included within the scope of this invention comprises altering the native glycosylation pattern of the polypeptide. “Altering the native glycosylation pattern” is intended for purposes herein to mean deleting one or more carbohydrate moieties found in native sequence PRO (either by removing the underlying glycosylation site or by deleting the glycosylation by chemical and/or enzymatic means), and/or adding one or more glycosylation sites that are not present in the native sequence PRO. In addition, the phrase includes qualitative changes in the glycosylation of the native proteins, involving a change in the nature and proportions of the various carbohydrate moieties present.

Addition of glycosylation sites to the PRO polypeptide may be accomplished by altering the amino acid sequence. The alteration may be made, for example, by the addition of, or substitution by, one or more serine or threonine residues to the native sequence PRO (for O-linked glycosylation sites). The PRO amino acid sequence may optionally be altered through changes at the DNA level, particularly by mutating the DNA encoding the PRO polypeptide at preselected bases such that codons are generated that will translate into the desired amino acids.

Another means of increasing the number of carbohydrate moieties on the PRO polypeptide is by chemical or enzymatic coupling of glycosides to the polypeptide. Such methods are described in the art, e.g., in WO 87/05330 published Sep. 11, 1987, and in Aplin and Wriston, CRC Crit. Rev. Biochem., pp. 259-306 (1981).

Removal of carbohydrate moieties present on the PRO polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of PRO comprises linking the PRO polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The PRO of the present invention may also be modified in a way to form a chimeric molecule comprising PRO fused to another, heterologous polypeptide or amino acid sequence.

In one embodiment, such a chimeric molecule comprises a fusion of the PRO with a tag polypeptide which provides an epitope to which an anti-tag antibody can selectively bind. The epitope tag is generally placed at the amino- or carboxyl- terminus of the PRO. The presence of such epitope-tagged forms of the PRO can be detected using an antibody against the tag polypeptide. Also, provision of the epitope tag enables the PRO to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule may comprise a fusion of the PRO with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a PRO polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995.

D. Preparation of PRO

The description below relates primarily to production of PRO by culturing cells transformed or transfected with a vector containing PRO nucleic acid. It is, of course, contemplated that alternative methods, which are well known in the art, may be employed to prepare PRO. For instance, the PRO sequence, or portions thereof, may be produced by direct peptide synthesis using solid-phase techniques [see, e.g., Stewart et al., Solid-Phase Peptide Synthesis, W. H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the PRO may be chemically synthesized separately and combined using chemical or enzymatic methods to produce the full-length PRO.

1. Isolation of DNA Encoding PRO

DNA encoding PRO may be obtained from a cDNA library prepared from tissue believed to possess the PRO mRNA and to express it at a detectable level. Accordingly, human PRO DNA can be conveniently obtained from a cDNA library prepared from human tissue, such as described in the Examples. The PRO-encoding gene may also be obtained from a genomic library or by known synthetic procedures (e.g., automated nucleic acid synthesis).

Libraries can be screened with probes (such as antibodies to the PRO or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding PRO is to use PCR methodology [Sambrook et al., supra; Dieffenbach et al., PCR Primer: A Laboratory Manual (Cold Spring Harbor Laboratory Press, 1995)].

The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for PRO production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published Jun. 29, 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA ; E. coli W3110 strain 9E4, which has the complete genotype tonA ; E. coli W3110 strain 27C7 (ATCC 55,244), which has the complete genotype tonA ptr3 phoA E15 (argF-lac)169degP ompT kan^{r}; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued Aug. 7, 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for PRO-encoding vectors. Saccharomyces cerevisiae is a commonly used lower eukaryotic host microorganism. Others include Schizosaccharomyces pombe (Beach and Nurse, Nature, 290:140 [1981]; EP 139,383 published May 2, 1985); Kluyveromyces hosts (U.S. Pat. No. 4,943,529; Fleer et al., Bio/Technology, 9:968-975(1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24, 178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published Oct. 31, 1990); and filamentous fungi such as, e.g., Neurospora, Penicillium, Tolypocladium (WO 91/00357 published Jan. 10, 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81:1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated PRO are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen. Virol., 36:59 (1977)); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

3. Selection and Use of a Replicable Vector

The nucleic acid (e.g., cDNA or genomic DNA) encoding PRO may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The PRO may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the PRO-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published Apr. 4, 1990), or the signal described in WO 90/13646 published Nov. 15, 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the PRO-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the PRO-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding PRO.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

PRO transcription from vectors in mammalian host cells is controlled, for example, by promoters obtained from the genomes of viruses such as polyoma virus, fowlpox virus (UK 2,211,504 published Jul. 5, 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the PRO by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the PRO coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding PRO.

Still other methods, vectors, and host cells suitable for adaptation to the synthesis of PRO in recombinant vertebrate cell culture are described in Gething et al., Nature, 293:620-625 (1981); Mantei et al., Nature, 281:40-46 (1979); EP 117,060; and EP 117,058.

4. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence PRO polypeptide or against a synthetic peptide based on the DNA sequences provided herein or against exogenous sequence fused to PRO DNA and encoding a specific antibody epitope.

5. Purification of Polypeptide

Forms of PRO may be recovered from culture medium or from host cell lysates. If membrane-bound, it can be released from the membrane using a suitable detergent solution (e.g. Triton-X 100) or by enzymatic cleavage. Cells employed in expression of PRO can be disrupted by various physical or chemical means, such as freeze-thaw cycling, sonication, mechanical disruption, or cell lysing agents.

It may be desired to purify PRO from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the PRO. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular PRO produced.

E. Uses for PRO

Nucleotide sequences (or their complement) encoding PRO have various applications in the art of molecular biology, including uses as hybridization probes, in chromosome and gene mapping and in the generation of anti-sense RNA and DNA. PRO nucleic acid will also be useful for the preparation of PRO polypeptides by the recombinant techniques described herein.

The full-length native sequence PRO gene, or portions thereof, may be used as hybridization probes for a cDNA library to isolate the full-length PRO cDNA or to isolate still other cDNAs (for instance, those encoding naturally-occurring variants of PRO or PRO from other species) which have a desired sequence identity to the native PRO sequence disclosed herein. Optionally, the length of the probes will be about 20 to about 50 bases. The hybridization probes may be derived from at least partially novel regions of the full length native nucleotide sequence wherein those regions may be determined without undue experimentation or from genomic sequences including promoters, enhancer elements and introns of native sequence PRO. By way of example, a screening method will comprise isolating the coding region of the PRO gene using the known DNA sequence to synthesize a selected probe of about 40 bases. Hybridization probes may be labeled by a variety of labels, including radionucleotides such as ³²P or ³⁵S, or enzymatic labels such as alkaline phosphatase coupled to the probe via avidin/biotin coupling systems. Labeled probes having a sequence complementary to that of the PRO gene of the present invention can be used to screen libraries of human cDNA, genomic DNA or mRNA to determine which members of such libraries the probe hybridizes to. Hybridization techniques are described in further detail in the Examples below.

Any EST sequences disclosed in the present application may similarly be employed as probes, using the methods disclosed herein.

Other useful fragments of the PRO nucleic acids include antisense or sense oligonucleotides comprising a singe-stranded nucleic acid sequence (either RNA or DNA) capable of binding to target PRO mRNA (sense) or PRO DNA (antisense) sequences. Antisense or sense oligonucleotides, according to the present invention, comprise a fragment of the coding region of PRO DNA. Such a fragment generally comprises at least about 14 nucleotides, preferably from about 14 to 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. The antisense oligonucleotides thus may be used to block expression of PRO proteins. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Other examples of sense or antisense oligonucleotides include those oligonucleotides which are covalently linked to organic moieties, such as those described in WO 90/10048, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, CaPO₄-mediated DNA transfection, electroporation, or by using gene transfer vectors such as Epstein-Barr virus. In a preferred procedure, an antisense or sense oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MuLV, N2 (a retrovirus derived from M-MuLV), or the double copy vectors designated DCT5A, DCT5B and DCT5C (see WO 90/13641).

Sense or antisense oligonucleotides also may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

Antisense or sense RNA or DNA molecules are generally at least about 5 bases in length, about 10 bases in length, about 15 bases in length, about 20 bases in length, about 25 bases in length, about 30 bases in length, about 35 bases in length, about 40 bases in length, about 45 bases in length, about 50 bases in length, about 55 bases in length, about 60 bases in length, about 65 bases in length, about 70 bases in length, about 75 bases in length, about 80 bases in length, about 85 bases in length, about 90 bases in length, about 95 bases in length, about 100 bases in length, or more.

The probes may also be employed in PCR techniques to generate a pool of sequences for identification of closely related PRO coding sequences.

Nucleotide sequences encoding a PRO can also be used to construct hybridization probes for mapping the gene which encodes that PRO and for the genetic analysis of individuals with genetic disorders. The nucleotide sequences provided herein may be mapped to a chromosome and specific regions of a chromosome using known techniques, such as in situ hybridization, linkage analysis against known chromosomal markers, and hybridization screening with libraries.

When the coding sequences for PRO encode a protein which binds to another protein (example, where the PRO is a receptor), the PRO can be used in assays to identify the other proteins or molecules involved in the binding interaction. By such methods, inhibitors of the receptor/ligand binding interaction can be identified. Proteins involved in such binding interactions can also be used to screen for peptide or small molecule inhibitors or agonists of the binding interaction. Also, the receptor PRO can be used to isolate correlative ligand(s). Screening assays can be designed to find lead compounds that mimic the biological activity of a native PRO or a receptor for PRO. Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including protein-protein binding assays, biochemical screening assays, immunoassays and cell based assays, which are well characterized in the art.

Nucleic acids which encode PRO or its modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, cDNA encoding PRO can be used to clone genomic DNA encoding PRO in accordance with established techniques and the genomic sequences used to generate transgenic animals that contain cells which express DNA encoding PRO. Methods for generating transgenic animals, particularly animals such as mice or rats, have become conventional in the art and are described, for example, in U.S. Pat. Nos. 4,736,866 and 4,870,009. Typically, particular cells would be targeted for PRO transgene incorporation with tissue-specific enhancers. Transgenic animals that include a copy of a transgene encoding PRO introduced into the germ line of the animal at an embryonic stage can be used to examine the effect of increased expression of DNA encoding PRO. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of PRO can be used to construct a PRO “knock out” animal which has a defective or altered gene encoding PRO as a result of homologous recombination between the endogenous gene encoding PRO and altered genomic DNA encoding PRO introduced into an embryonic stem cell of the animal. For example, cDNA encoding PRO can be used to clone genomic DNA encoding PRO in accordance with established techniques. A portion of the genomic DNA encoding PRO can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the PRO polypeptide.

Nucleic acid encoding the PRO polypeptides may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83:4143-4146 [1986]). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 [1993]). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

The PRO polypeptides described herein may also be employed as molecular weight markers for protein electrophoresis purposes and the isolated nucleic acid sequences may be used for recombinantly expressing those markers.

The nucleic acid molecules encoding the PRO polypeptides or fragments thereof described herein are useful for chromosome identification. In this regard, there exists an ongoing need to identify new chromosome markers, since relatively few chromosome marking reagents, based upon actual sequence data are presently available. Each PRO nucleic acid molecule of the present invention can be used as a chromosome marker.

The PRO polypeptides and nucleic acid molecules of the present invention may also be used diagnostically for tissue typing, wherein the PRO polypeptides of the present invention may be differentially expressed in one tissue as compared to another, preferably in a diseased tissue as compared to a normal tissue of the same tissue type. PRO nucleic acid molecules will find use for generating probes for PCR, Northern analysis, Southern analysis and Western analysis.

The PRO polypeptides described herein may also be employed as therapeutic agents. The PRO polypeptides of the present invention can be formulated according to known methods to prepare pharmaceutically useful compositions, whereby the PRO product hereof is combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™ or PEG.

The formulations to be used for in vivo administration must be sterile. This is readily accomplished by filtration through sterile filtration membranes, prior to or following lyophilization and reconstitution.

Therapeutic compositions herein generally are placed into a container having a sterile access port, for example, an intravenous solution bag or vial having a stopper pierceable by a hypodermic injection needle.

The route of administration is in accord with known methods, e.g. injection or infusion by intravenous, intraperitoneal, intracerebral, intramuscular, intraocular, intraarterial or intralesional routes, topical administration, or by sustained release systems.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

When in vivo administration of a PRO polypeptide or agonist or antagonist thereof is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue.

Where sustained-release administration of a PRO polypeptide is desired in a formulation with release characteristics suitable for the treatment of any disease or disorder requiring administration of the PRO polypeptide, microencapsulation of the PRO polypeptide is contemplated. Microencapsulation of recombinant proteins for sustained release has been successfully performed with human growth hormone (rhGH), interferon-(rhIFN-), interleukin-2, and MN rgp120. Johnson et al., Nat. Med., 2:795-799(1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

The sustained-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

This invention encompasses methods of screening compounds to identify those that mimic the PRO polypeptide (agonists) or prevent the effect of the PRO polypeptide (antagonists). Screening assays for antagonist drug candidates are designed to identify compounds that bind or complex with the PRO polypeptides encoded by the genes identified herein, or otherwise interfere with the interaction of the encoded polypeptides with other cellular proteins. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

All assays for antagonists are common in that they call for contacting the drug candidate with a PRO polypeptide encoded by a nucleic acid identified herein under conditions and for a time sufficient to allow these two components to interact.

In binding assays, the interaction is binding and the complex formed can be isolated or detected in the reaction mixture. In a particular embodiment, the PRO polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the PRO polypeptide and drying. Alternatively, an immobilized antibody, e.g., a monoclonal antibody, specific for the PRO polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular PRO polypeptide encoded by a gene identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding a PRO polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the PRO polypeptide may be added to a cell along with the compound to be screened for a particular activity and the ability of the compound to inhibit the activity of interest in the presence of the PRO polypeptide indicates that the compound is an antagonist to the PRO polypeptide. Alternatively, antagonists may be detected by combining the PRO polypeptide and a potential antagonist with membrane-bound PRO polypeptide receptors or recombinant receptors under appropriate conditions for a competitive inhibition assay. The PRO polypeptide can be labeled, such as by radioactivity, such that the number of PRO polypeptide molecules bound to the receptor can be used to determine the effectiveness of the potential antagonist. The gene encoding the receptor can be identified by numerous methods known to those of skill in the art, for example, ligand panning and FACS sorting. Coligan et al., Current Protocols in Immun., 1(2): Chapter 5 (1991). Preferably, expression cloning is employed wherein polyadenylated RNA is prepared from a cell responsive to the PRO polypeptide and a cDNA library created from this RNA is divided into pools and used to transfect COS cells or other cells that are not responsive to the PRO polypeptide. Transfected cells that are grown on glass slides are exposed to labeled PRO polypeptide. The PRO polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for receptor identification, labeled PRO polypeptide can be photoaffinity-linked with cell membrane or extract preparations that express the receptor molecule. Cross-linked material is resolved by PAGE and exposed to X-ray film. The labeled complex containing the receptor can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative receptor.

In another assay for antagonists, mammalian cells or a membrane preparation expressing the receptor would be incubated with labeled PRO polypeptide in the presence of the candidate compound. The ability of the compound to enhance or block this interaction could then be measured.

More specific examples of potential antagonists include an oligonucleotide that binds to the fusions of immunoglobulin with PRO polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist may be a closely related protein, for example, a mutated form of the PRO polypeptide that recognizes the receptor but imparts no effect, thereby competitively inhibiting the action of the PRO polypeptide.

Another potential PRO polypeptide antagonist is an antisense RNA or DNA construct prepared using antisense technology, where, e.g., an antisense RNA or DNA molecule acts to block directly the translation of mRNA by hybridizing to targeted mRNA and preventing protein translation. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature PRO polypeptides herein, is used to design an antisense RNA oligonucleotide of from about 10 to 40 base pairs in length. A DNA oligonucleotide is designed to be complementary to a region of the gene involved in transcription (triple helix—see Lee et al., Nucl. Acids Res., 6:3073 (1979); Cooney et al., Science, 241: 456 (1988); Dervan et al., Science, 251:1360 (1991)), thereby preventing transcription and the production of the PRO polypeptide. The antisense RNA oligonucleotide hybridizes to the mRNA in vivo and blocks translation of the mRNA molecule into the PRO polypeptide (antisense—Okano, Neurochem., 56:560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA may be expressed in vivo to inhibit production of the PRO polypeptide. When antisense DNA is used, oligodeoxyribonucleotides derived from the translation-initiation site, e.g., between about −10 and +10 positions of the target gene nucleotide sequence, are preferred.

Potential antagonists include small molecules that bind to the active site, the receptor binding site, or growth factor or other relevant binding site of the PRO polypeptide, thereby blocking the normal biological activity of the PRO polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology, 4:469-471 (1994), and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

These small molecules can be identified by any one or more of the screening assays discussed hereinabove and/or by any other screening techniques well known for those skilled in the art.

Diagnostic and therapeutic uses of the herein disclosed molecules may also be based upon the positive functional assay hits disclosed and described below.

F. Anti-PRO Antibodies

The present invention further provides anti-PRO antibodies. Exemplary antibodies include polyclonal, monoclonal, humanized, bispecific, and heteroconjugate antibodies.

1. Polyclonal Antibodies

The anti-PRO antibodies may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the PRO polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

2. Monoclonal Antibodies

The anti-PRO antibodies may, alternatively, be monoclonal antibodies. Monoclonal antibodies may be prepared using hybridoma methods, such as those described by Kohler and Milstein, Nature 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the PRO polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against PRO. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

3. Human and Humanized Antibodies

The anti-PRO antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol. 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boemer et al. are also available for the preparation of human monoclonal antibodies [Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

The antibodies may also be affinity matured using known selection and/or mutagenesis methods as described above. Preferred affinity matured antibodies have an affinity which is five times, more preferably 10 times, even more preferably 20 or 30 times greater than the starting antibody (generally murine, humanized or human) from which the matured antibody is prepared.

4. Bispecific Antibodies

Bispecific antibodies are monoclonal, preferably human or humanized, antibodies that have binding specificities for at least two different antigens. In the present case, one of the binding specificities is for the PRO, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature, 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various technique for making and isolating bispecific antibody fragments directly from recombinant cell culture have also been described. For example, bispecific antibodies have been produced using leucine zippers. Kostelny et al, J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al, J. Immunol. 152:5368 (1994). Antibodies with more than two valencies are contemplated. For example, trispecific antibodies can be prepared. Tutt et al., J. Immunol. 147:60 (1991).

Exemplary bispecific antibodies may bind to two different epitopes on a given PRO polypeptide herein. Alternatively, an anti-PRO polypeptide arm may be combined with an arm which binds to a triggering molecule on a leukocyte such as a T-cell receptor molecule (e.g. CD2, CD3, CD28, or B7), or Fc receptors for IgG (FcγR), such asFcγRI (CD64), FcγRII (CD32) and FcγRIII (CD16) so as to focus cellular defense mechanisms to the cell expressing the particular PRO polypeptide. Bispecific antibodies may also be used to localize cytotoxic agents to cells which express a particular PRO polypeptide. These antibodies possess a PRO-binding arm and an arm which binds a cytotoxic agent or a radionuclide chelator, such as EOTUBE, DPTA, DOTA, or TETA. Another bispecific antibody of interest binds the PRO polypeptide and further binds tissue factor (TF).

5. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

6. Effector Function Engineering

It may be desirable to modify the antibody of the invention with respect to effector function, so as to enhance, e.g., the effectiveness of the antibody in treating cancer. For example, cysteine residue(s) may be introduced into the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp. Med., 176: 1191-1195 (1992) and Shopes, J. Immunol., 148: 2918-2922 (1992). Homodimeric antibodies with enhanced anti-tumor activity may also be prepared using heterobifunctional cross-linkers as described in Wolff et al. Cancer Research, 53: 2560-2565 (1993). Alternatively, an antibody can be engineered that has dual Fc regions and may thereby have enhanced complement lysis and ADCC capabilities. See Stevenson et al., Anti-Cancer Drug Design. 3: 219-230 (1989).

7. Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutareldehyde), bis-azido compounds (such as bis (p-azidobenzoyl) hexanediamine), bis-diazoniumderivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody may be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

8. Immunoliposomes

The antibodies disclosed herein may also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

9. Pharmaceutical Compositions of Antibodies

Antibodies specifically binding a PRO polypeptide identified herein, as well as other molecules identified by the screening assays disclosed hereinbefore, can be administered for the treatment of various disorders in the form of pharmaceutical compositions.

If the PRO polypeptide is intracellular and whole antibodies are used as inhibitors, internalizing antibodies are preferred. However, lipofections or liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence.. Such peptides can be synthesized chemically and/or produced by recombinant DNA technology. See, e.g., Marasco et al., Proc. Natl. Acad. Sci. USA, 90: 7889-7893 (1993). The formulation herein may also contain more than one active compound as necessary for the particular indication being treated, preferably those with complementary activities that do not adversely affect each other. Alternatively, or in addition, the composition may comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

G. Uses for anti-PRO Antibodies

The anti-PRO antibodies of the invention have various utilities. For example, anti-PRO antibodies may be used in diagnostic assays for PRO, e.g., detecting its expression (and in some cases, differential expression) in specific cells, tissues, or serum. Various diagnostic assay techniques known in the art may be used, such as competitive binding assays, direct or indirect sandwich assays and immunoprecipitation assays conducted in either heterogeneous or homogeneous phases [Zola, Monoclonal Antibodies: A Manual of Techniques, CRC Press, Inc. (1987) pp. 147-158]. The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Anti-PRO antibodies also are useful for the affinity purification of PRO from recombinant cell culture or natural sources. In this process, the antibodies against PRO are immobilized on a suitable support, such a Sephadex resin or filter paper, using methods well known in the art. The immobilized antibody then is contacted with a sample containing the PRO to be purified, and thereafter the support is washed with a suitable solvent that will remove substantially all the material in the sample except the PRO, which is bound to the immobilized antibody. Finally, the support is washed with another suitable solvent that will release the PRO from the antibody.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

### Extracellular Domain Homology Screening to Identify Novel Polypeptides and cDNA Encoding Therefor

The extracellular domain (ECD) sequences (including the secretion signal sequence, if any) from about 950 known secreted proteins from the Swiss-Protpublic database were used to search EST databases. The EST databases included public databases (e.g., Dayhoff, GenBank), and proprietary databases (e.g. LIFESEQ™, Incyte Pharmaceuticals, Palo Alto, Calif.). The search was performed using the computer program BLAST or BLAST-2 (Altschul et al., Methods in Enzymology. 266:460-480 (1996)) as a comparison of the ECD protein sequences to a 6 frame translation of the EST sequences. Those comparisons with a BLAST score of 70 (or in some cases 90) or greater that did not encode known proteins were clustered and assembled into consensus DNA sequences with the program “phrap” (Phil Green, University of Washington, Seattle, Wash.).

Using this extracellular domain homology screen, consensus DNA sequences were assembled relative to the other identified EST sequences using phrap. In addition, the consensus DNA sequences obtained were often (but not always) extended using repeated cycles of BLAST or BLAST-2 and phrap to extend the consensus sequence as far as possible using the sources of EST sequences discussed above.

Based upon the consensus sequences obtained as described above, oligonucleotides were then synthesized and used to identify by PCR a cDNA library that contained the sequence of interest and for use as probes to isolate a clone of the full-length coding sequence for a PRO polypeptide. Forward and reverse PCR primers generally range from 20 to 30 nucleotides and are often designed to give a PCR product of about 100-1000 bp in length. The probe sequences are typically 40-55 bp in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, with the PCR primer pair. A positive library was then used to isolate clones encoding the gene of interest using the probe oligonucleotide and one of the primer pairs.

The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

### Example 2

### Isolation of cDNA Clones by Amylase Screening

1. Preparation of Oligo dT Primed cDNA Library

mRNA was isolated from a human tissue of interest using reagents and protocols from Invitrogen, San Diego, Calif. (Fast Track 2). This RNA was used to generate an oligo dT primed cDNA library in the vector pRK5D using reagents and protocols from Life Technologies, Gaithersburg, Md. (Super Script Plasmid System). In this procedure, the double stranded cDNA was sized to greater than 1000 bp and the SalI/NotI linkered cDNA was cloned into XhoI/NotI cleaved vector. pRK5D is a cloning vector that has an sp6 transcription initiation site followed by an SfiI restriction enzyme site preceding the XhoI/NotI cDNA cloning sites.

2. Preparation of Random Primed cDNA Library

A secondary cDNA library was generated in order to preferentially represent the 5′ ends of the primary cDNA clones. Sp6 RNA was generated from the primary library (described above), and this RNA was used to generate a random primed cDNA library in the vector pSST-AMY.0 using reagents and protocols from Life Technologies (Super Script Plasmid System, referenced above). In this procedure the double stranded cDNA was sized to 500-1000 bp, Tinkered with blunt to NotI adaptors, cleaved with SfiI, and cloned into SfiI/NotI cleaved vector. pSST-AMY.0 is a cloning vector that has a yeast alcohol dehydrogenase promoter preceding the cDNA cloning sites and the mouse amylase sequence (the mature sequence without the secretion signal) followed by the yeast alcohol dehydrogenase terminator, after the cloning sites. Thus, cDNAs cloned into this vector that are fused in frame with amylase sequence will lead to the secretion of amylase from appropriately transfected yeast colonies.

3. Transformation and Detection

DNA from the library described in paragraph 2 above was chilled on ice to which was added electrocompetent DH10B bacteria (Life Technologies, 20 ml). The bacteria and vector mixture was then electroporated as recommended by the manufacturer. Subsequently, SOC media (Life Technologies, 1 ml) was added and the mixture was incubated at 37° C. for 30 minutes. The transformants were then plated onto 20 standard 150 mm LB plates containing ampicillin and incubated for 16 hours (37° C.). Positive colonies were scraped off the plates and the DNA was isolated from the bacterial pellet using standard protocols, e.g. CsCl-gradient. The purified DNA was then carried on to the yeast protocols below.

The yeast methods were divided into three categories: (1) Transformation of yeast with the plasmid/cDNA combined vector; (2) Detection and isolation of yeast clones secreting amylase; and (3) PCR amplification of the insert directly from the yeast colony and purification of the DNA for sequencing and further analysis.

The yeast strain used was HD56-5A (ATCC-90785). This strain has the following genotype: MAT alpha, ura3-52, leu2-3, leu2-112, his3-11, his3-15, MAL⁺, SUC⁺, GAL⁺. Preferably, yeast mutants can be employed that have deficient post-translational pathways. Such mutants may have translocation deficient alleles in sec71, sec72, sec62, with truncated sec71 being most preferred. Alternatively, antagonists (including antisense nucleotides and/or ligands) which interfere with the normal operation of these genes, other proteins implicated in this post translation pathway (e.g., SEC61p, SEC72p, SEC62p, SEC63p, TDJ1p or SSA1p-4p) or the complex formation of these proteins may also be preferably employed in combination with the amylase-expressing yeast.

Transformation was performed based on the protocol outlined by Gietz et al., Nucl. Acid. Res., 20:1425 (1992). Transformed cells were then inoculated from agar into YEPD complex media broth (100 ml) and grown overnight at 30° C. The YEPD broth was prepared as described in Kaiser et al., Methods in Yeast Genetics, Cold Spring Harbor Press, Cold Spring Harbor, N.Y., p. 207 (1994). The overnight culture was then diluted to about 2×10⁶ cells/ml (approx. OD₆₀₀=0.1) into fresh YEPD broth (500 ml) and regrown to 1×10⁷ cells/ml (approx. OD₆₀₀=0.4−0.5).

The cells were then harvested and prepared for transformation by transfer into GS3 rotor bottles in a Sorval GS3 rotor at 5,000 rpm for 5 minutes, the supernatant discarded, and then resuspended into sterile water, and centrifuged again in 50 ml falcon tubes at 3,500 rpm in a Beckman GS-6KR centrifuge. The supernatant was discarded and the cells were subsequently washed with LiAc/TE (10 ml, 10 mM Tris-HCl, 1 mM EDTA pH 7.5, 100 mM Li₂OOCCH₃), and resuspended into LiAc/TE (2.5 ml).

Transformation took place by mixing the prepared cells (100 μl) with freshly denatured single stranded salmon testes DNA (Lofstrand Labs, Gaithersburg, Md.) and transforming DNA (1 μg, vol.<10 μl) in microfuge tubes. The mixture was mixed briefly by vortexing, then 40% PEG/TE (600 μl, 40% polyethylene glycol-4000, 10 mM Tris-HCl, 1 mM EDTA, 100 mM Li₂OOCCH₃, pH 7.5) was added. This mixture was gently mixed and incubated at 30° C. while agitating for 30 minutes. The cells were then heat shocked at 42° C. for 15 minutes, and the reaction vessel centrifuged in a microfuge at 12,000 rpm for 5-10 seconds, decanted and resuspended into TE (500 μl, 10 mM Tris-HCl, 1 mM EDTA pH 7.5) followed by recentrifugation. The cells were then diluted into TE (1 ml) and aliquots (200 μl) were spread onto the selective media previously prepared in 150 mm growth plates (VWR).

Alternatively, instead of multiple small reactions, the transformation was performed using a single, large scale reaction, wherein reagent amounts were scaled up accordingly.

The selective media used was a synthetic complete dextrose agar lacking uracil (SCD-Ura) prepared as described in Kaiser et al., Methods in Yeast Genetics, Cold Spring Harbor Press, Cold Spring Harbor, N.Y., p. 208-210 (1994). Transformants were grown at 30° C. for 2-3 days.

The detection of colonies secreting amylase was performed by including red starch in the selective growth media. Starch was coupled to the red dye (Reactive Red-120, Sigma) as per the procedure described by Biely et al., Anal. Biochem., 172:176-179 (1988). The coupled starch was incorporated into the SCD-Ura agar plates at a final concentration of 0.15% (w/v), and was buffered with potassium phosphate to a pH of 7.0 (50-100 mM final concentration).

The positive colonies were picked and streaked across fresh selective media (onto 150 mm plates) in order to obtain well isolated and identifiable single colonies. Well isolated single colonies positive for amylase secretion were detected by direct incorporation of red starch into buffered SCD-Ura agar. Positive colonies were determined by their ability to break down starch resulting in a clear halo around the positive colony visualized directly.

4. Isolation of DNA by PCR Amplification

When a positive colony was isolated, a portion of it was picked by a toothpick and diluted into sterile water (30 μl) in a 96 well plate. At this time, the positive colonies were either frozen and stored for subsequent analysis or immediately amplified. An aliquot of cells (5 μl) was used as a template for the PCR reaction in a 25 μl volume containing: 0.5 μl Klentaq (Clontech, Palo Alto, Calif.); 4.0 μl 10 mM dNTP's (Perkin Elmer-Cetus); 2.5 μl Kentaq buffer (Clontech); 0.25 μl forward oligo 1; 0.25 μl reverse oligo 2; 12.5 μl distilled water. The sequence of the forward oligonucleotide 1 was:

5′-TGTAAAACGACGGCCAGTTAAATAGACCTGCAATTATTAATCT-3′ (SEQ ID NO: 611)

The sequence of reverse oligonucleotide 2 was:

5′-CAGGAAACAGCTATGACCACCTGCACACCTGCAAATCCATT-3′ (SEQ ID NO: 612)

PCR was then performed as follows:

| | | |
|---|---|---|
| a. | Denature | 92° C., 5 minutes |
| b. 3 cycles of: | Denature | 92° C., 30 seconds |
| | Anneal | 59° C., 30 seconds |
| | Extend | 72° C., 60 seconds |
| c. 3 cycles of: | Denature | 92° C., 30 seconds |
| | Anneal | 57° C., 30 seconds |
| | Extend | 72° C., 60 seconds |
| d. 25 cycles of: | Denature | 92° C., 30 seconds |
| | Anneal | 55° C., 30 seconds |
| | Extend | 72° C., 60 seconds |
| e. | Hold | 4° C. |

The underlined regions of the oligonucleotides annealed to the ADH promoter region and the amylase region, respectively, and amplified a 307 bp region from vector pSST-AMY.0 when no insert was present. Typically, the first 18 nucleotides of the 5′ end of these oligonucleotides contained annealing sites for the sequencing primers. Thus, the total product of the PCR reaction from an empty vector was 343 bp. However, signal sequence-fused cDNA resulted in considerably longer nucleotide sequences.

Following the PCR, an aliquot of the reaction (5 μl) was examined by agarose gel electrophoresis in a 1% agarose gel using a Tris-Borate-EDTA (TBE) buffering system as described by Sambrook et al., supra. Clones resulting in a single strong PCR product larger than 400 bp were further analyzed by DNA sequencing after purification with a 96 Qiaquick PCR clean-up column (Qiagen Inc., Chatsworth, Calif.).

### Example 3

### Isolation of cDNA Clones Using Signal Algorithm Analysis

Various polypeptide-encoding nucleic acid sequences were identified by applying a proprietary signal sequence finding algorithm developed by Genentech, Inc. (South San Francisco, Calif.) upon ESTs as well as clustered and assembled EST fragments from public (e.g., GenBank) and/or private (LIFESEQ®, Incyte Pharmaceuticals, Inc., Palo Alto, Calif.) databases. The signal sequence algorithm computes a secretion signal score based on the character of the DNA nucleotides surrounding the first and optionally the second methionine codon(s) (ATG) at the 5′-end of the sequence or sequence fragment under consideration. The nucleotides following the first ATG must code for at least 35 unambiguous amino acids without any stop codons. If the first ATG has the required amino acids, the second is not examined. If neither meets the requirement, the candidate sequence is not scored. In order to determine whether the EST sequence contains an authentic signal sequence, the DNA and corresponding amino acid sequences surrounding the ATG codon are scored using a set of seven sensors (evaluation parameters) known to be associated with secretion signals. Use of this algorithm resulted in the identification of numerous polypeptide-encoding nucleic acid sequences.

### Example 4

### Isolation of cDNA Clones Encoding Human PRO Polypeptides

Using the techniques described in Examples 1 to 3 above, numerous full-length cDNA clones were identified as encoding PRO polypeptides as disclosed herein. These cDNAs were then deposited under the terms of the Budapest Treaty with the American Type Culture Collection, 10801 University Blvd., Manassas, Va. 20110-2209, U.S.A. (ATCC) as shown in Table 7 below.

TABLE 7

| Material | ATCC Dep. No. | Deposit Date |
|---|---|---|
| DNA16435-1208 | 209930 | Jun. 2, 1998 |
| DNA23318-1211 | 209787 | Apr. 21, 1998 |
| DNA23322-1393 | 203400 | Oct. 27, 1998 |
| DNA23334-1392 | 209918 | Jun. 2, 1998 |
| DNA26843-1389 | 203099 | Aug. 4, 1998 |
| DNA26844-1394 | 209926 | Jun. 2, 1998 |
| DNA30867-1335 | 209807 | Apr. 28, 1998 |
| DNA33470-1175 | 209398 | Oct. 17, 1997 |
| DNA34436-1238 | 209523 | Dec. 10, 1997 |
| DNA35557-1137 | 209255 | Sep. 16, 1997 |
| DNA35599-1168 | 209373 | Oct. 16, 1997 |
| DNA35668-1171 | 209371 | Oct. 16, 1997 |
| DNA36992-1168 | 209382 | Oct. 16, 1997 |
| DNA39423-1182 | 209387 | Oct. 17, 1997 |
| DNA39427-1179 | 209395 | Oct. 17, 1997 |
| DNA39510-1181 | 209392 | Oct. 17, 1997 |
| DNA39518-1247 | 209529 | Dec. 10, 1997 |
| DNA39975-1210 | 209783 | Apr. 21, 1998 |
| DNA39976-1215 | 209524 | Dec. 10, 1997 |
| DNA39979-1213 | 209789 | Apr. 21, 1998 |
| DNA40594-1233 | 209617 | Feb. 5, 1998 |
| DNA40603-1232 | 209486 | Nov. 21, 1997 |
| DNA40604-1187 | 209394 | Oct. 17, 1997 |
| DNA40625-1189 | 209788 | Apr. 21, 1998 |
| DNA41225-1217 | 209491 | Nov. 21, 1997 |
| DNA41379-1236 | 209488 | Nov. 21, 1997 |
| DNA41386-1316 | 209703 | Mar. 26, 1998 |
| DNA44161-1434 | 209907 | May 27, 1998 |
| DNA44179-1362 | 209851 | May 6, 1998 |
| DNA44192-1246 | 209531 | Dec. 10, 1997 |
| DNA44694-1500 | 203114 | Aug. 11, 1998 |
| DNA45234-1277 | 209654 | Mar. 5, 1998 |
| DNA45409-2511 | 203579 | Jan. 12, 1999 |
| DNA45415-1318 | 209810 | Apr. 28, 1998 |
| DNA45417-1432 | 209910 | May 27, 1998 |
| DNA45493-1349 | 209805 | Apr. 28, 1998 |
| DNA46776-1284 | 209721 | Mar. 31, 1998 |
| DNA48296-1292 | 209668 | Mar. 11, 1998 |
| DNA48306-1291 | 209911 | May 27, 1998 |
| DNA48328-1355 | 209843 | May 6, 1998 |
| DNA48329-1290 | 209785 | Apr. 21, 1998 |
| DNA48334-1435 | 209924 | Jun. 2, 1998 |
| DNA49141-1431 | 203003 | Jun. 23, 1998 |
| DNA49624-1279 | 209655 | Mar. 5, 1998 |
| DNA49647-1398 | 209919 | Jun. 2, 1998 |
| DNA49819-1439 | 209931 | Jun. 2, 1998 |
| DNA50911-1288 | 209714 | Mar. 31, 1998 |
| DNA50914-1289 | 209722 | Mar. 31, 1998 |
| DNA50919-1361 | 209848 | May 6, 1998 |
| DNA50980-1286 | 209717 | Mar. 31, 1998 |
| DNA52185-1370 | 209861 | May 14, 1998 |
| DNA53906-1368 | 209747 | Apr. 7, 1998 |
| DNA53912-1457 | 209870 | May 14, 1998 |
| DNA53913-1490 | 203162 | Aug. 25, 1998 |
| DNA53977-1371 | 209862 | May 14, 1998 |
| DNA53978-1443 | 209983 | Jun. 16, 1998 |
| DNA53996-1442 | 209921 | Jun. 2, 1998 |
| DNA54002-1367 | 209754 | Apr. 7, 1998 |
| DNA55737-1345 | 209753 | Apr. 7, 1998 |
| DNA56050-1455 | 203011 | Jun. 23, 1998 |
| DNA56052-1454 | 203026 | Jun. 23, 1998 |
| DNA56107-1415 | 203405 | Oct. 27, 1998 |
| DNA56110-1437 | 203113 | Aug. 11, 1998 |
| DNA56406-1704 | 203478 | Nov. 17, 1998 |
| DNA56409-1377 | 209882 | May 20, 1998 |
| DNA56410-1414 | 209923 | Jun. 2, 1998 |
| DNA56436-1448 | 209902 | May 27, 1998 |
| DNA56529-1647 | 203293 | Sep. 29, 1998 |
| DNA56855-1447 | 203004 | Jun. 23, 1998 |
| DNA56859-1445 | 203019 | Jun. 23, 1998 |
| DNA56860-1510 | 209952 | Jun. 9, 1998 |
| DNA56865-1491 | 203022 | Jun. 23, 1998 |
| DNA56868-1478 | 203024 | Jun. 23, 1998 |
| DNA56869-1545 | 203161 | Aug. 25, 1998 |
| DNA56870-1492 | 209925 | Jun. 2, 1998 |
| DNA57039-1402 | 209777 | Apr. 14, 1998 |
| DNA57253-1382 | 209867 | May 14, 1998 |
| DNA57254-1477 | 203289 | Sep. 29, 1998 |
| DNA57699-1412 | 203020 | Jun. 23, 1998 |
| DNA57704-1452 | 209953 | Jun. 9, 1998 |
| DNA57710-1451 | 203048 | Jul. 1, 1998 |
| DNA57827-1493 | 203045 | Jul. 1, 1998 |
| DNA57844-1410 | 203010 | Jun. 23, 1998 |
| DNA58723-1588 | 203133 | Aug. 18, 1998 |
| DNA58727-1474 | 203171 | Sep. 1, 1998 |
| DNA58730-1607 | 203221 | Sep. 15, 1998 |
| DNA58732-1650 | 203290 | Sep. 29, 1998 |
| DNA58737-1473 | 203136 | Aug. 18, 1998 |
| DNA58743-1609 | 203154 | Aug. 25, 1998 |
| DNA58747-1384 | 209868 | May 14, 1998 |
| DNA58828-1519 | 203172 | Sep. 1, 1998 |
| DNA58846-1409 | 209957 | Jun. 9, 1998 |
| DNA58848-1472 | 209955 | Jun. 9, 1998 |
| DNA58849-1494 | 209958 | Jun. 9, 1998 |
| DNA58850-1495 | 209956 | Jun. 9, 1998 |
| DNA58852-1637 | 203271 | Sep. 22, 1998 |
| DNA58853-1423 | 203016 | Jun. 23, 1998 |
| DNA58855-1422 | 203018 | Jun. 23, 1998 |
| DNA59211-1450 | 209960 | Jun. 9, 1998 |
| DNA59212-1627 | 203245 | Sep. 9, 1998 |
| DNA59213-1487 | 209959 | Jun. 9, 1998 |
| DNA59219-1613 | 203220 | Sep. 15, 1998 |
| DNA59497-1496 | 209941 | Jun. 4, 1998 |
| DNA59602-1436 | 203051 | Jul. 1, 1998 |
| DNA59603-1419 | 209944 | Jun. 9, 1998 |
| DNA59605-1418 | 203005 | Jun. 23, 1998 |
| DNA59607-1497 | 209946 | Jun. 9, 1998 |
| DNA59610-1556 | 209990 | Jun. 16, 1998 |
| DNA59612-1466 | 209947 | Jun. 9, 1998 |
| DNA59613-1417 | 203007 | Jun. 23, 1998 |
| DNA59616-1465 | 209991 | Jun. 16, 1998 |
| DNA59619-1464 | 203041 | Jul. 1, 1998 |
| DNA59625-1498 | 209992 | Jun. 16, 1998 |
| DNA59817-1703 | 203470 | Nov. 17, 1998 |
| DNA59827-1426 | 203089 | Aug. 4, 1998 |
| DNA59828-1608 | 203158 | Aug. 25, 1998 |
| DNA59837-2545 | 203658 | Feb. 9, 1999 |
| DNA59844-2542 | 203650 | Feb. 9, 1999 |
| DNA59853-1505 | 209985 | Jun. 16, 1998 |
| DNA59854-1459 | 209974 | Jun. 16, 1998 |
| DNA59855-1485 | 209987 | Jun. 16, 1998 |
| DNA60278-1530 | 203170 | Sep. 1, 1998 |
| DNA60283-1484 | 203043 | Jul. 1, 1998 |
| DNA60608-1577 | 203126 | Aug. 18, 1998 |
| DNA60611-1524 | 203175 | Sep. 1, 1998 |
| DNA60619-1482 | 209993 | Jun. 16, 1998 |
| DNA60625-1507 | 209975 | Jun. 16, 1998 |
| DNA60629-1481 | 209979 | Jun. 16, 1998 |
| DNA60740-1615 | 203456 | Nov. 3, 1998 |
| DNA61608-1606 | 203239 | Sep. 9, 1998 |
| DNA61755-1554 | 203112 | Aug. 11, 1998 |
| DNA62809-1531 | 203237 | Sep. 9, 1998 |
| DNA62812-1594 | 203248 | Sep. 9, 1998 |
| DNA62813-2544 | 203655 | Feb. 9, 1999 |
| DNA62845-1684 | 203361 | Oct. 20, 1998 |
| DNA64849-1604 | 203468 | Nov. 17, 1998 |
| DNA64852-1589 | 203127 | Aug. 18, 1998 |
| DNA64863-1573 | 203251 | Sep. 9, 1998 |
| DNA64881-1602 | 203240 | Sep. 9, 1998 |
| DNA64902-1667 | 203317 | Oct. 6, 1998 |
| DNA64952-1568 | 203222 | Sep. 15, 1998 |
| DNA65403-1565 | 203230 | Sep. 15, 1998 |
| DNA65413-1534 | 203234 | Sep. 15, 1998 |
| DNA65423-1595 | 203227 | Sep. 15, 1998 |
| DNA66304-1546 | 203321 | Oct. 6, 1998 |
| DNA66308-1537 | 203159 | Aug. 25, 1998 |
| DNA66511-1563 | 203228 | Sep. 15, 1998 |
| DNA66512-1564 | 203218 | Sep. 15, 1998 |
| DNA66519-1535 | 203236 | Sep. 15, 1998 |
| DNA66521-1583 | 203225 | Sep. 15, 1998 |
| DNA66658-1584 | 203229 | Sep. 15, 1998 |
| DNA66660-1585 | 203279 | Sep. 22, 1998 |
| DNA66669-1597 | 203272 | Sep. 22, 1998 |
| DNA66674-1599 | 203281 | Sep. 22, 1998 |
| DNA68836-1656 | 203455 | Nov. 3, 1998 |
| DNA68862-2546 | 203652 | Feb. 9, 1999 |
| DNA68866-1644 | 203283 | Sep. 22, 1998 |
| DNA68869-1610 | 203164 | Aug. 25, 1998 |
| DNA68871-1638 | 203280 | Sep. 22, 1998 |
| DNA68879-1631 | 203274 | Sep. 22, 1998 |
| DNA68880-1676 | 203319 | Oct. 6, 1998 |
| DNA68882-1677 | 203318 | Oct. 6, 1998 |
| DNA68883-1691 | 203535 | Dec. 15, 1998 |
| DNA68885-1678 | 203311 | Oct. 6, 1998 |
| DNA71180-1655 | 203403 | Oct. 27, 1998 |
| DNA71184-1634 | 203266 | Sep. 22, 1998 |
| DNA71213-1659 | 203401 | Oct. 27, 1998 |
| DNA71234-1651 | 203402 | Oct. 27, 1998 |
| DNA71269-1621 | 203284 | Sep. 22, 1998 |
| DNA71277-1636 | 203285 | Sep. 22, 1998 |
| DNA71286-1687 | 203357 | Oct. 20, 1998 |
| DNA71883-1660 | 203475 | Nov. 17, 1998 |
| DNA73401-1633 | 203273 | Sep. 22, 1998 |
| DNA73492-1671 | 203324 | Oct. 6, 1998 |
| DNA73730-1679 | 203320 | Oct. 6, 1998 |
| DNA73734-1680 | 203363 | Oct. 20, 1998 |
| DNA73735-1681 | 203356 | Oct. 20, 1998 |
| DNA73742-1662 | 203316 | Oct. 6, 1998 |
| DNA73746-1654 | 203411 | Oct. 27, 1998 |
| DNA73760-1672 | 203314 | Oct. 6, 1998 |
| DNA76393-1664 | 203323 | Oct. 6, 1998 |
| DNA76398-1699 | 203474 | Nov. 17, 1998 |
| DNA76399-1700 | 203472 | Nov. 17, 1998 |
| DNA76522-2500 | 203469 | Nov. 17, 1998 |
| DNA76533-1689 | 203410 | Oct. 27, 1998 |
| DNA77303-2502 | 203479 | Nov. 17, 1998 |
| DNA77626-1705 | 203536 | Dec. 15, 1998 |
| DNA77648-1688 | 203408 | Oct. 27, 1998 |
| DNA81754-2532 | 203542 | Dec. 15, 1998 |
| DNA81757-2512 | 203543 | Dec. 15, 1998 |
| DNA82302-2529 | 203534 | Dec. 15, 1998 |
| DNA82340-2530 | 203547 | Dec. 22, 1998 |
| DNA87991-2540 | 203656 | Feb. 9, 1999 |
| DNA92238-2539 | 203602 | Jan. 20, 1999 |
| DNA115291-2681 | PTA-202 | Jun. 8, 1999 |
| DNA23336-2861 | PTA-1673 | Apr. 11, 2000 |
| DNA30862-1396 | 209920 | Jun. 2, 1998 |
| DNA30871-1157 | 209380 | Oct. 16, 1997 |
| DNA32279-1131 | 209259 | Sep. 16, 1997 |
| DNA33206-1165 | 209372 | Oct. 16, 1997 |
| DNA35673-1201 | 209418 | Oct. 28, 1997 |
| DNA47361-1154-2 | 209431 | Nov. 7, 1997 |
| DNA49631-1328 | 209806 | Apr. 28, 1998 |
| DNA52594-1270 | 209679 | Mar. 17, 1998 |
| DNA55800-1263 | 209680 | Mar. 17, 1998 |
| DNA56531-1648 | 203286 | Sep. 29, 1998 |
| DNA56965-1356 | 209842 | May 6, 1998 |
| DNA57037-1444 | 209903 | May 27, 1998 |
| DNA57695-1340 | 203006 | Jun. 23, 1998 |
| DNA57834-1339 | 209954 | Jun. 9, 1998 |
| DNA57841-1522 | 203458 | Nov. 3, 1998 |
| DNA58847-1383 | 209879 | May 20, 1998 |
| DNA59493-1420 | 203050 | Jul. 1, 1998 |
| DNA59586-1520 | 203288 | Sep. 29, 1998 |
| DNA59608-2577 | 203870 | Mar. 23, 1999 |
| DNA59849-1504 | 209986 | Jun. 16, 1998 |
| DNA60292-1506 | 203540 | Dec. 15, 1998 |
| DNA62377-1381-1 | 203552 | Dec. 22, 1998 |
| DNA62880-1513 | 203097 | Aug. 4, 1998 |
| DNA66672-1586 | 203265 | Sep. 22, 1998 |
| DNA67962-1649 | 203291 | Sep. 29, 1998 |
| DNA69555-2867 | PTA-1632 | Apr. 4, 2000 |
| DNA71162-2764 | PTA-860 | Oct. 19, 1999 |
| DNA71290-1630 | 203275 | Sep. 22, 1998 |
| DNA76401-1683 | 203360 | Oct. 20, 1998 |
| DNA76541-1675 | 203409 | Oct. 27, 1998 |
| DNA76788-2526 | 203551 | Dec. 22, 1998 |
| DNA77623-2524 | 203546 | Dec. 22, 1998 |
| DNA80136-2503 | 203541 | Dec. 15, 1998 |
| DNA83568-2692 | PTA-386 | Jul. 20, 1999 |
| DNA84210-2576 | 203818 | Mar. 2, 1999 |
| DNA86576-2595 | 203868 | Mar. 23, 1999 |
| DNA87976-2593 | 203888 | Mar. 30, 1999 |
| DNA92256-2596 | 203891 | Mar. 30, 1999 |
| DNA92289-2598 | PTA-131 | May 25, 1999 |
| DNA96850-2705 | PTA-479 | Aug. 3, 1999 |
| DNA96855-2629 | PTA-18 | May 4, 1999 |
| DNA96857-2636 | PTA-17 | May 4, 1999 |
| DNA96860-2700 | PTA-478 | Aug. 3, 1999 |
| DNA96861-2844 | PTA-1436 | Mar. 2, 2000 |
| DNA96866-2698 | PTA-491 | Aug. 3, 1999 |
| DNA96870-2676 | PTA-254 | Jun. 22, 1999 |
| DNA96872-2674 | PTA-550 | Aug. 17, 1999 |
| DNA96878-2626 | PTA-23 | May 4, 1999 |
| DNA96879-2619 | 203967 | Apr. 27, 1999 |
| DNA96889-2641 | PTA-119 | May 25, 1999 |
| DNA96893-2621 | PTA-12 | May 4, 1999 |
| DNA96897-2688 | PTA-379 | Jul. 20, 1999 |
| DNA98564-2643 | PTA-125 | May 25, 1999 |
| DNA107443-2718 | PTA-490 | Aug. 3, 1999 |
| DNA107786-2723 | PTA-474 | Aug. 3, 1999 |
| DNA108682-2712 | PTA-486 | Aug. 3, 1999 |
| DNA108684-2761 | PTA-653 | Sep. 14, 1999 |
| DNA108701-2749 | PTA-554 | Aug. 17, 1999 |
| DNA108720-2717 | PTA-511 | Aug. 10, 1999 |
| DNA108726-2729 | PTA-514 | Aug. 10, 1999 |
| DNA108728-2760 | PTA-654 | Sep. 14, 1999 |
| DNA108738-2767 | PTA-862 | Oct. 19, 1999 |
| DNA108743-2722 | PTA-508 | Aug. 10, 1999 |
| DNA108758-2759 | PTA-655 | Sep. 14, 1999 |
| DNA108765-2758 | PTA-657 | Sep. 14, 1999 |
| DNA108783-2747 | PTA-616 | Aug. 31, 1999 |
| DNA108789-2748 | PTA-547 | Aug. 17, 1999 |
| DNA108806-2724 | PTA-610 | Aug. 31, 1999 |
| DNA108936-2719 | PTA-519 | Aug. 10, 1999 |
| DNA119510-2771 | PTA-947 | Nov. 9, 1999 |
| DNA119517-2778 | PTA-951 | Nov. 16, 1999 |
| DNA119535-2756 | PTA-613 | Aug. 31, 1999 |
| DNA119537-2777 | PTA-956 | Nov. 16, 1999 |
| DNA119714-2851 | PTA-1537 | Mar. 21, 2000 |
| DNA125170-2780 | PTA-953 | Nov. 16, 1999 |
| DNA129594-2841 | PTA-1481 | Mar. 14, 2000 |
| DNA129793-2857 | PTA-1733 | Apr. 18, 2000 |
| DNA130809-2769 | PTA-949 | Nov. 9, 1999 |
| DNA131639-2874 | PTA-1784 | Apr. 25, 2000 |
| DNA131649-2855 | PTA-1482 | Mar. 14, 2000 |
| DNA131652-2876 | PTA-1628 | Apr. 4, 2000 |
| DNA131658-2875 | PTA-1671 | Apr. 11, 2000 |
| DNA132162-2770 | PTA-950 | Nov. 9, 1999 |
| DNA136110-2763 | PTA-652 | Sep. 14, 1999 |
| DNA139592-2866 | PTA-1587 | Mar. 28, 2000 |
| DNA139608-2856 | PTA-1581 | Mar. 28, 2000 |
| DNA143292-2848 | PTA-1778 | Apr. 25, 2000 |
| DNA144844-2843 | PTA-1536 | Mar. 21, 2000 |
| DNA144857-2845 | PTA-1589 | Mar. 28, 2000 |
| DNA145841-2868 | PTA-1678 | Apr. 11, 2000 |
| DNA148004-2882 | PTA-1779 | Apr. 25, 2000 |
| DNA149893-2873 | PTA-1672 | Apr. 11, 2000 |
| DNA149930-2884 | PTA-1668 | Apr. 11, 2000 |
| DNA150157-2898 | PTA-1777 | Apr. 25, 2000 |
| DNA150163-2842 | PTA-1533 | Mar. 21, 2000 |
| DNA153579-2894 | PTA-1729 | Apr. 18, 2000 |
| DNA164625-2890 | PTA-1535 | Mar. 21, 2000 |
| DNA57838-1337 | 203014 | Jun. 23, 1998 |
| DNA59777-1480 | 203111 | Aug. 11, 1998 |
| DNA66675-1587 | 203282 | Sep. 22, 1998 |
| DNA76532-1702 | 203473 | Nov. 17, 1998 |
| DNA105849-2704 | PTA-473 | Aug. 3, 1999 |
| DNA83500-2506 | 203391 | Oct. 29, 1998 |

These deposits were made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposits will be made available by ATCC under the terms of the Budapest Treaty, and subject to an agreement between Genentech, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC § 122 and the Commissioner's rules pursuant thereto (including 37 CFR § 1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

### Example 5

### Use of PRO as a Hybridization Probe

The following method describes use of a nucleotide sequence encoding PRO as a hybridization probe.

DNA comprising the coding sequence of full-length or mature PRO as disclosed herein is employed as a probe to screen for homologous DNAs (such as those encoding naturally-occurring variants of PRO) in human tissue cDNA libraries or human tissue genomic libraries.

Hybridization and washing of filters containing either library DNAs is performed under the following high stringency conditions. Hybridization of radiolabeled PRO-derived probe to the filters is performed in a solution of 50% formamide, 5× SSC, 0.1% SDS, 0.1% sodium pyrophosphate, 50 mM sodium phosphate, pH 6.8, 2× Denhardt's solution, and 10% dextran sulfate at 42° C. for 20 hours. Washing of the filters is performed in an aqueous solution of 0.1× SSC and 0.1% SDS at 42° C.

DNAs having a desired sequence identity with the DNA encoding full-length native sequence PRO can then be identified using standard techniques known in the art.

### Example 6

### Expression of PRO in E. coli

This example illustrates preparation of an unglycosylated form of PRO by recombinant expression in E. coli.

The DNA sequence encoding PRO is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the PRO coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized PRO protein can then be purified using a metal chelating column under conditions that allow tight binding of the protein.

PRO may be expressed in E. coli in a poly-His tagged form, using the following procedure. The DNA encoding PRO is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) lon galE rpoHts(htpRts) clpP(lacIq). Transformants are first grown in LB containing 50 mg/ml carbenicillin at 30° C. with shaking until an O.D.600 of 3-5 is reached. Cultures are then diluted 50-100 fold into CRAP media (prepared by mixing 3.57 g (NH₄)₂SO₄, 0.71 g sodium citrate.2H2O, 1.07 g KCl, 5.36 g Difco yeast extract, 5.36 g Sheffield hycase SF in 500 mL water, as well as 110 mM MPOS, pH 7.3, 0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.1M and 0.02 M, respectively, and the solution is stirred overnight at 4° C. This step results in a denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni-NTA metal chelate column equilibrated in the metal chelate column buffer. The column is washed with additional buffer containing 50 mM imidazole (Calbiochem, Utrol grade), pH 7.4. The protein is eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting the sample slowly into freshly prepared refolding buffer consisting of: 20 mM Tris, pH 8.6, 0.3 M NaCl, 2.5 M urea, 5 mM cysteine, 20 mM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros R1/H reversed phase column using a mobile buffer of 0.1% TFA with elution with a gradient of acetonitrile from 10 to 80%. Aliquots of fractions with A280 absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded PRO polypeptide are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

Many of the PRO polypeptides disclosed herein were successfully expressed as described above.

### Example 7

### Expression of PRO in Mammalian Cells

This example illustrates preparation of a potentially glycosylated form of PRO by recombinant expression in mammalian cells.

The vector, pRK5 (see EP 307,247, published Mar. 15, 1989), is employed as the expression vector. Optionally, the PRO DNA is ligated into pRK5 with selected restriction enzymes to allow insertion of the PRO DNA using ligation methods such as described in Sambrook et al., supra. The resulting vector is called pRK5-PRO.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-PRO DNA is mixed with about 1 μg DNA encoding the VA RNA gene [Thimmappaya et al., Cell, 31:543 (1982)] and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of PRO polypeptide. The cultures containing transfected cells may undergo further incubation (in serum free medium) and the medium is tested in selected bioassays.

In an alternative technique, PRO may be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-PRO DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed PRO can then be concentrated and purified by any selected method, such as dialysis and/or column chromatography.

In another embodiment, PRO can be expressed in CHO cells. The pRK5-PRO can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of PRO polypeptide, the culture medium may be replaced with serum free medium. Preferably, the cultures are incubated for about 6 days, and then the conditioned medium is harvested. The medium containing the expressed PRO can then be concentrated and purified by any selected method.

Epitope-tagged PRO may also be expressed in host CHO cells. The PRO may be subcloned out of the pRK5 vector. The subclone insert can undergo PCR to fuse in frame with a selected epitope tag such as a poly-his tag into a Baculovirus expression vector. The poly-his tagged PRO insert can then be subcloned into a SV40 driven vector containing a selection marker such as DHFR for selection of stable clones. Finally, the CHO cells can be transfected (as described above) with the SV40 driven vector. Labeling may be performed, as described above, to verify expression. The culture medium containing the expressed poly-His tagged PRO can then be concentrated and purified by any selected method, such as by Ni²⁺-chelate affinity chromatography.

PRO may also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used expression in CHO cells is as described in Lucas et al., Nucl. Acids Res. 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents Superfect® (Qiagen), Dosper® or Fugene® (Boehringer Mannheim). The cells are grown as described in Lucas et al., supra. Approximately 3×10⁷ cells are frozen in an ampule for further growth and production as described below.

The ampules containing the plasmid DNA are thawed by placement into water bath and mixed by vortexing. The contents are pipetted into a centrifuge tube containing 10 mLs of media and centrifuged at 1000 rpm for 5 minutes. The supernatant is aspirated and the cells are resuspended in 10 mL of selective media (0.2 μm filtered PS20 with 5% 0.2 μm diafiltered fetal bovine serum). The cells are then aliquoted into a 100 mL spinner containing 90 mL of selective media. After 1-2 days, the cells are transferred into a 250 mL spinner filled with 150 mL selective growth medium and incubated at 37° C. After another 2-3 days, 250 mL, 500 mL and 2000 mL spinners are seeded with 3×10⁵ cells/mL. The cell media is exchanged with fresh media by centrifugation and resuspension in production medium. Although any suitable CHO media may be employed, a production medium described in U.S. Pat. No. 5,122,469, issued Jun. 16, 1992 may actually be used. A 3L production spinner is seeded at 1.2×10⁶ cells/mL. On day 0, the cell number pH is determined. On day 1, the spinner is sampled and sparging with filtered air is commenced. On day 2, the spinner is sampled, the temperature shifted to 33° C., and 30 mL of 500 g/L glucose and 0.6 mL of 10% antifoam (e.g., 35% polydimethylsiloxane emulsion, Dow Corning 365 Medical Grade Emulsion) taken. Throughout the production, the pH is adjusted as necessary to keep it at around 7.2. After 10 days, or until the viability dropped below 70%, the cell culture is harvested by centrifugation and filtering through a 0.22 μm filter. The filtrate was either stored at 4° C. or immediately loaded onto columns for purification.

For the poly-His tagged constructs, the proteins are purified using a Ni-NTA column (Qiagen). Before purification, imidazole is added to the conditioned media to a concentration of 5 mM. The conditioned media is pumped onto a 6 ml Ni-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc-containing) constructs are purified from the conditioned media as follows. The conditioned medium is pumped onto a 5 ml Protein A column (Pharmacia) which had been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μL of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

Many of the PRO polypeptides disclosed herein were successfully expressed as described above.

### Example 8

### Expression of PRO in Yeast

The following method describes recombinant expression of PRO in yeast.

First, yeast expression vectors are constructed for intracellular production or secretion of PRO from the ADH2/GAPDH promoter. DNA encoding PRO and the promoter is inserted into suitable restriction enzyme sites in the selected plasmid to direct intracellular expression of PRO. For secretion, DNA encoding PRO can be cloned into the selected plasmid, together with DNA encoding the ADH2/GAPDH promoter, a native PRO signal peptide or other mammalian signal peptide, or, for example, a yeast alpha-factor or invertase secretory signal/leader sequence, and linker sequences (if needed) for expression of PRO.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant PRO can subsequently be isolated and purified by removing the yeast cells from the fermentation medium by centrifugation and then concentrating the medium using selected cartridge filters. The concentrate containing PRO may further be purified using selected column chromatography resins.

Many of the PRO polypeptides disclosed herein were successfully expressed as described above.

### Example 9

### Expression of PRO in Baculovirus-Infected Insect Cells

The following method describes recombinant expression of PRO in Baculovirus-infected insect cells.

The sequence coding for PRO is fused upstream of an epitope tag contained within a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the sequence encoding PRO or the desired portion of the coding sequence of PRO such as the sequence encoding the extracellular domain of a transmembrane protein or the sequence encoding the mature protein if the protein is extracellular is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by O'Reilley et al., Baculovirus expression vectors: A Laboratory Manual, Oxford: Oxford University Press (1994).

Expressed poly-his tagged PRO can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl₂; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or Western blot with Ni²⁺-NTA-conjugated to alkaline phosphatase (Qiagen). Fractions containing the eluted His₁₀-tagged PRO are pooled and dialyzed against loading buffer.

Alternatively, purification of the IgG tagged (or Fc tagged) PRO can be performed using known chromatography techniques, including for instance, Protein A or protein G column chromatography.

Many of the PRO polypeptides disclosed herein were successfully expressed as described above.

### Example 10

### Preparation of Antibodies that Bind PRO

This example illustrates preparation of monoclonal antibodies which can specifically bind PRO.

Techniques for producing the monoclonal antibodies are known in the art and are described, for instance, in Goding, supra. Immunogens that may be employed include purified PRO, fusion proteins containing PRO, and cells expressing recombinant PRO on the cell surface. Selection of the immunogen can be made by the skilled artisan without undue experimentation.

Mice, such as Balb/c, are immunized with the PRO immunogen emulsified in complete Freund's adjuvant and injected subcutaneously or intraperitoneally in an amount from 1-100 micrograms. Alternatively, the immunogen is emulsified in MPL-TDM adjuvant (Ribi Irnmunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-PRO antibodies.

After a suitable antibody titer has been detected, the animals “positive” for antibodies can be injected with a final intravenous injection of PRO. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against PRO. Determination of “positive” hybridoma cells secreting the desired monoclonal antibodies against PRO is within the skill in the art.

The positive hybridoma cells can be injected intraperitoneally into syngeneic Balb/c mice to produce ascites containing the anti-PRO monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 11

### Purification of PRO Polypeptides Using Specific Antibodies

Native or recombinant PRO polypeptides may be purified by a variety of standard techniques in the art of protein purification. For example, pro-PRO polypeptide, mature PRO polypeptide, or pre-PRO polypeptide is purified by immunoaffinity chromatography using antibodies specific for the PRO polypeptide of interest. In general, an immunoaffinity column is constructed by covalently coupling the anti-PRO polypeptide antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE™ (Pharmacia LKB Biotechnology). The antibody is coupled to the resin, the resin is blocked, and the derivative resin is washed according to the manufacturer's instructions.

Such an immunoaffinity column is utilized in the purification of PRO polypeptide by preparing a fraction from cells containing PRO polypeptide in a soluble form. This preparation is derived by solubilization of the whole cell or of a subcellular fraction obtained via differential centrifugation by the addition of detergent or by other methods well known in the art. Alternatively, soluble PRO polypeptide containing a signal sequence may be secreted in useful quantity into the medium in which the cells are grown.

A soluble PRO polypeptide-containing preparation is passed over the immunoaffinity column, and the column is washed under conditions that allow the preferential absorbance of PRO polypeptide (e.g., high ionic strength buffers in the presence of detergent). Then, the column is eluted under conditions that disrupt antibody/PRO polypeptide binding (e.g., a low pH buffer such as approximately pH 2-3, or a high concentration of a chaotrope such as urea or thiocyanate ion), and PRO polypeptide is collected.

### Example 12

### Drug Screening

This invention is particularly useful for screening compounds by using PRO polypeptides or binding fragment thereof in any of a variety of drug screening techniques. The PRO polypeptide or fragment employed in such a test may either be free in solution, affixed to a solid support, borne on a cell surface, or located intracellularly. One method of drug screening utilizes eukaryotic or prokaryotic host cells which are stably transformed with recombinant nucleic acids expressing the PRO polypeptide or fragment. Drugs are screened against such transformed cells in competitive binding assays. Such cells, either in viable or fixed form, can be used for standard binding assays. One may measure, for example, the formation of complexes between PRO polypeptide or a fragment and the agent being tested. Alternatively, one can examine the diminution in complex formation between the PRO polypeptide and its target cell or target receptors caused by the agent being tested.

Thus, the present invention provides methods of screening for drugs or any other agents which can affect a PRO polypeptide-associated disease or disorder. These methods comprise contacting such an agent with an PRO polypeptide or fragment thereof and assaying (I) for the presence of a complex between the agent and the PRO polypeptide or fragment, or (ii) for the presence of a complex between the PRO polypeptide or fragment and the cell, by methods well known in the art. In such competitive binding assays, the PRO polypeptide or fragment is typically labeled. After suitable incubation, free PRO polypeptide or fragment is separated from that present in bound form, and the amount of free or uncomplexed label is a measure of the ability of the particular agent to bind to PRO polypeptide or to interfere with the PRO polypeptide/cell complex.

Another technique for drug screening provides high throughput screening for compounds having suitable binding affinity to a polypeptide and is described in detail in WO 84/03564, published on Sep. 13, 1984. Briefly stated, large numbers of different small peptide test compounds are synthesized on a solid substrate, such as plastic pins or some other surface. As applied to a PRO polypeptide, the peptide test compounds are reacted with PRO polypeptide and washed. Bound PRO polypeptide is detected by methods well known in the art. Purified PRO polypeptide can also be coated directly onto plates for use in the aforementioned drug screening techniques. In addition, non-neutralizing antibodies can be used to capture the peptide and immobilize it on the solid support.

This invention also contemplates the use of competitive drug screening assays in which neutralizing antibodies capable of binding PRO polypeptide specifically compete with a test compound for binding to PRO polypeptide or fragments thereof. In this manner, the antibodies can be used to detect the presence of any peptide which shares one or more antigenic determinants with PRO polypeptide.

### Example 13

### Rational Drug Design

The goal of rational drug design is to produce structural analogs of biologically active polypeptide of interest (i.e., a PRO polypeptide) or of small molecules with which they interact, e.g., agonists, antagonists, or inhibitors. Any of these examples can be used to fashion drugs which are more active or stable forms of the PRO polypeptide or which enhance or interfere with the function of the PRO polypeptide in vivo (c.f., Hodgson, Bio/Technology, 9: 19-21 (1991)).

In one approach, the three-dimensional structure of the PRO polypeptide, or of an PRO polypeptide-inhibitor complex, is determined by x-ray crystallography, by computer modeling or, most typically, by a combination of the two approaches. Both the shape and charges of the PRO polypeptide must be ascertained to elucidate the structure and to determine active site(s) of the molecule. Less often, useful information regarding the structure of the PRO polypeptide may be gained by modeling based on the structure of homologous proteins. In both cases, relevant structural information is used to design analogous PRO polypeptide-like molecules or to identify efficient inhibitors. Useful examples of rational drug design may include molecules which have improved activity or stability as shown by Braxton and Wells, Biochemistry, 31:7796-7801 (1992) or which act as inhibitors, agonists, or antagonists of native peptides as shown by Athauda et al., J. Biochem., 113:742-746 (1993).

It is also possible to isolate a target-specific antibody, selected by functional assay, as described above, and then to solve its crystal structure. This approach, in principle, yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced peptides. The isolated peptides would then act as the pharmacore.

By virtue of the present invention, sufficient amounts of the PRO polypeptide may be made available to perform such analytical studies as X-ray crystallography. In addition, knowledge of the PRO polypeptide amino acid sequence provided herein will provide guidance to those employing computer modeling techniques in place of or in addition to x-ray crystallography.

### Example 14

### Identification of PRO Polypeptides that Stimulate TNF-α Release in Human Blood (Assay 128)

This assay shows that certain PRO polypeptides of the present invention act to stimulate the release of TNF-α in human blood. PRO polypeptides testing positive in this assay are useful for, among other things, research purposes where stimulation of the release of TNF-α would be desired and for the therapeutic treatment of conditions wherein enhanced TNF-α release would be beneficial. Specifically, 200 μl of human blood supplemented with 50 mM Hepes buffer (pH 7.2) is aliquoted per well in a 96 well test plate. To each well is then added 300 μl of either the test PRO polypeptide in 50 mM Hepes buffer (at various concentrations) or 50 mM Hepes buffer alone (negative control) and the plates are incubated at 37° C. for 6 hours. The samples are then centrifuged and 50 μl of plasma is collected from each well and tested for the presence of TNF-α by ELISA assay. A positive in the assay is a higher amount of TNF-α in the PRO polypeptide treated samples as compared to the negative control samples.

The following PRO polypeptides tested positive in this assay: PRO1079, PRO827, PRO791, PRO1131, PRO1316, PRO1183, PRO1343, PRO1760, PRO1567, and PRO4333.

### Example 15

### Promotion of Chondrocyte Redifferentiation (Assay 129)

This assay is designed to determine whether PRO polypeptides of the present invention show the ability to induce the proliferation and/or redifferentiation of chondrocytes in culture. PRO polypeptides testing positive in this assay would be expected to be useful for the therapeutic treatment of various bone and/or cartilage disorders such as, for example, sports injuries and arthritis.

Porcine chondrocytes are isolated by overnight collagenase digestion of articular cartilage of the metacarpophalangeal joint of 4-6 month old female pigs. The isolated cells are then seeded at 25,000 cells/cm² in Ham F-12 containing 10% FBS and 4 μg/ml gentamycin. The culture media is changed every third day. On day 12, the cells are seeded in 96 well plates at 5,000 cells/well in 100 μl of the same media without serum and 100 μl of either serum-free medium (negative control), staurosporin (final concentration of 5 nM; positive control) or the test PRO polypeptide are added to give a final volume of 200 μl/well. After 5 days at 37° C., 22 μl of media containing 100 μg/ml Hoechst 33342 and 50 μg/ml 5-CFDA is added to each well and incubated for an additional 10 minutes at 37° C. A picture of the green fluorescence is taken for each well and the differentiation state of the chondrocytes is calculated by morphometric analysis. A positive result in the assay is obtained when the >50% of the PRO polypeptide treated cells are differentiated (compared to the background obtained by the negative control).

PRO6029 polypeptide tested positive in this assay.

### Example 16

### Microarray Analysis to Detect Overexpression of PRO Polypeptides in Cancerous Tumors

Nucleic acid microarrays, often containing thousands of gene sequences, are useful for identifying differentially expressed genes in diseased tissues as compared to their normal counterparts. Using nucleic acid microarrays, test and control mRNA samples from test and control tissue samples are reverse transcribed and labeled to generate cDNA probes. The cDNA probes are then hybridized to an array of nucleic acids immobilized on a solid support. The array is configured such that the sequence and position of each member of the array is known. For example, a selection of genes known to be expressed in certain disease states may be arrayed on a solid support. Hybridization of a labeled probe with a particular array member indicates that the sample from which the probe was derived expresses that gene. If the hybridization signal of a probe from a test (disease tissue) sample is greater than hybridization signal of a probe from a control (normal tissue) sample, the gene or genes overexpressed in the disease tissue are identified. The implication of this result is that an overexpressed protein in a diseased tissue is useful not only as a diagnostic marker for the presence of the disease condition, but also as a therapeutic target for treatment of the disease condition.

The methodology of hybridization of nucleic acids and microarray technology is well known in the art. In the present example, the specific preparation of nucleic acids for hybridization and probes, slides, and hybridization conditions are all detailed in U.S. Provisional Patent Application Serial No. 60/193,767, filed on Mar. 31, 2000 and which is herein incorporated by reference.

In the present example, cancerous tumors derived from various human tissues were studied for PRO polypeptide-encoding gene expression relative to non-cancerous human tissue in an attempt to identify those PRO polypeptides which are overexpressed in cancerous tumors. Two sets of experimental data were generated. In one set, cancerous human colon tumor tissue and matched non-cancerous human colon tumor tissue from the same patient (“matched colon control”) were obtained and analyzed for PRO polypeptide expression using the above described microarray technology. In the second set of data, cancerous human tumor tissue from any of a variety of different human tumors was obtained and compared to a “universal” epithelial control sample which was prepared by pooling non-cancerous human tissues of epithelial origin, including liver, kidney, and lung. mRNA isolated from the pooled tissues represents a mixture of expressed gene products from these different tissues. Microarray hybridization experiments using the pooled control samples generated a linear plot in a 2-color analysis. The slope of the line generated in a 2-color analysis was then used to normalize the ratios of (test:control detection) within each experiment. The normalized ratios from various experiments were then compared and used to identify clustering of gene expression. Thus, the pooled “universal control” sample not only allowed effective relative gene expression determinations in a simple 2-sample comparison, it also allowed multi-sample comparisons across several experiments.

In the present experiments, nucleic acid probes derived from the herein described PRO polypeptide-encoding nucleic acid sequences were used in the creation of the microarray and RNA from the tumor tissues listed above were used for the hybridization thereto. A value based upon the normalized ratio:experimental ratio was designated as a “cutoff ratio”. Only values that were above this cutoff ratio were determined to be significant. Table 8 below shows the results of these experiments, demonstrating that various PRO polypeptides of the present invention are significantly overexpressed in various human tumor tissues as compared to a non-cancerous human tissue control. As described above, these data demonstrate that the PRO polypeptides of the present invention are useful not only as diagnostic markers for the presence of one or more cancerous tumors, but also serve as therapeutic targets for the treatment of those tumors.

TABLE 8

| Molecule | is overexpressed in: | as compared to: |
|---|---|---|
| PRO276 | lung tumor | universal normal control |
| PRO284 | colon tumor | universal normal control |
| PRO284 | lung tumor | universal normal control |
| PRO284 | breast tumor | universal normal control |
| PRO193 | colon tumor | universal normal control |
| PRO193 | lung tumor | universal normal control |
| PRO193 | breast tumor | universal normal control |
| PRO193 | prostate tumor | universal normal control |
| PRO190 | colon tumor | universal normal control |
| PRO190 | lung tumor | universal normal control |
| PRO190 | breast tumor | universal normal control |
| PRO180 | colon tumor | universal normal control |
| PRO180 | lung tumor | universal normal control |
| PRO180 | breast tumor | universal normal control |
| PRO194 | colon tumor | universal normal control |
| PRO194 | lung tumor | universal normal control |
| PRO194 | breast tumor | universal normal control |
| PRO194 | cervical tumor | universal normal control |
| PRO218 | colon tumor | universal normal control |
| PRO218 | lung tumor | universal normal control |
| PRO260 | colon tumor | universal normal control |
| PRO260 | lung tumor | universal normal control |
| PRO260 | breast tumor | universal normal control |
| PRO260 | rectal tumor | universal normal control |
| PRO233 | colon tumor | universal normal control |
| PRO233 | lung tumor | universal normal control |
| PRO233 | breast tumor | universal normal control |
| PRO234 | colon tumor | universal normal control |
| PRO234 | lung tumor | universal normal control |
| PRO234 | breast tumor | universal normal control |
| PRO234 | liver tumor | universal normal control |
| PRO236 | colon tumor | universal normal control |
| PRO236 | lung tumor | universal normal control |
| PRO236 | breast tumor | universal normal control |
| PRO244 | colon tumor | universal normal control |
| PRO244 | lung tumor | universal normal control |
| PRO262 | colon tumor | universal normal control |
| PRO262 | lung tumor | universal normal control |
| PRO262 | breast tumor | universal normal control |
| PRO271 | colon tumor | universal normal control |
| PRO271 | lung tumor | universal normal control |
| PRO268 | colon tumor | universal normal control |
| PRO268 | lung tumor | universal normal control |
| PRO268 | breast tumor | universal normal control |
| PRO270 | colon tumor | universal normal control |
| PRO270 | lung tumor | universal normal control |
| PRO270 | breast tumor | universal normal control |
| PRO270 | liver tumor | universal normal control |
| PRO355 | lung tumor | universal normal control |
| PRO355 | breast tumor | universal normal control |
| PRO355 | prostate tumor | universal normal control |
| PRO298 | colon tumor | universal normal control |
| PRO298 | lung tumor | universal normal control |
| PRO298 | breast tumor | universal normal control |
| PRO299 | colon tumor | universal normal control |
| PRO299 | lung tumor | universal normal control |
| PRO299 | breast tumor | universal normal control |
| PRO296 | colon tumor | universal normal control |
| PRO296 | breast tumor | universal normal control |
| PRO329 | colon tumor | universal normal control |
| PRO329 | lung tumor | universal normal control |
| PRO329 | breast tumor | universal normal control |
| PRO330 | colon tumor | universal normal control |
| PRO330 | lung tumor | universal normal control |
| PRO294 | lung tumor | universal normal control |
| PRO294 | breast tumor | universal normal control |
| PRO300 | colon tumor | universal normal control |
| PRO300 | lung tumor | universal normal control |
| PRO300 | breast tumor | universal normal control |
| PRO307 | lung tumor | universal normal control |
| PRO334 | colon tumor | universal normal control |
| PRO334 | lung tumor | universal normal control |
| PRO334 | breast tumor | universal normal control |
| PRO334 | prostate tumor | universal normal control |
| PRO352 | colon tumor | universal normal control |
| PRO352 | lung tumor | universal normal control |
| PRO352 | breast tumor | universal normal control |
| PRO352 | liver tumor | universal normal control |
| PRO710 | breast tumor | universal normal control |
| PRO873 | colon tumor | universal normal control |
| PRO873 | lung tumor | universal normal control |
| PRO873 | breast tumor | universal normal control |
| PRO873 | prostate tumor | universal normal control |
| PRO354 | colon tumor | universal normal control |
| PRO354 | lung tumor | universal normal control |
| PRO354 | breast tumor | universal normal control |
| PRO1151 | lung tumor | universal normal control |
| PRO1151 | breast tumor | universal normal control |
| PRO382 | colon tumor | universal normal control |
| PRO382 | lung tumor | universal normal control |
| PRO382 | breast tumor | universal normal control |
| PRO1864 | lung tumor | universal normal control |
| PRO1864 | breast tumor | universal normal control |
| PRO1864 | liver tumor | universal normal control |
| PRO386 | colon tumor | universal normal control |
| PRO386 | lung tumor | universal normal control |
| PRO386 | prostate tumor | universal normal control |
| PRO541 | colon tumor | universal normal control |
| PRO541 | lung tumor | universal normal control |
| PRO541 | breast tumor | universal normal control |
| PRO852 | breast tumor | universal normal control |
| PRO700 | colon tumor | universal normal control |
| PRO700 | lung tumor | universal normal control |
| PRO700 | breast tumor | universal normal control |
| PRO700 | rectal tumor | universal normal control |
| PRO708 | colon tumor | universal normal control |
| PRO708 | lung tumor | universal normal control |
| PRO708 | breast tumor | universal normal control |
| PRO707 | colon tumor | universal normal control |
| PRO707 | lung tumor | universal normal control |
| PRO864 | colon tumor | universal normal control |
| PRO864 | lung tumor | universal normal control |
| PRO864 | breast tumor | universal normal control |
| PRO706 | colon tumor | universal normal control |
| PRO706 | lung tumor | universal normal control |
| PRO706 | breast tumor | universal normal control |
| PRO706 | liver tumor | universal normal control |
| PRO732 | lung tumor | universal normal control |
| PRO732 | breast tumor | universal normal control |
| PRO732 | cervical tumor | universal normal control |
| PRO537 | colon tumor | universal normal control |
| PRO537 | lung tumor | universal normal control |
| PRO537 | breast tumor | universal normal control |
| PRO545 | lung tumor | universal normal control |
| PRO545 | breast tumor | universal normal control |
| PRO718 | lung tumor | universal normal control |
| PRO718 | breast tumor | universal normal control |
| PRO872 | lung tumor | universal normal control |
| PRO872 | breast tumor | universal normal control |
| PRO872 | liver tumor | universal normal control |
| PRO704 | colon tumor | universal normal control |
| PRO704 | lung tumor | universal normal control |
| PRO704 | breast tumor | universal normal control |
| PRO705 | lung tumor | universal normal control |
| PRO705 | breast tumor | universal normal control |
| PRO871 | lung tumor | universal normal control |
| PRO871 | breast tumor | universal normal control |
| PRO871 | liver tumor | universal normal control |
| PRO702 | lung tumor | universal normal control |
| PRO944 | colon tumor | universal normal control |
| PRO944 | lung tumor | universal normal control |
| PRO944 | rectal tumor | universal normal control |
| PRO739 | lung tumor | universal normal control |
| PRO739 | breast tumor | universal normal control |
| PRO739 | prostate tumor | universal normal control |
| PRO941 | colon tumor | universal normal control |
| PRO941 | lung tumor | universal normal control |
| PRO941 | breast tumor | universal normal control |
| PRO941 | rectal tumor | universal normal control |
| PRO1082 | lung tumor | universal normal control |
| PRO1082 | breast tumor | universal normal control |
| PRO1133 | colon tumor | universal normal control |
| PRO1133 | lung tumor | universal normal control |
| PRO983 | colon tumor | universal normal control |
| PRO983 | lung tumor | universal normal control |
| PRO983 | breast tumor | universal normal control |
| PRO784 | colon tumor | universal normal control |
| PRO784 | lung tumor | universal normal control |
| PRO784 | breast tumor | universal normal control |
| PRO784 | prostate tumor | universal normal control |
| PRO783 | colon tumor | universal normal control |
| PRO783 | lung tumor | universal normal control |
| PRO783 | breast tumor | universal normal control |
| PRO783 | liver tumor | universal normal control |
| PRO940 | colon tumor | universal normal control |
| PRO940 | lung tumor | universal normal control |
| PRO940 | breast tumor | universal normal control |
| PRO768 | colon tumor | universal normal control |
| PRO768 | lung tumor | universal normal control |
| PRO768 | breast tumor | universal normal control |
| PRO1079 | colon tumor | universal normal control |
| PRO1079 | lung tumor | universal normal control |
| PRO1079 | breast tumor | universal normal control |
| PRO1079 | rectal tumor | universal normal control |
| PRO1078 | colon tumor | universal normal control |
| PRO1078 | lung tumor | universal normal control |
| PRO1018 | colon tumor | universal normal control |
| PRO1018 | lung tumor | universal normal control |
| PRO1018 | breast tumor | universal normal control |
| PRO793 | colon tumor | universal normal control |
| PRO793 | lung tumor | universal normal control |
| PRO793 | breast tumor | universal normal control |
| PRO793 | rectal tumor | universal normal control |
| PRO1773 | colon tumor | universal normal control |
| PRO1773 | lung tumor | universal normal control |
| PRO1773 | prostate tumor | universal normal control |
| PRO1014 | lung tumor | universal normal control |
| PRO1014 | breast tumor | universal normal control |
| PRO1013 | colon tumor | universal normal control |
| PRO1013 | lung tumor | universal normal control |
| PRO1013 | breast tumor | universal normal control |
| PRO1013 | liver tumor | universal normal control |
| PRO937 | colon tumor | universal normal control |
| PRO937 | lung tumor | universal normal control |
| PRO937 | breast tumor | universal normal control |
| PRO937 | cervical tumor | universal normal control |
| PRO937 | rectal tumor | universal normal control |
| PRO1477 | lung tumor | universal normal control |
| PRO1477 | breast tumor | universal normal control |
| PRO1477 | rectal tumor | universal normal control |
| PRO842 | colon tumor | universal normal control |
| PRO842 | lung tumor | universal normal control |
| PRO842 | breast tumor | universal normal control |
| PRO839 | colon tumor | universal normal control |
| PRO1180 | colon tumor | universal normal control |
| PRO1180 | lung tumor | universal normal control |
| PRO1180 | liver tumor | universal normal control |
| PRO1134 | lung tumor | universal normal control |
| PRO1134 | breast tumor | universal normal control |
| PRO1134 | prostate tumor | universal normal control |
| PRO1115 | colon tumor | universal normal control |
| PRO1115 | lung tumor | universal normal control |
| PRO1115 | breast tumor | universal normal control |
| PRO1277 | colon tumor | universal normal control |
| PRO1277 | lung tumor | universal normal control |
| PRO1135 | lung tumor | universal normal control |
| PRO1135 | breast tumor | universal normal control |
| PRO1135 | cervical tumor | universal normal control |
| PRO827 | colon tumor | universal normal control |
| PRO827 | lung tumor | universal normal control |
| PRO827 | prostate tumor | universal normal control |
| PRO827 | cervical tumor | universal normal control |
| PRO1057 | lung tumor | universal normal control |
| PRO1057 | breast tumor | universal normal control |
| PRO1113 | colon tumor | universal normal control |
| PRO1113 | lung tumor | universal normal control |
| PRO1006 | colon tumor | universal normal control |
| PRO1006 | lung tumor | universal normal control |
| PRO1006 | breast tumor | universal normal control |
| PRO1006 | rectal tumor | universal normal control |
| PRO1074 | lung tumor | universal normal control |
| PRO1074 | rectal tumor | universal normal control |
| PRO1073 | lung tumor | universal normal control |
| PRO1073 | breast tumor | universal normal control |
| PRO1136 | colon tumor | universal normal control |
| PRO1136 | lung tumor | universal normal control |
| PRO1136 | breast tumor | universal normal control |
| PRO1004 | lung tumor | universal normal control |
| PRO1344 | colon tumor | universal normal control |
| PRO1344 | lung tumor | universal normal control |
| PRO1344 | breast tumor | universal normal control |
| PRO1344 | rectal tumor | universal normal control |
| PRO1110 | colon tumor | universal normal control |
| PRO1110 | lung tumor | universal normal control |
| PRO1110 | breast tumor | universal normal control |
| PRO1378 | colon tumor | universal normal control |
| PRO1378 | lung tumor | universal normal control |
| PRO1378 | prostate tumor | universal normal control |
| PRO1378 | cervical tumor | universal normal control |
| PRO1481 | colon tumor | universal normal control |
| PRO1481 | lung tumor | universal normal control |
| PRO1109 | lung tumor | universal normal control |
| PRO1109 | breast tumor | universal normal control |
| PRO1383 | colon tumor | universal normal control |
| PRO1383 | lung tumor | universal normal control |
| PRO1383 | breast tumor | universal normal control |
| PRO1072 | lung tumor | universal normal control |
| PRO1189 | colon tumor | universal normal control |
| PRO1189 | lung tumor | universal normal control |
| PRO1189 | breast tumor | universal normal control |
| PRO1189 | prostate tumor | universal normal control |
| PRO1003 | colon tumor | universal normal control |
| PRO1003 | lung tumor | universal normal control |
| PRO1003 | breast tumor | universal normal control |
| PRO1003 | liver tumor | universal normal control |
| PRO1003 | rectal tumor | universal normal control |
| PRO1108 | colon tumor | universal normal control |
| PRO1108 | lung tumor | universal normal control |
| PRO1108 | breast tumor | universal normal control |
| PRO1137 | colon tumor | universal normal control |
| PRO1137 | lung tumor | universal normal control |
| PRO1137 | breast tumor | universal normal control |
| PRO1138 | colon tumor | universal normal control |
| PRO1138 | lung tumor | universal normal control |
| PRO1138 | breast tumor | universal normal control |
| PRO1415 | colon tumor | universal normal control |
| PRO1415 | lung tumor | universal normal control |
| PRO1415 | prostate tumor | universal normal control |
| PRO1054 | lung tumor | universal normal control |
| PRO1054 | breast tumor | universal normal control |
| PRO994 | colon tumor | universal normal control |
| PRO994 | lung tumor | universal normal control |
| PRO994 | rectal tumor | universal normal control |
| PRO1069 | lung tumor | universal normal control |
| PRO1069 | breast tumor | universal normal control |
| PRO1411 | colon tumor | universal normal control |
| PRO1411 | lung tumor | universal normal control |
| PRO1129 | lung tumor | universal normal control |
| PRO1129 | rectal tumor | universal normal control |
| PRO1359 | colon tumor | universal normal control |
| PRO1359 | lung tumor | universal normal control |
| PRO1359 | breast tumor | universal normal control |
| PRO1359 | prostate tumor | universal normal control |
| PRO1139 | lung tumor | universal normal control |
| PRO1065 | lung tumor | universal normal control |
| PRO1028 | colon tumor | universal normal control |
| PRO1028 | lung tumor | universal normal control |
| PRO1028 | breast tumor | universal normal control |
| PRO1028 | cervical tumor | universal normal control |
| PRO1027 | colon tumor | universal normal control |
| PRO1027 | lung tumor | universal normal control |
| PRO1027 | breast tumor | universal normal control |
| PRO1140 | colon tumor | universal normal control |
| PRO1140 | breast tumor | universal normal control |
| PRO1291 | colon tumor | universal normal control |
| PRO1291 | breast tumor | universal normal control |
| PRO1105 | colon tumor | universal normal control |
| PRO1105 | lung tumor | universal normal control |
| PRO1026 | lung tumor | universal normal control |
| PRO1026 | prostate tumor | universal normal control |
| PRO1104 | colon tumor | universal normal control |
| PRO1104 | lung tumor | universal normal control |
| PRO1104 | breast tumor | universal normal control |
| PRO1100 | colon tumor | universal normal control |
| PRO1100 | lung tumor | universal normal control |
| PRO1100 | breast tumor | universal normal control |
| PRO1100 | rectal tumor | universal normal control |
| PRO1141 | lung tumor | universal normal control |
| PRO1772 | colon tumor | universal normal control |
| PRO1772 | lung tumor | universal normal control |
| PRO1772 | breast tumor | universal normal control |
| PRO1772 | cervical tumor | universal normal control |
| PRO1064 | colon tumor | universal normal control |
| PRO1064 | lung tumor | universal normal control |
| PRO1379 | colon tumor | universal normal control |
| PRO1379 | lung tumor | universal normal control |
| PRO1379 | cervical tumor | universal normal control |
| PRO3573 | lung tumor | universal normal control |
| PRO3573 | breast tumor | universal normal control |
| PRO3566 | colon tumor | universal normal control |
| PRO3566 | lung tumor | universal normal control |
| PRO1156 | lung tumor | universal normal control |
| PRO1156 | breast tumor | universal normal control |
| PRO1156 | prostate tumor | universal normal control |
| PRO1098 | colon tumor | universal normal control |
| PRO1098 | lung tumor | universal normal control |
| PRO1098 | rectal tumor | universal normal control |
| PRO1128 | colon tumor | universal normal control |
| PRO1128 | lung tumor | universal normal control |
| PRO1128 | breast tumor | universal normal control |
| PRO1248 | lung tumor | universal normal control |
| PRO1248 | breast tumor | universal normal control |
| PRO1127 | colon tumor | universal normal control |
| PRO1127 | lung tumor | universal normal control |
| PRO1127 | breast tumor | universal normal control |
| PRO1316 | colon tumor | universal normal control |
| PRO1316 | lung tumor | universal normal control |
| PRO1316 | breast tumor | universal normal control |
| PRO1197 | colon tumor | universal normal control |
| PRO1197 | lung tumor | universal normal control |
| PRO1197 | breast tumor | universal normal control |
| PRO1125 | lung tumor | universal normal control |
| PRO1158 | breast tumor | universal normal control |
| PRO1124 | colon tumor | universal normal control |
| PRO1124 | lung tumor | universal normal control |
| PRO1380 | colon tumor | universal normal control |
| PRO1380 | lung tumor | universal normal control |
| PRO1380 | breast tumor | universal normal control |
| PRO1380 | liver tumor | universal normal control |
| PRO1377 | colon tumor | universal normal control |
| PRO1377 | lung tumor | universal normal control |
| PRO1287 | lung tumor | universal normal control |
| PRO1287 | breast tumor | universal normal control |
| PRO1249 | lung tumor | universal normal control |
| PRO1249 | breast tumor | universal normal control |
| PRO1335 | colon tumor | universal normal control |
| PRO1335 | lung tumor | universal normal control |
| PRO1335 | breast tumor | universal normal control |
| PRO3572 | lung tumor | universal normal control |
| PRO1599 | colon tumor | universal normal control |
| PRO1599 | lung tumor | universal normal control |
| PRO1599 | breast tumor | universal normal control |
| PRO1374 | lung tumor | universal normal control |
| PRO1374 | breast tumor | universal normal control |
| PRO1345 | lung tumor | universal normal control |
| PRO1345 | breast tumor | universal normal control |
| PRO1311 | lung tumor | universal normal control |
| PRO1311 | breast tumor | universal normal control |
| PRO1357 | colon tumor | universal normal control |
| PRO1357 | lung tumor | universal normal control |
| PRO1557 | colon tumor | universal normal control |
| PRO1557 | lung tumor | universal normal control |
| PRO1557 | breast tumor | universal normal control |
| PRO1305 | colon tumor | universal normal control |
| PRO1305 | lung tumor | universal normal control |
| PRO1305 | breast tumor | universal normal control |
| PRO1302 | colon tumor | universal normal control |
| PRO1302 | lung tumor | universal normal control |
| PRO1302 | breast tumor | universal normal control |
| PRO1302 | rectal tumor | universal normal control |
| PRO1266 | colon tumor | universal normal control |
| PRO1336 | colon tumor | universal normal control |
| PRO1336 | lung tumor | universal normal control |
| PRO1336 | breast tumor | universal normal control |
| PRO1278 | colon tumor | universal normal control |
| PRO1278 | lung tumor | universal normal control |
| PRO1270 | breast tumor | universal normal control |
| PRO1298 | colon tumor | universal normal control |
| PRO1298 | lung tumor | universal normal control |
| PRO1301 | lung tumor | universal normal control |
| PRO1301 | breast tumor | universal normal control |
| PRO1268 | colon tumor | universal normal control |
| PRO1268 | breast tumor | universal normal control |
| PRO1327 | lung tumor | universal normal control |
| PRO1327 | breast tumor | universal normal control |
| PRO1328 | colon tumor | universal normal control |
| PRO1328 | lung tumor | universal normal control |
| PRO1328 | breast tumor | universal normal control |
| PRO1329 | colon tumor | universal normal control |
| PRO1329 | lung tumor | universal normal control |
| PRO1329 | breast tumor | universal normal control |
| PRO1339 | colon tumor | universal normal control |
| PRO1339 | lung tumor | universal normal control |
| PRO1342 | colon tumor | universal normal control |
| PRO1342 | lung tumor | universal normal control |
| PRO1342 | breast tumor | universal normal control |
| PRO1342 | rectal tumor | universal normal control |
| PRO1487 | colon tumor | universal normal control |
| PRO1487 | breast tumor | universal normal control |
| PRO3579 | lung tumor | universal normal control |
| PRO3579 | breast tumor | universal normal control |
| PRO1472 | colon tumor | universal normal control |
| PRO1472 | lung tumor | universal normal control |
| PRO1385 | lung tumor | universal normal control |
| PRO1385 | breast tumor | universal normal control |
| PRO1461 | colon tumor | universal normal control |
| PRO1461 | lung tumor | universal normal control |
| PRO1461 | breast tumor | universal normal control |
| PRO1429 | colon tumor | universal normal control |
| PRO1429 | lung tumor | universal normal control |
| PRO1429 | breast tumor | universal normal control |
| PRO1568 | lung tumor | universal normal control |
| PRO1568 | breast tumor | universal normal control |
| PRO1569 | colon tumor | universal normal control |
| PRO1569 | lung tumor | universal normal control |
| PRO1569 | breast tumor | universal normal control |
| PRO1753 | colon tumor | universal normal control |
| PRO1753 | lung tumor | universal normal control |
| PRO1570 | colon tumor | universal normal control |
| PRO1570 | lung tumor | universal normal control |
| PRO1570 | breast tumor | universal normal control |
| PRO1570 | prostate tumor | universal normal control |
| PRO1570 | rectal tumor | universal normal control |
| PRO1559 | colon tumor | universal normal control |
| PRO1559 | lung tumor | universal normal control |
| PRO1559 | breast tumor | universal normal control |
| PRO1486 | lung tumor | universal normal control |
| PRO1486 | breast tumor | universal normal control |
| PRO1433 | colon tumor | universal normal control |
| PRO1433 | lung tumor | universal normal control |
| PRO1433 | breast tumor | universal normal control |
| PRO1433 | rectal tumor | universal normal control |
| PRO1490 | lung tumor | universal normal control |
| PRO1490 | breast tumor | universal normal control |
| PRO1482 | lung tumor | universal normal control |
| PRO1482 | breast tumor | universal normal control |
| PRO1409 | colon tumor | universal normal control |
| PRO1409 | lung tumor | universal normal control |
| PRO1409 | breast tumor | universal normal control |
| PRO1446 | colon tumor | universal normal control |
| PRO1446 | lung tumor | universal normal control |
| PRO1446 | breast tumor | universal normal control |
| PRO1446 | prostate tumor | universal normal control |
| PRO1604 | colon tumor | universal normal control |
| PRO1604 | lung tumor | universal normal control |
| PRO1604 | breast tumor | universal normal control |
| PRO1491 | colon tumor | universal normal control |
| PRO1491 | lung tumor | universal normal control |
| PRO1491 | breast tumor | universal normal control |
| PRO1431 | colon tumor | universal normal control |
| PRO1431 | lung tumor | universal normal control |
| PRO1563 | colon tumor | universal normal control |
| PRO1563 | lung tumor | universal normal control |
| PRO1563 | breast tumor | universal normal control |
| PRO1571 | colon tumor | universal normal control |
| PRO1571 | lung tumor | universal normal control |
| PRO1571 | breast tumor | universal normal control |
| PRO1572 | lung tumor | universal normal control |
| PRO1572 | prostate tumor | universal normal control |
| PRO1573 | lung tumor | universal normal control |
| PRO1573 | breast tumor | universal normal control |
| PRO1508 | lung tumor | universal normal control |
| PRO1508 | breast tumor | universal normal control |
| PRO1485 | colon tumor | universal normal control |
| PRO1485 | lung tumor | universal normal control |
| PRO1564 | colon tumor | universal normal control |
| PRO1564 | lung tumor | universal normal control |
| PRO1564 | breast tumor | universal normal control |
| PRO1550 | colon tumor | universal normal control |
| PRO1550 | lung tumor | universal normal control |
| PRO1550 | breast tumor | universal normal control |
| PRO1757 | lung tumor | universal normal control |
| PRO1757 | breast tumor | universal normal control |
| PRO1757 | prostate tumor | universal normal control |
| PRO1758 | lung tumor | universal normal control |
| PRO1781 | colon tumor | universal normal control |
| PRO1781 | lung tumor | universal normal control |
| PRO1781 | breast tumor | universal normal control |
| PRO1606 | lung tumor | universal normal control |
| PRO1606 | breast tumor | universal normal control |
| PRO1784 | colon tumor | universal normal control |
| PRO1784 | lung tumor | universal normal control |
| PRO1784 | breast tumor | universal normal control |
| PRO1774 | colon tumor | universal normal control |
| PRO1774 | lung tumor | universal normal control |
| PRO1774 | breast tumor | universal normal control |
| PRO1605 | colon tumor | universal normal control |
| PRO1605 | lung tumor | universal normal control |
| PRO1605 | prostate tumor | universal normal control |
| PRO1928 | colon tumor | universal normal control |
| PRO1928 | lung tumor | universal normal control |
| PRO1928 | cervical tumor | universal normal control |
| PRO1865 | lung tumor | universal normal control |
| PRO1865 | liver tumor | universal normal control |
| PRO1925 | lung tumor | universal normal control |
| PRO1926 | liver tumor | universal normal control |
| PRO2630 | colon tumor | universal normal control |
| PRO2630 | lung tumor | universal normal control |
| PRO2630 | breast tumor | universal normal control |
| PRO2630 | liver tumor | universal normal control |
| PRO3443 | colon tumor | universal normal control |
| PRO3443 | lung tumor | universal normal control |
| PRO3443 | breast tumor | universal normal control |
| PRO3301 | colon tumor | universal normal control |
| PRO3301 | lung tumor | universal normal control |
| PRO3301 | breast tumor | universal normal control |
| PRO3301 | rectal tumor | universal normal control |
| PRO3442 | colon tumor | universal normal control |
| PRO3442 | lung tumor | universal normal control |
| PRO3442 | rectal tumor | universal normal control |
| PRO4978 | colon tumor | universal normal control |
| PRO4978 | lung tumor | universal normal control |
| PRO4978 | breast tumor | universal normal control |
| PRO4978 | rectal tumor | universal normal control |
| PRO5801 | colon tumor | universal normal control |
| PRO5801 | breast tumor | universal normal control |
| PRO19630 | colon tumor | universal normal control |
| PRO203 | colon tumor | universal normal control |
| PRO204 | colon tumor | universal normal control |
| PRO204 | lung tumor | universal normal control |
| PRO204 | breast tumor | universal normal control |
| PRO204 | prostate tumor | universal normal control |
| PRO210 | colon tumor | universal normal control |
| PRO210 | lung tumor | universal normal control |
| PRO223 | lung tumor | universal normal control |
| PRO223 | breast tumor | universal normal control |
| PRO247 | colon tumor | universal normal control |
| PRO247 | lung tumor | universal normal control |
| PRO247 | breast | universal normal control |
| PRO358 | lung tumor | universal normal control |
| PRO358 | breast tumor | universal normal control |
| PRO358 | prostate tumor | universal normal control |
| PRO724 | lung tumor | universal normal control |
| PRO868 | colon tumor | universal normal control |
| PRO868 | lung tumor | universal normal control |
| PRO868 | prostate tumor | universal normal control |
| PRO868 | rectal tumor | universal normal control |
| PRO740 | colon tumor | universal normal control |
| PRO1478 | colon tumor | universal normal control |
| PRO1478 | lung tumor | universal normal control |
| PRO162 | colon tumor | universal normal control |
| PRO162 | lung tumor | universal normal control |
| PRO162 | breast tumor | universal normal control |
| PRO828 | colon tumor | universal normal control |
| PRO828 | lung tumor | universal normal control |
| PRO828 | breast tumor | universal normal control |
| PRO828 | cervical tumor | universal normal control |
| PRO828 | liver tumor | universal normal control |
| PRO819 | lung tumor | universal normal control |
| PRO819 | breast tumor | universal normal control |
| PRO819 | rectal tumor | universal normal control |
| PRO813 | colon tumor | universal normal control |
| PRO813 | lung tumor | universal normal control |
| PRO813 | breast tumor | universal normal control |
| PRO813 | prostate tumor | universal normal control |
| PRO1194 | colon tumor | universal normal control |
| PRO1194 | lung tumor | universal normal control |
| PRO1194 | breast tumor | universal normal control |
| PRO887 | colon tumor | universal normal control |
| PRO887 | lung tumor | universal normal control |
| PRO887 | rectal tumor | universal normal control |
| PRO1071 | colon tumor | universal normal control |
| PRO1071 | lung tumor | universal normal control |
| PRO1071 | breast tumor | universal normal control |
| PRO1029 | colon tumor | universal normal control |
| PRO1029 | lung tumor | universal normal control |
| PRO1029 | breast tumor | universal normal control |
| PRO1190 | lung tumor | universal normal control |
| PRO1190 | breast tumor | universal normal control |
| PRO4334 | lung tumor | universal normal control |
| PRO1155 | colon tumor | universal normal control |
| PRO1155 | lung tumor | universal normal control |
| PRO1157 | breast tumor | universal normal control |
| PRO1157 | cervical tumor | universal normal control |
| PRO1122 | lung tumor | universal normal control |
| PRO1122 | breast tumor | universal normal control |
| PRO1183 | colon tumor | universal normal control |
| PRO1183 | lung tumor | universal normal control |
| PRO1183 | breast tumor | universal normal control |
| PRO1337 | colon tumor | universal normal control |
| PRO1337 | lung tumor | universal normal control |
| PRO1337 | breast tumor | universal normal control |
| PRO1480 | colon tumor | universal normal control |
| PRO1480 | lung tumor | universal normal control |
| PRO1480 | breast tumor | universal normal control |
| PRO19645 | colon tumor | universal normal control |
| PRO9782 | colon tumor | universal normal control |
| PRO1419 | colon tumor | universal normal control |
| PRO1575 | colon tumor | universal normal control |
| PRO1575 | lung tumor | universal normal control |
| PRO1567 | colon tumor | universal normal control |
| PRO1567 | lung tumor | universal normal control |
| PRO1567 | breast tumor | universal normal control |
| PRO1891 | colon tumor | universal normal control |
| PRO1889 | colon tumor | universal normal control |
| PRO1889 | lung tumor | universal normal control |
| PRO1785 | lung tumor | universal normal control |
| PRO1785 | prostate tumor | universal normal control |
| PRO6003 | colon tumor | universal normal control |
| PRO4333 | colon tumor | universal normal control |
| PRO4356 | colon tumor | universal normal control |
| PRO4352 | colon tumor | universal normal control |
| PRO4354 | colon tumor | universal normal control |
| PRO4354 | lung tumor | universal normal control |
| PRO4354 | prostate tumor | universal normal control |
| PRO4369 | colon tumor | universal normal control |
| PRO6030 | colon tumor | universal normal control |
| PRO4433 | colon tumor | universal normal control |
| PRO4424 | colon tumor | universal normal control |
| PRO4424 | breast tumor | universal normal control |
| PRO6017 | colon tumor | universal normal control |
| PRO19563 | colon tumor | universal normal control |
| PRO6015 | colon tumor | universal normal control |
| PRO5779 | colon tumor | universal normal control |
| PRO5776 | colon tumor | universal normal control |
| PRO4430 | lung tumor | universal normal control |
| PRO4421 | colon tumor | universal normal control |
| PRO4499 | colon tumor | universal normal control |
| PRO4423 | colon tumor | universal normal control |
| PRO5998 | colon tumor | universal normal control |
| PRO5998 | lung tumor | universal normal control |
| PRO4501 | colon tumor | universal normal control |
| PRO6240 | colon tumor | universal normal control |
| PRO6245 | colon tumor | universal normal control |
| PRO6175 | colon tumor | universal normal control |
| PRO9742 | colon tumor | universal normal control |
| PRO7179 | colon tumor | universal normal control |
| PRO6239 | colon tumor | universal normal control |
| PRO6493 | colon tumor | universal normal control |
| PRO9741 | colon tumor | universal normal control |
| PRO9822 | colon tumor | universal normal control |
| PRO6244 | colon tumor | universal normal control |
| PRO9740 | colon tumor | universal normal control |
| PRO9739 | colon tumor | universal normal control |
| PRO7177 | colon tumor | universal normal control |
| PRO7178 | colon tumor | universal normal control |
| PRO6246 | colon tumor | universal normal control |
| PRO6241 | colon tumor | universal normal control |
| PRO9835 | colon tumor | universal normal control |
| PRO9857 | colon tumor | universal normal control |
| PRO7436 | colon tumor | universal normal control |
| PRO9856 | colon tumor | universal normal control |
| PRO19605 | colon tumor | universal normal control |
| PRO9859 | colon tumor | universal normal control |
| PRO12970 | colon tumor | universal normal control |
| PRO19626 | colon tumor | universal normal control |
| PRO9883 | colon tumor | universal normal control |
| PRO19670 | colon tumor | universal normal control |
| PRO19624 | colon tumor | universal normal control |
| PRO19680 | colon tumor | universal normal control |
| PRO19675 | colon tumor | universal normal control |
| PRO9834 | colon tumor | universal normal control |
| PRO9744 | colon tumor | universal normal control |
| PRO19644 | colon tumor | universal normal control |
| PRO19625 | colon tumor | universal normal control |
| PRO19597 | colon tumor | universal normal control |
| PRO16090 | colon tumor | universal normal control |
| PRO19576 | colon tumor | universal normal control |
| PRO19646 | colon tumor | universal normal control |
| PRO19814 | colon tumor | universal normal control |
| PRO19669 | colon tumor | universal normal control |
| PRO19818 | colon tumor | universal normal control |
| PRO20088 | colon tumor | universal normal control |
| PRO16089 | colon tumor | universal normal control |
| PRO20025 | colon tumor | universal normal control |
| PRO20040 | colon tumor | universal normal control |
| PRO1760 | adrenal tumor | universal normal control |
| PRO1760 | breast tumor | universal normal control |
| PRO1760 | cervical tumor | universal normal control |
| PRO1760 | colon tumor | universal normal control |
| PRO1760 | liver tumor | universal normal control |
| PRO1760 | lung tumor | universal normal control |
| PRO1760 | prostate tumor | universal normal control |
| PRO1760 | rectal tumor | universal normal control |
| PRO6029 | adrenal tumor | universal normal control |
| PRO6029 | colon tumor | universal normal control |
| PRO6029 | prostate tumor | universal normal control |
| PRO1801 | colon tumor | universal normal control |
| PRO1801 | lung tumor | universal normal control |

What is claimed is:

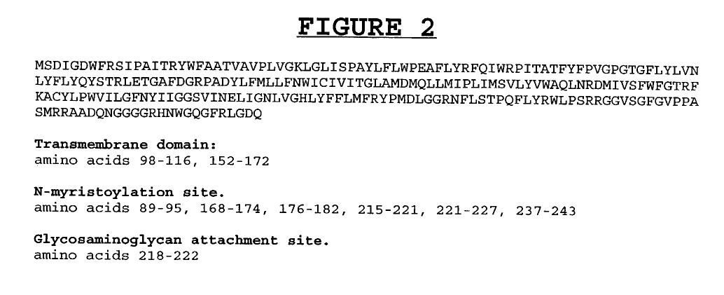

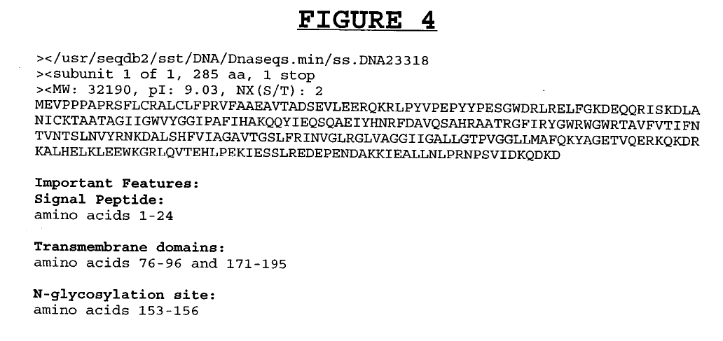

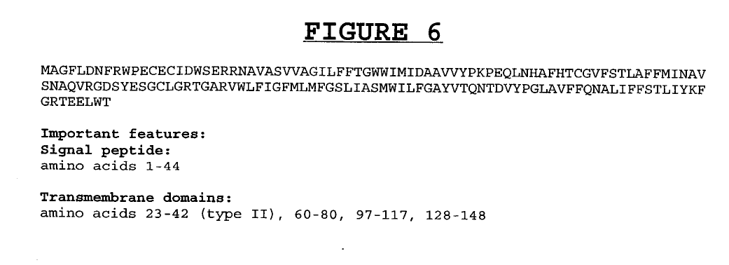

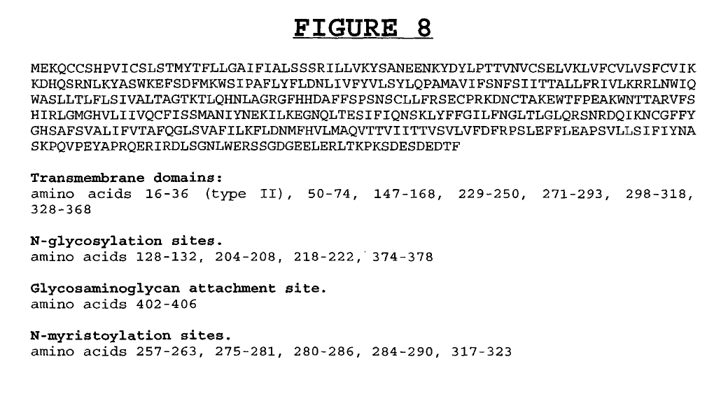

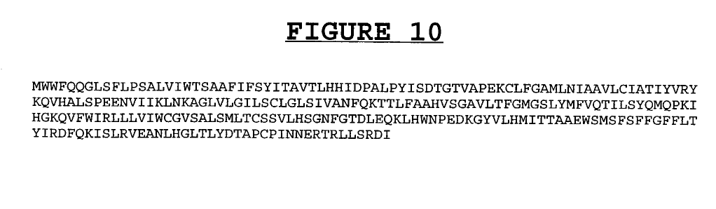

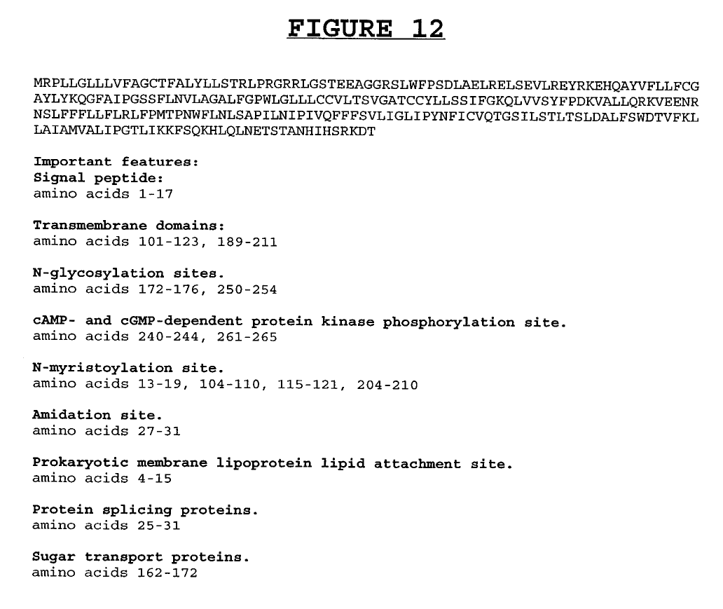

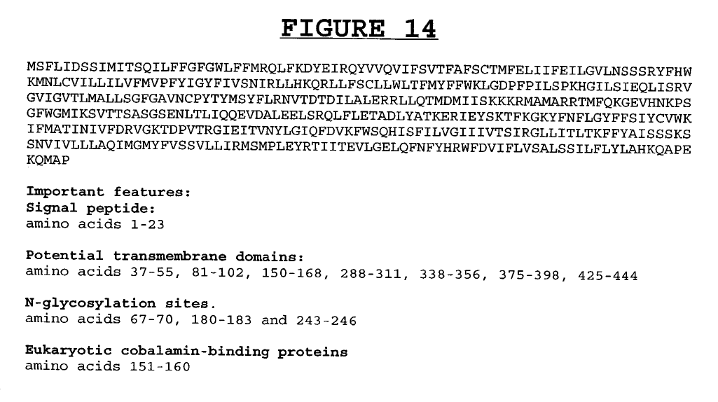

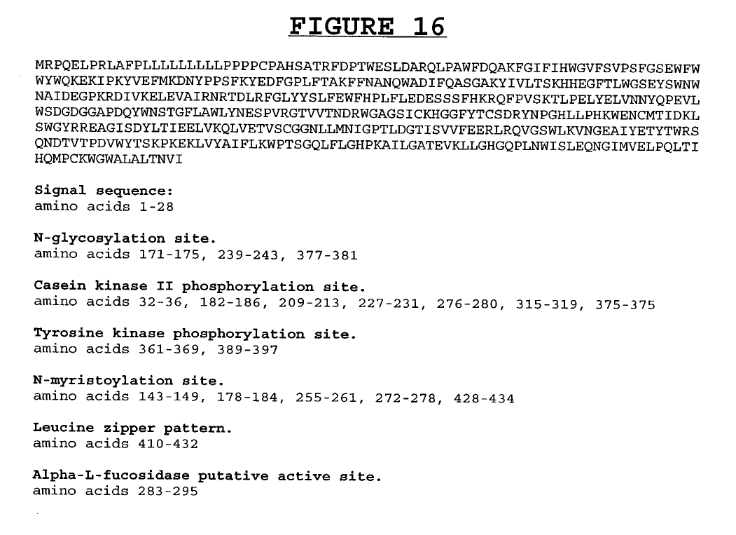

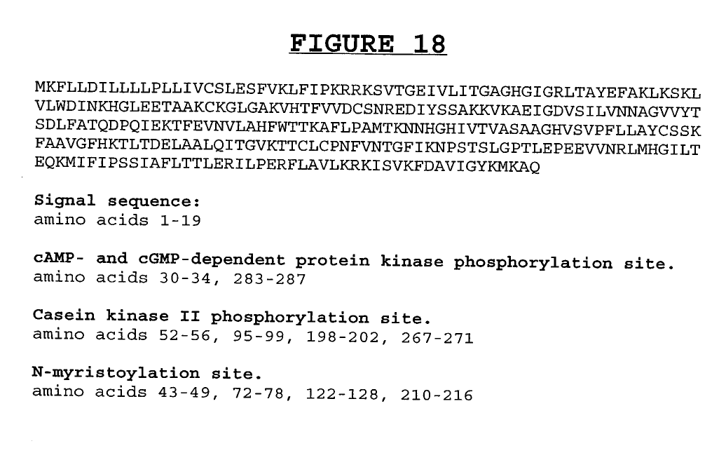

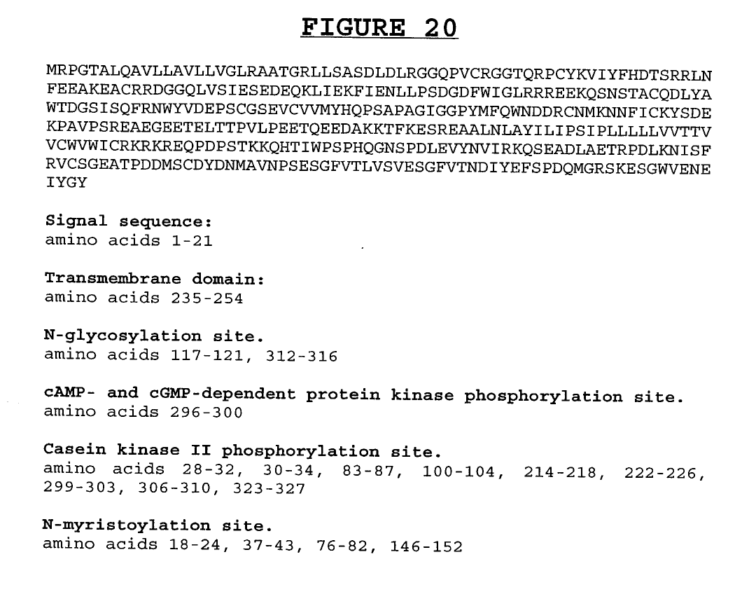

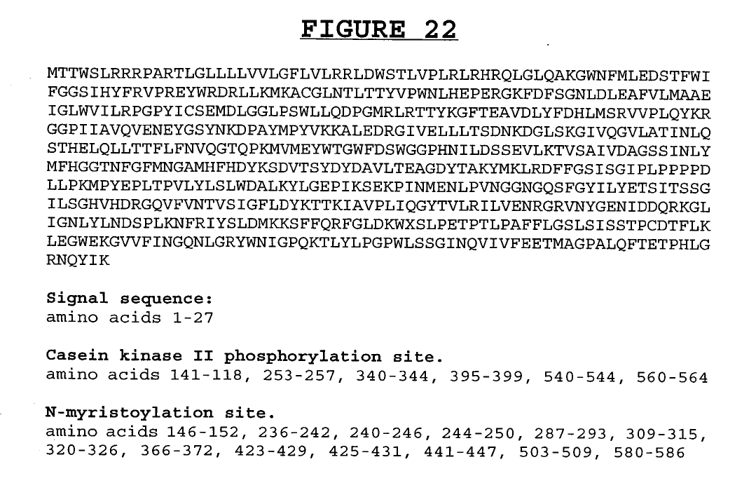

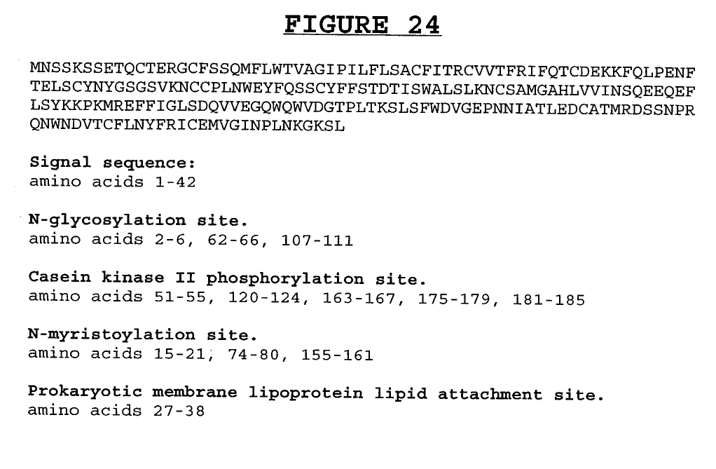

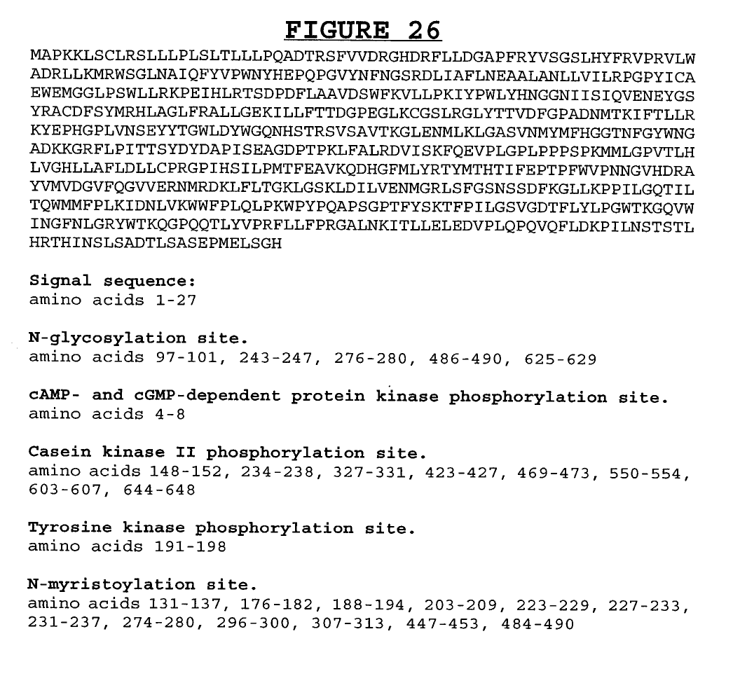

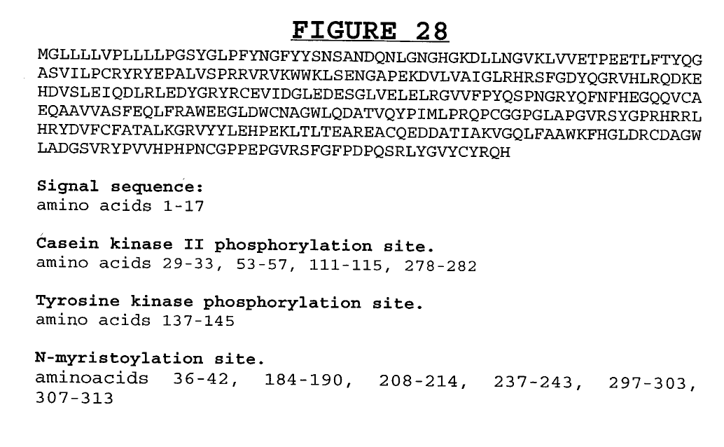

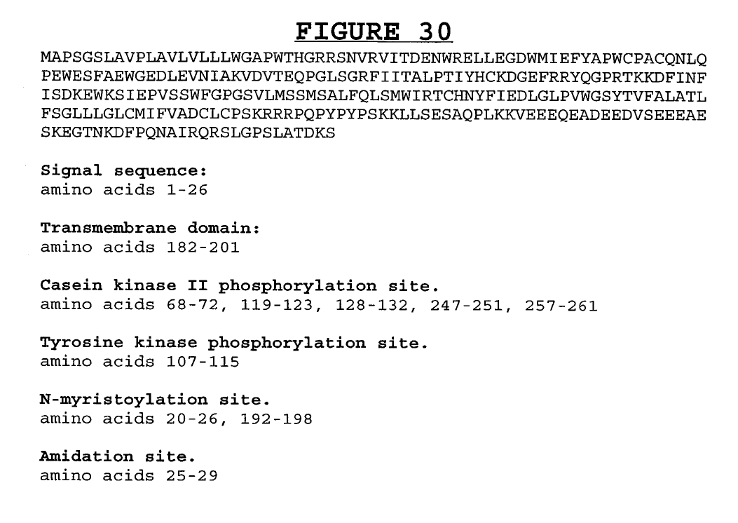

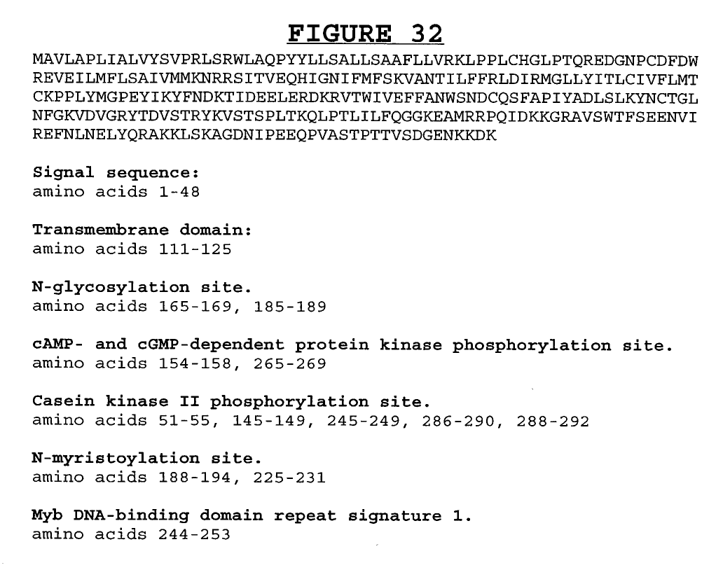

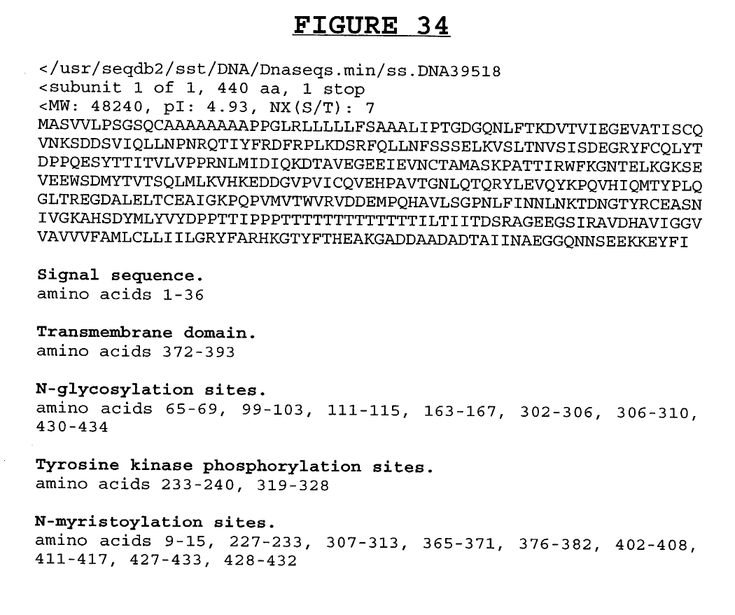

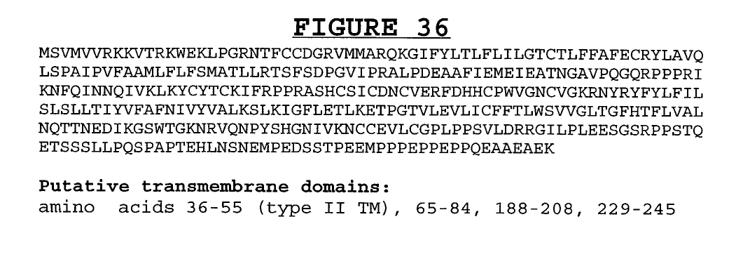

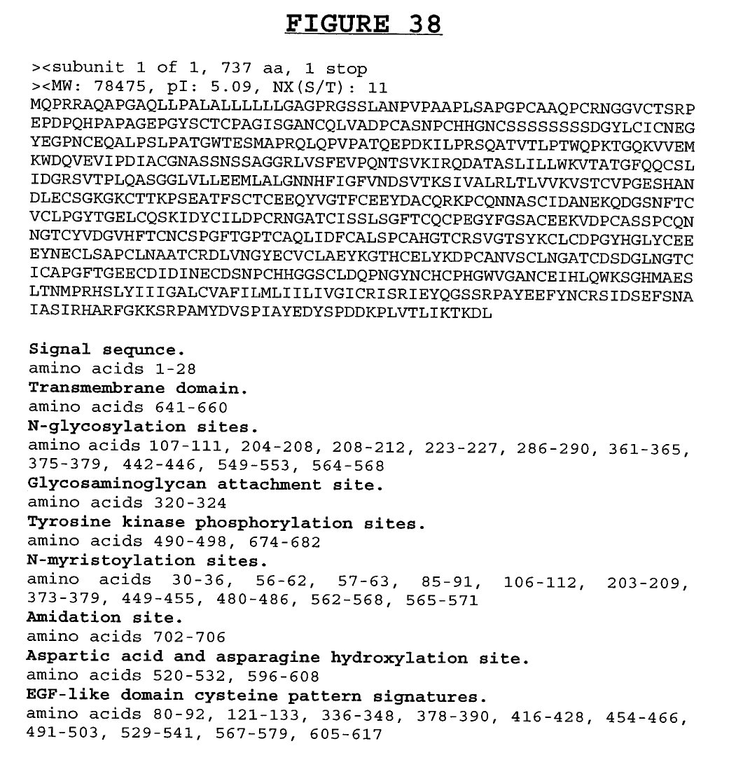

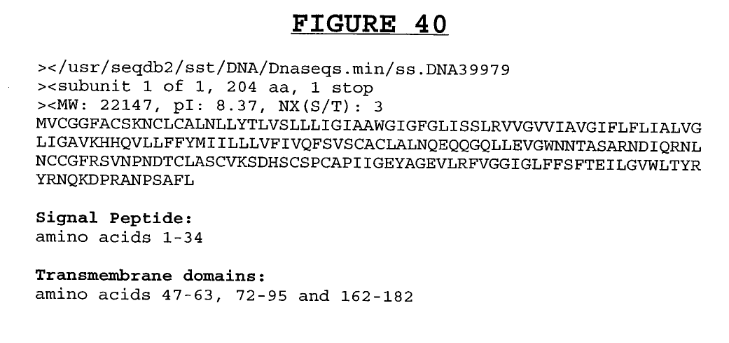

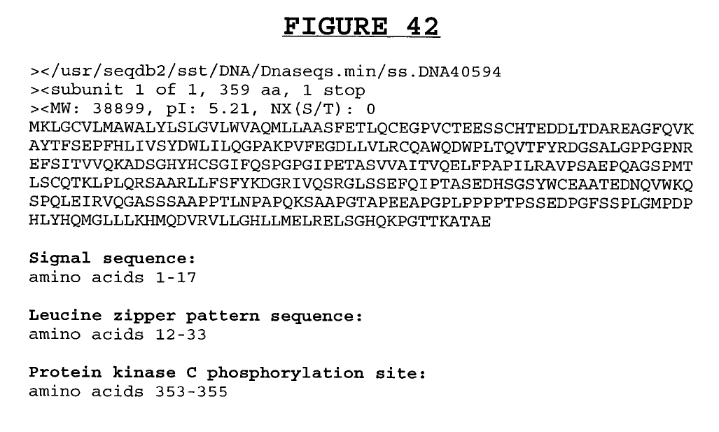

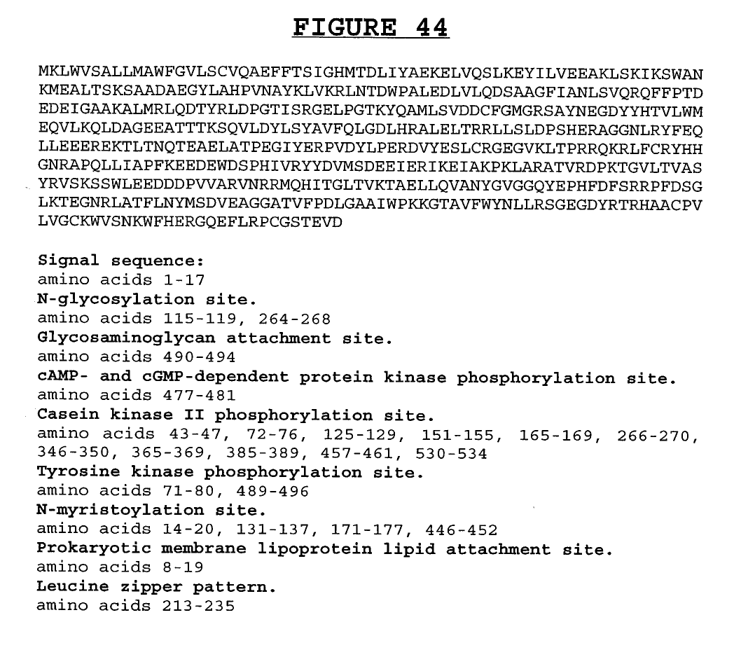

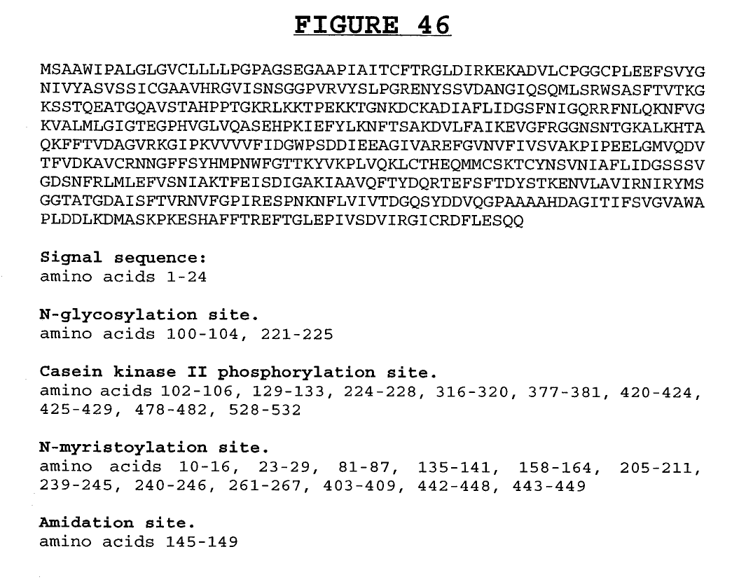

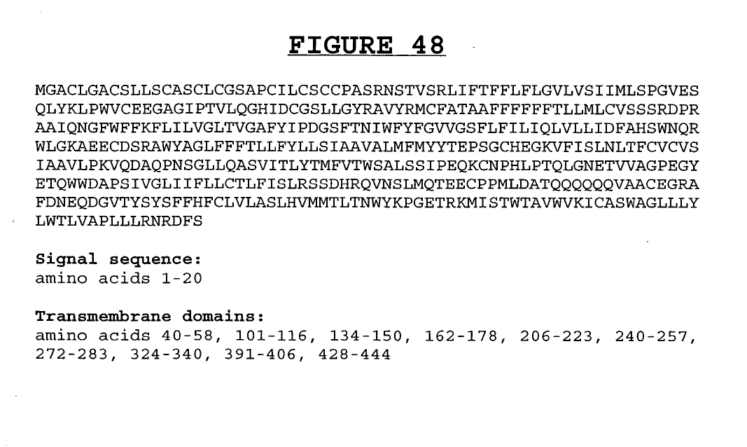

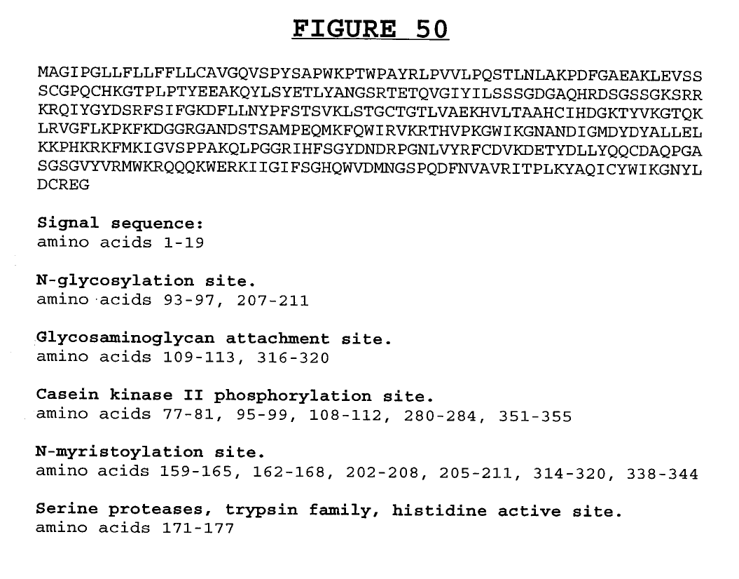

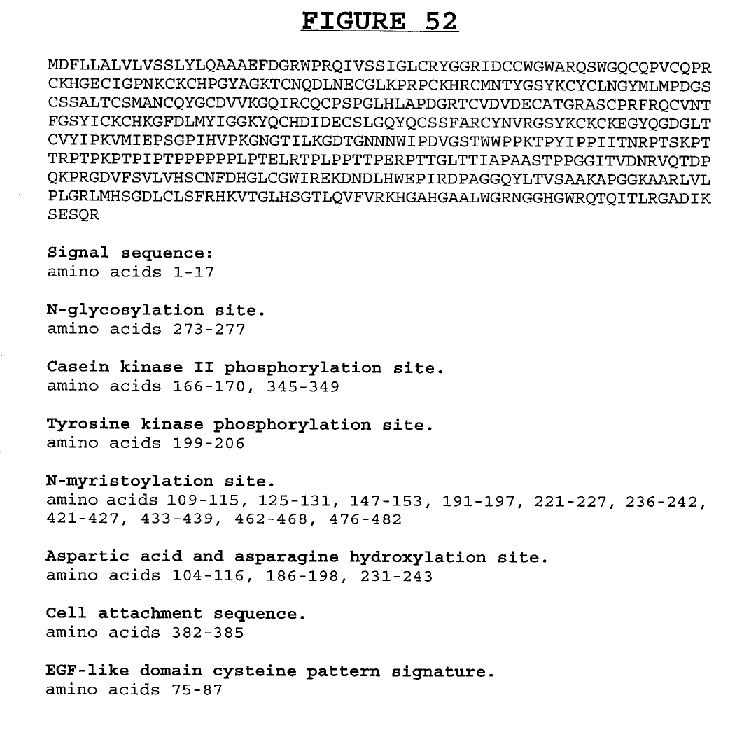

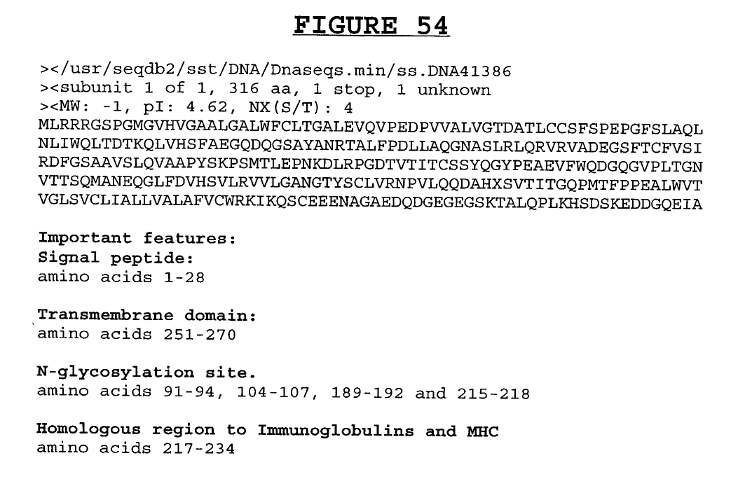

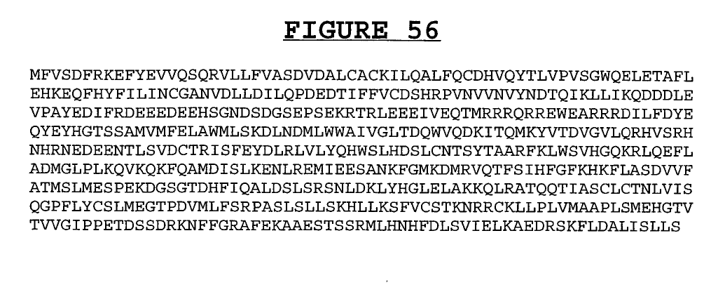

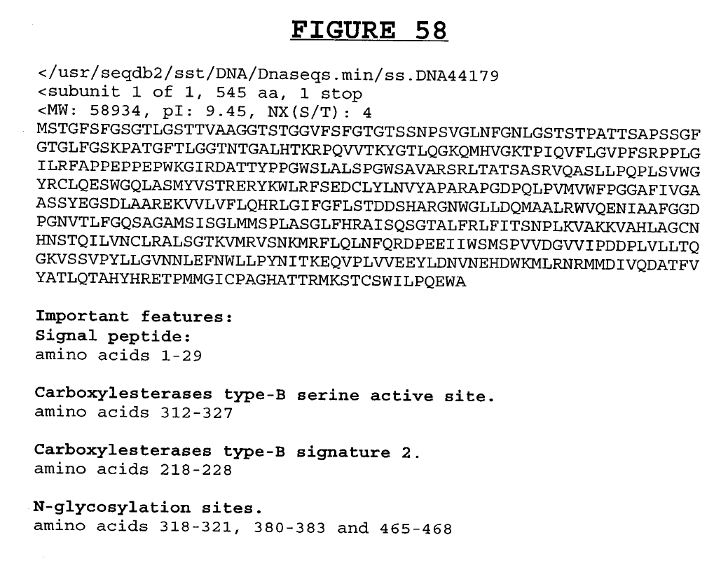

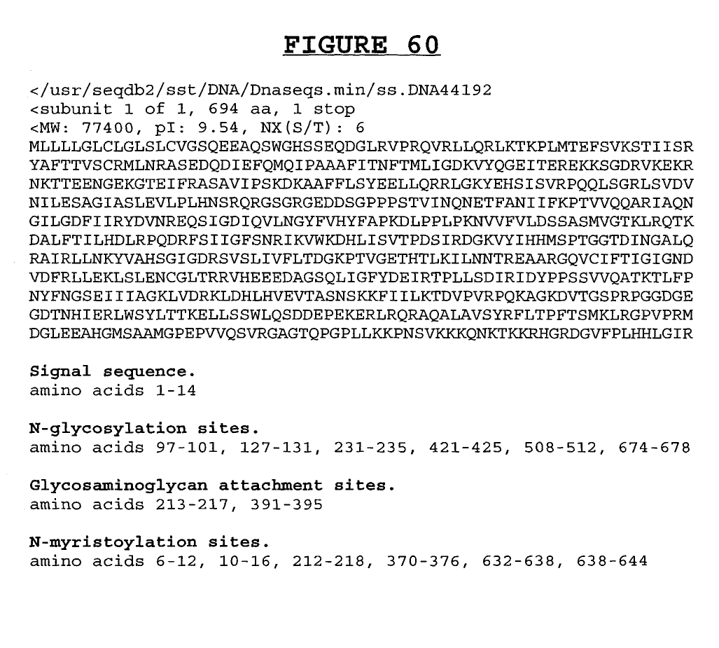

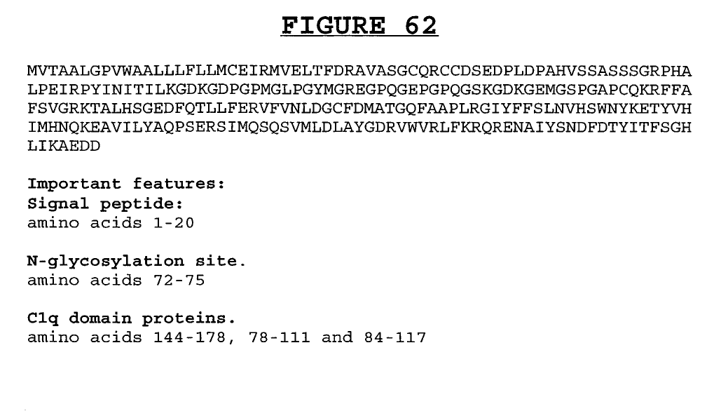

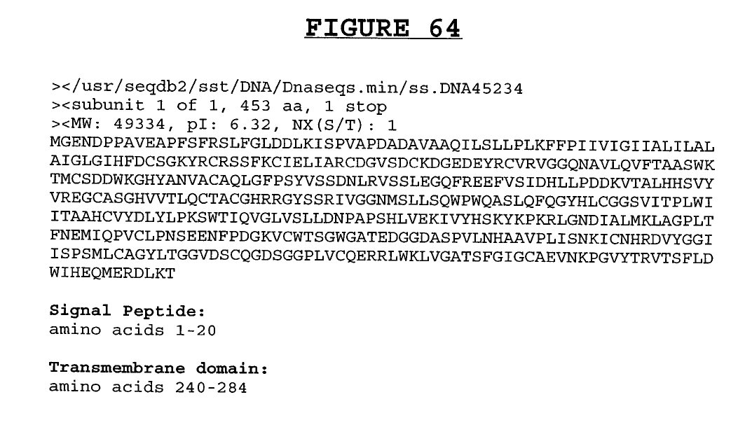

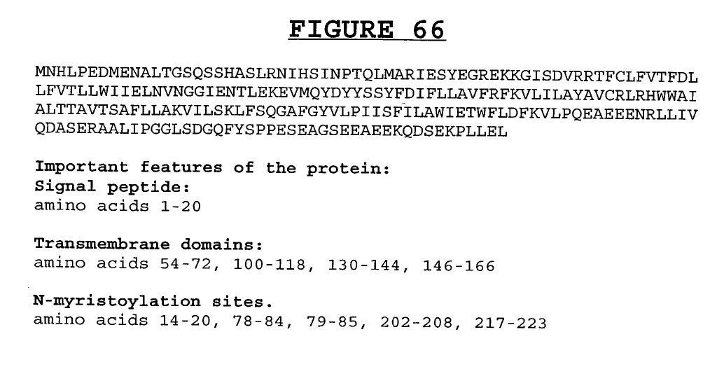

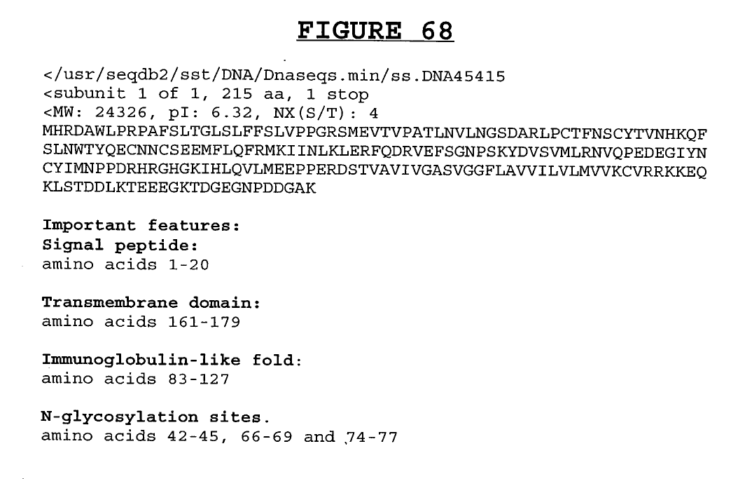

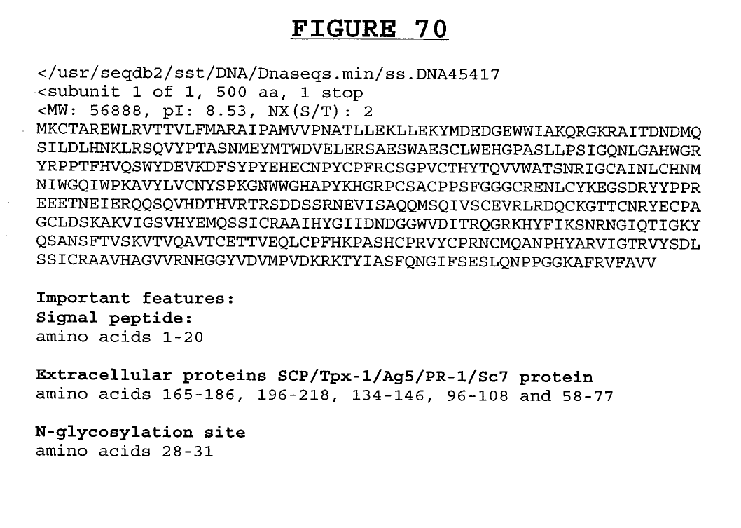

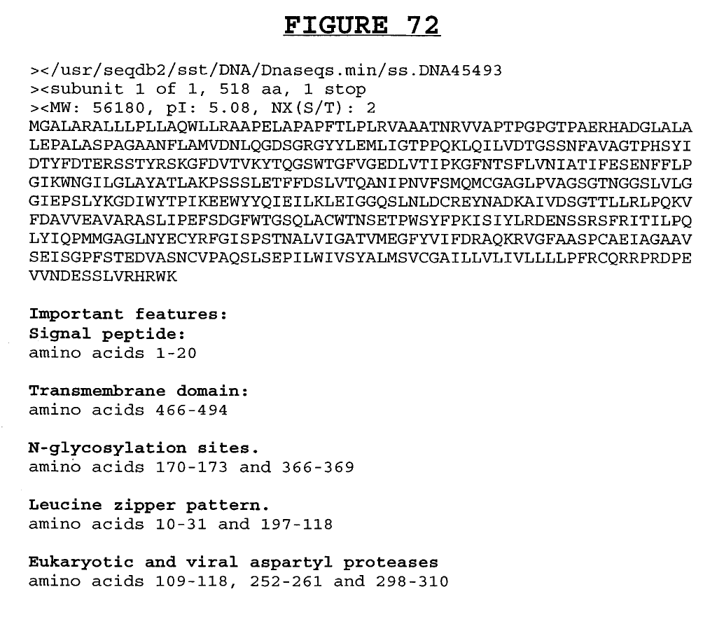

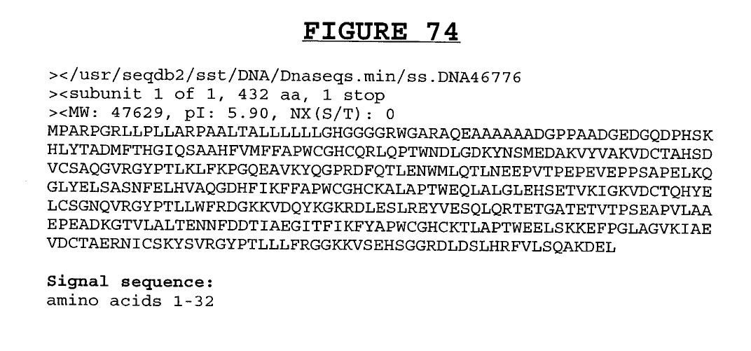

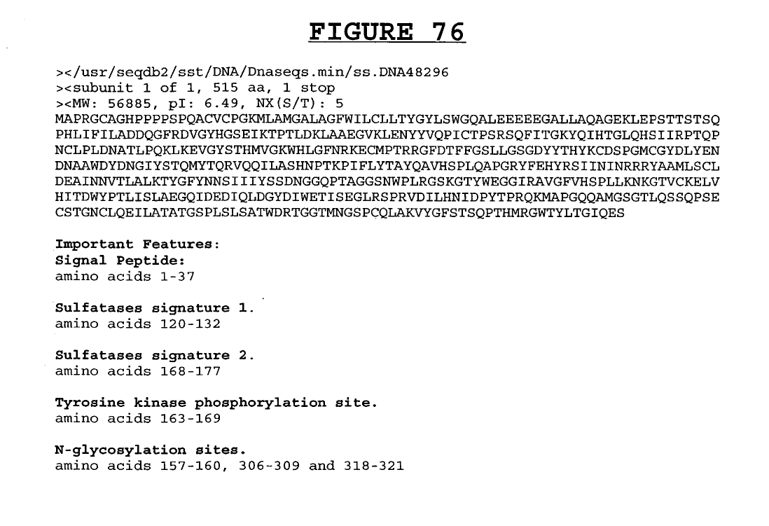

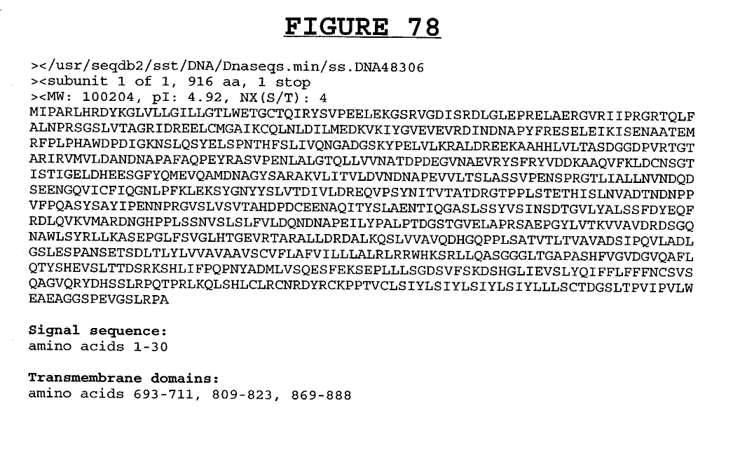

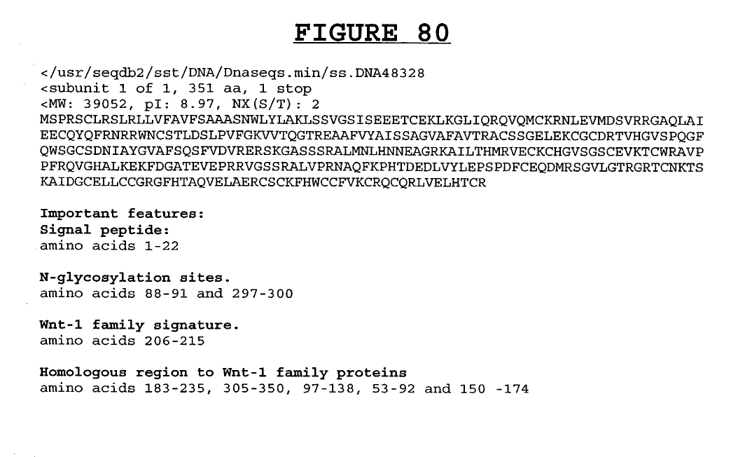

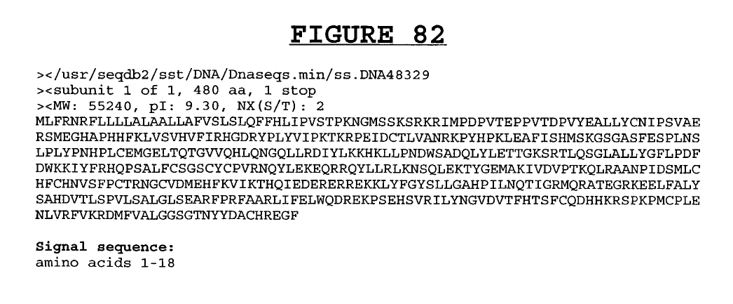

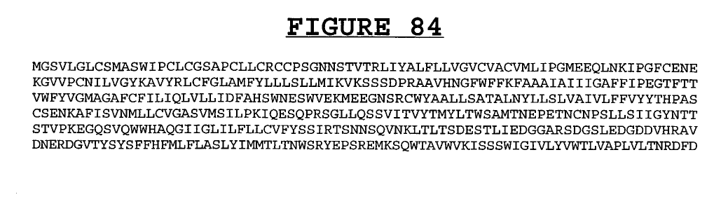

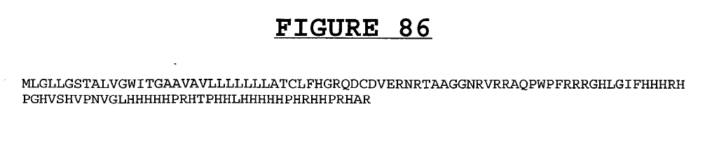

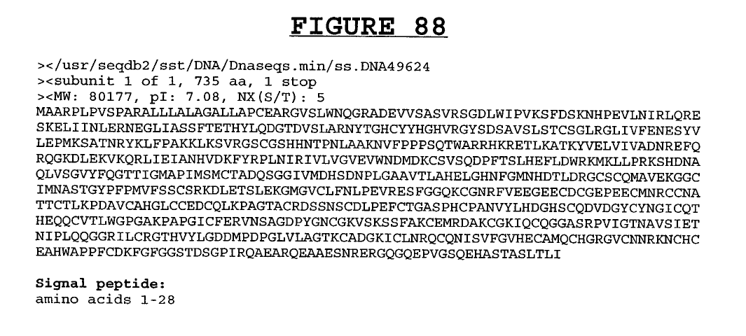

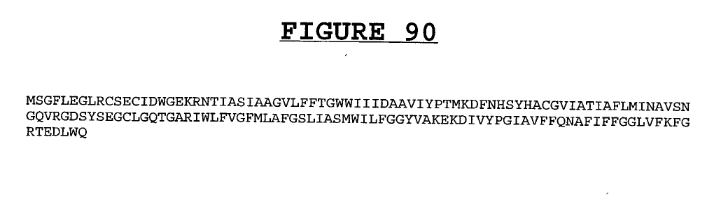

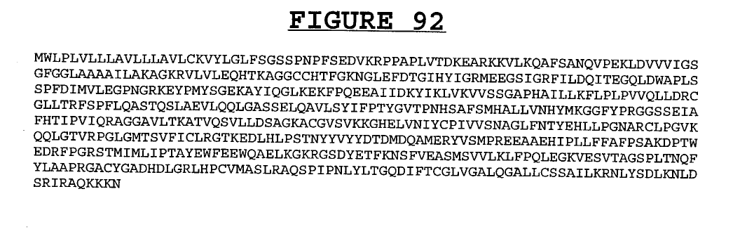

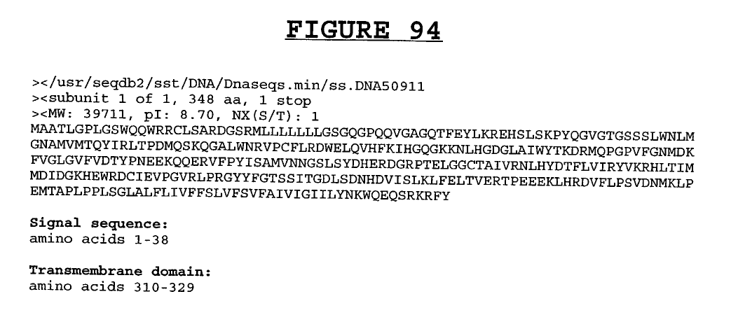

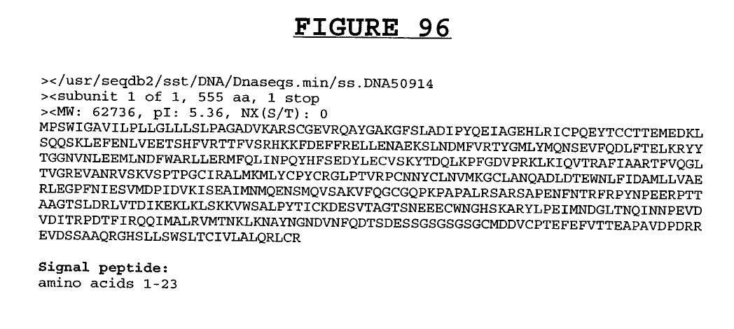

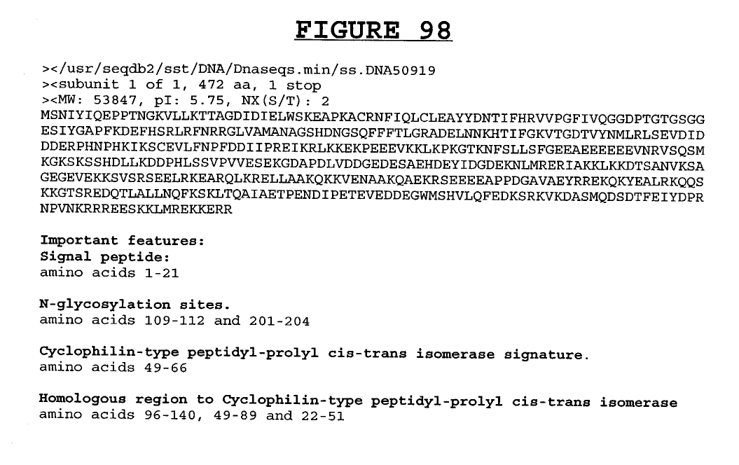

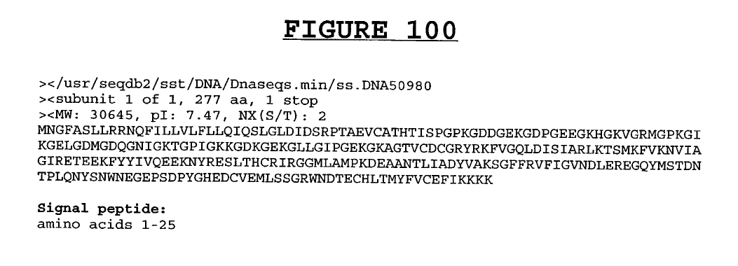

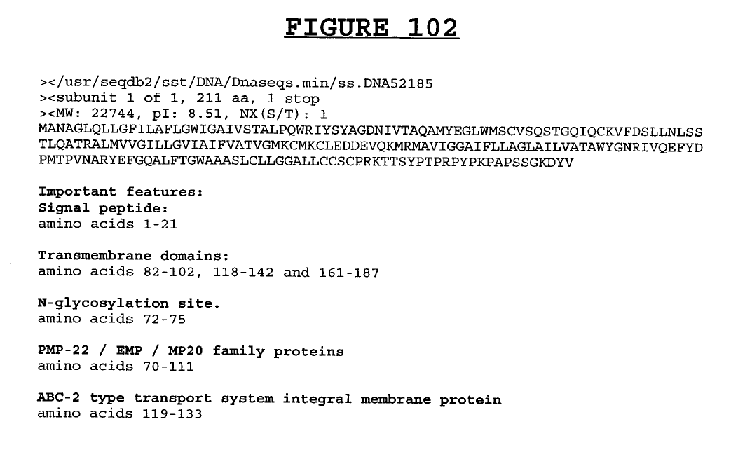

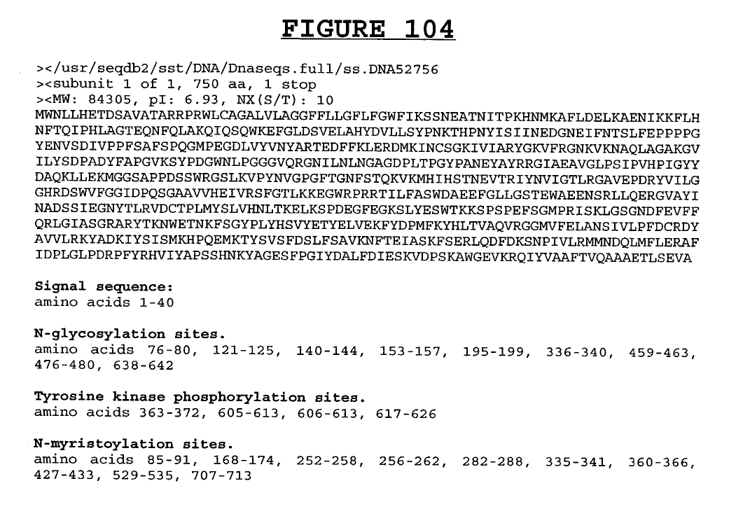

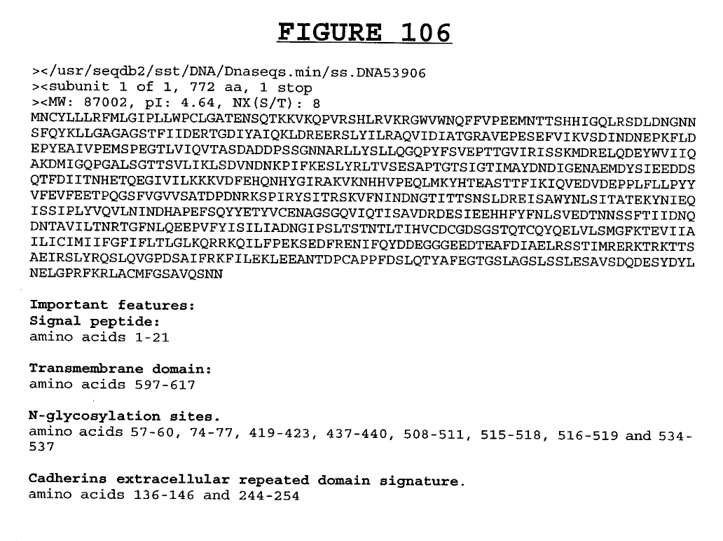

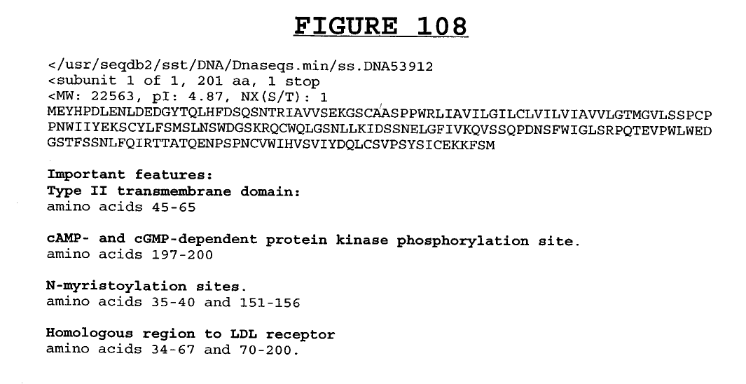

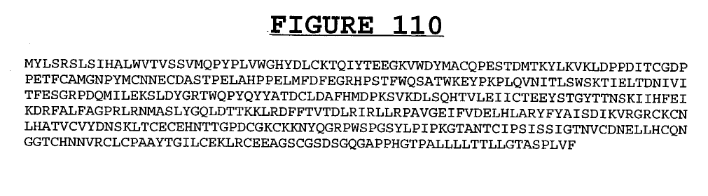

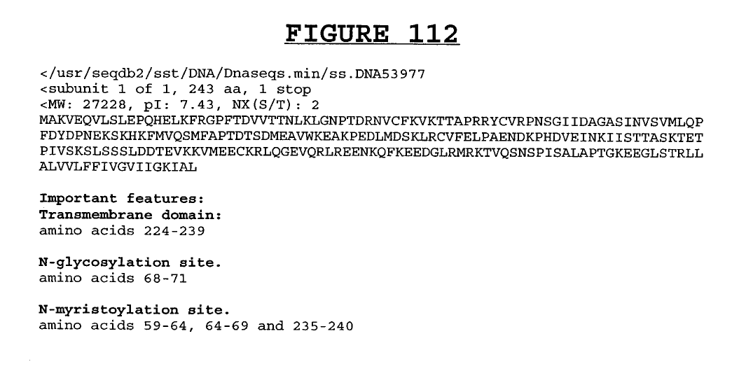

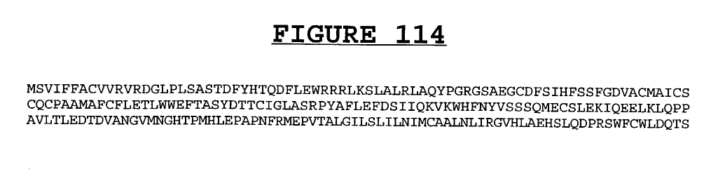

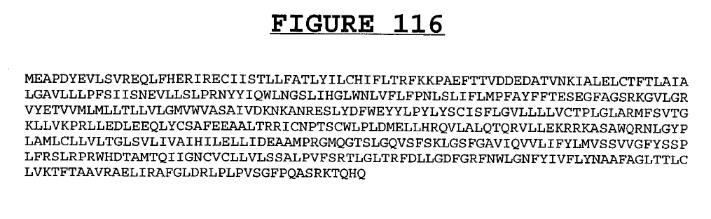

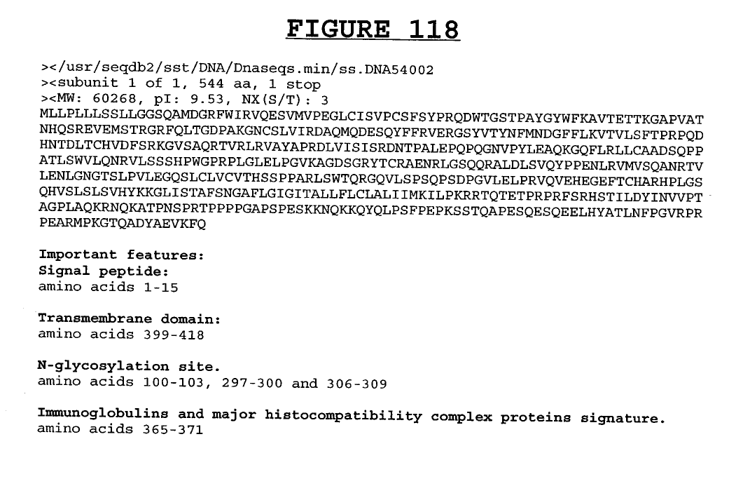

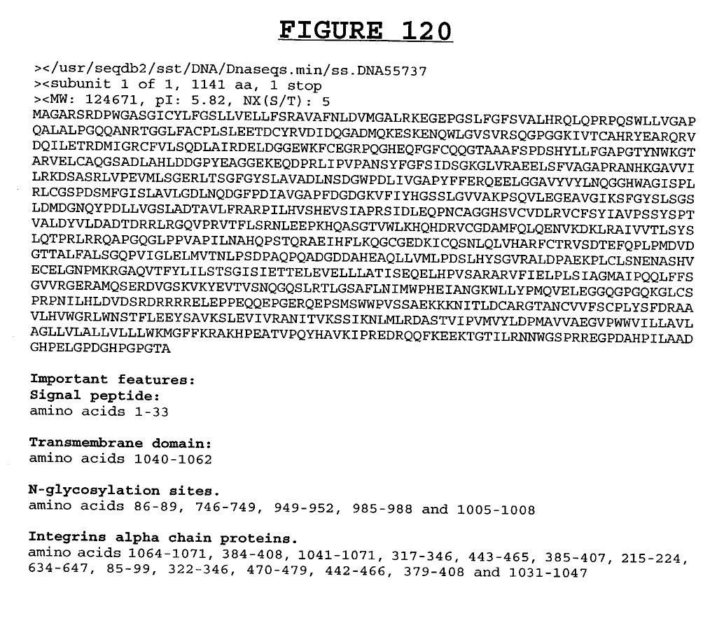

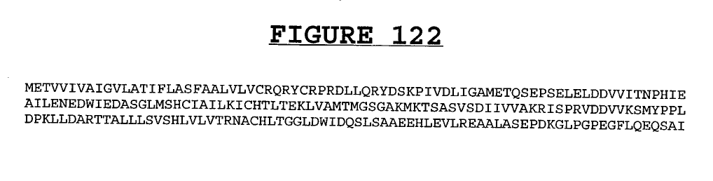

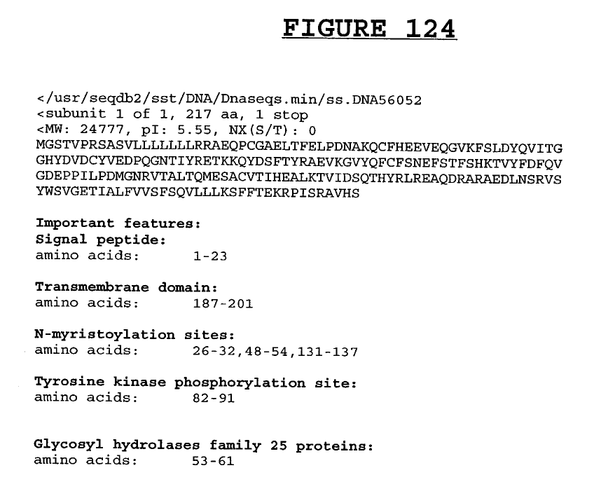

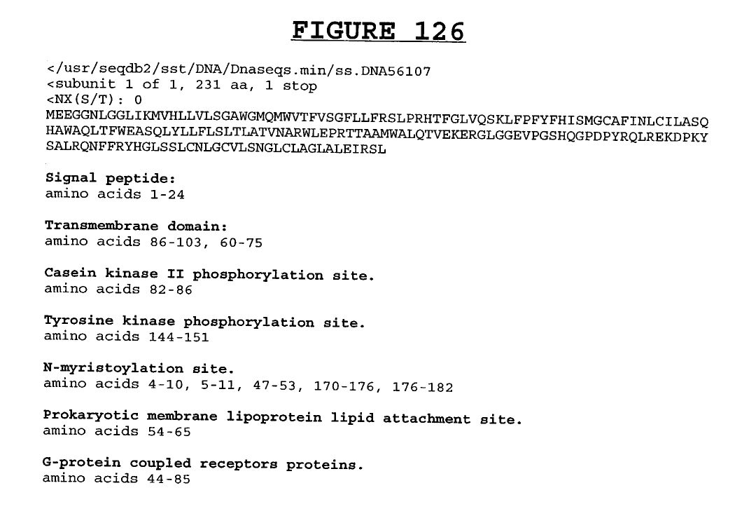

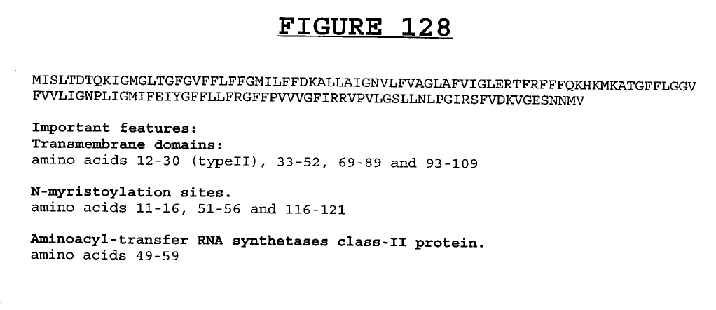

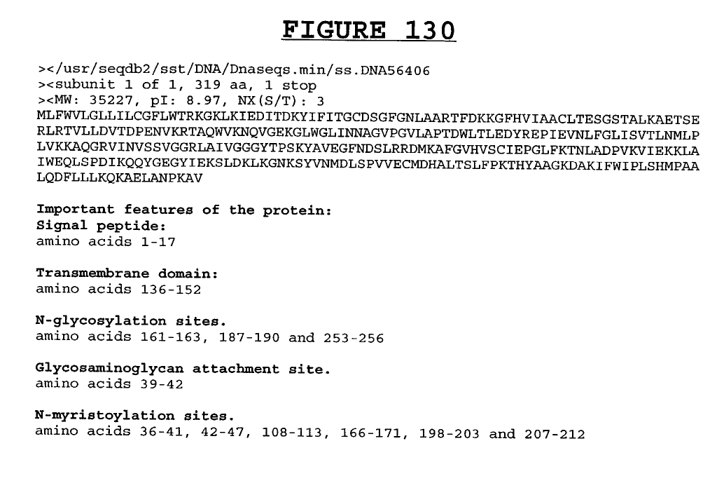

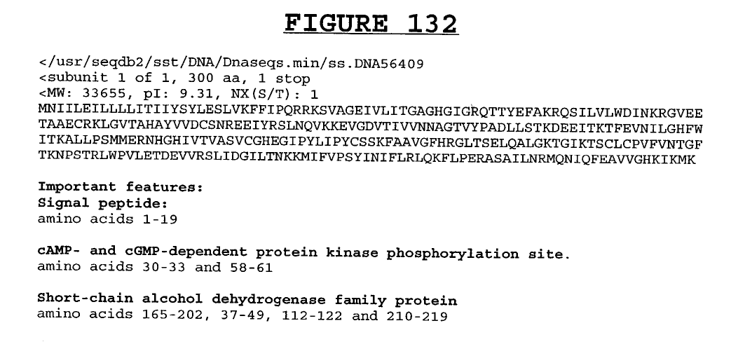

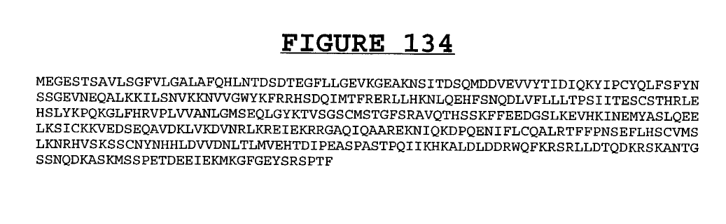

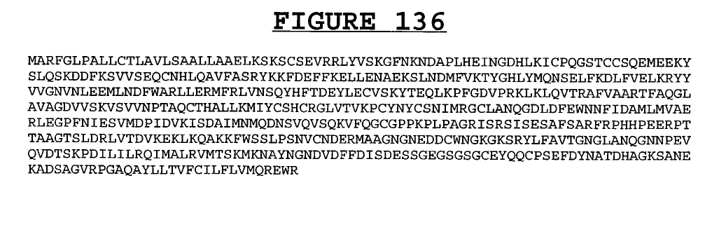

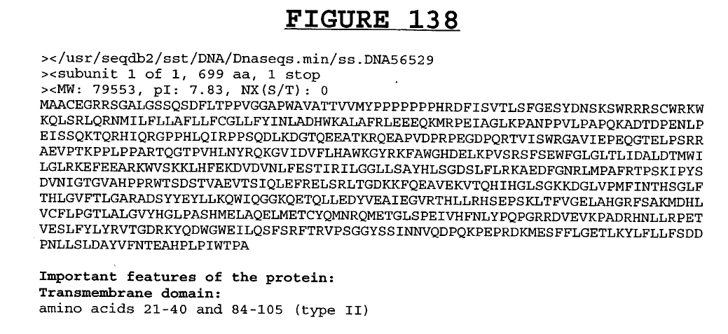

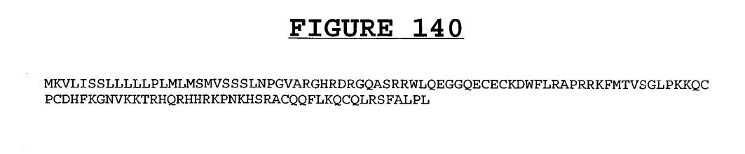

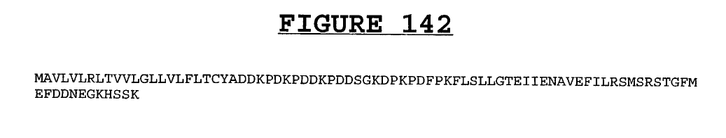

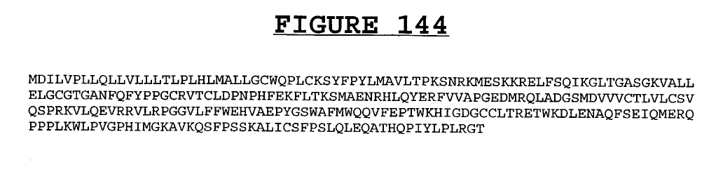

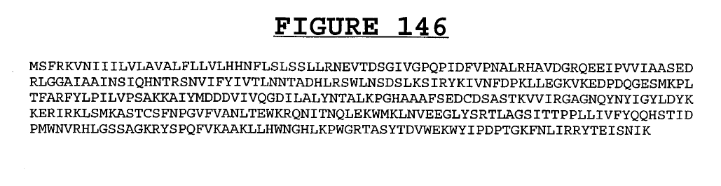

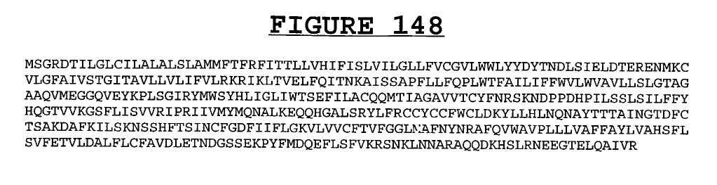

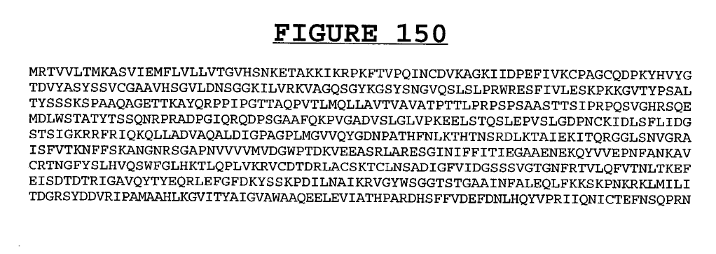

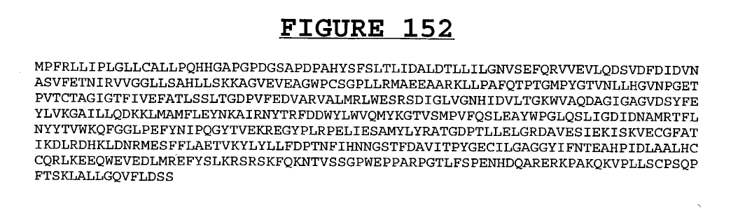

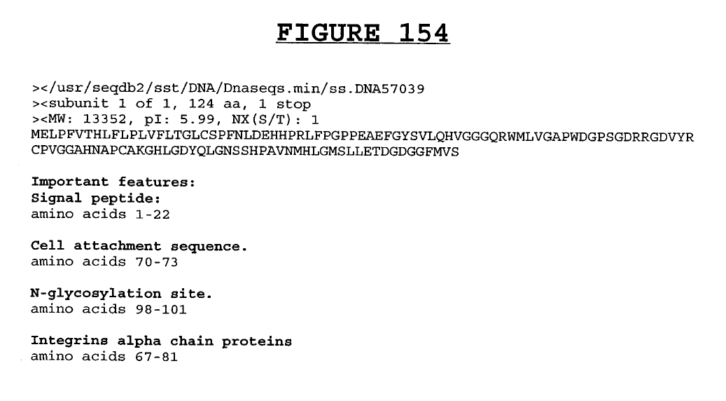

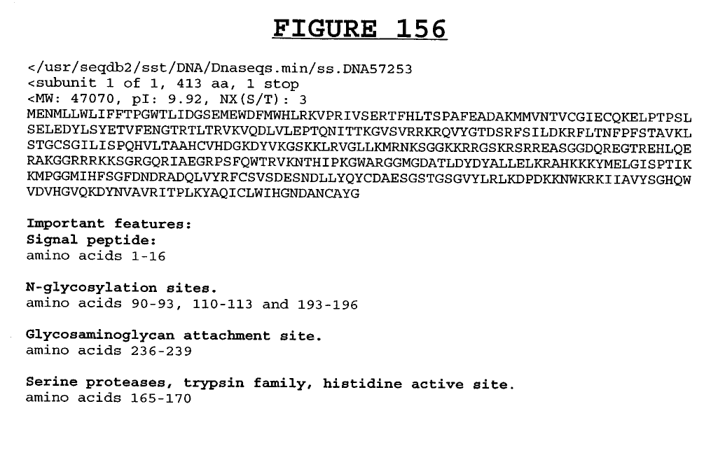

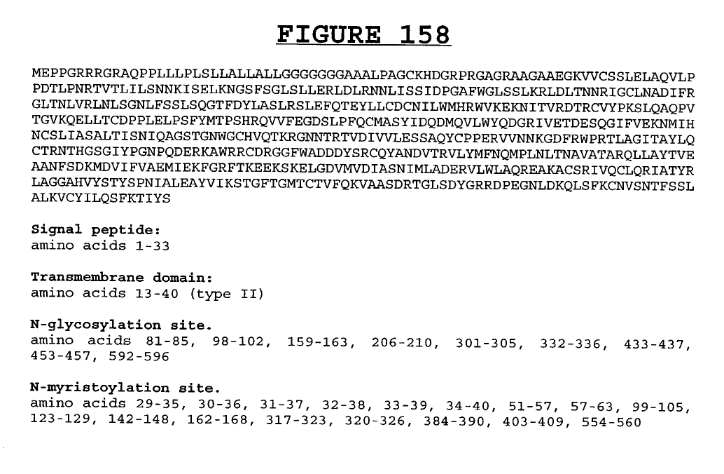

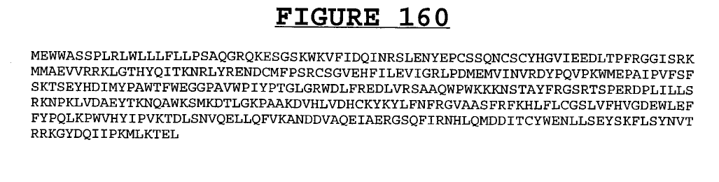

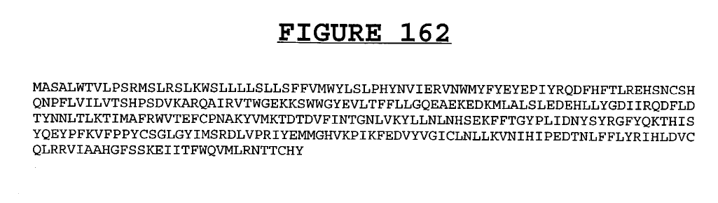

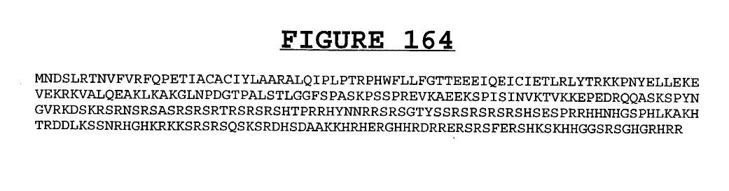

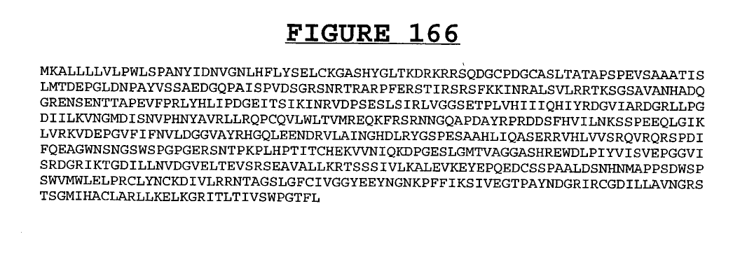

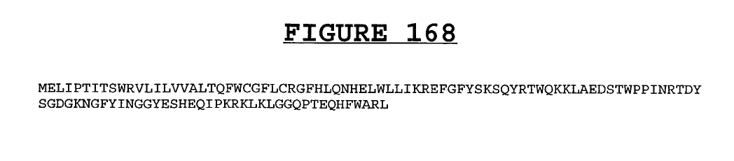

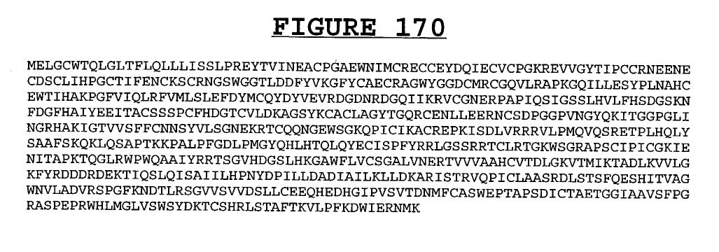

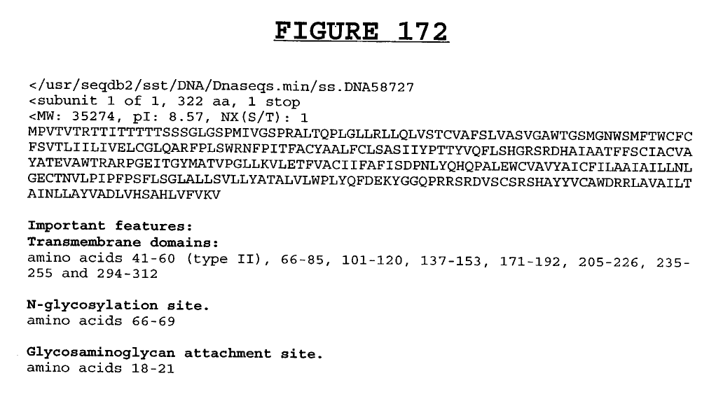

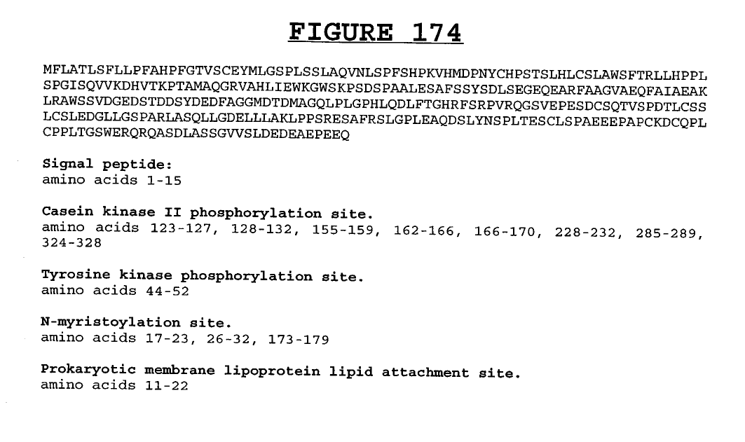

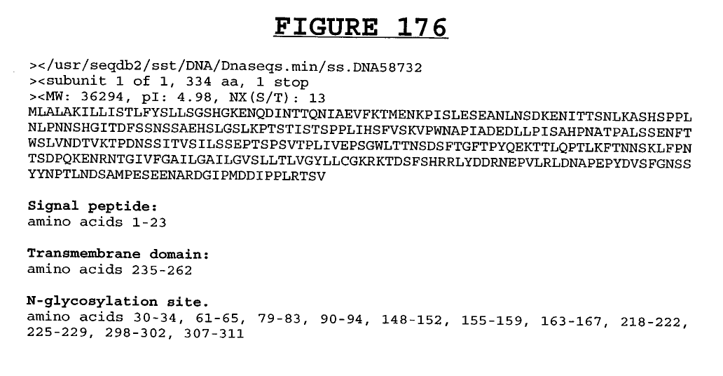

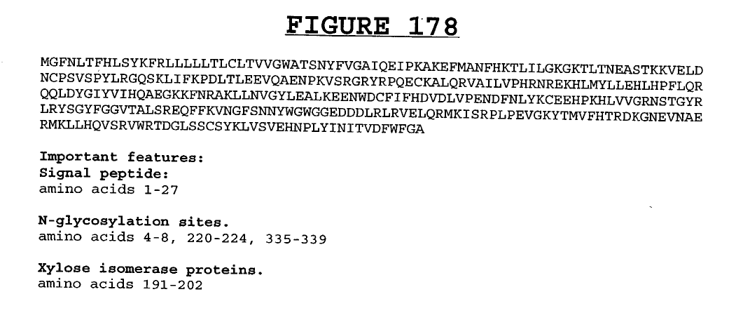

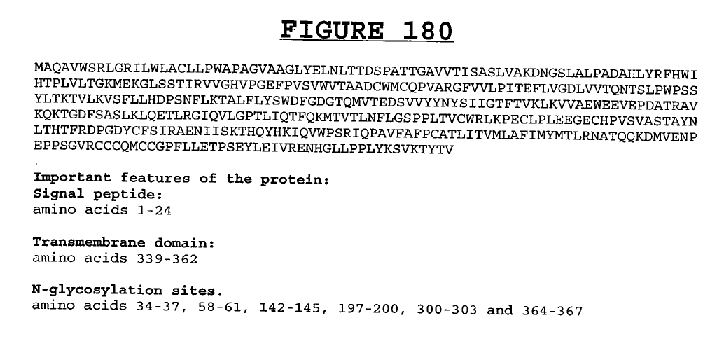

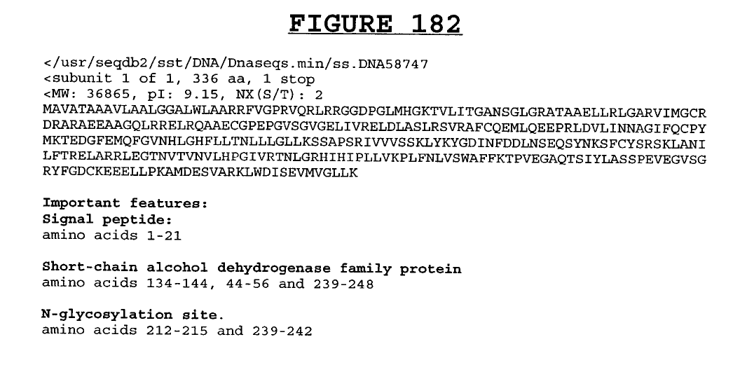

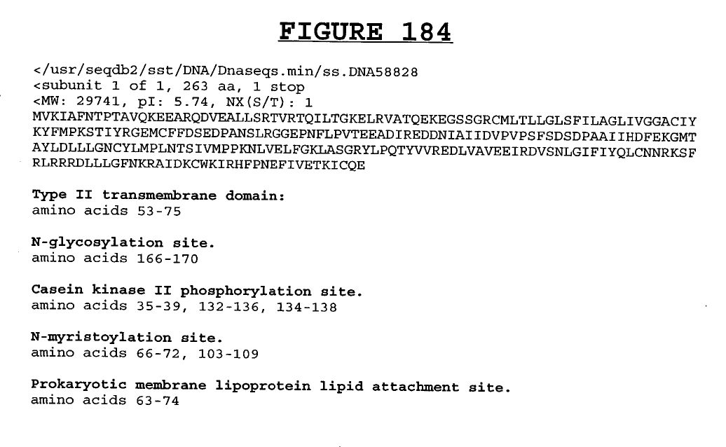

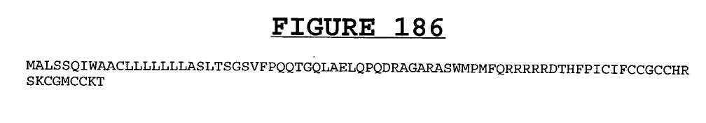

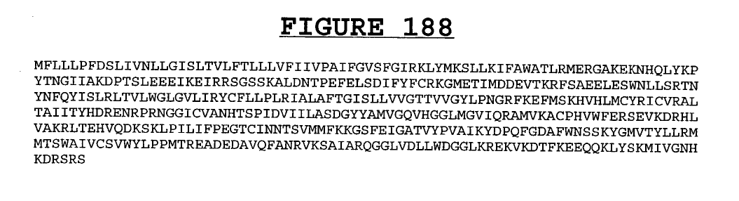

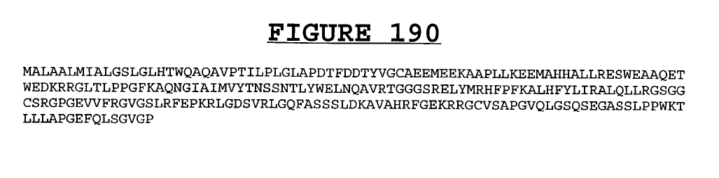

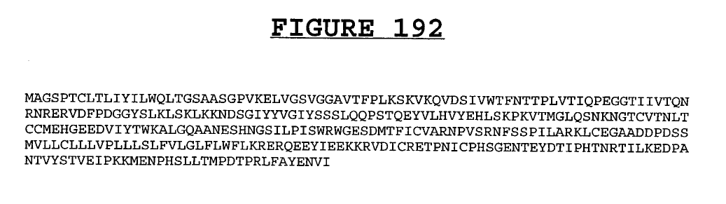

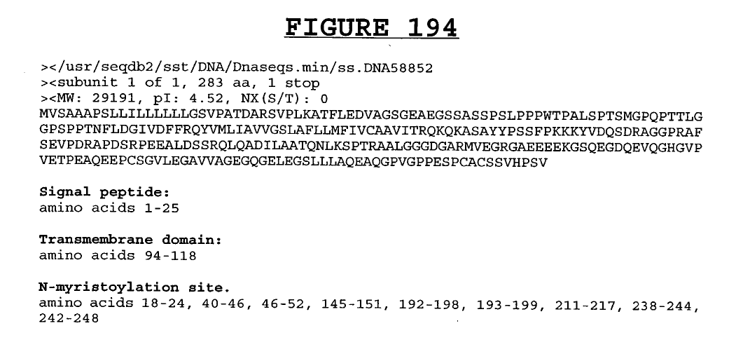

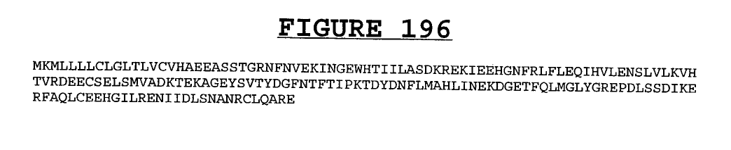

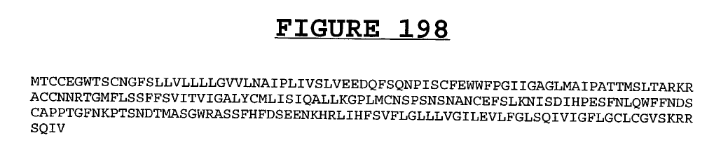

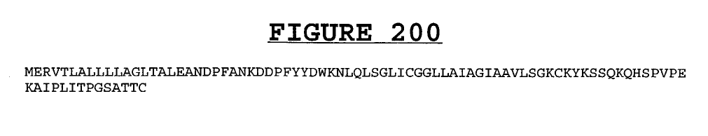

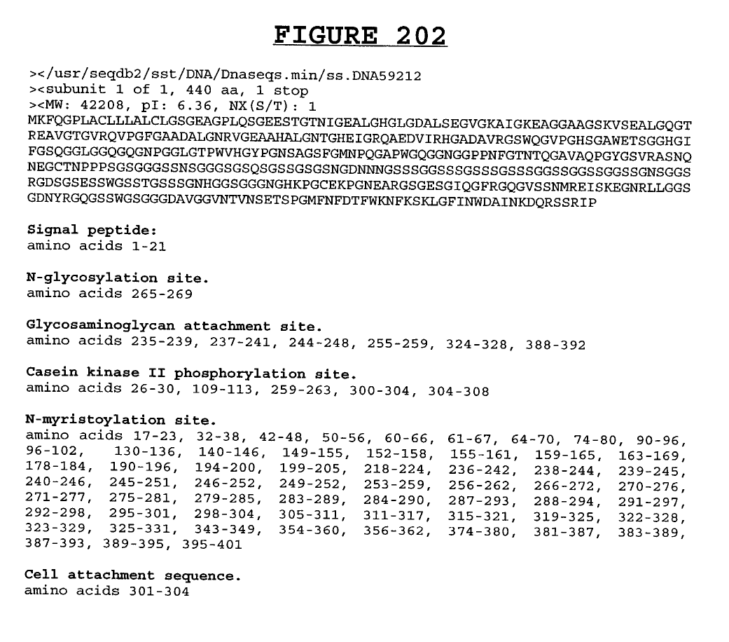

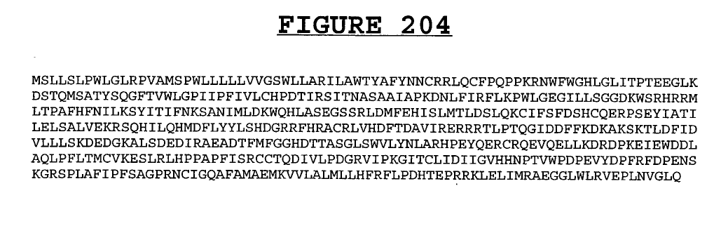

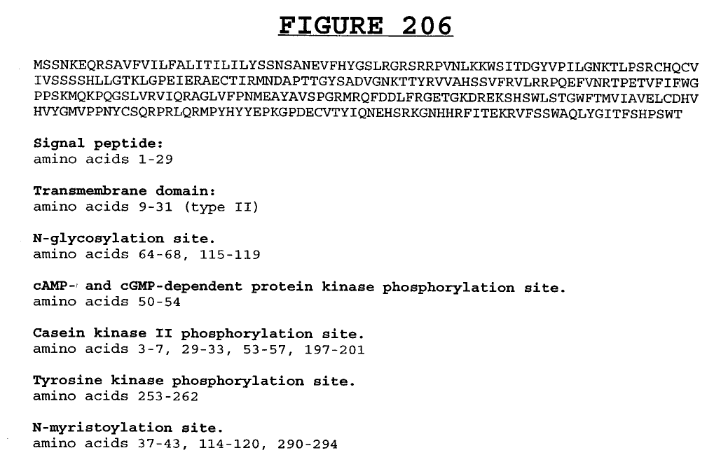

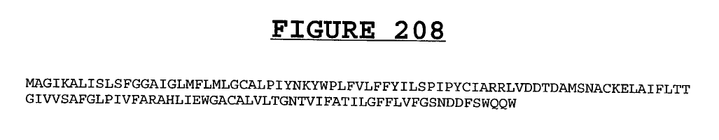

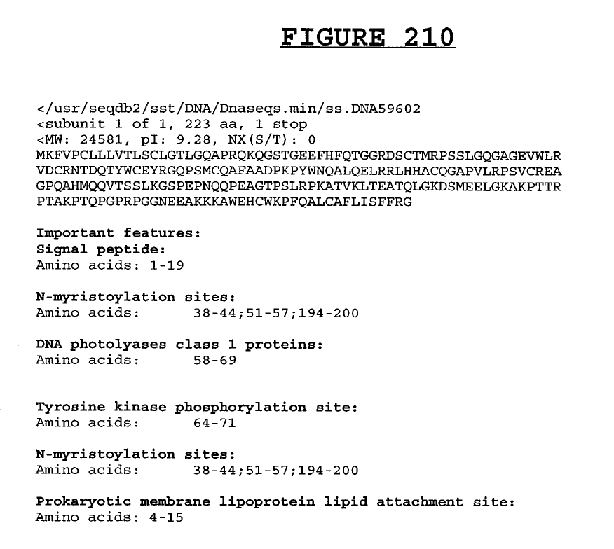

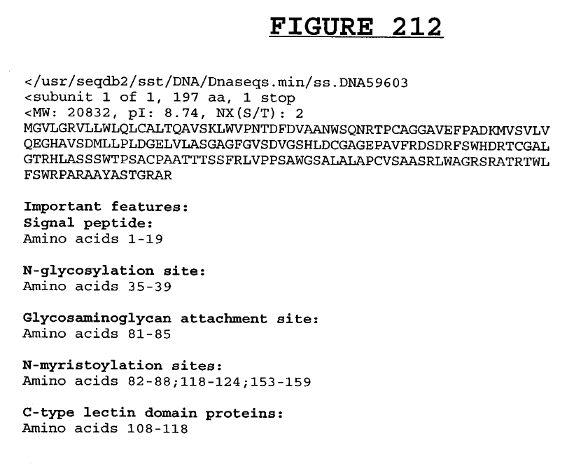

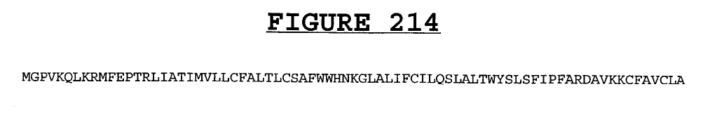

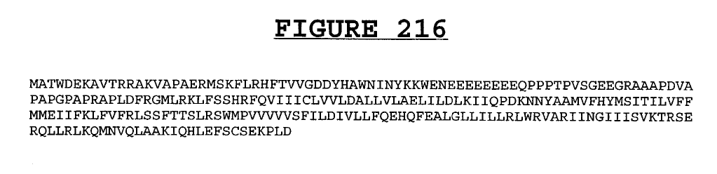

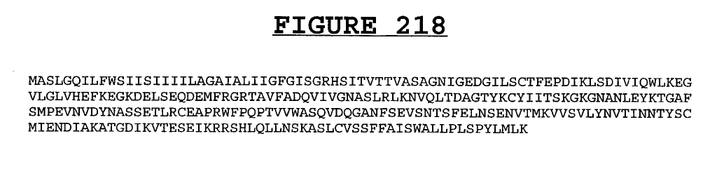

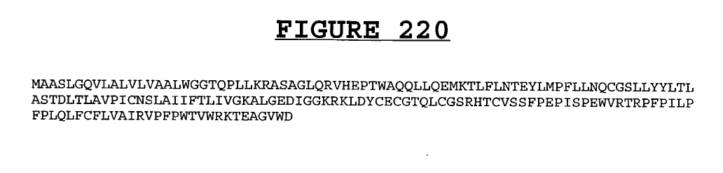

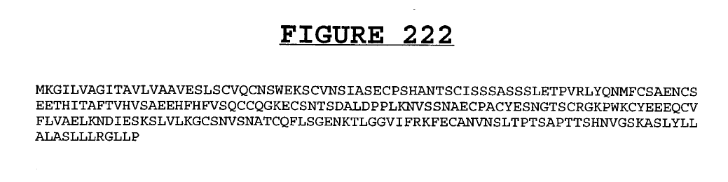

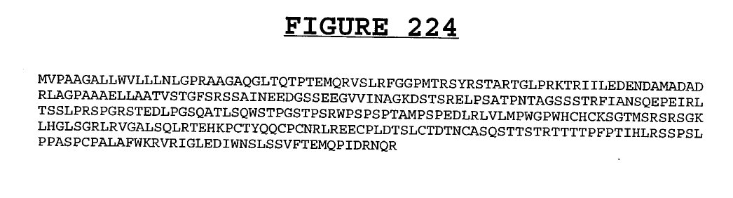

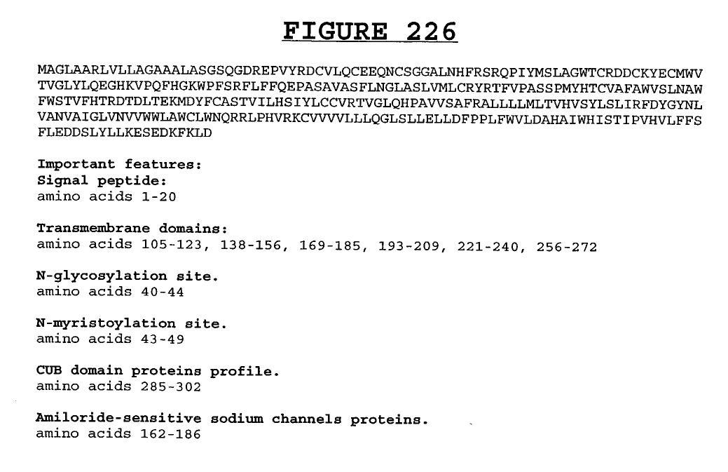

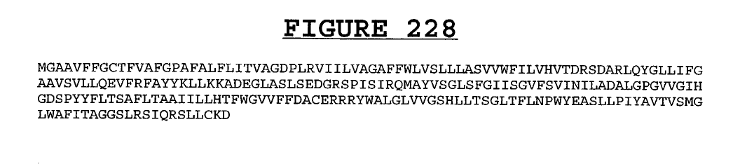

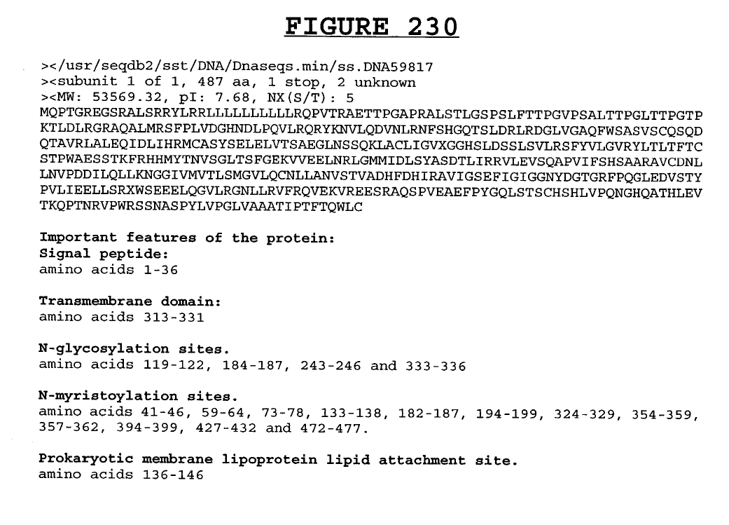

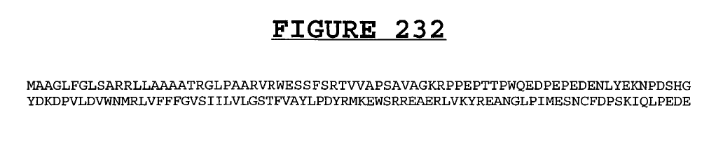

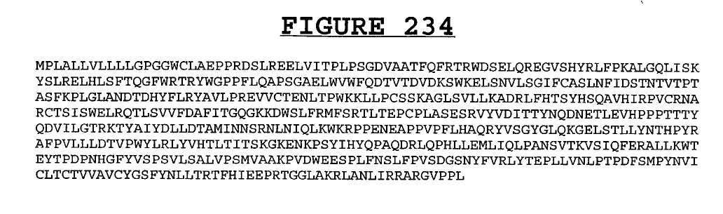

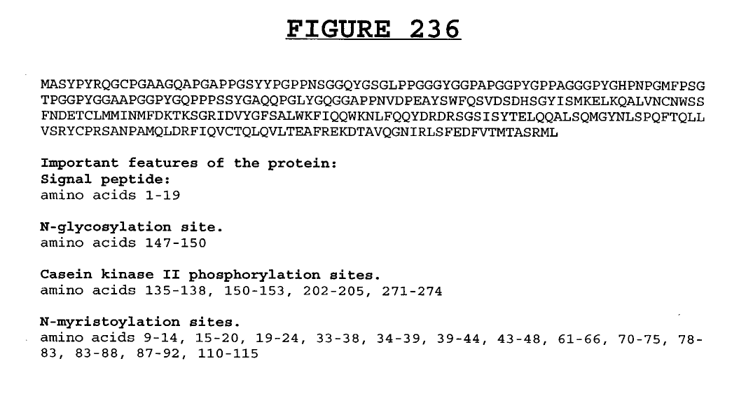

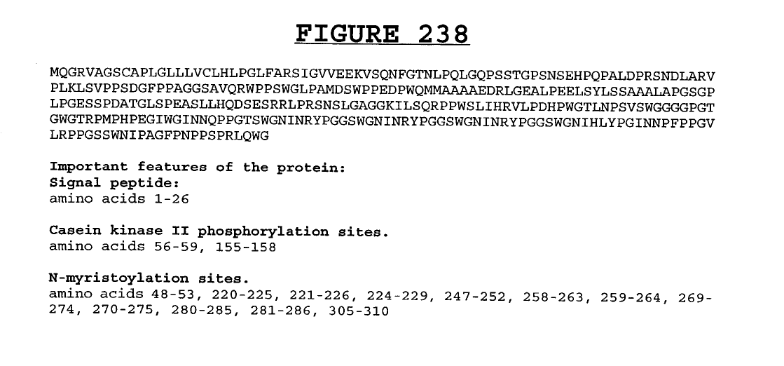

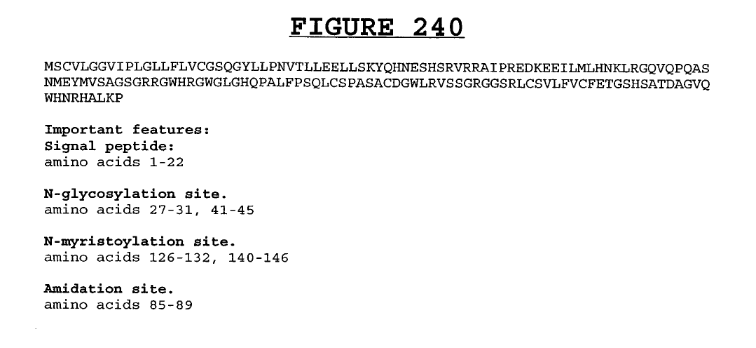

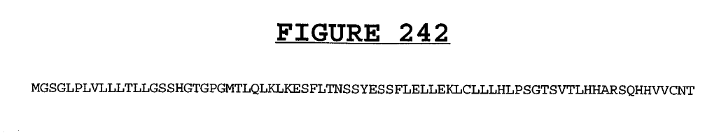

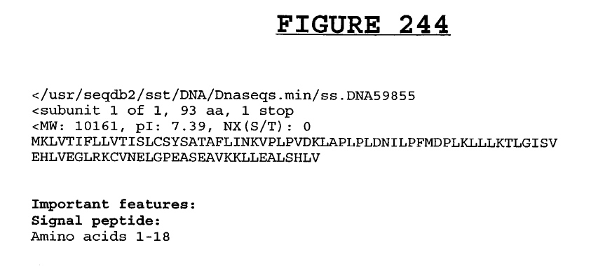

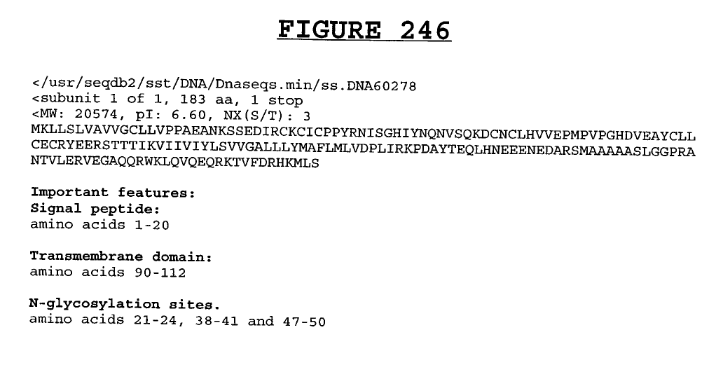

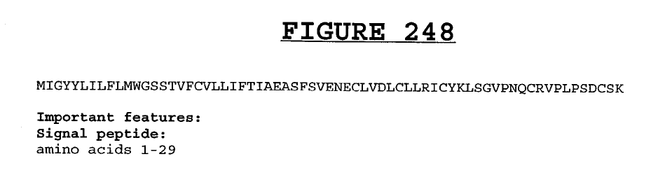

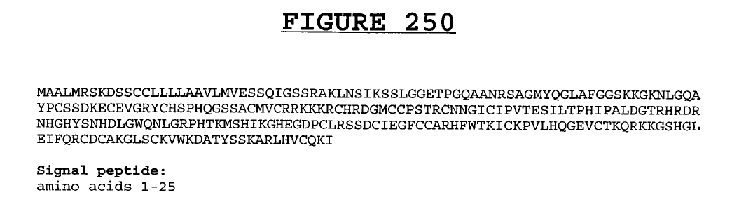

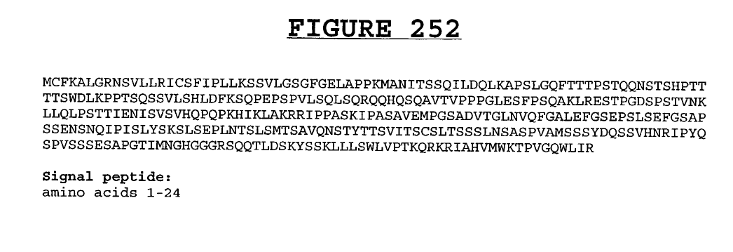

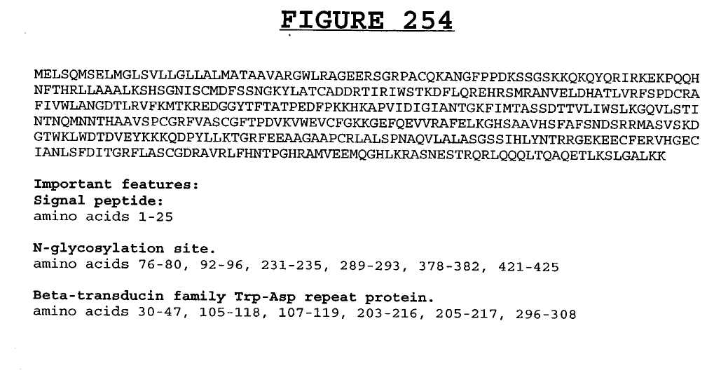

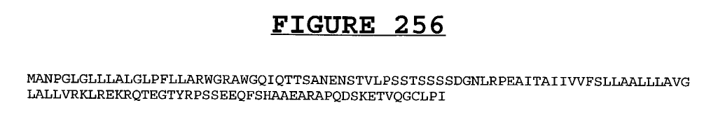

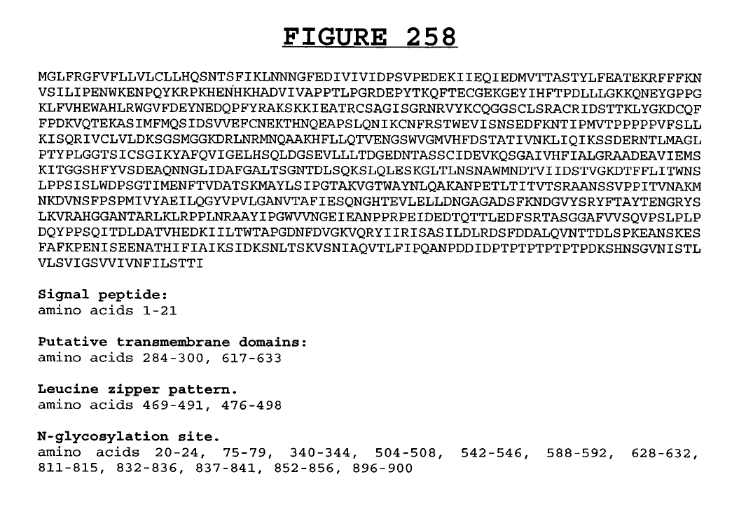

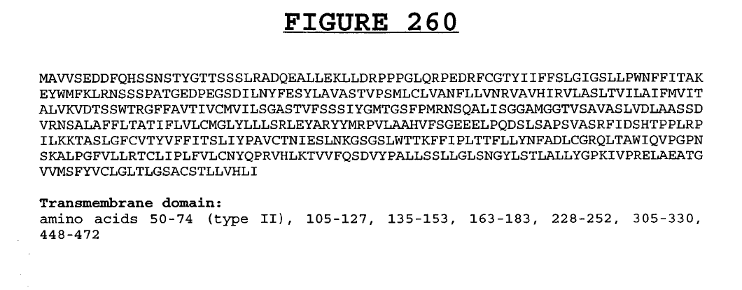

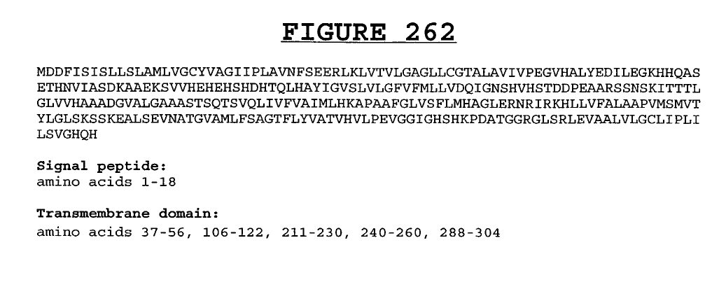

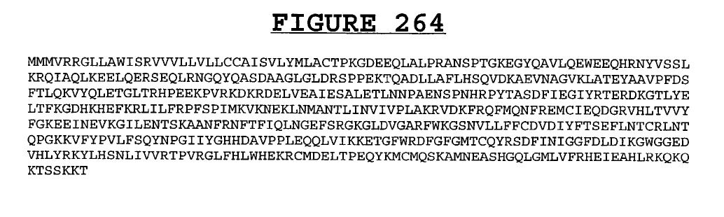

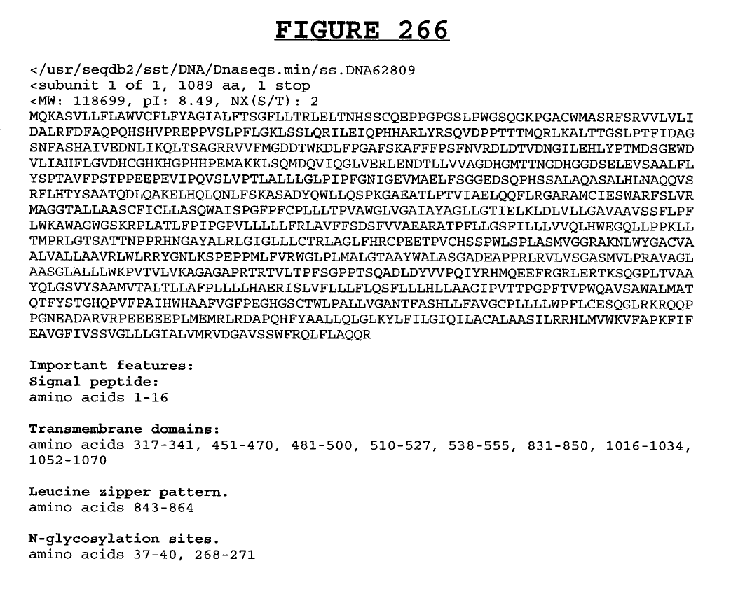

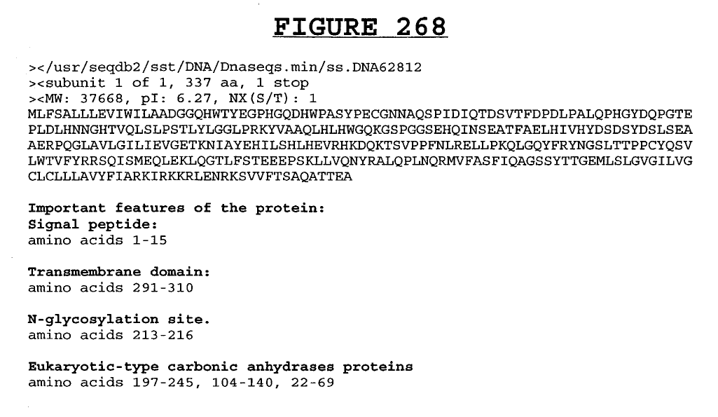

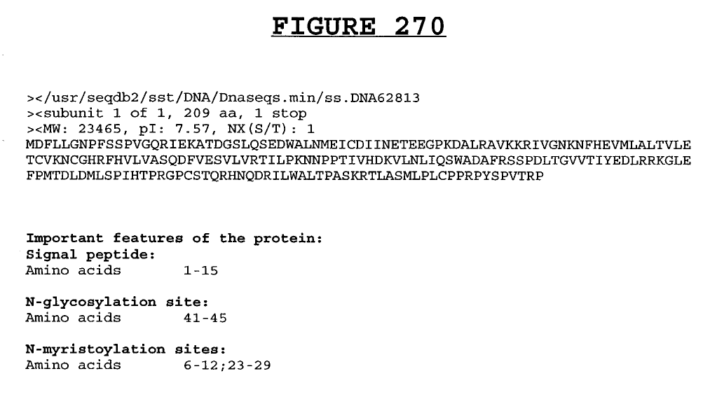

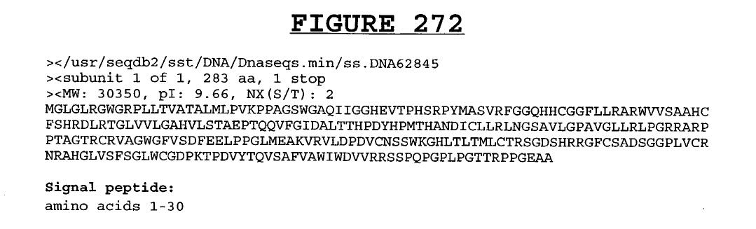

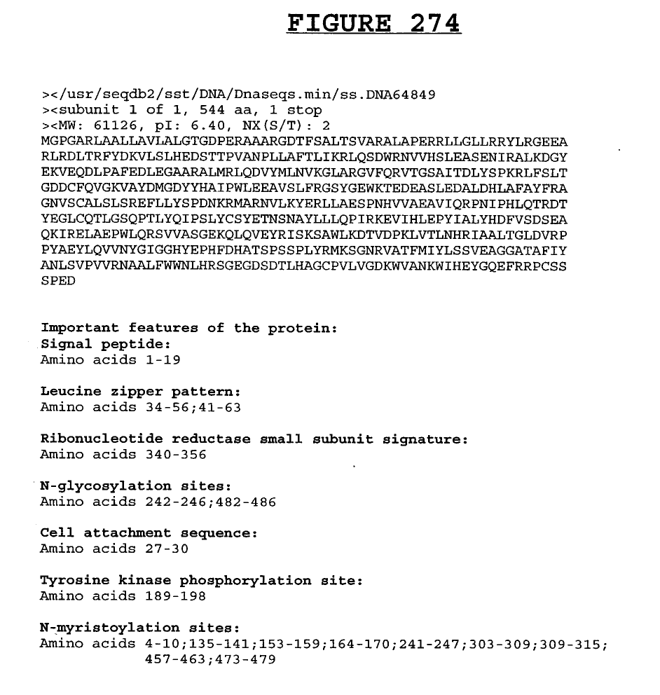

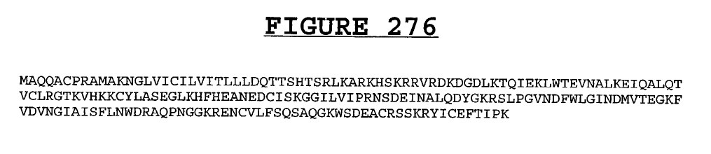

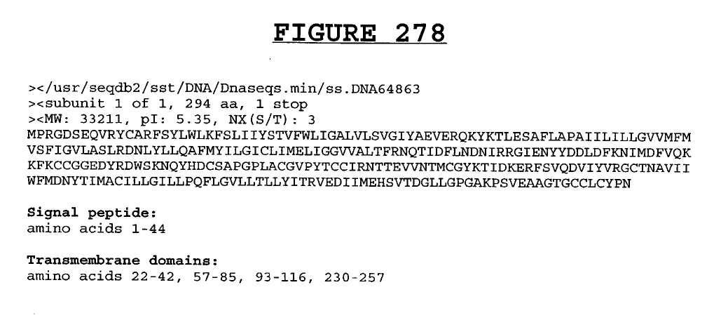

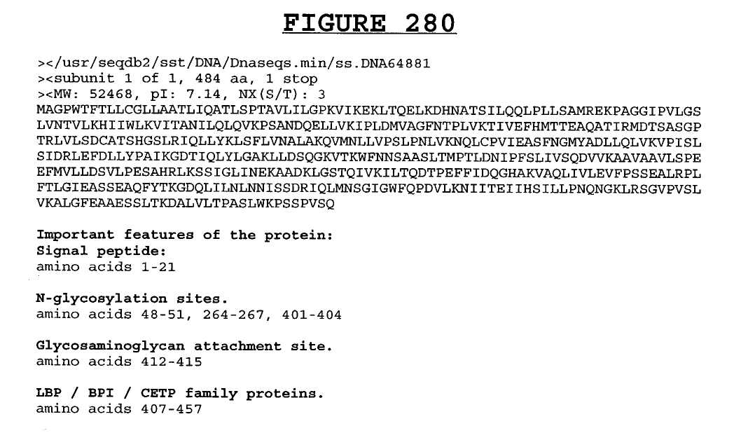

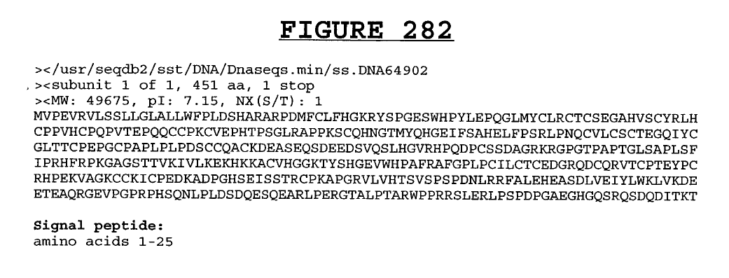

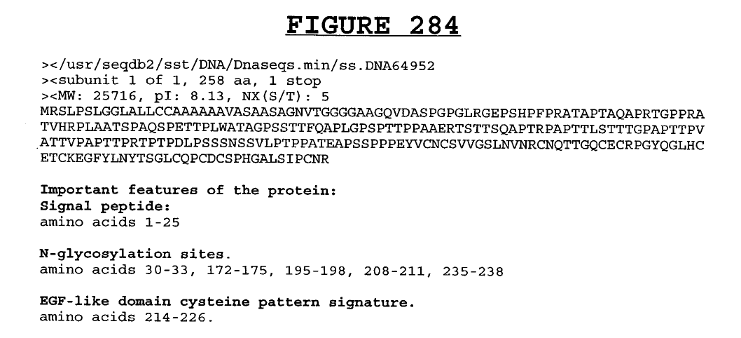

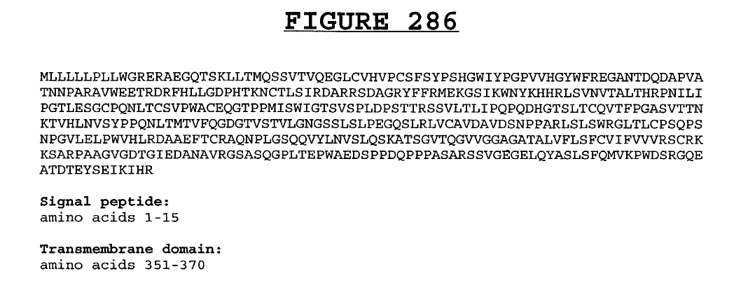

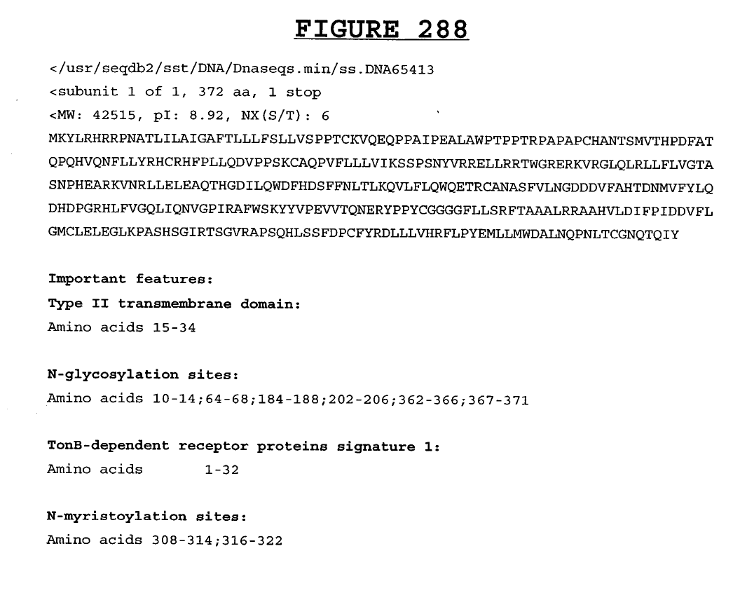

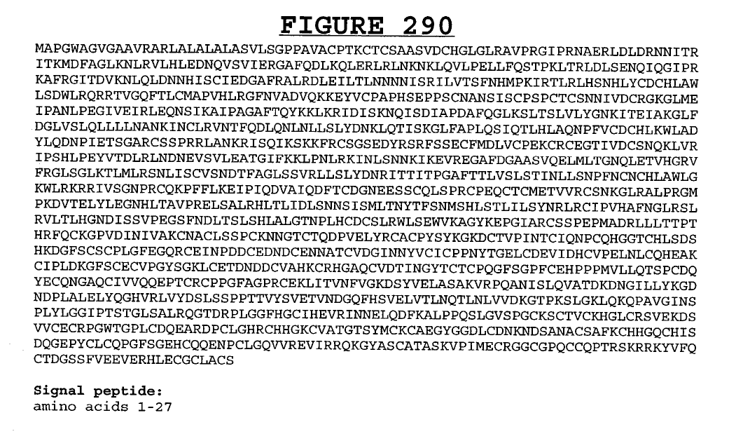

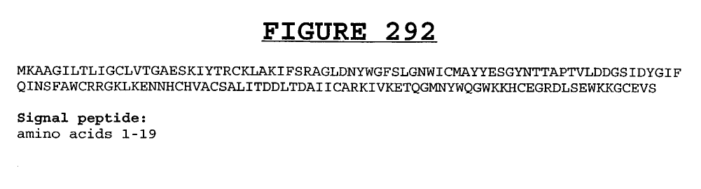

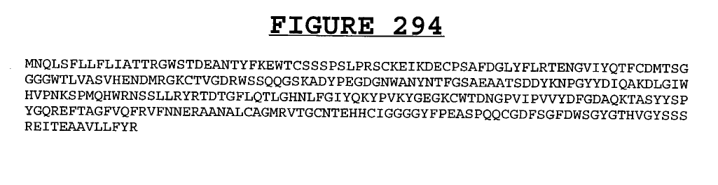

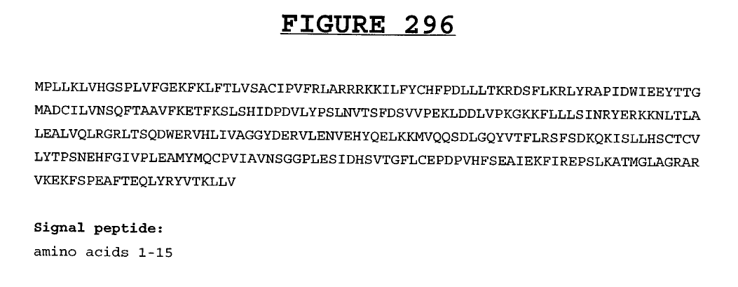

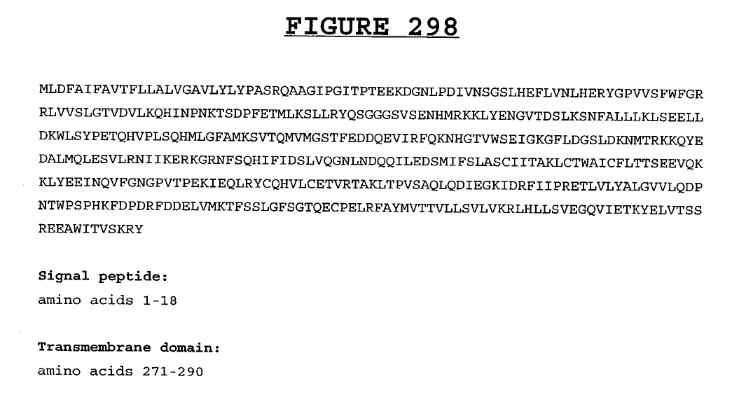

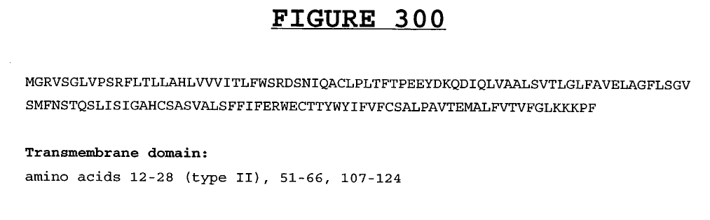

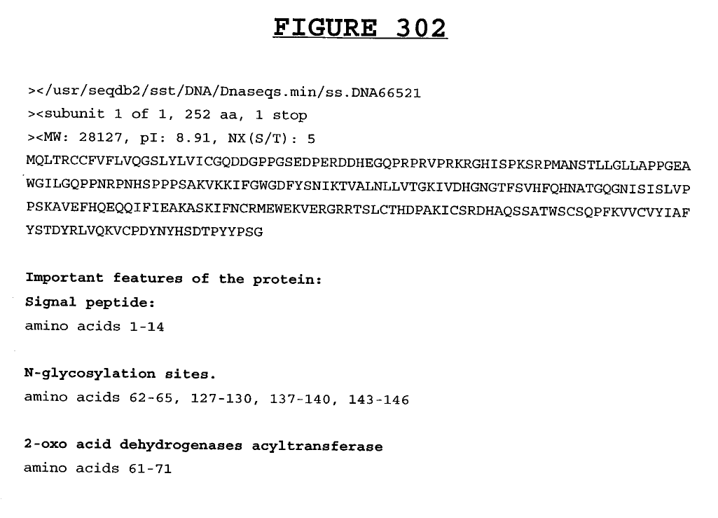

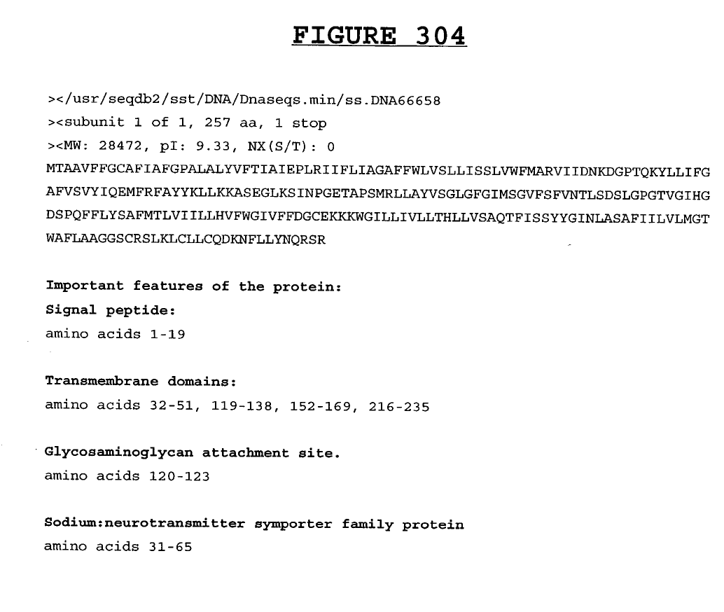

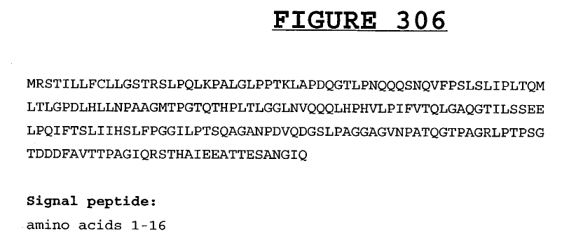

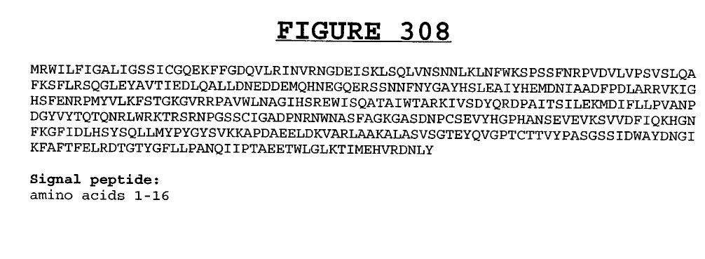

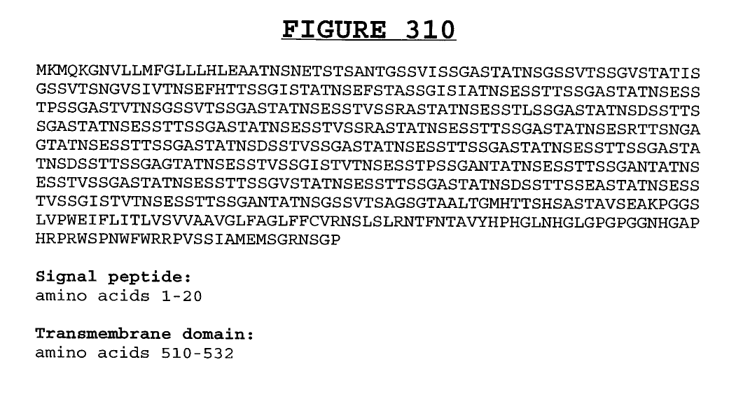

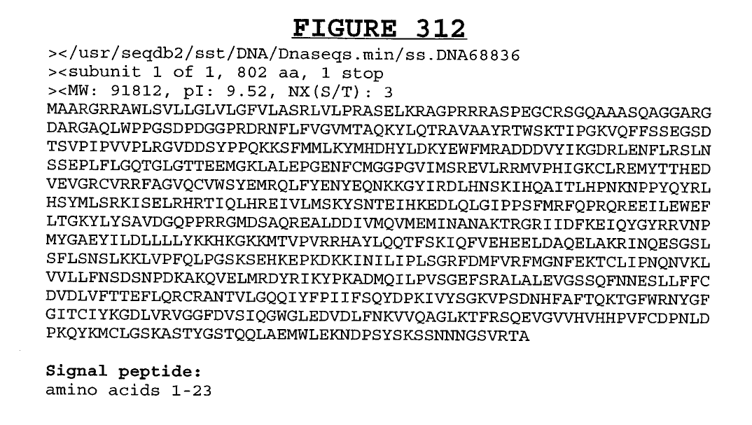

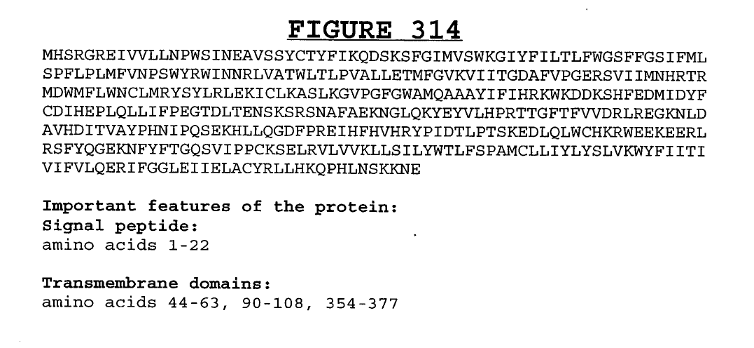

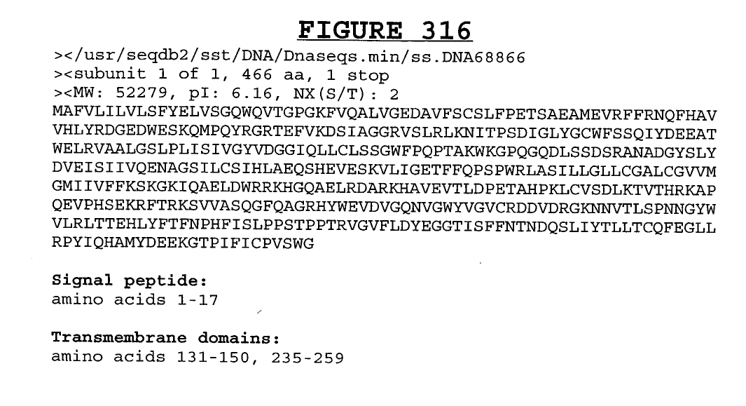

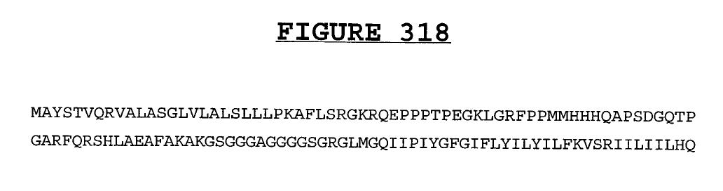

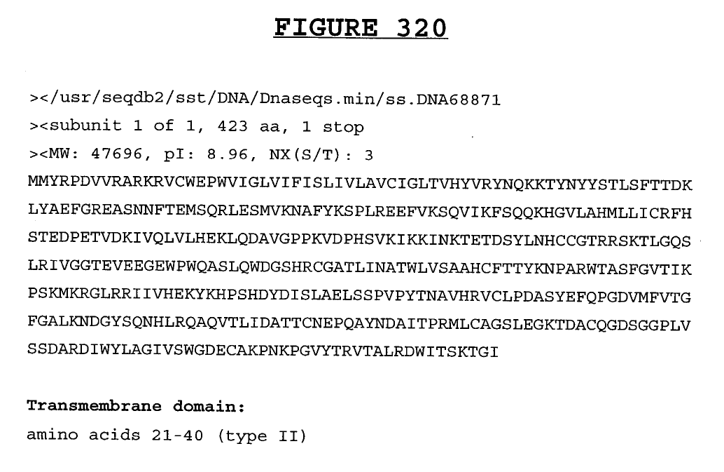

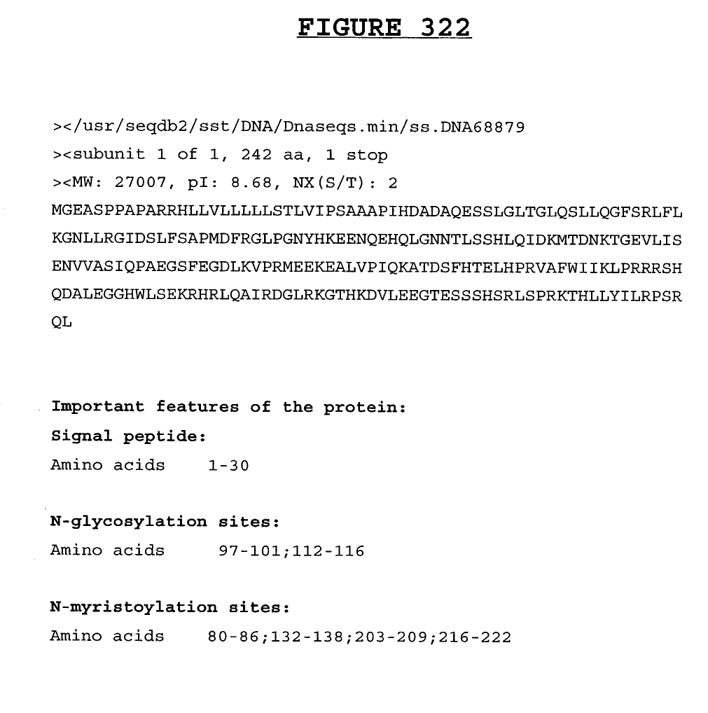

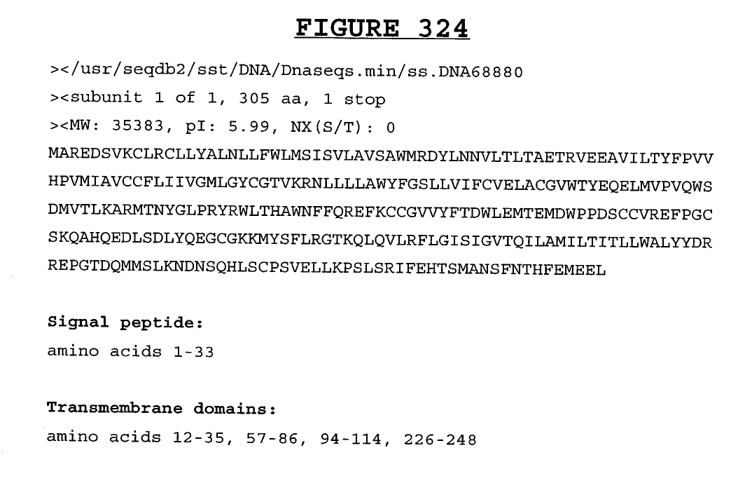

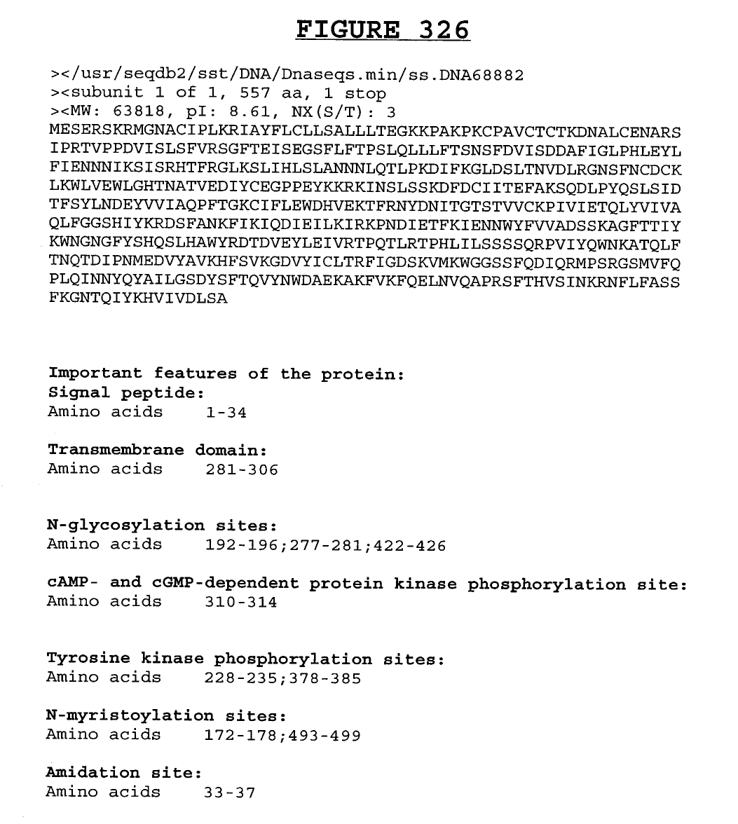

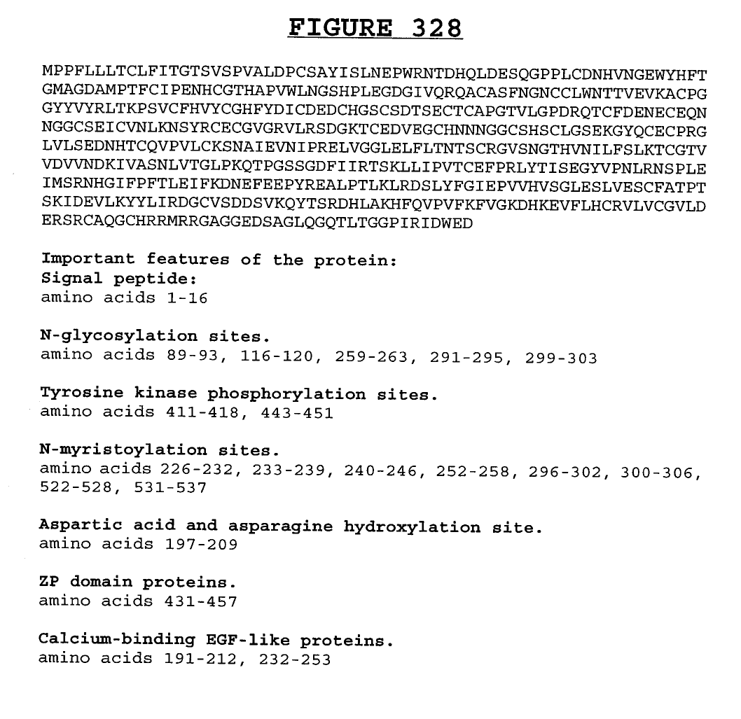

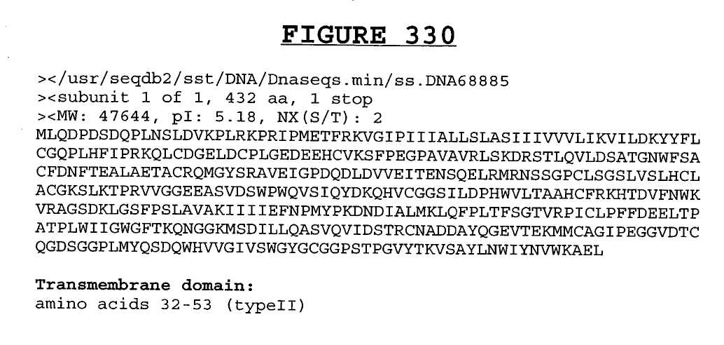

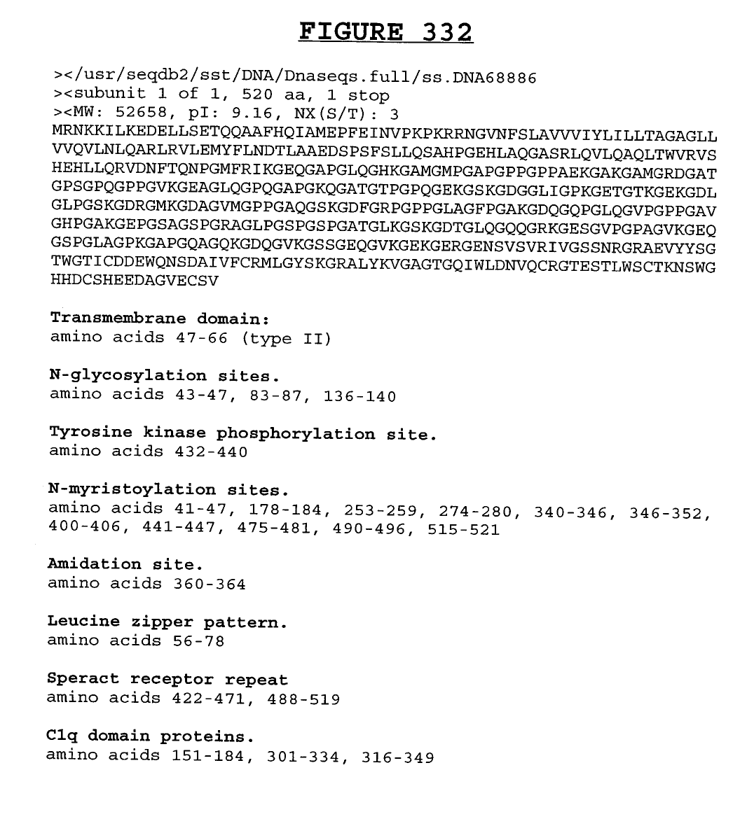

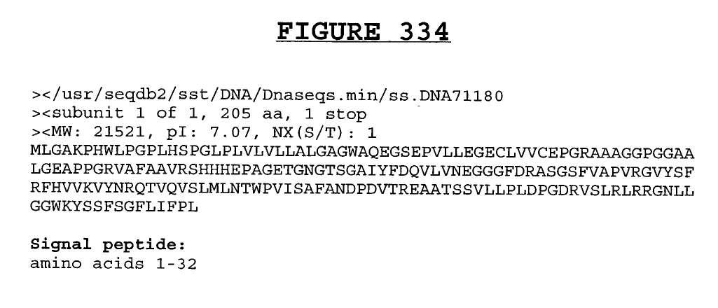

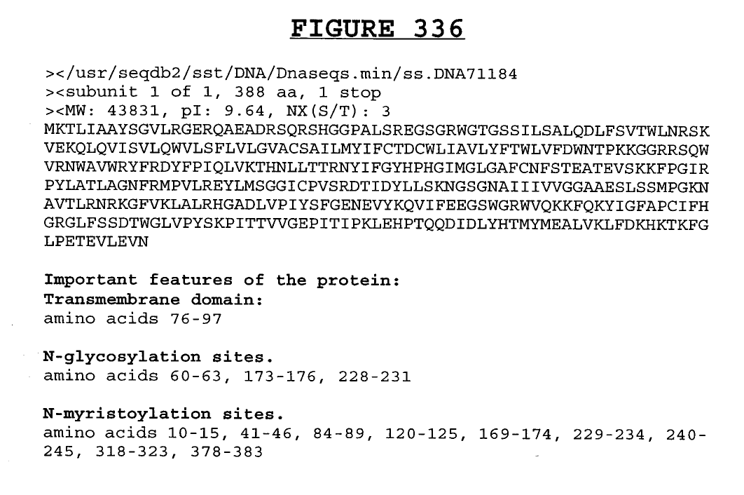

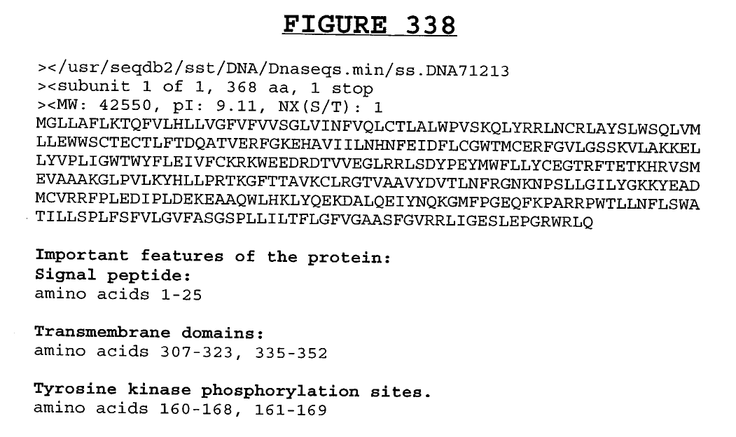

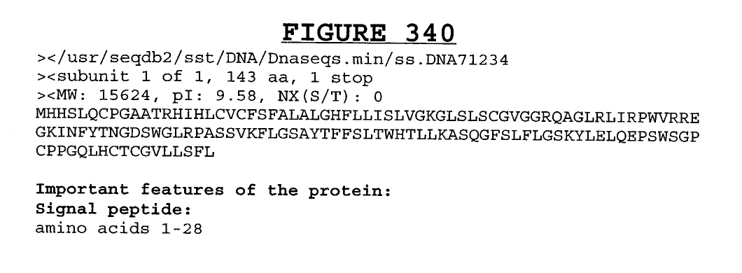

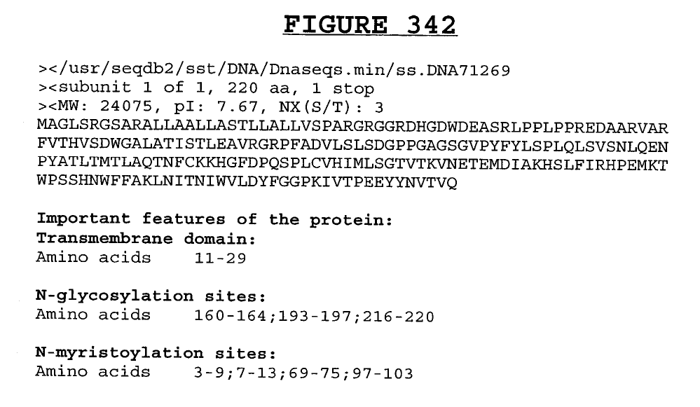

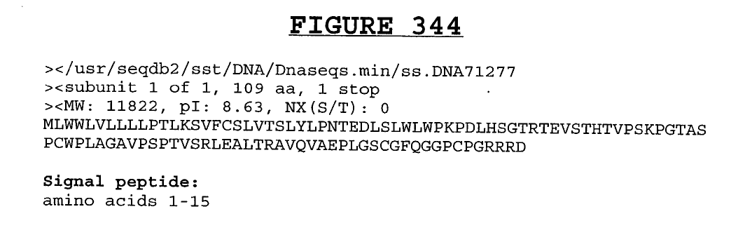

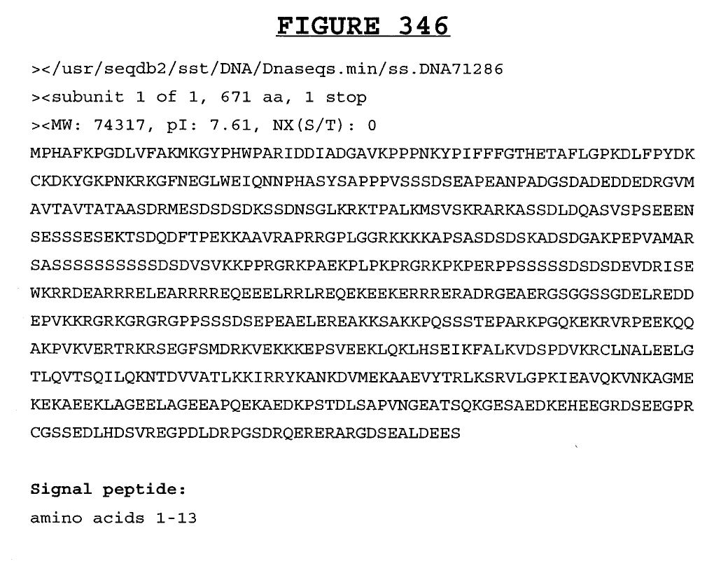

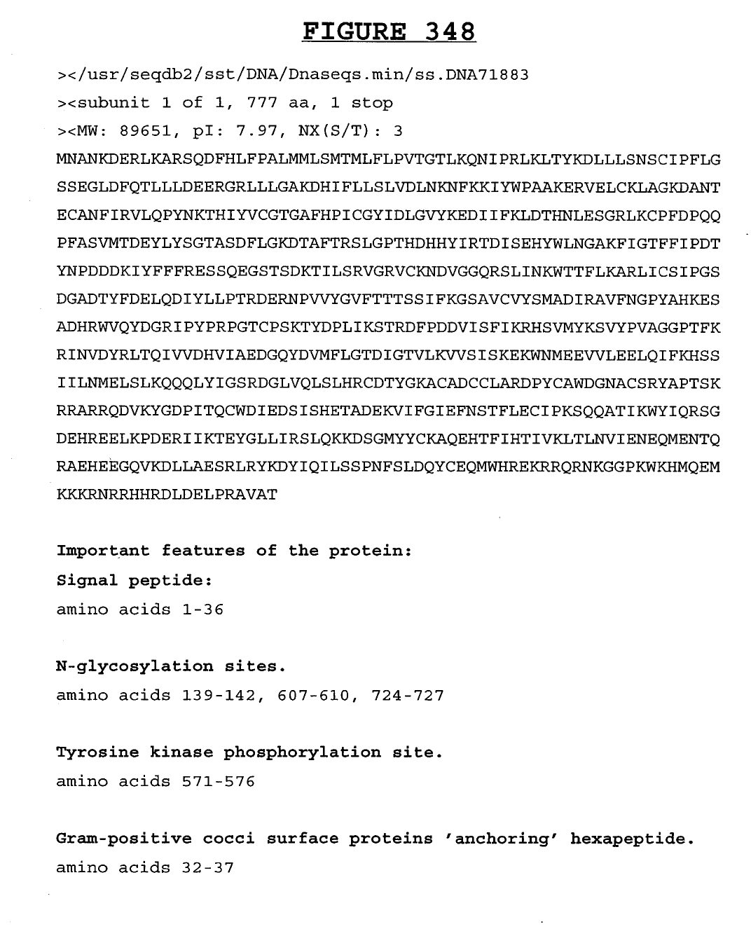

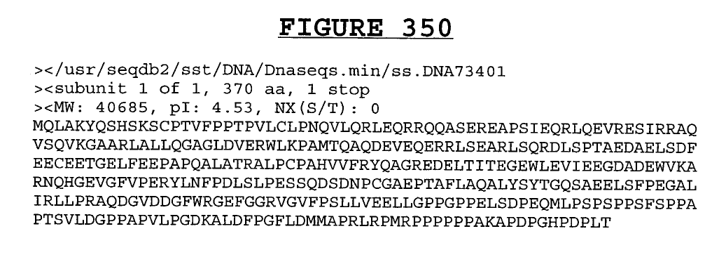

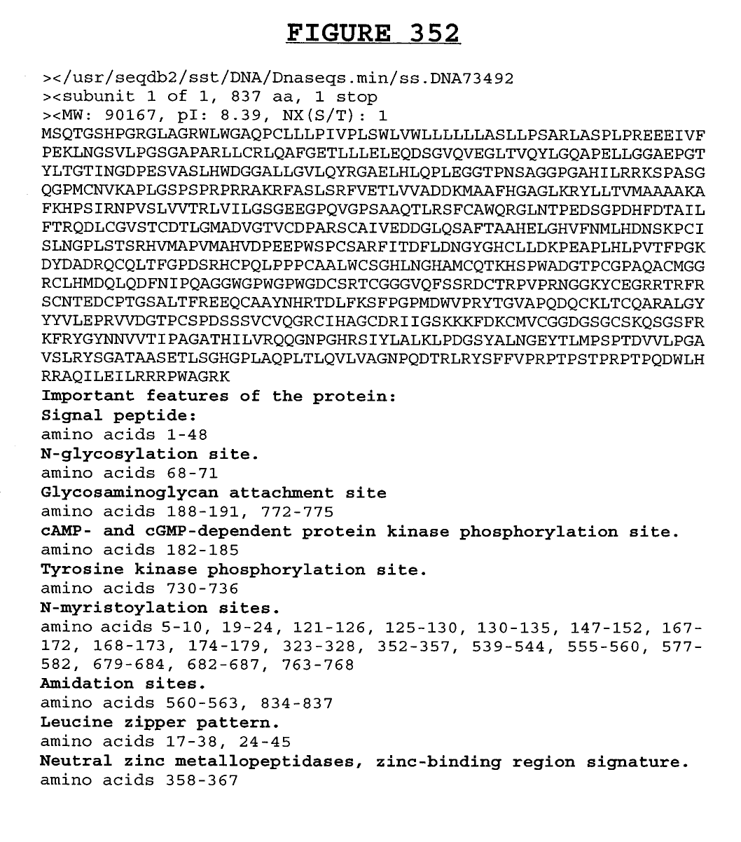

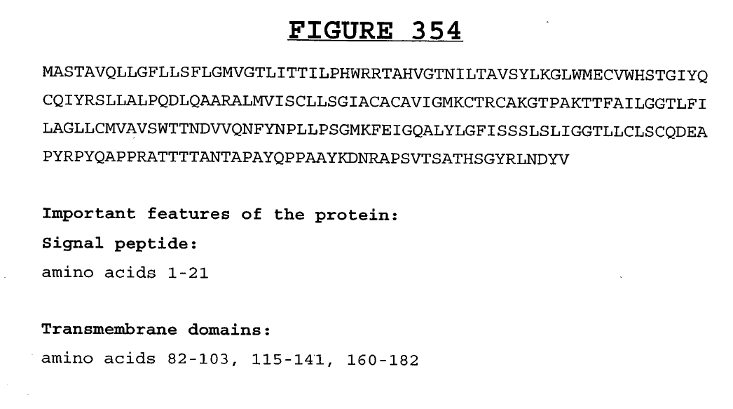

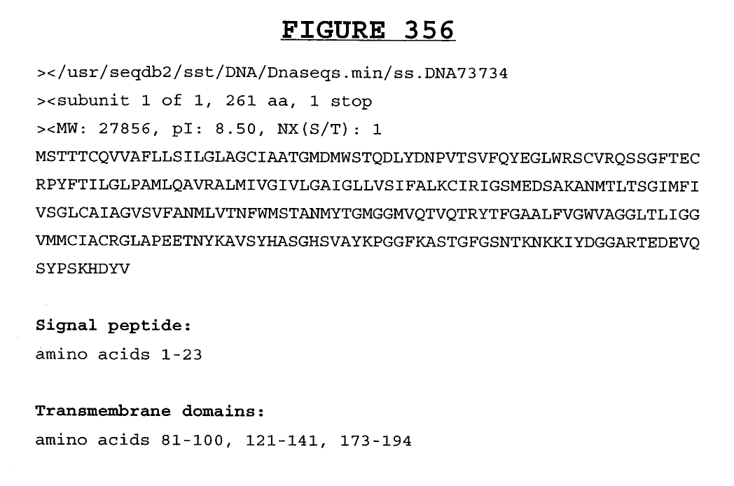

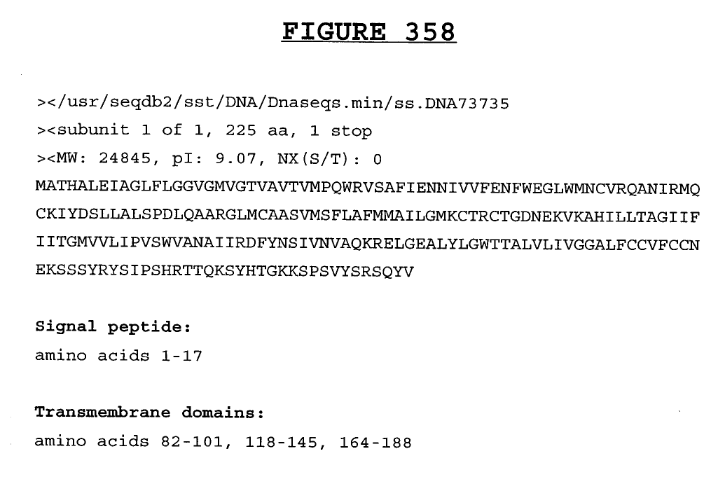

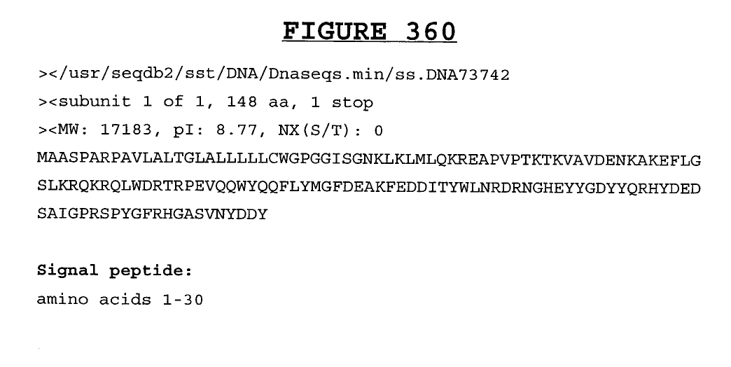

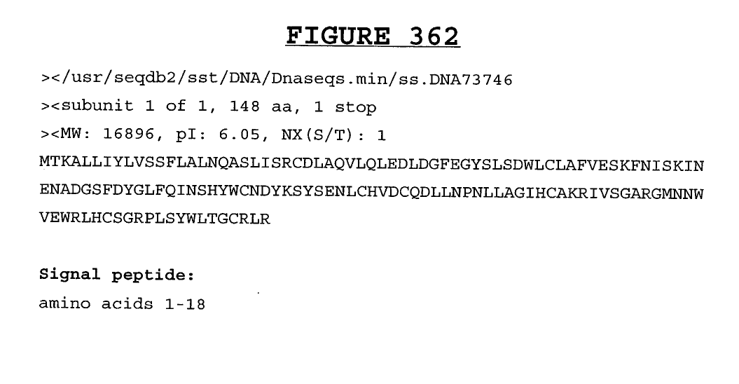

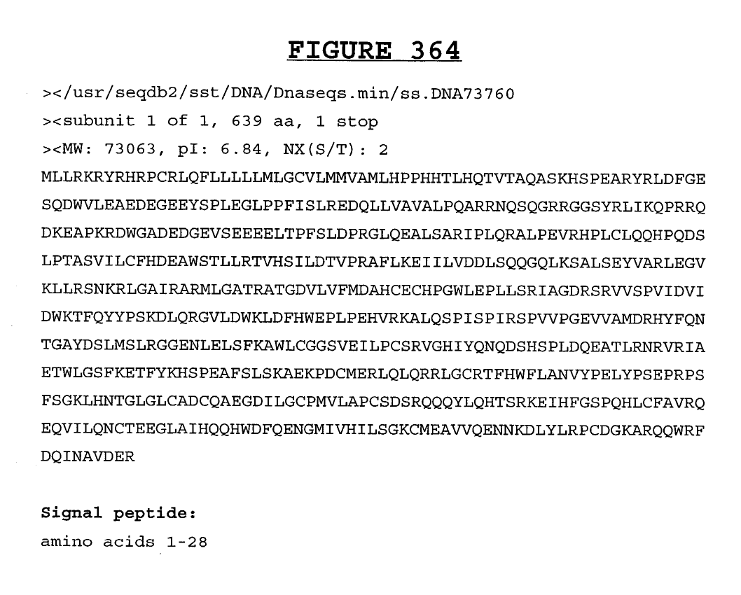

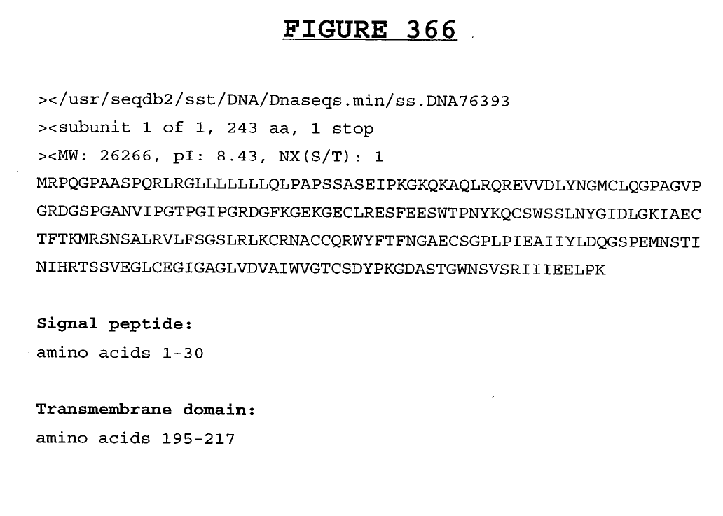

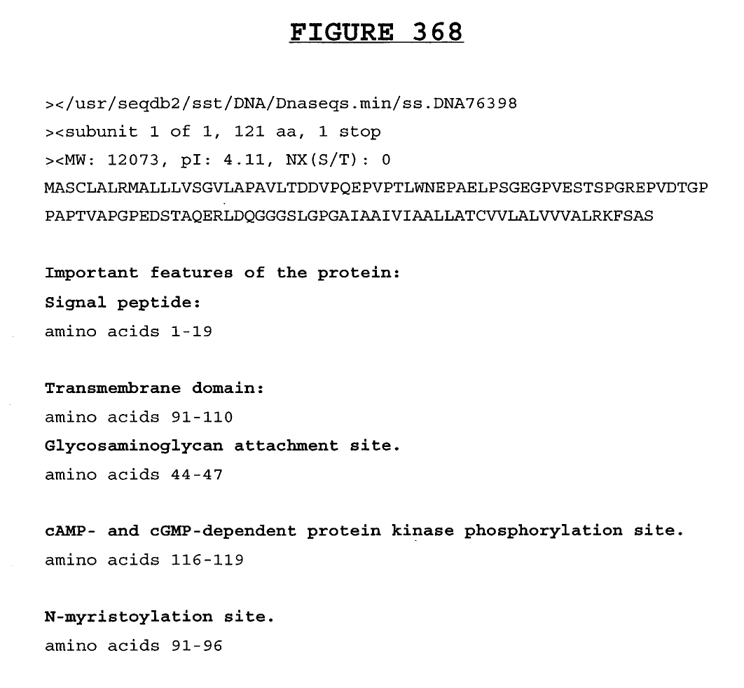

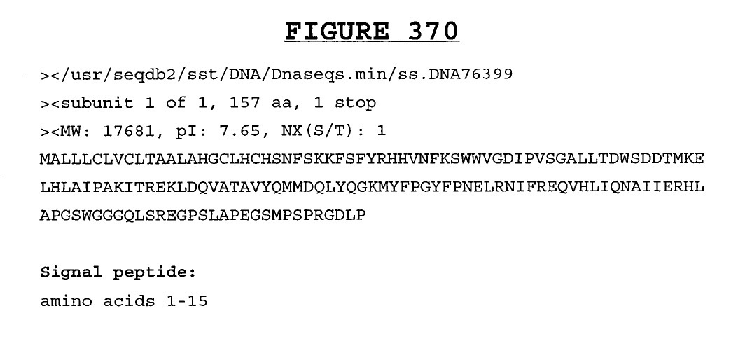

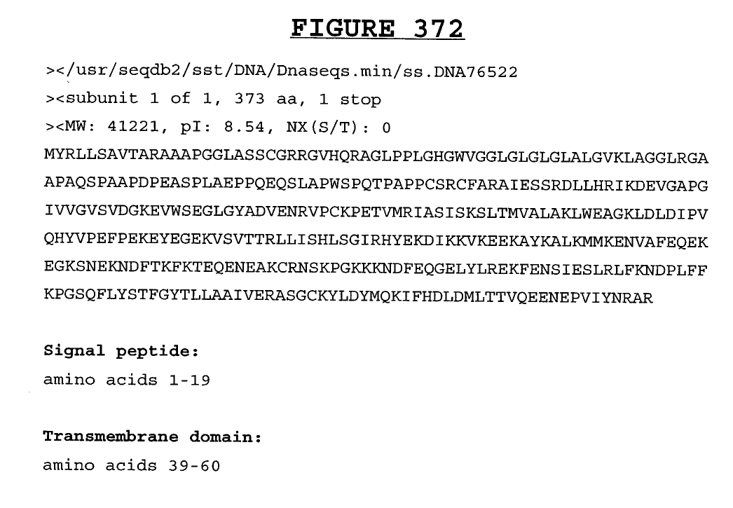

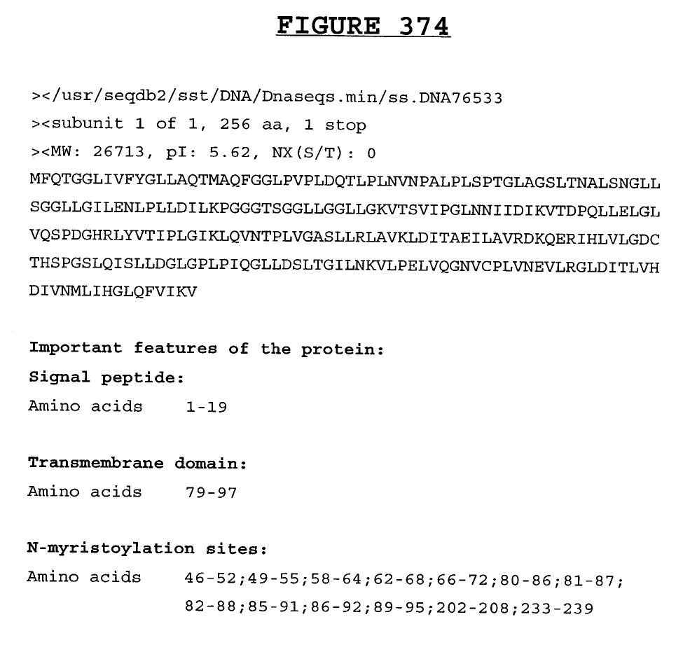

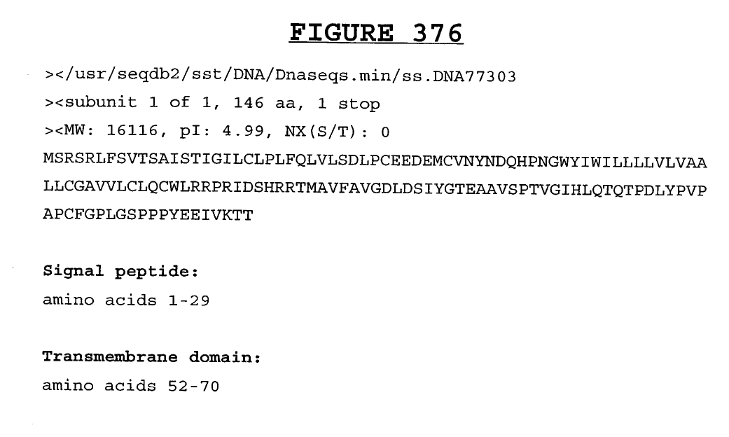

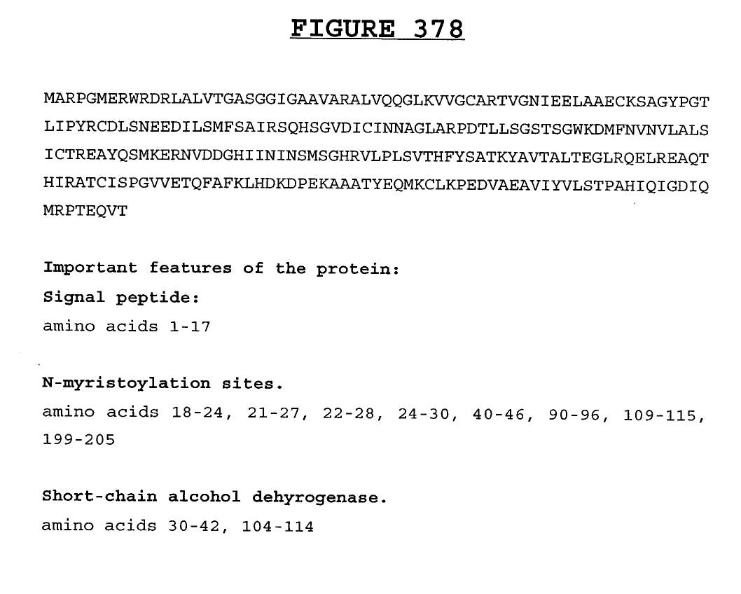

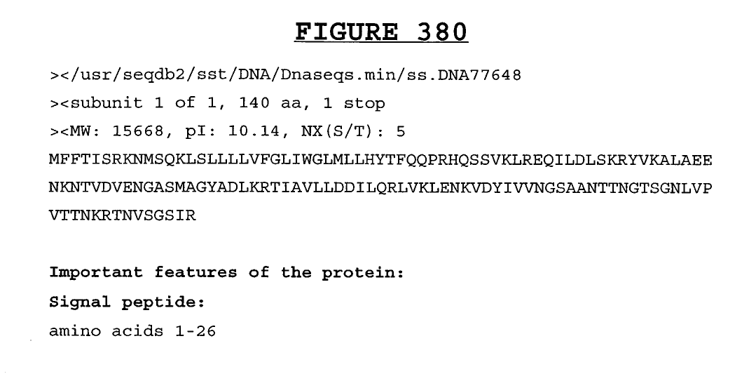

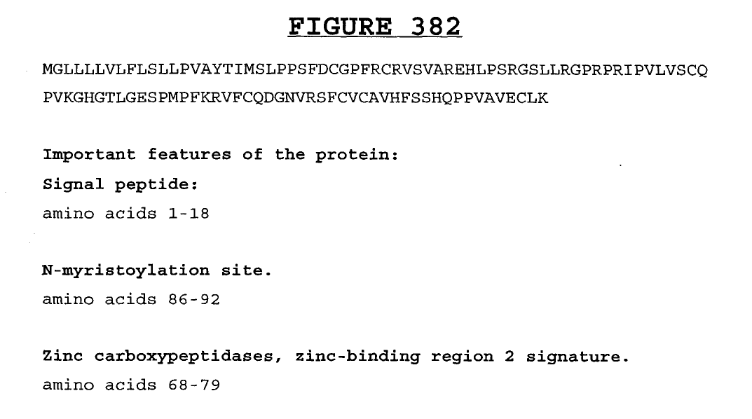

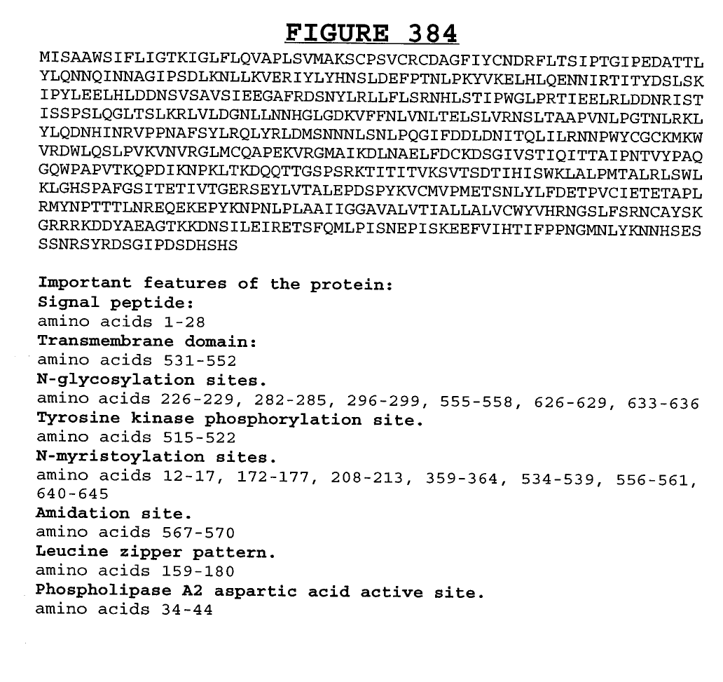

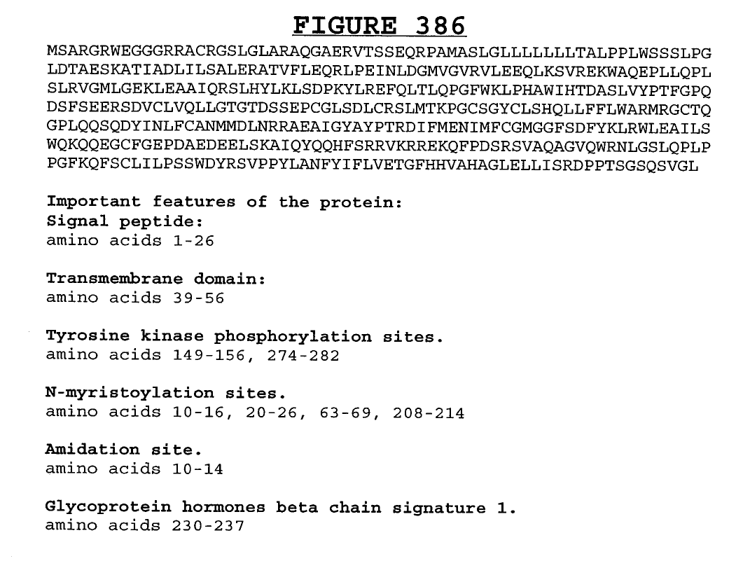

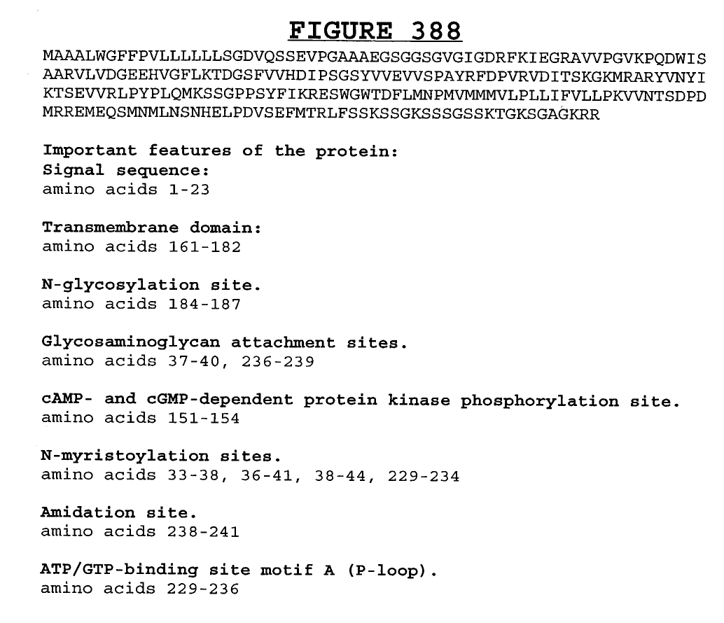

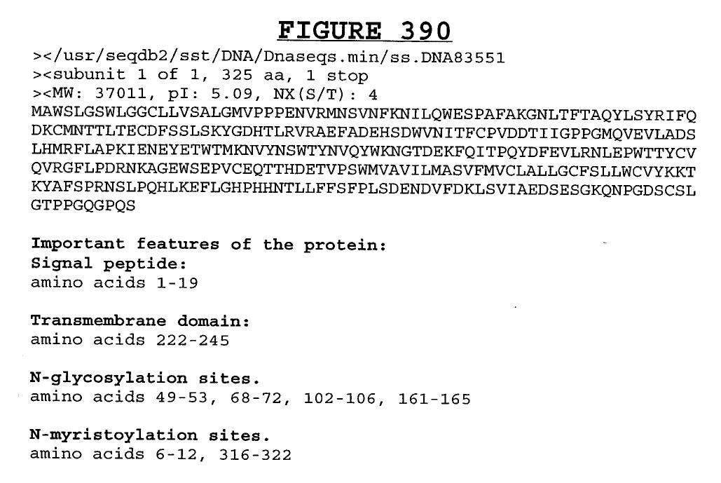

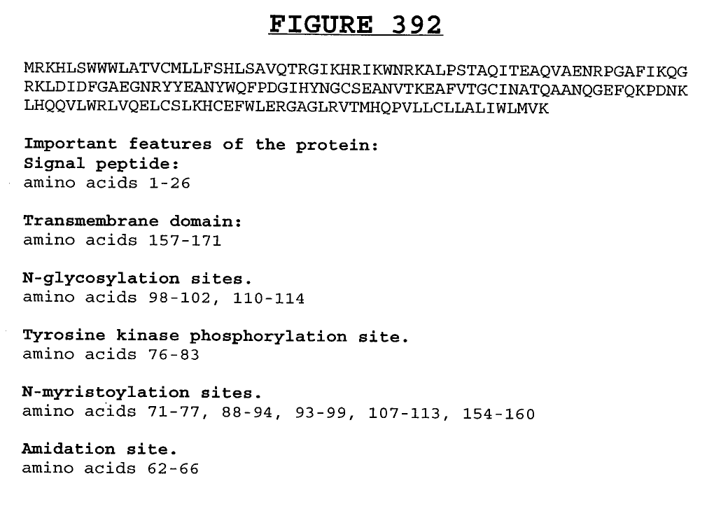

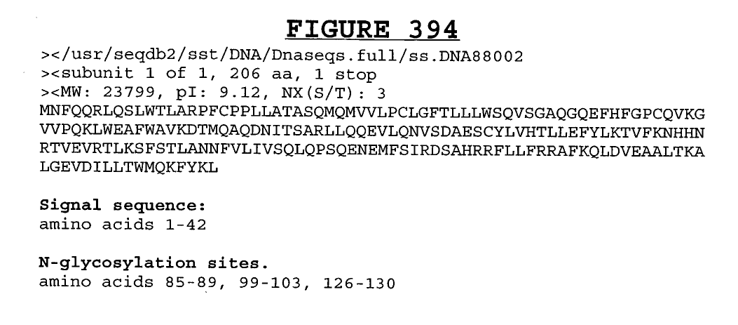

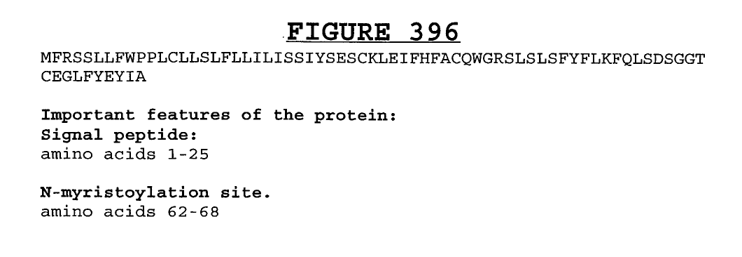

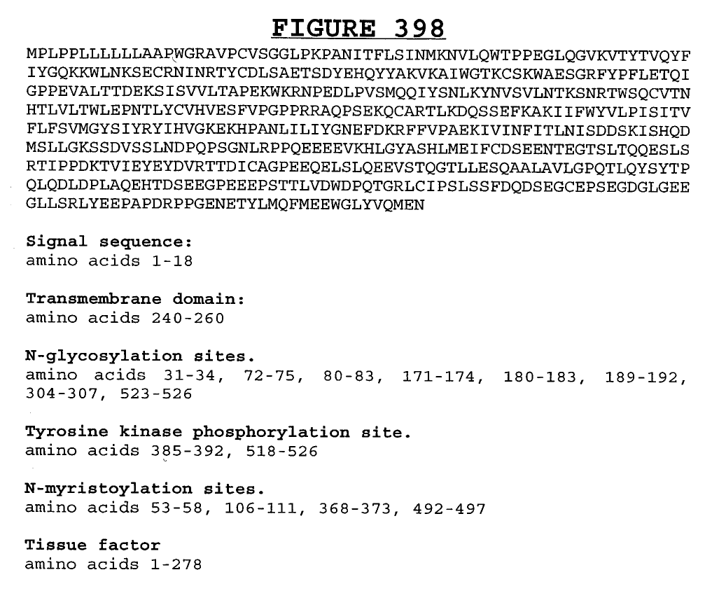

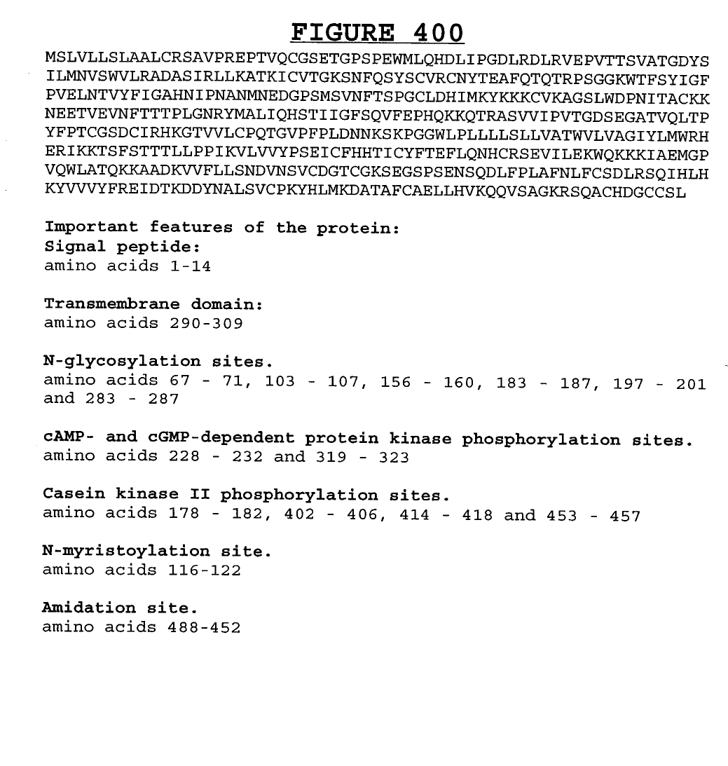

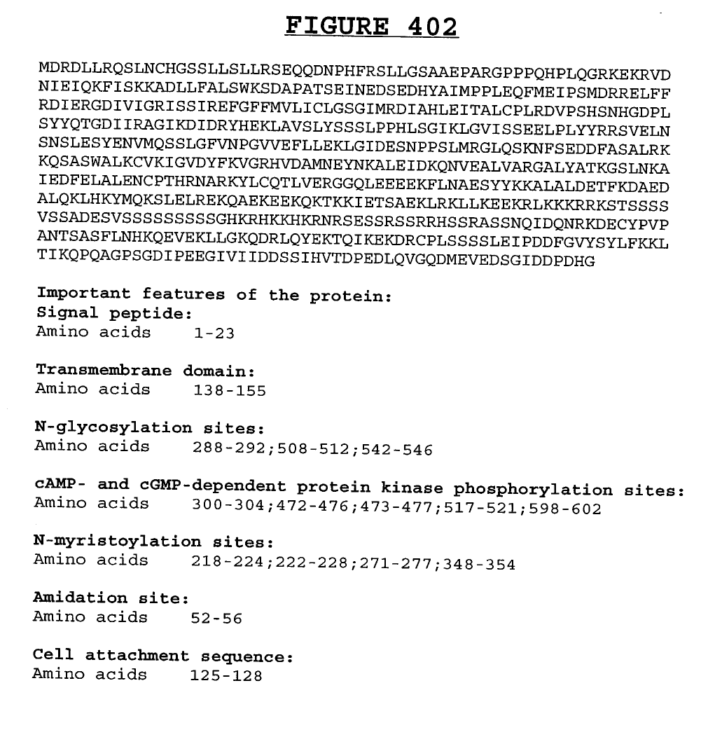

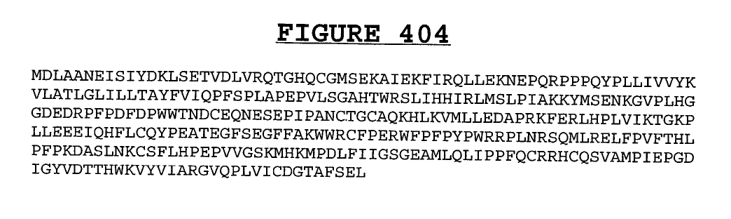

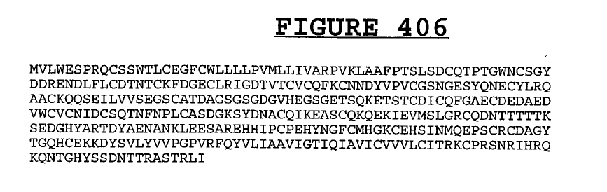

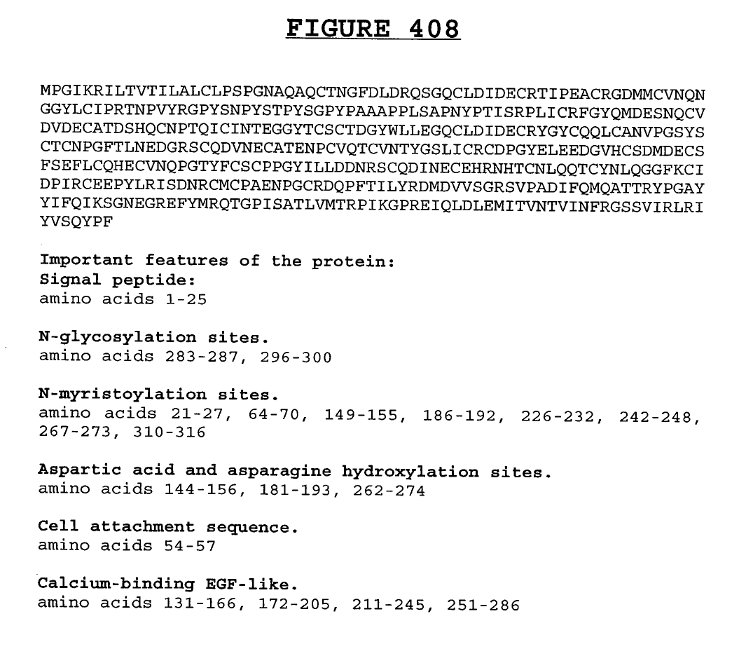

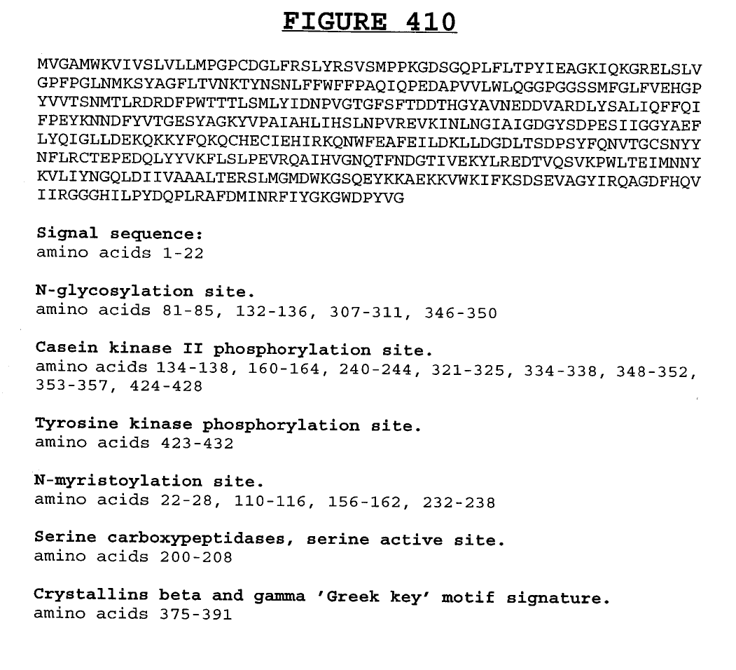

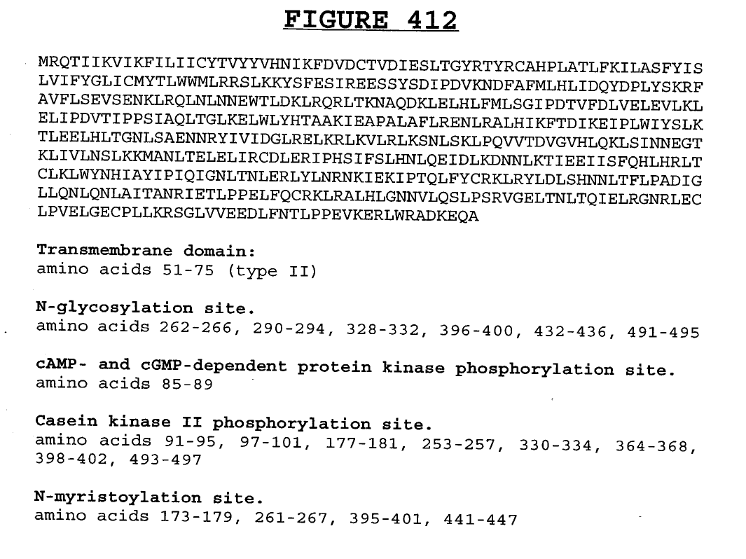

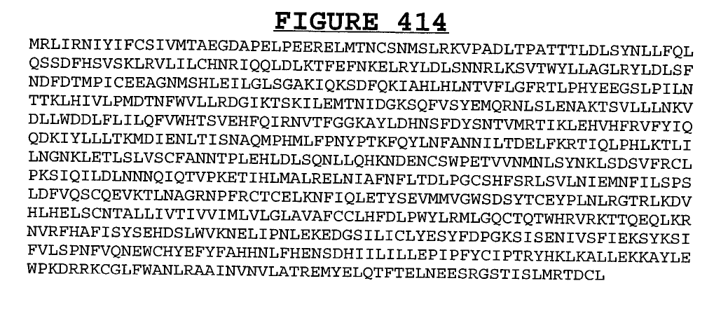

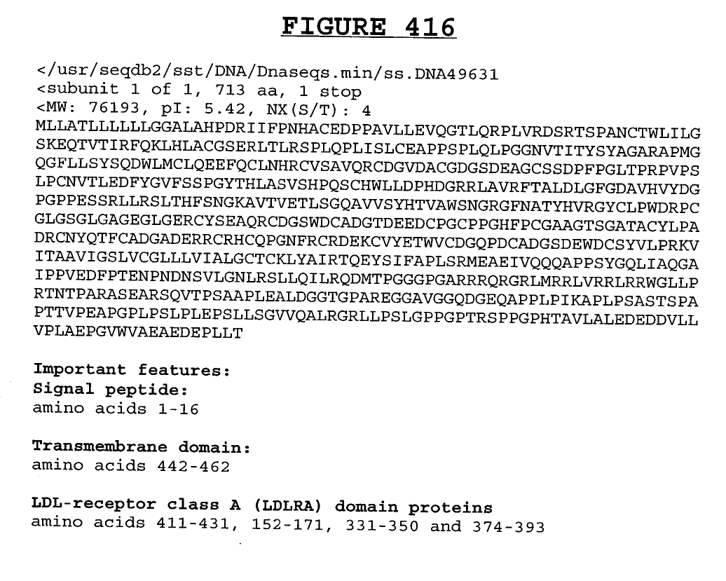

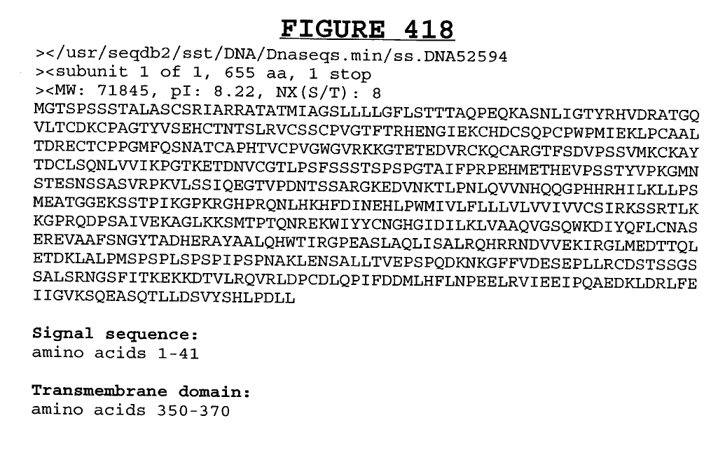

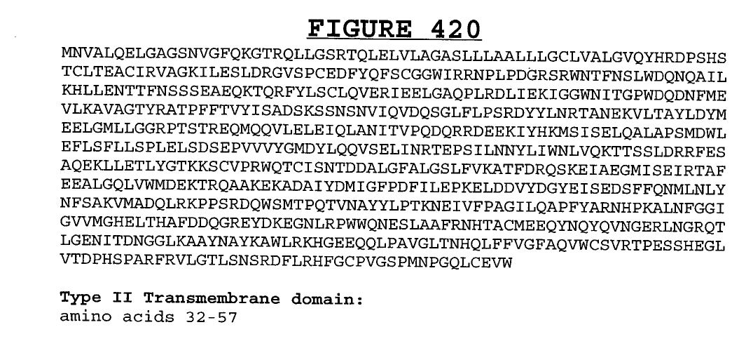

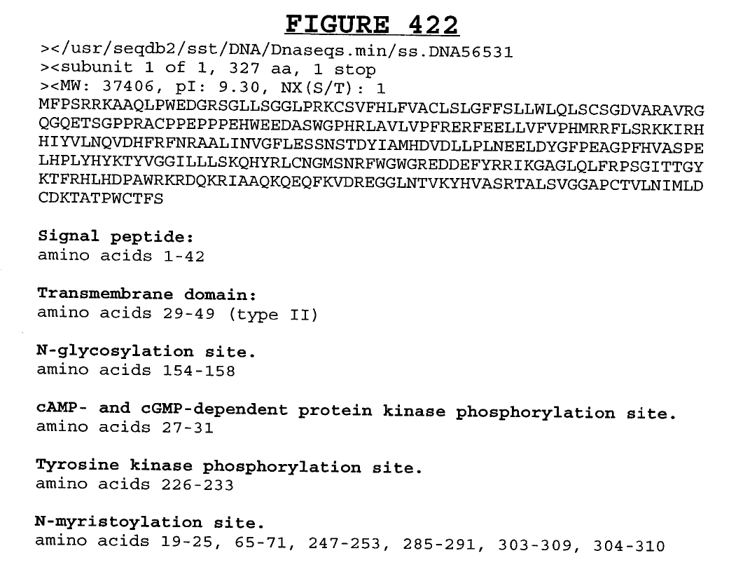

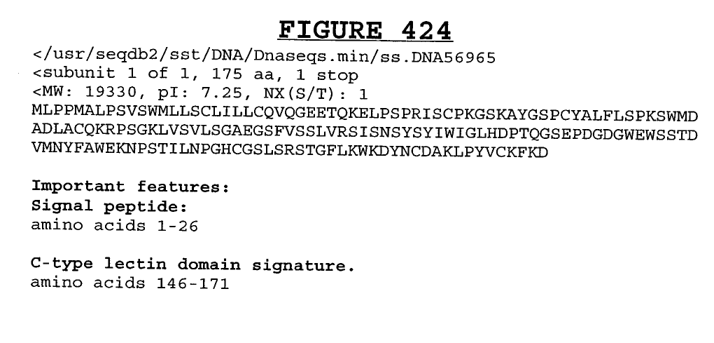

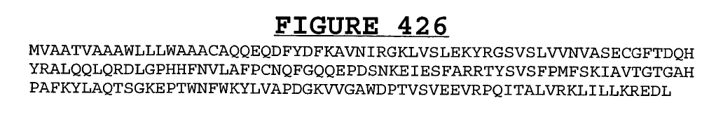

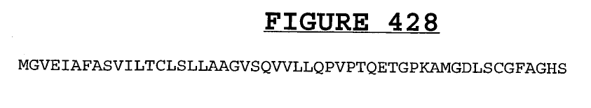

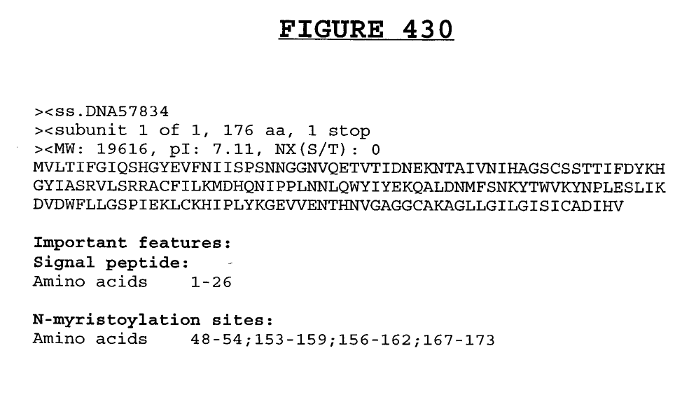

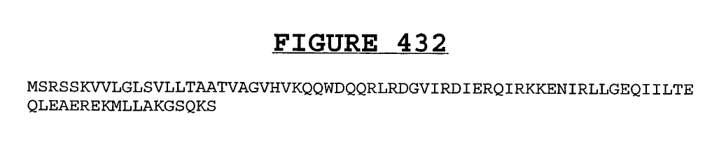

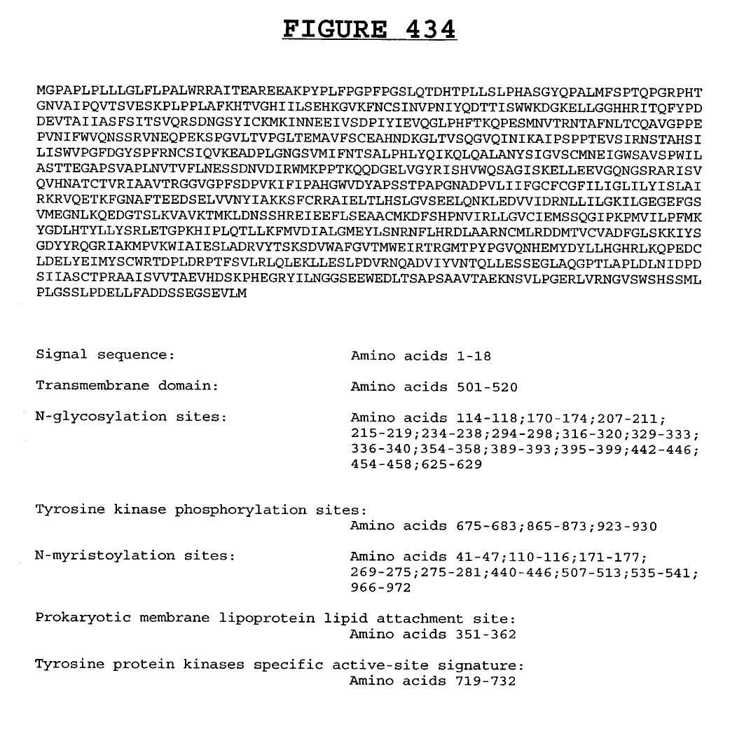

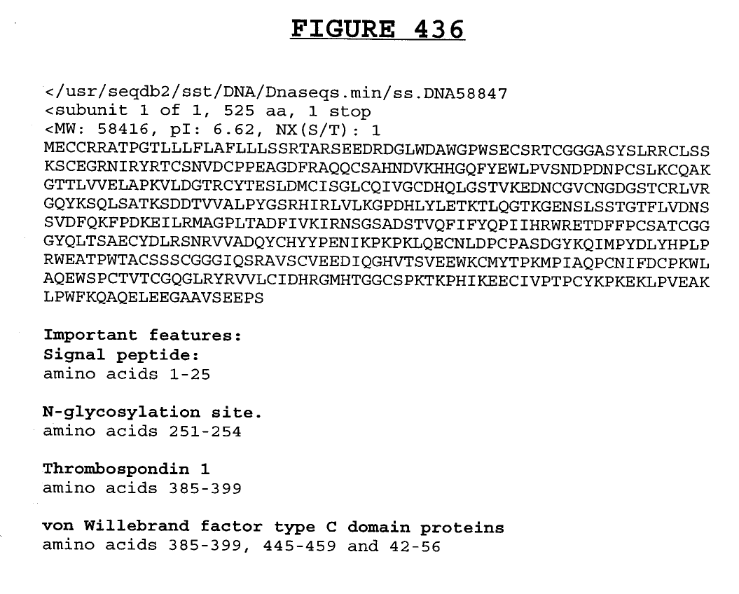

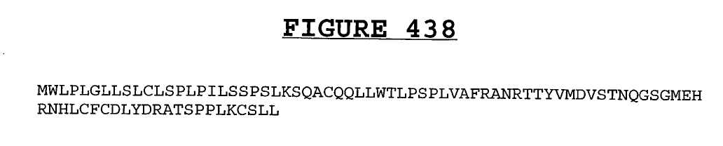

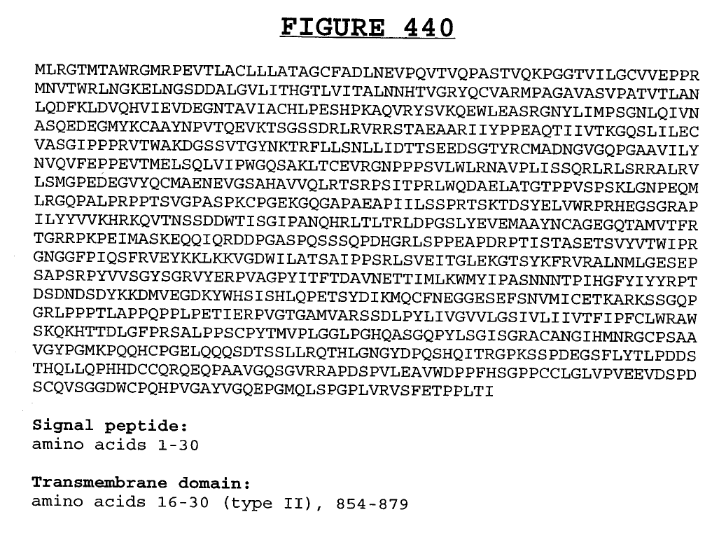

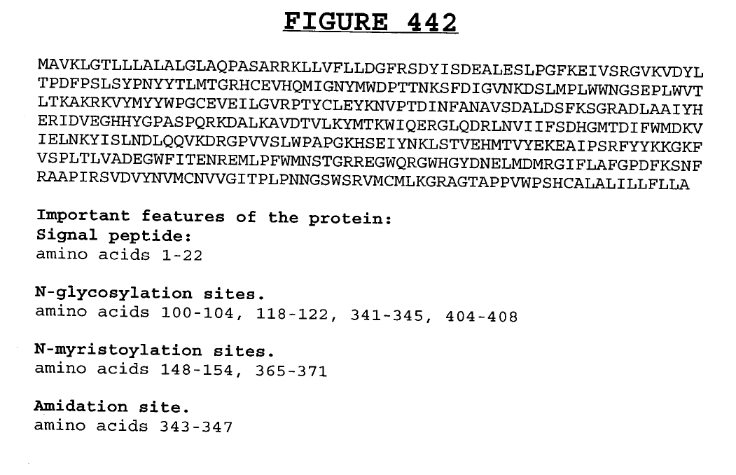

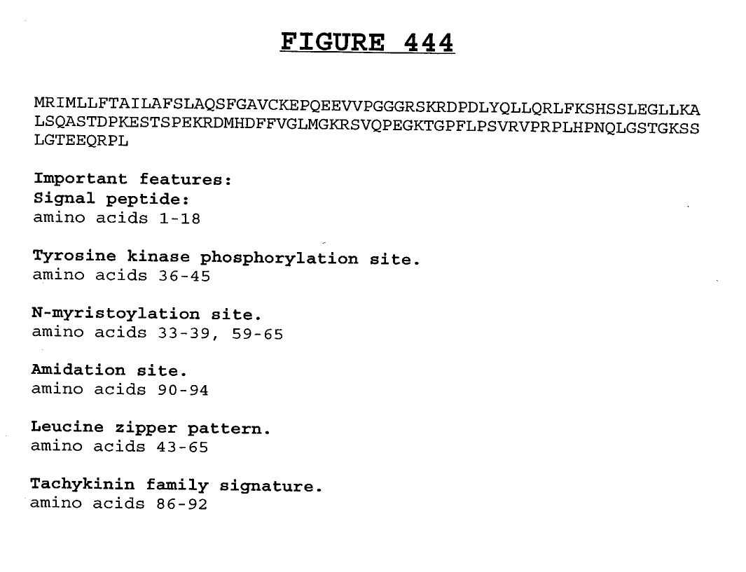

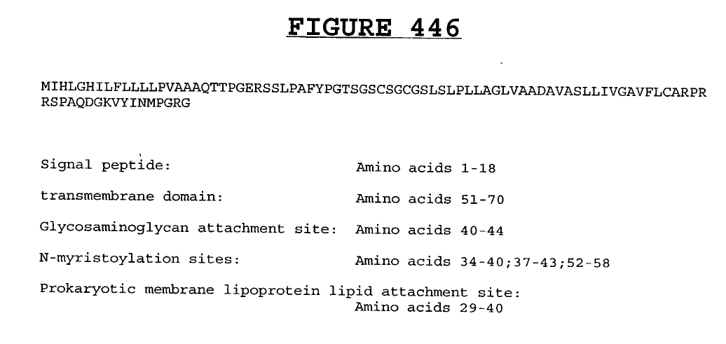

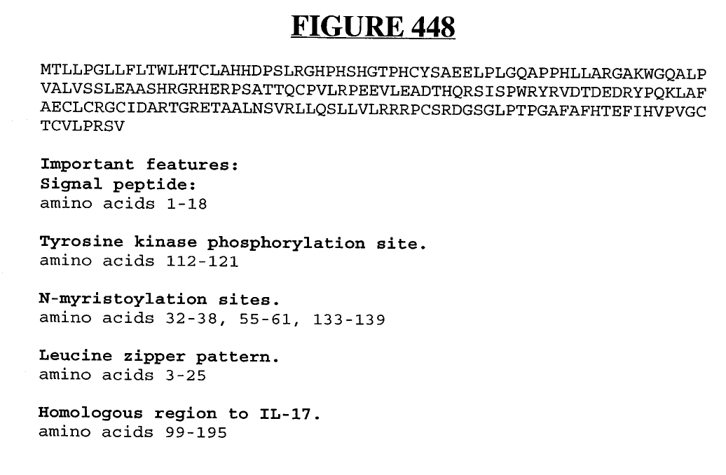

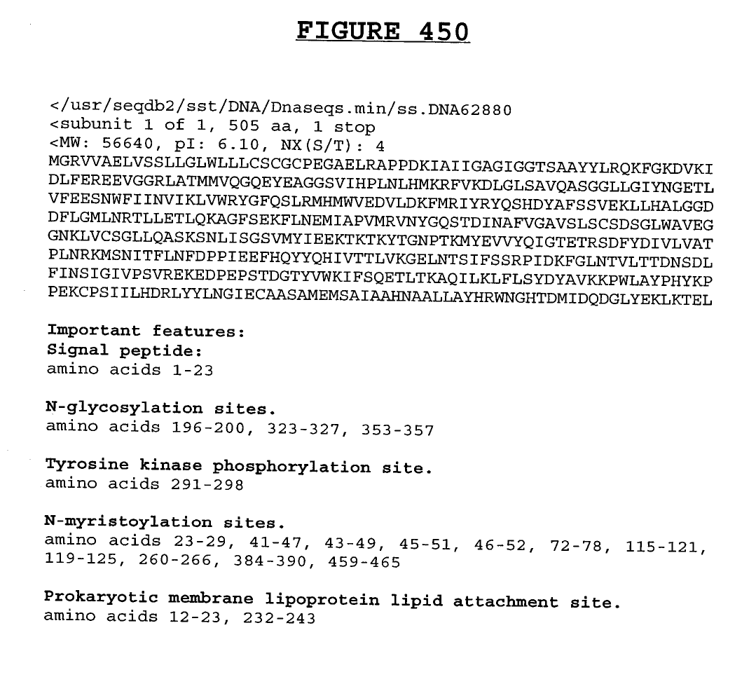

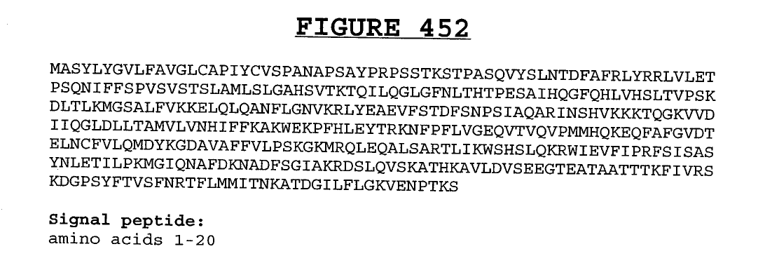

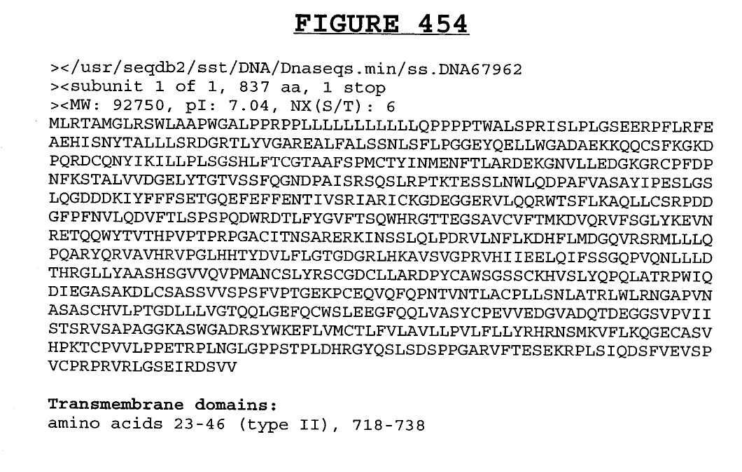

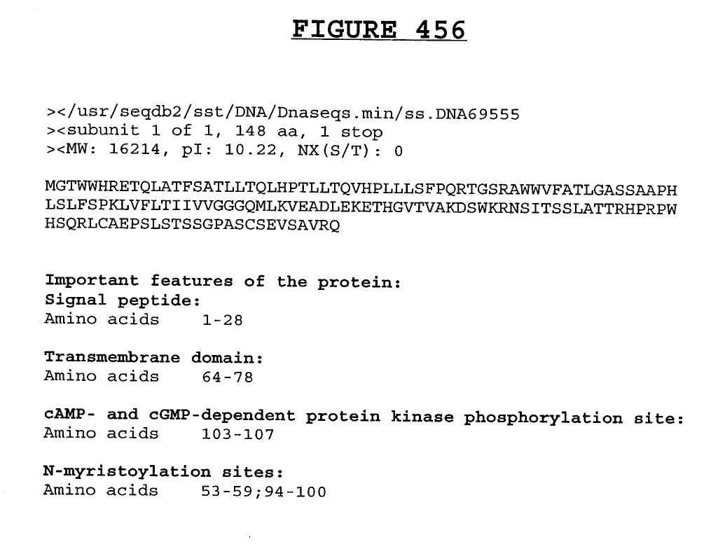

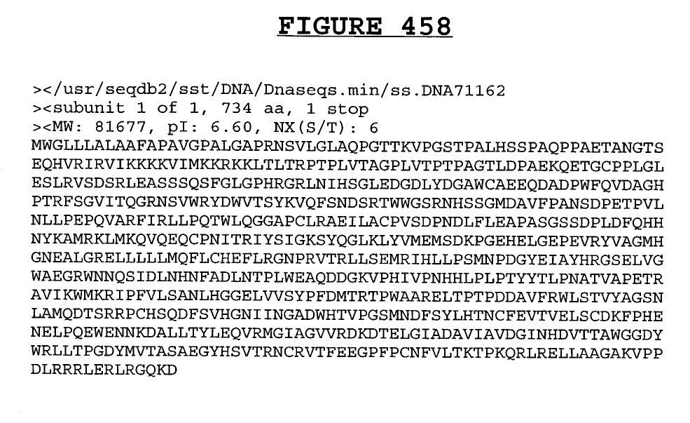

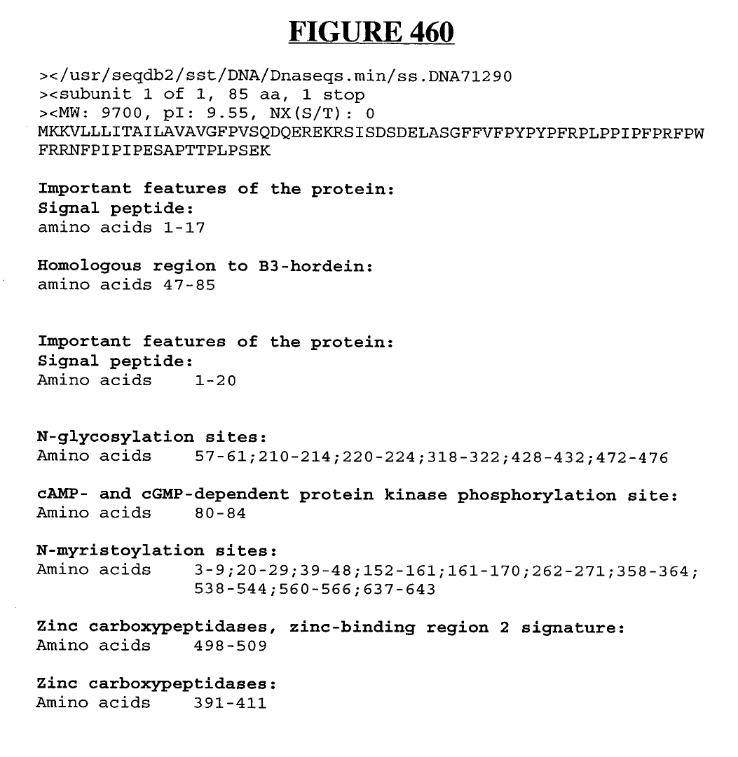

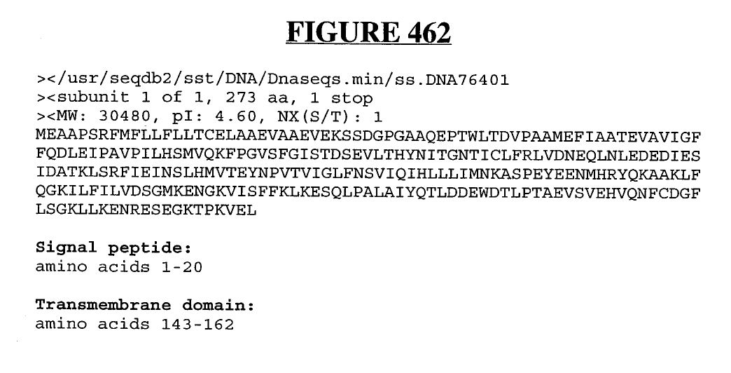

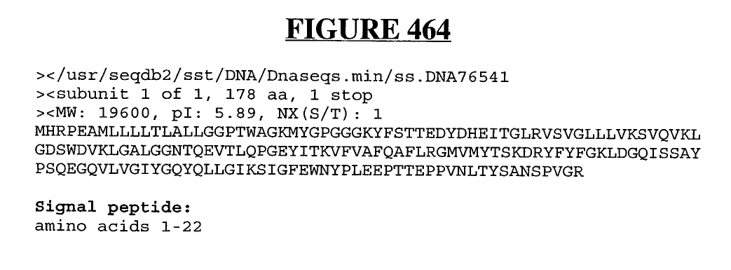

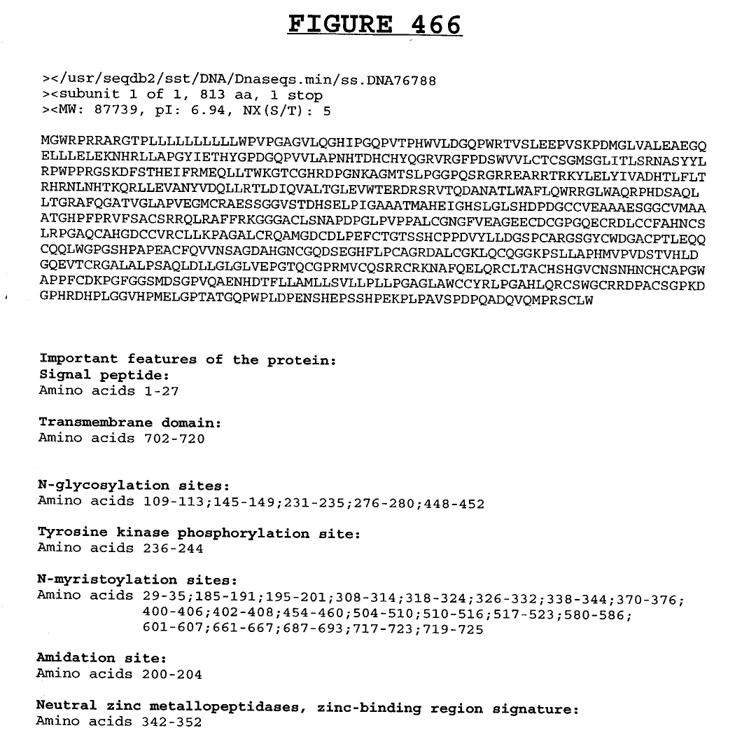

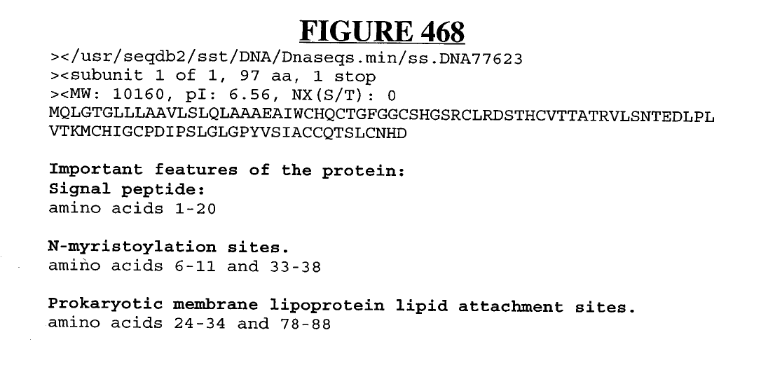

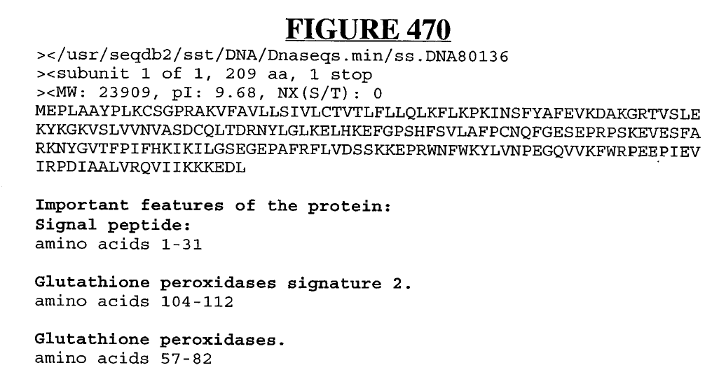

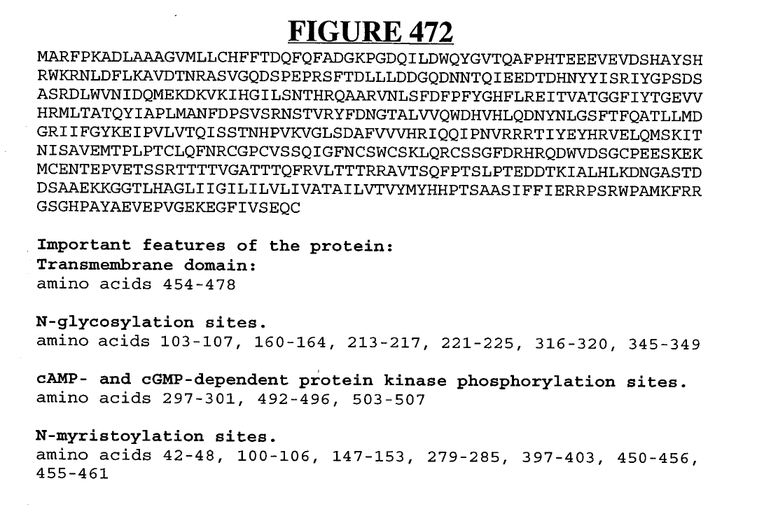

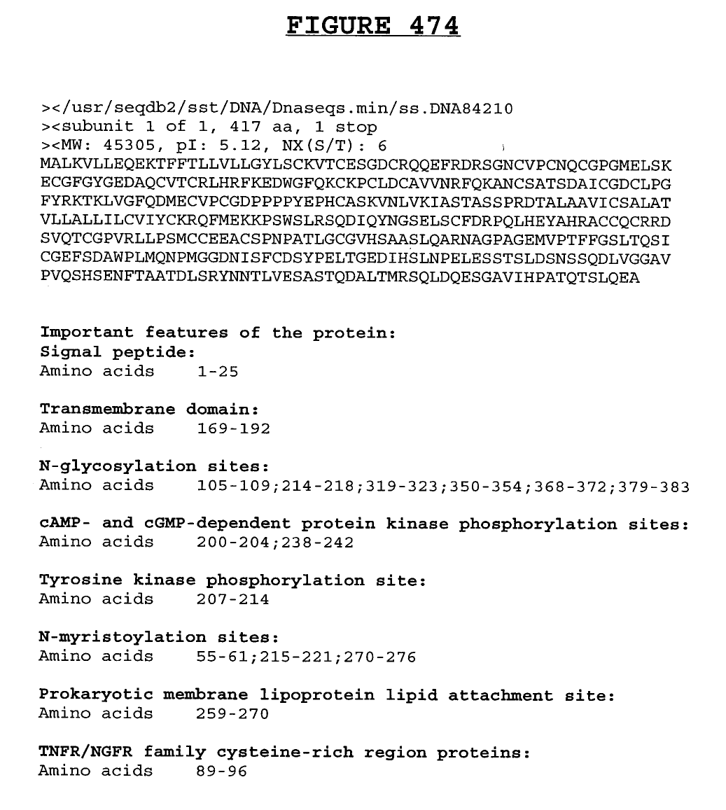

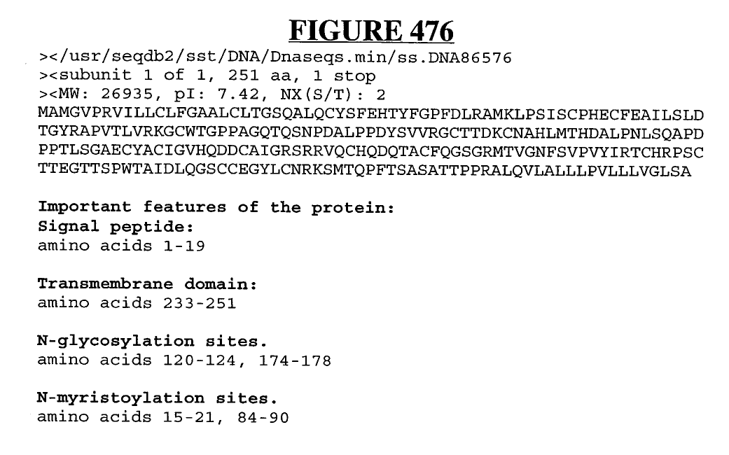

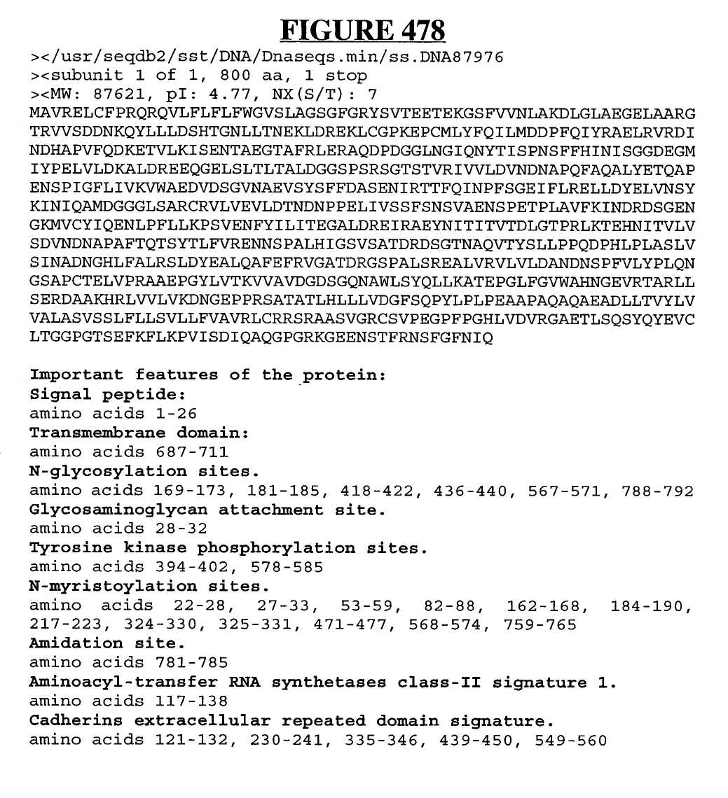

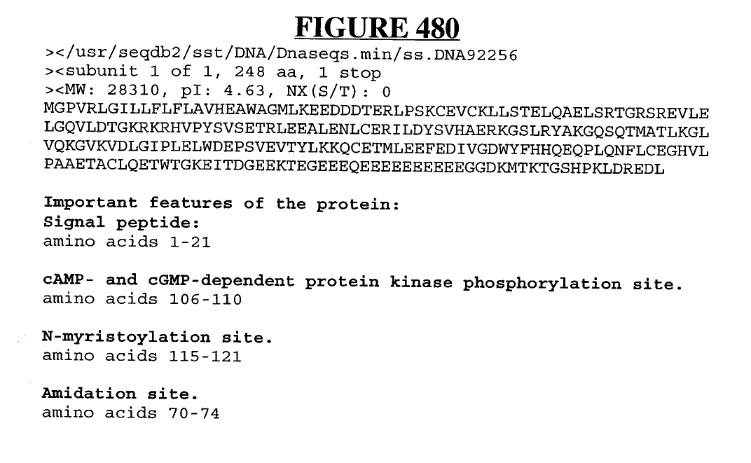

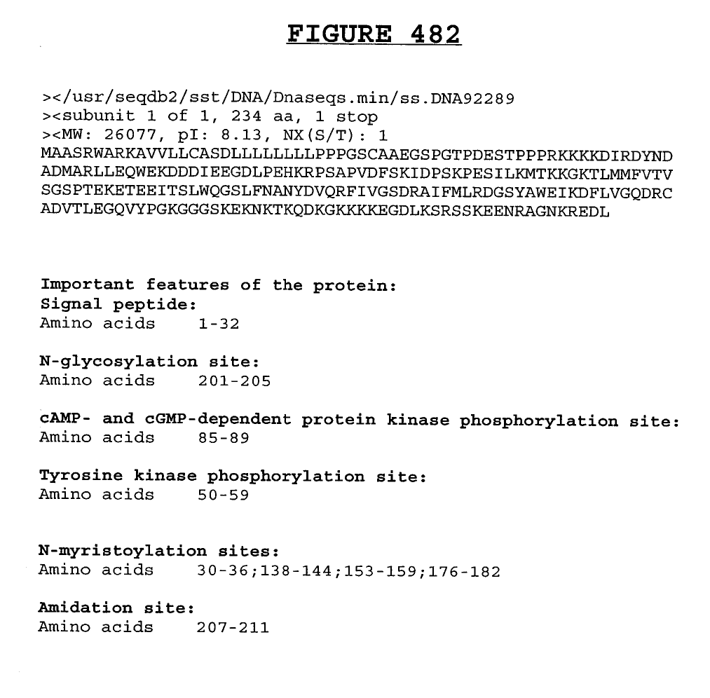

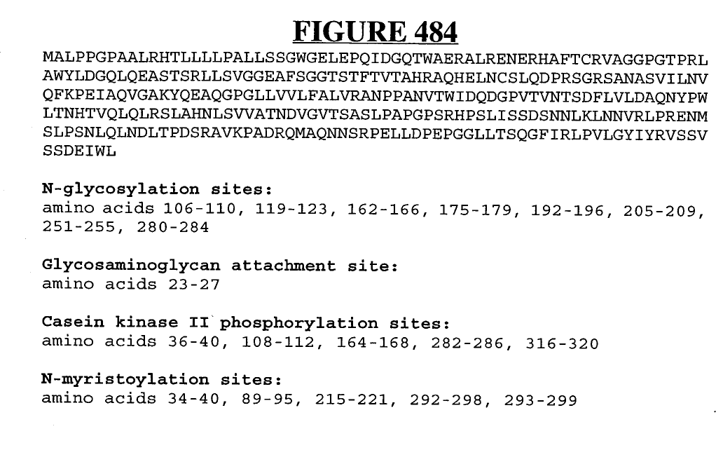

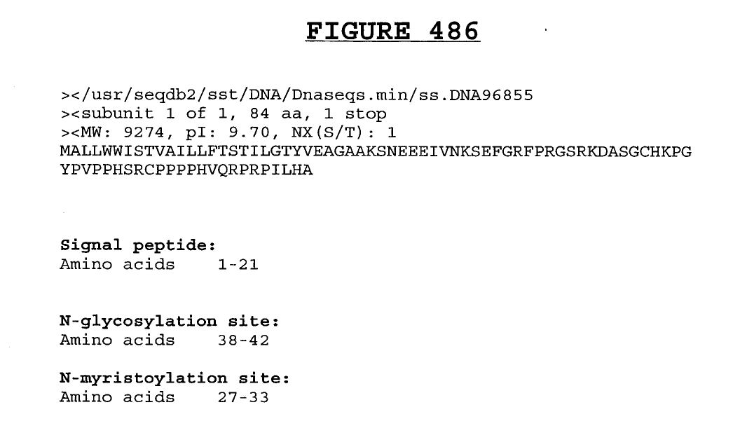

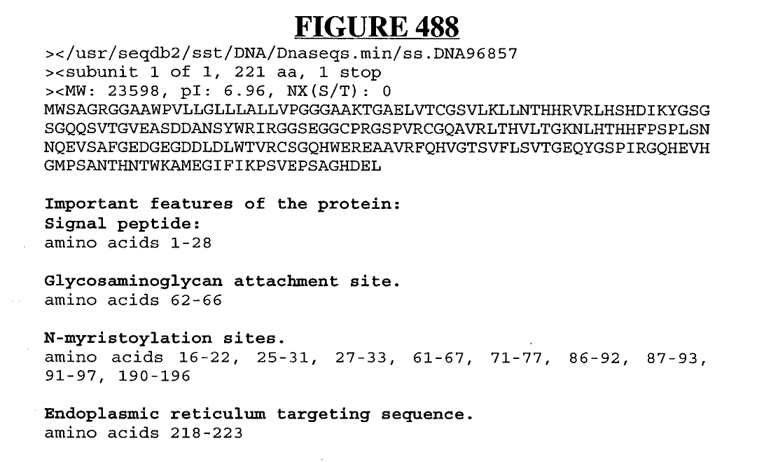

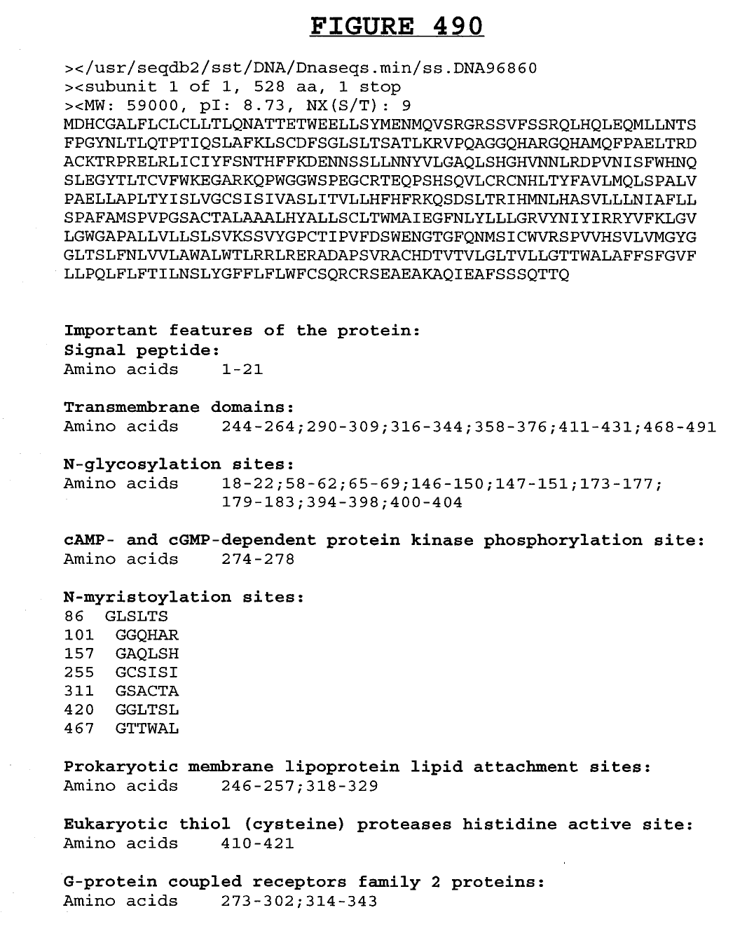

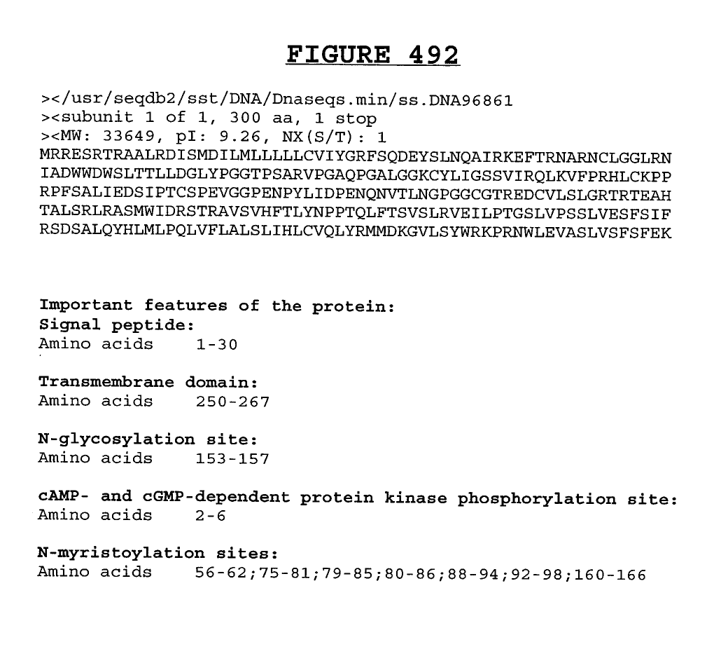

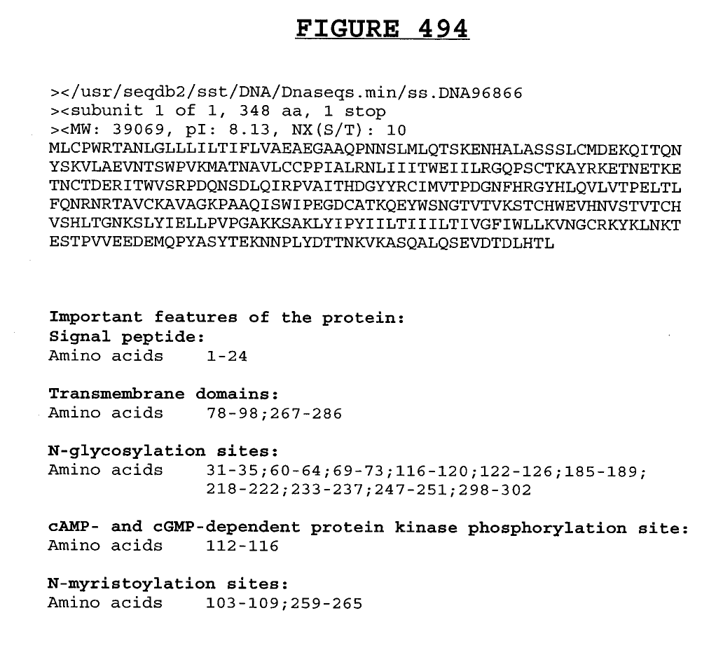

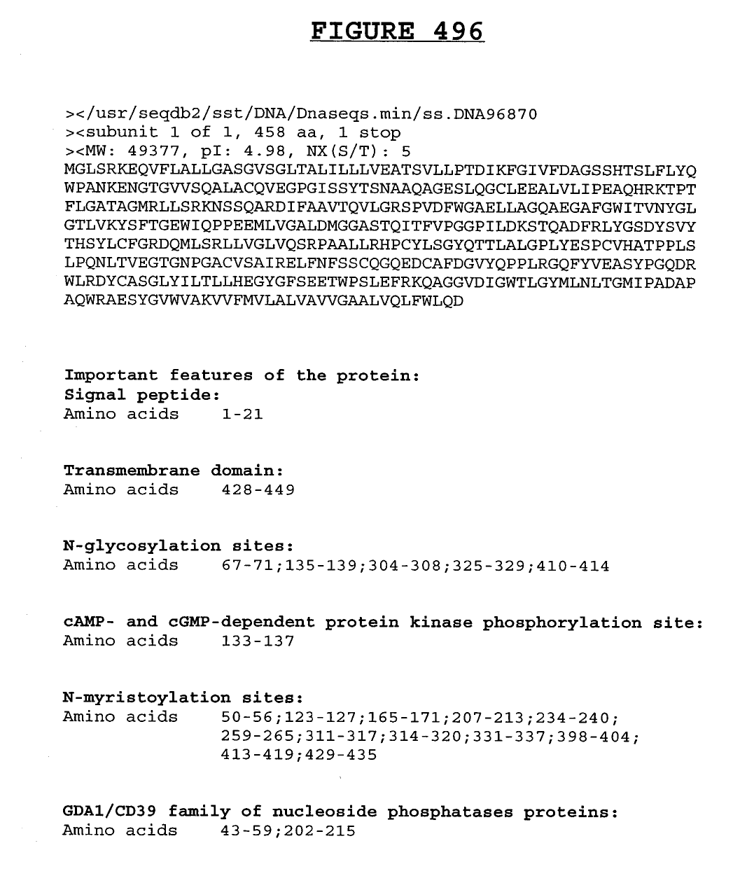

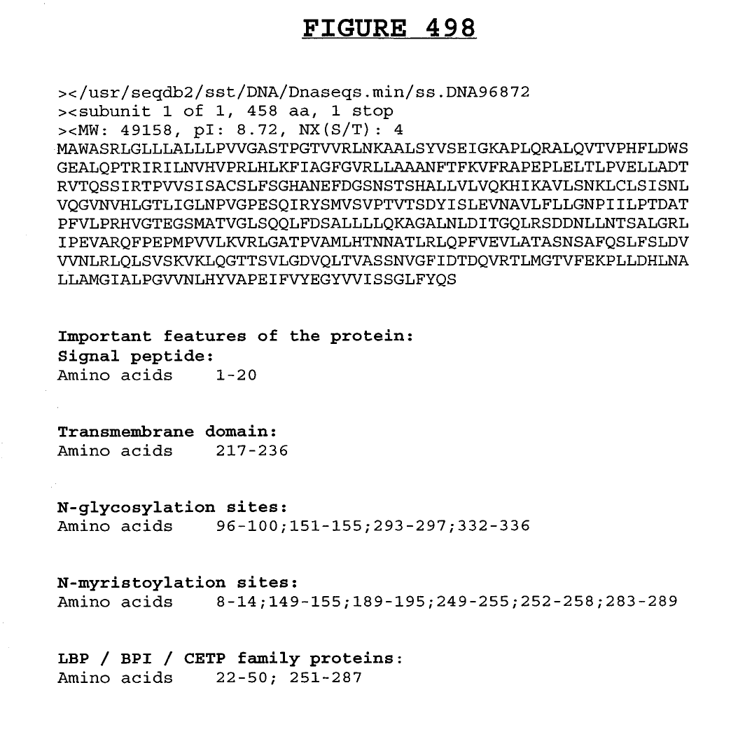

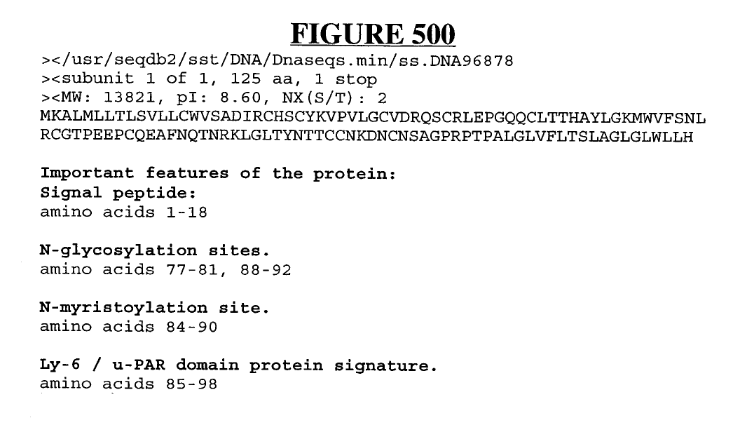

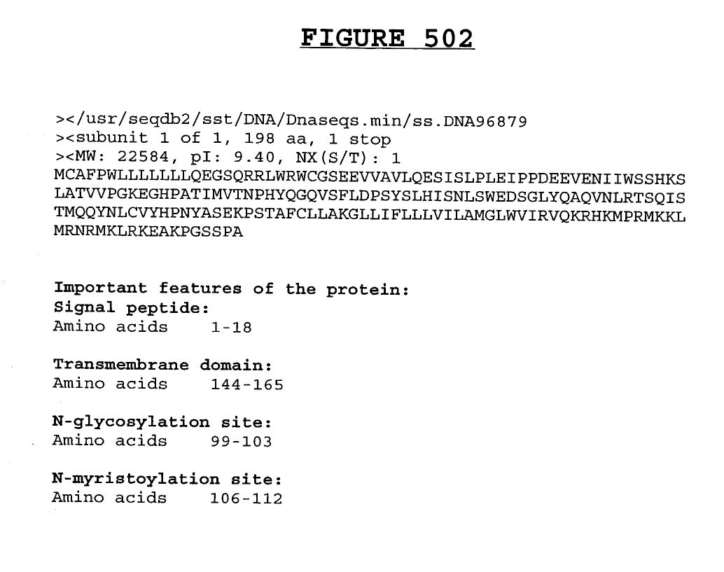

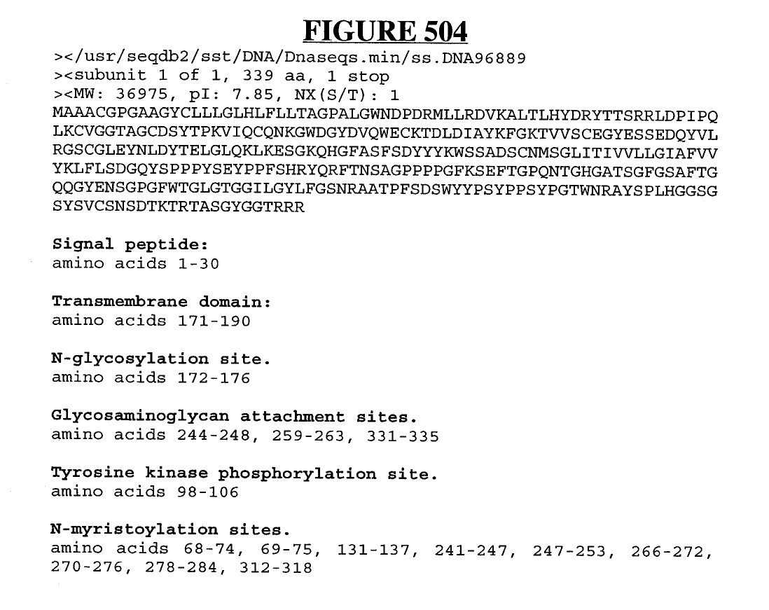

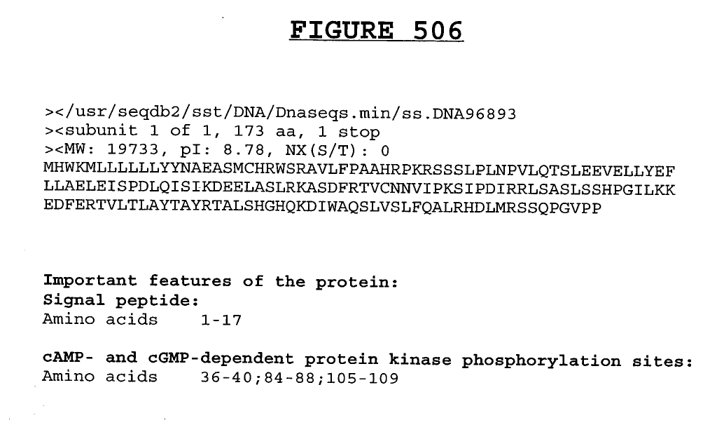

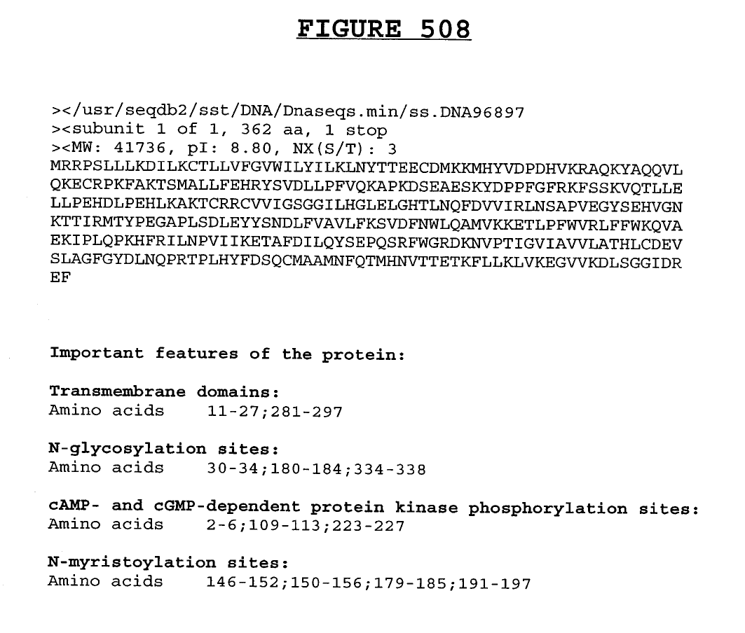

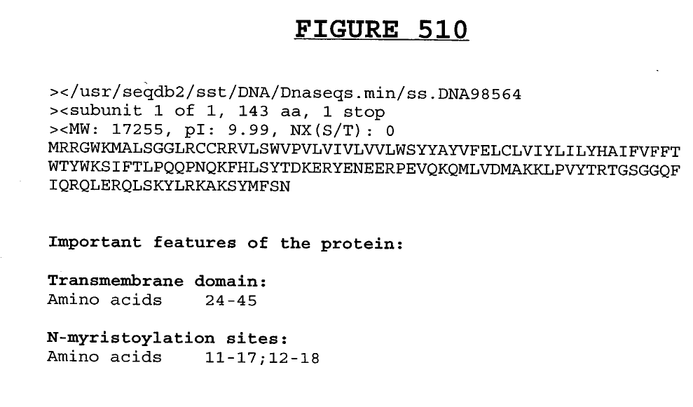

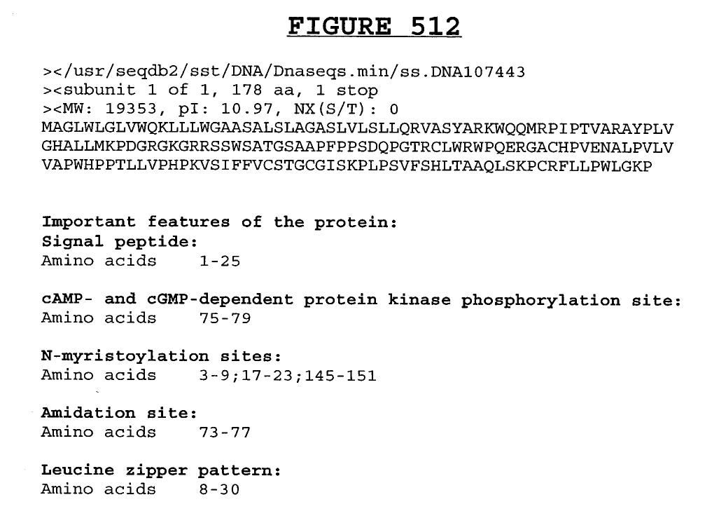

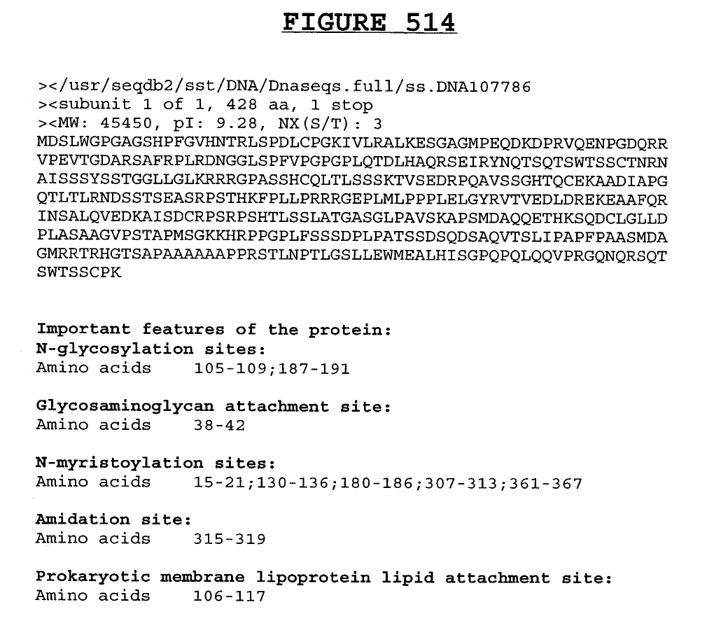

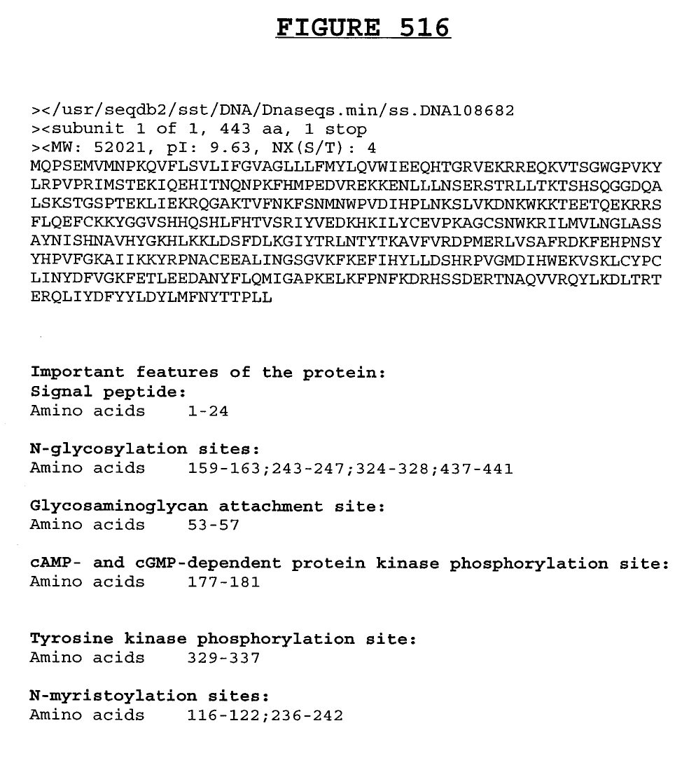

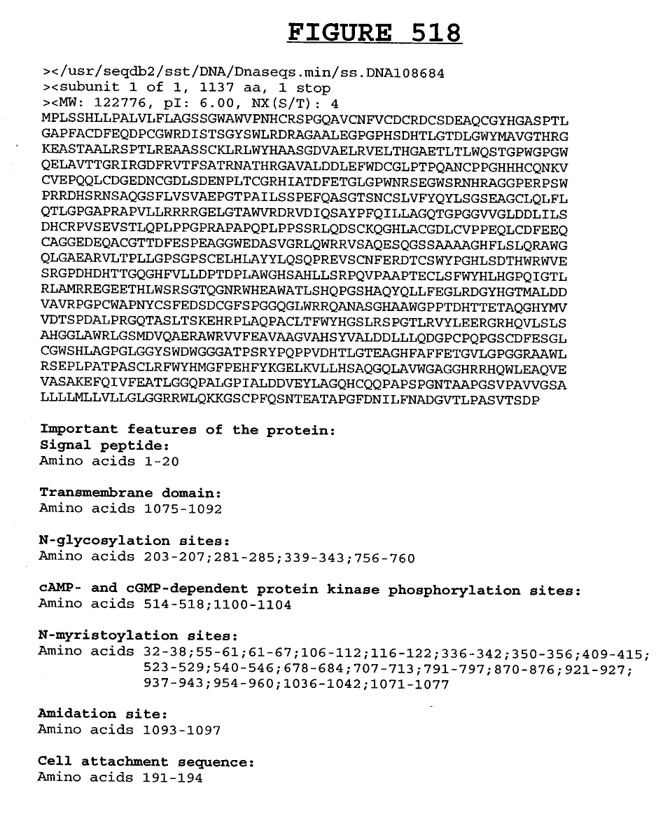

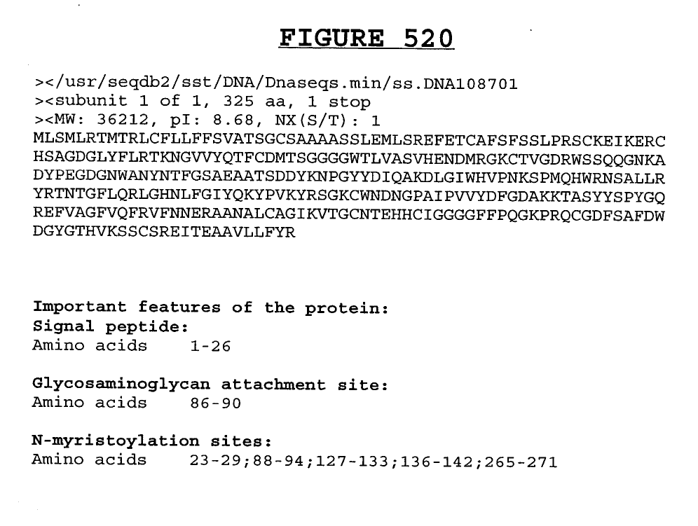

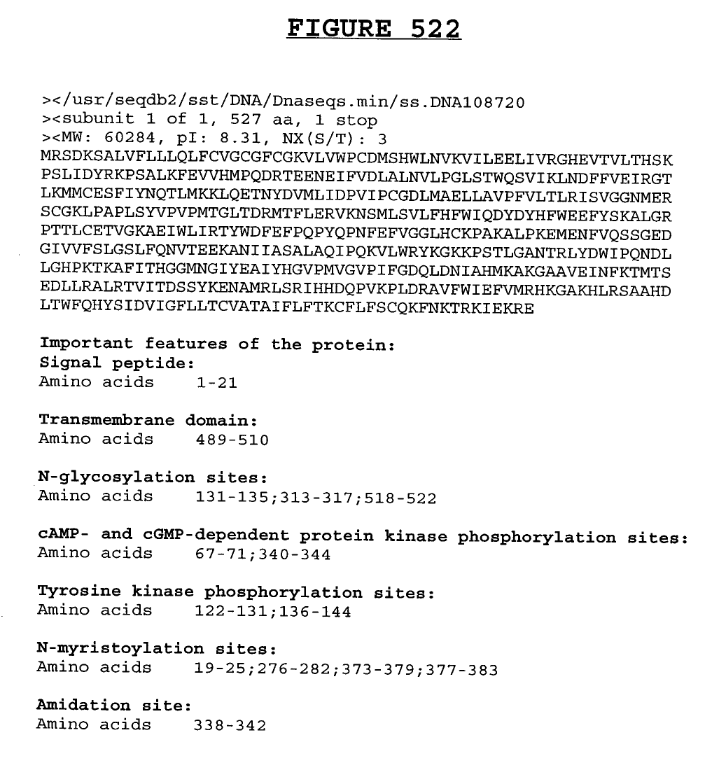

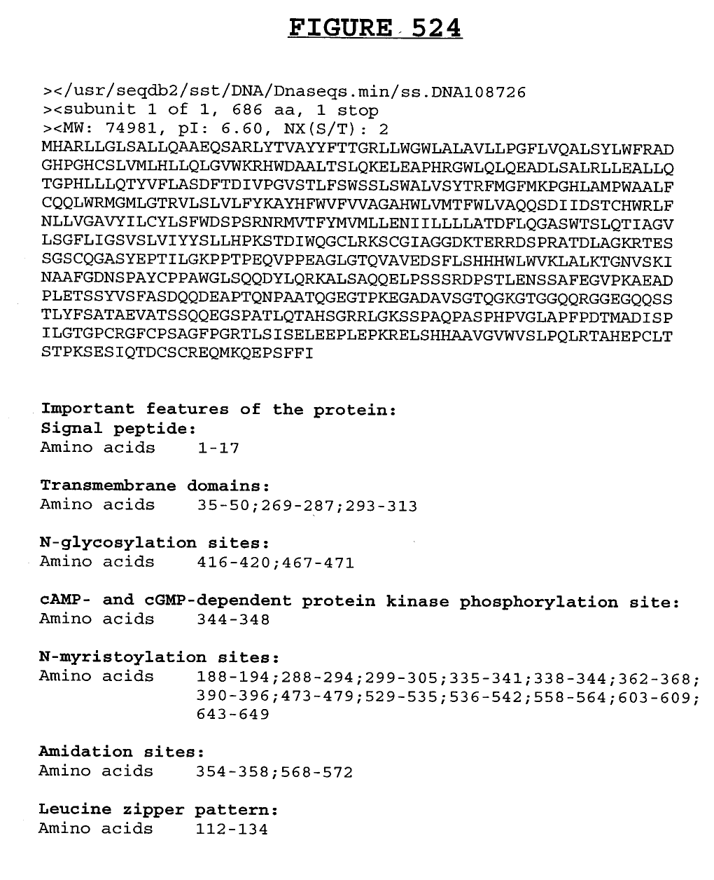

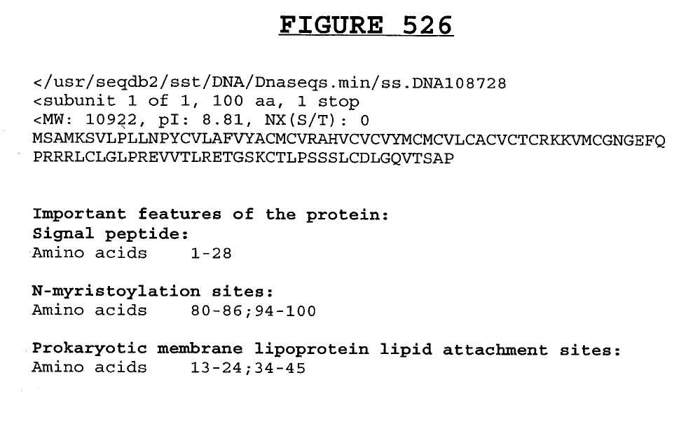

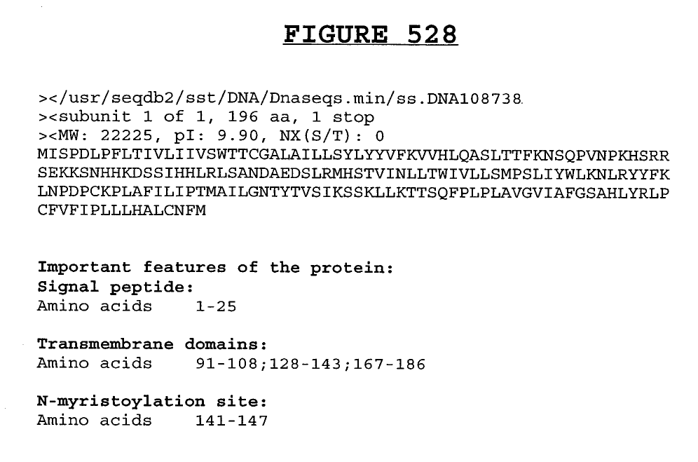

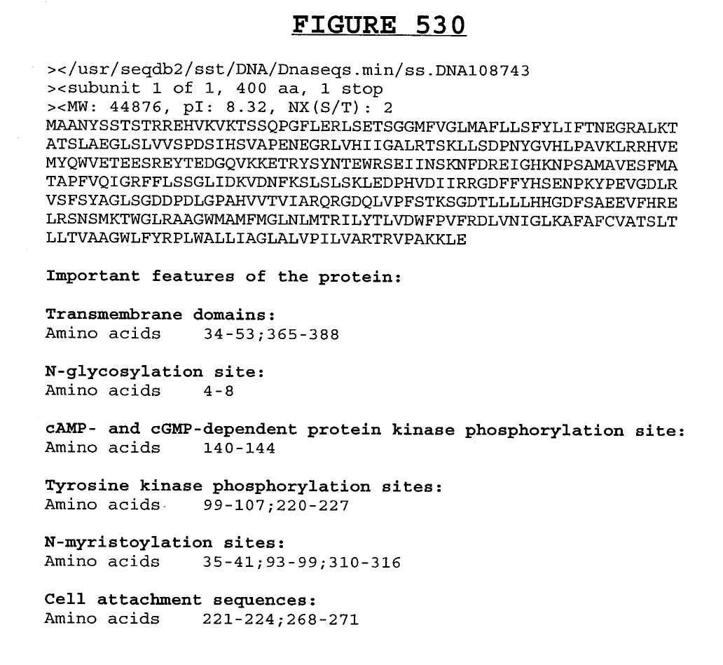

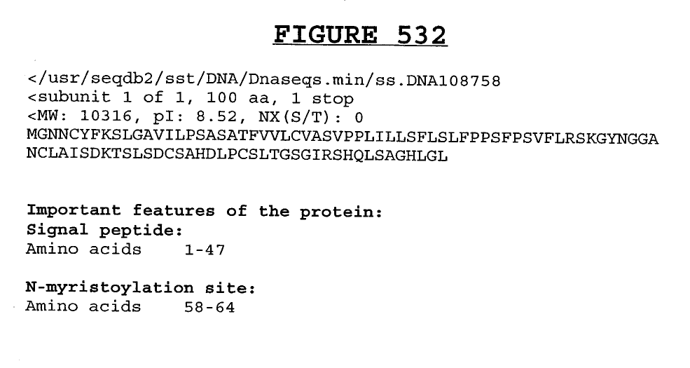

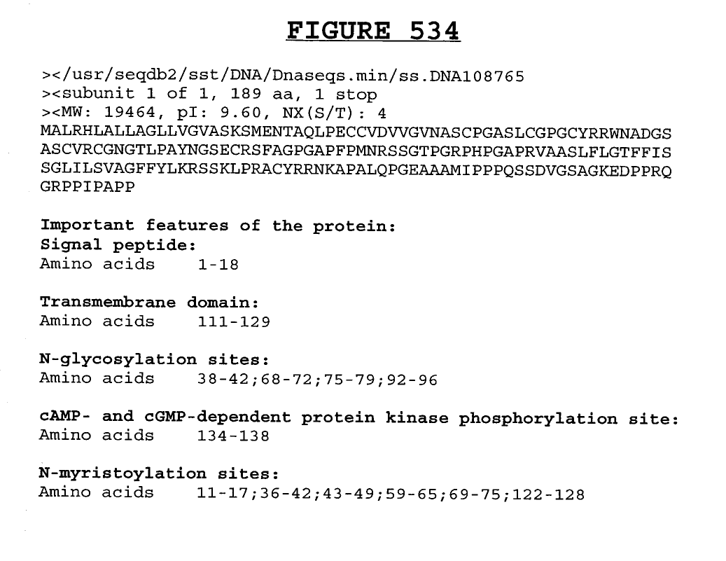

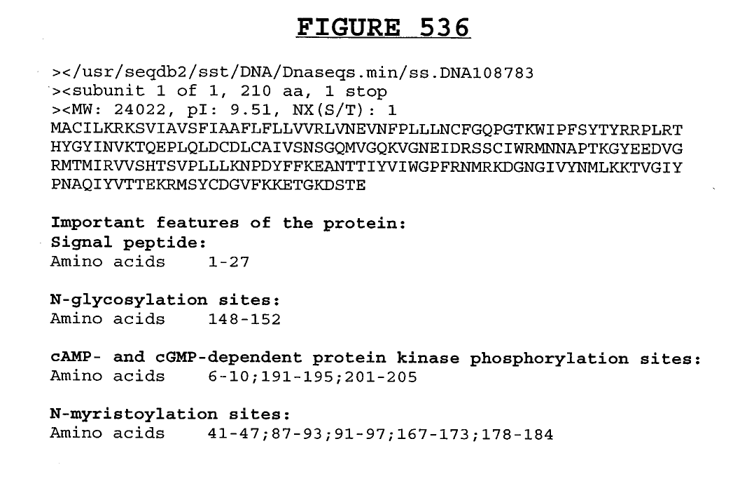

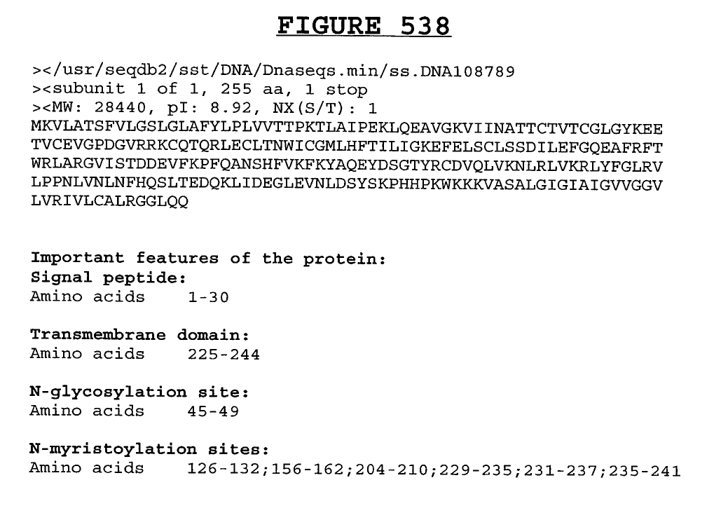

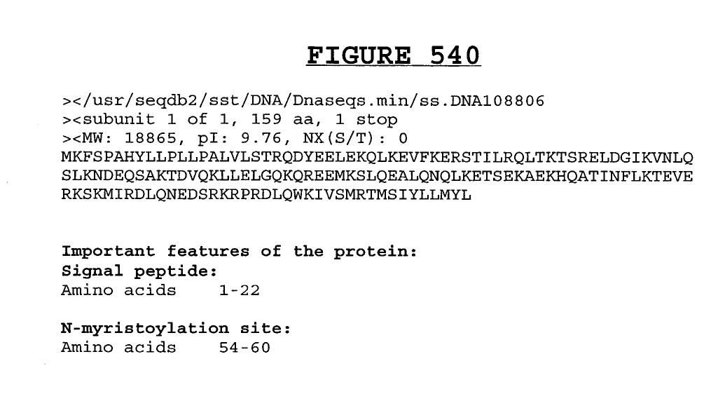

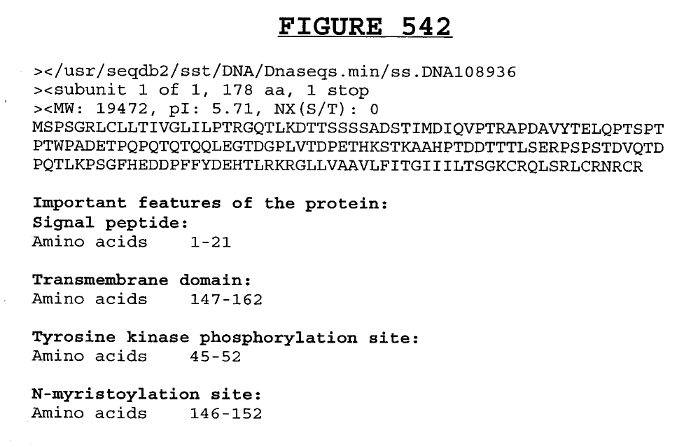

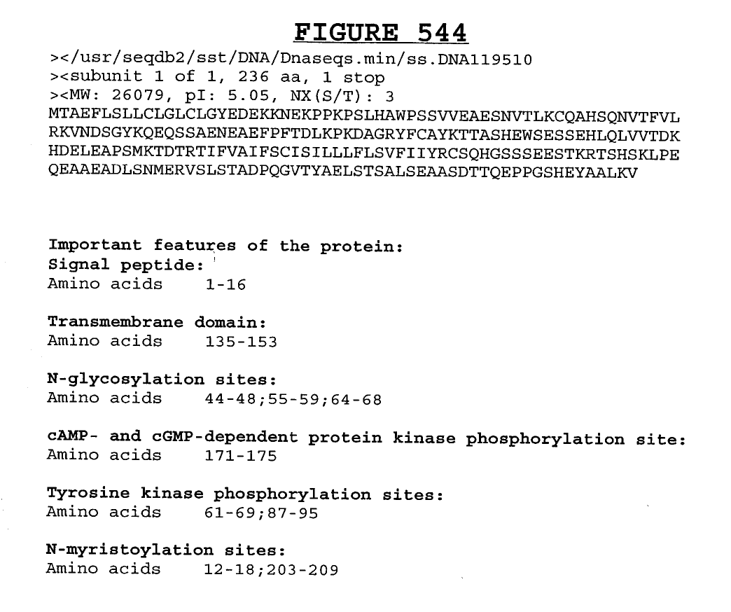

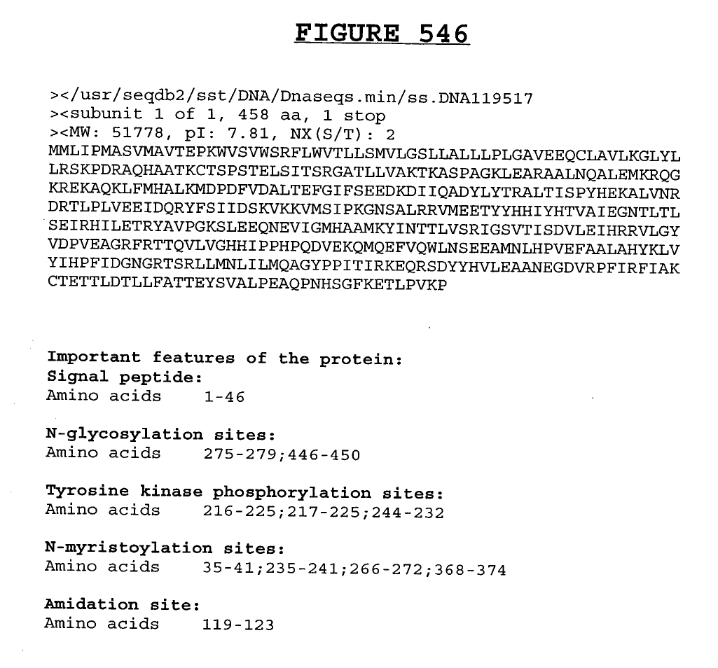

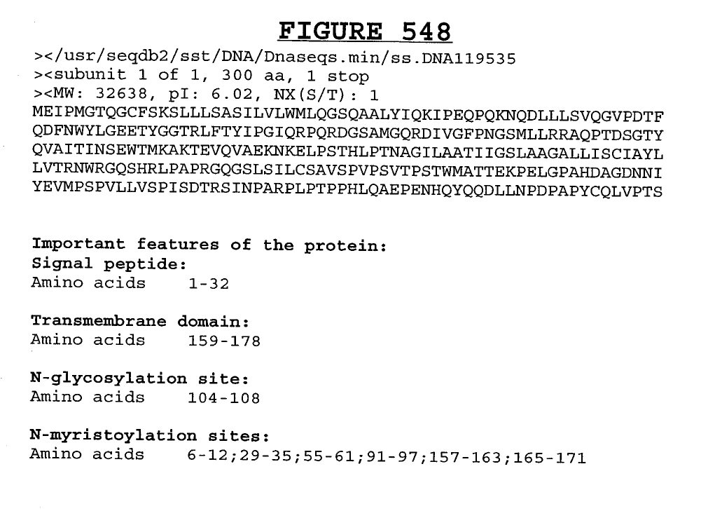

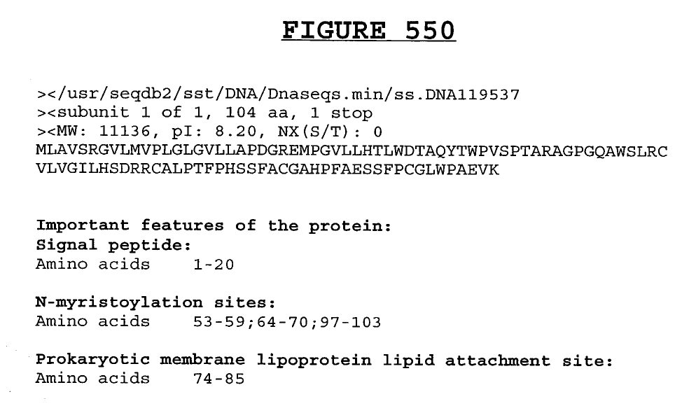

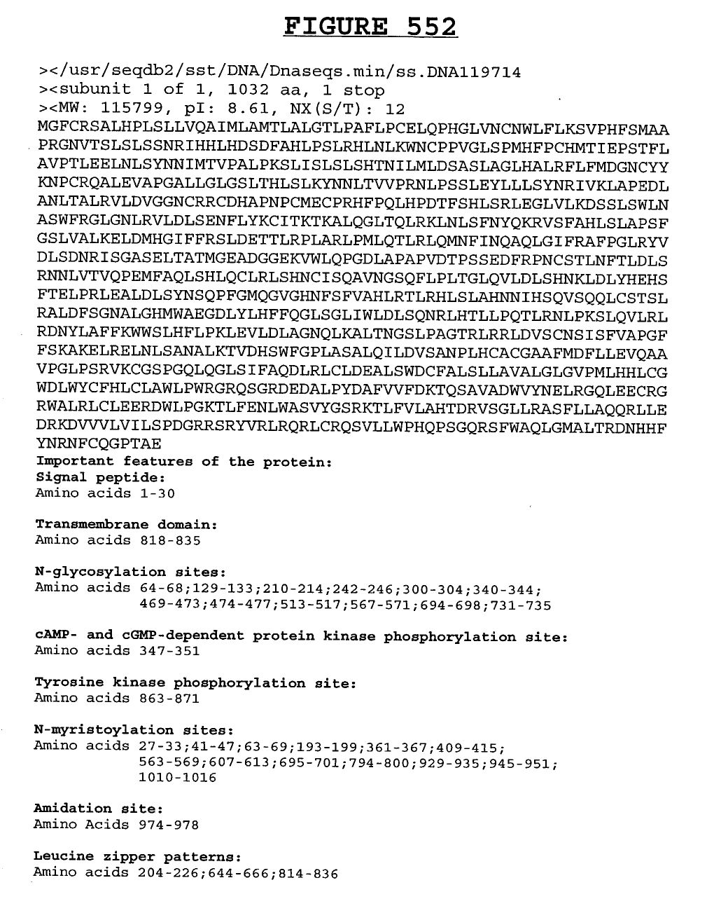

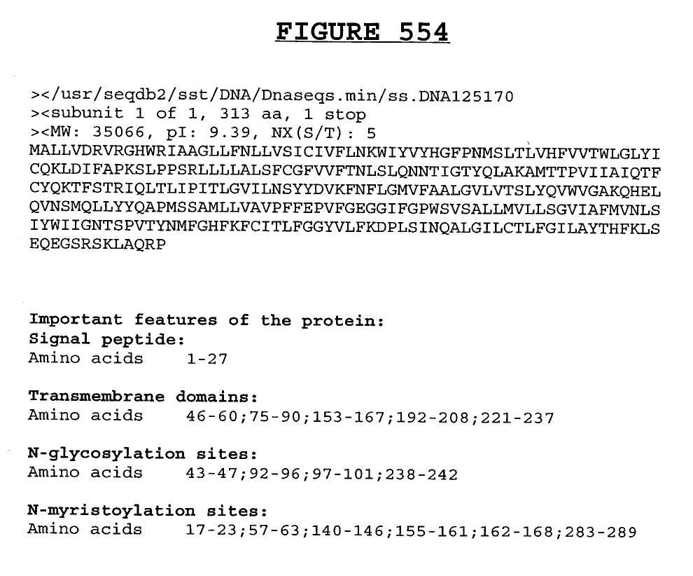

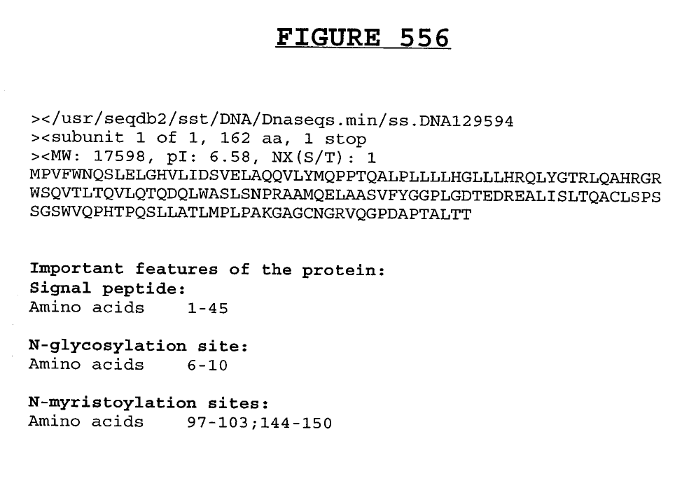

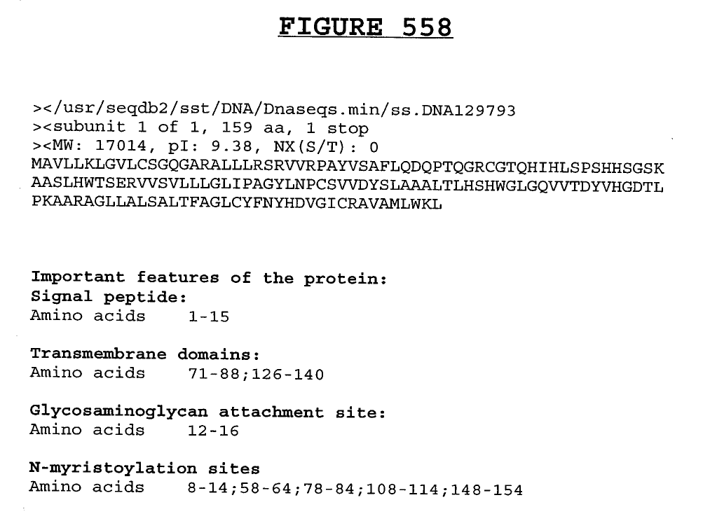

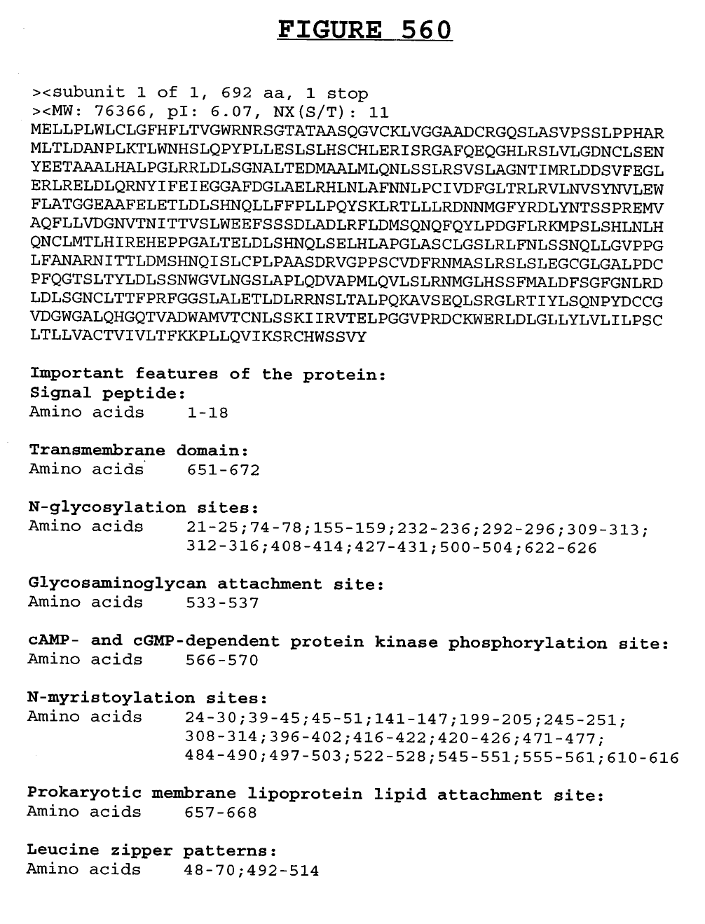

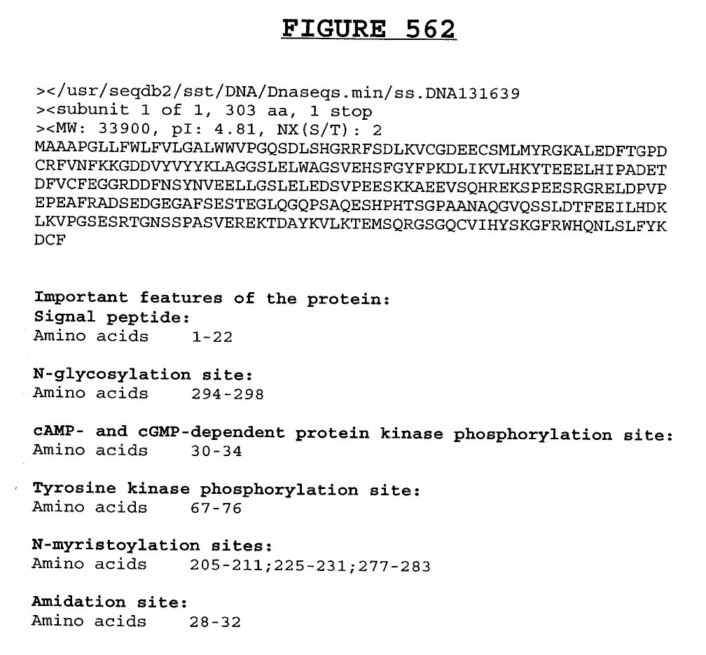

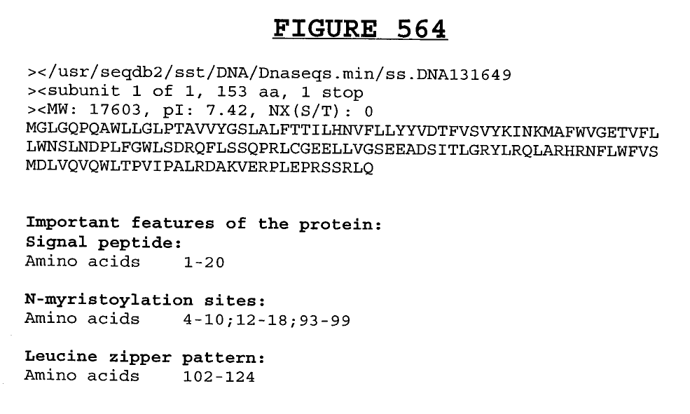

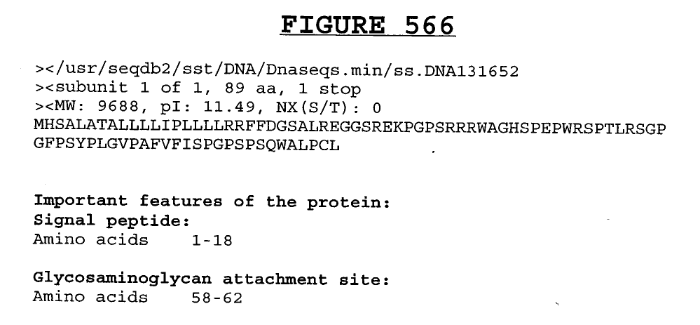

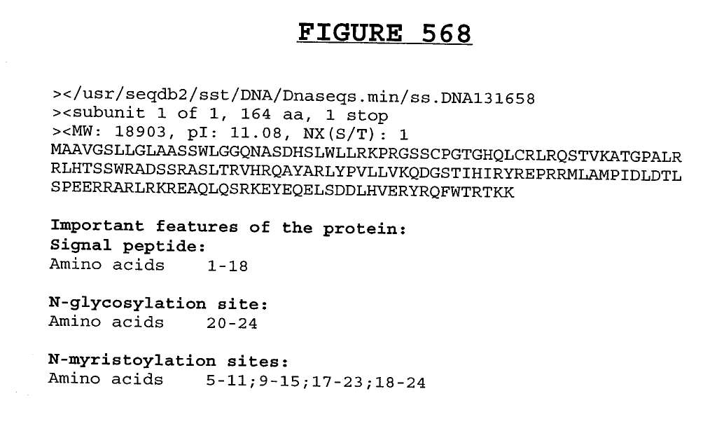

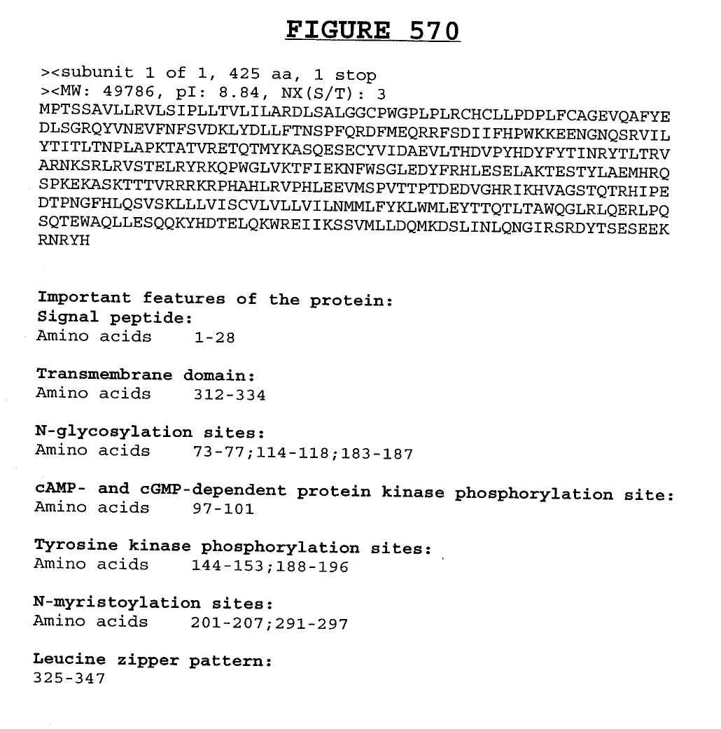

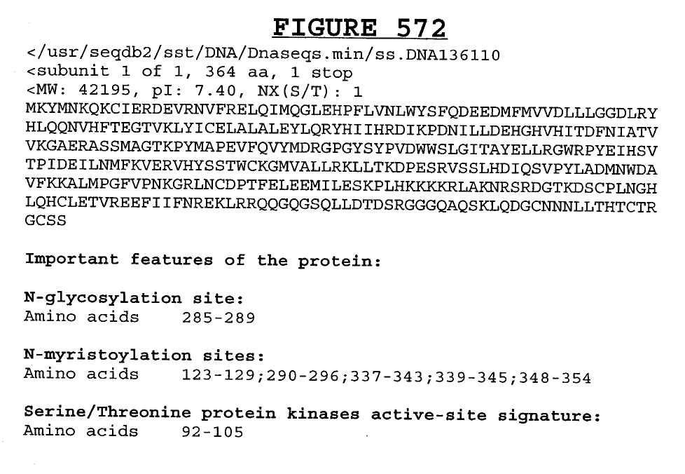

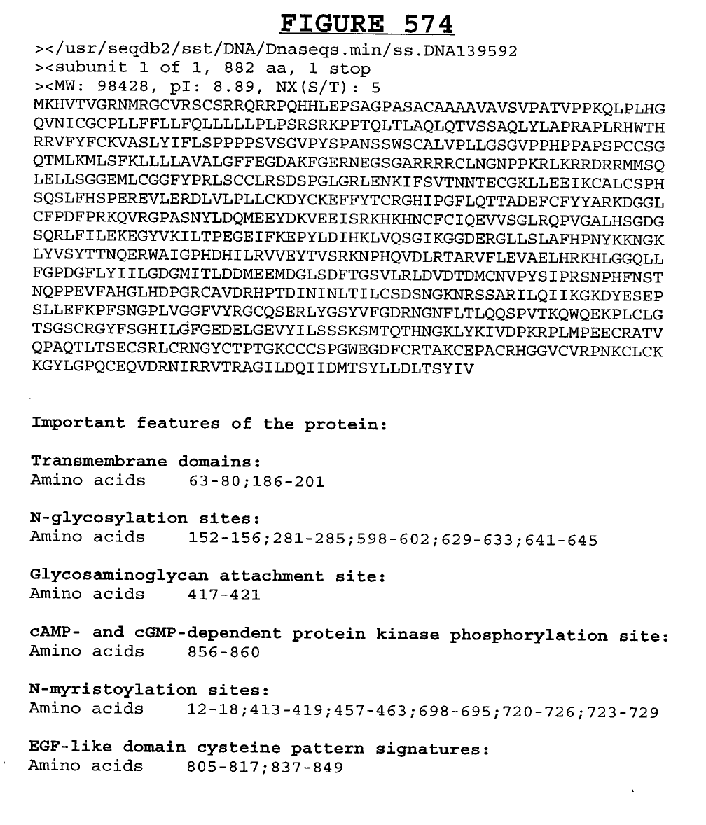

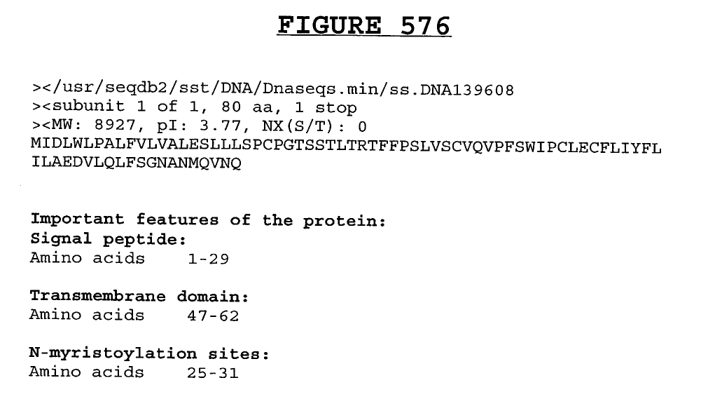

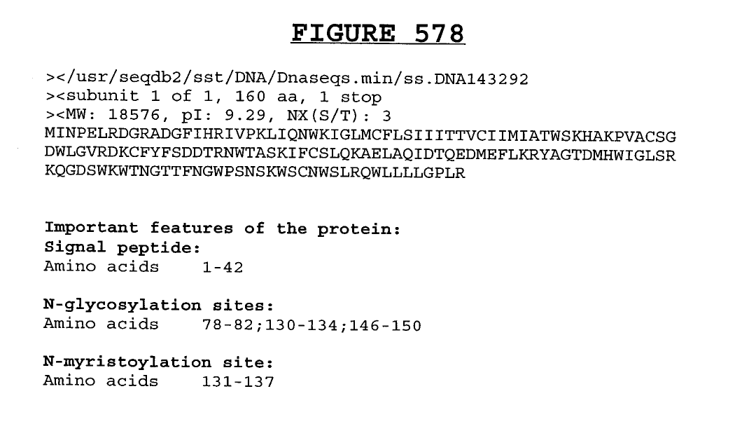

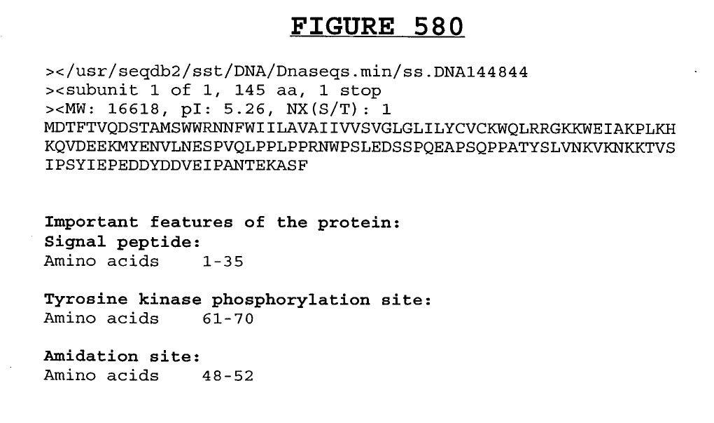

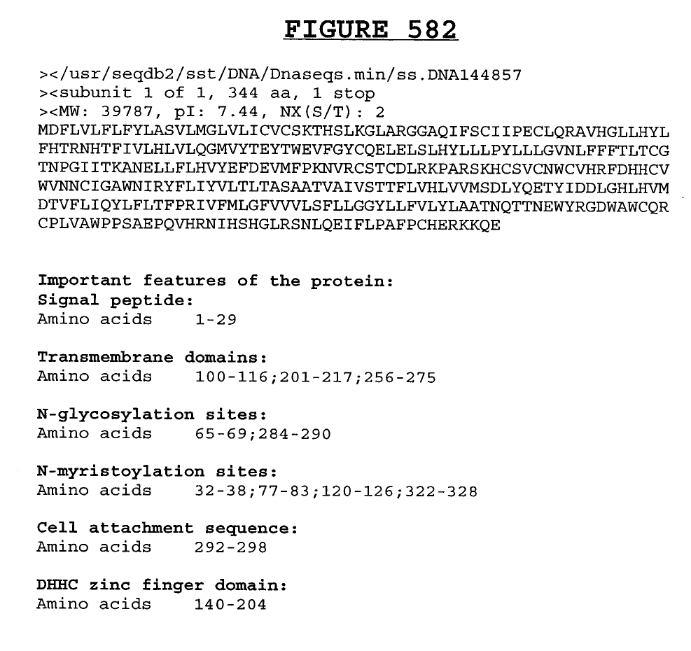

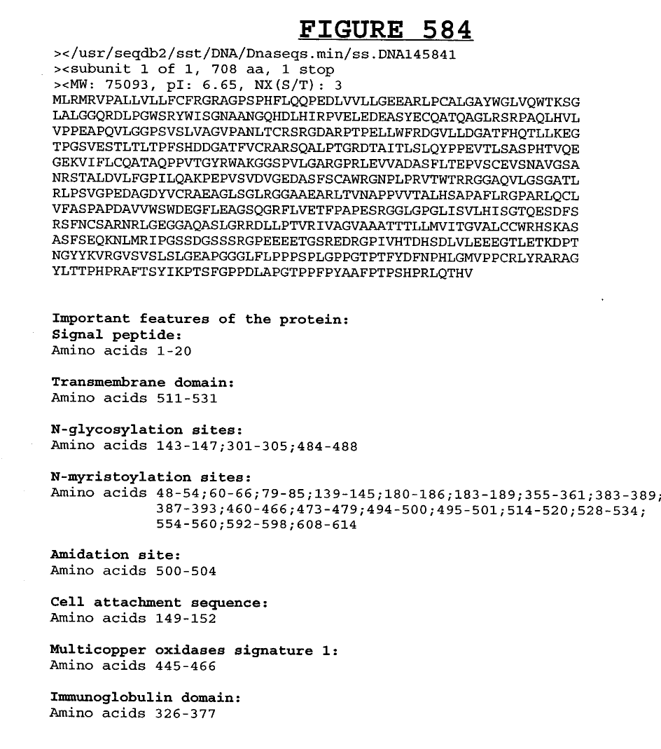

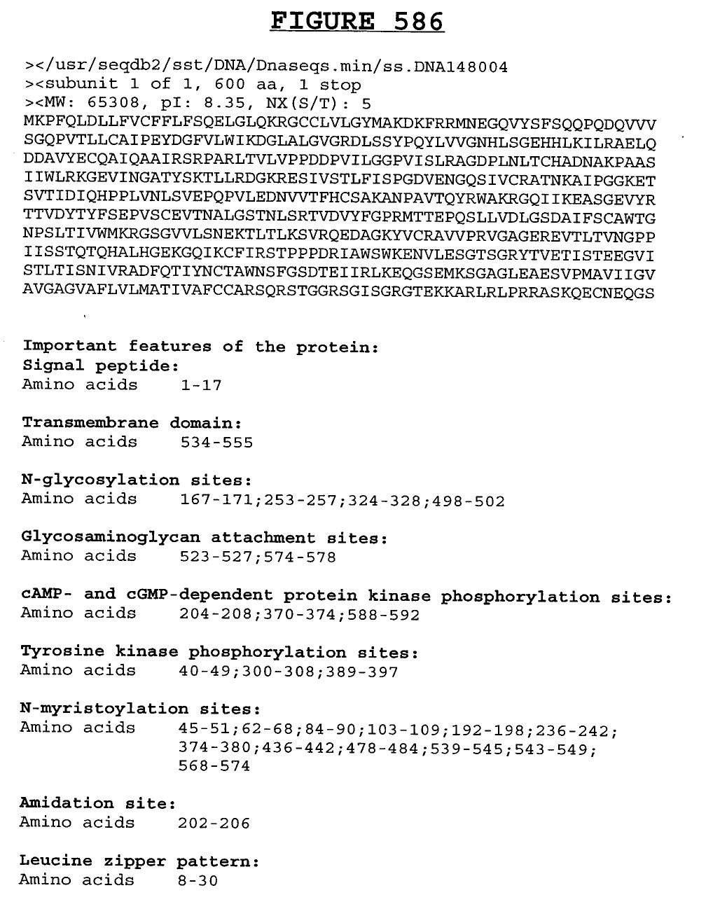

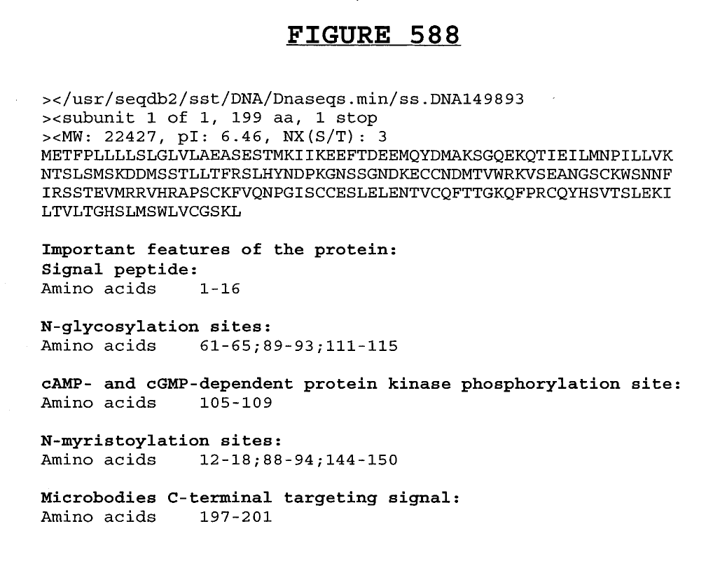

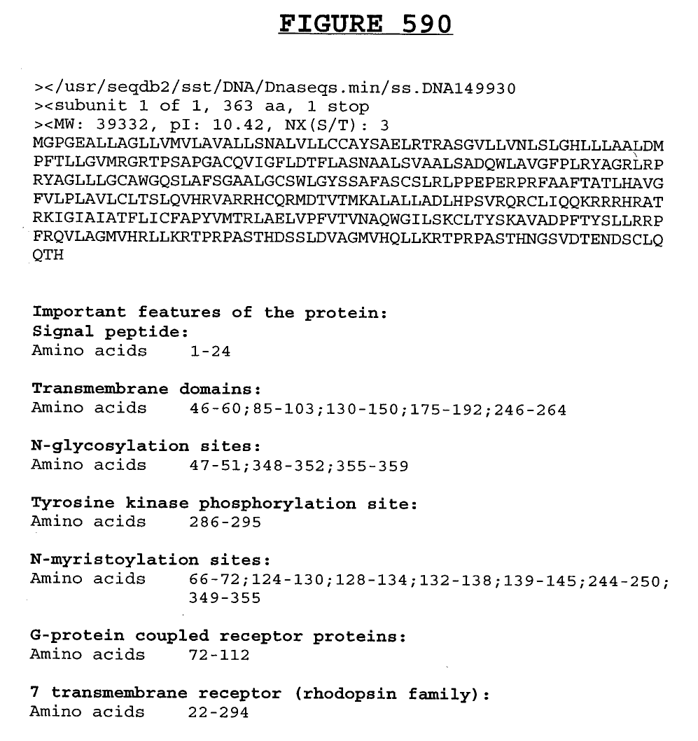

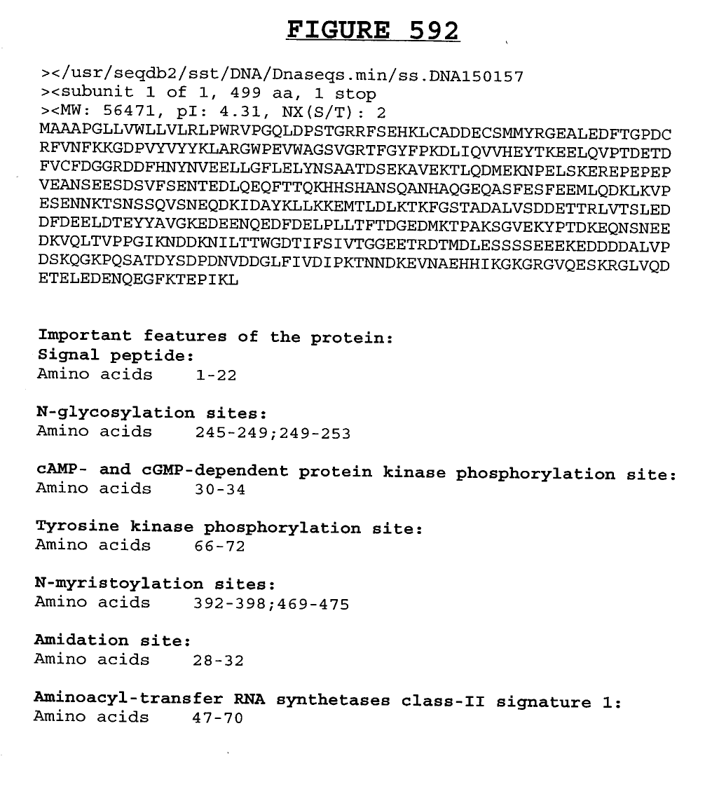

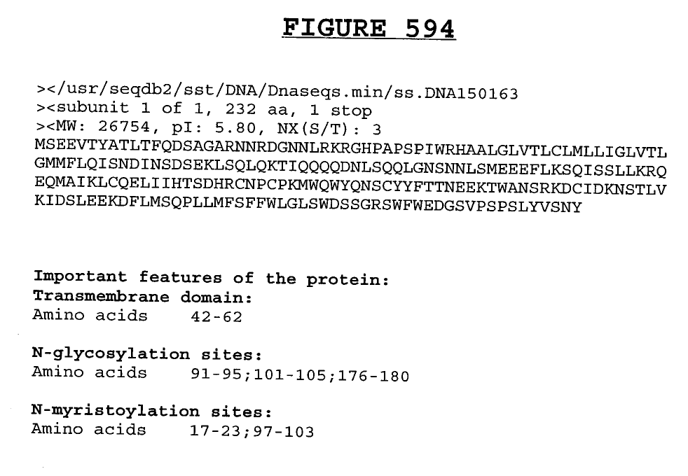

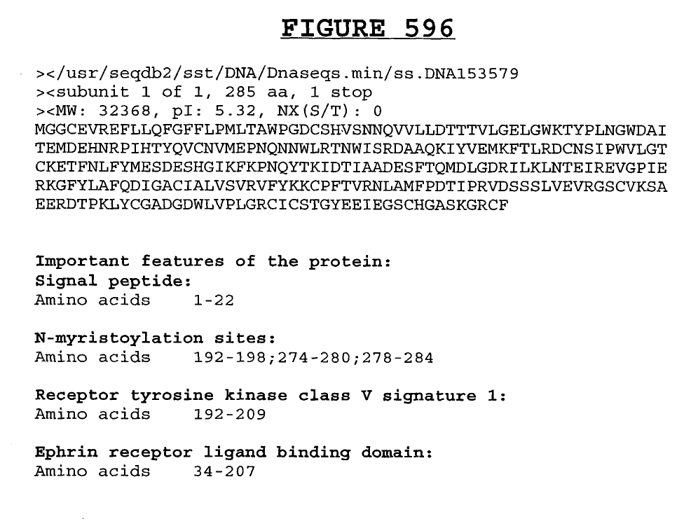

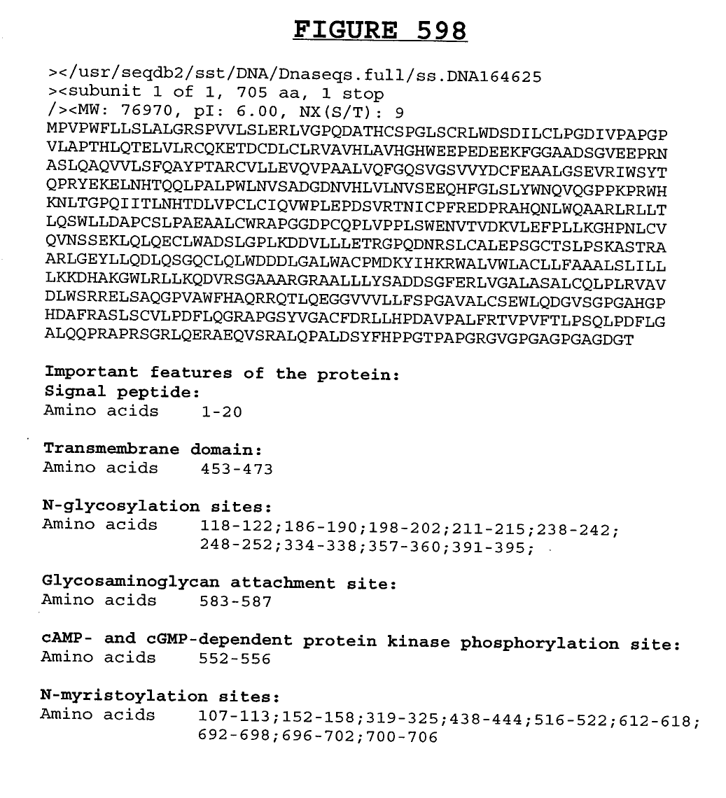

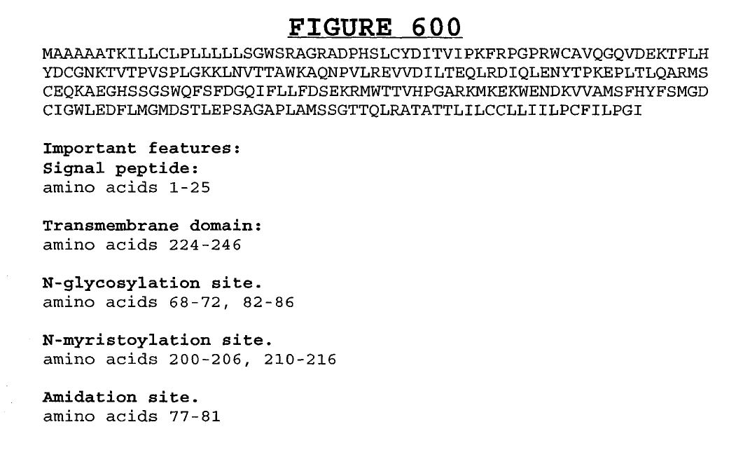

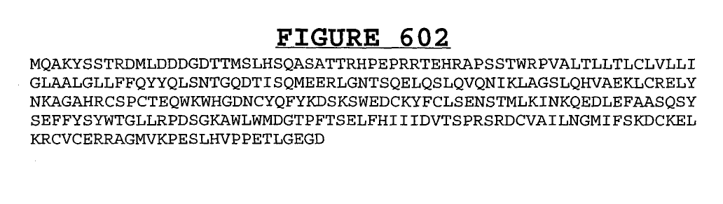

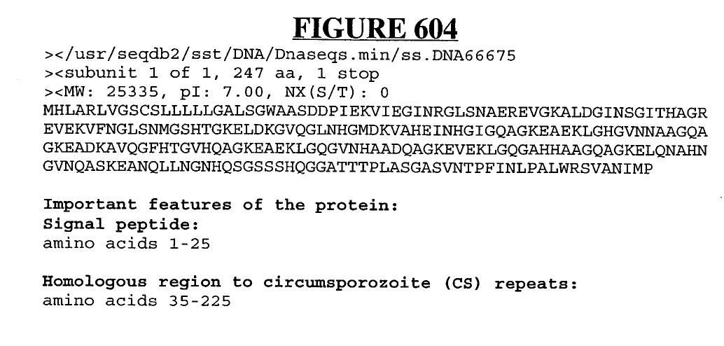

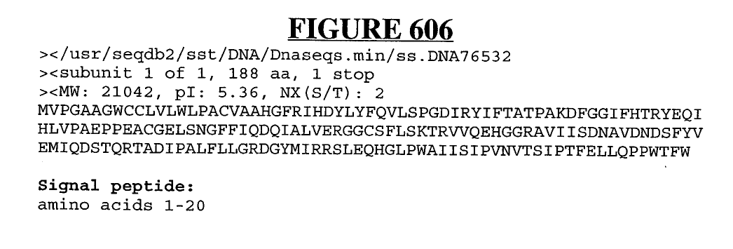

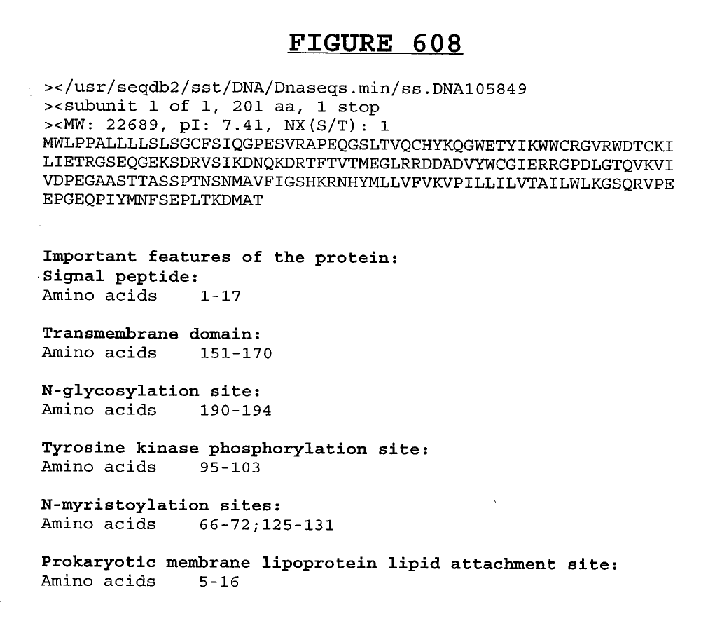

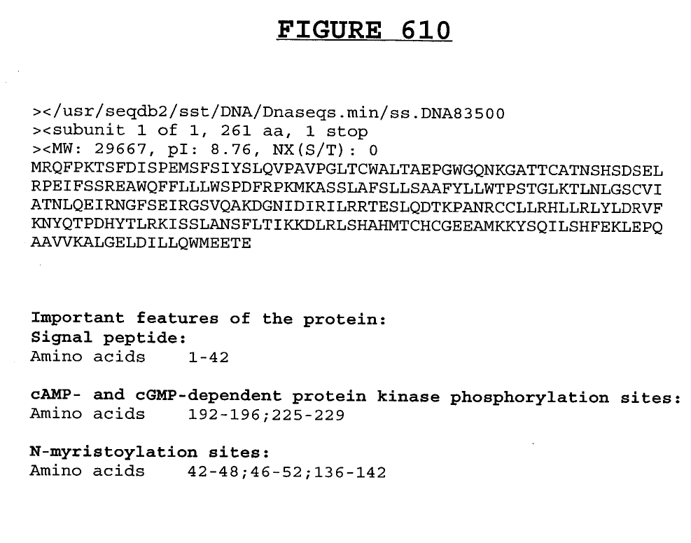

1. Isolated nucleic acid having at least 80% nucleic acid sequence identity to a nucleotide sequence that encodes an amino acid sequence selected from the group consisting of the amino acid sequence shown in FIG. 2 (SEQ ID NO: 2), FIG. 4 (SEQ ID NO: 4), FIG. 6 (SEQ ID NO: 6), FIG. 8 (SEQ ID NO: 8), FIG. 10 (SEQ ID NO: 10), FIG. 12 (SEQ ID NO: 12), FIG. 14 (SEQ ID NO: 14), FIG. 16 (SEQ ID NO: 16), FIG. 18 (SEQ ID NO: 18), FIG. 20 (SEQ ID NO: 20), FIG. 22 (SEQ ID NO: 22), FIG. 24 (SEQ ID NO: 24), FIG. 26 (SEQ ID NO: 26), FIG. 28 (SEQ ID NO: 28), FIG. 30 (SEQ ID NO: 30), FIG. 32 (SEQ ID NO: 32), FIG. 34 (SEQ ID NO: 34), FIG. 36 (SEQ ID NO: 36), FIG. 38 (SEQ ID NO: 38), FIG. 40 (SEQ ID NO: 40), FIG. 42 (SEQ ID NO: 42), FIG. 44 (SEQ ID NO: 44), FIG. 46 (SEQ ID NO: 46), FIG. 48 (SEQ ID NO: 48), FIG. 50 (SEQ ID NO: 50), FIG. 52 (SEQ ID NO: 52), FIG. 54 (SEQ ID NO: 54), FIG. 56 (SEQ ID NO: 56), FIG. 58 (SEQ ID NO: 58), FIG. 60 (SEQ ID NO: 60), FIG. 62 (SEQ ID NO: 62), FIG. 64 (SEQ ID NO: 64), FIG. 66 (SEQ ID NO: 66), FIG. 68 (SEQ ID NO: 68), FIG. 70 (SEQ ID NO: 70), FIG. 72 (SEQ ID NO: 72), FIG. 74 (SEQ ID NO: 74), FIG. 76 (SEQ ID NO: 76), FIG. 78 (SEQ ID NO: 78), FIG. 80 (SEQ ID NO: 80), FIG. 82 (SEQ ID NO: 82), FIG. 84 (SEQ ID NO: 84), FIG. 86 (SEQ ID NO: 86), FIG. 88 (SEQ ID NO: 88), FIG. 90 (SEQ ID NO: 90), FIG. 92 (SEQ ID NO: 92), FIG. 94 (SEQ ID NO: 94), FIG. 96 (SEQ ID NO: 96), FIG. 98 (SEQ ID NO: 98), FIG. 100 (SEQ ID NO: 100), FIG. 102 (SEQ ID NO: 102), FIG. 104 (SEQ ID NO: 104), FIG. 106 (SEQ ID NO: 106), FIG. 108 (SEQ ID NO: 108), FIG. 110 (SEQ ID NO: 110), FIG. 112 (SEQ ID NO: 112), FIG. 114 (SEQ ID NO: 114), FIG. 116 (SEQ ID NO: 116), FIG. 118 (SEQ ID NO: 118), FIG. 120 (SEQ ID NO: 120), FIG. 122 (SEQ ID NO: 122), FIG. 124 (SEQ ID NO: 124), FIG. 126 (SEQ ID NO: 126), FIG. 128 (SEQ ID NO: 128), FIG. 130 (SEQ ID NO: 130), FIG. 132 (SEQ ID NO: 132), FIG. 134 (SEQ ID NO: 134), FIG. 136 (SEQ ID NO: 136), FIG. 138 (SEQ ID NO: 138), FIG. 140 (SEQ ID NO: 140), FIG. 142 (SEQ ID NO: 142), FIG. 144 (SEQ ID NO: 144), FIG. 146 (SEQ ID NO: 146), FIG. 148 (SEQ ID NO: 148), FIG. 150 (SEQ ID NO: 150), FIG. 152 (SEQ ID NO: 152), FIG. 154 (SEQ ID NO: 154), FIG. 156 (SEQ ID NO: 156), FIG. 158 (SEQ ID NO: 158), FIG. 160 (SEQ ID NO: 160), FIG. 162 (SEQ ID NO: 162), FIG. 164 (SEQ ID NO: 164), FIG. 166 (SEQ ID NO: 166), FIG. 168 (SEQ ID NO: 168), FIG. 170 (SEQ ID NO: 170), FIG. 172 (SEQ ID NO: 172), FIG. 174 (SEQ ID NO: 174), FIG. 176 (SEQ ID NO: 176), FIG. 178 (SEQ ID NO: 178), FIG. 180 (SEQ ID NO: 180), FIG. 182 (SEQ ID NO: 182), FIG. 184 (SEQ ID NO: 184), FIG. 186 (SEQ ID NO: 186), FIG. 188 (SEQ ID NO: 188), FIG. 190 (SEQ ID NO: 190), FIG. 192 (SEQ ID NO: 192), FIG. 194 (SEQ ID NO: 194), FIG. 196 (SEQ ID NO: 196), FIG. 198 (SEQ ID NO: 198), FIG. 200 (SEQ ID NO: 200), FIG. 202 (SEQ ID NO: 202), FIG. 204 (SEQ ID NO: 204), FIG. 206 (SEQ ID NO: 206), FIG. 208 (SEQ ID NO: 208), FIG. 210 (SEQ ID NO: 210), FIG. 212 (SEQ ID NO: 212), FIG. 214 (SEQ ID NO: 214), FIG. 216 (SEQ ID NO: 216), FIG. 218 (SEQ ID NO: 218), FIG. 220 (SEQ ID NO: 220), FIG. 222 (SEQ ID NO: 222), FIG. 224 (SEQ ID NO: 224), FIG. 226 (SEQ ID NO: 226), FIG. 228 (SEQ ID NO: 228), FIG. 230 (SEQ ID NO: 230), FIG. 232 (SEQ ID NO: 232), FIG. 234 (SEQ ID NO: 234), FIG. 236 (SEQ ID NO: 236), FIG. 238 (SEQ ID NO: 238), FIG. 240 (SEQ ID NO: 240), FIG. 242 (SEQ ID NO: 242), FIG. 244 (SEQ ID NO: 244), FIG. 246 (SEQ ID NO: 246), FIG. 248 (SEQ ID NO: 248), FIG. 250 (SEQ ID NO: 250), FIG. 252 (SEQ ID NO: 252), FIG. 254 (SEQ ID NO: 254), FIG. 256 (SEQ ID NO: 256), FIG. 258 (SEQ ID NO: 258), FIG. 260 (SEQ ID NO: 260), FIG. 262 (SEQ ID NO: 262), FIG. 264 (SEQ ID NO: 264), FIG. 266 (SEQ ID NO: 266), FIG. 268 (SEQ ID NO: 268), FIG. 270 (SEQ ID NO: 270), FIG. 272 (SEQ ID NO: 272), FIG. 274 (SEQ ID NO: 274), FIG. 276 (SEQ ID NO: 276), FIG. 278 (SEQ ID NO: 278), FIG. 280 (SEQ ID NO: 280), FIG. 282 (SEQ ID NO: 282), FIG. 284 (SEQ ID NO: 284), FIG. 286 (SEQ ID NO: 286), FIG. 288 (SEQ ID NO: 288), FIG. 290 (SEQ ID NO: 290), FIG. 292 (SEQ ID NO: 292), FIG. 294 (SEQ ID NO: 294), FIG. 296 (SEQ ID NO: 296), FIG. 298 (SEQ ID NO: 298), FIG. 300 (SEQ ID NO: 300), FIG. 302 (SEQ ID NO: 302), FIG. 304 (SEQ ID NO: 304), FIG. 306 (SEQ ID NO: 306), FIG. 308 (SEQ ID NO: 308), FIG. 310 (SEQ ID NO: 310), FIG. 312 (SEQ ID NO: 312), FIG. 314 (SEQ ID NO: 314), FIG. 316 (SEQ ID NO: 316), FIG. 318 (SEQ ID NO: 318), FIG. 320 (SEQ ID NO: 320), FIG. 322 (SEQ ID NO: 322), FIG. 324 (SEQ ID NO: 324), FIG. 326 (SEQ ID NO: 326), FIG. 328 (SEQ ID NO: 328), FIG. 330 (SEQ ID NO: 330), FIG. 332 (SEQ ID NO: 332), FIG. 334 (SEQ ID NO: 334), FIG. 336 (SEQ ID NO: 336), FIG. 338 (SEQ ID NO: 338), FIG. 340 (SEQ ID NO: 340), FIG. 342 (SEQ ID NO: 342), FIG. 344 (SEQ ID NO: 344), FIG. 346 (SEQ ID NO: 346), FIG. 348 (SEQ ID NO: 348), FIG. 350 (SEQ ID NO: 350), FIG. 352 (SEQ ID NO: 352), FIG. 354 (SEQ ID NO: 354), FIG. 356 (SEQ ID NO: 356), FIG. 358 (SEQ ID NO: 358), FIG. 360 (SEQ ID NO: 360), FIG. 362 (SEQ ID NO: 362), FIG. 364 (SEQ ID NO: 364), FIG. 366 (SEQ ID NO: 366), FIG. 368 (SEQ ID NO: 368), FIG. 370 (SEQ ID NO: 370), FIG. 372 (SEQ ID NO: 372), FIG. 374 (SEQ ID NO: 374), FIG. 376 (SEQ ID NO: 376), FIG. 378 (SEQ ID NO: 378), FIG. 380 (SEQ ID NO: 380), FIG. 382 (SEQ ID NO: 382), FIG. 384 (SEQ ID NO: 384), FIG. 386 (SEQ ID NO: 386), FIG. 388 (SEQ ID NO: 388), FIG. 390 (SEQ ID NO: 390), FIG. 392 (SEQ ID NO: 392), FIG. 394 (SEQ ID NO: 394), FIG. 396 (SEQ ID NO: 396), FIG. 398 (SEQ ID NO: 398), FIG. 400 (SEQ ID NO: 400), FIG. 402 (SEQ ID NO: 402), FIG. 404 (SEQ ID NO: 404), FIG. 406 (SEQ ID NO: 406), FIG. 408 (SEQ ID NO: 408), FIG. 410 (SEQ ID NO: 410), FIG. 412 (SEQ ID NO: 412), FIG. 414 (SEQ ID NO: 414), FIG. 416 (SEQ ID NO: 416), FIG. 418 (SEQ ID NO: 418), FIG. 420 (SEQ ID NO: 420), FIG. 422 (SEQ ID NO: 422), FIG. 424 (SEQ ID NO: 424), FIG. 426 (SEQ ID NO: 426), FIG. 428 (SEQ ID NO: 428), FIG. 430 (SEQ ID NO: 430), FIG. 432 (SEQ ID NO: 432), FIG. 434 (SEQ ID NO: 434), FIG. 436 (SEQ ID NO: 436), FIG. 438 (SEQ ID NO: 438), FIG. 440 (SEQ ID NO: 440), FIG. 442 (SEQ ID NO: 442), FIG. 444 (SEQ ID NO: 444), FIG. 446 (SEQ ID NO: 446), FIG. 448 (SEQ ID NO: 448), FIG. 450 (SEQ ID NO: 450), FIG. 452 (SEQ ID NO: 452), FIG. 454 (SEQ ID NO: 454), FIG. 456 (SEQ ID NO: 456), FIG. 458 (SEQ ID NO: 458), FIG. 460 (SEQ ID NO: 460), FIG. 462 (SEQ ID NO: 462), FIG. 464 (SEQ ID NO: 464), FIG. 466 (SEQ ID NO: 466), FIG. 468 (SEQ ID NO: 468), FIG. 470 (SEQ ID NO: 470), FIG. 472 (SEQ ID NO: 472), FIG. 474 (SEQ ID NO: 474), FIG. 476 (SEQ ID NO: 476), FIG. 478 (SEQ ID NO: 478), FIG. 480 (SEQ ID NO: 480), FIG. 482 (SEQ ID NO: 482), FIG. 484 (SEQ ID NO: 484), FIG. 486 (SEQ ID NO: 486), FIG. 488 (SEQ ID NO: 488), FIG. 490 (SEQ ID NO: 490), FIG. 492 (SEQ ID NO: 492), FIG. 494 (SEQ ID NO: 494), FIG. 496 (SEQ ID NO: 496), FIG. 498 (SEQ ID NO: 498), FIG. 500 (SEQ ID NO: 500), FIG. 502 (SEQ ID NO: 502), FIG. 504 (SEQ ID NO: 504), FIG. 506 (SEQ ID NO: 506), FIG. 508 (SEQ ID NO: 508), FIG. 510 (SEQ ID NO: 510), FIG. 512 (SEQ ID NO: 512), FIG. 514 (SEQ ID NO: 514), FIG. 516 (SEQ ID NO: 516), FIG. 518 (SEQ ID NO: 518), FIG. 520 (SEQ ID NO: 520), FIG. 522 (SEQ ID NO: 522), FIG. 524 (SEQ ID NO: 524), FIG. 526 (SEQ ID NO: 526), FIG. 528 (SEQ ID NO: 528), FIG. 530 (SEQ ID NO: 530), FIG. 532 (SEQ ID NO: 532), FIG. 534 (SEQ ID NO: 534), FIG. 536 (SEQ ID NO: 536), FIG. 538 (SEQ ID NO: 538), FIG. 540 (SEQ ID NO: 540), FIG. 542 (SEQ ID NO: 542), FIG. 544 (SEQ ID NO: 544), FIG. 546 (SEQ ID NO: 546), FIG. 548 (SEQ ID NO: 548), FIG. 550 (SEQ ID NO: 550), FIG. 552 (SEQ ID NO: 552), FIG. 554 (SEQ ID NO: 554), FIG. 556 (SEQ ID NO: 556), FIG. 558 (SEQ ID NO: 558), FIG. 560 (SEQ ID NO: 560), FIG. 562 (SEQ ID NO: 562), FIG. 564 (SEQ ID NO: 564), FIG. 566 (SEQ ID NO: 566), FIG. 568 (SEQ ID NO: 568), FIG. 570 (SEQ ID NO: 570), FIG. 572 (SEQ ID NO: 572), FIG. 574 (SEQ ID NO: 574), FIG. 576 (SEQ ID NO: 576), FIG. 578 (SEQ ID NO: 578), FIG. 580 (SEQ ID NO: 580), FIG. 582 (SEQ ID NO: 582), FIG. 584 (SEQ ID NO: 584), FIG. 586 (SEQ ID NO: 586), FIG. 588 (SEQ ID NO: 588), FIG. 590 (SEQ ID NO: 590), FIG. 592 (SEQ ID NO: 592), FIG. 594 (SEQ ID NO: 594), FIG. 596 (SEQ ID NO: 596), FIG. 598 (SEQ ID NO: 598), FIG. 600 (SEQ ID NO: 600), FIG. 602 (SEQ ID NO: 602), FIG. 604 (SEQ ID NO: 604), FIG. 606 (SEQ ID NO: 606), FIG. 608 (SEQ ID NO: 608), and FIG. 610 (SEQ ID NO: 610).

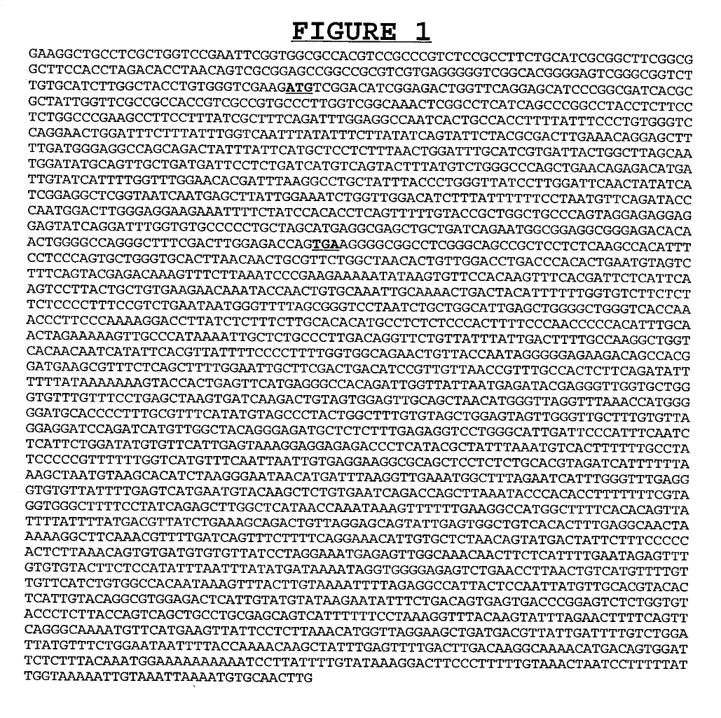

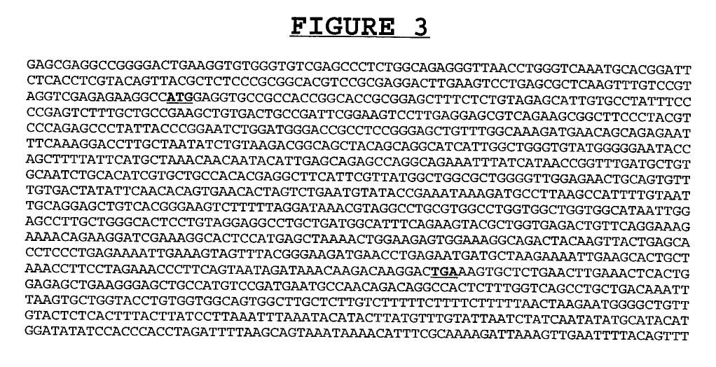

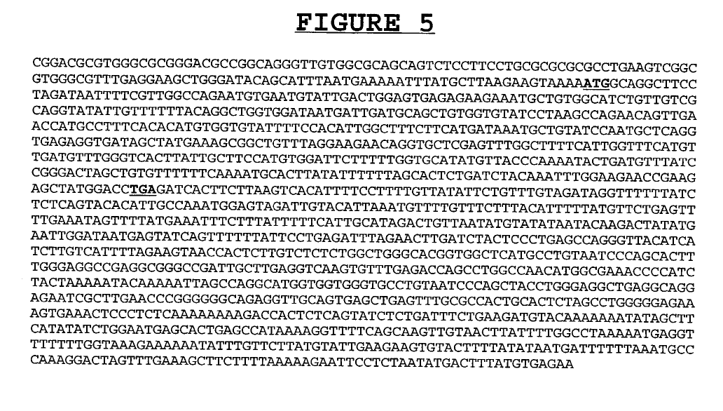

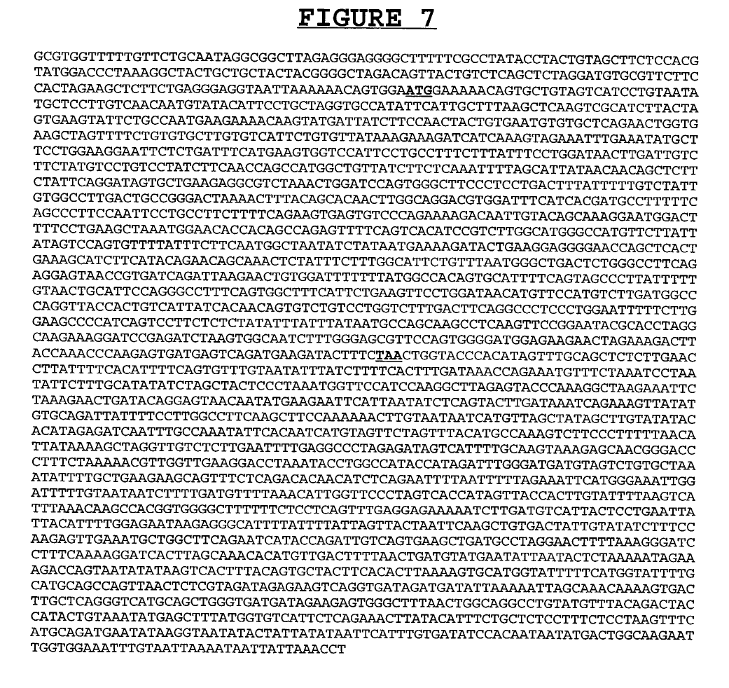

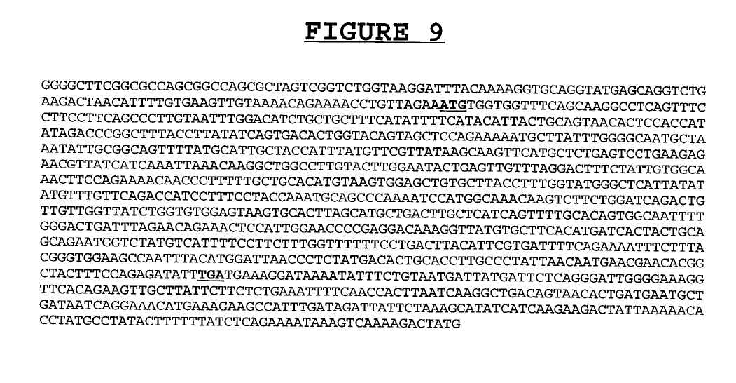

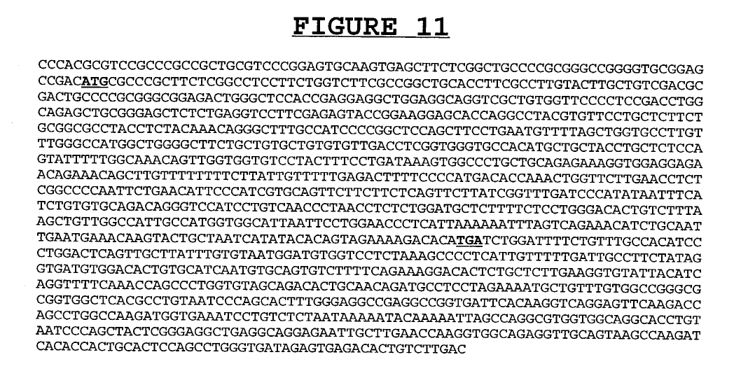

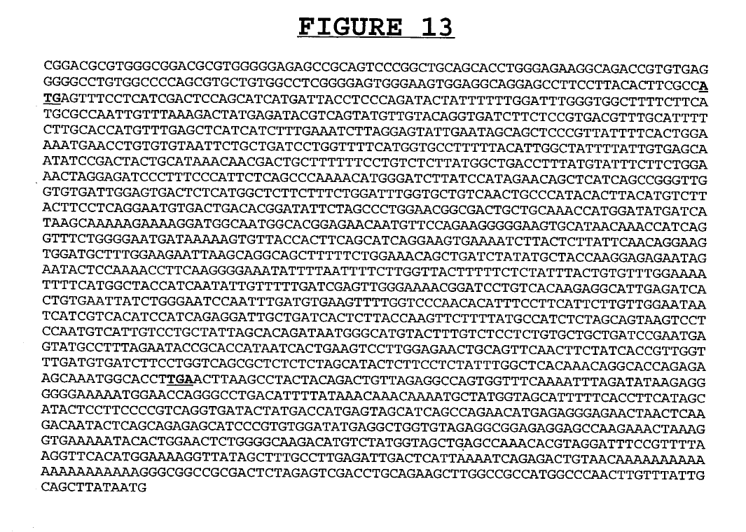

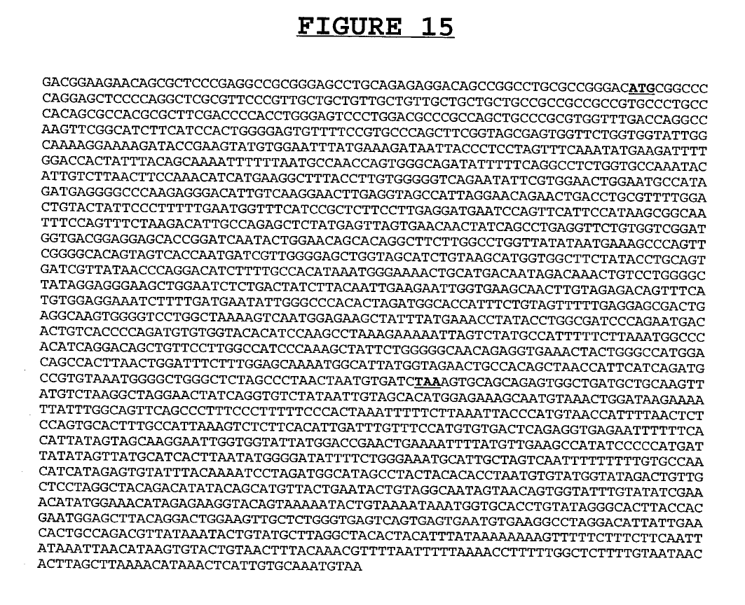

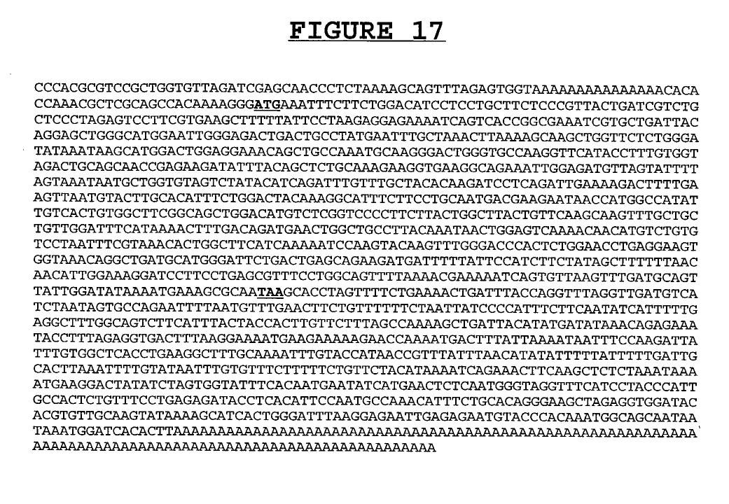

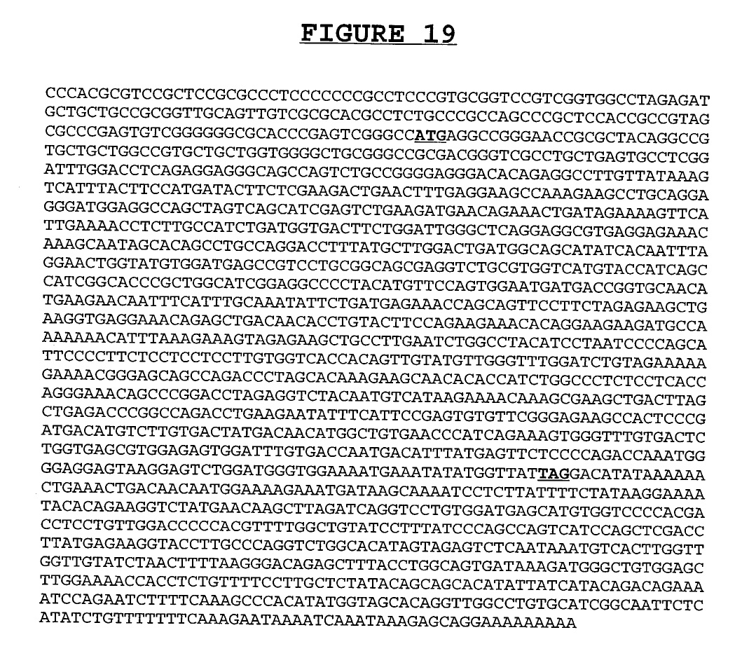

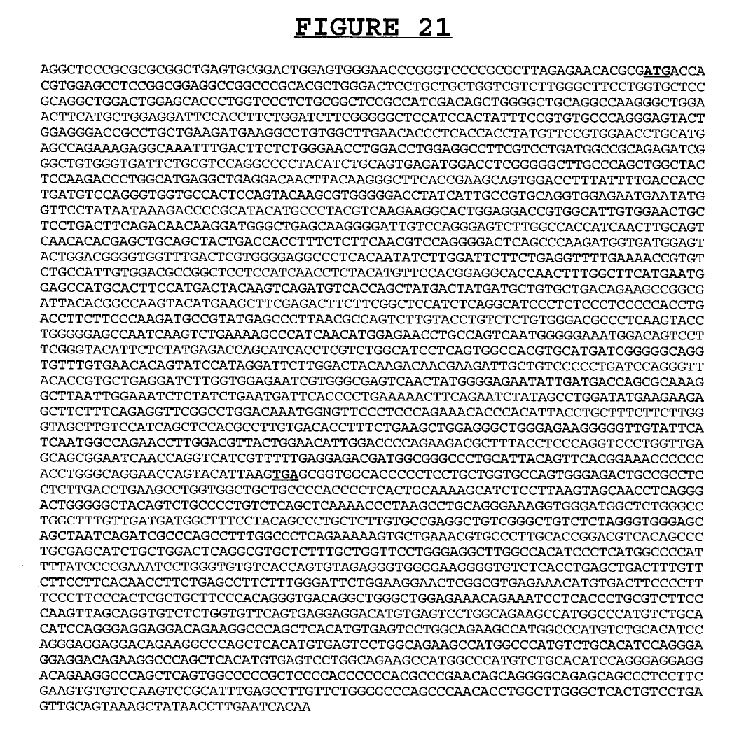

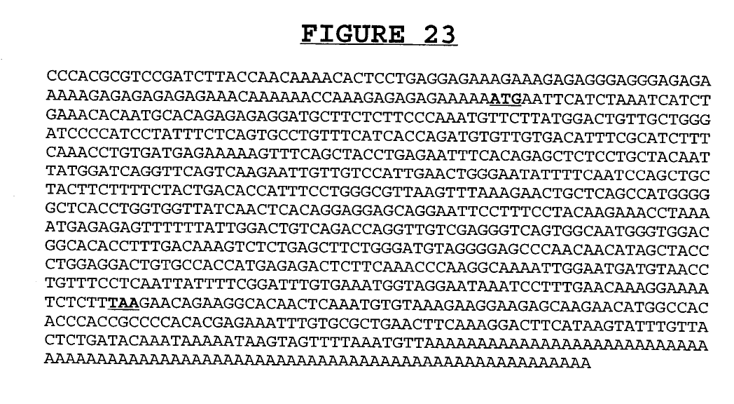

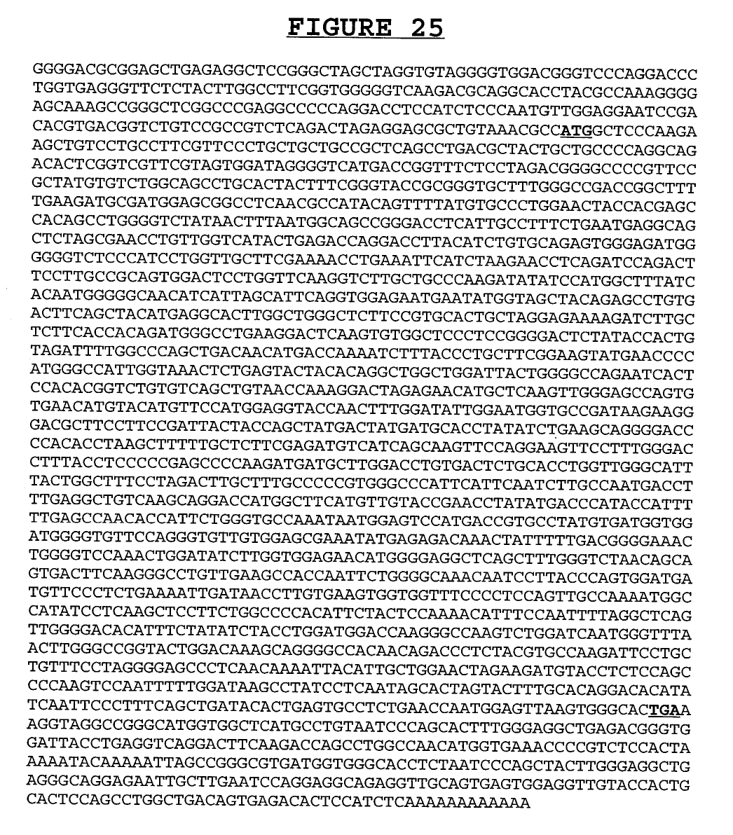

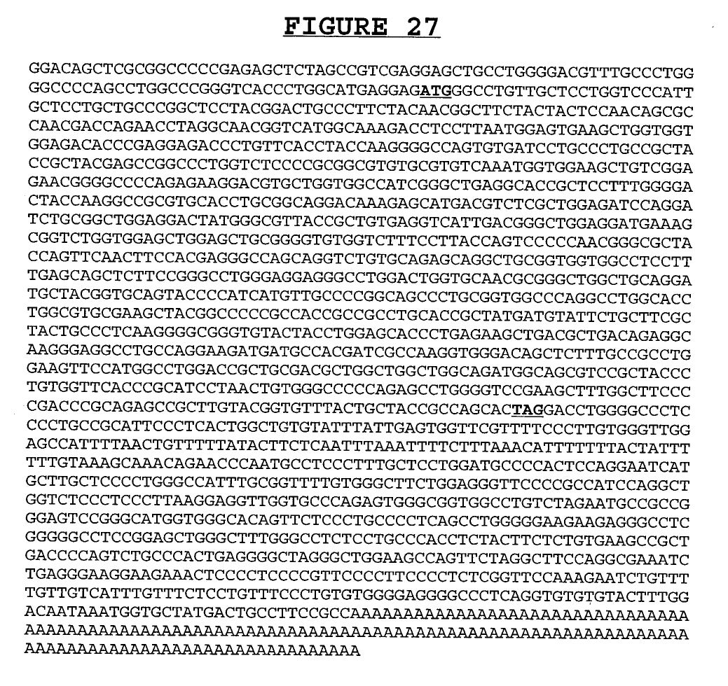

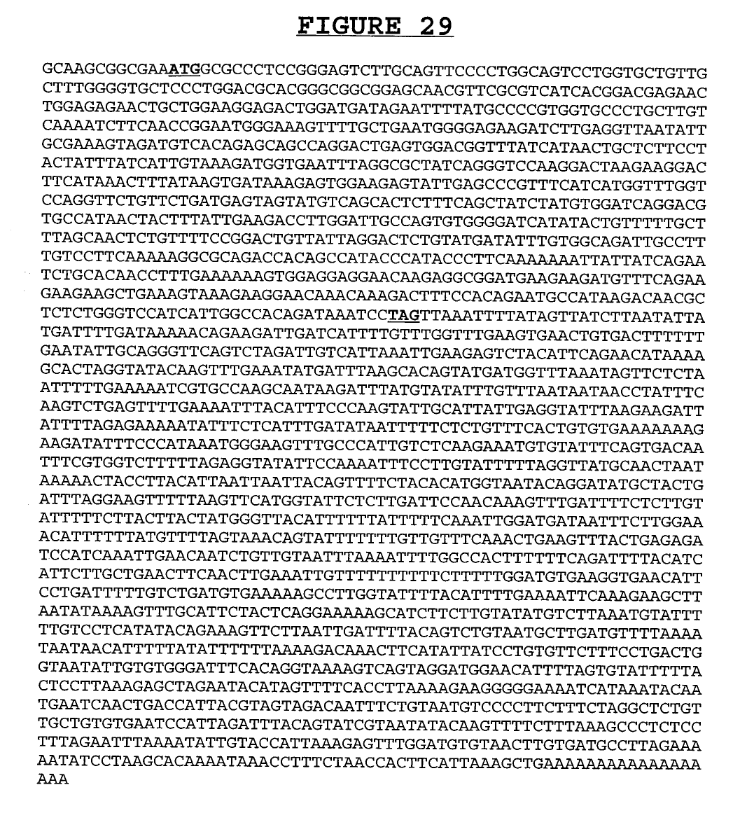

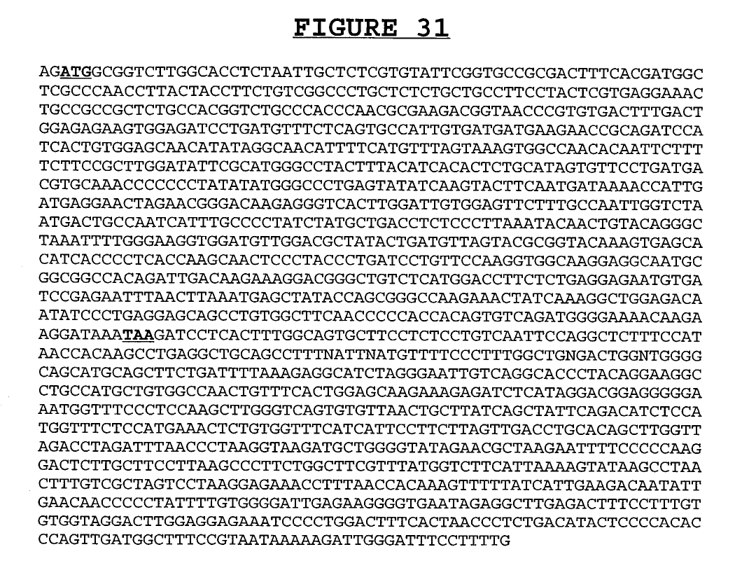

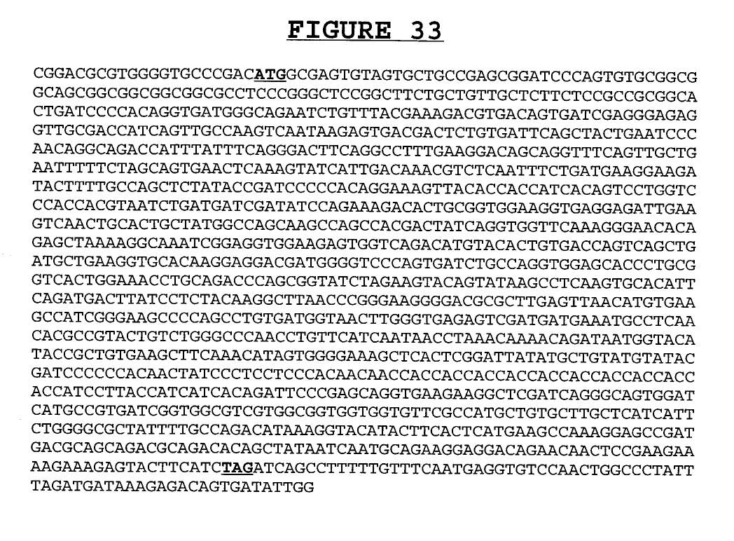

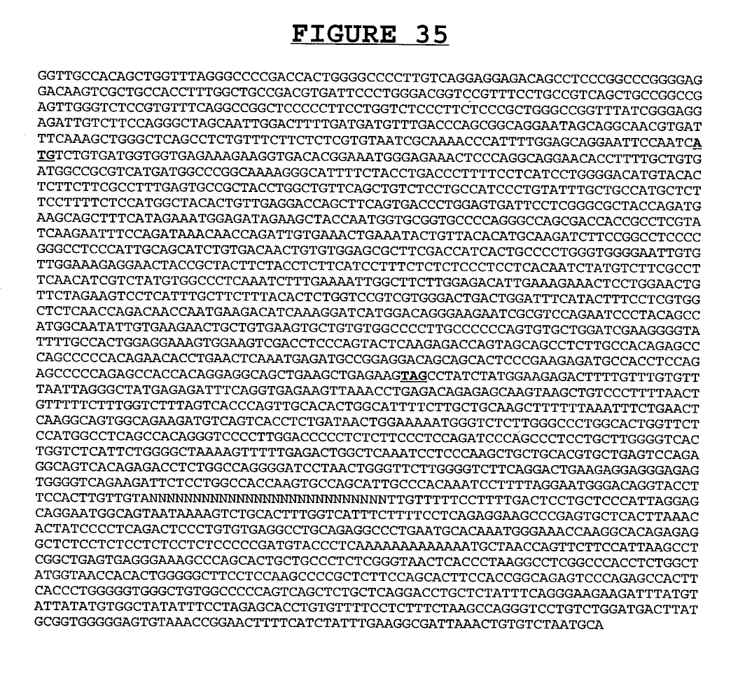

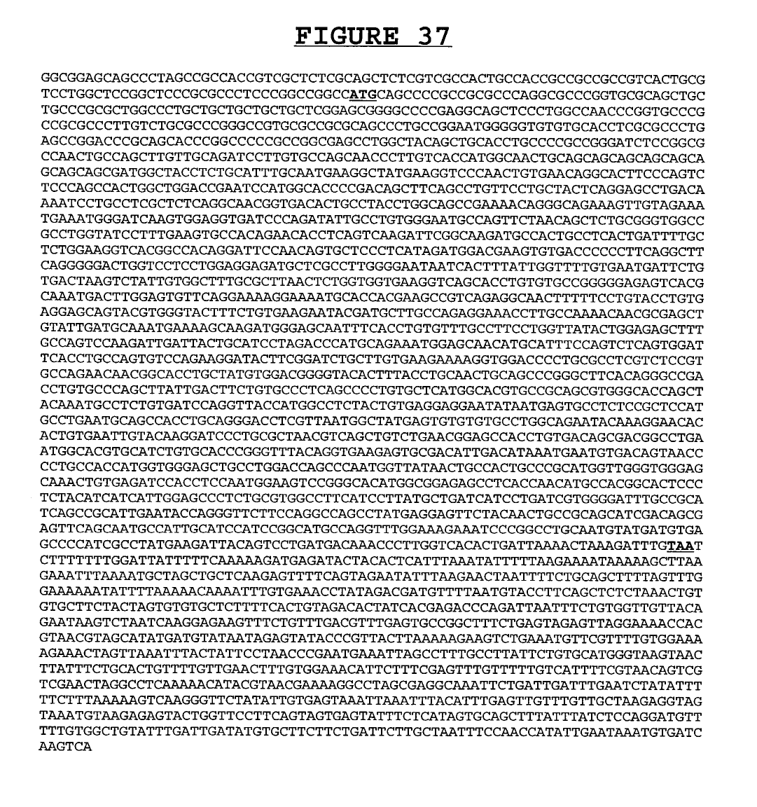

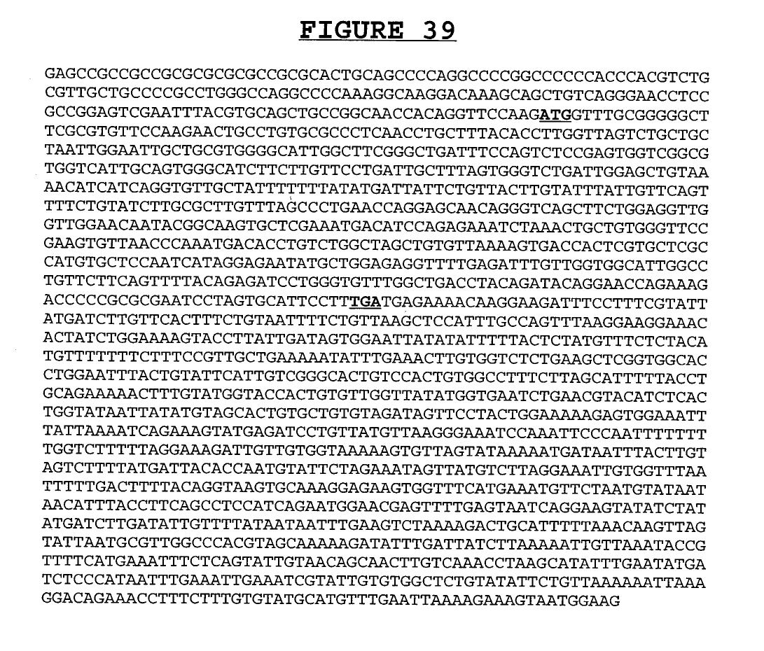

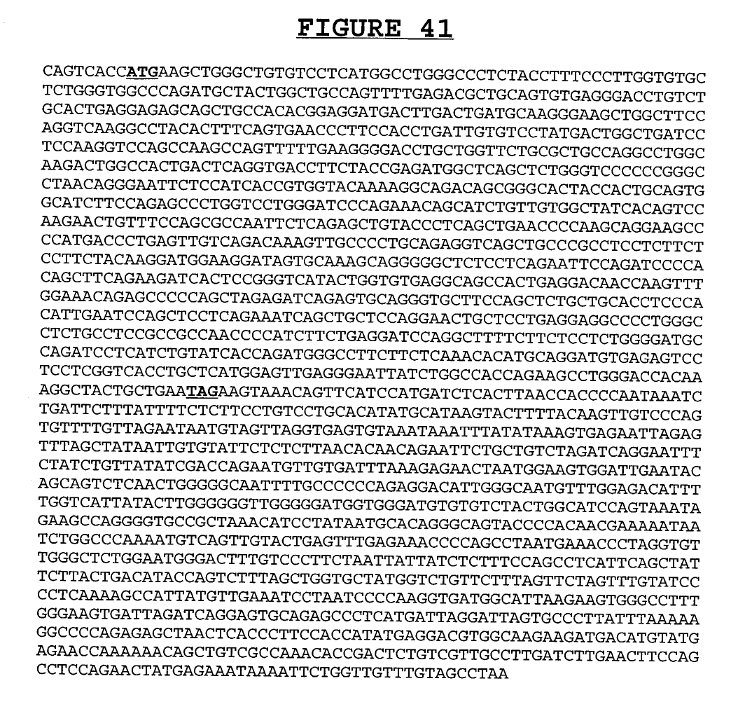

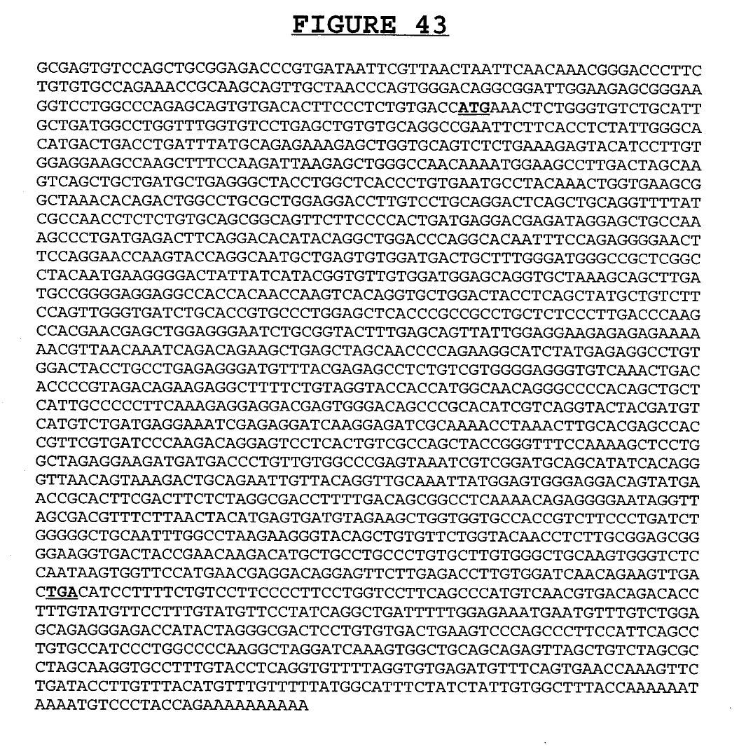

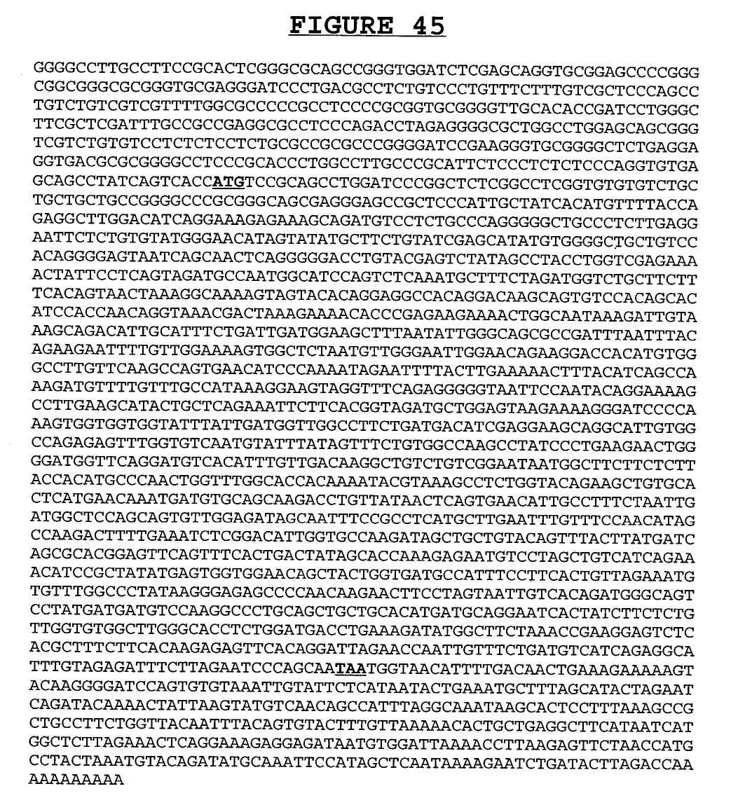

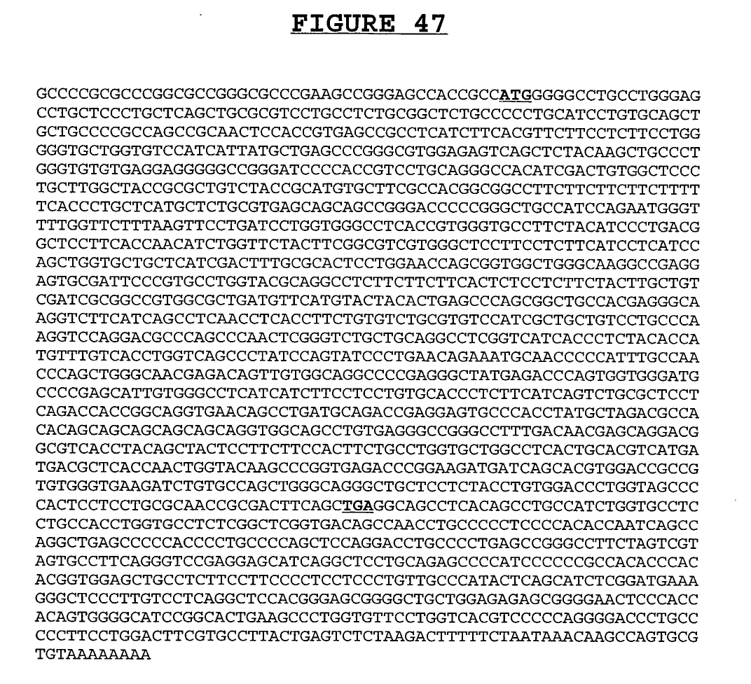

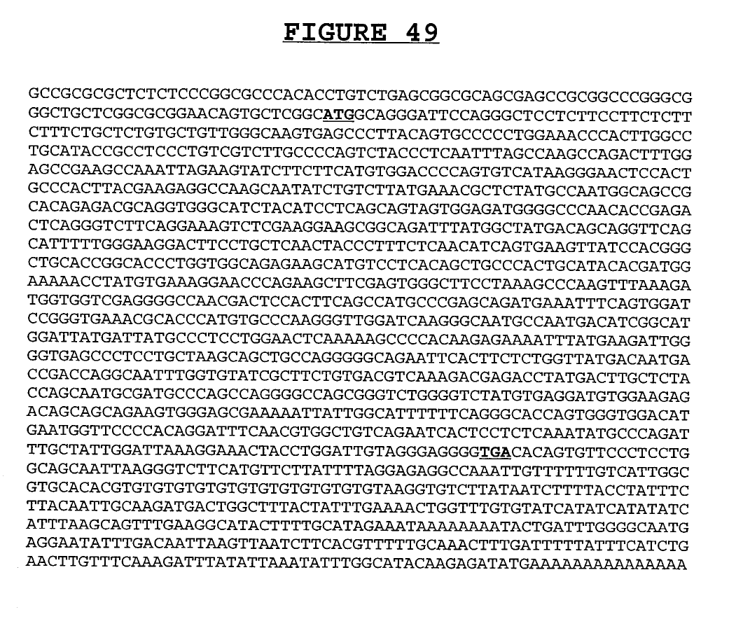

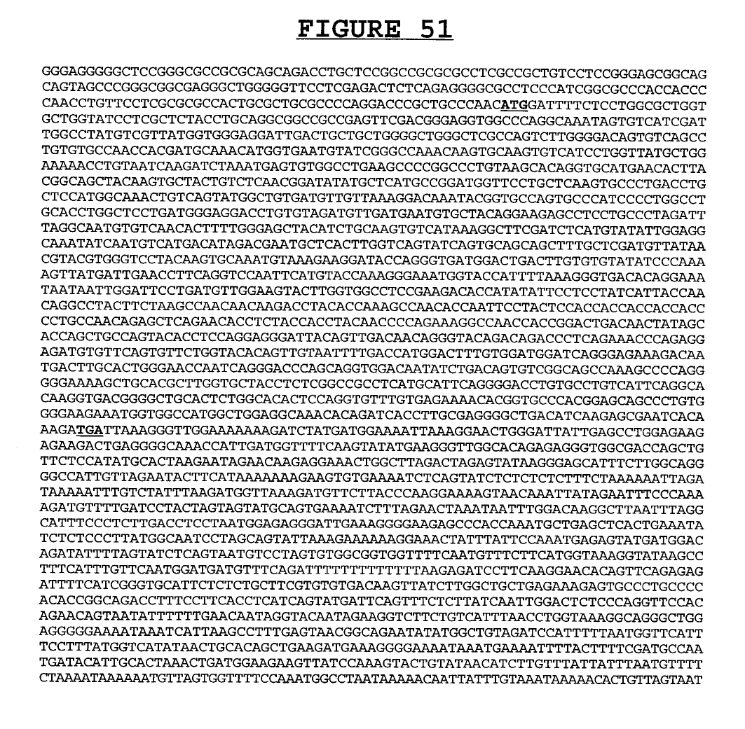

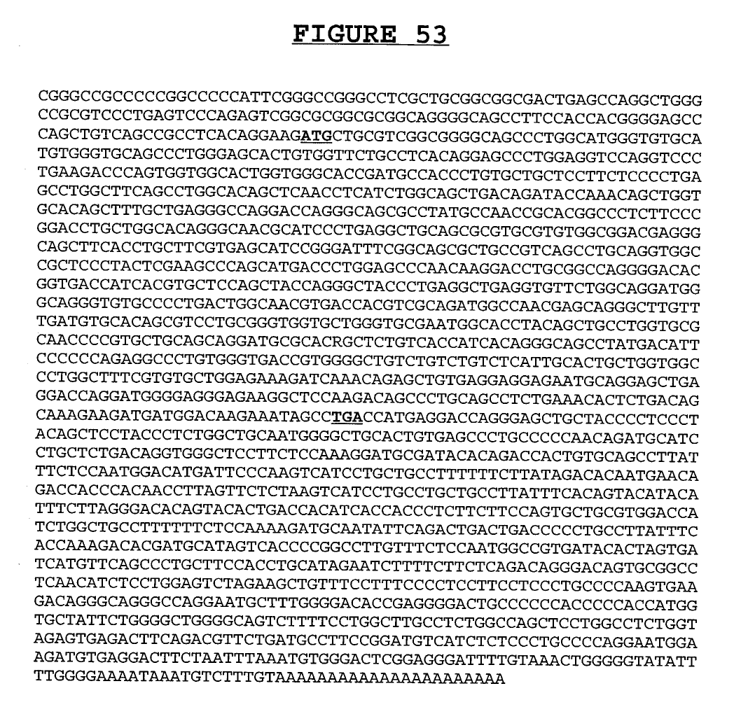

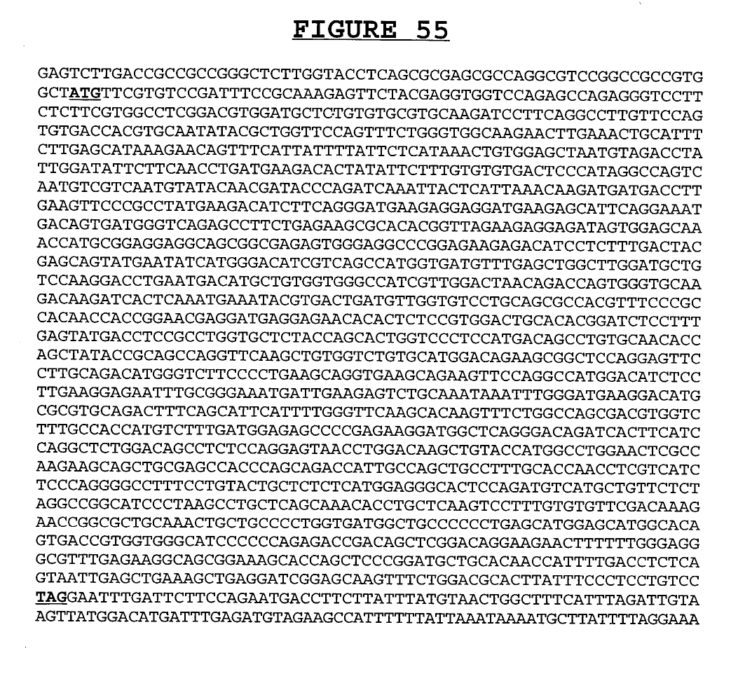

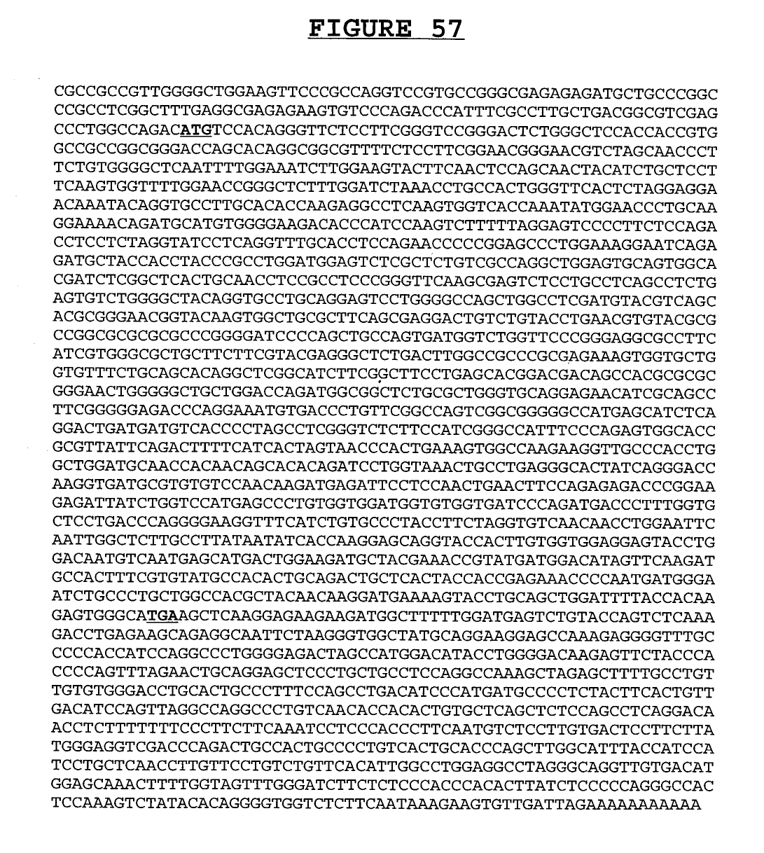

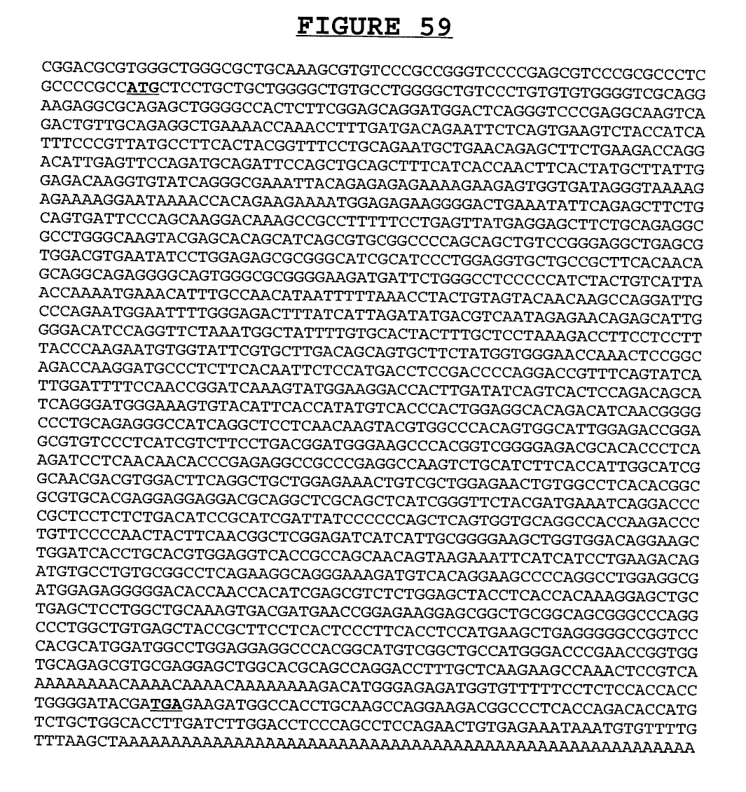

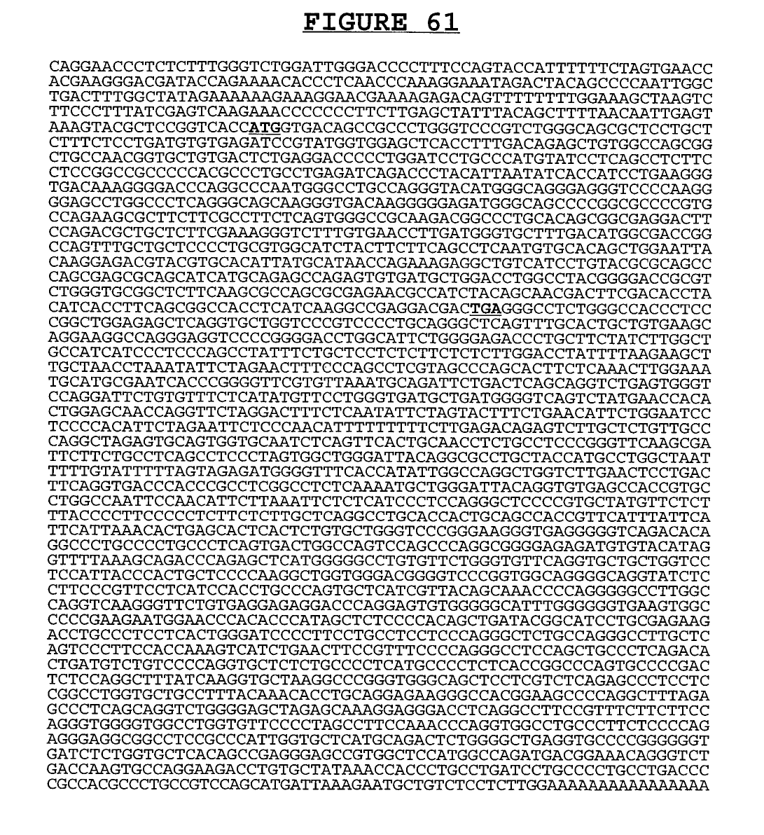

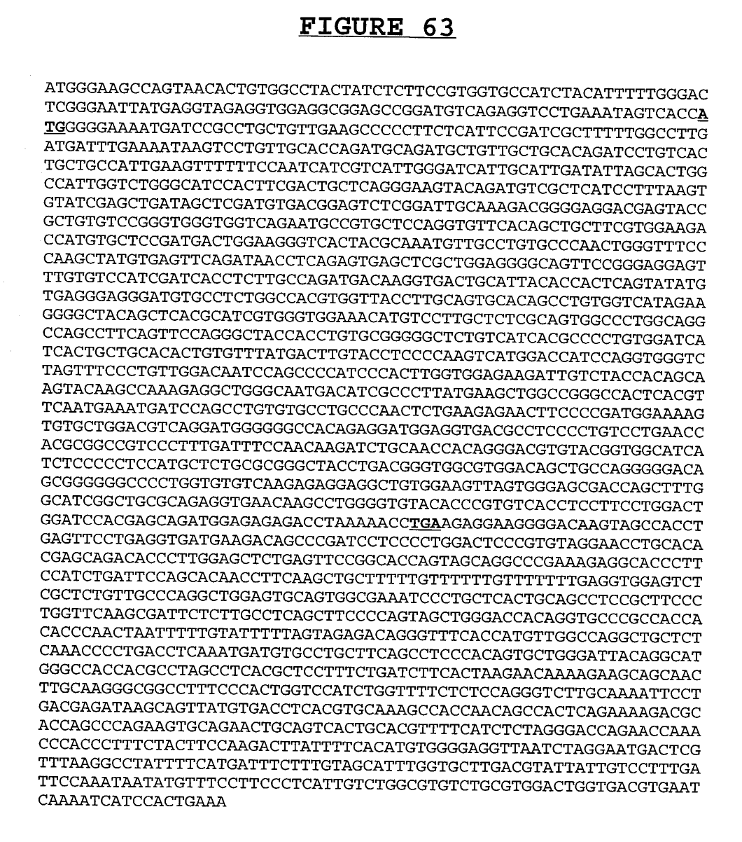

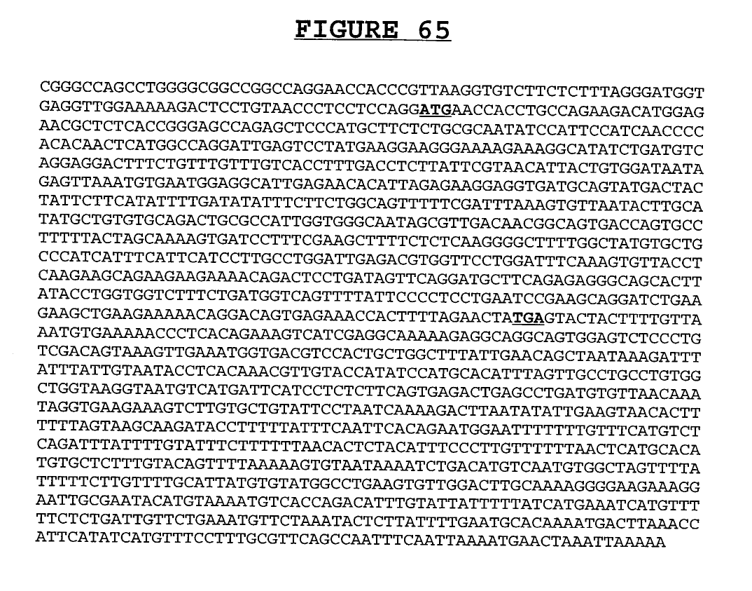

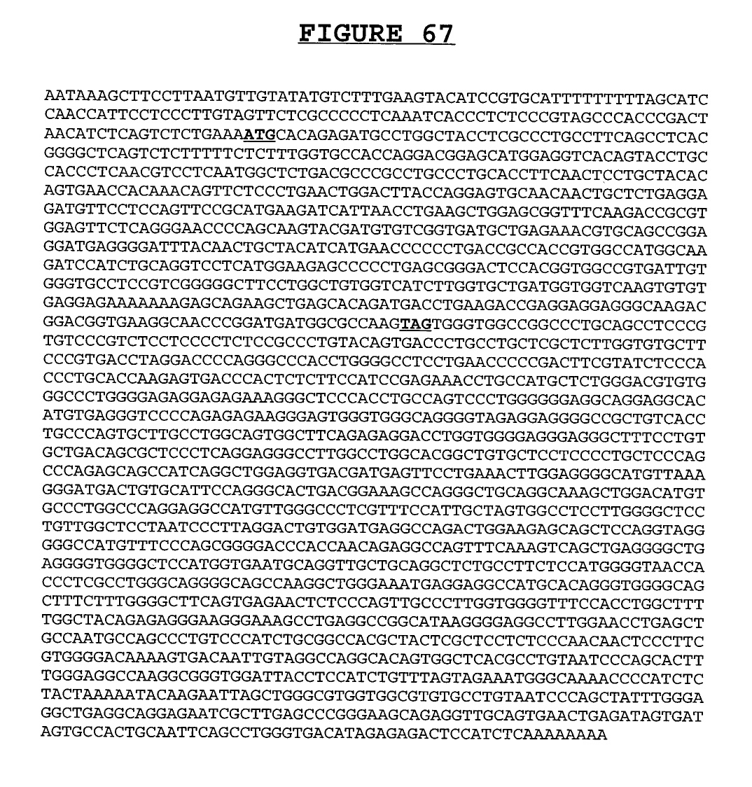

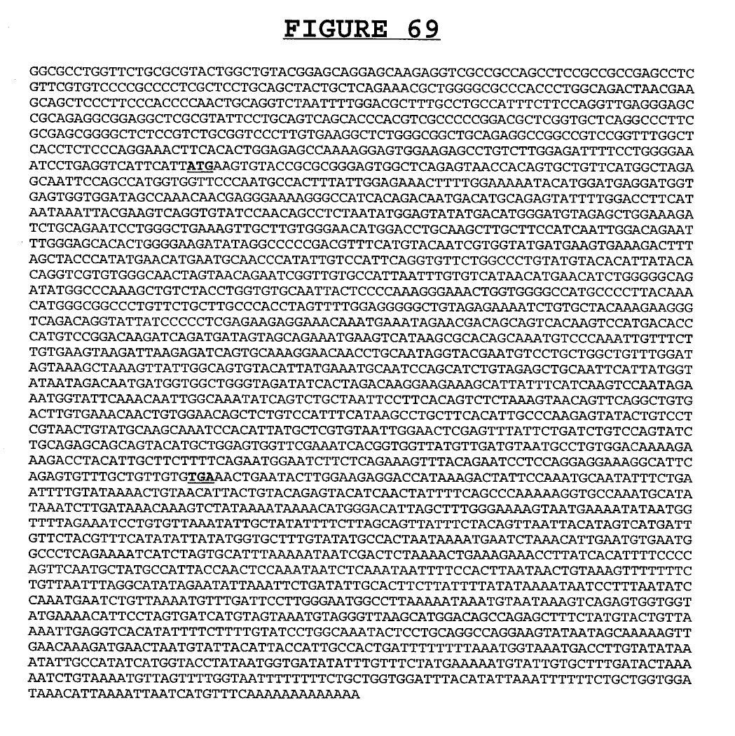

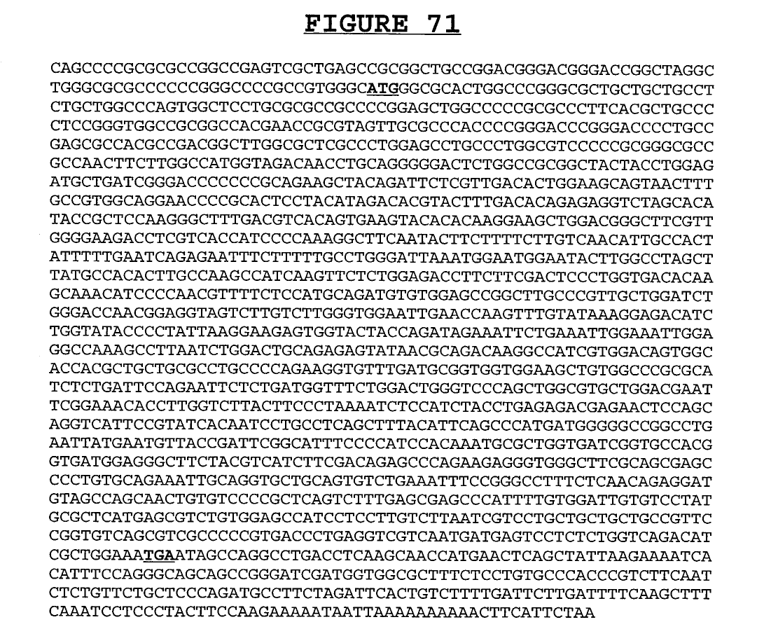

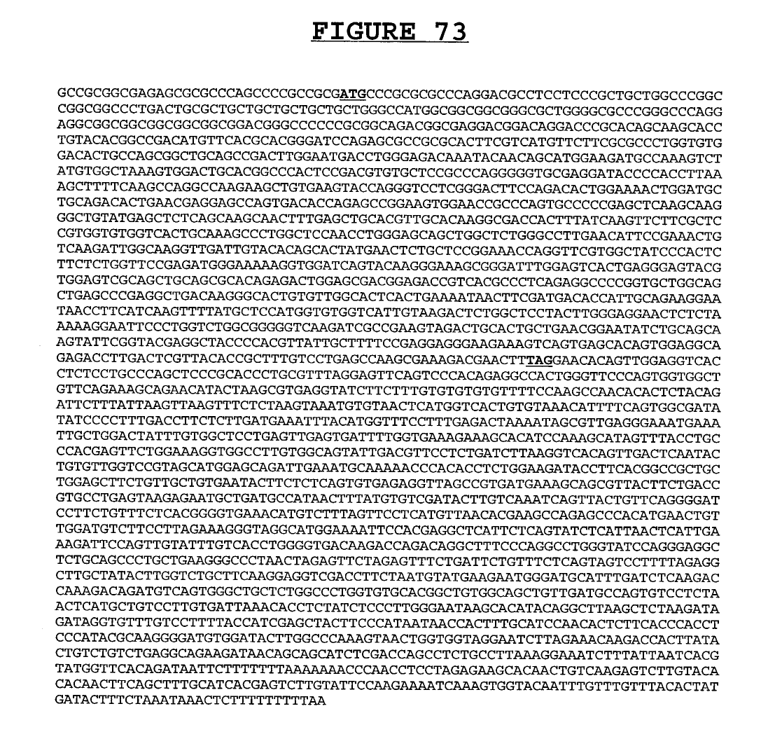

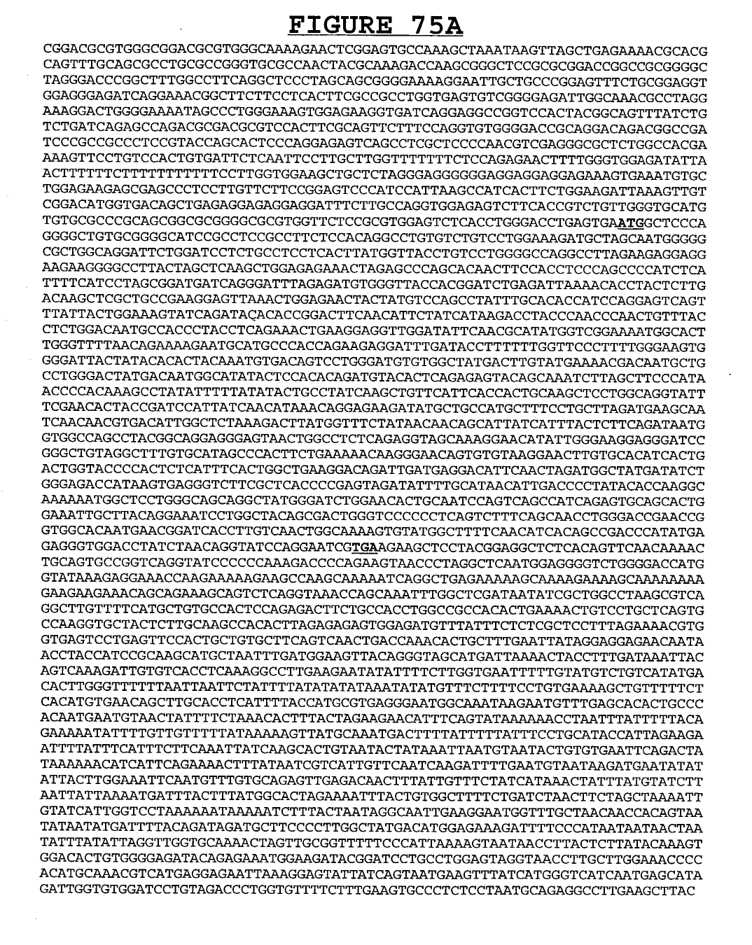

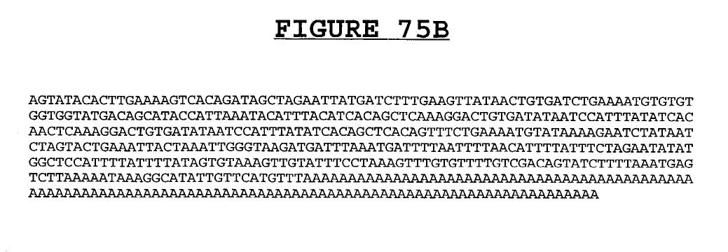

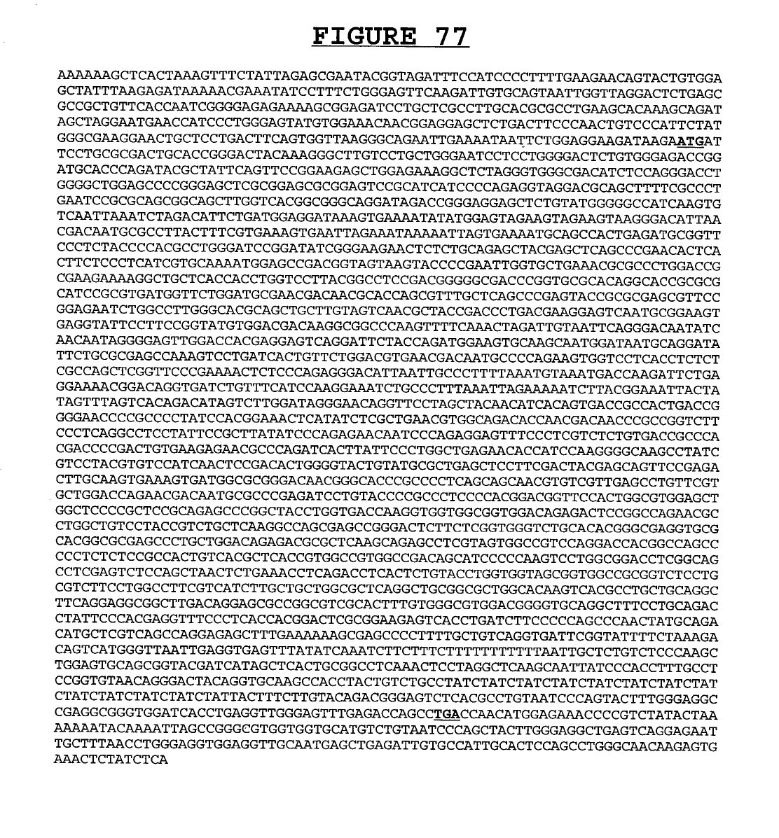

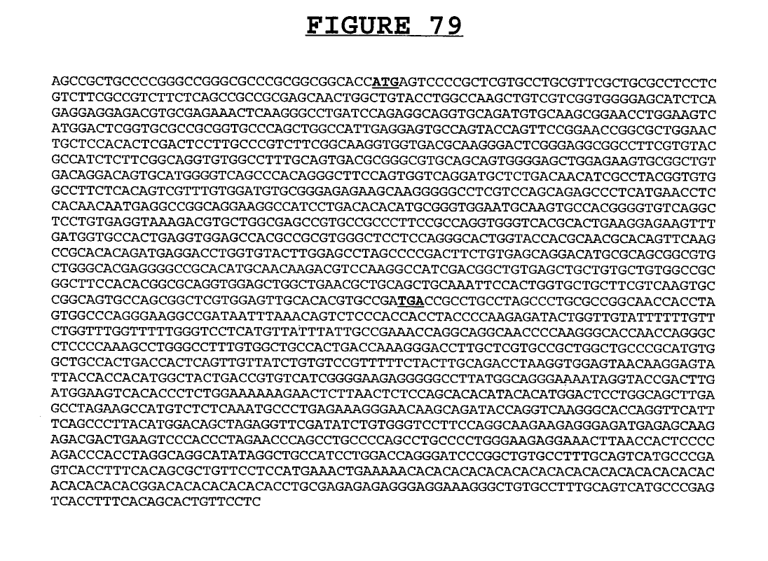

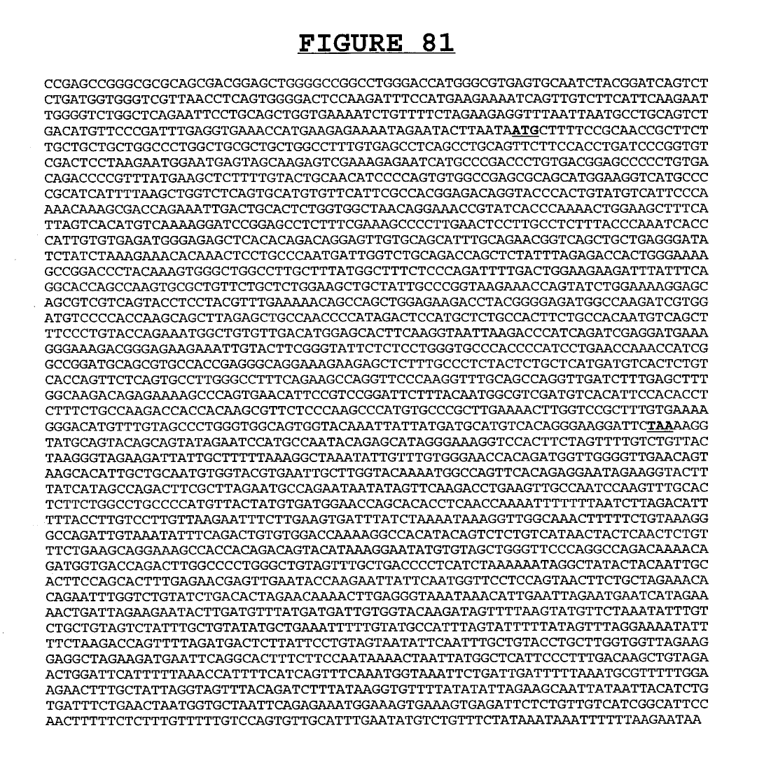

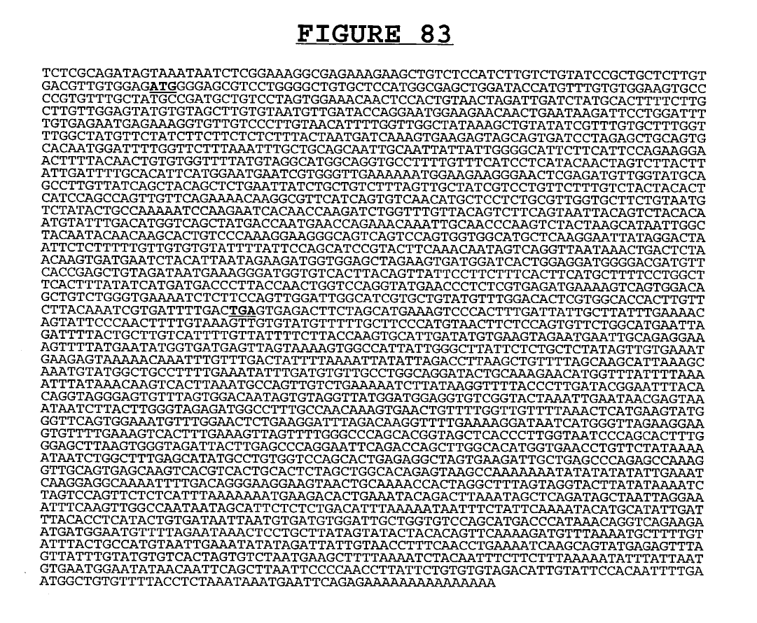

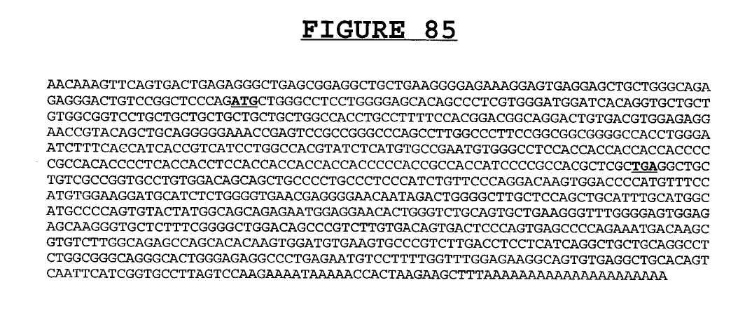

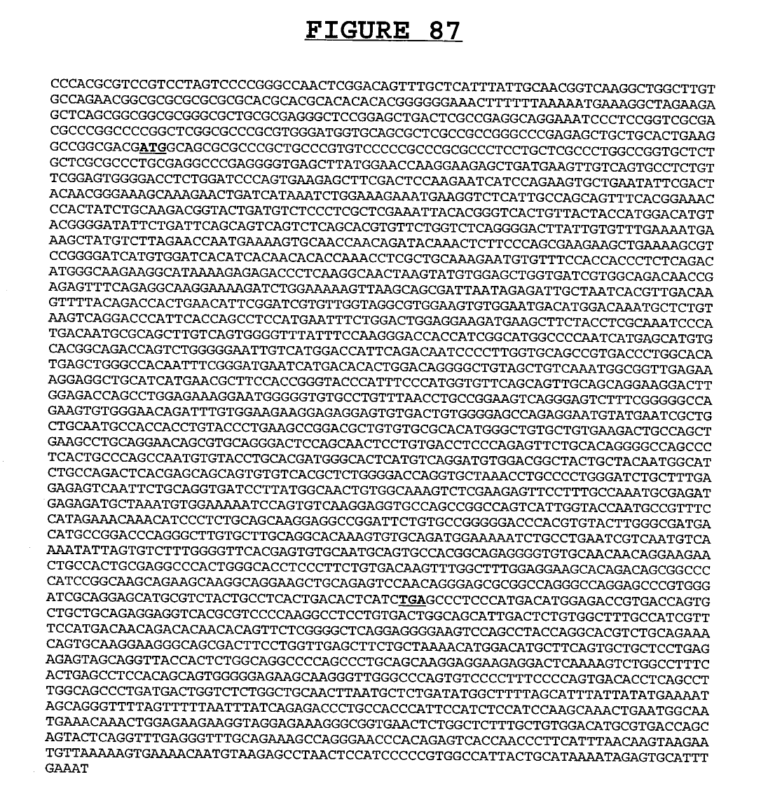

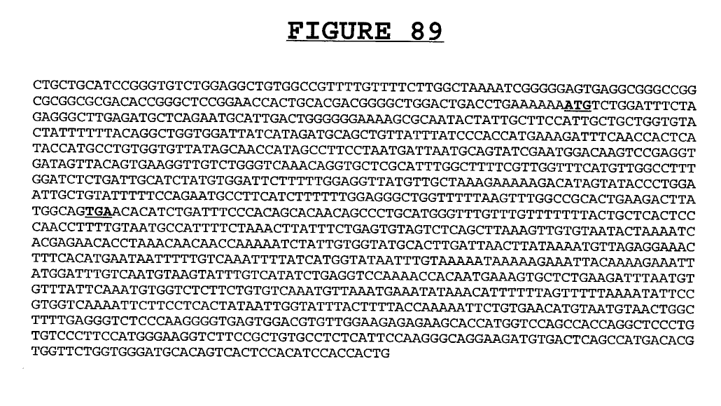

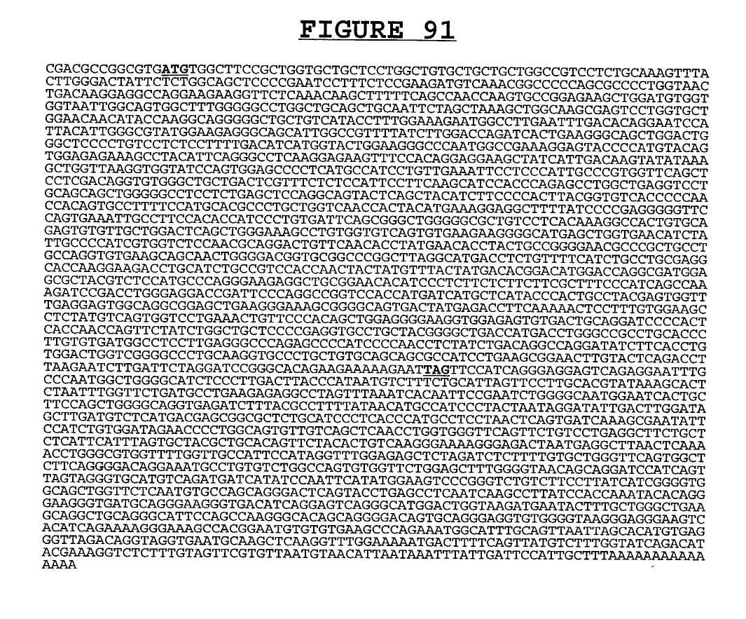

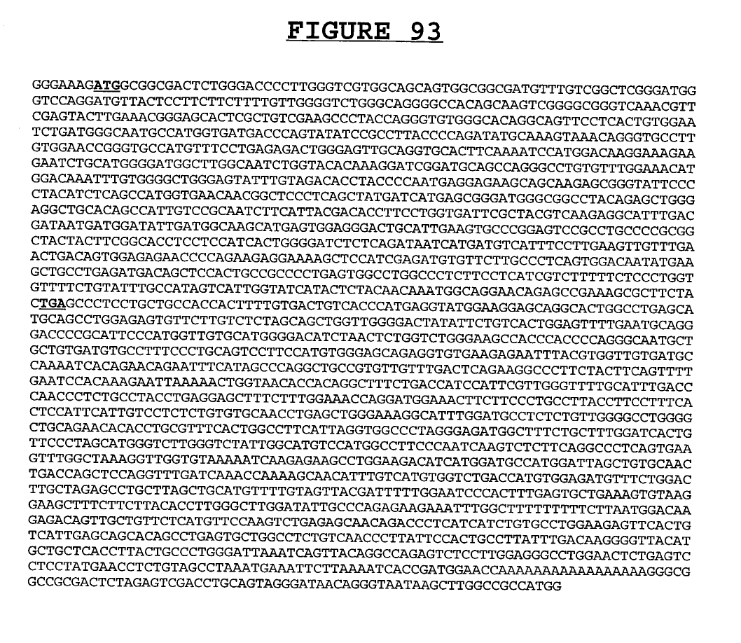

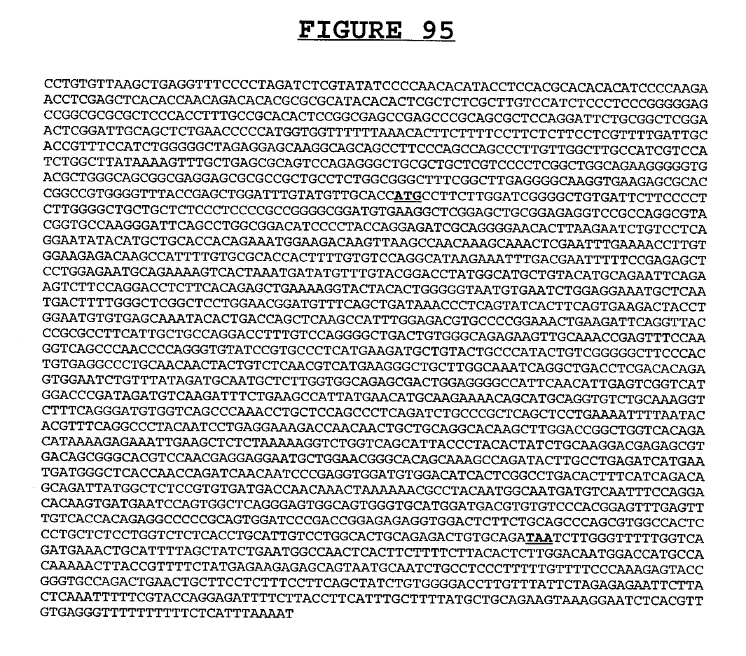

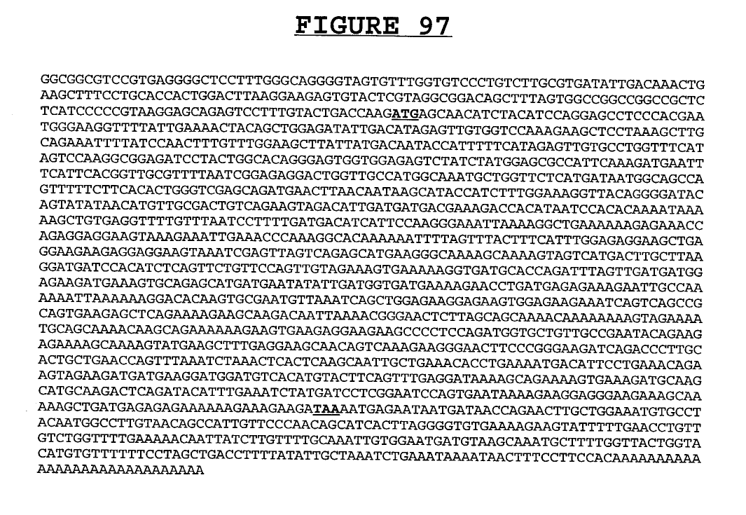

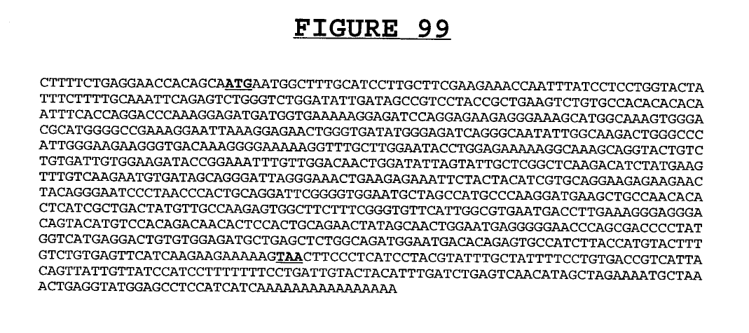

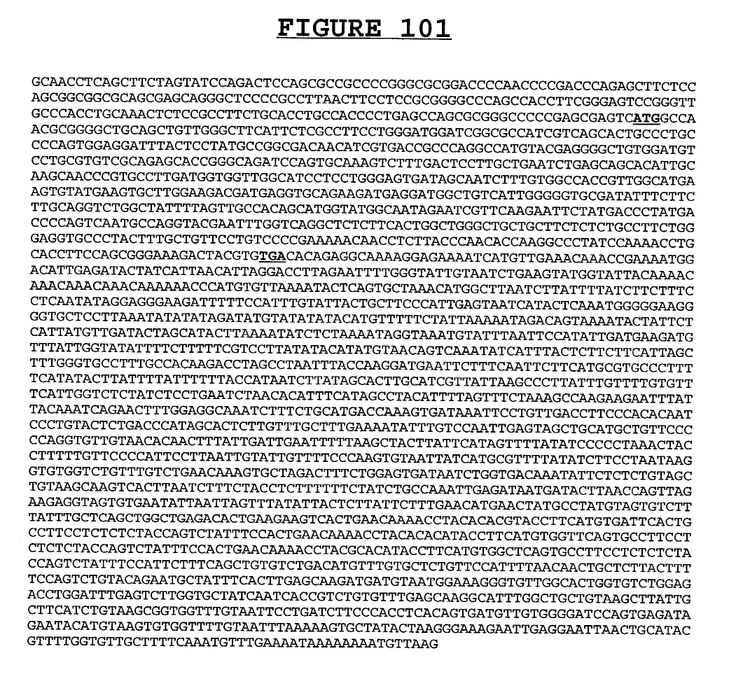

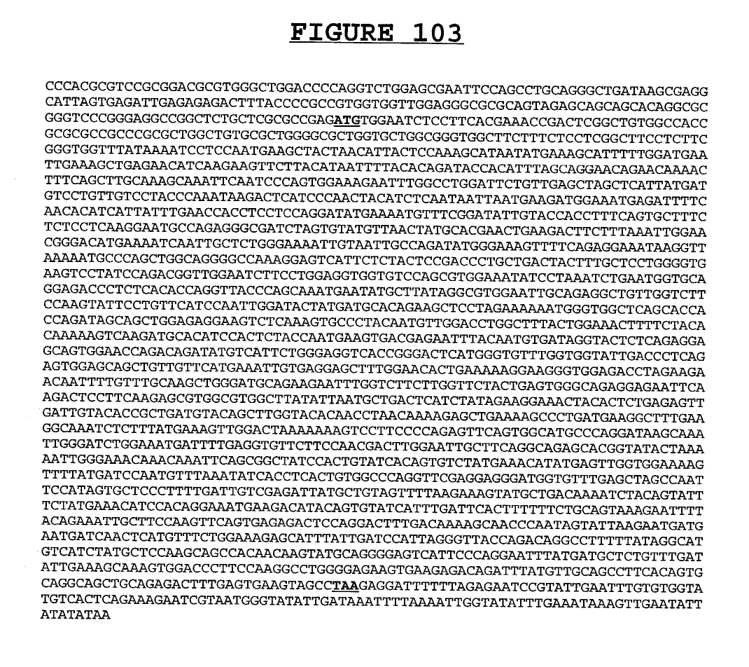

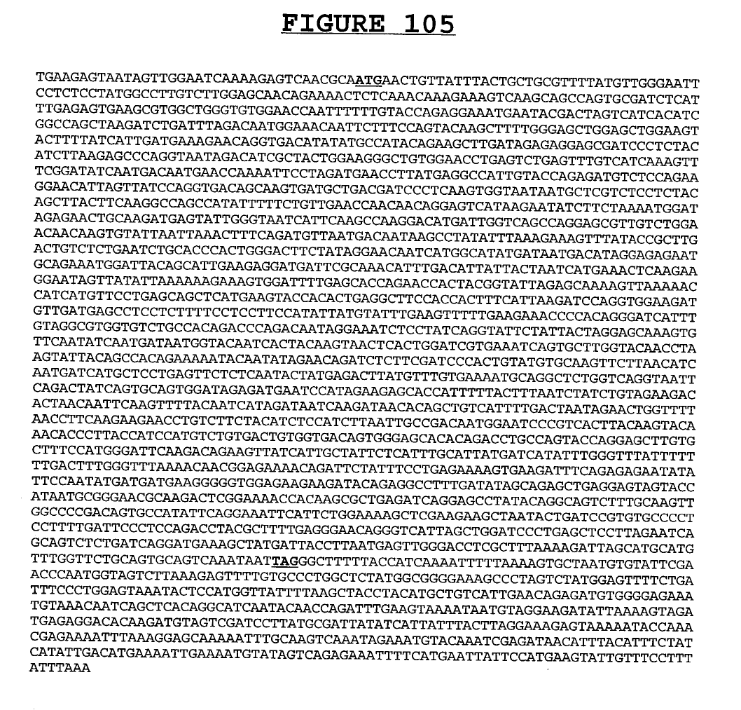

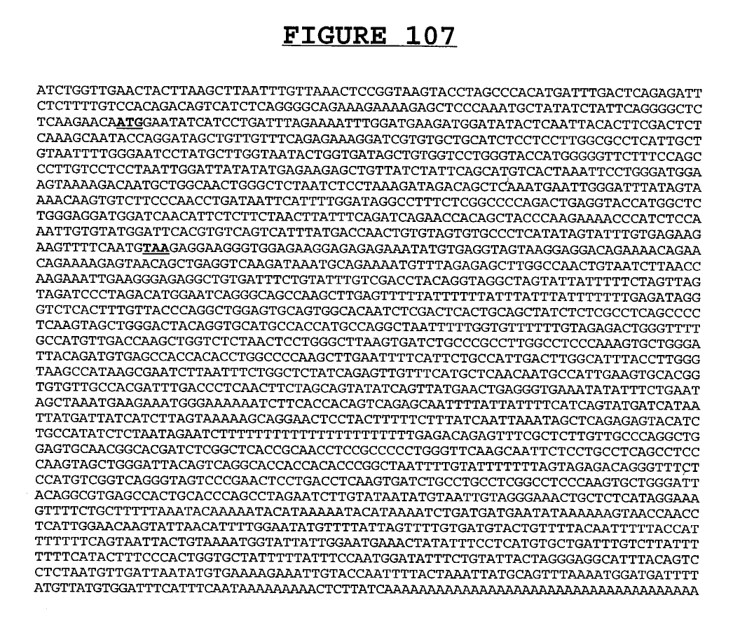

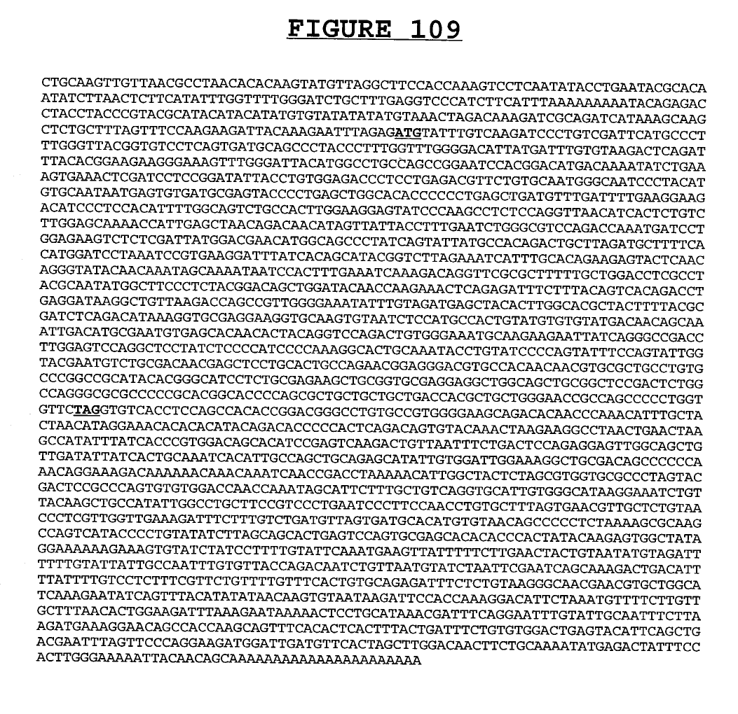

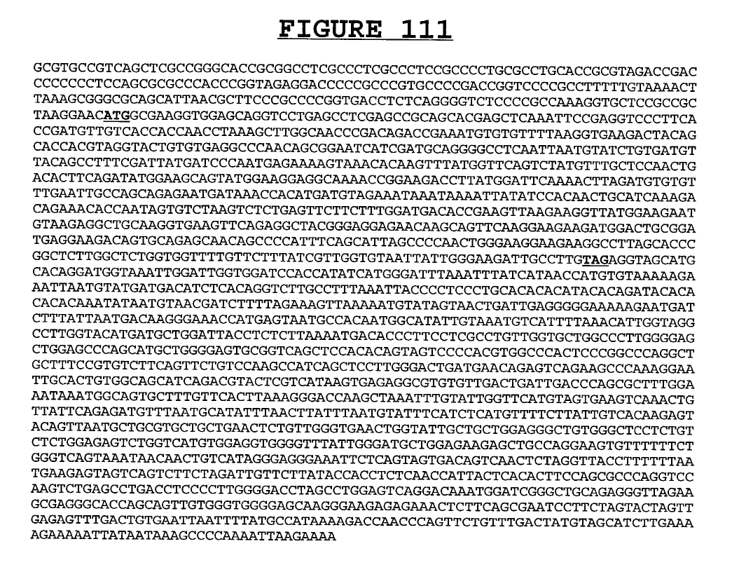

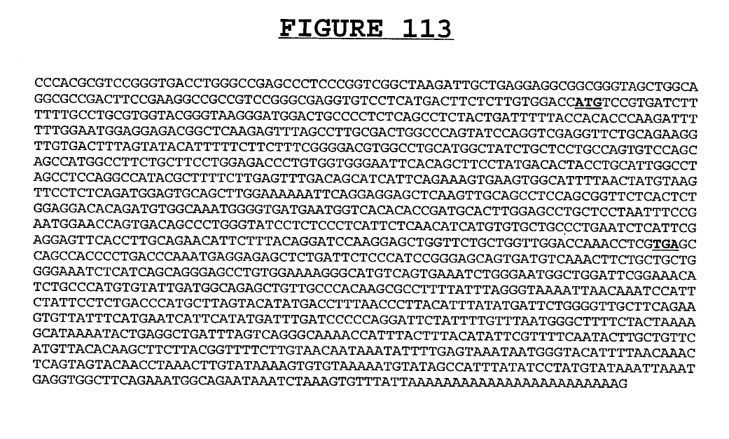

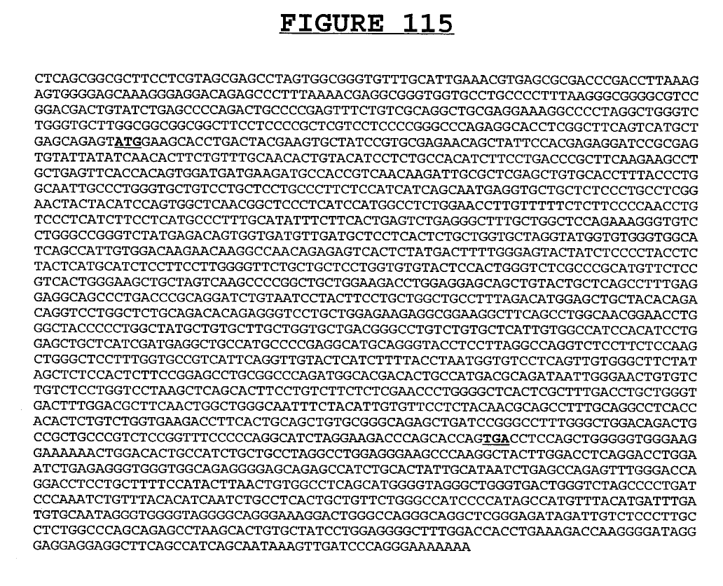

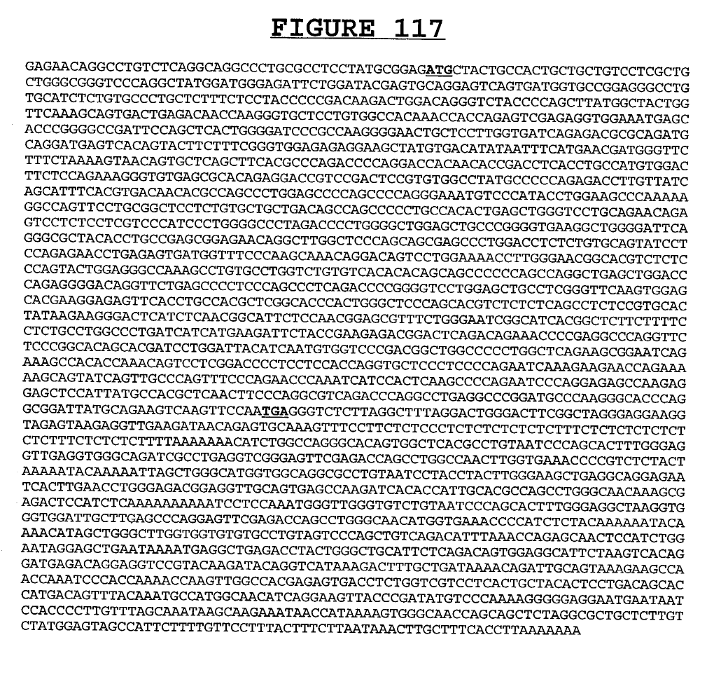

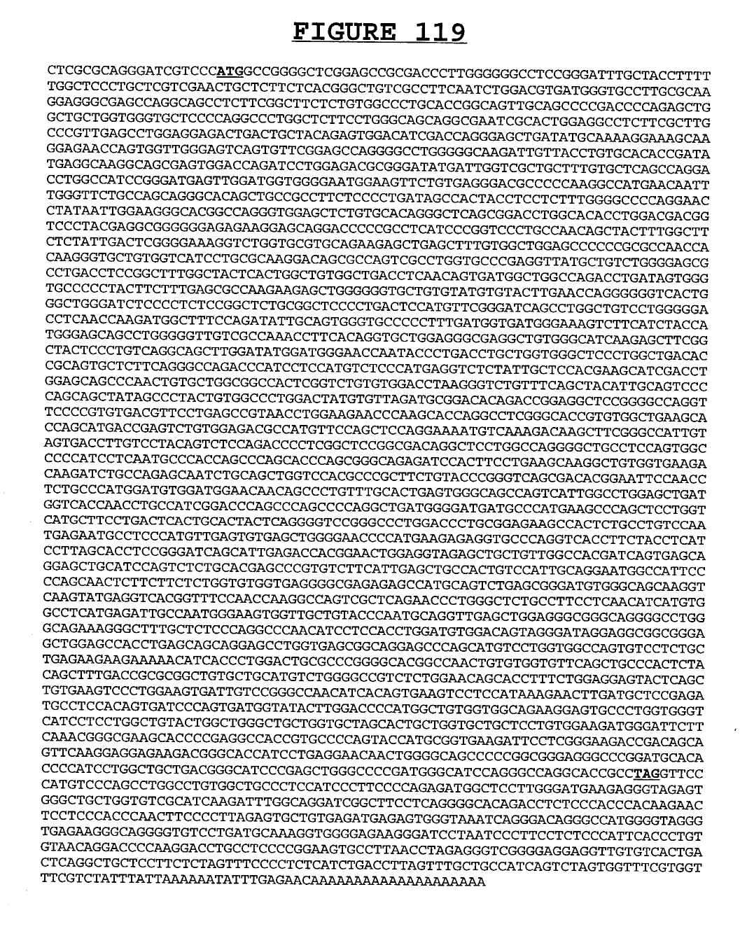

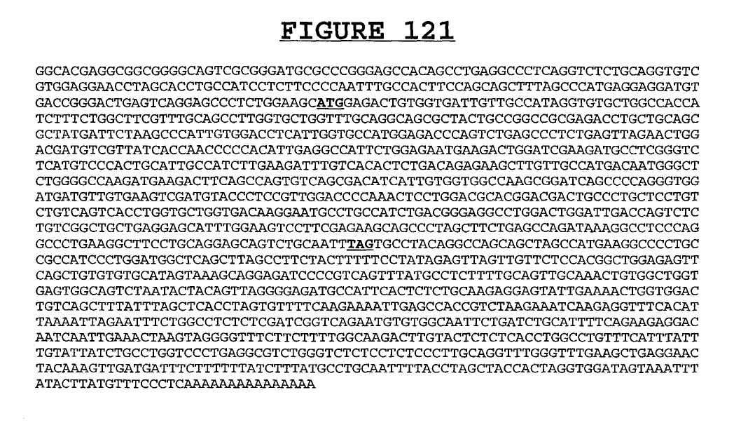

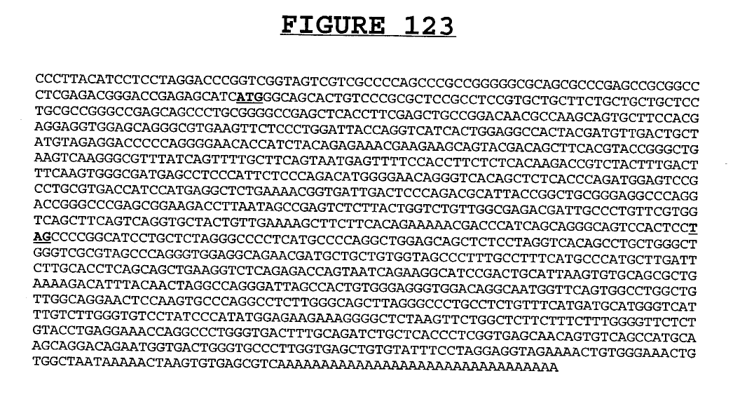

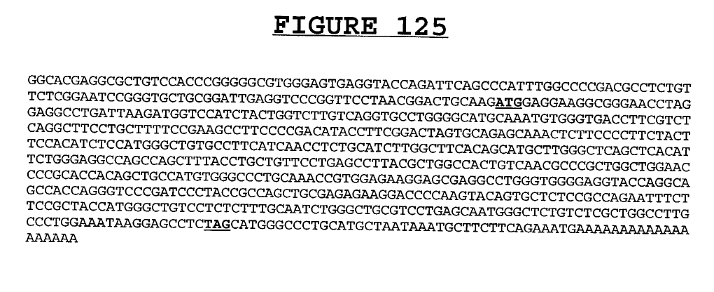

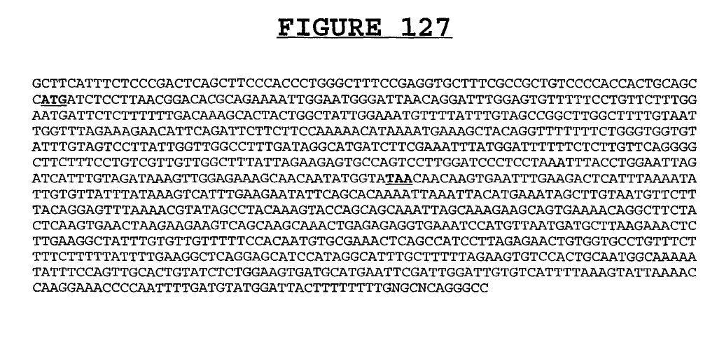

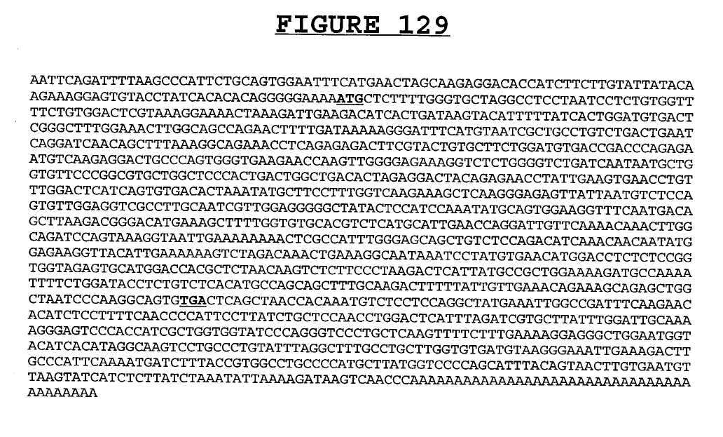

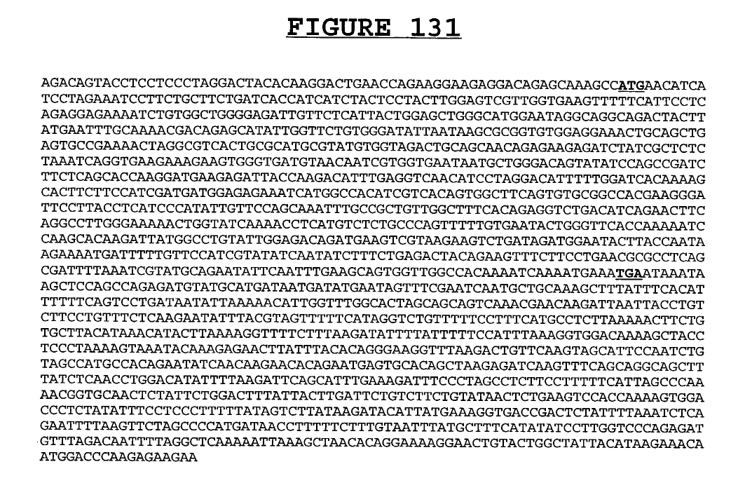

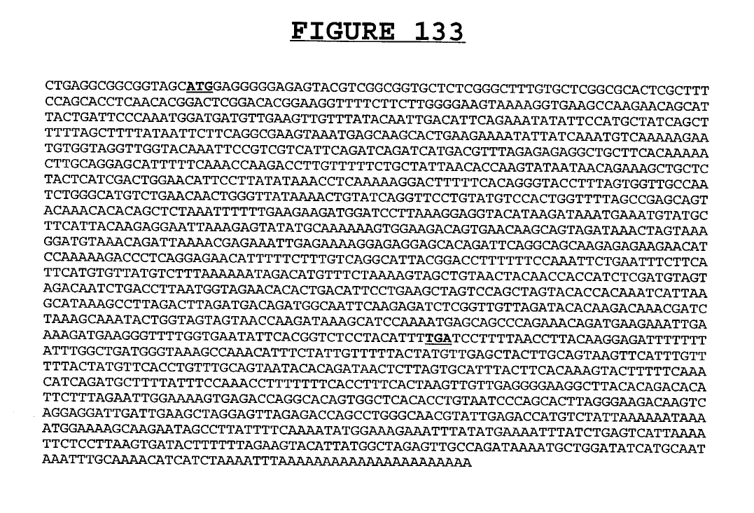

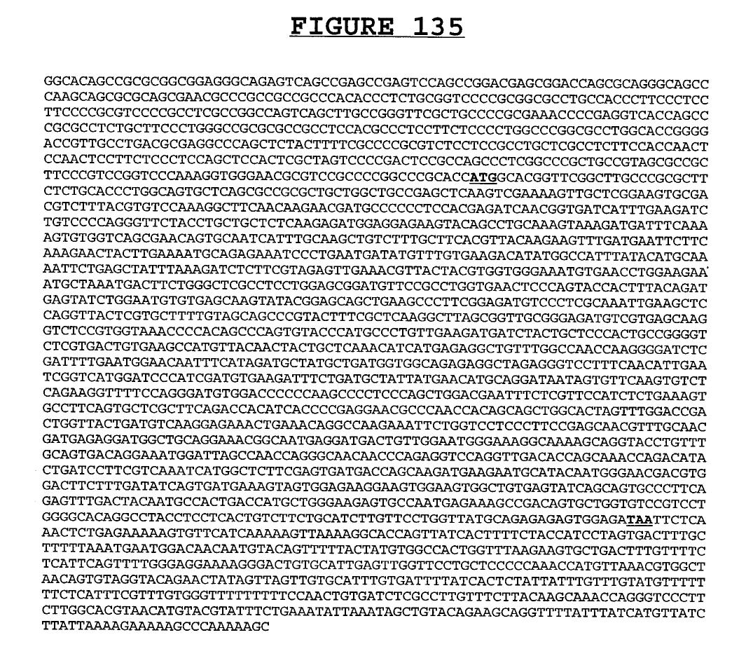

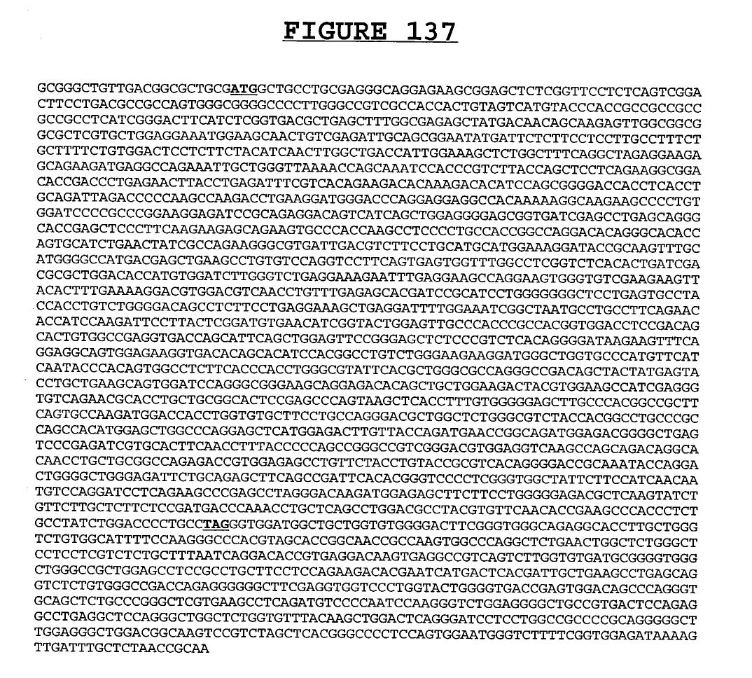

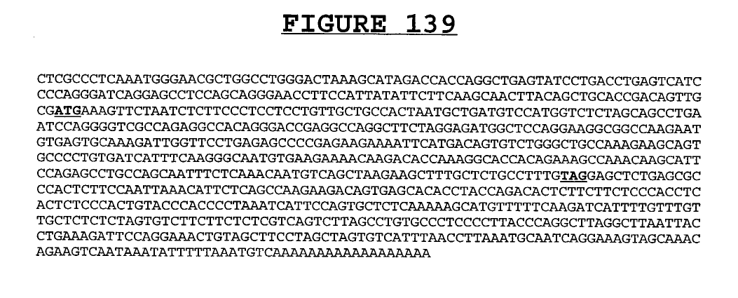

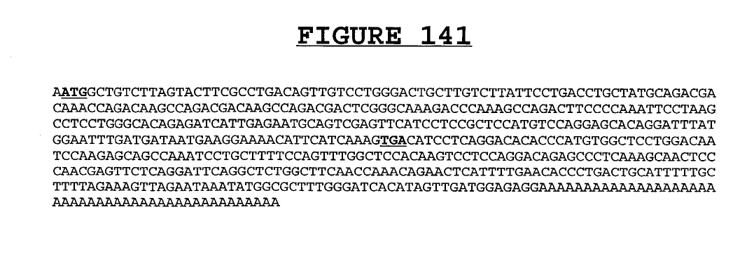

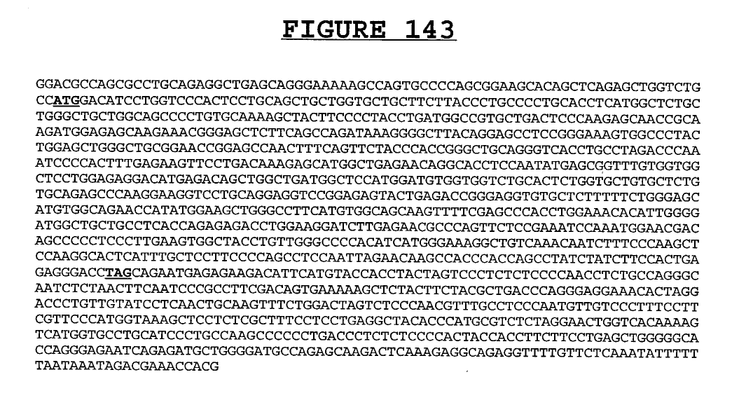

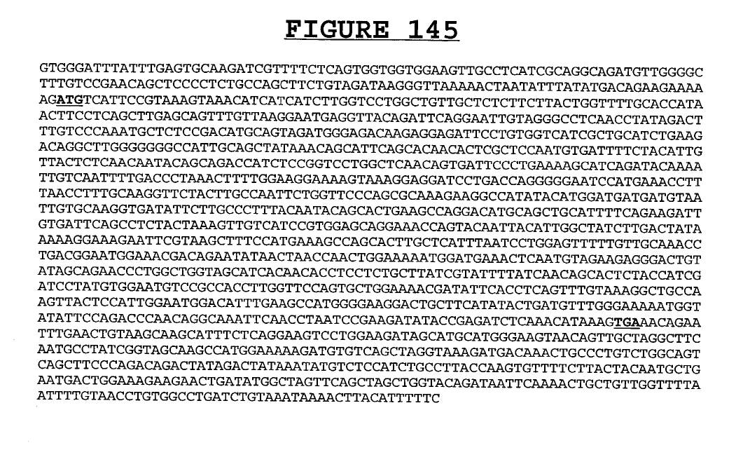

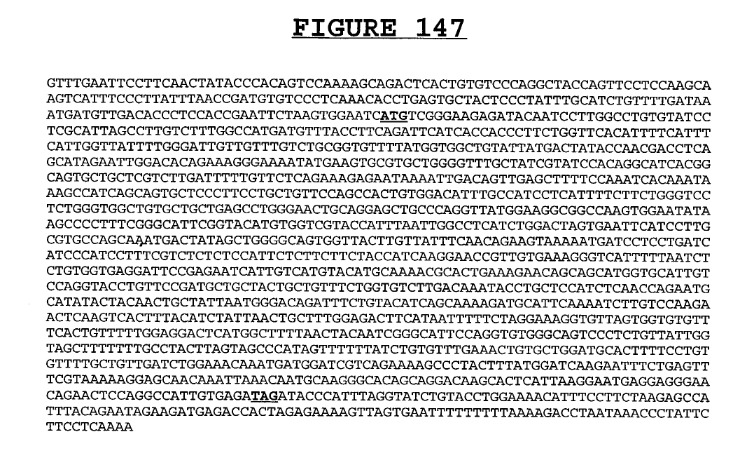

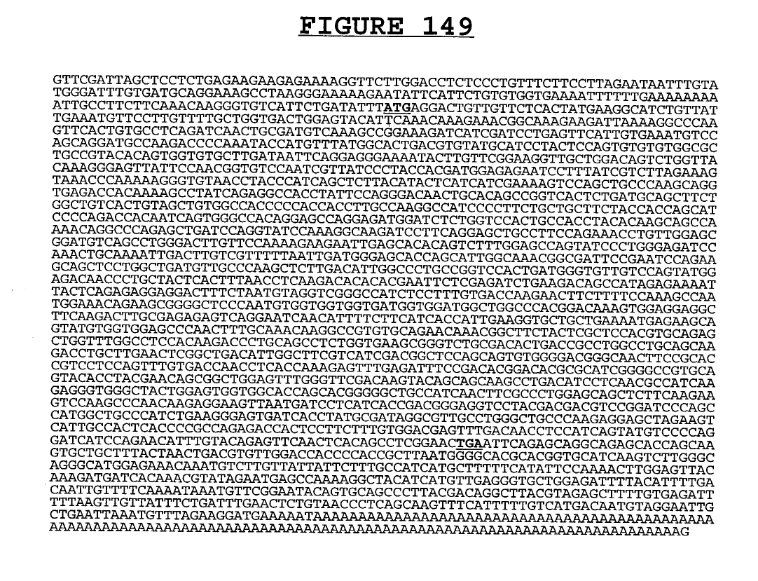

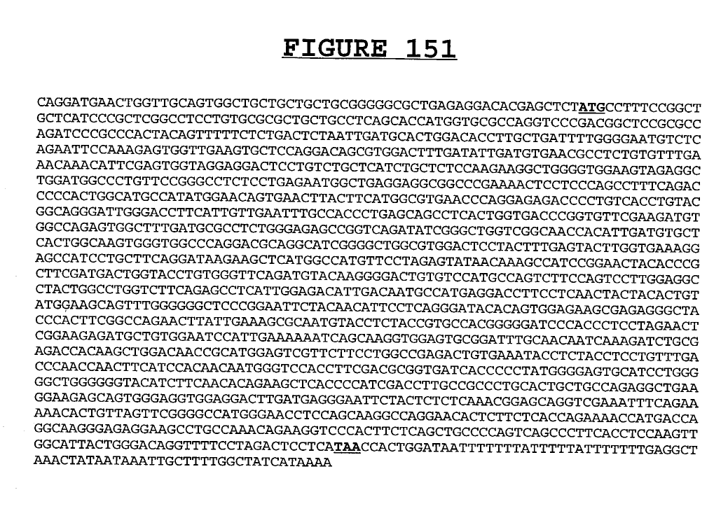

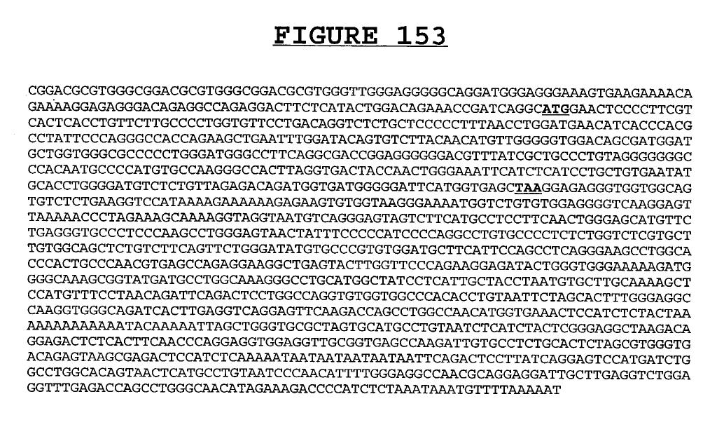

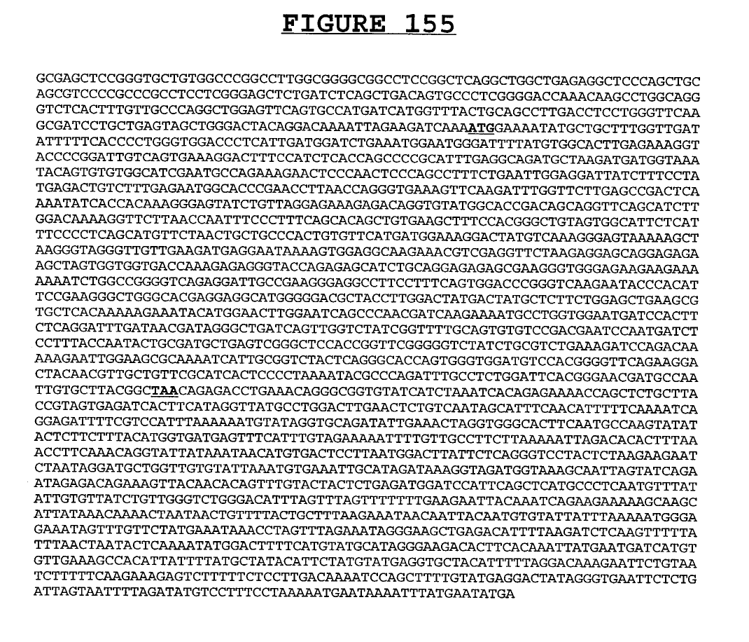

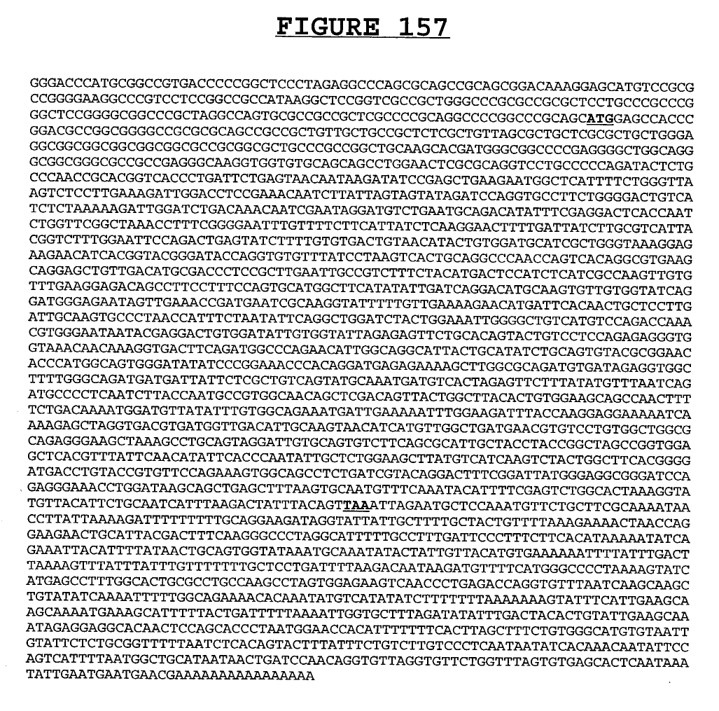

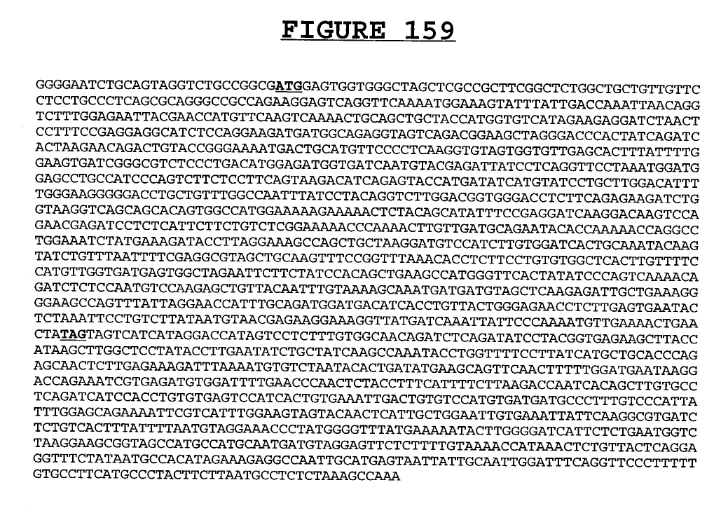

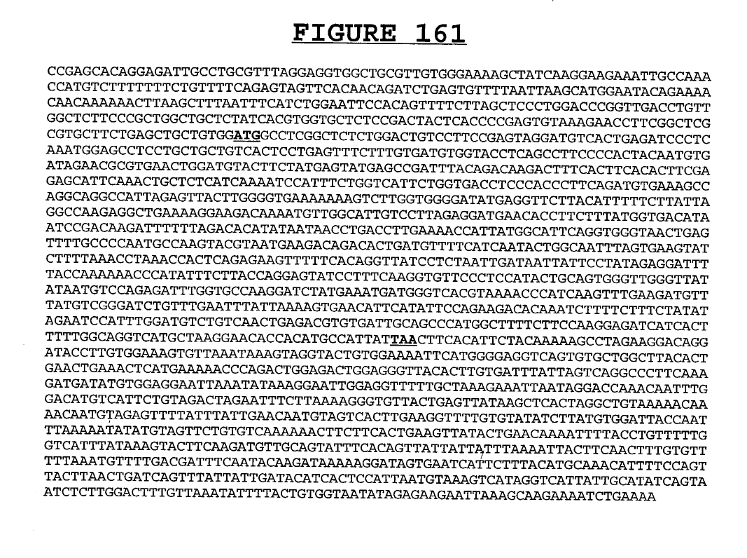

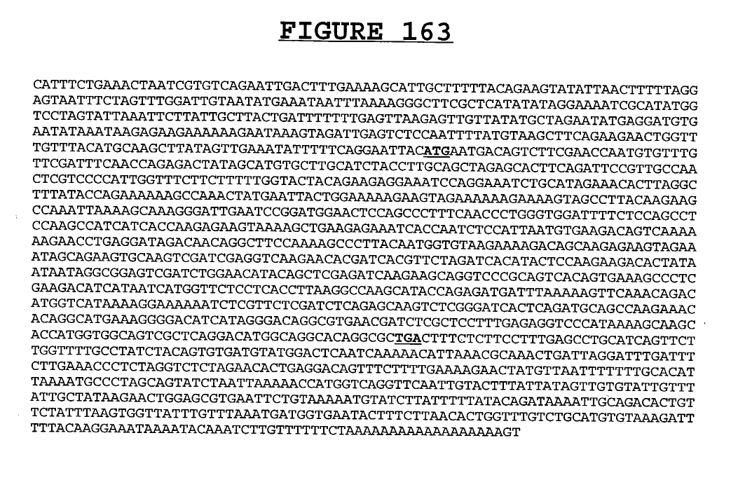

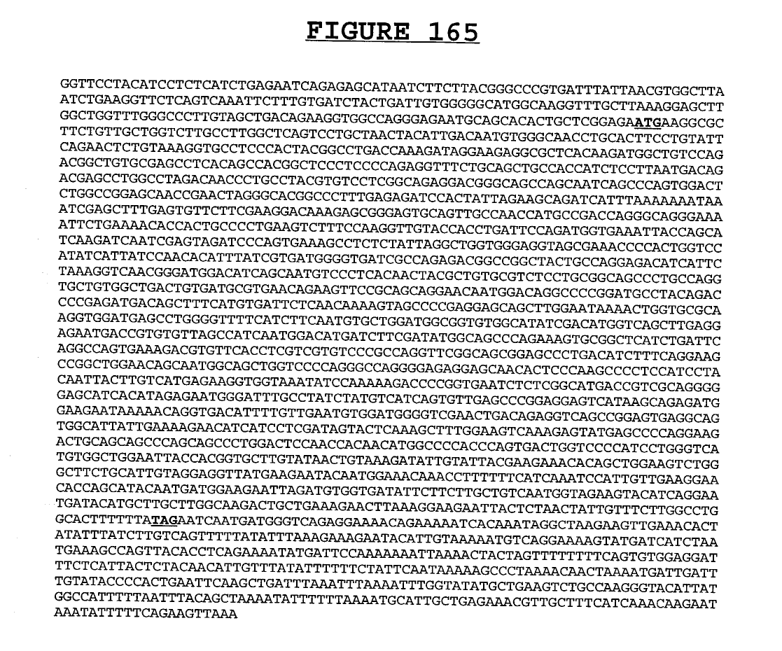

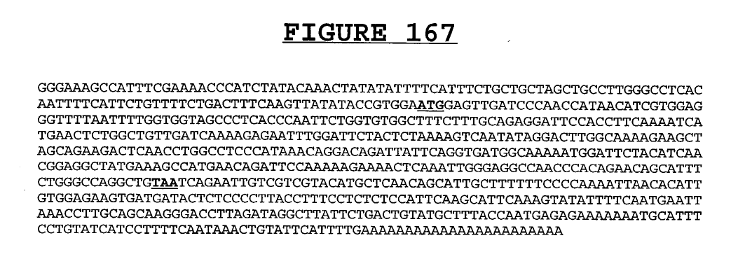

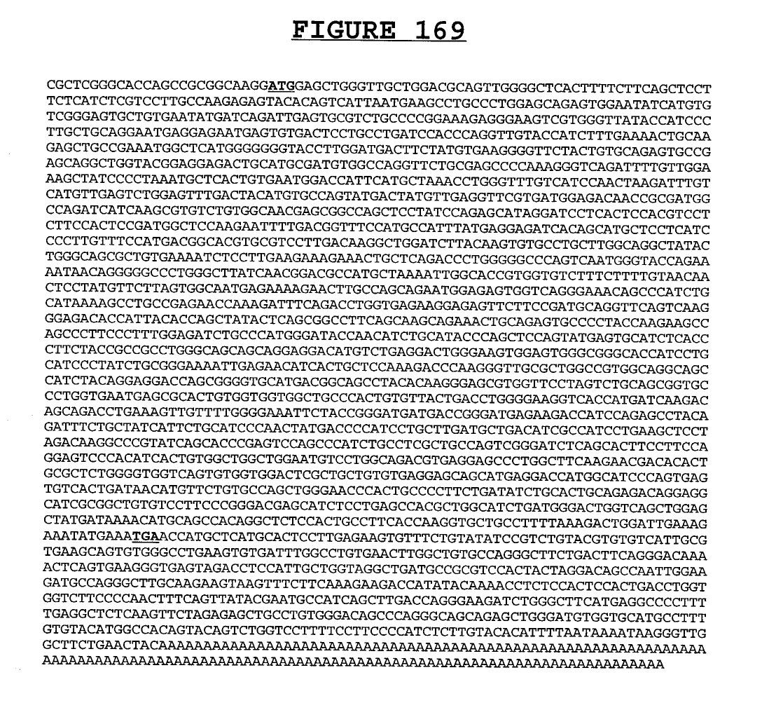

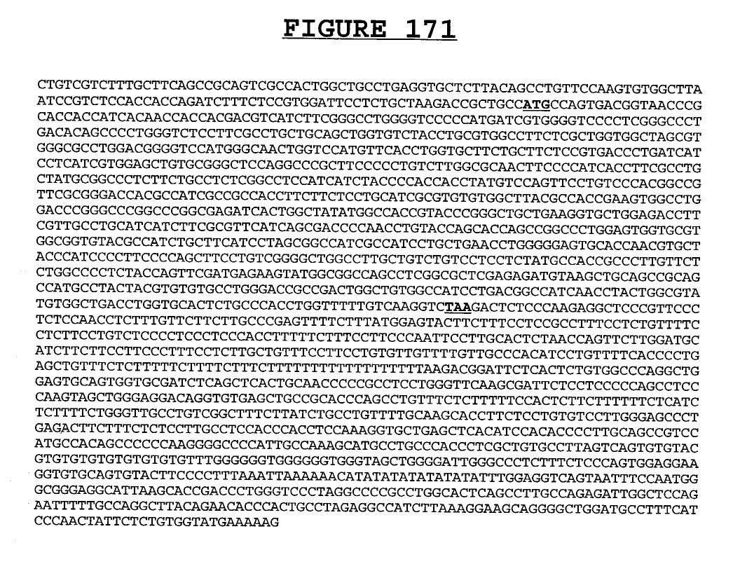

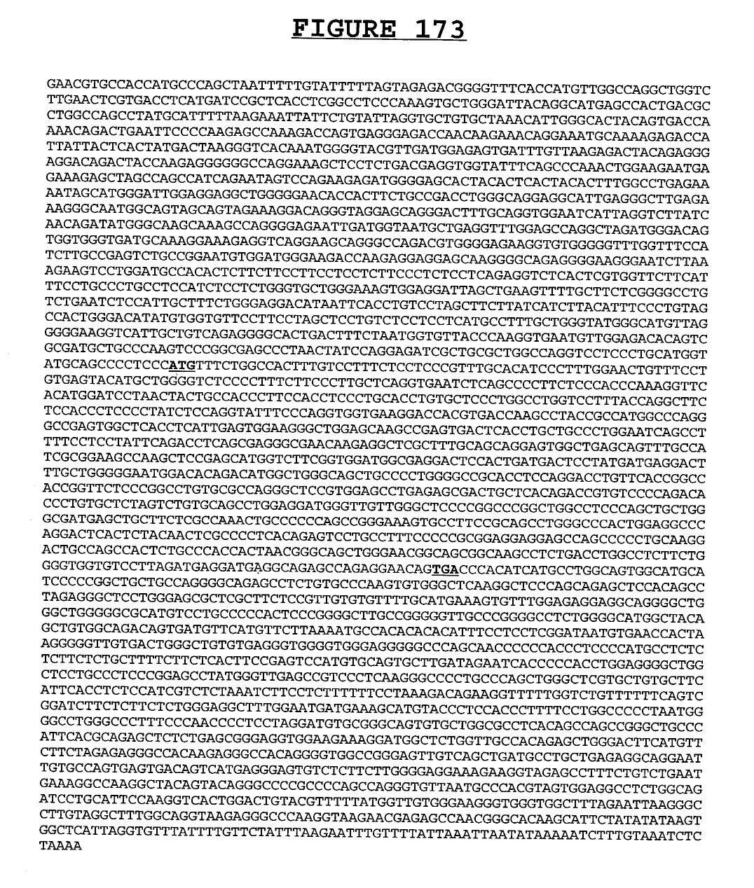

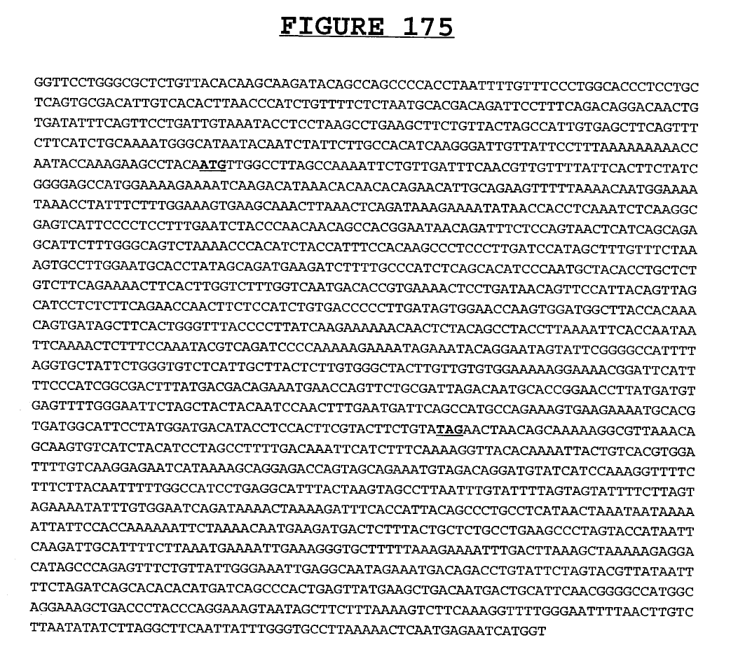

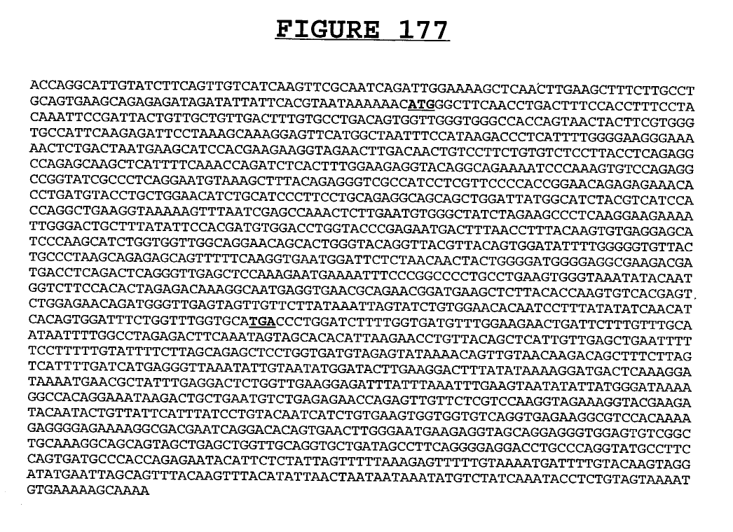

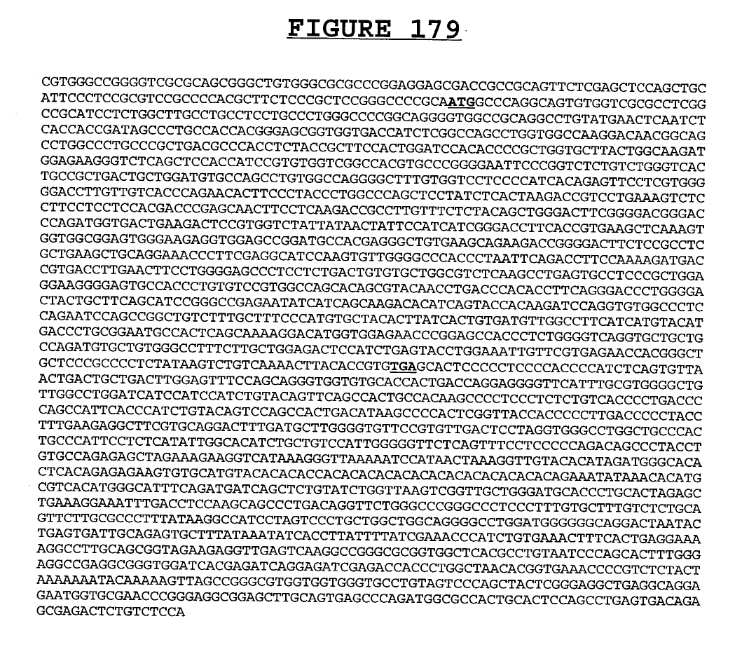

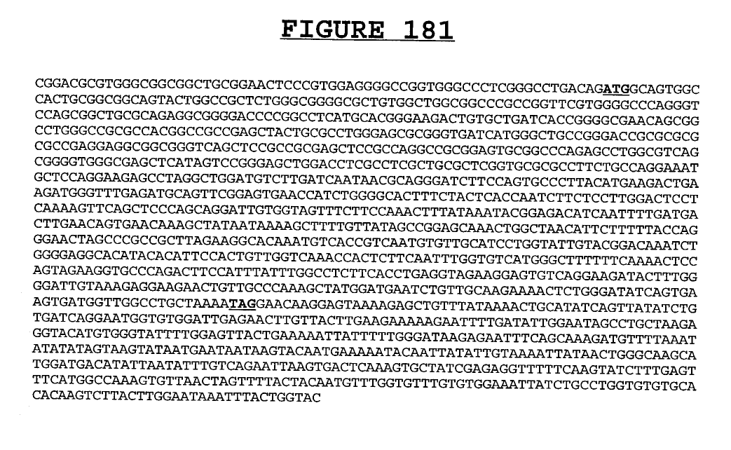

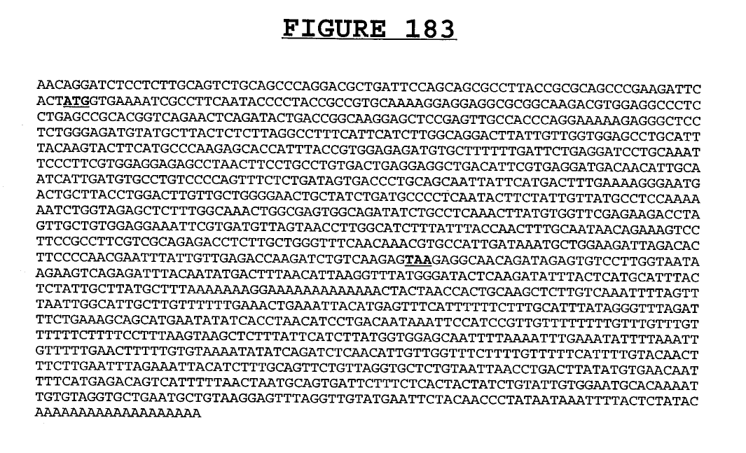

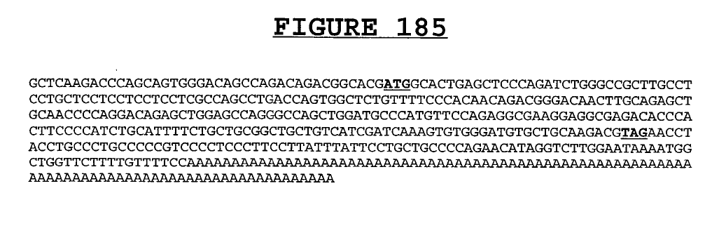

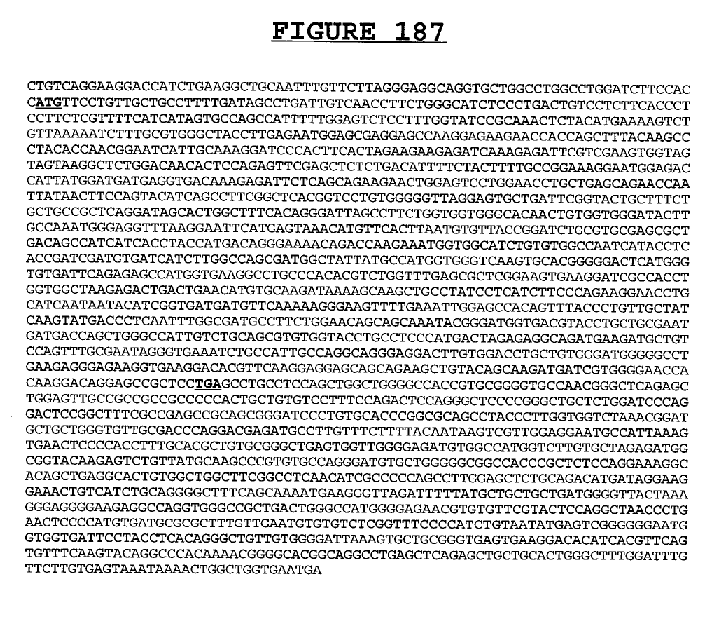

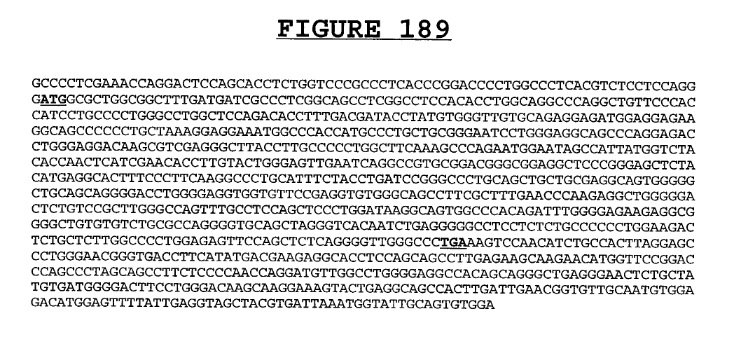

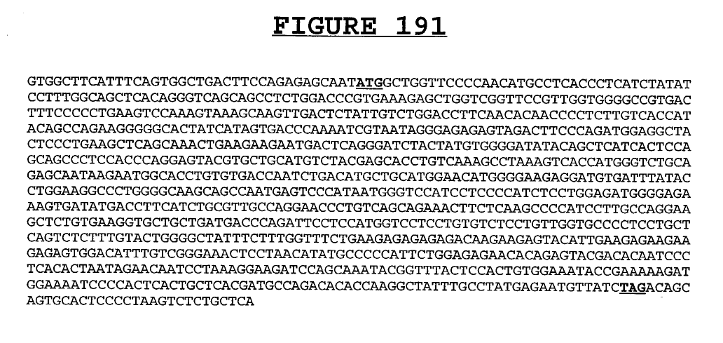

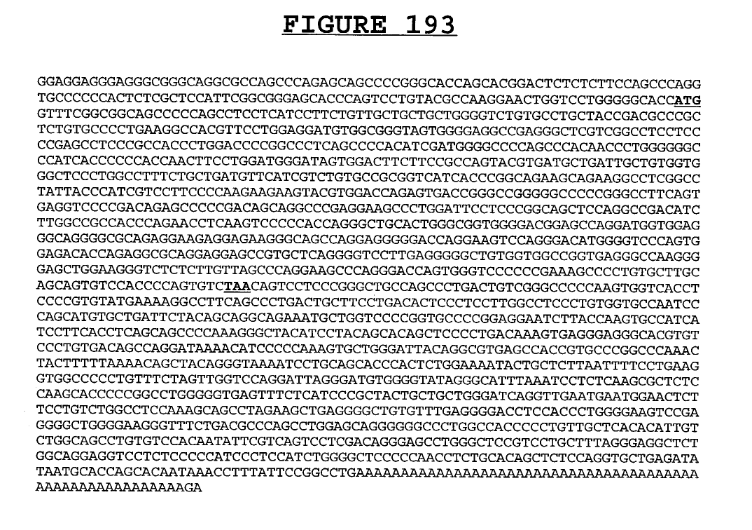

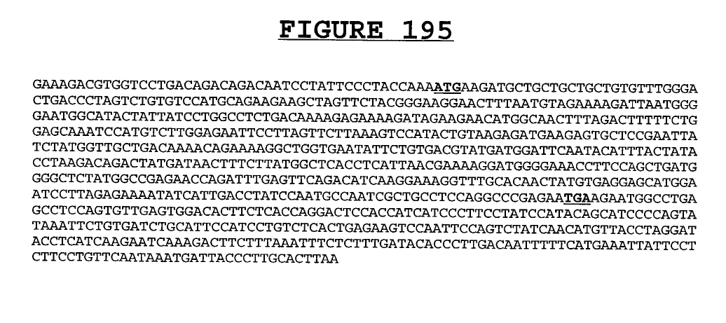

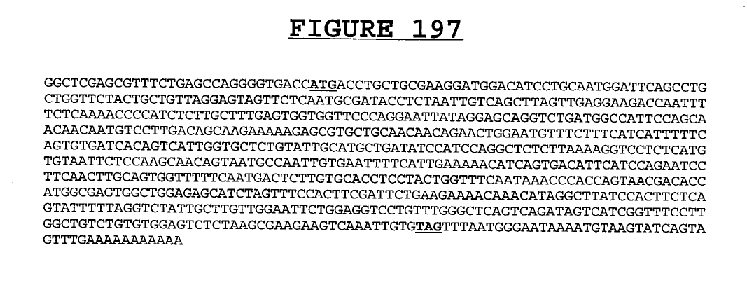

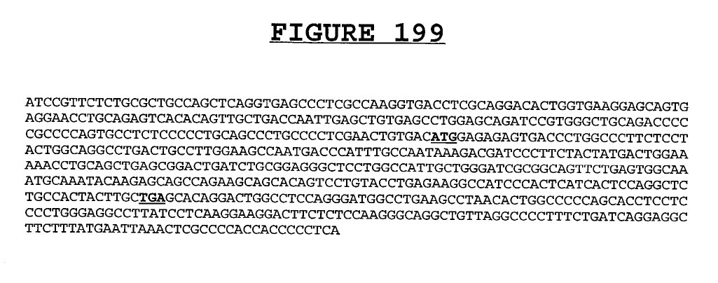

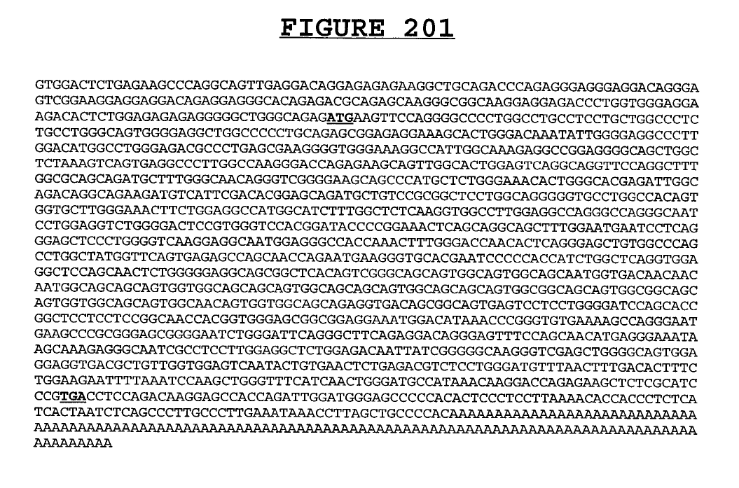

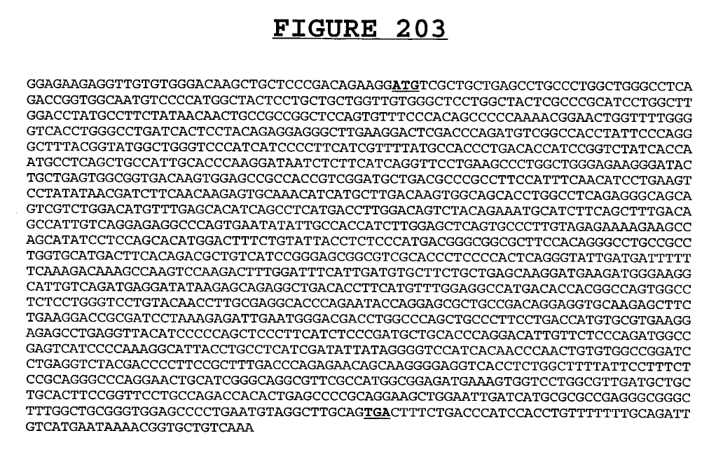

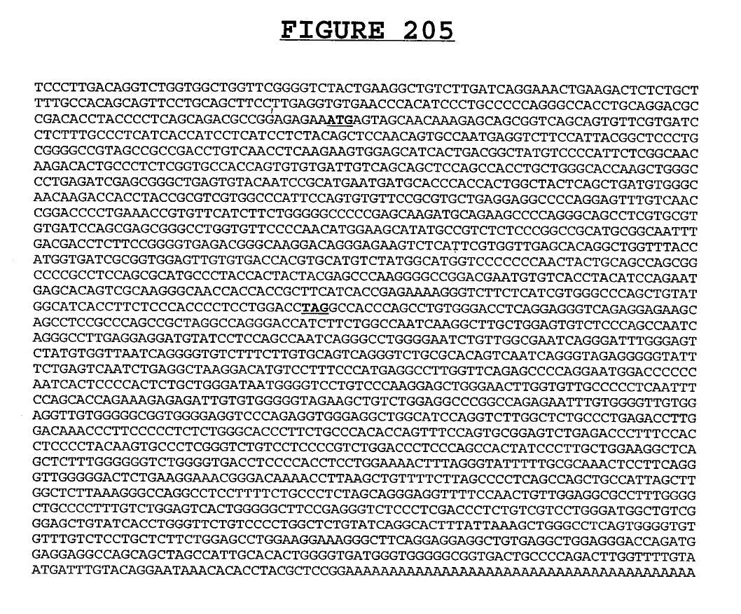

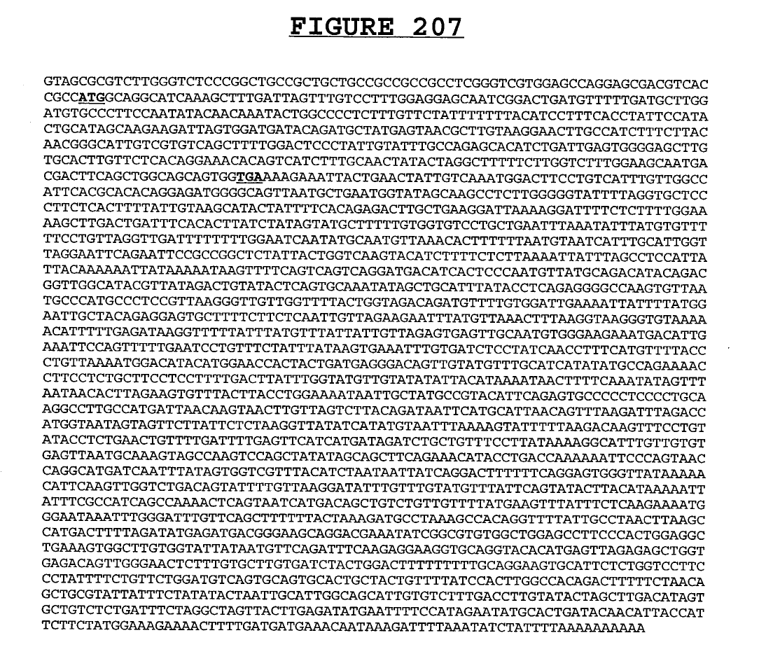

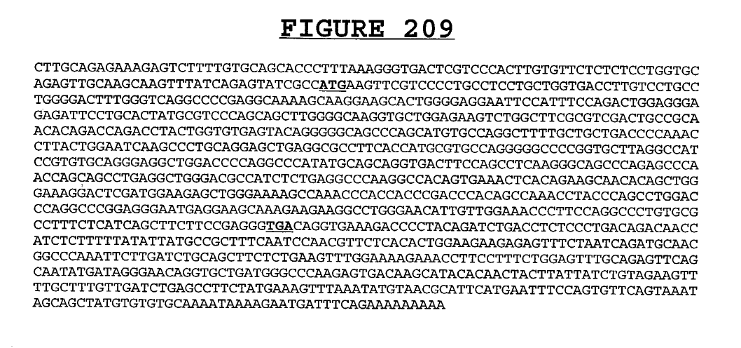

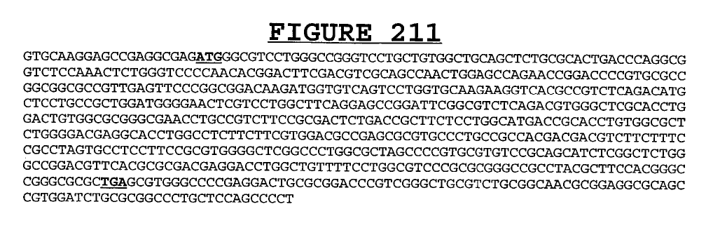

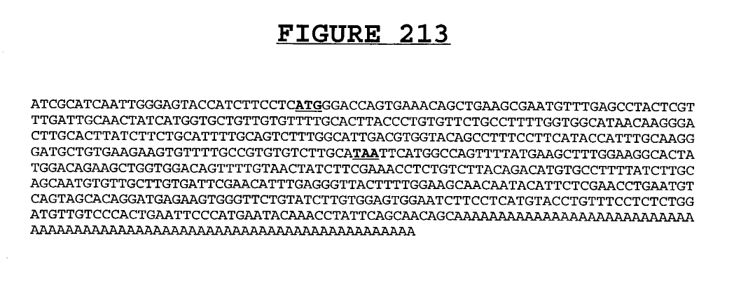

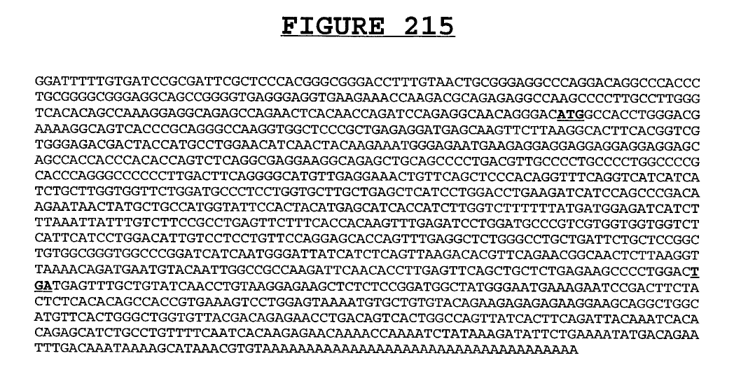

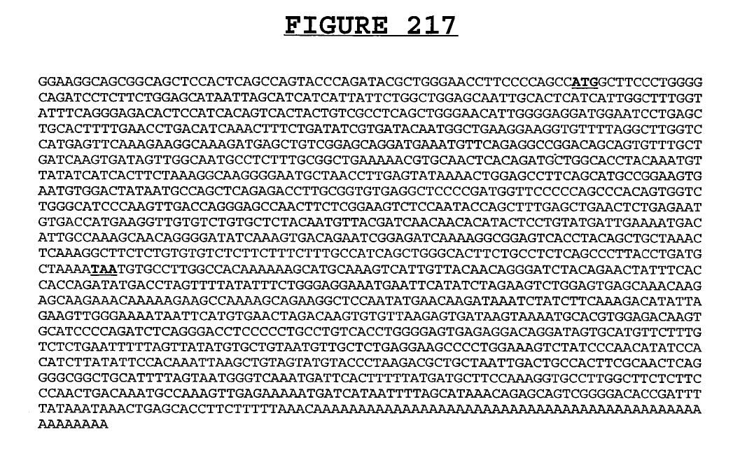

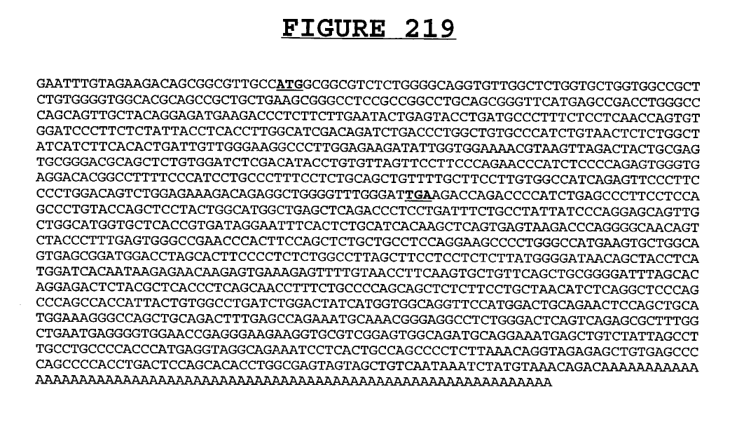

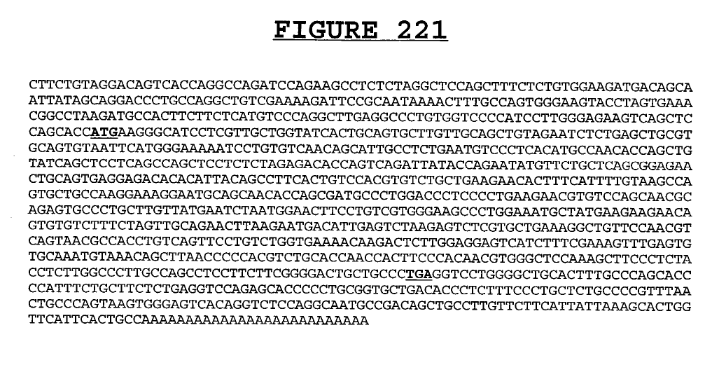

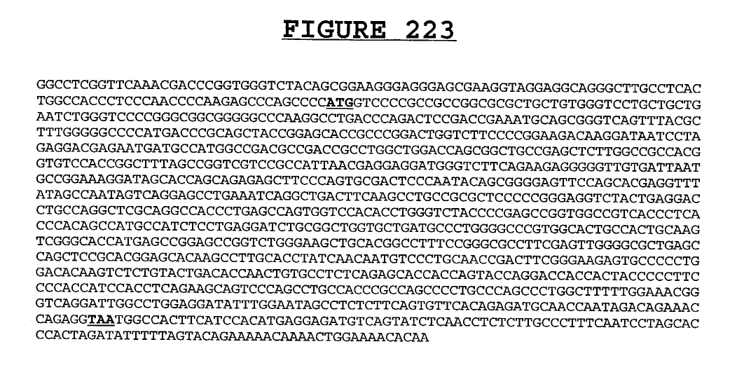

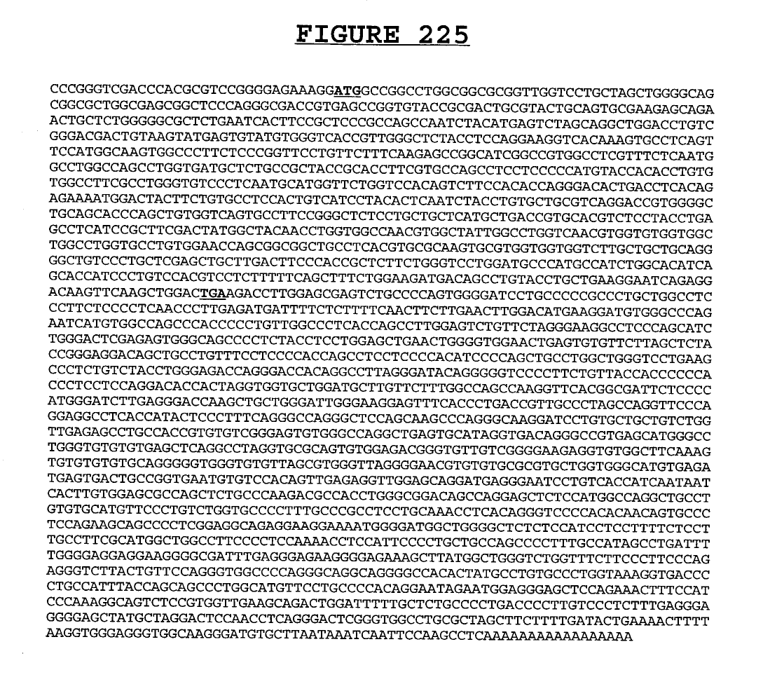

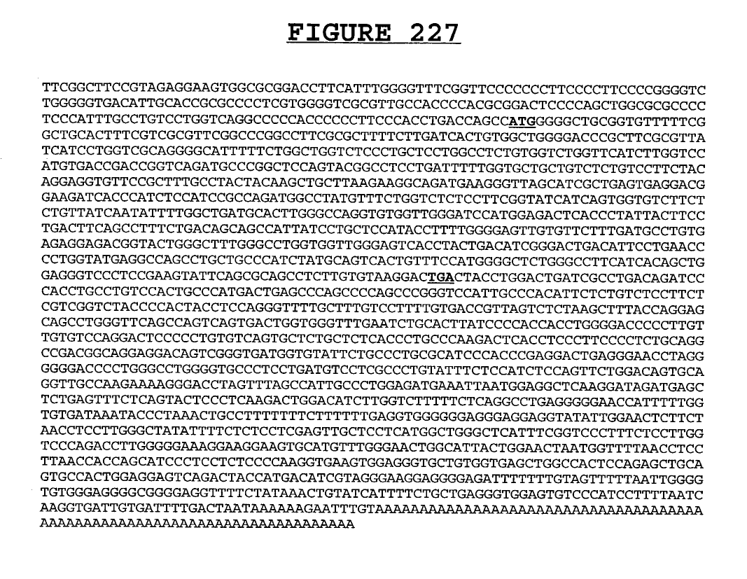

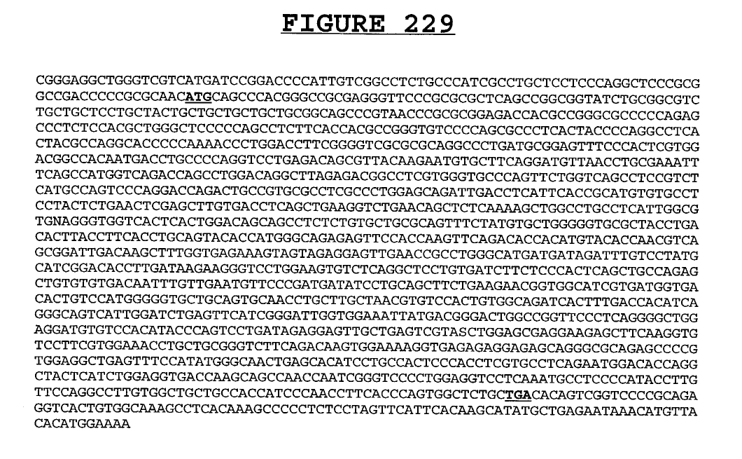

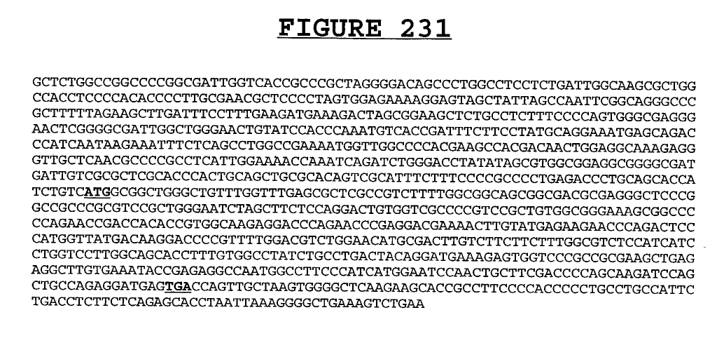

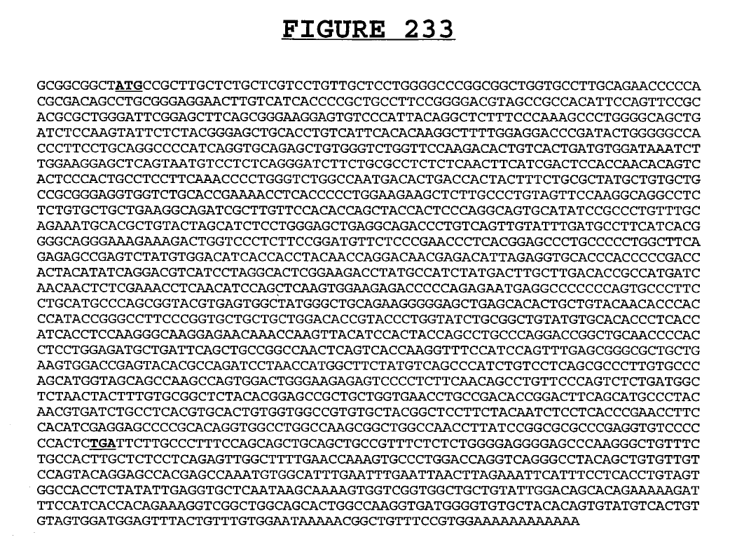

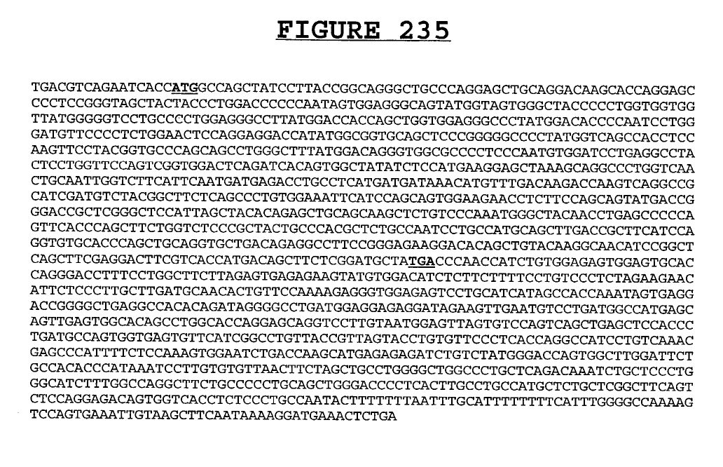

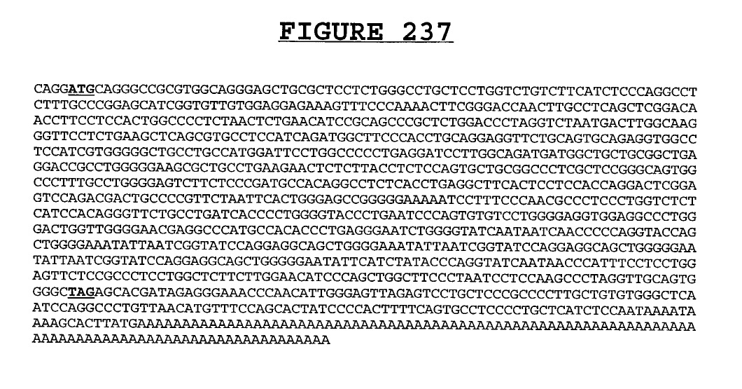

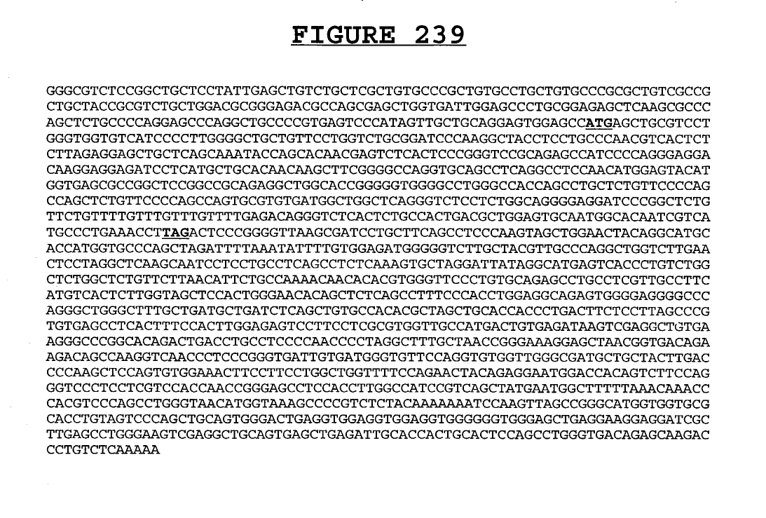

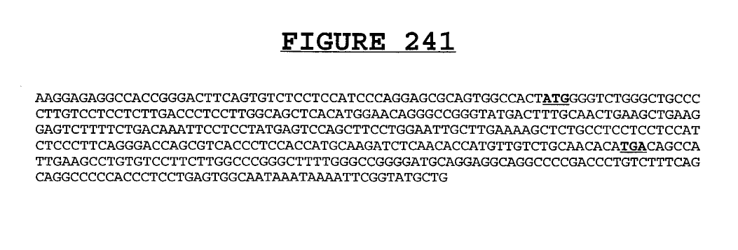

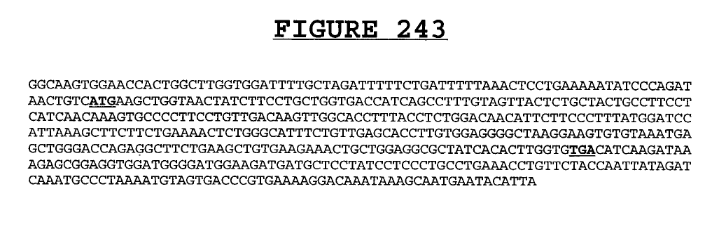

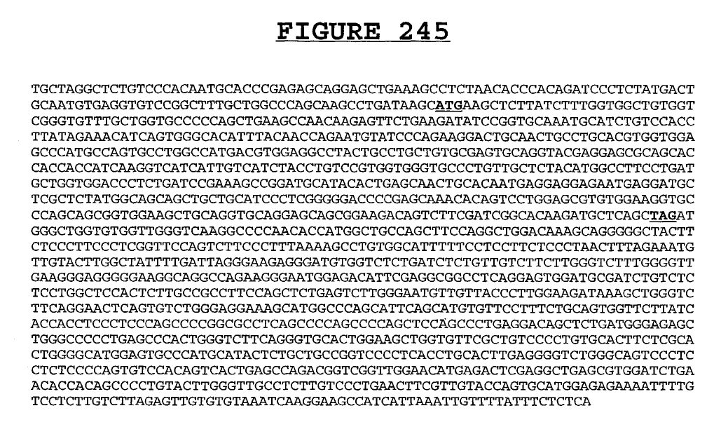

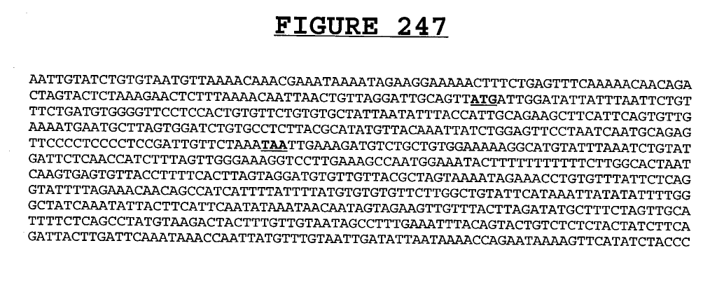

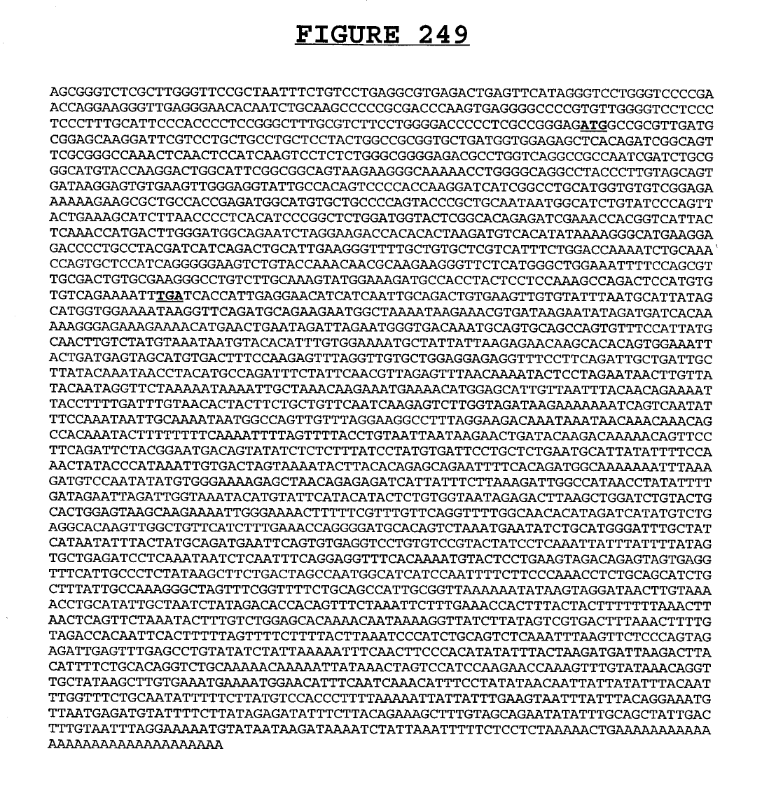

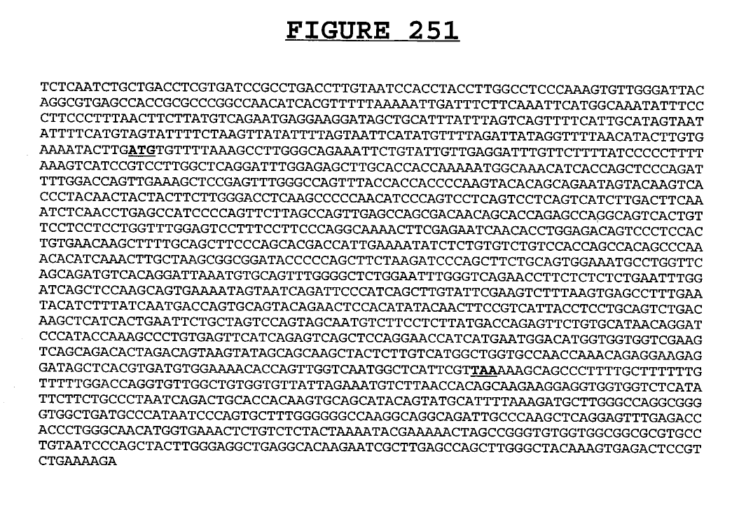

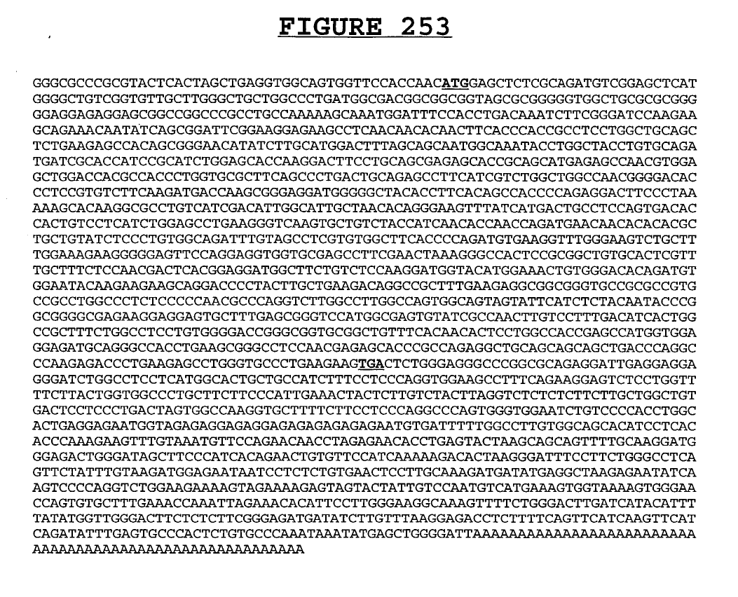

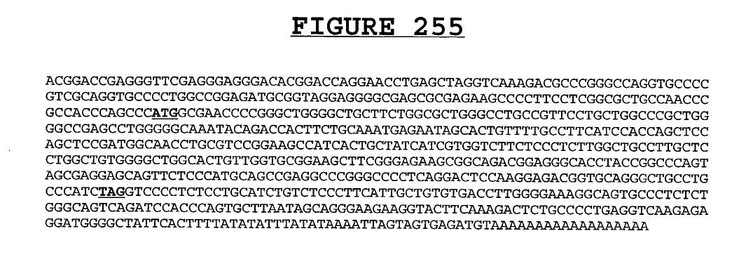

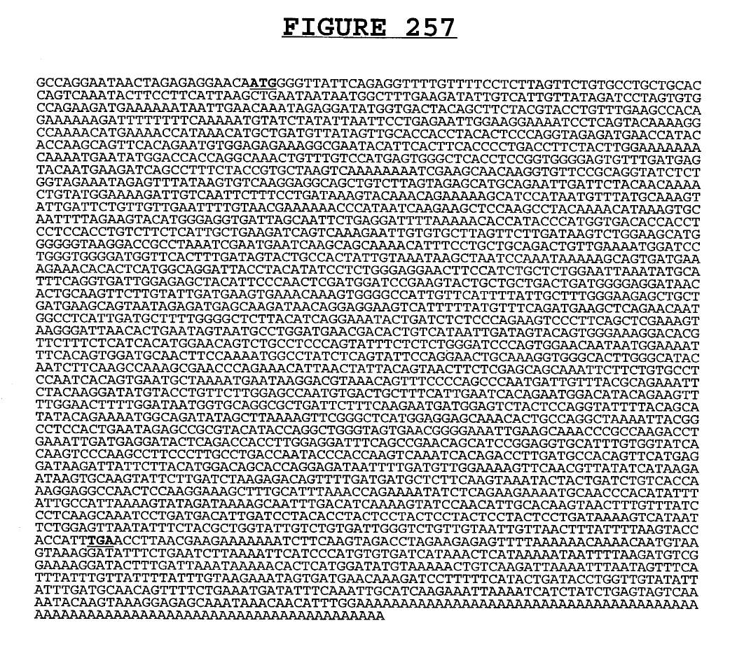

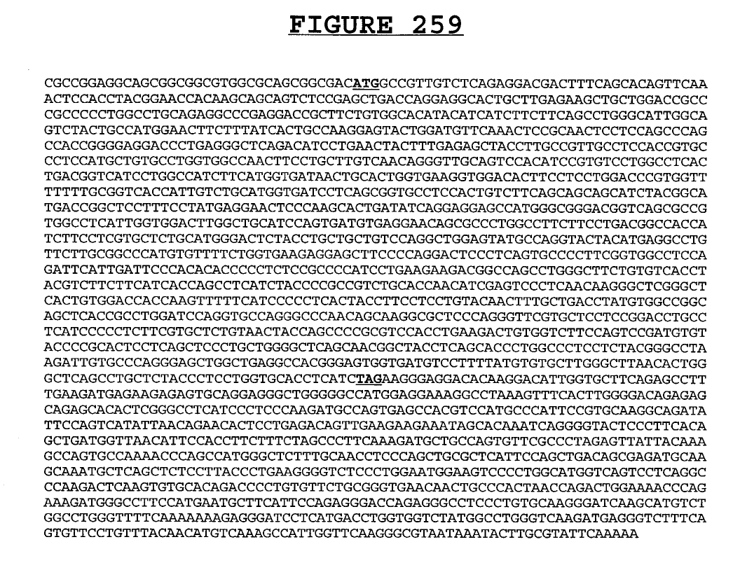

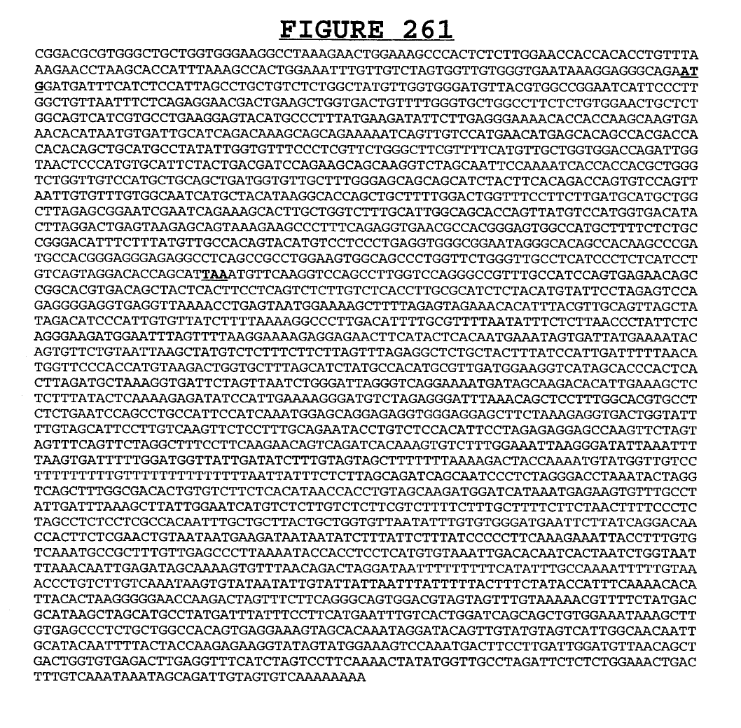

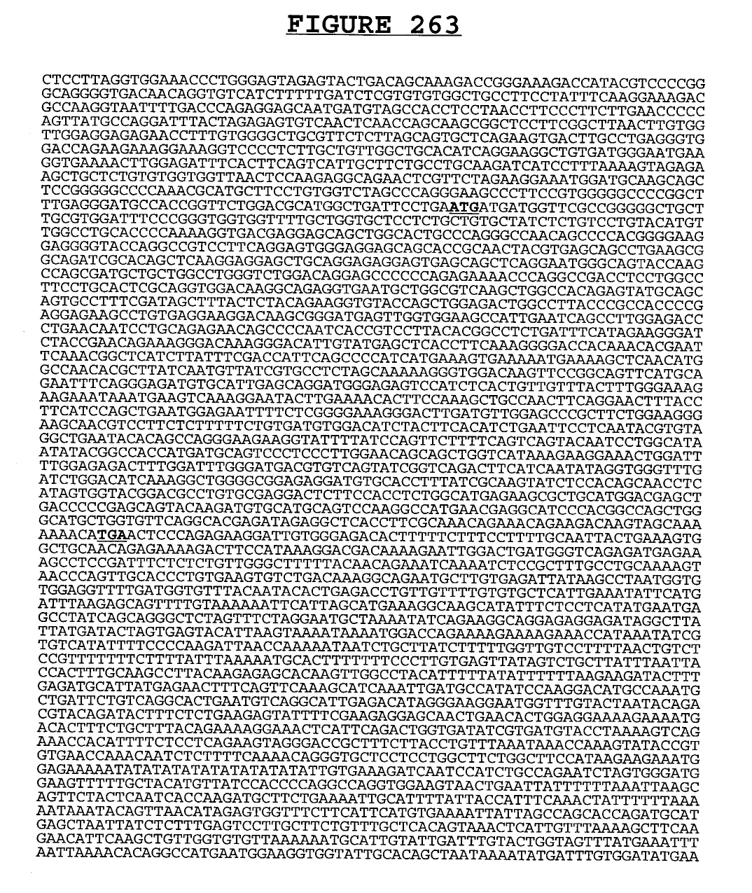

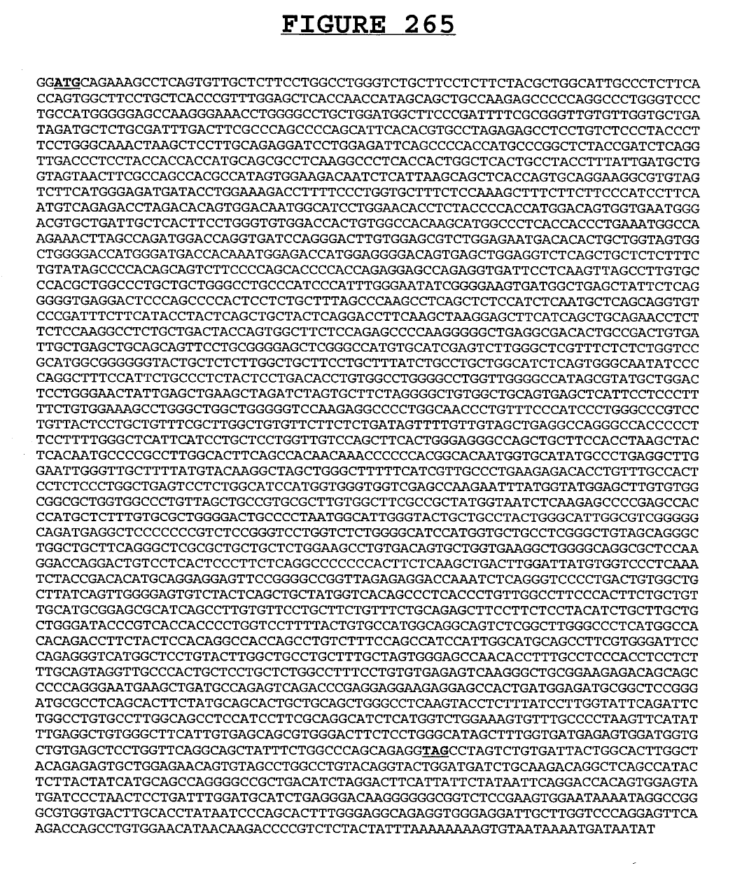

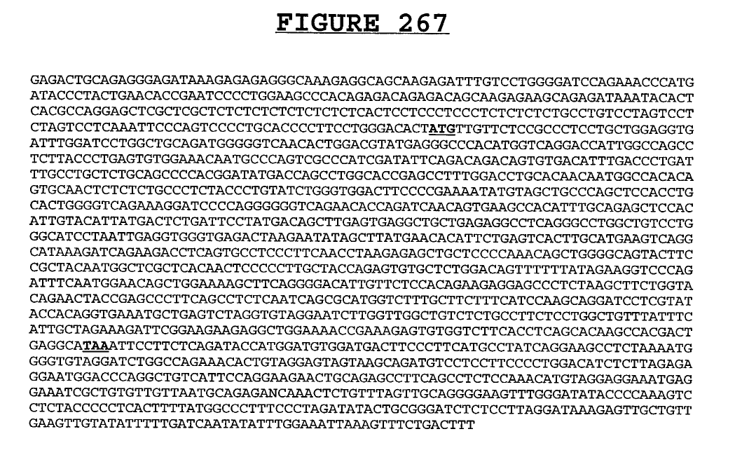

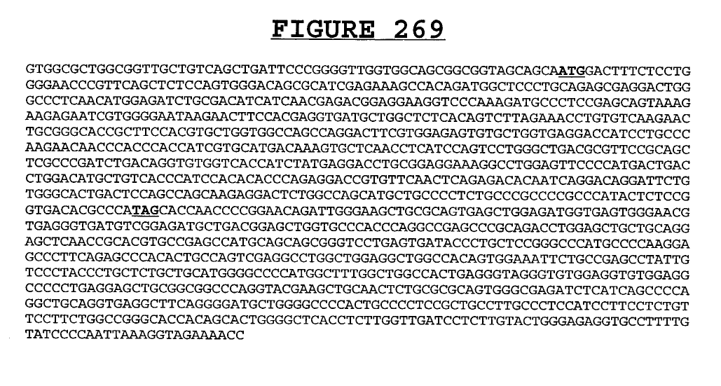

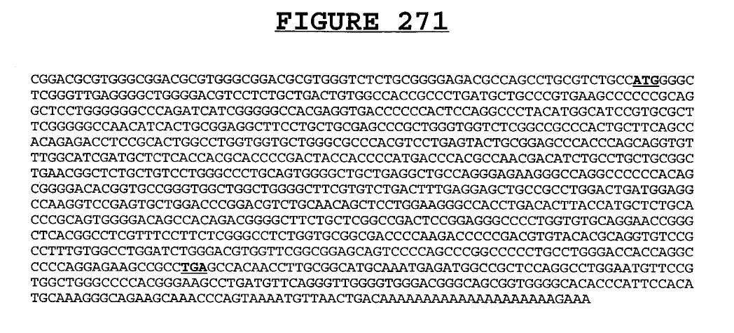

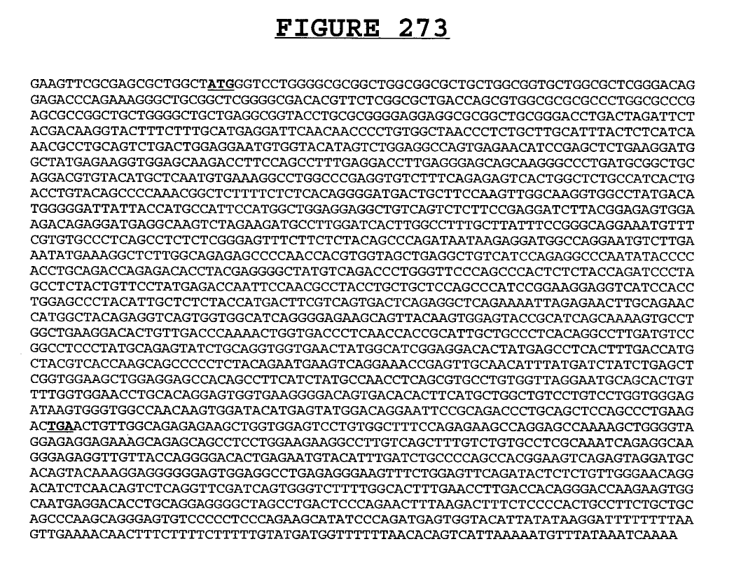

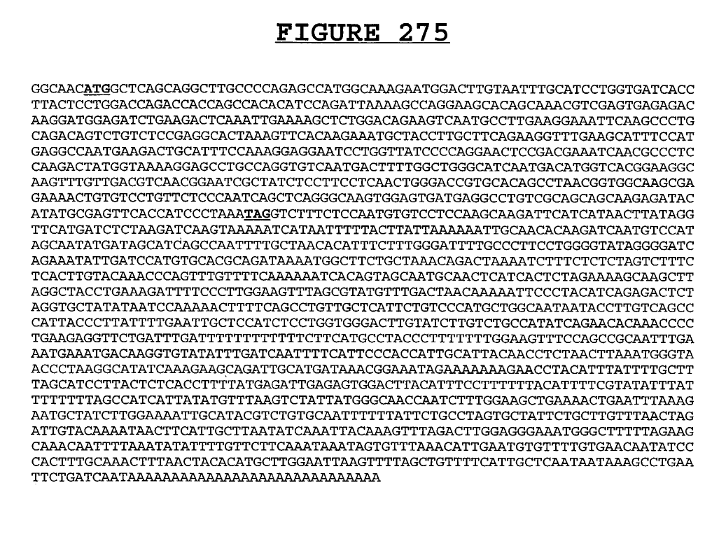

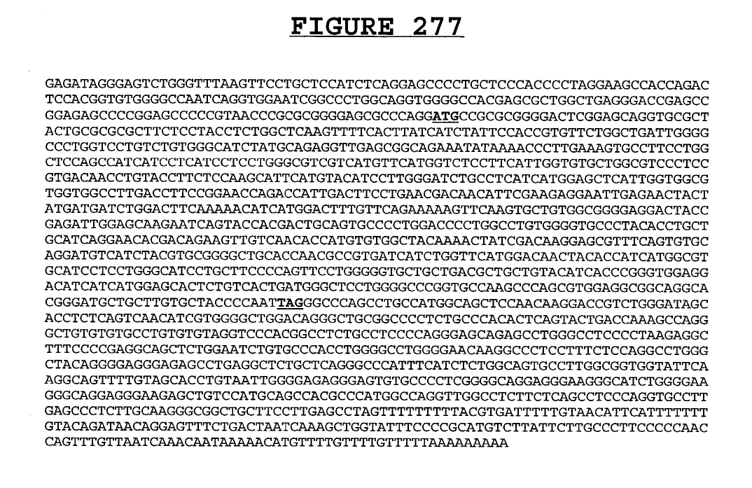

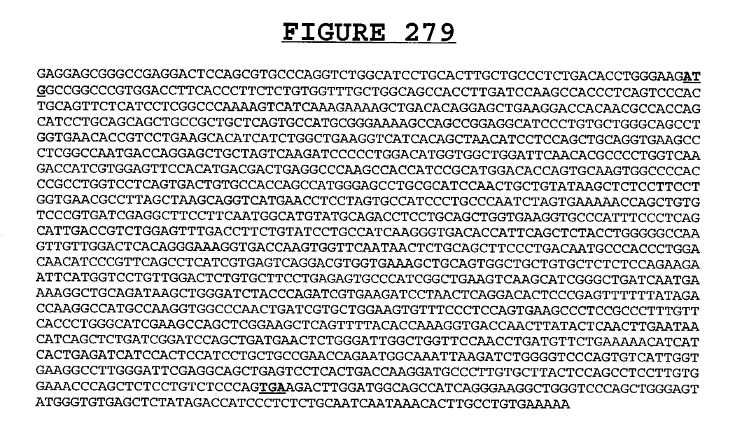

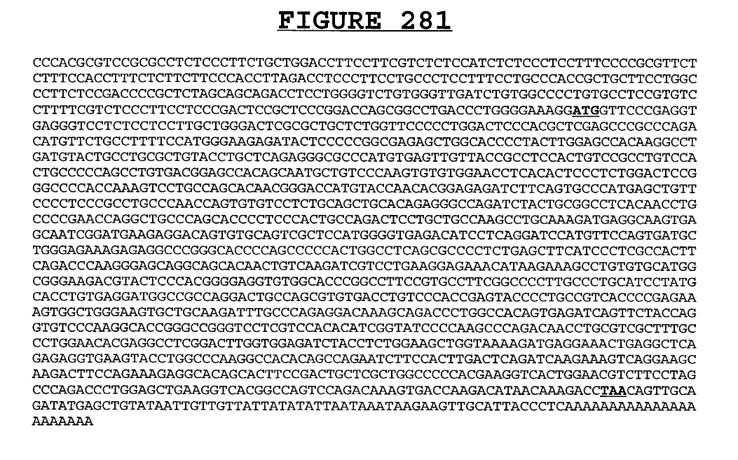

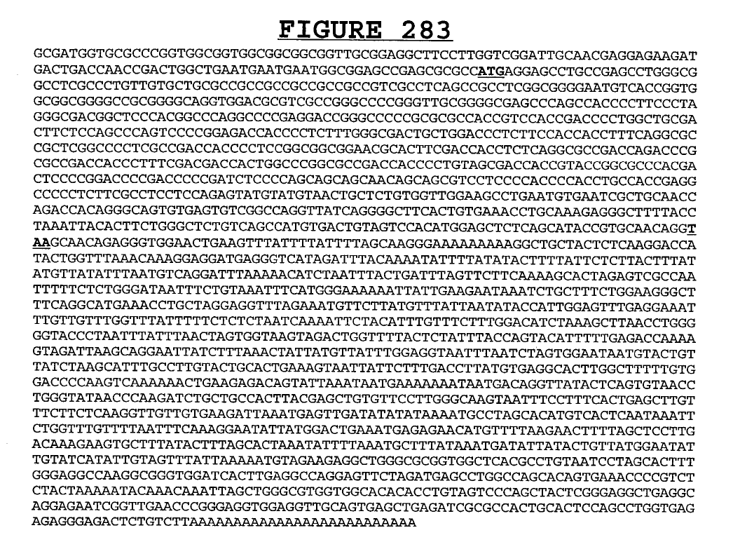

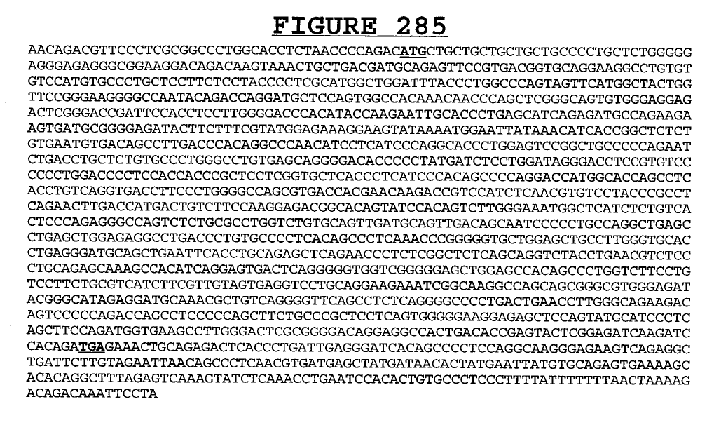

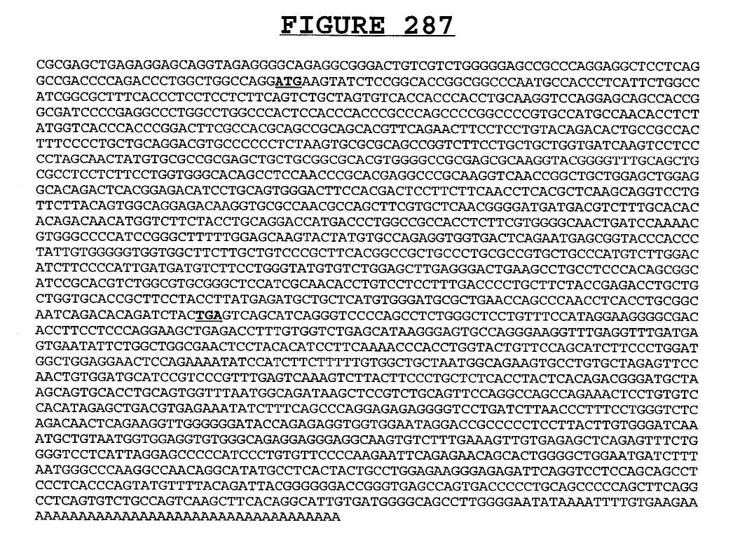

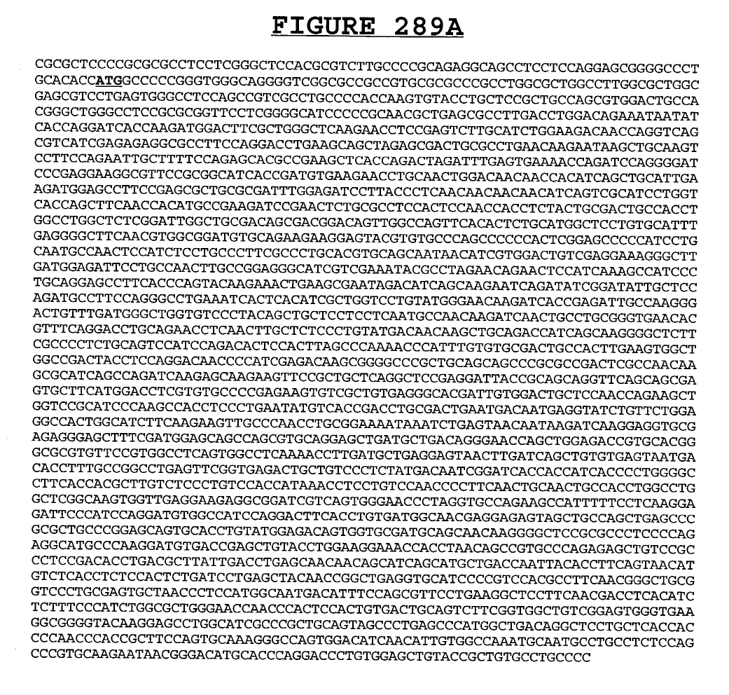

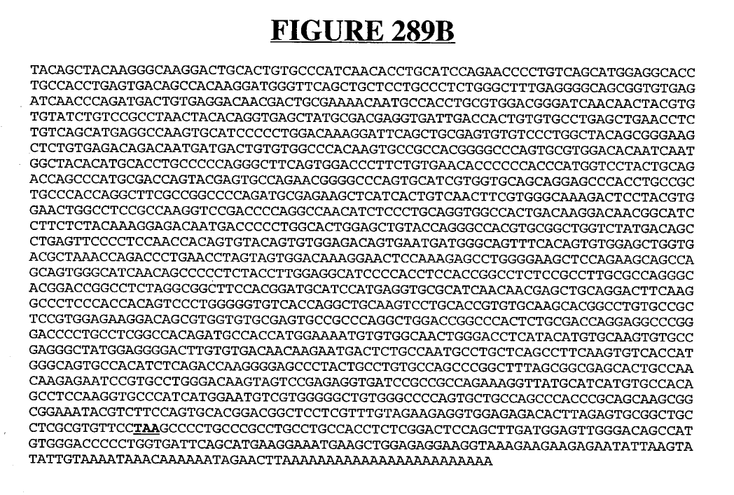

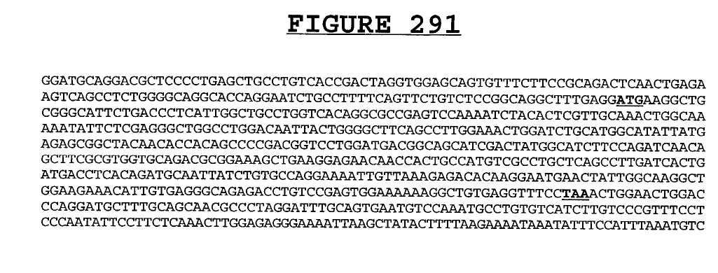

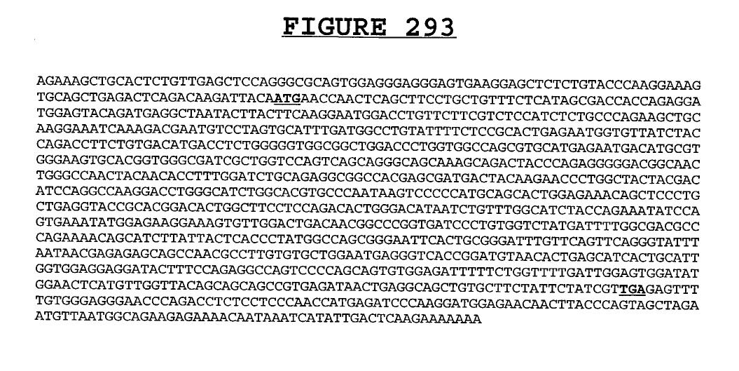

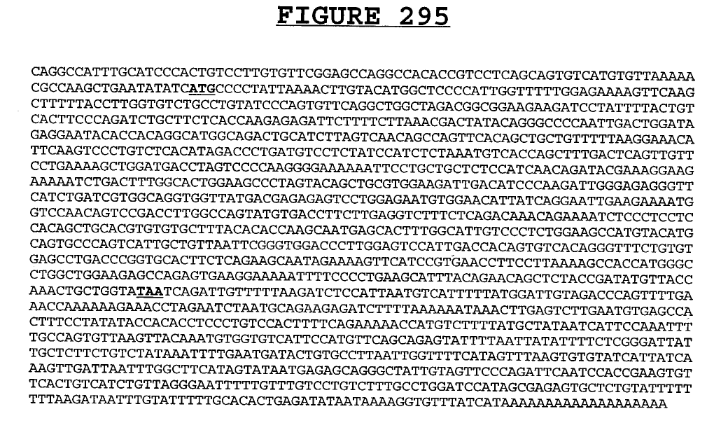

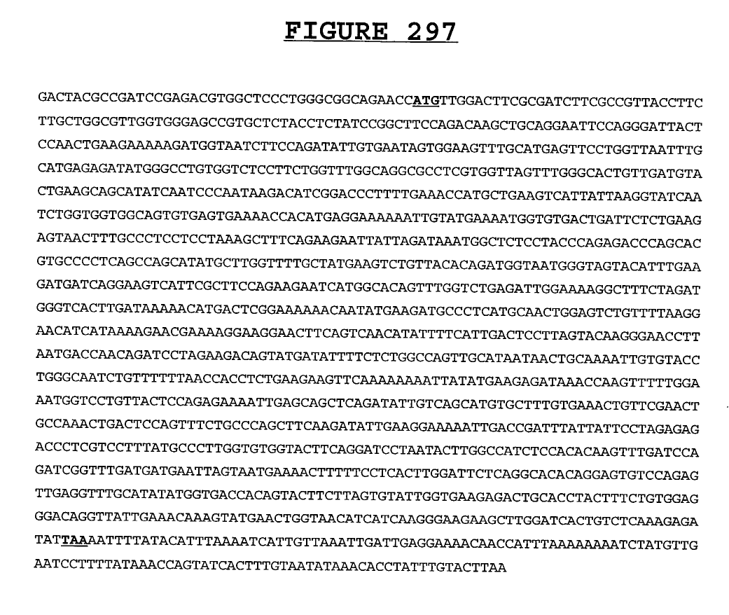

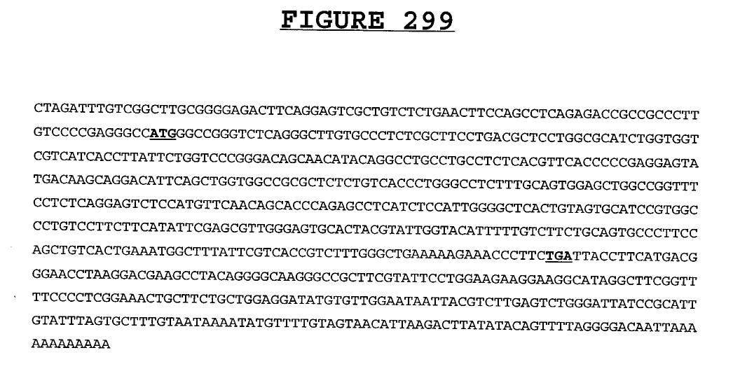

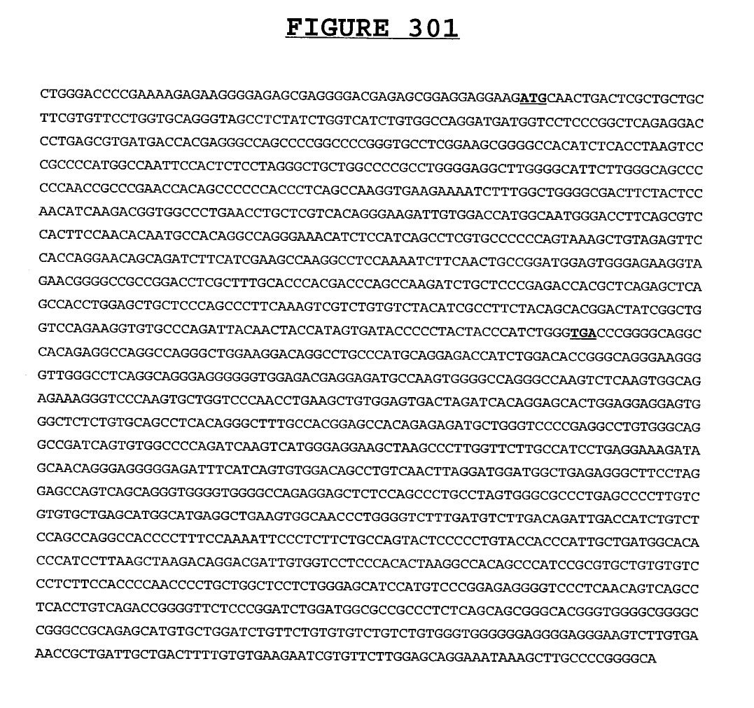

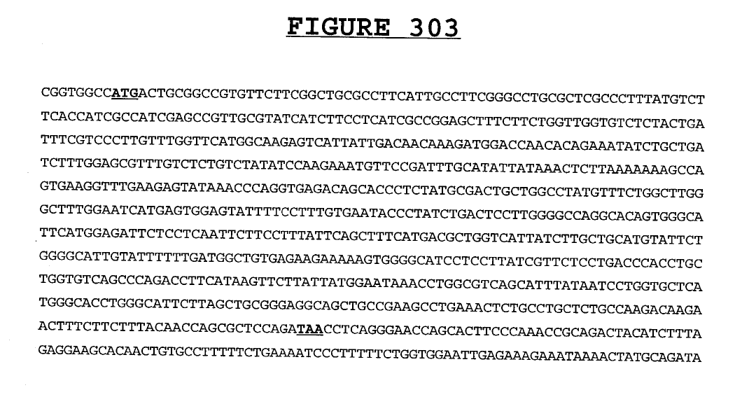

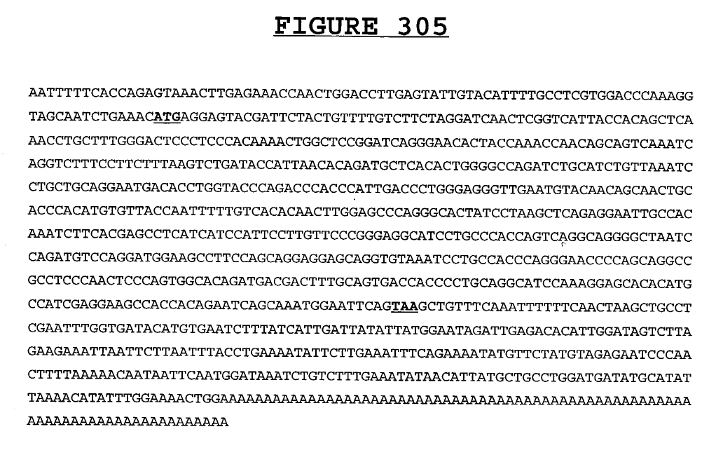

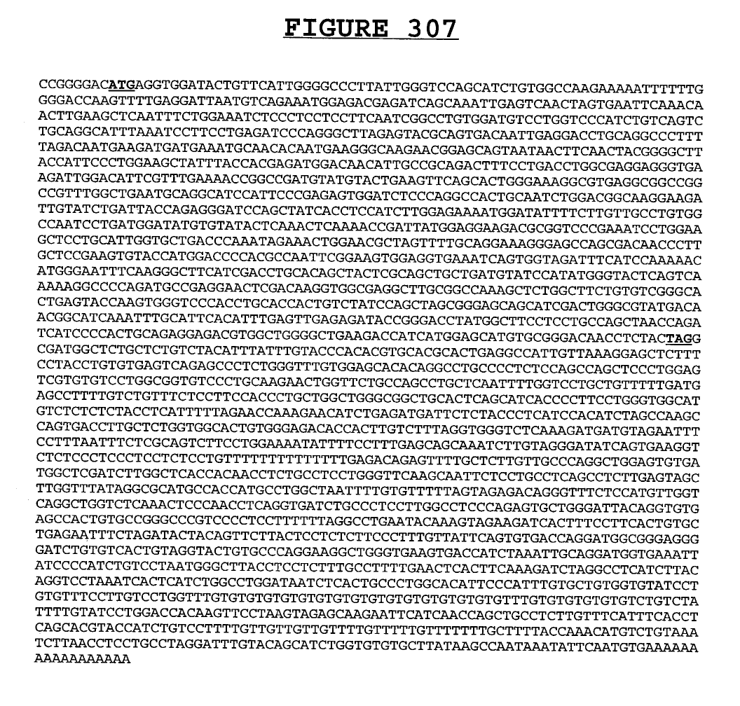

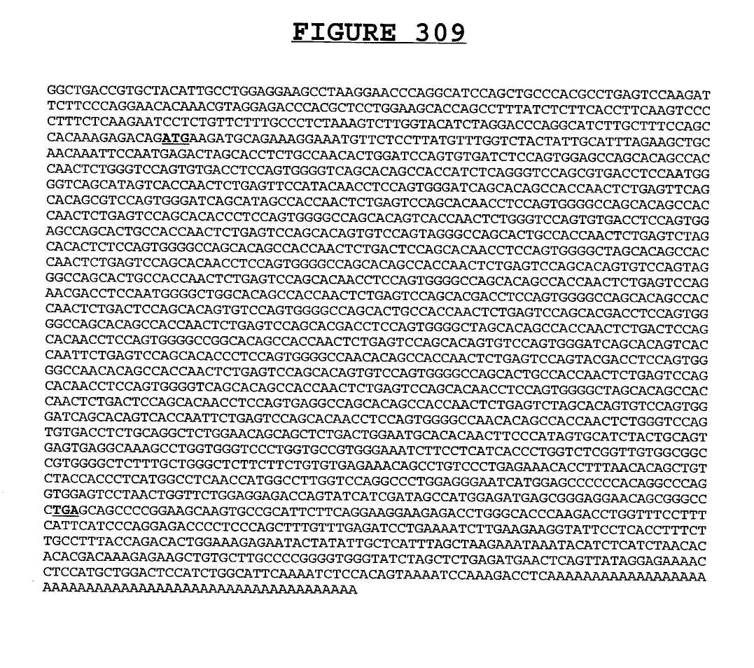

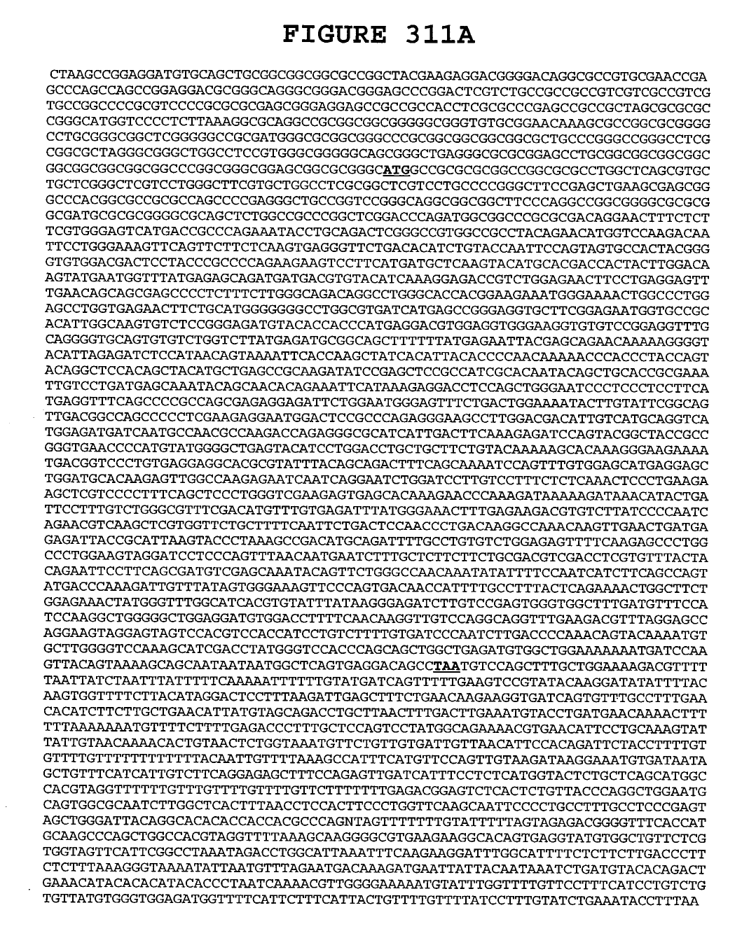

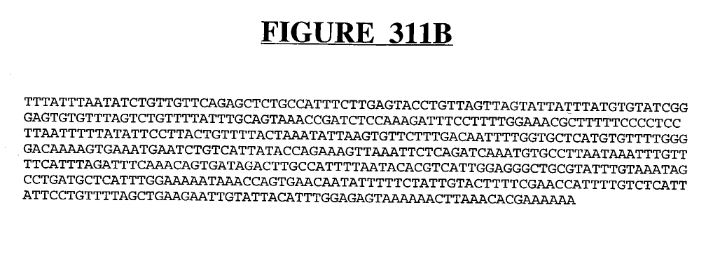

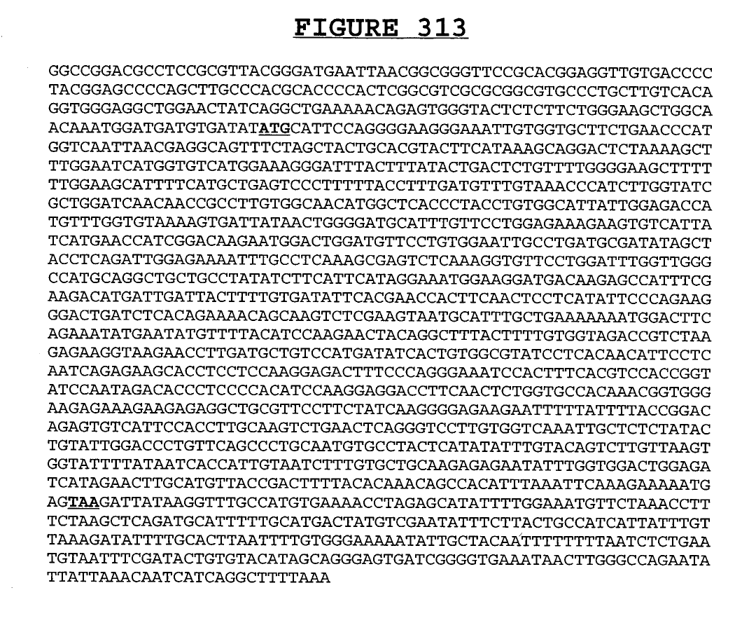

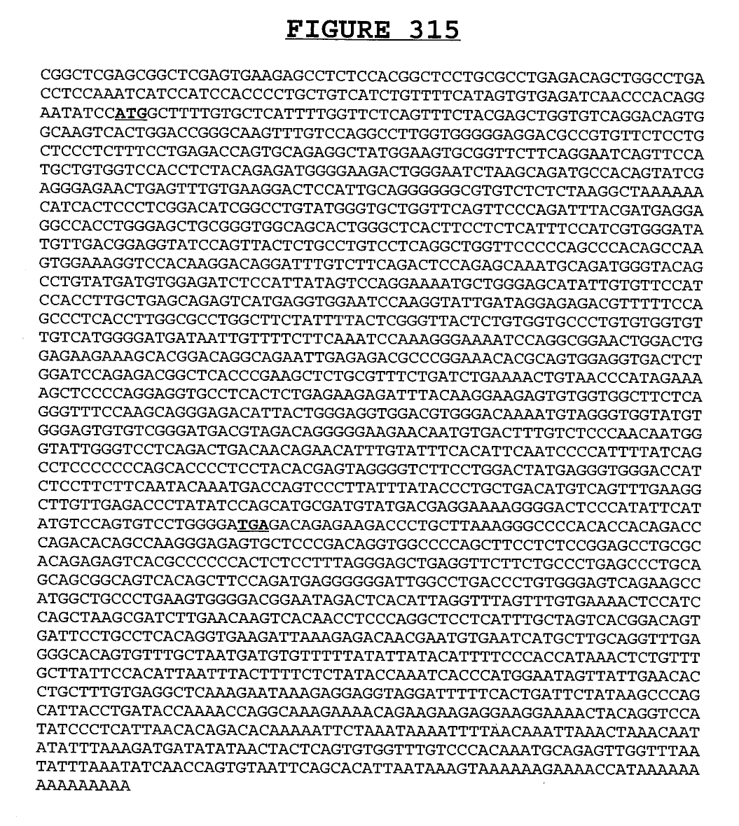

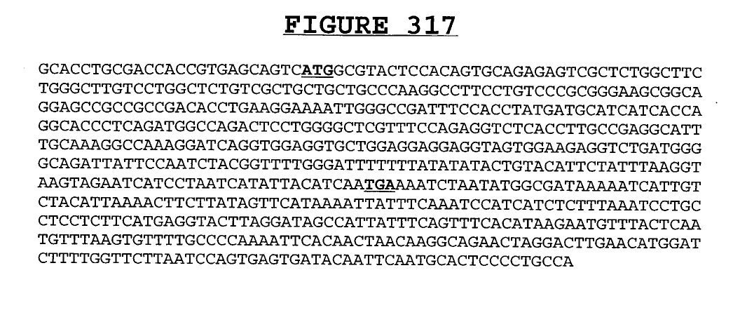

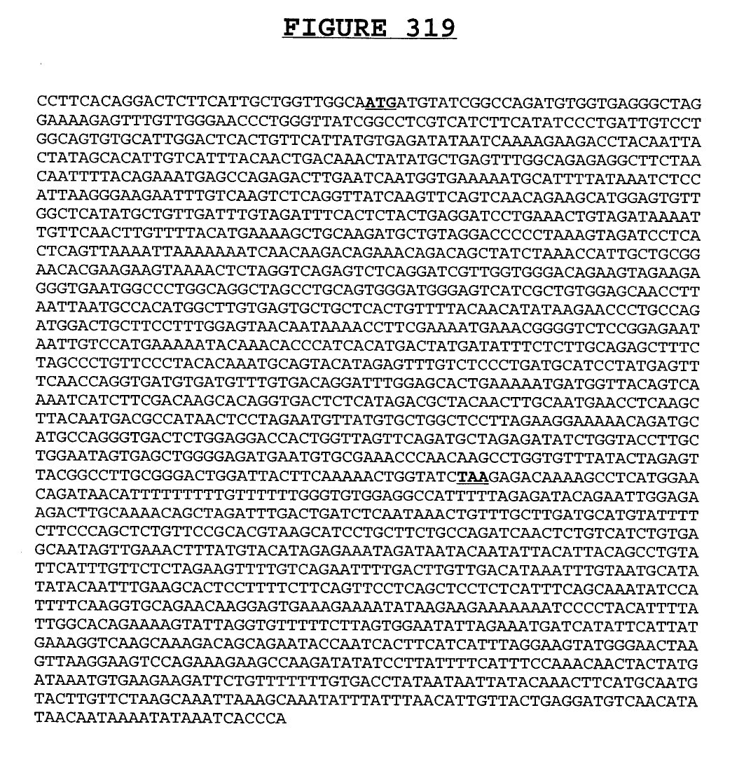

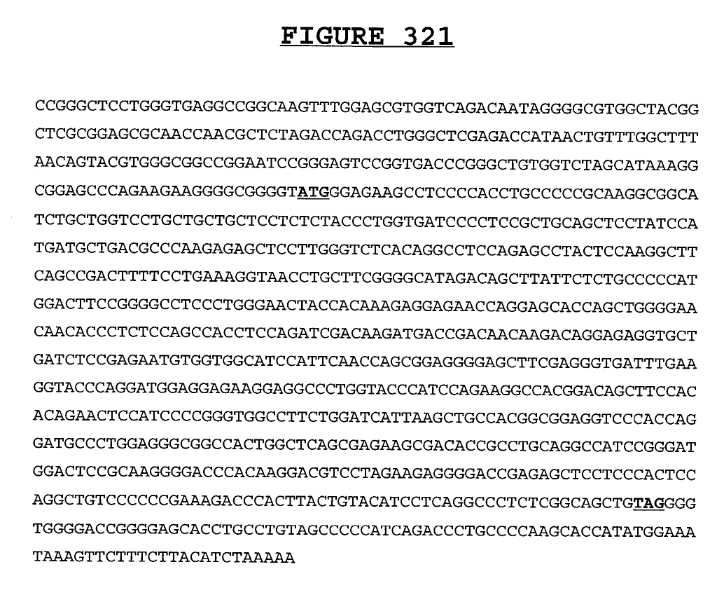

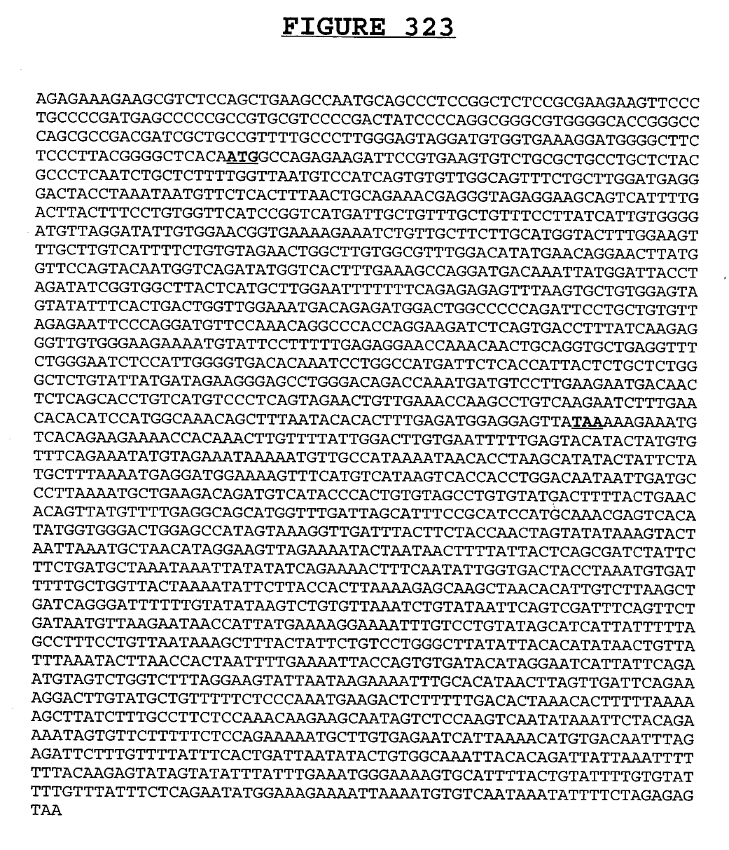

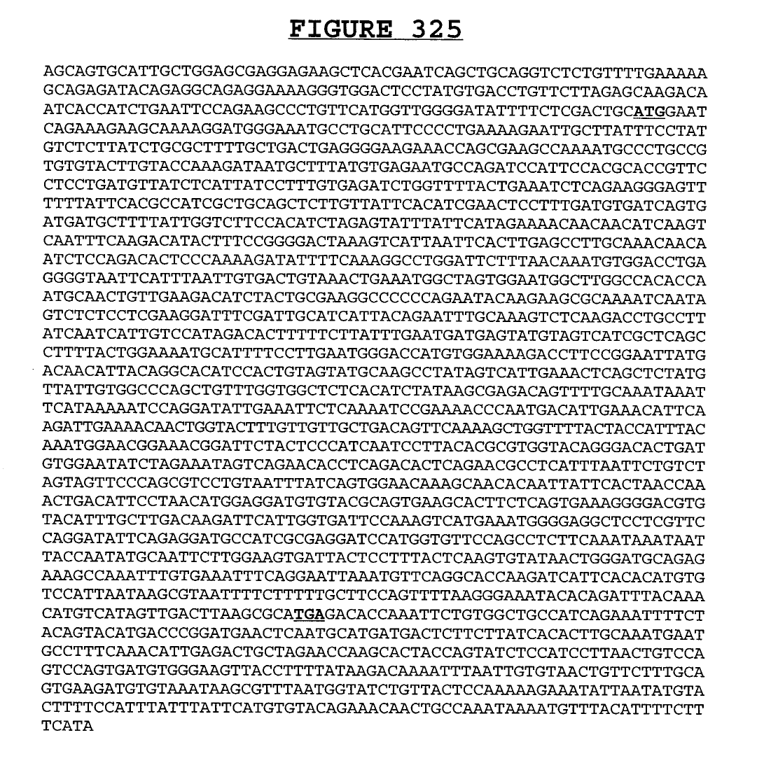

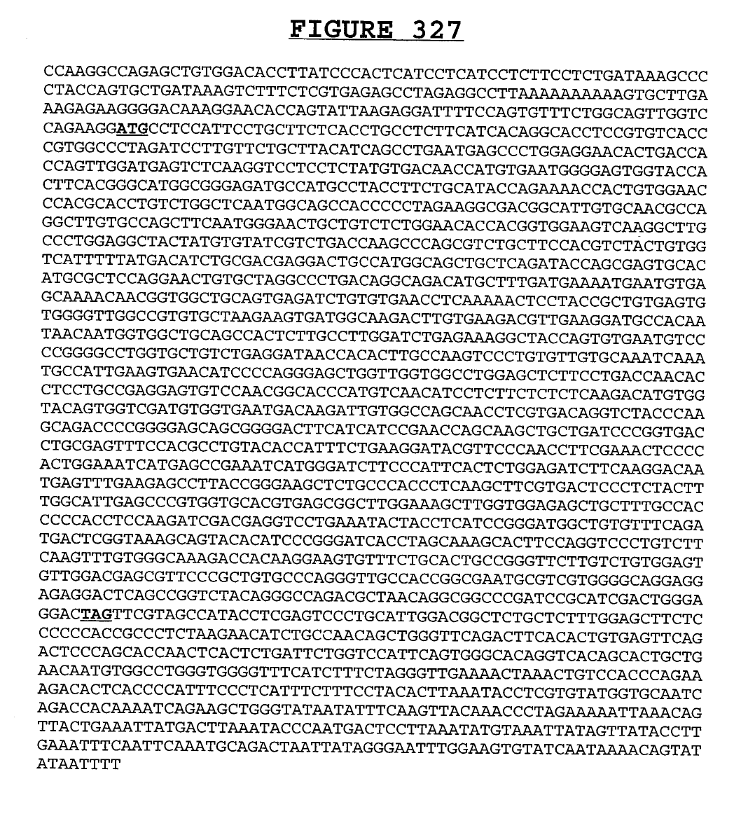

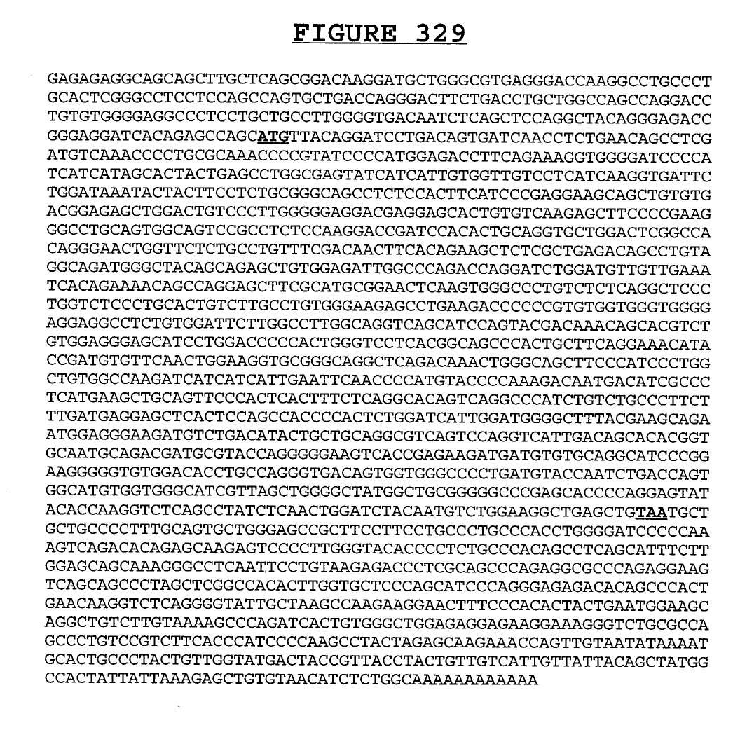

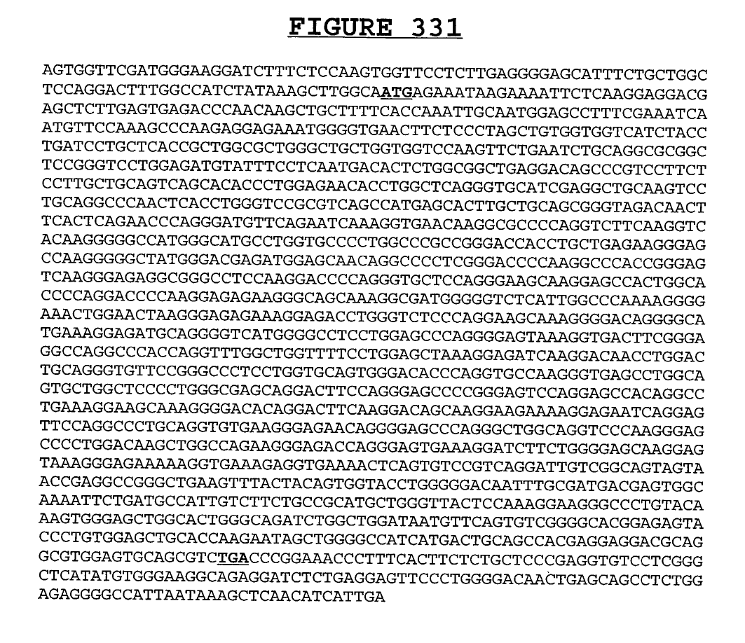

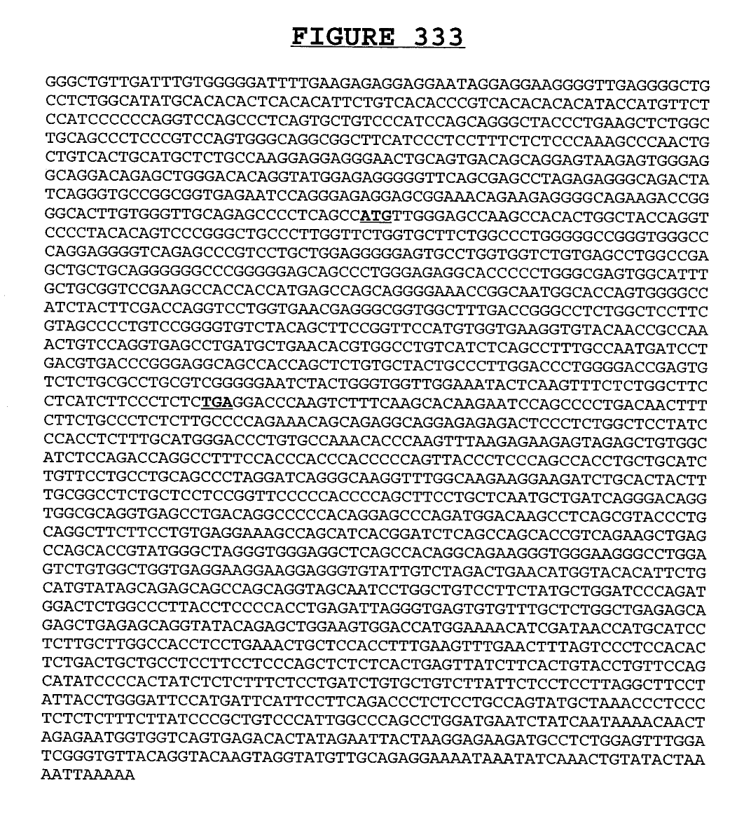

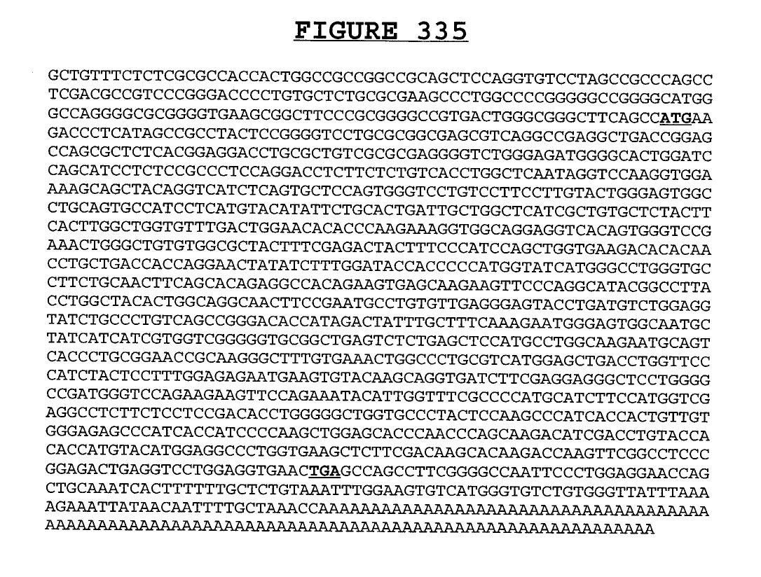

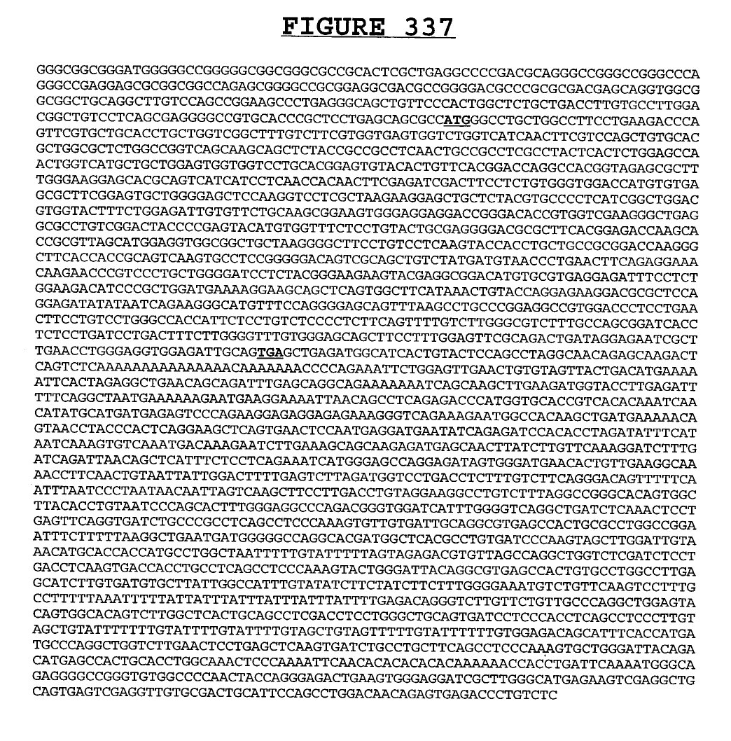

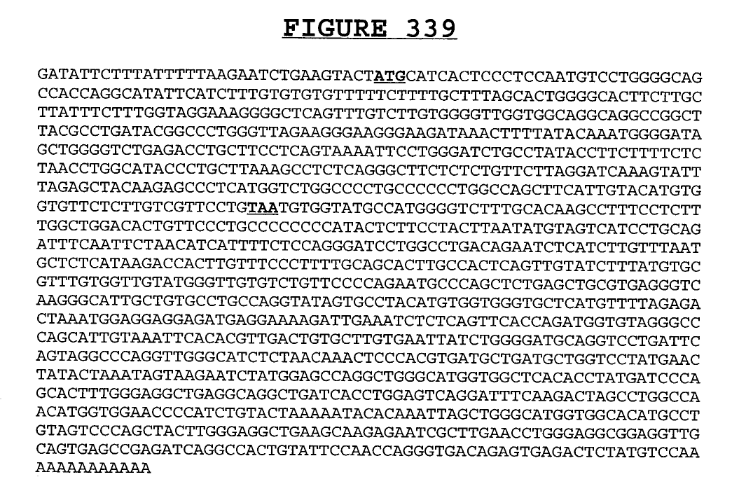

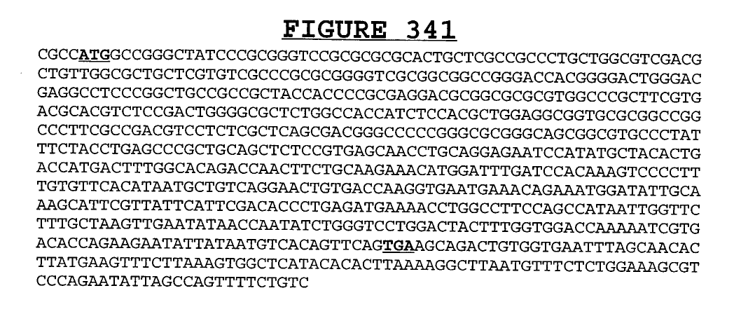

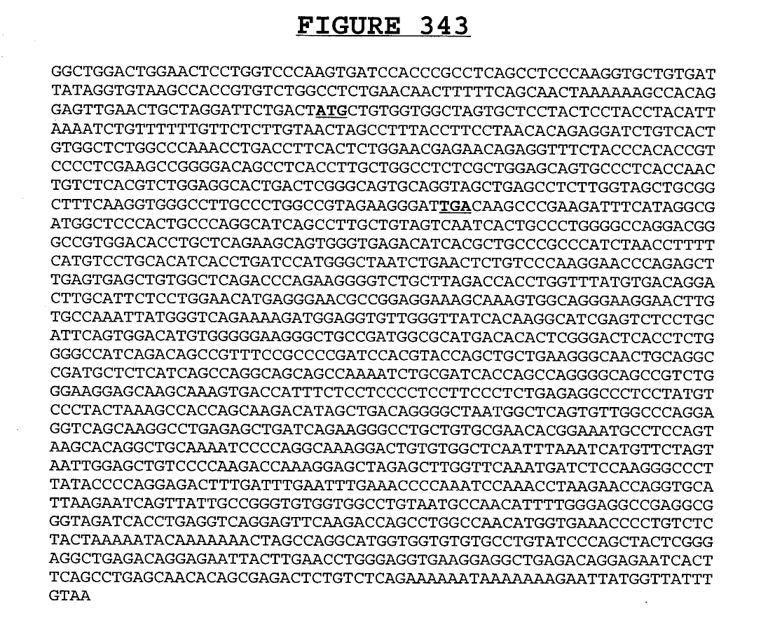

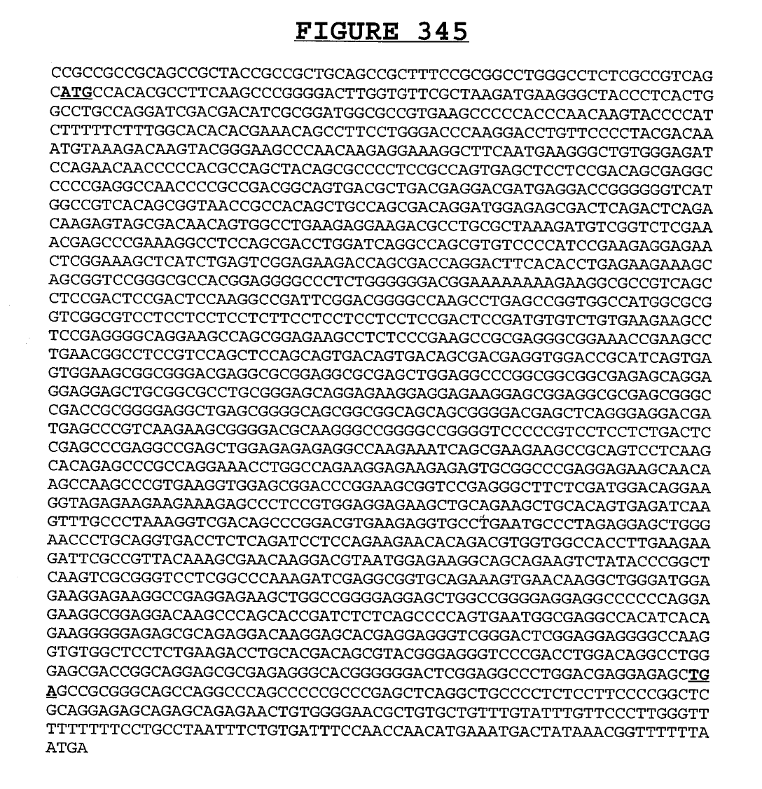

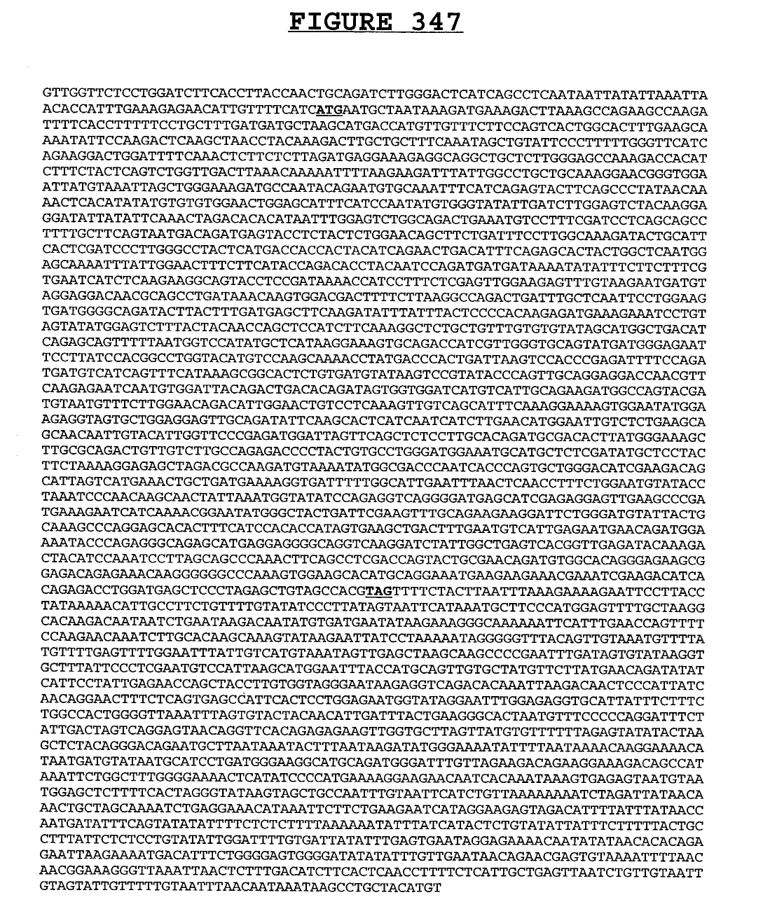

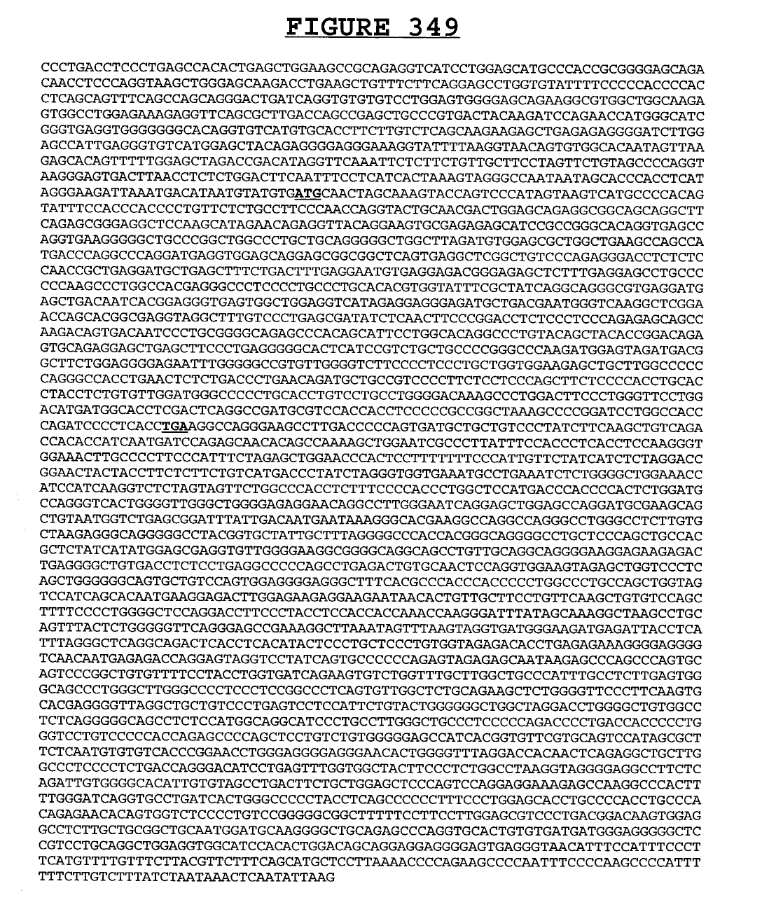

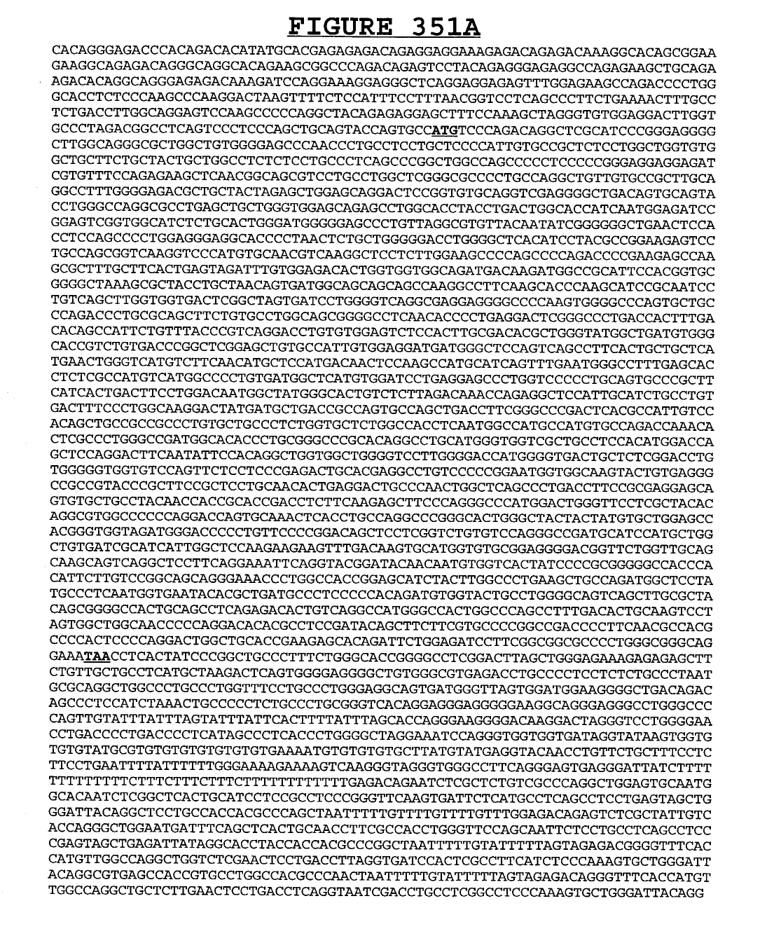

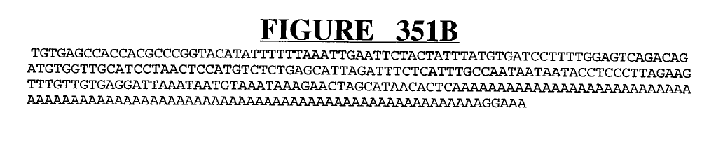

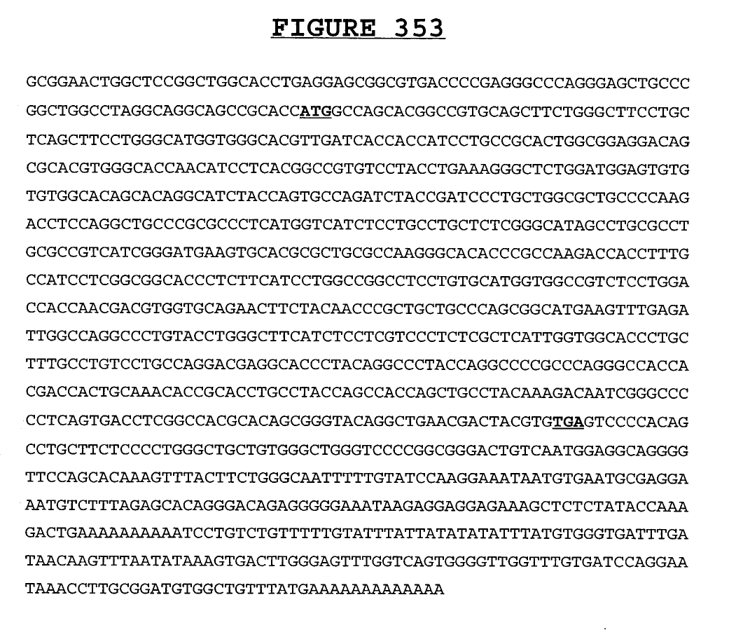

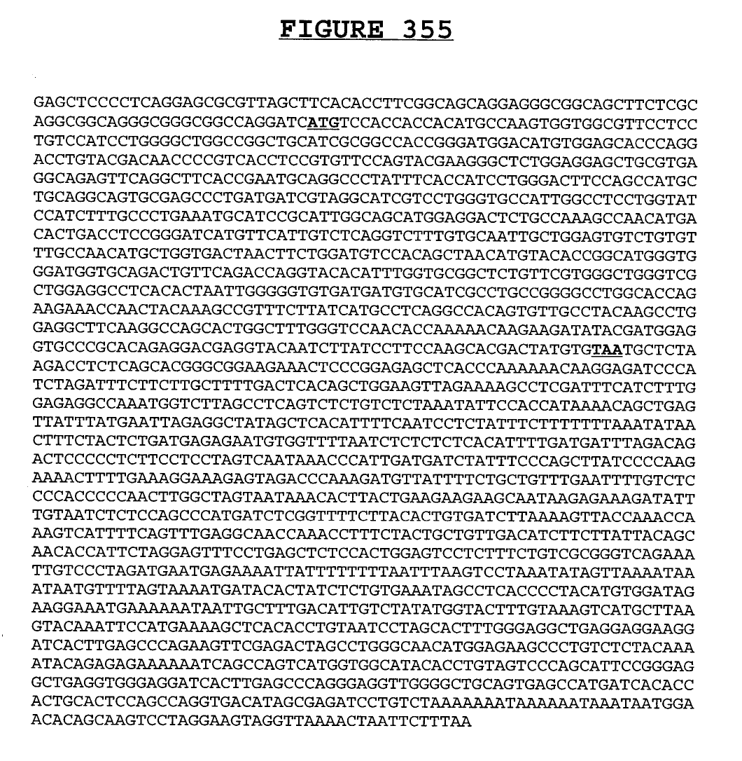

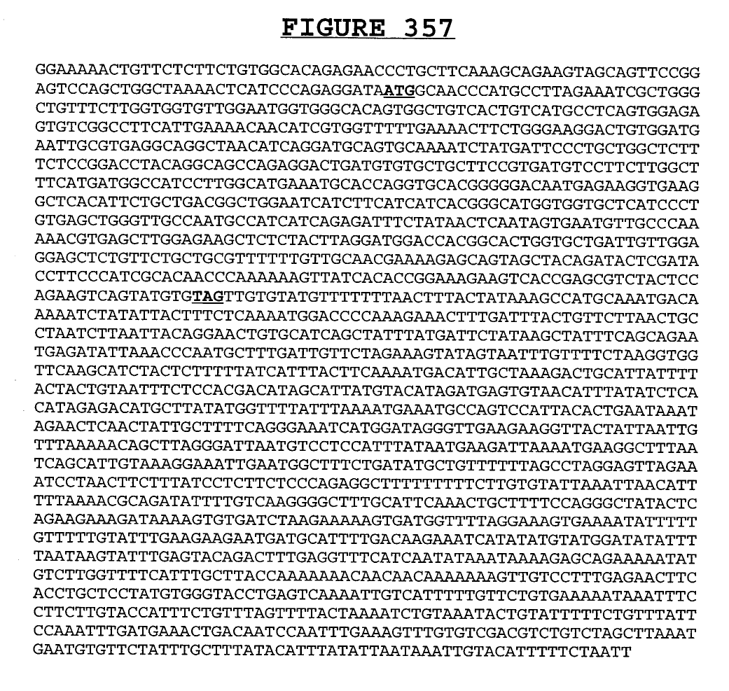

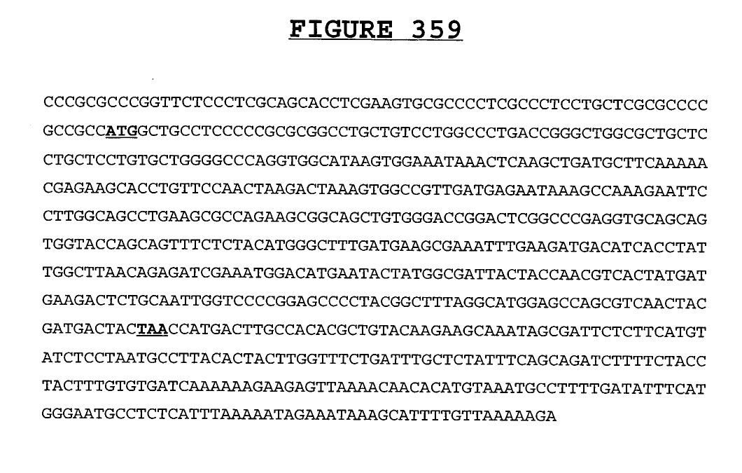

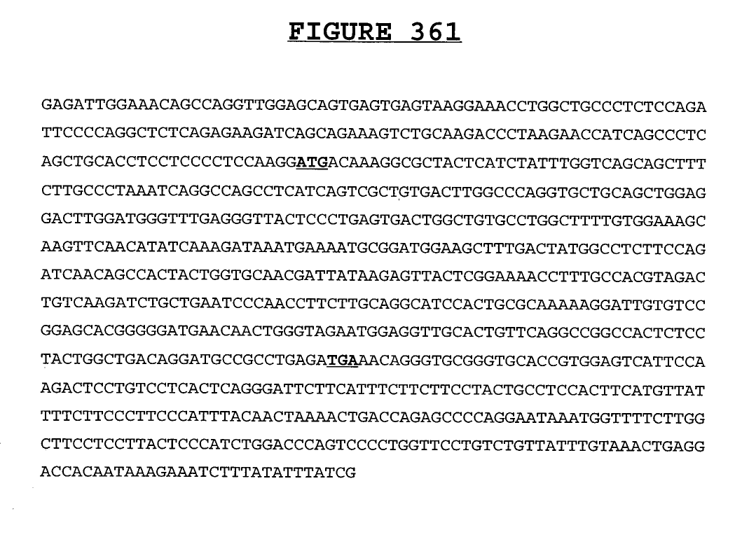

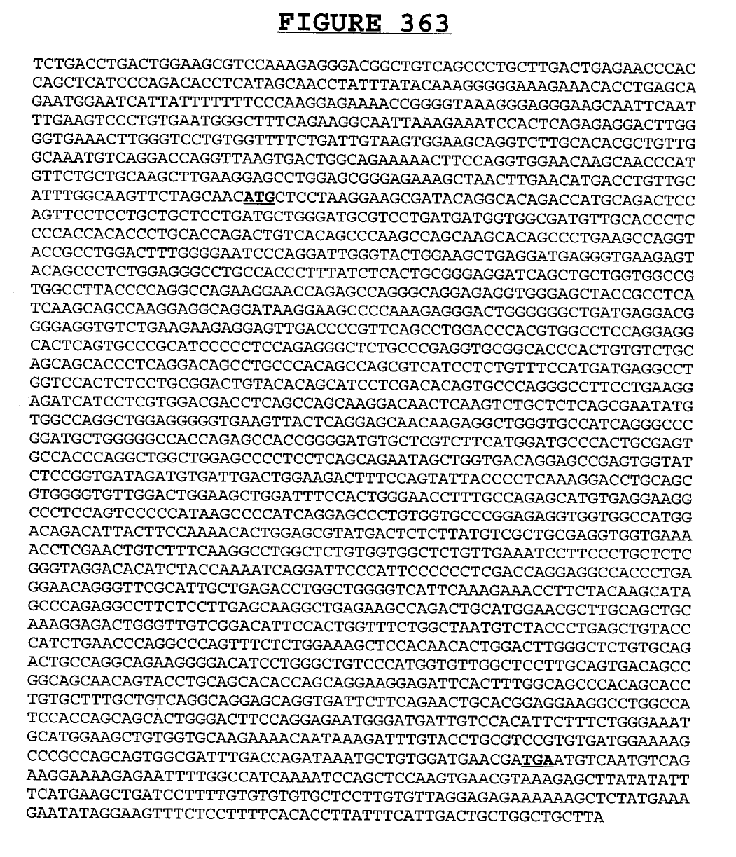

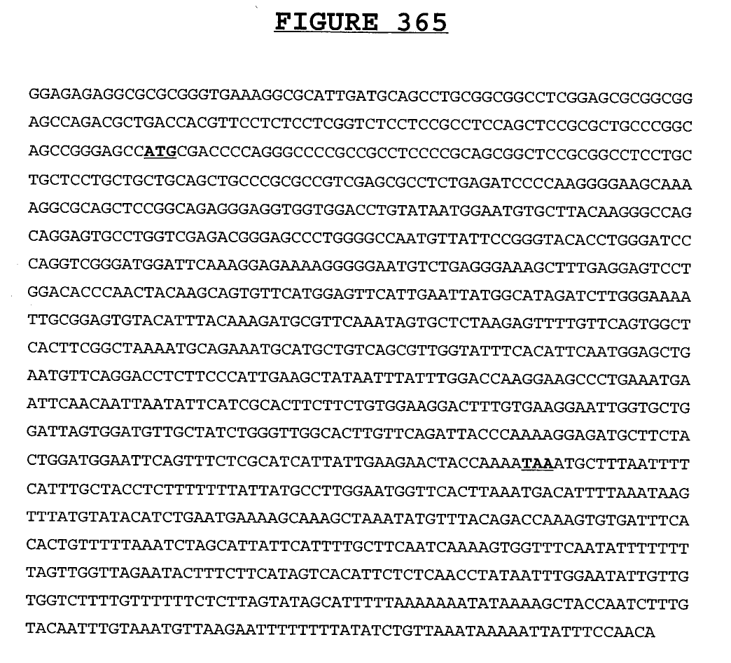

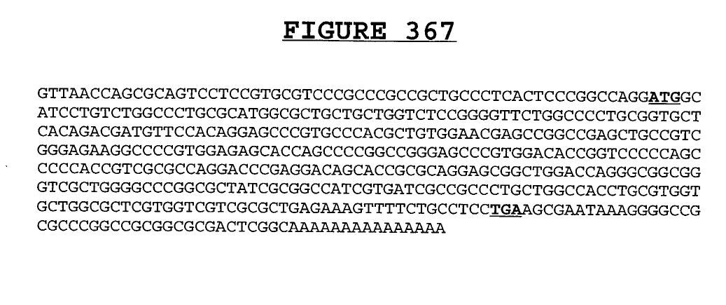

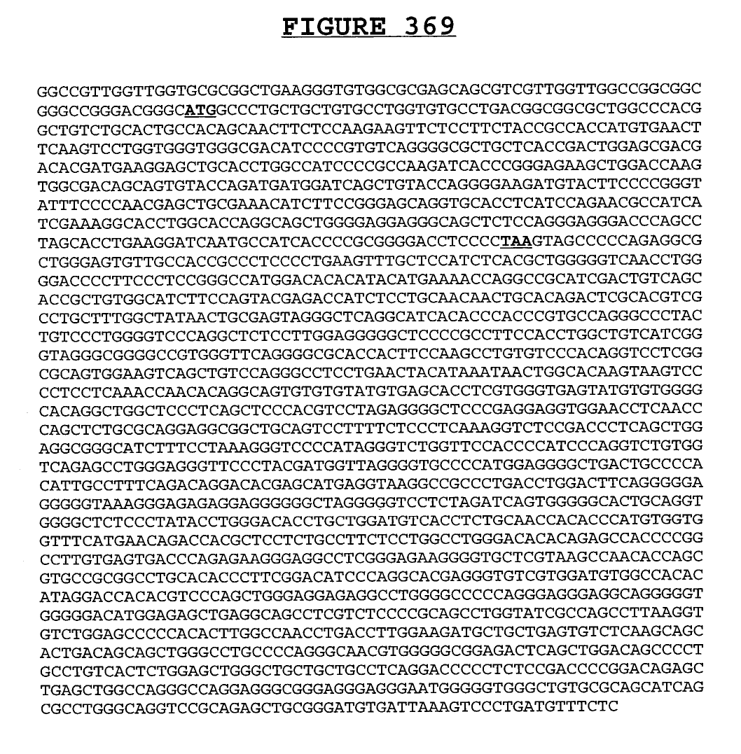

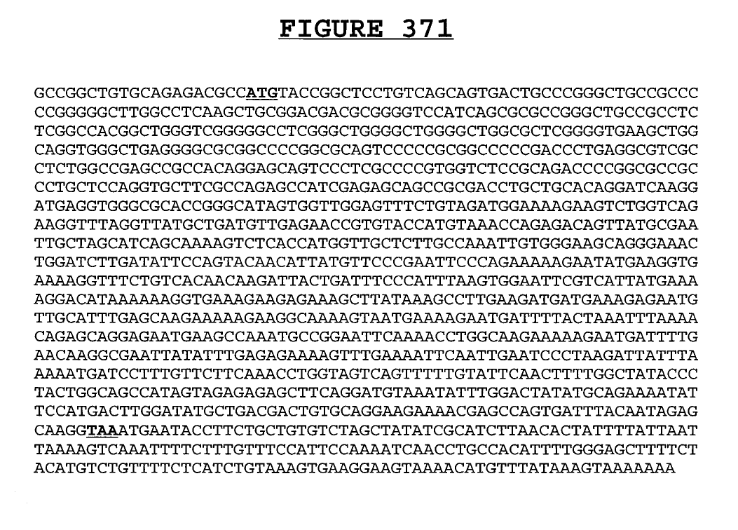

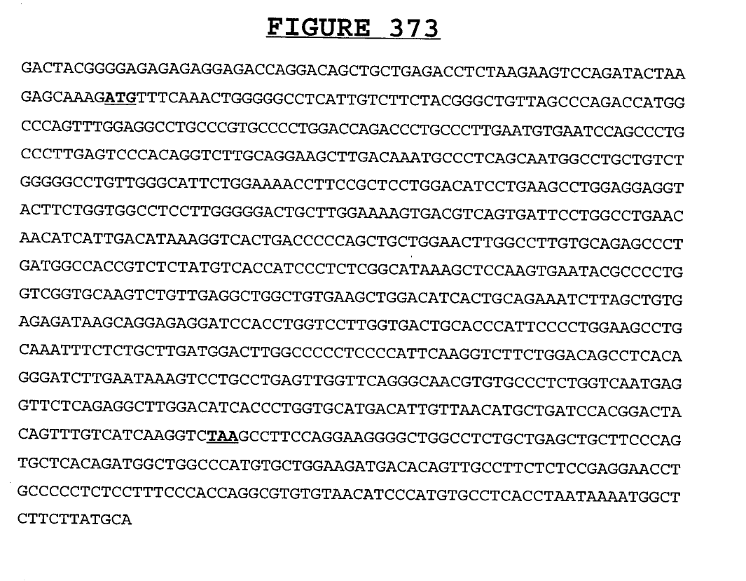

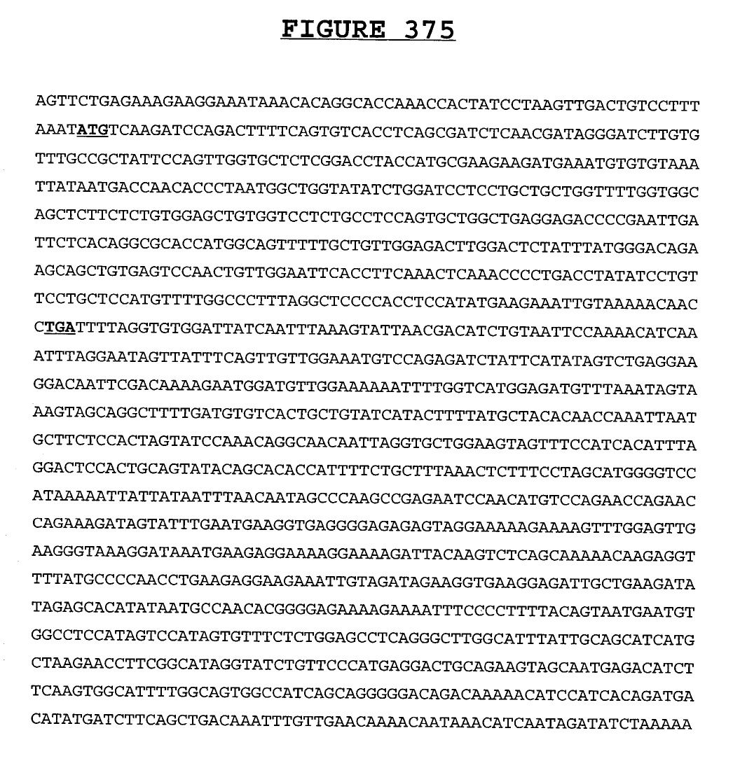

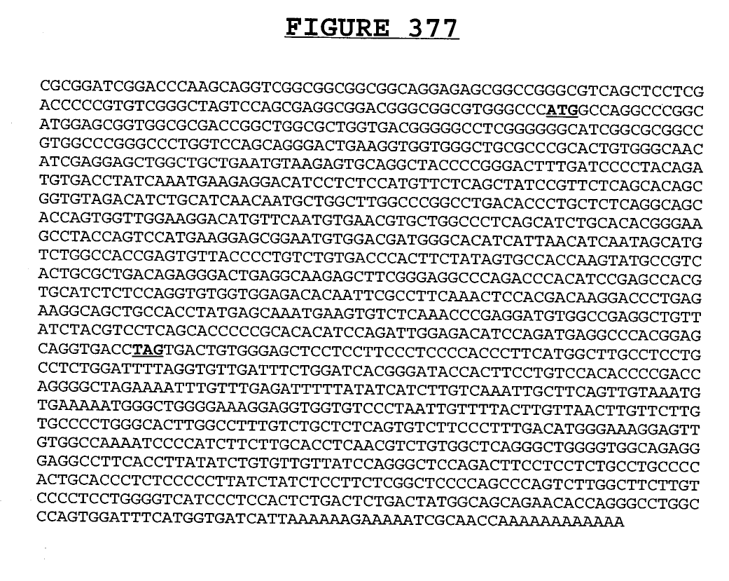

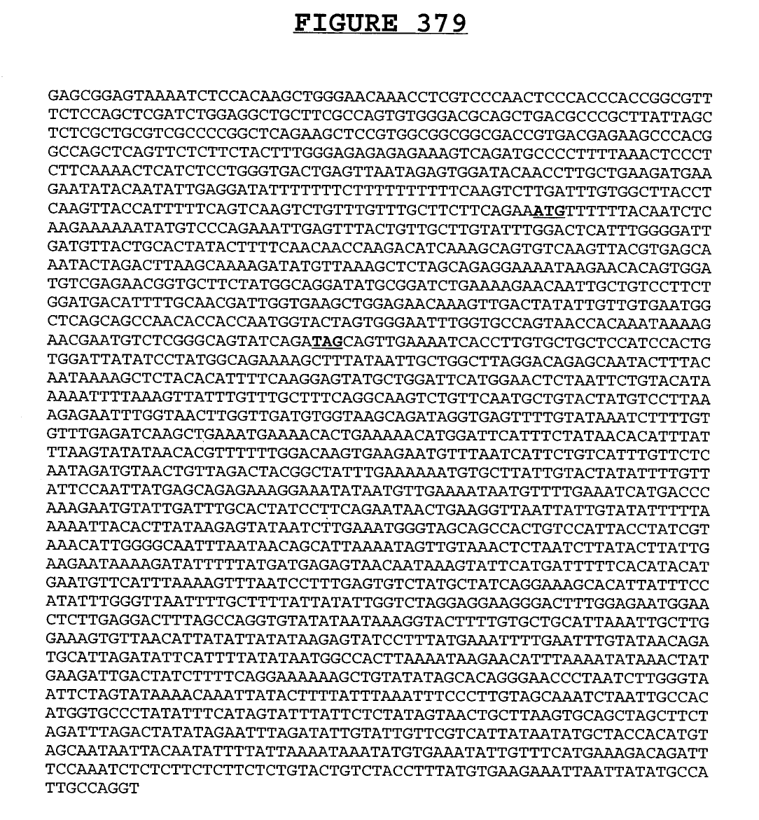

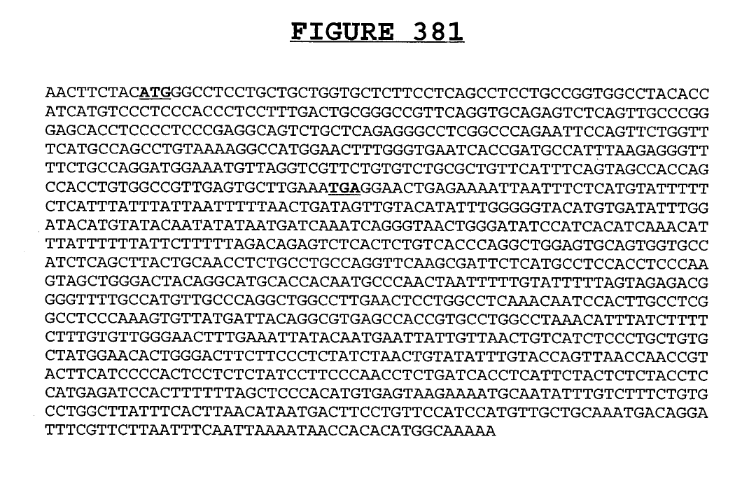

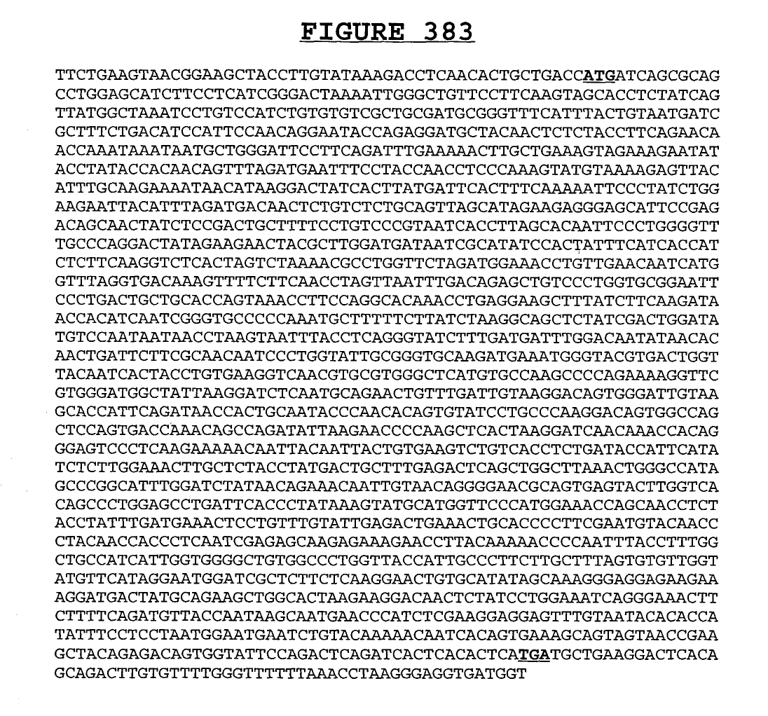

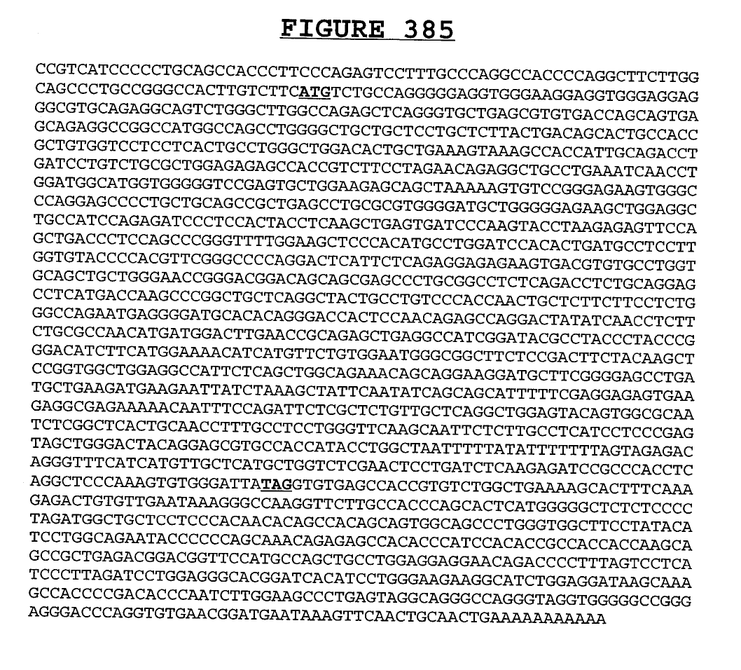

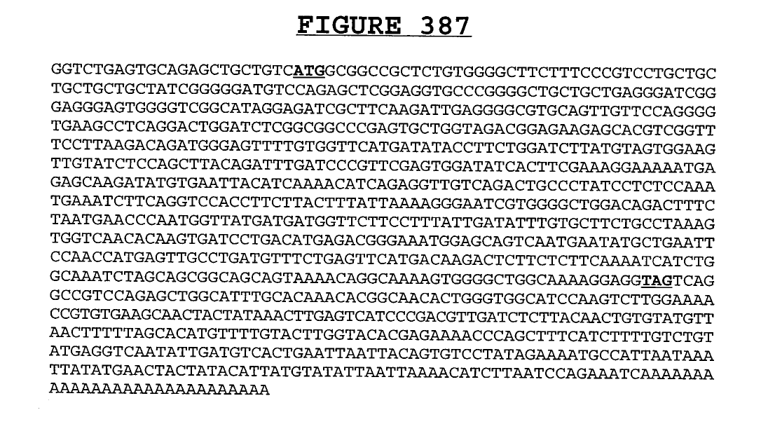

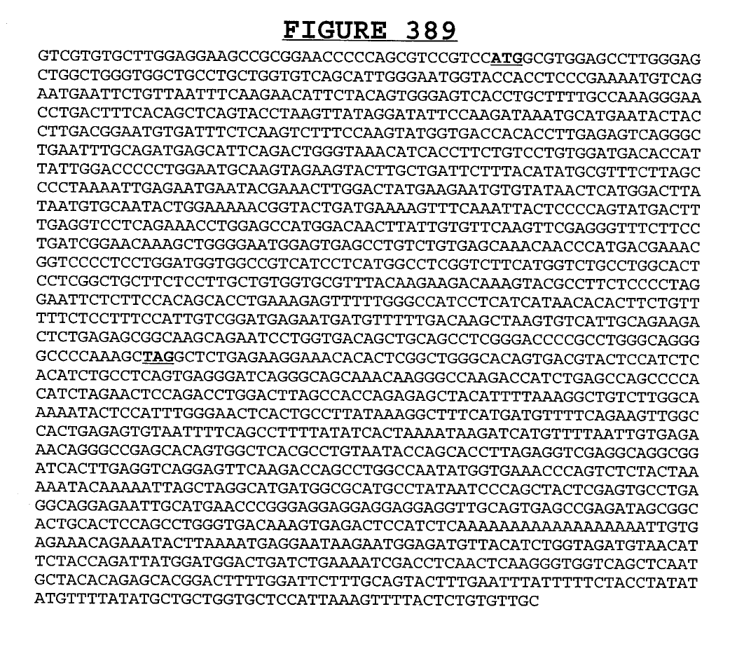

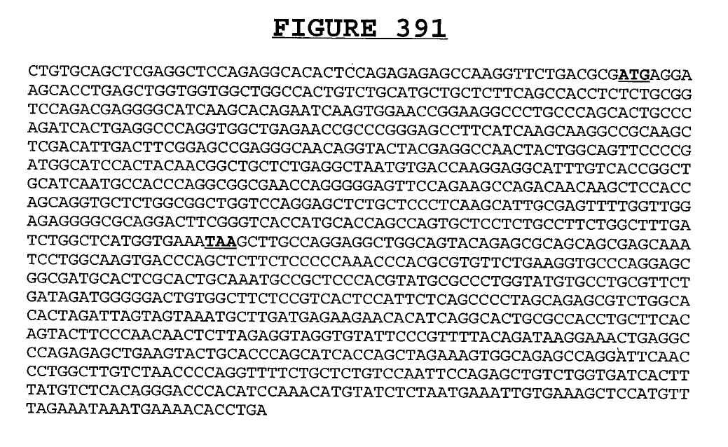

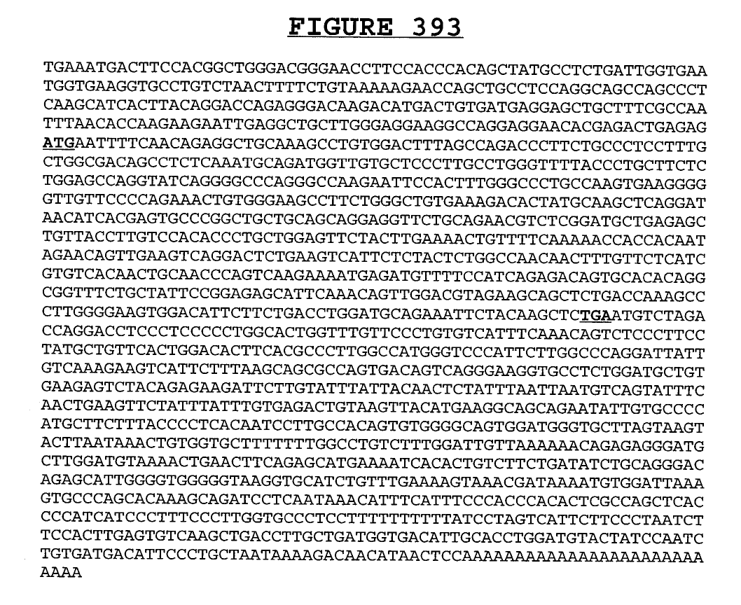

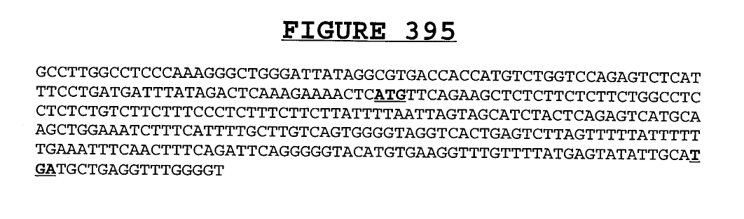

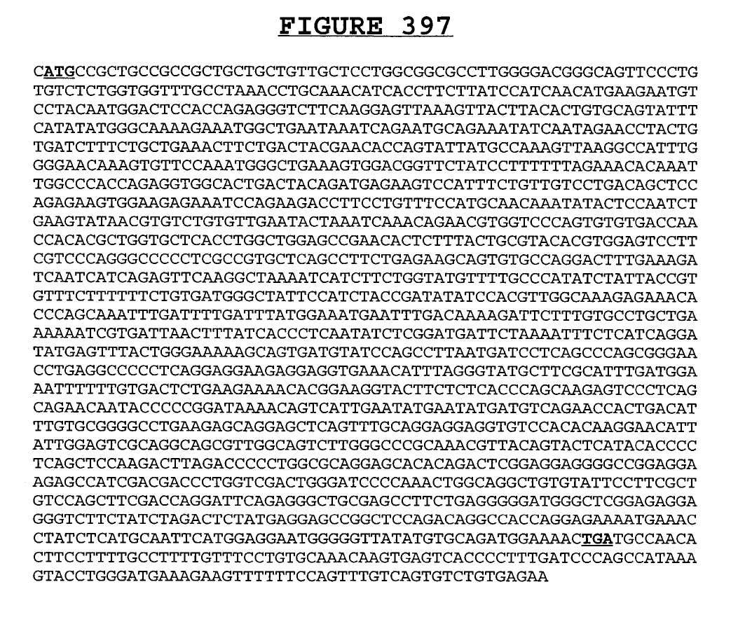

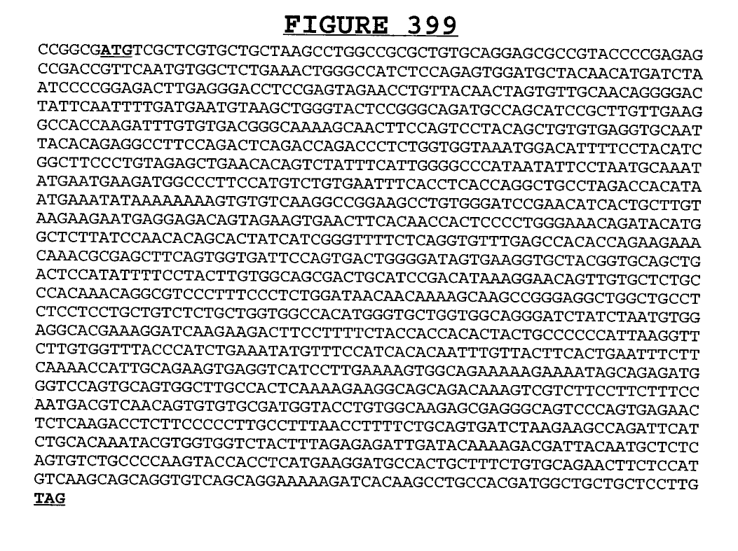

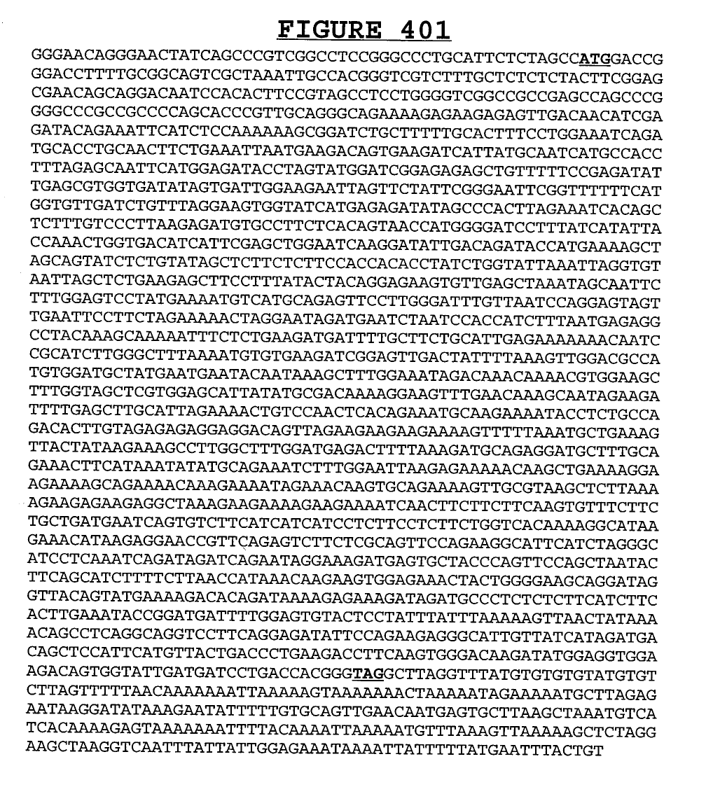

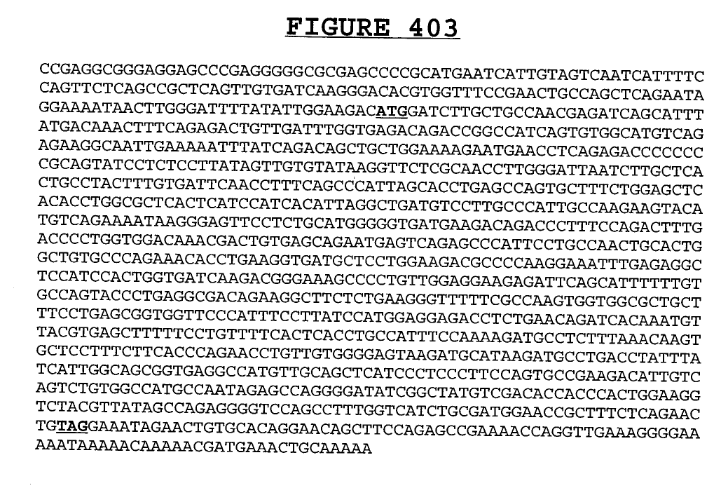

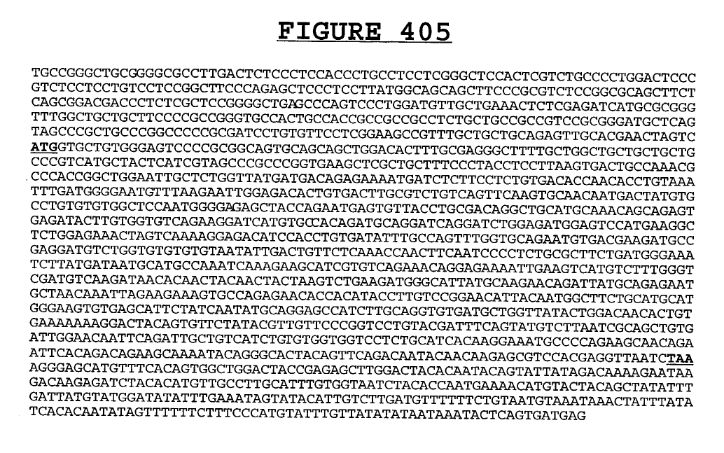

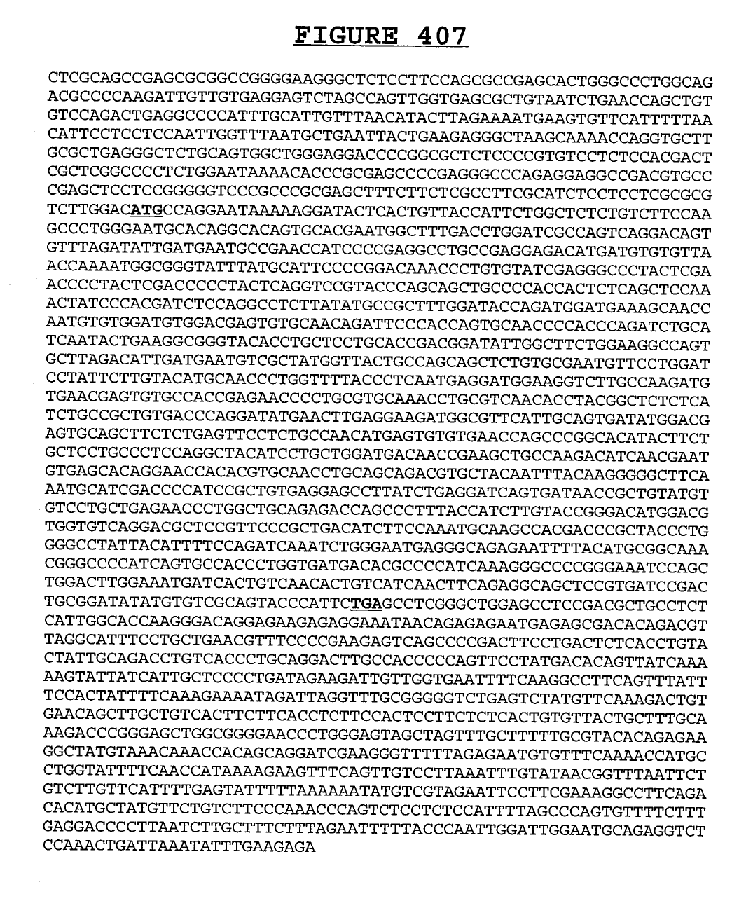

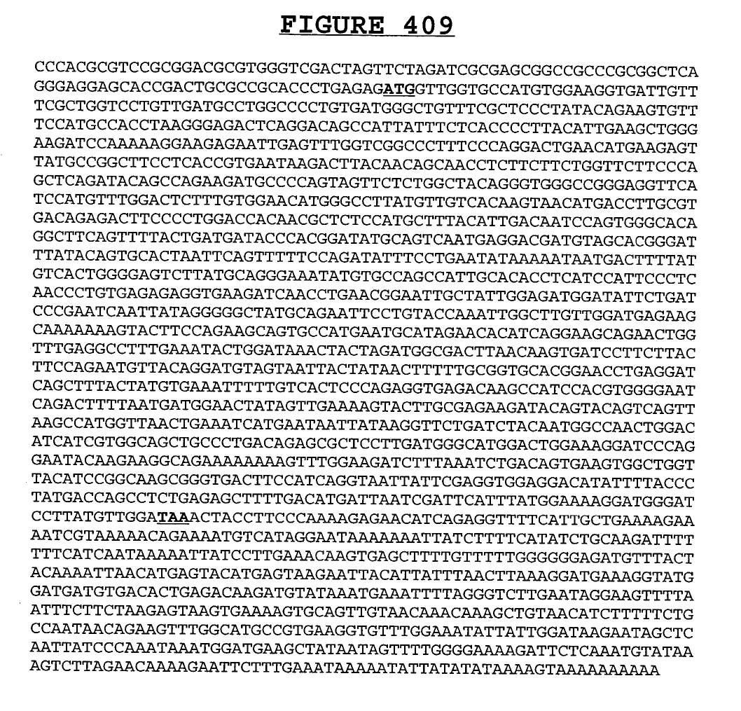

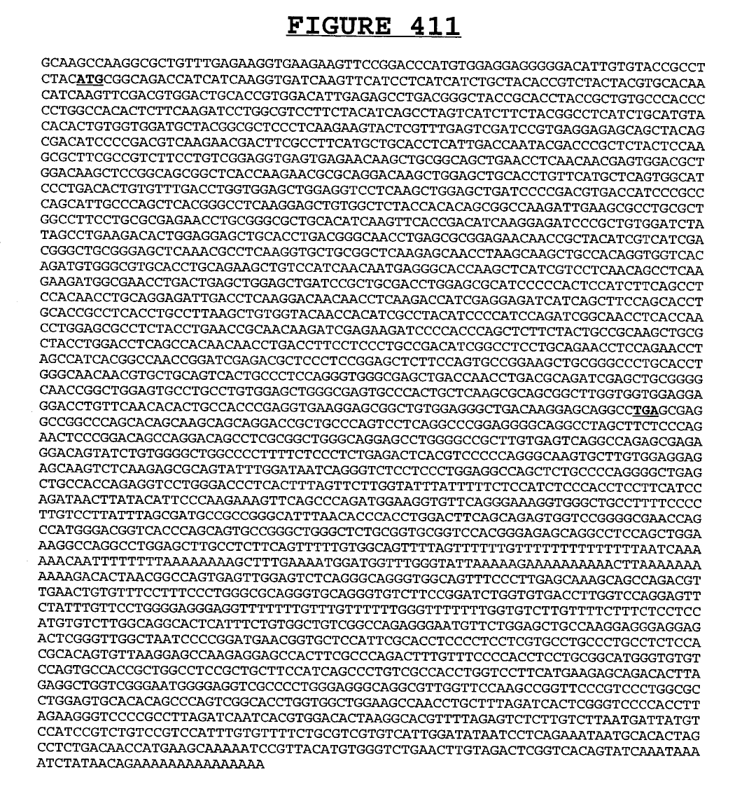

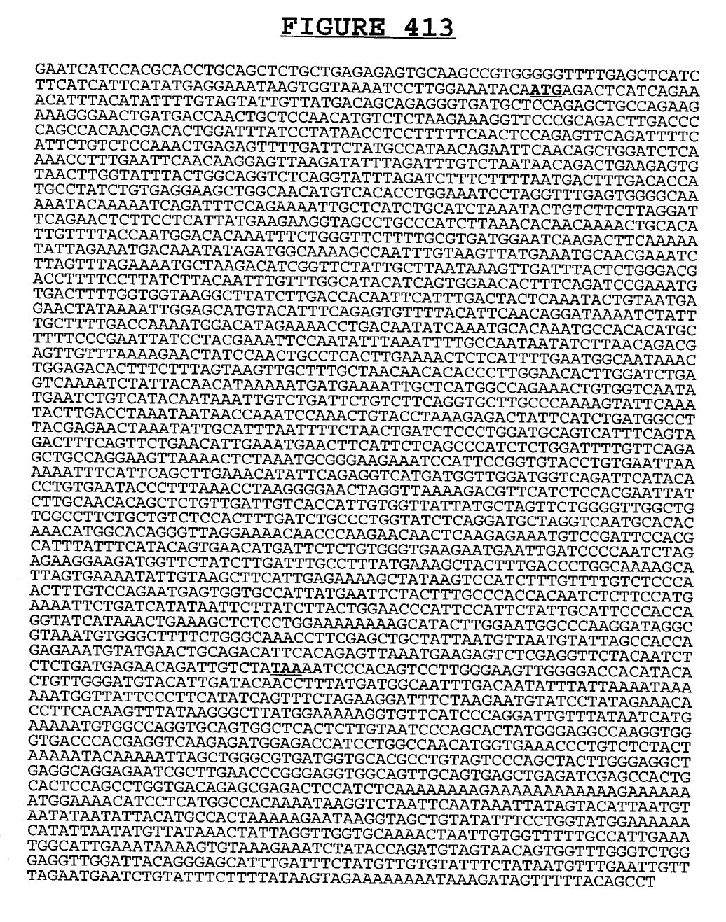

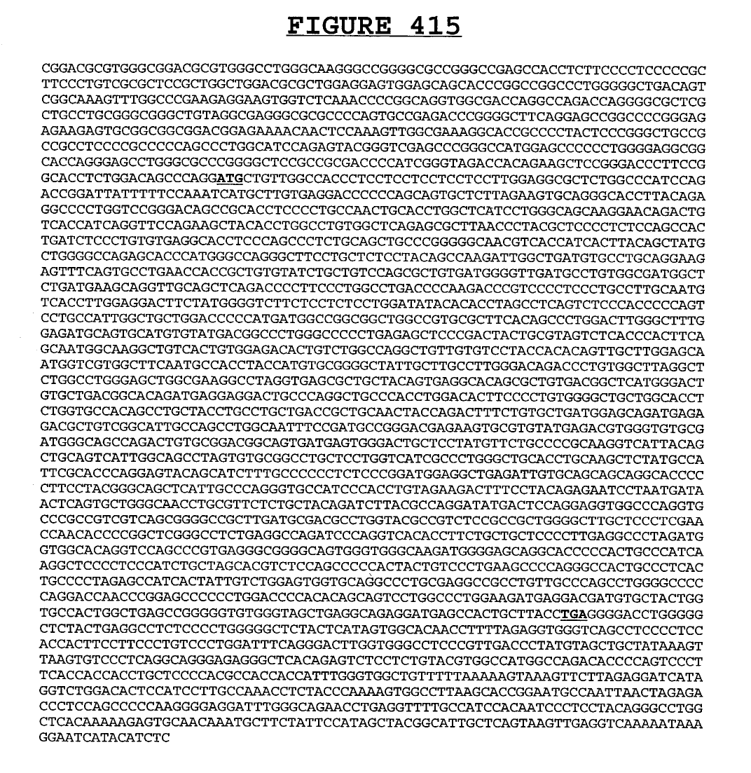

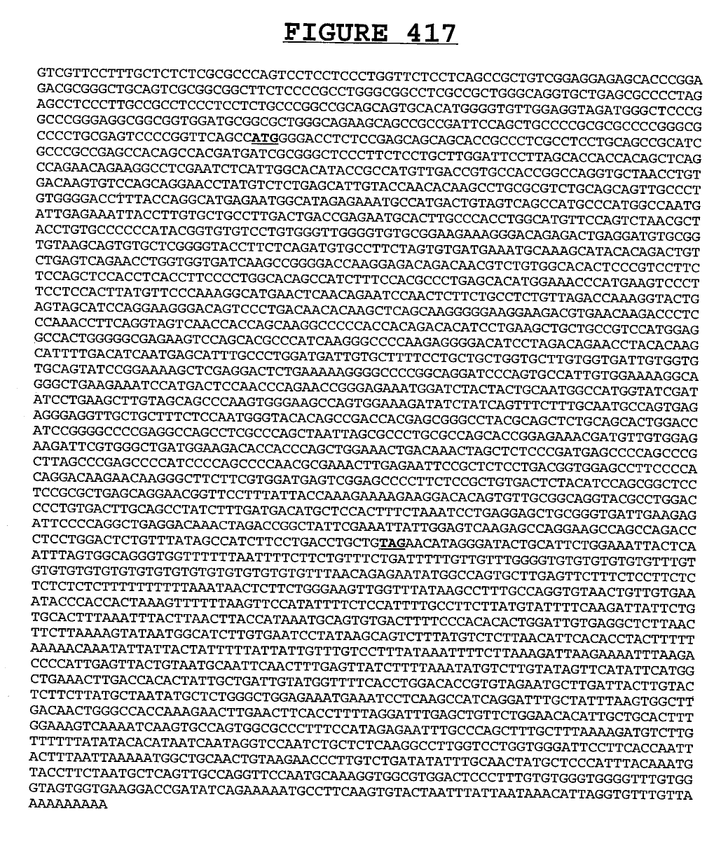

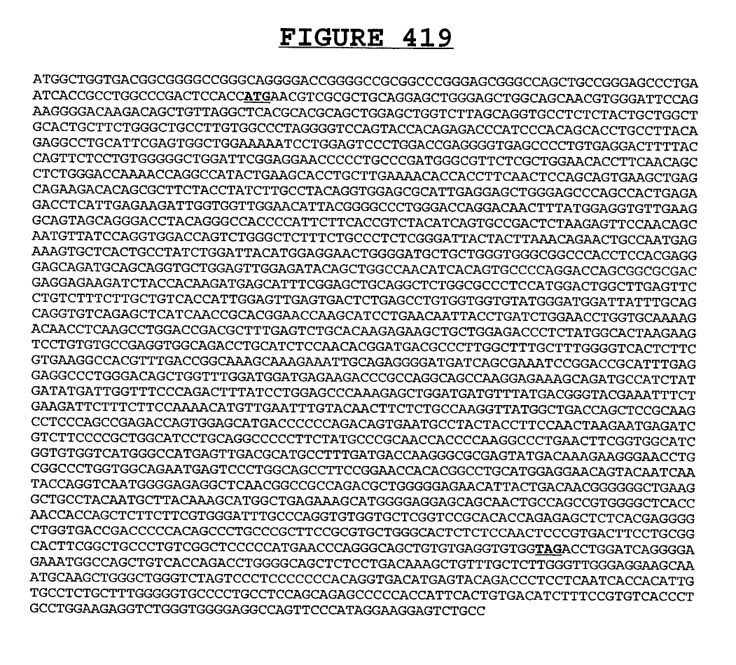

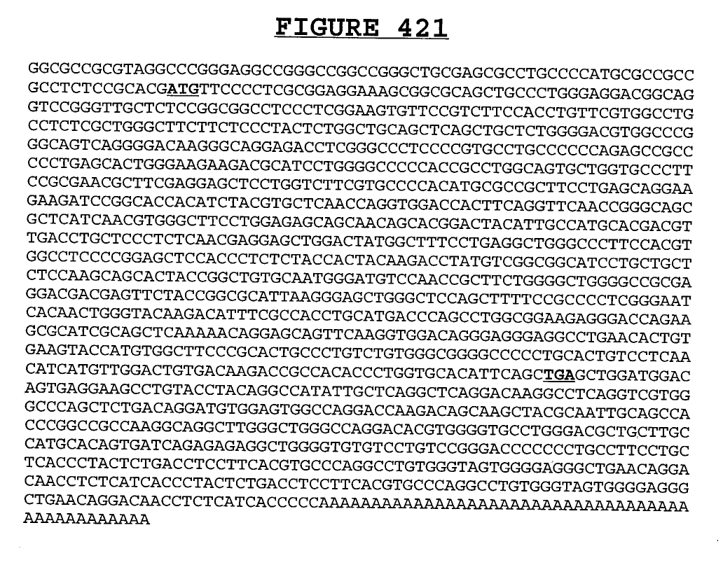

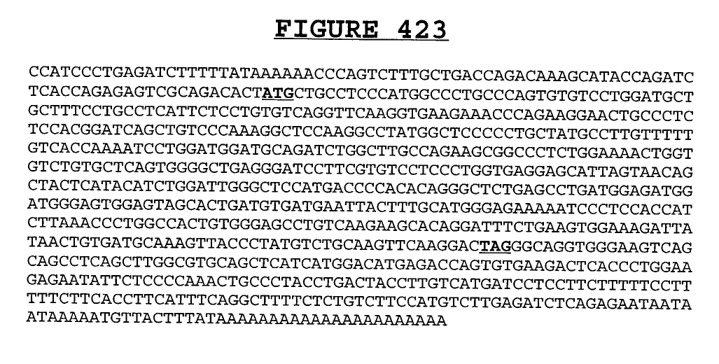

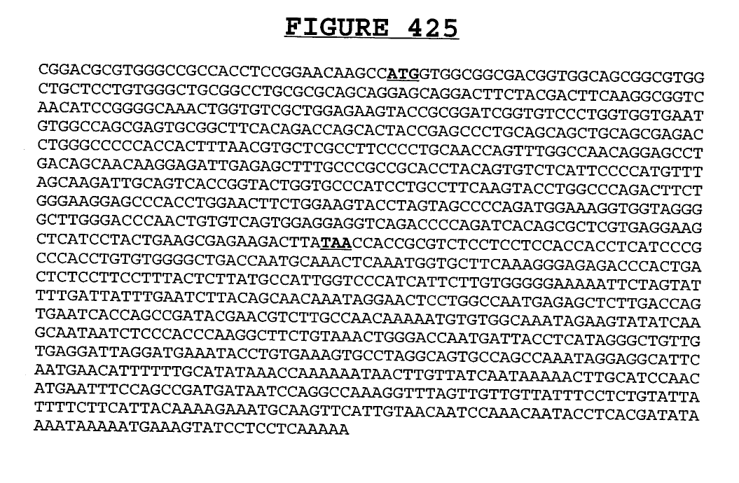

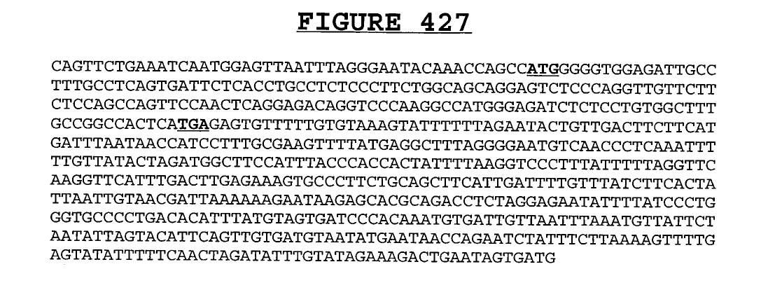

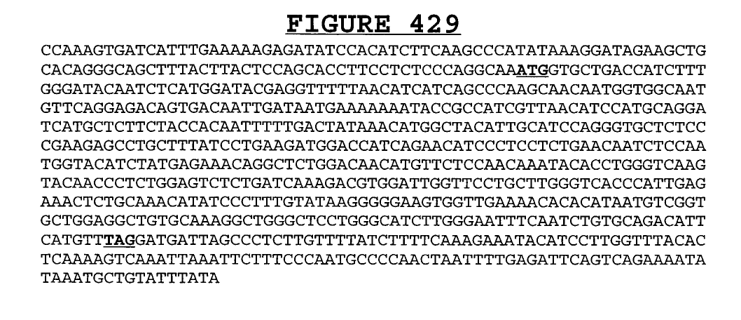

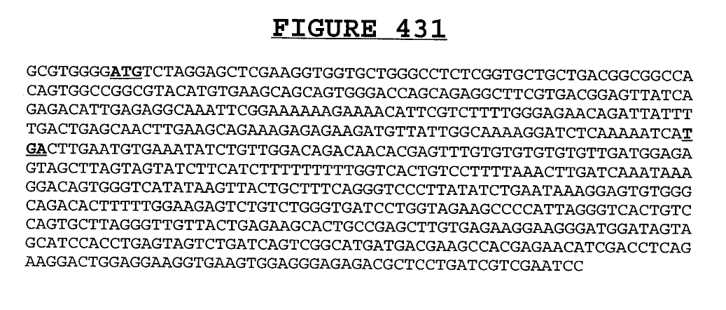

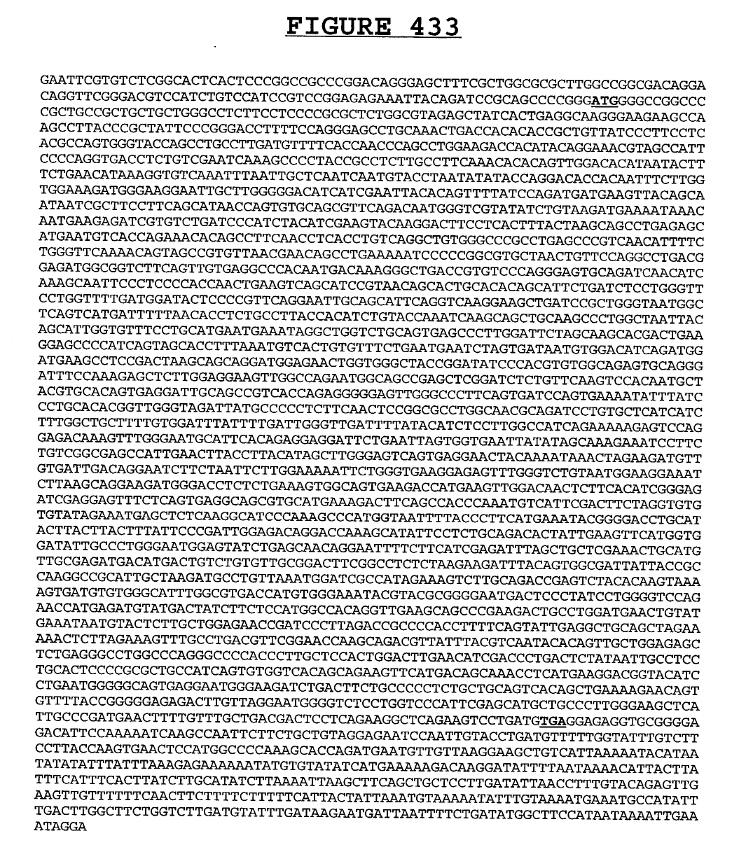

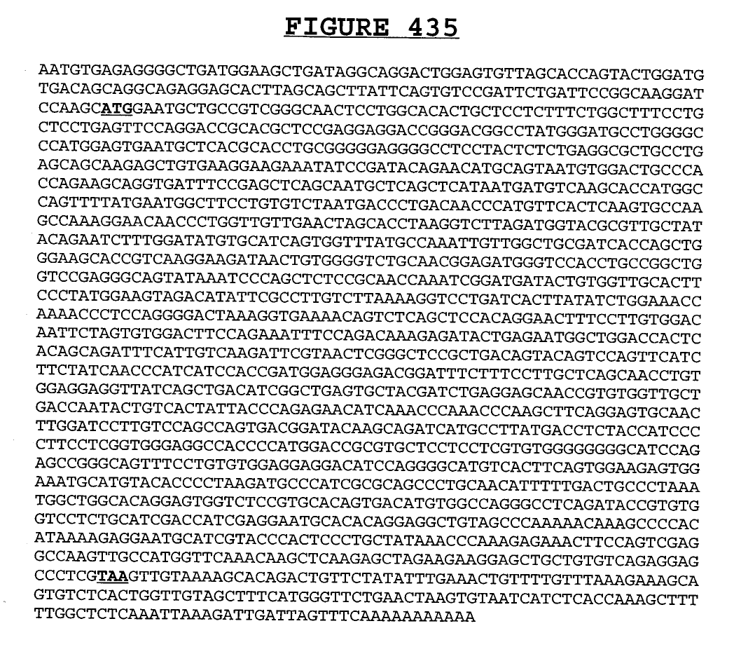

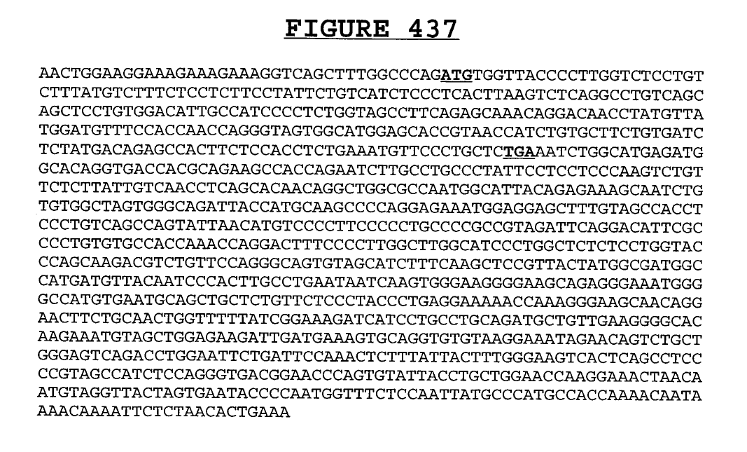

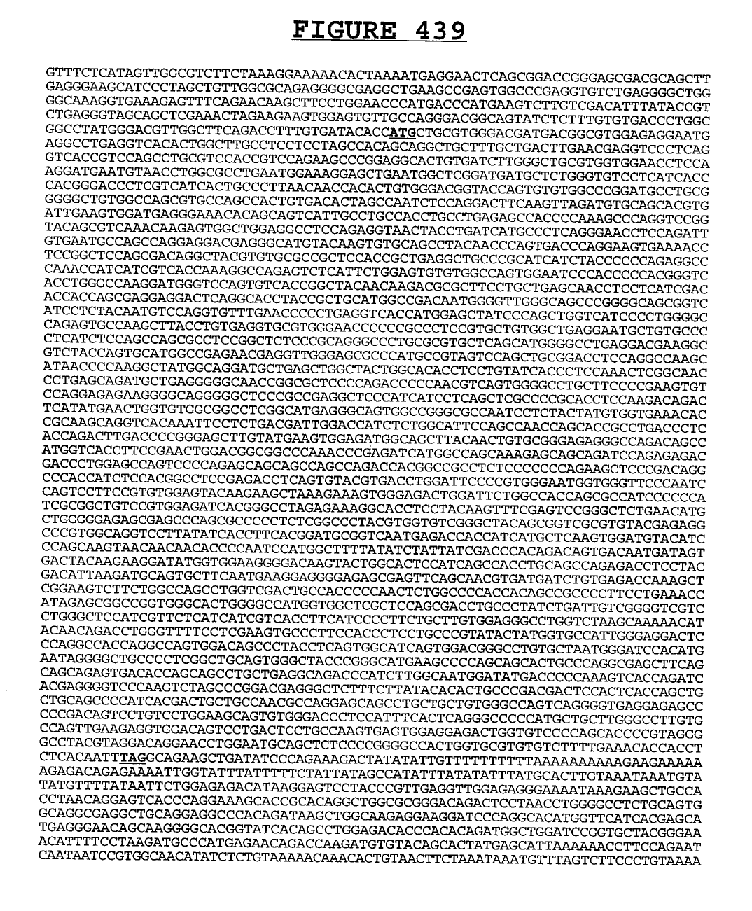

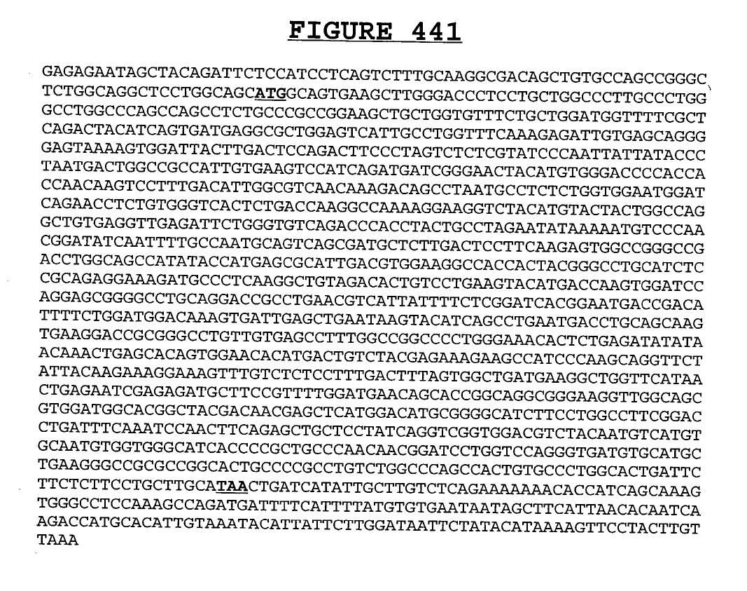

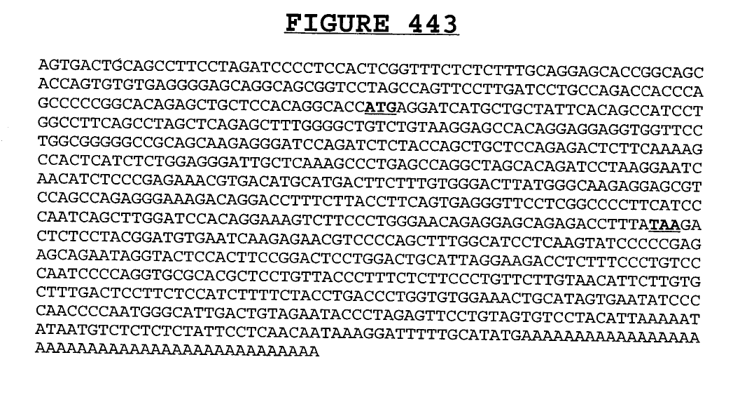

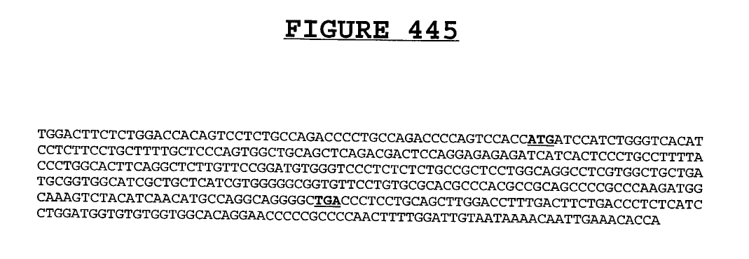

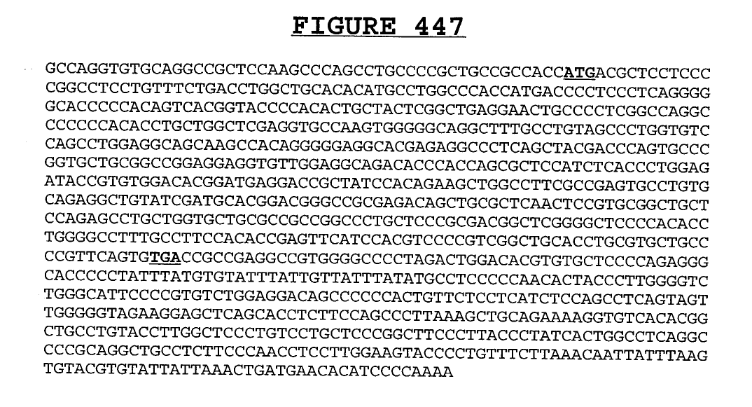

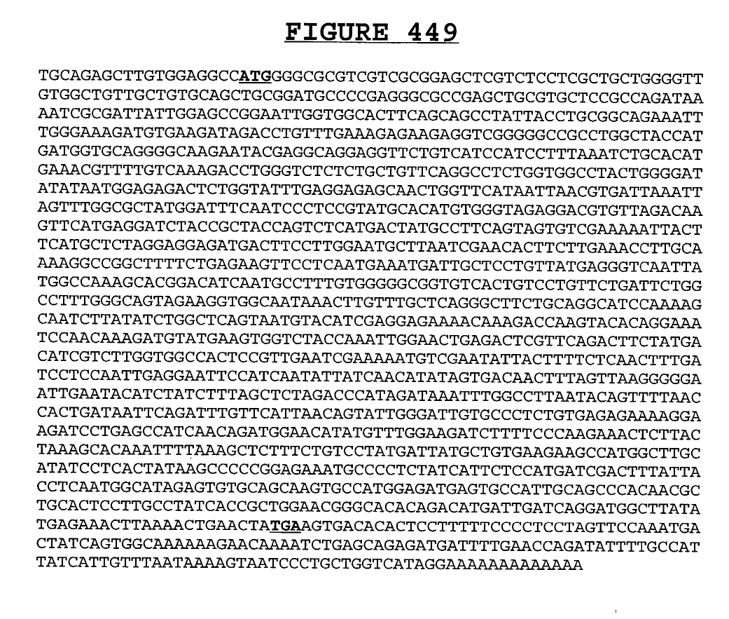

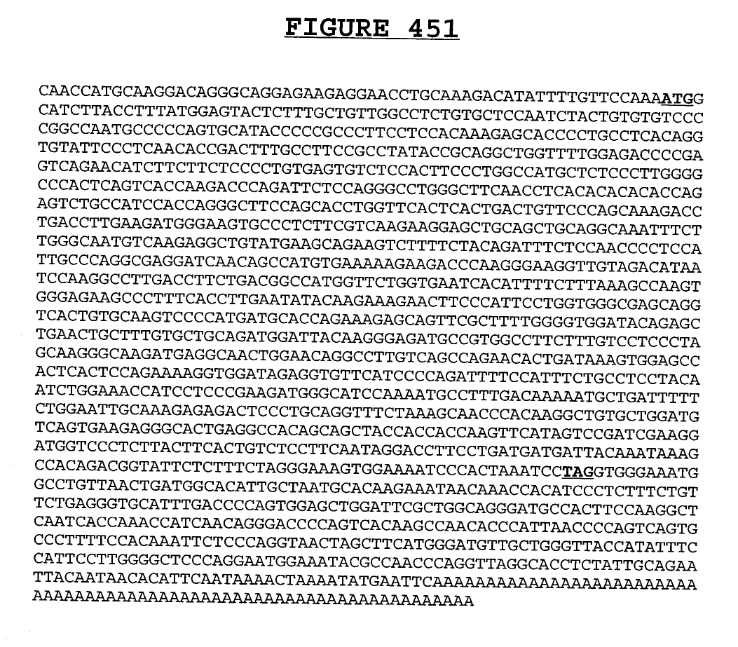

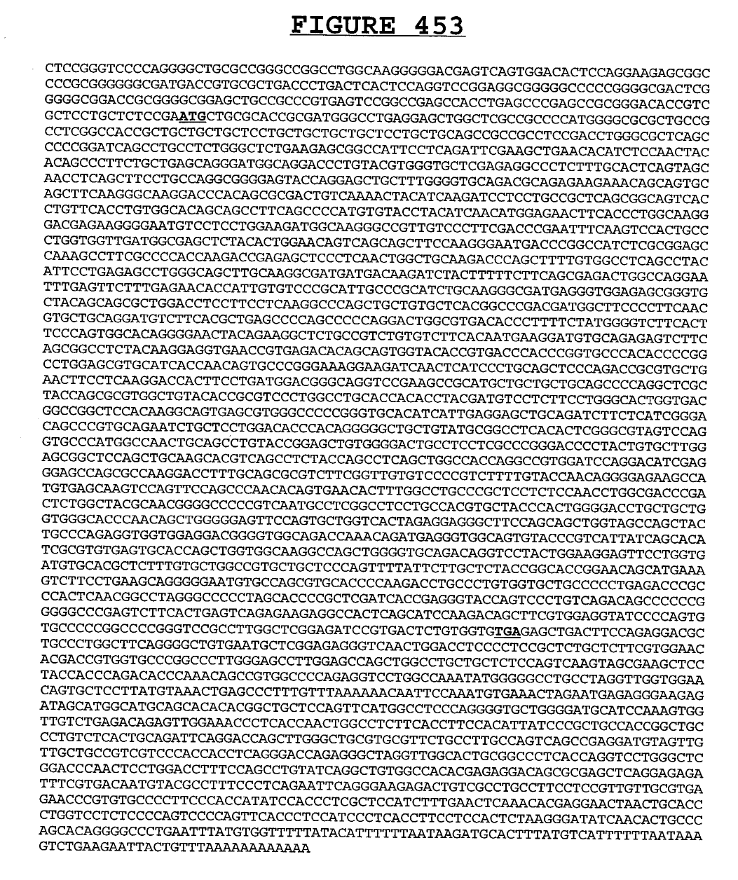

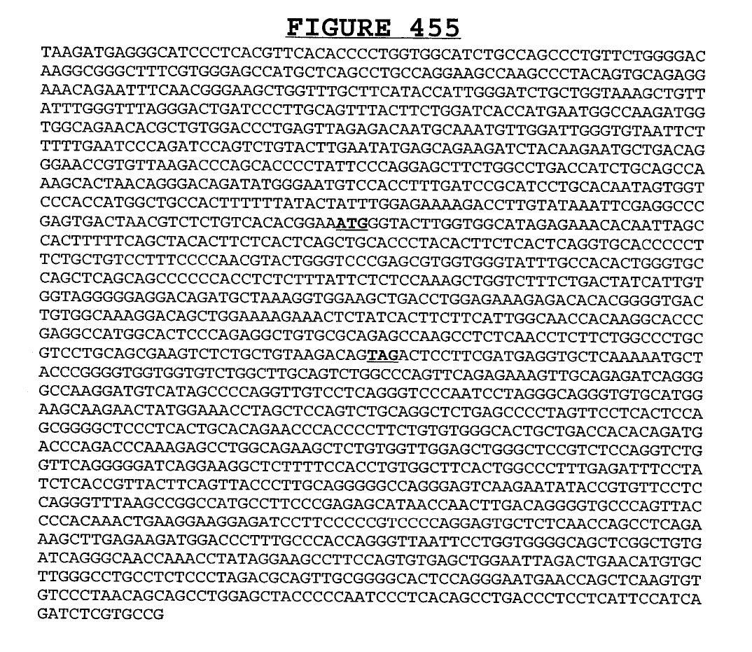

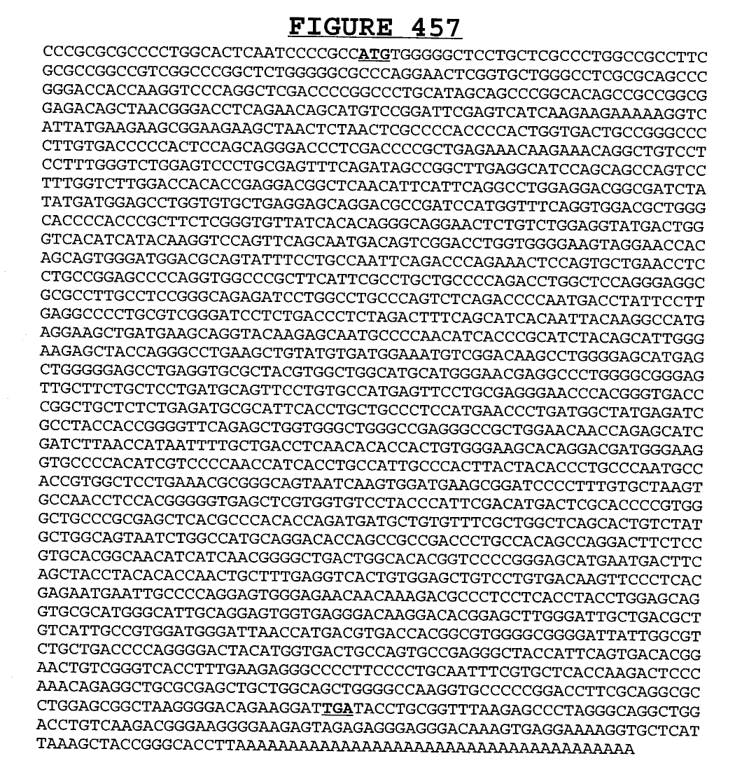

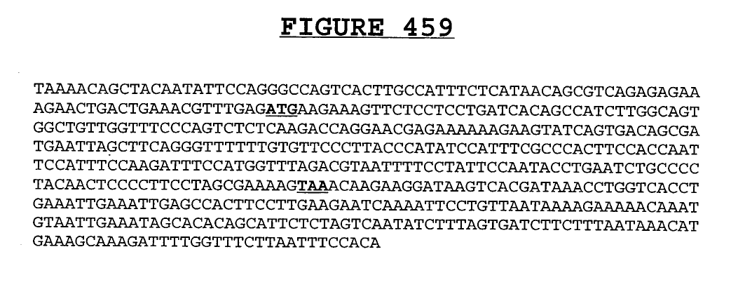

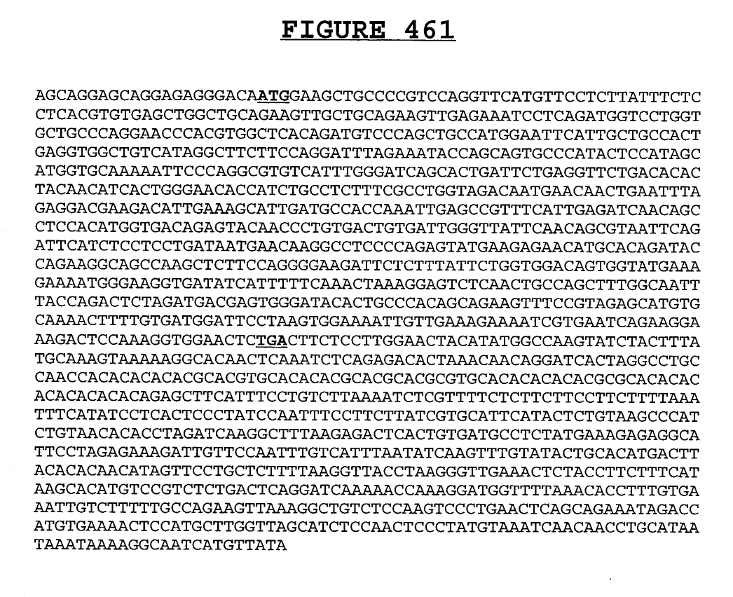

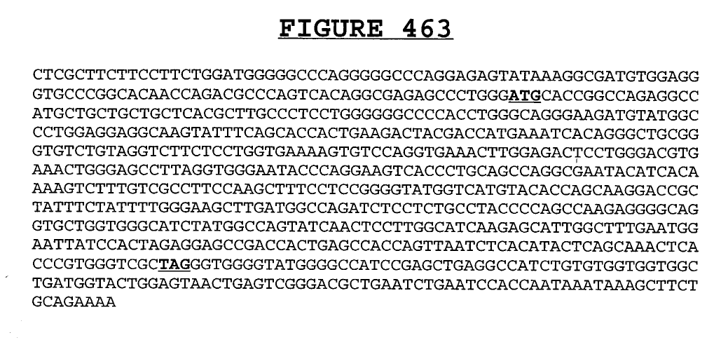

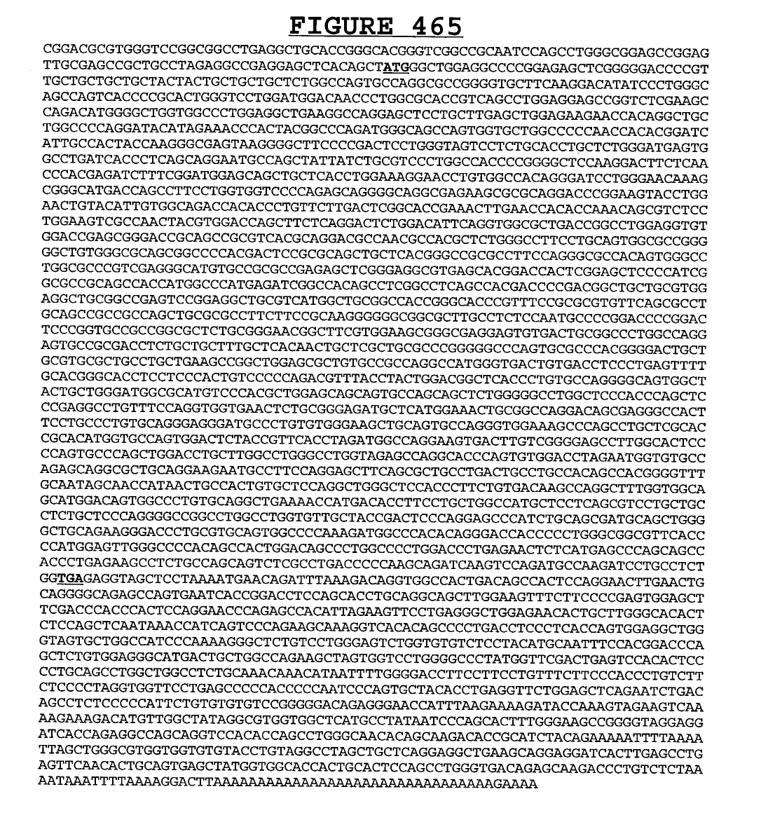

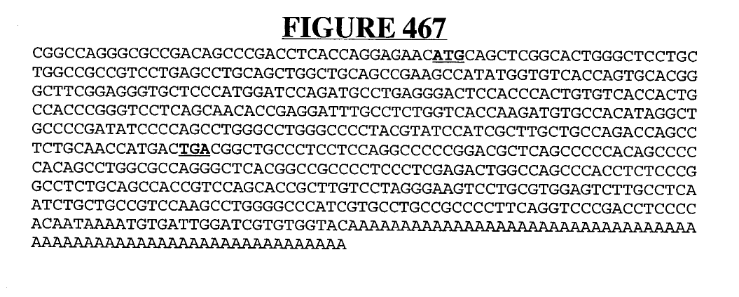

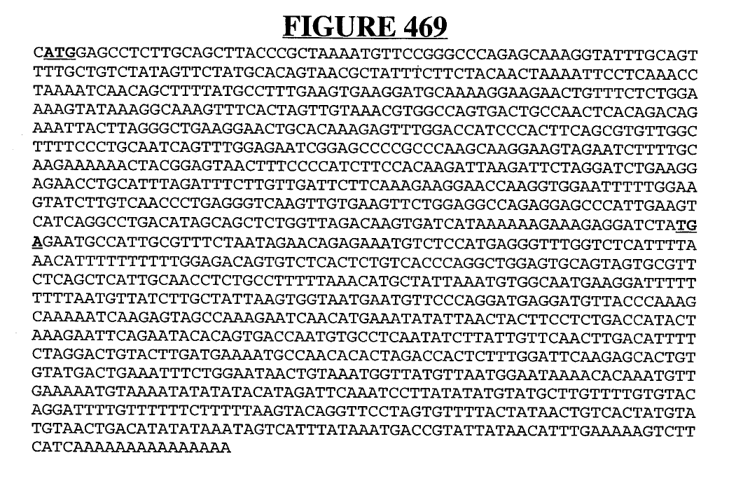

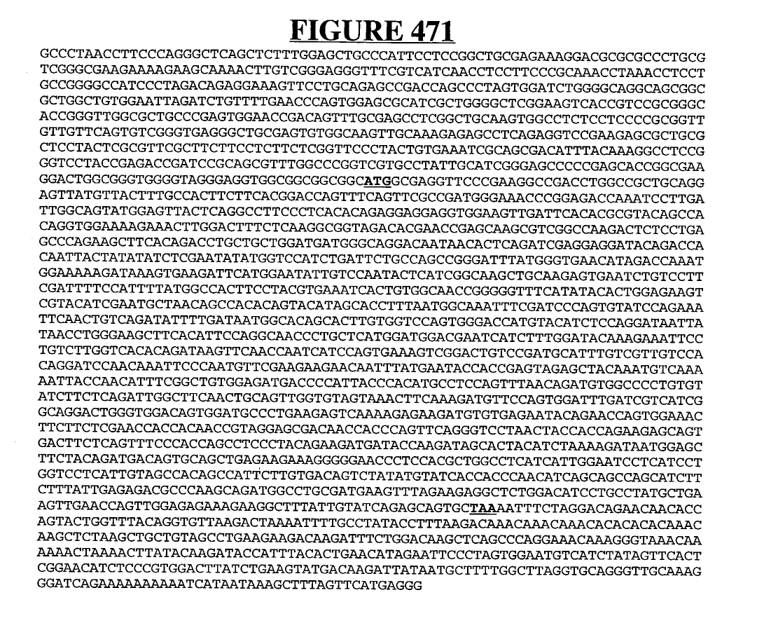

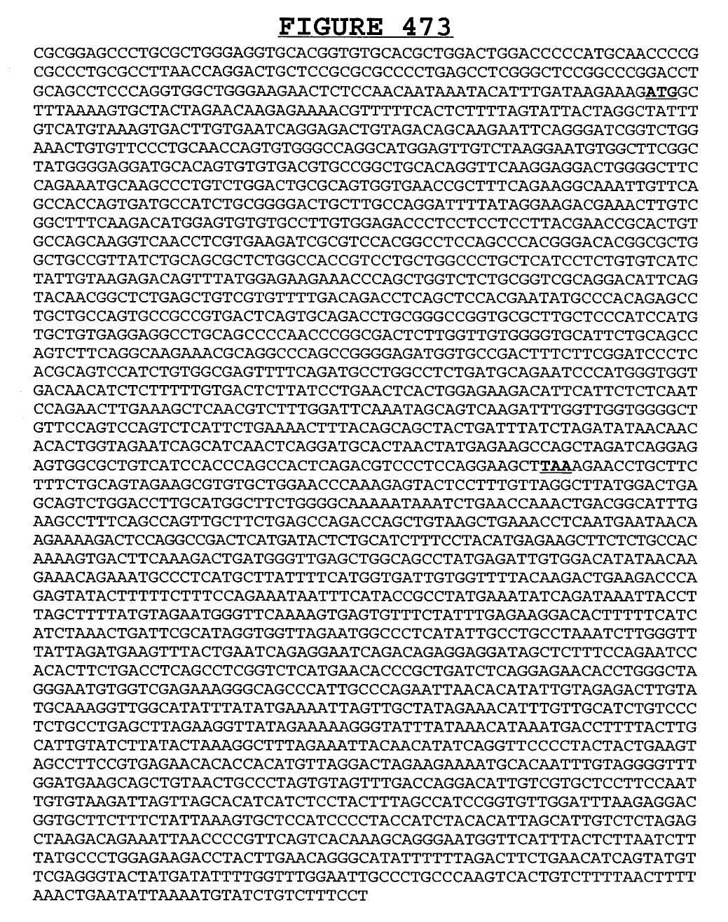

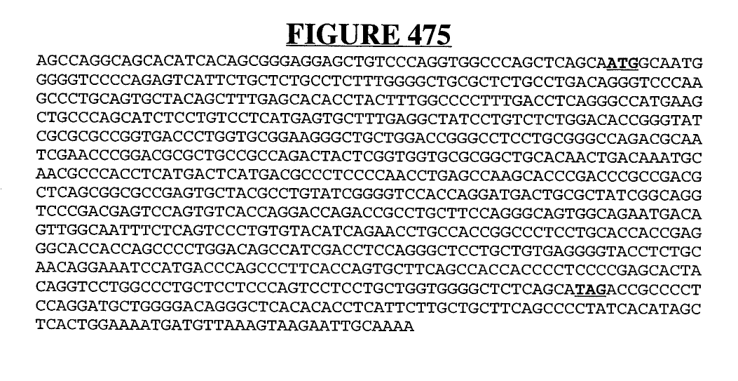

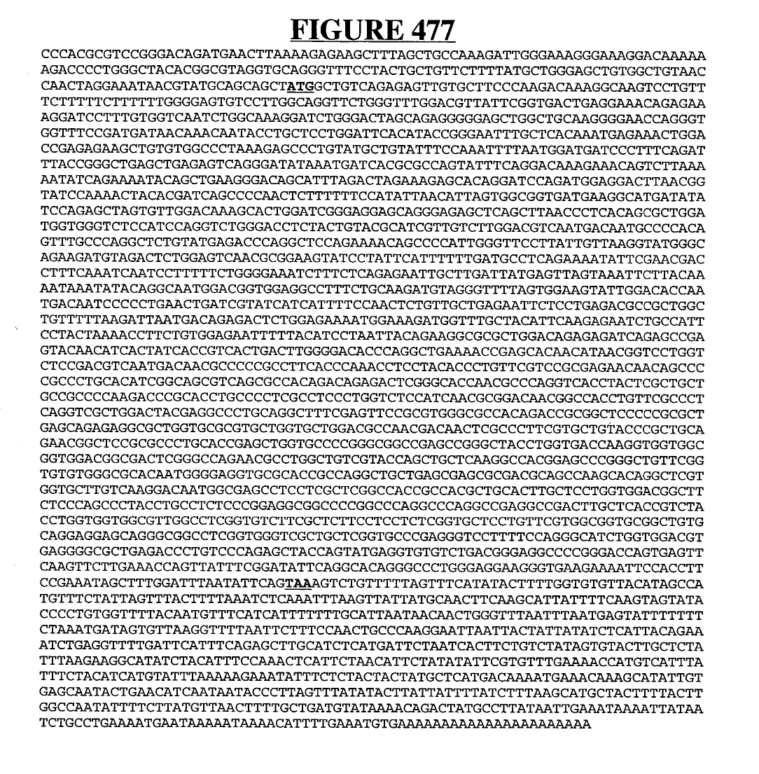

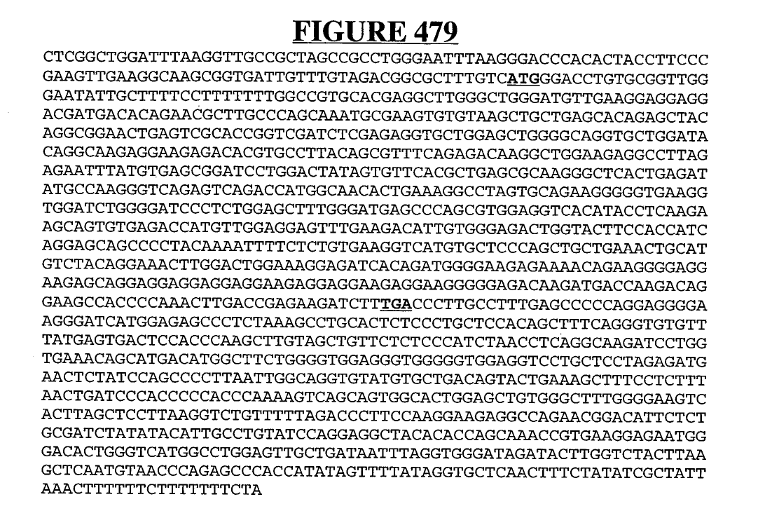

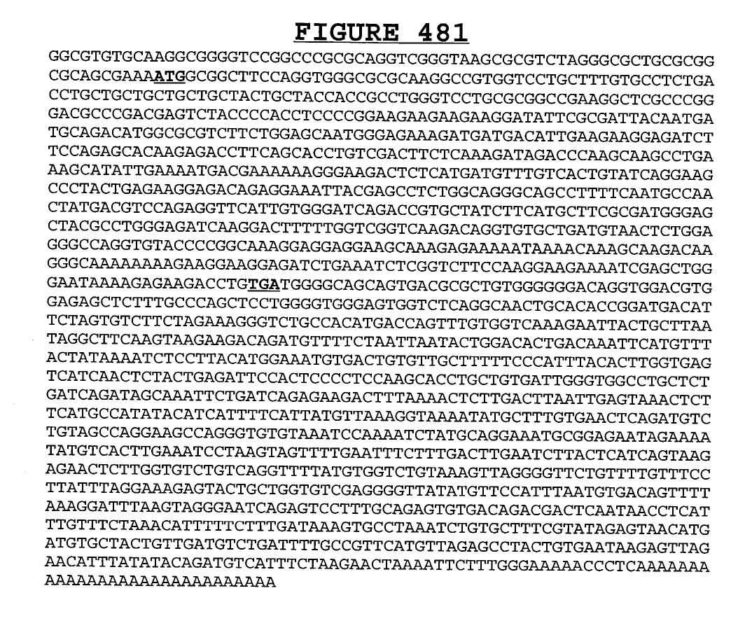

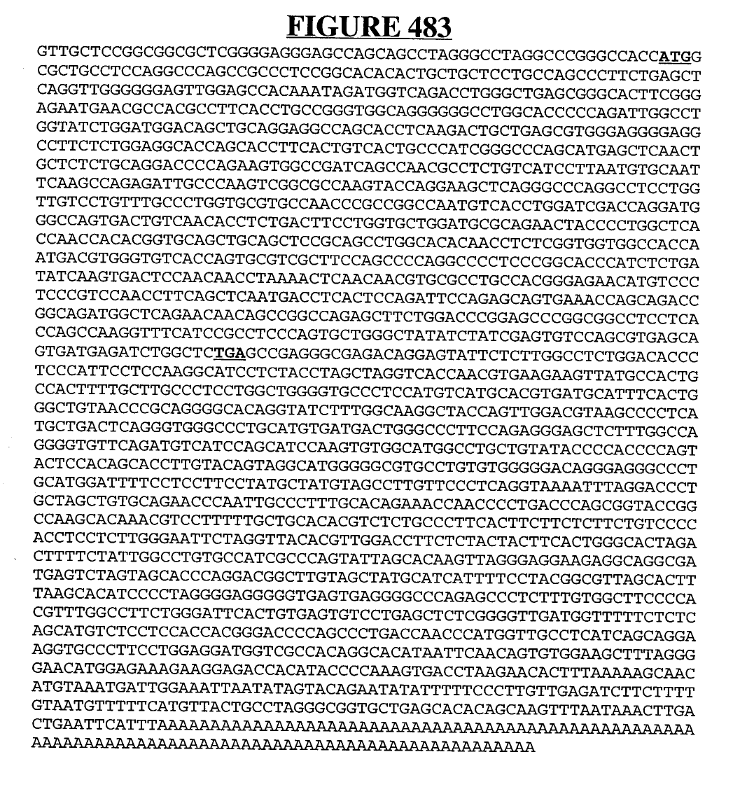

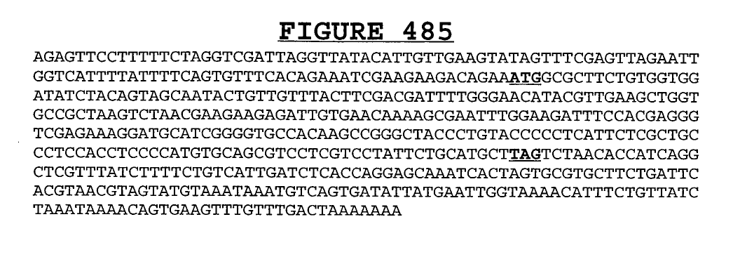

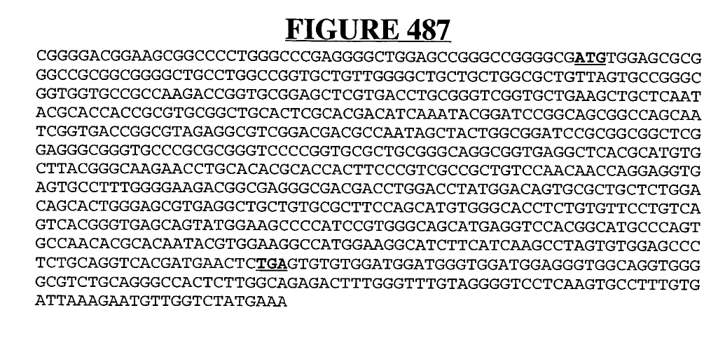

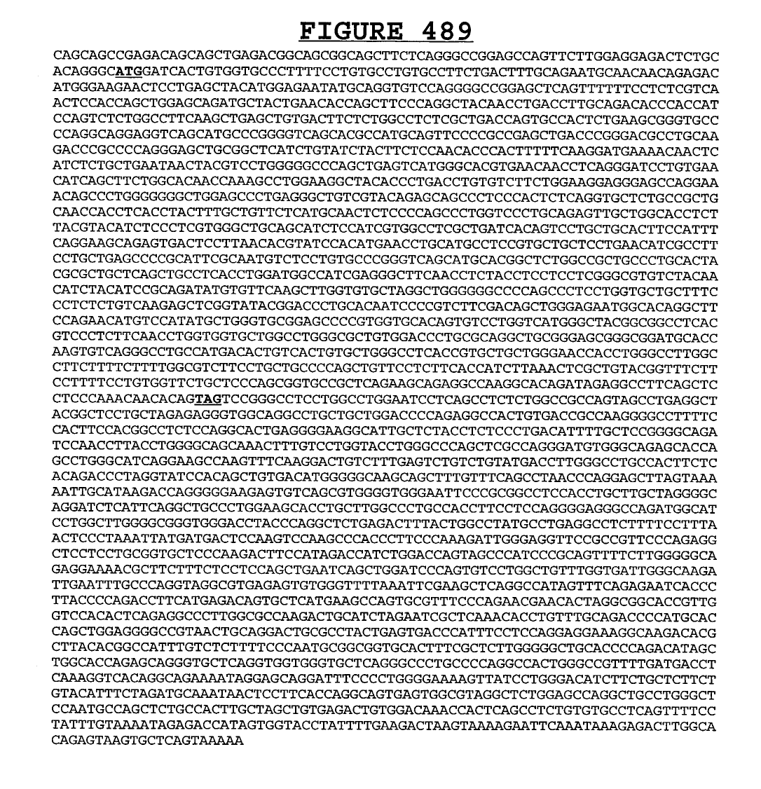

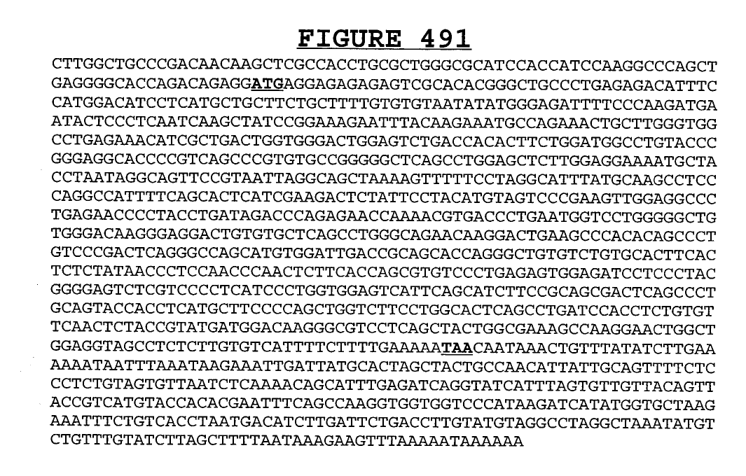

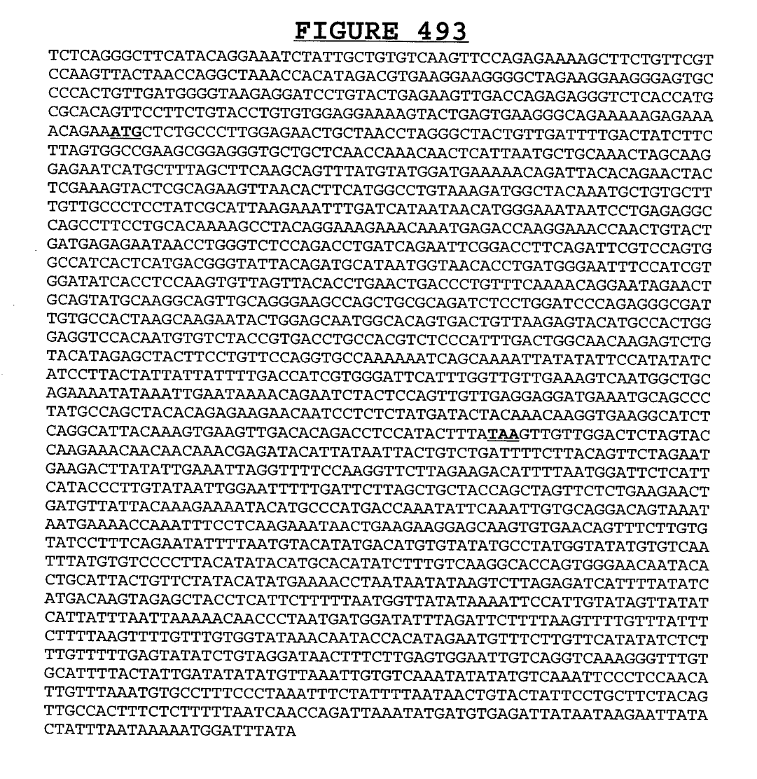

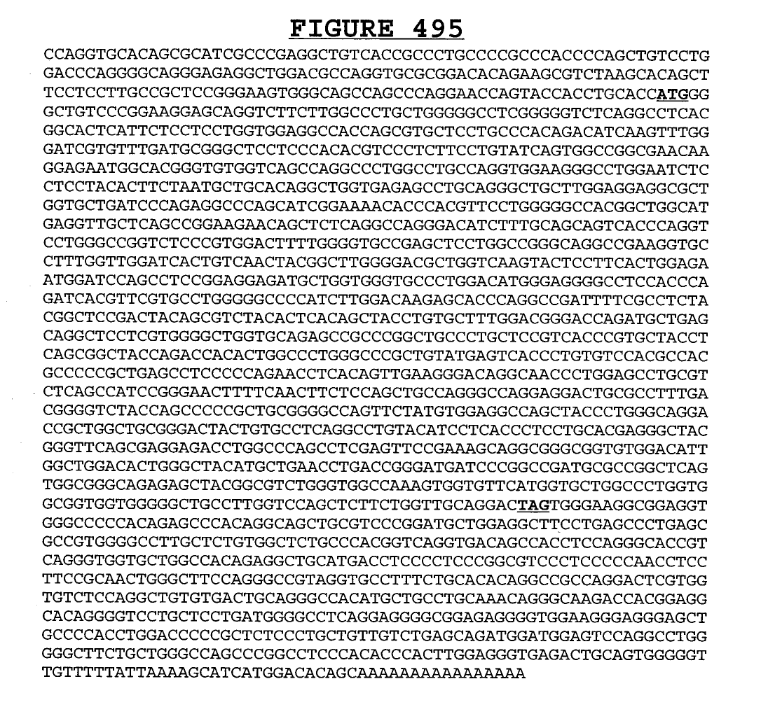

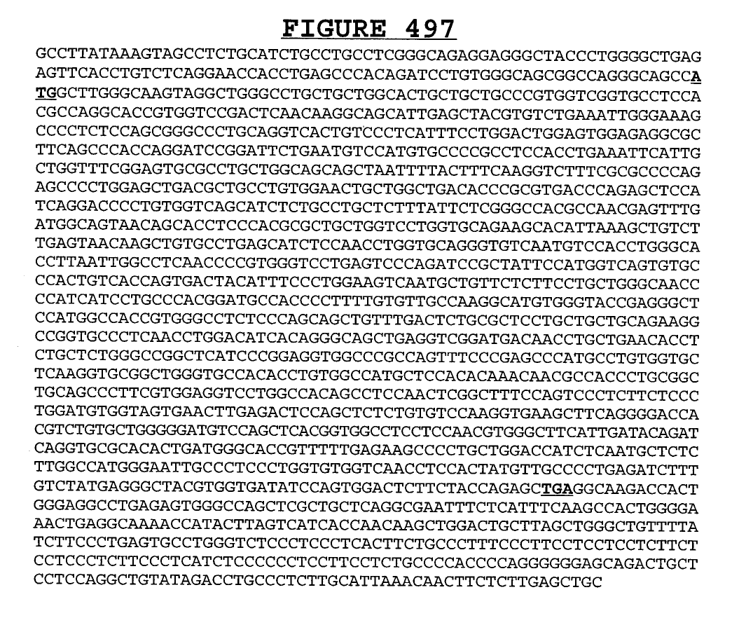

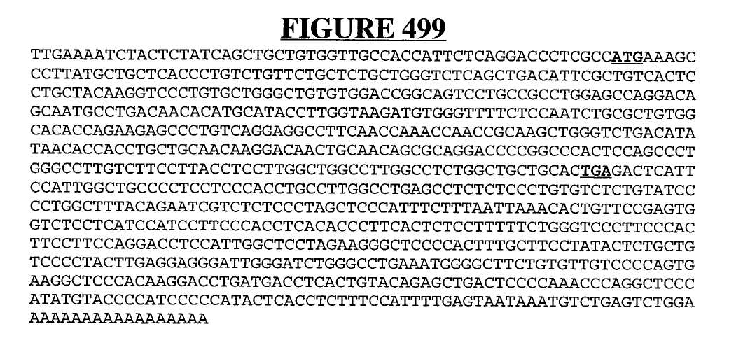

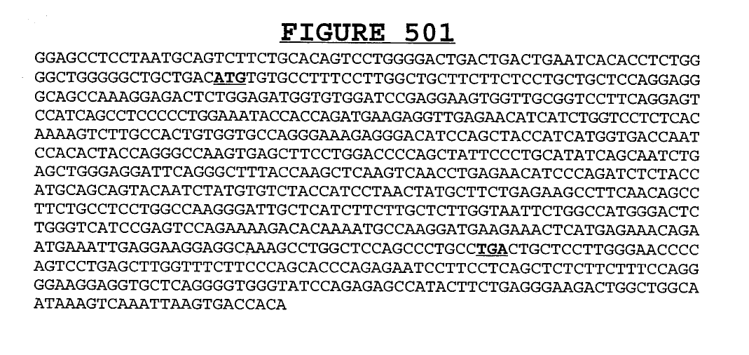

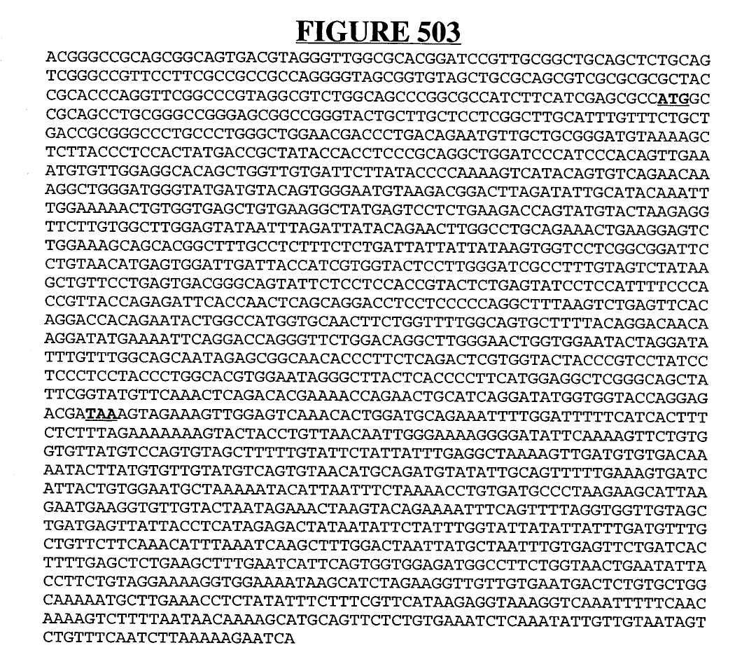

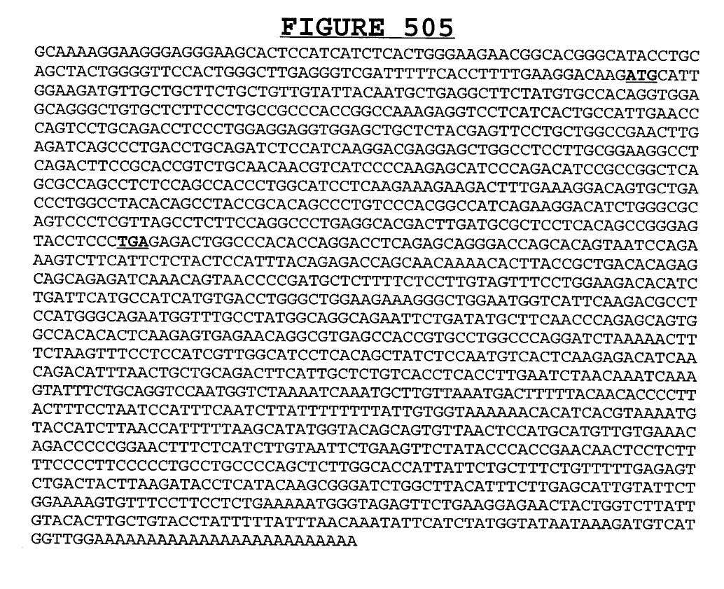

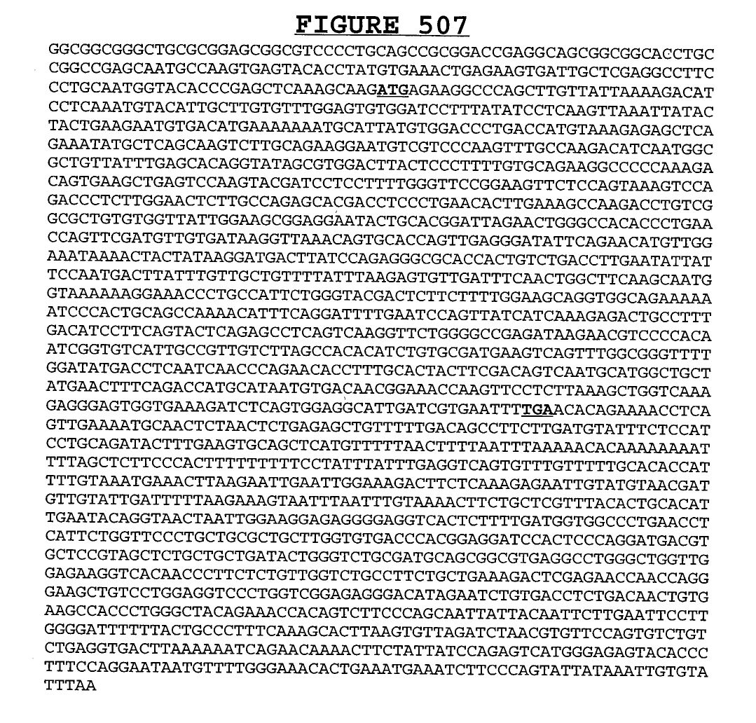

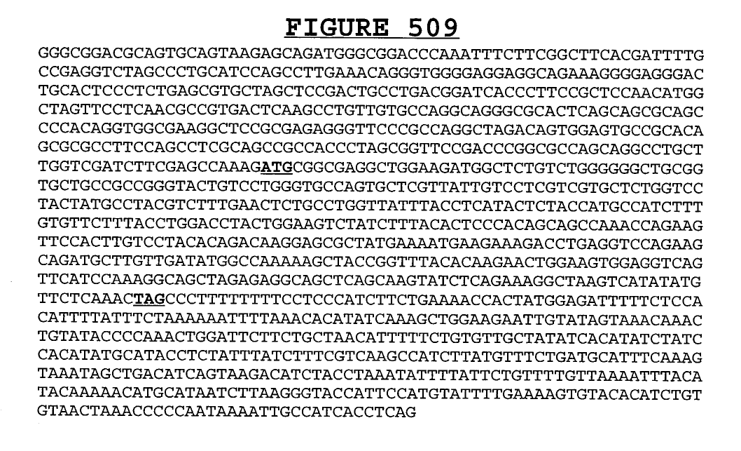

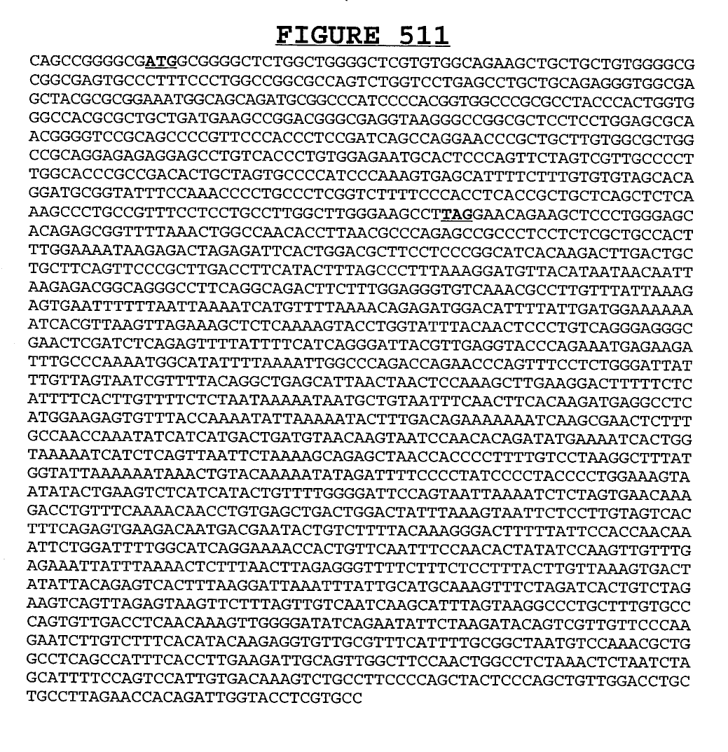

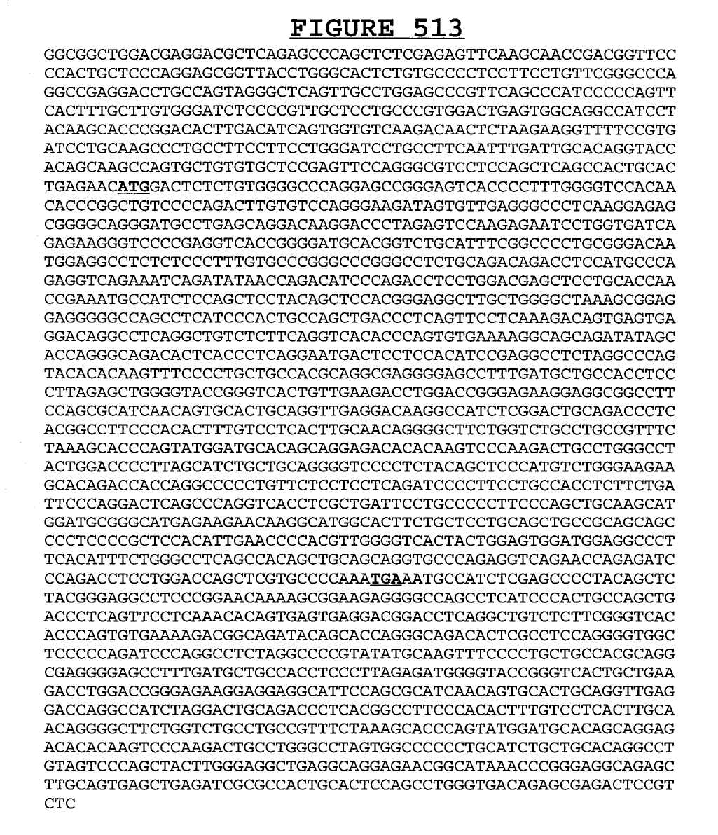

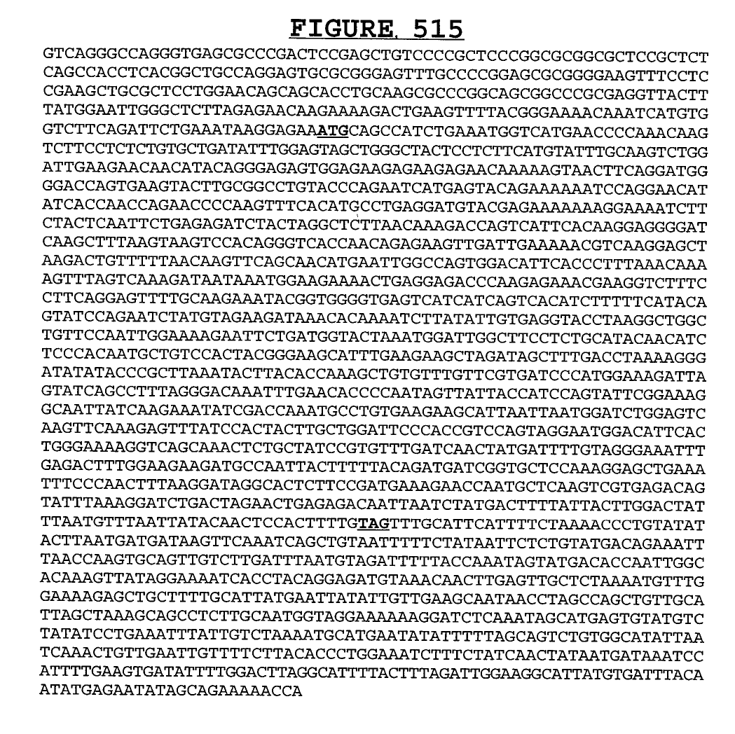

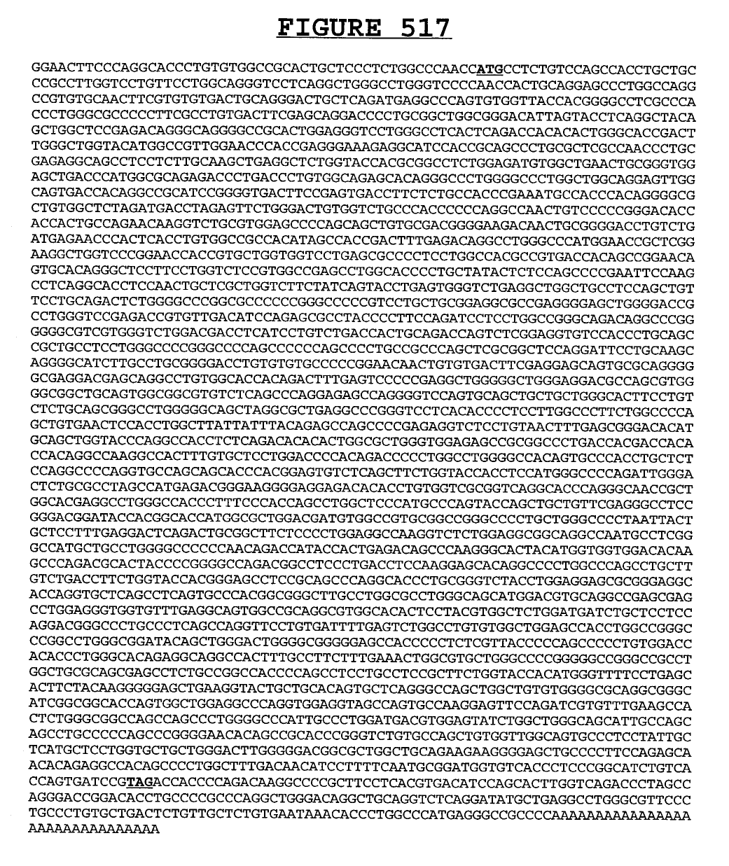

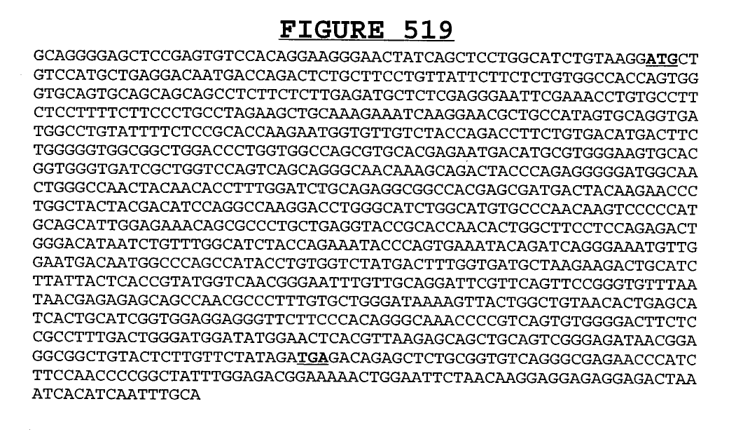

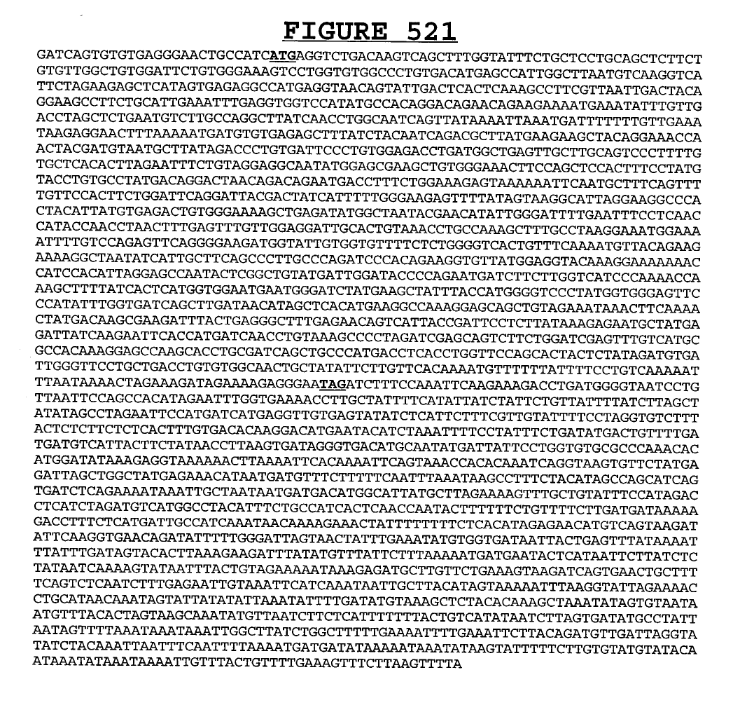

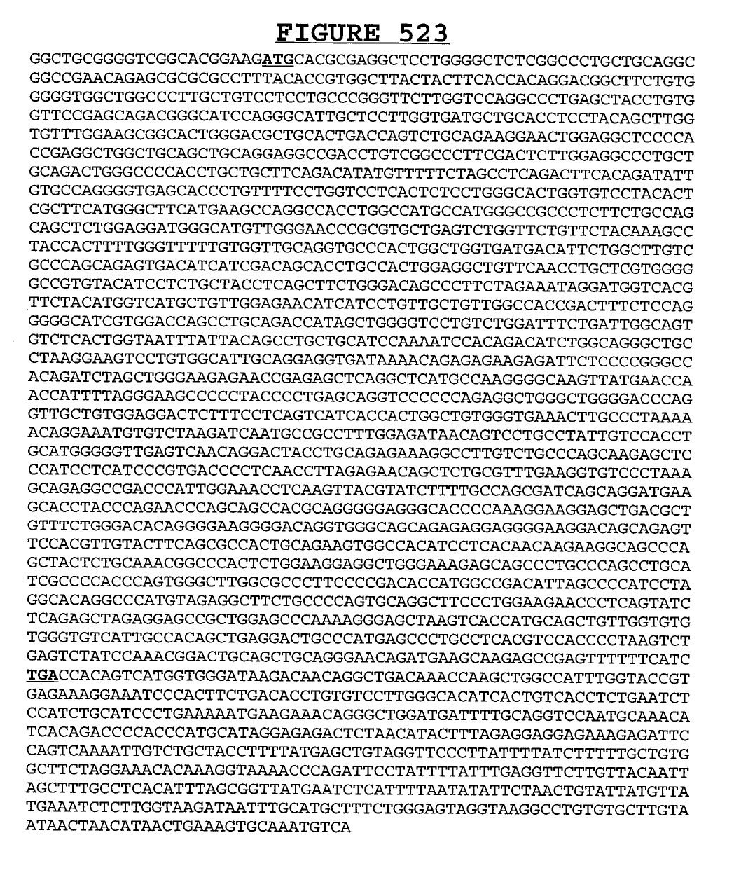

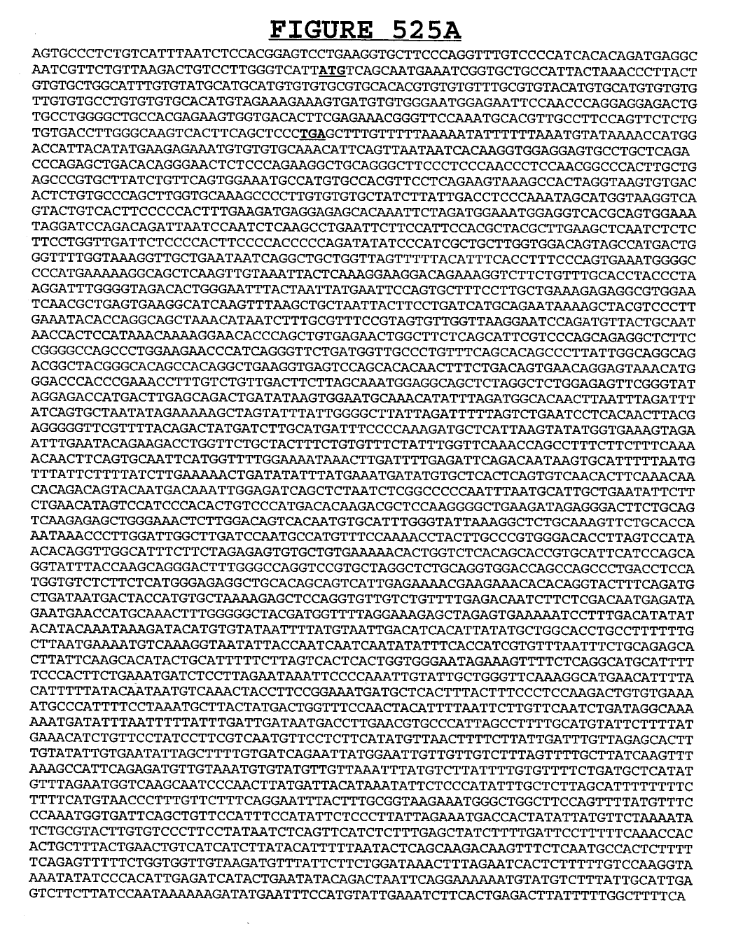

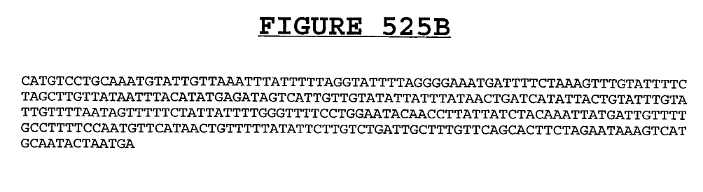

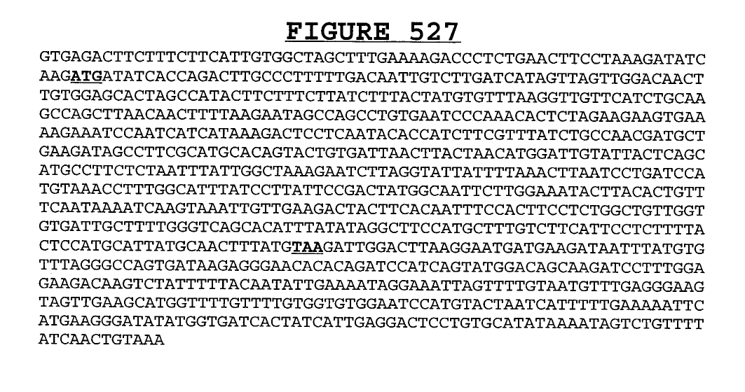

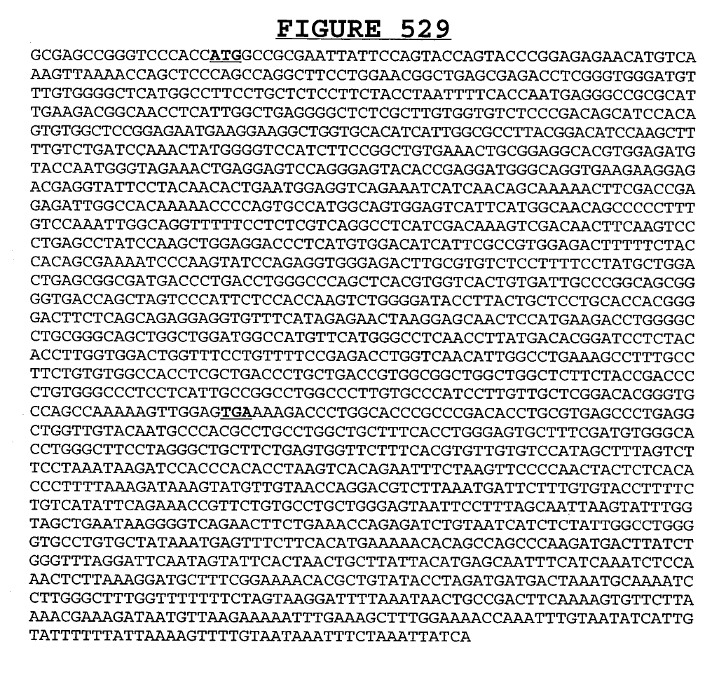

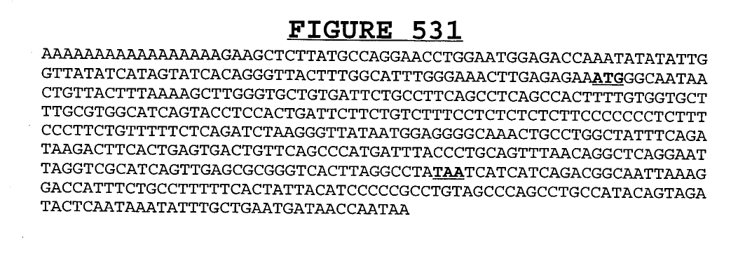

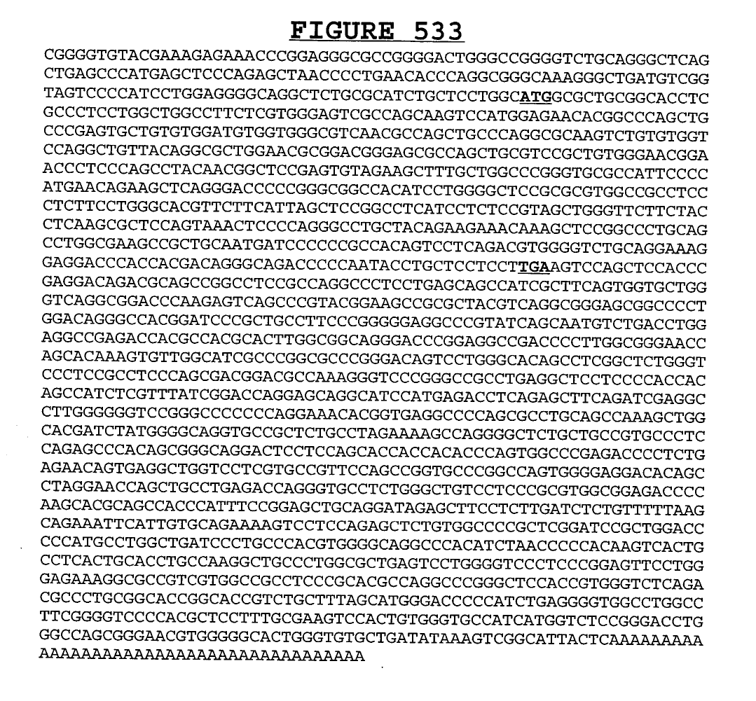

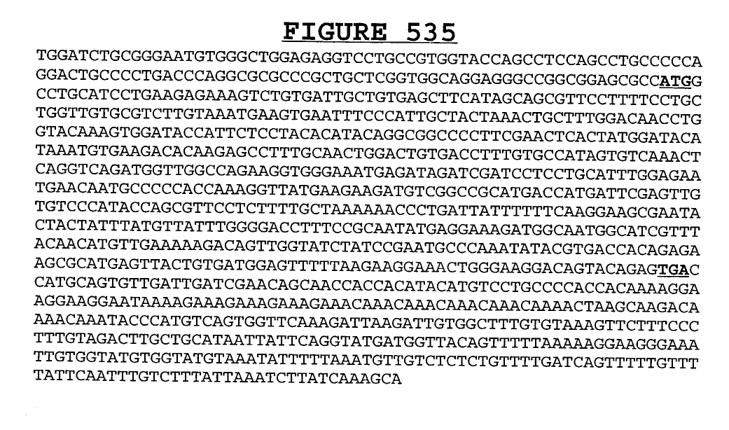

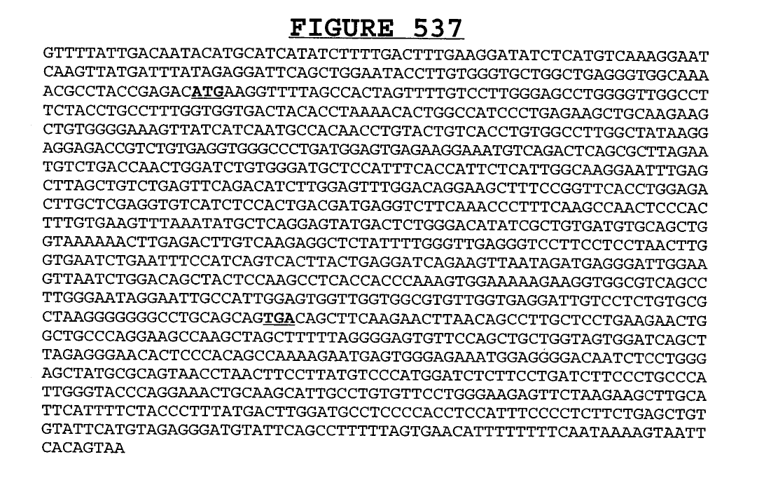

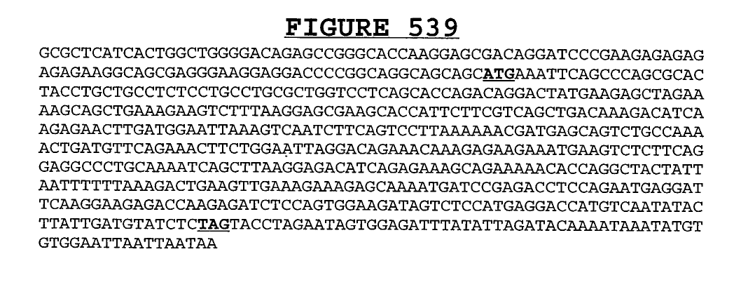

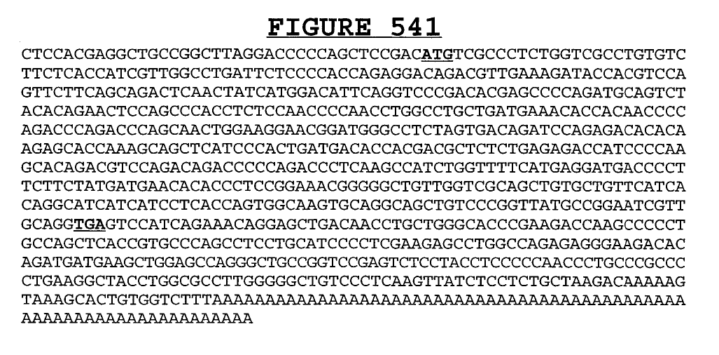

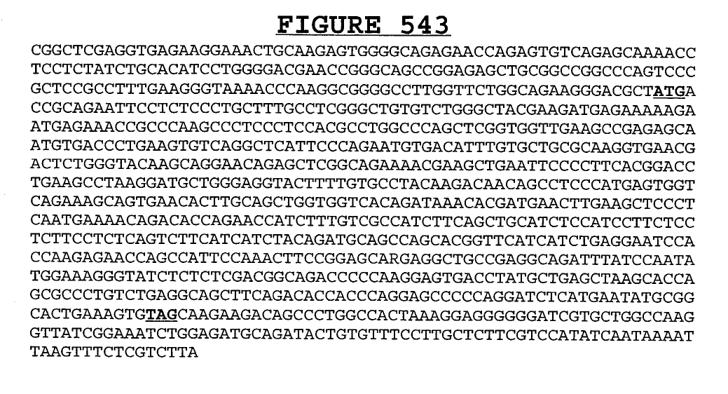

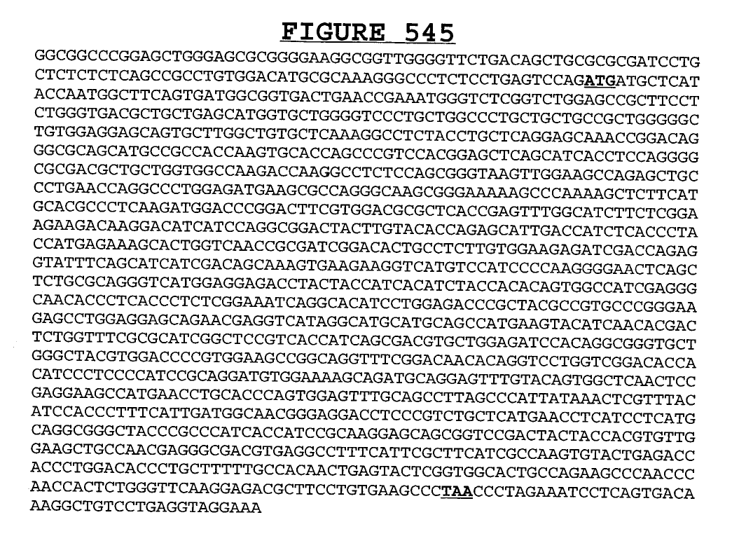

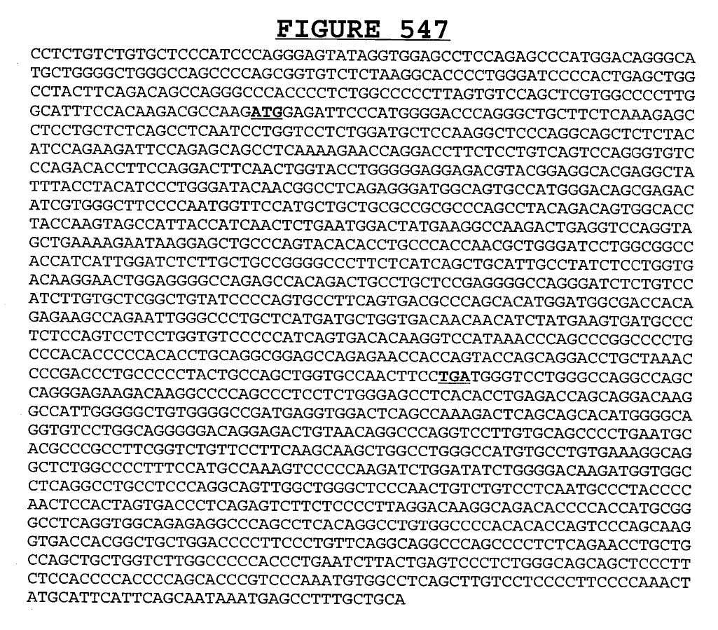

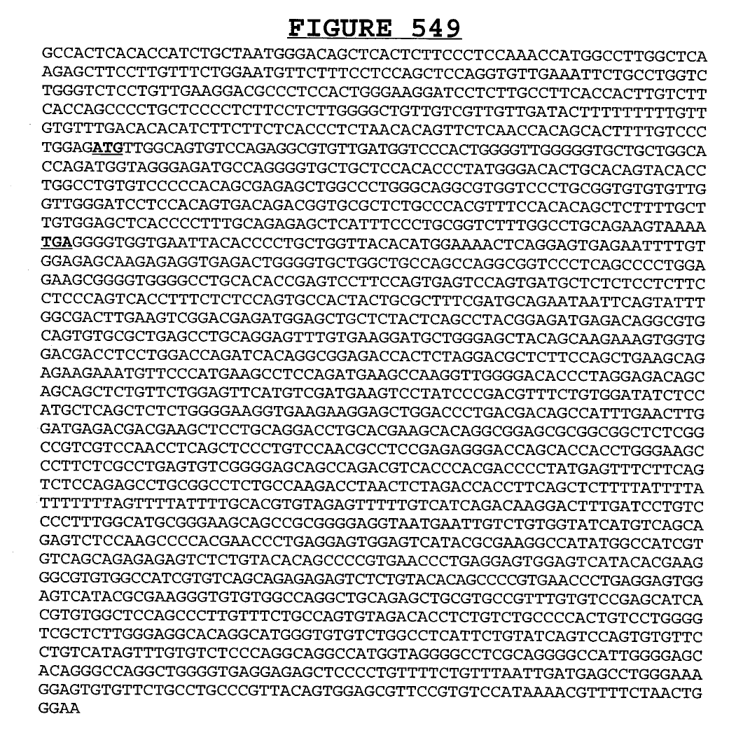

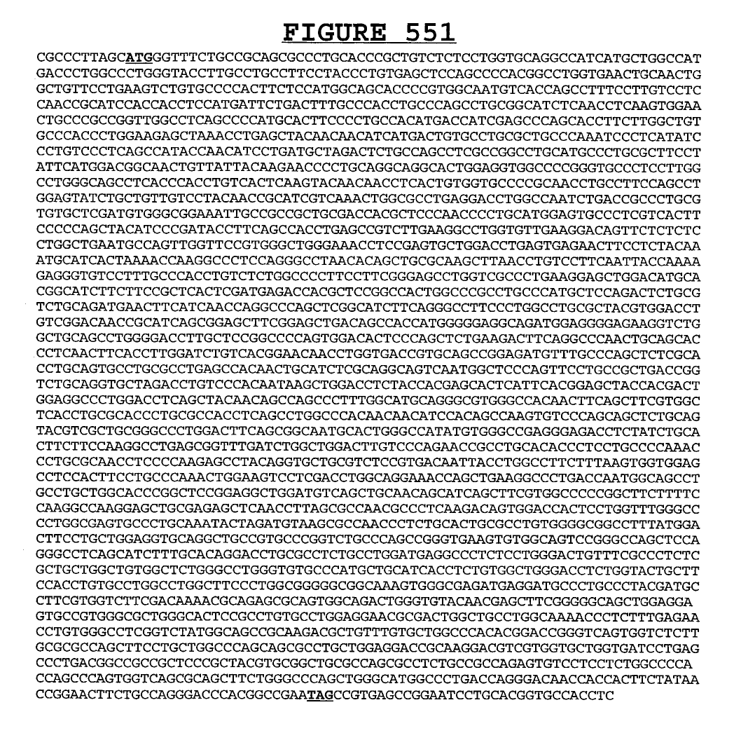

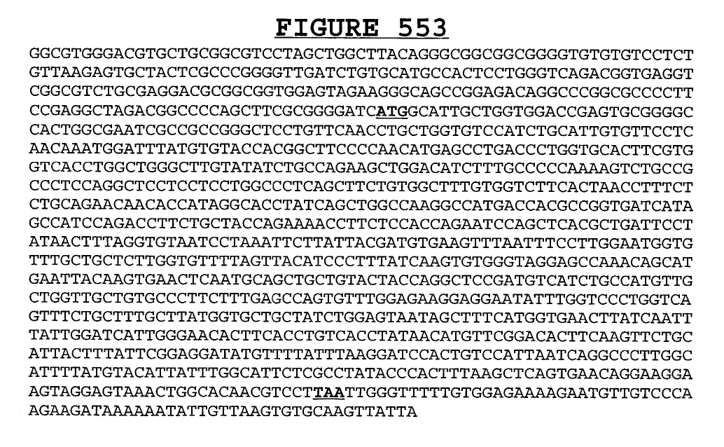

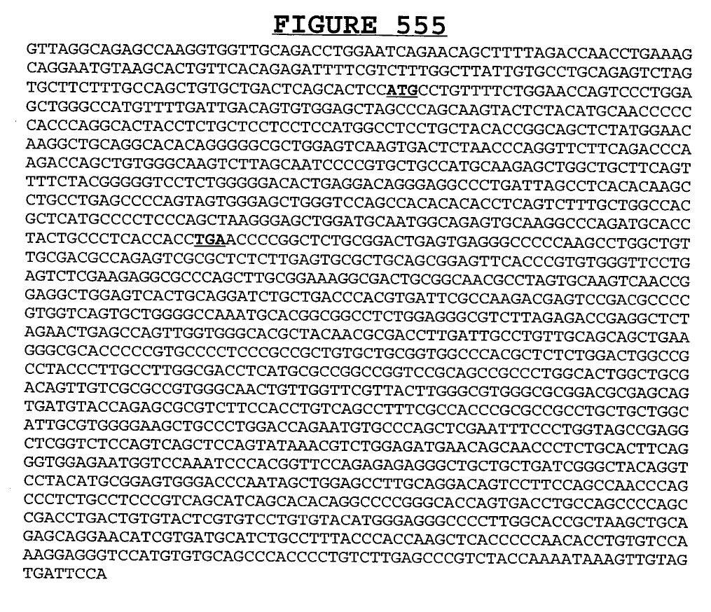

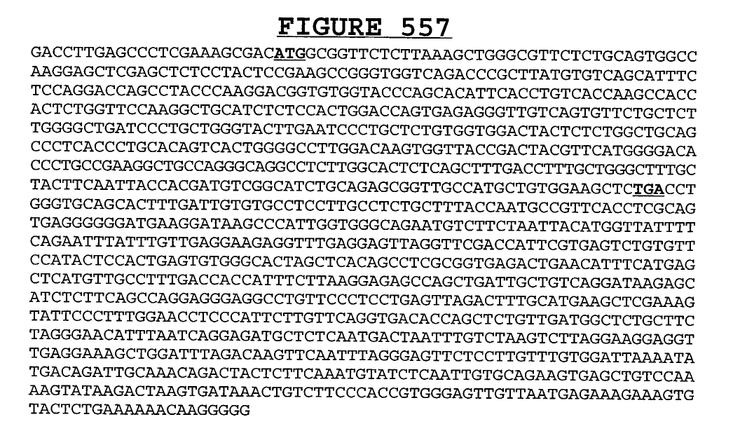

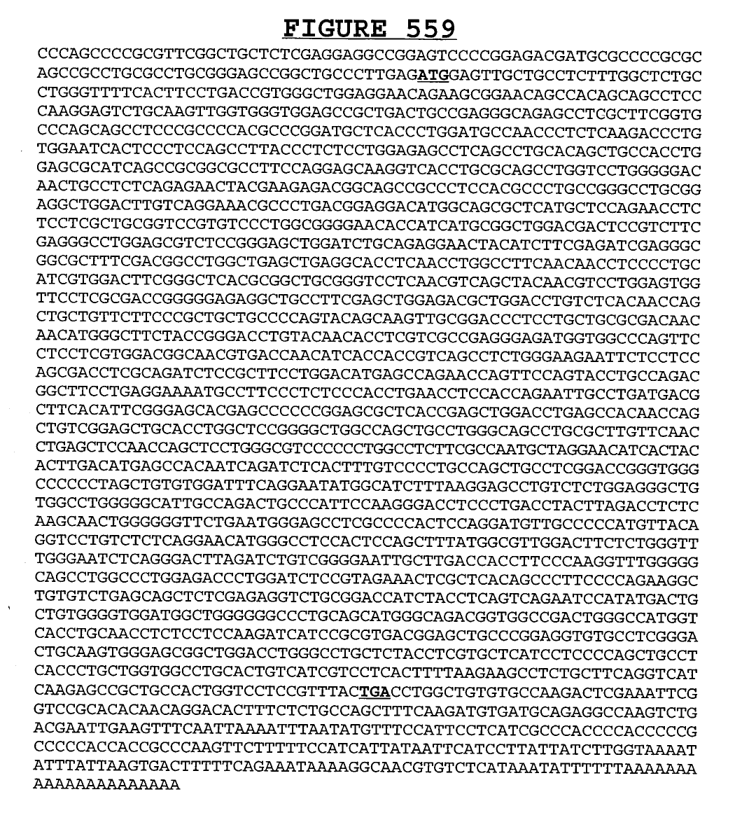

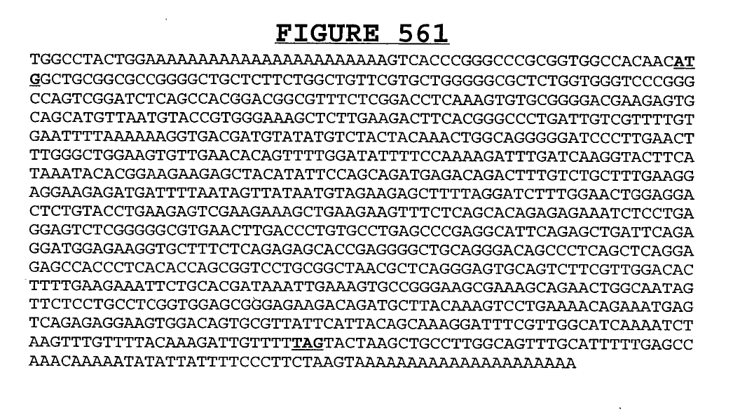

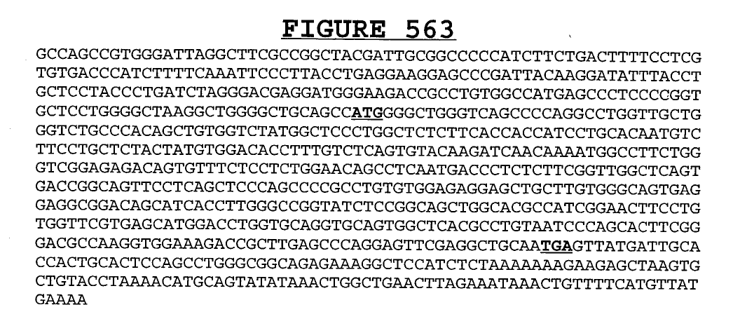

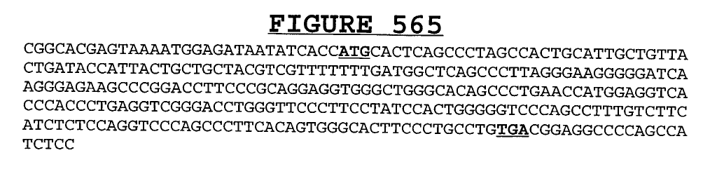

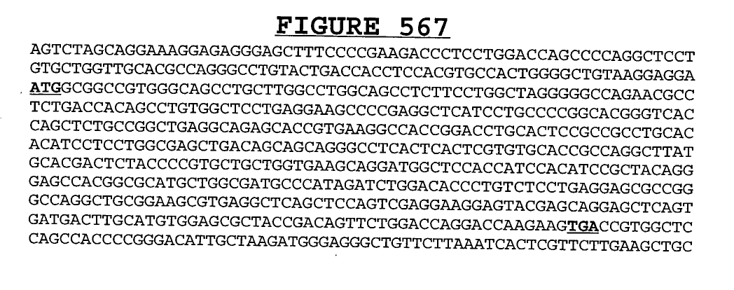

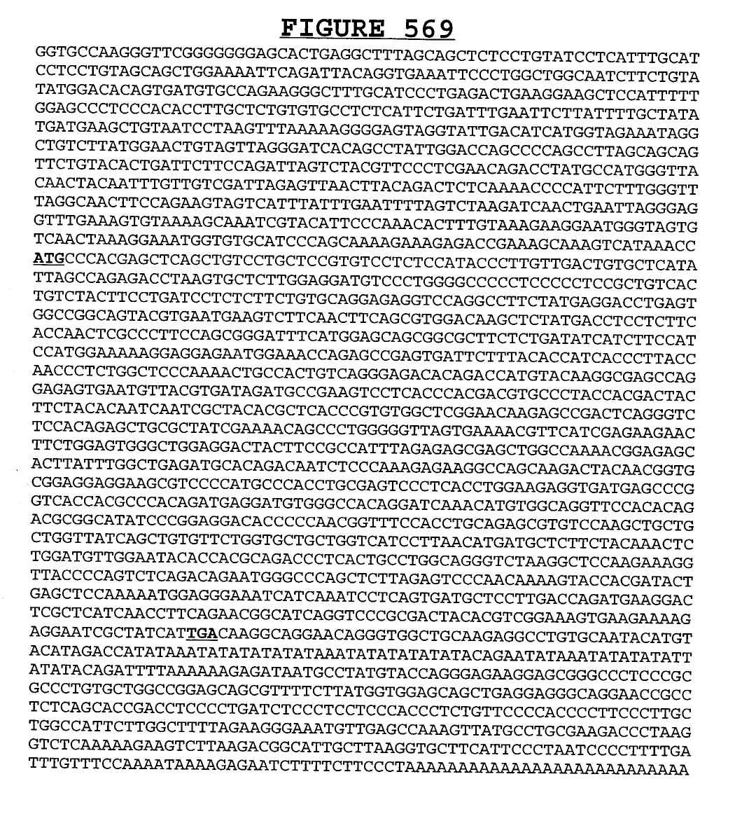

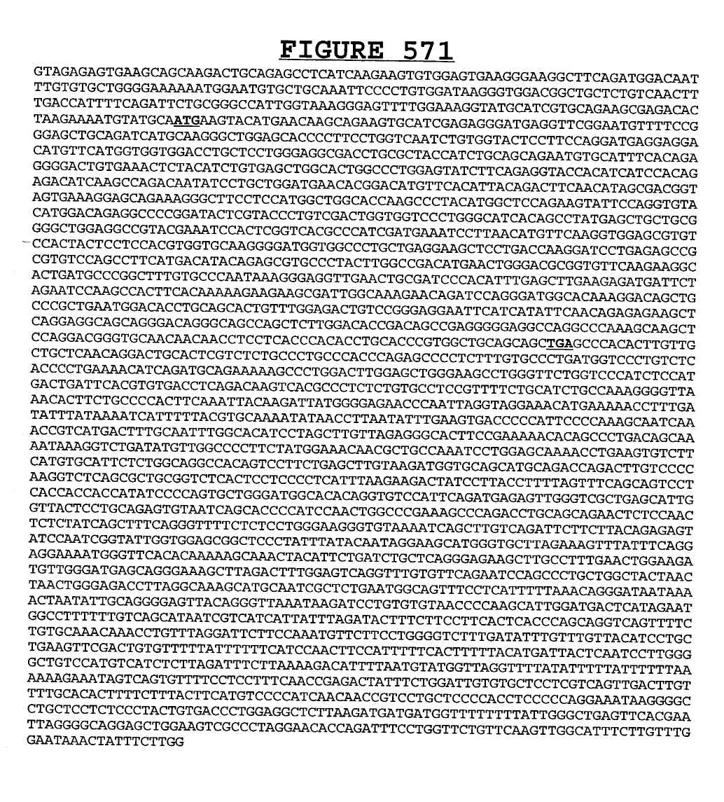

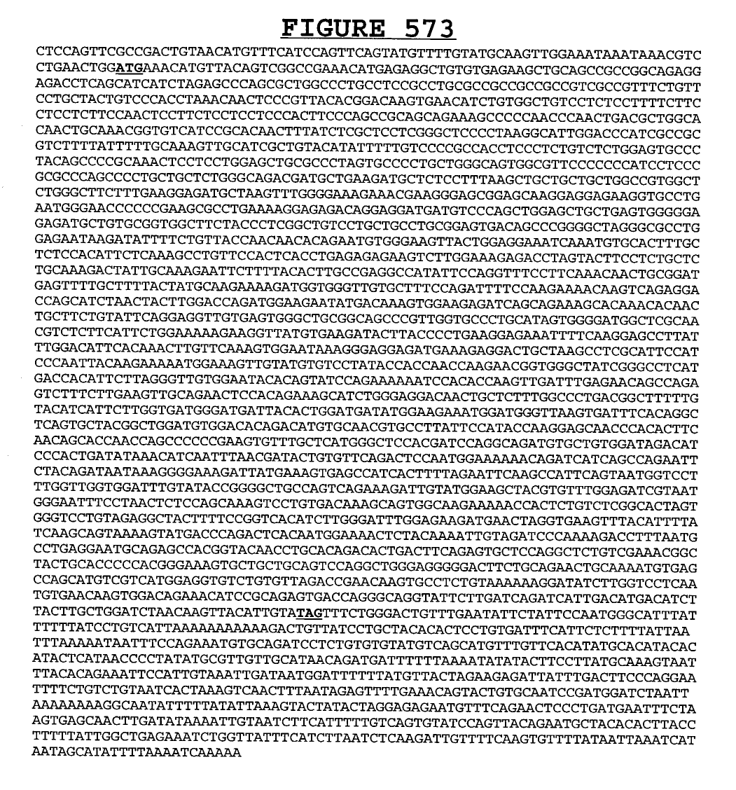

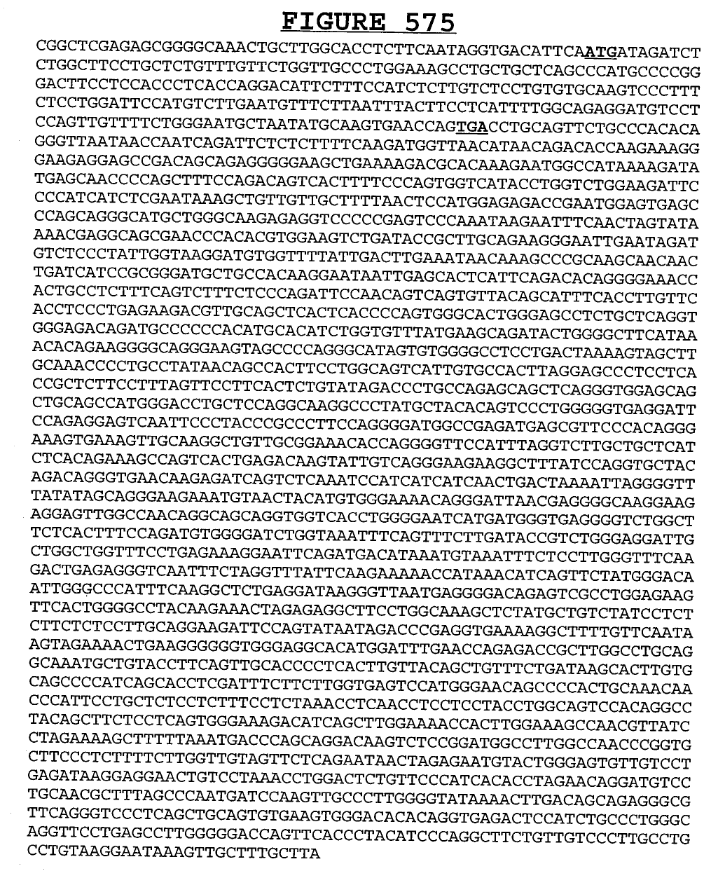

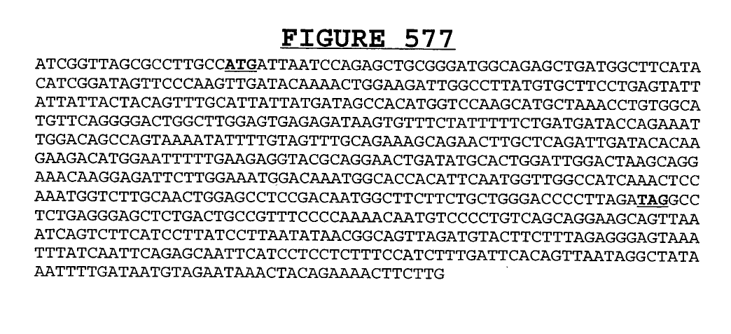

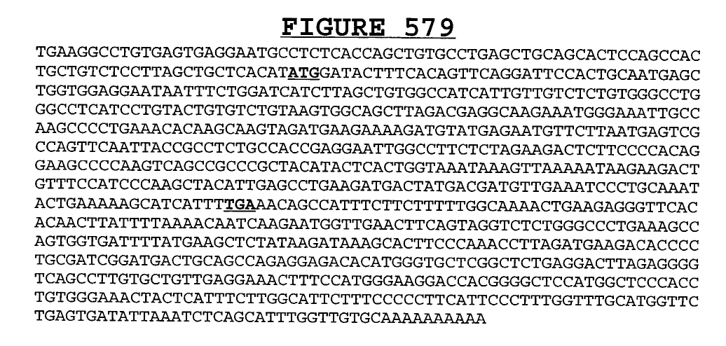

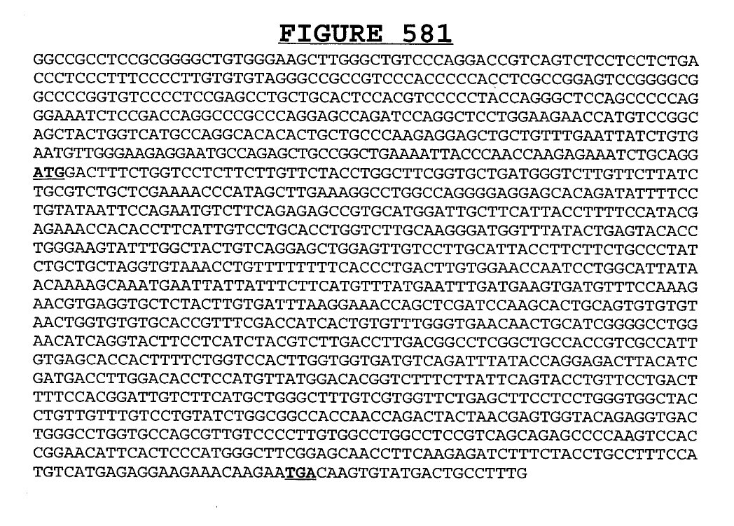

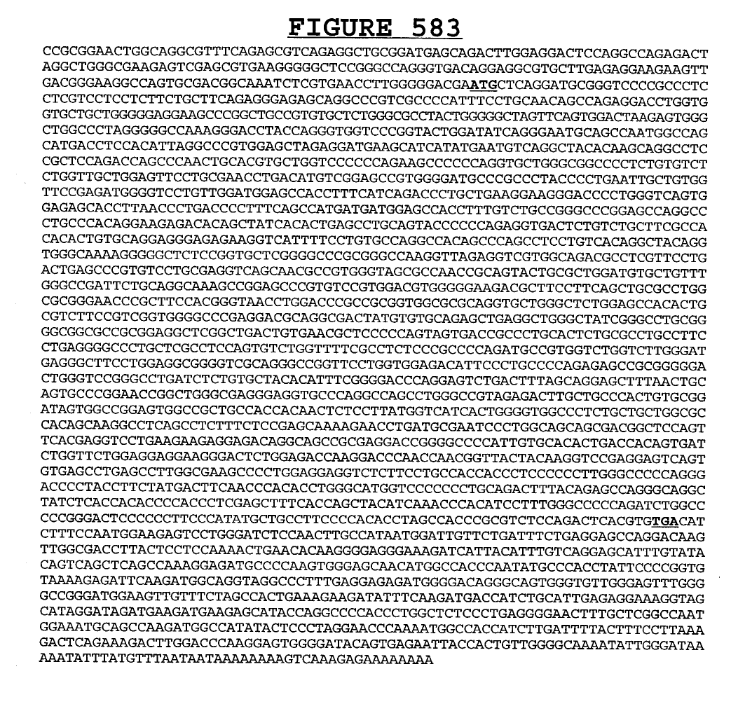

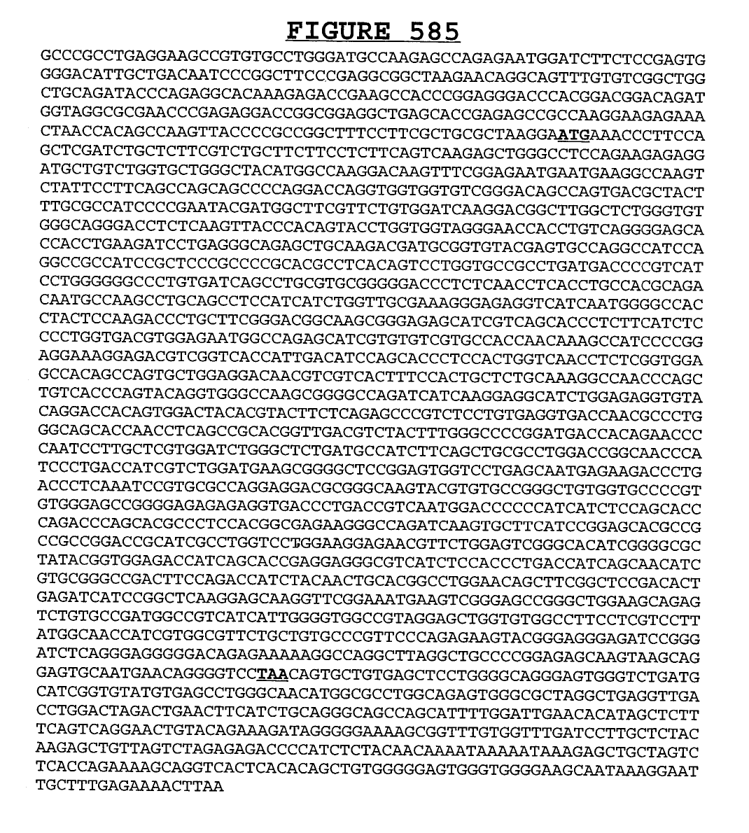

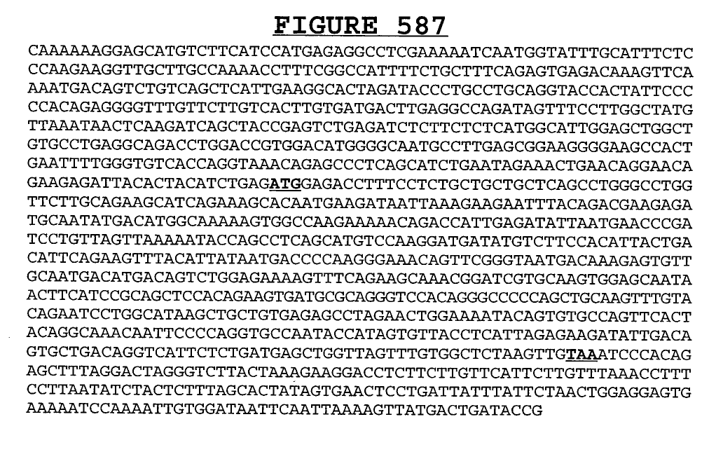

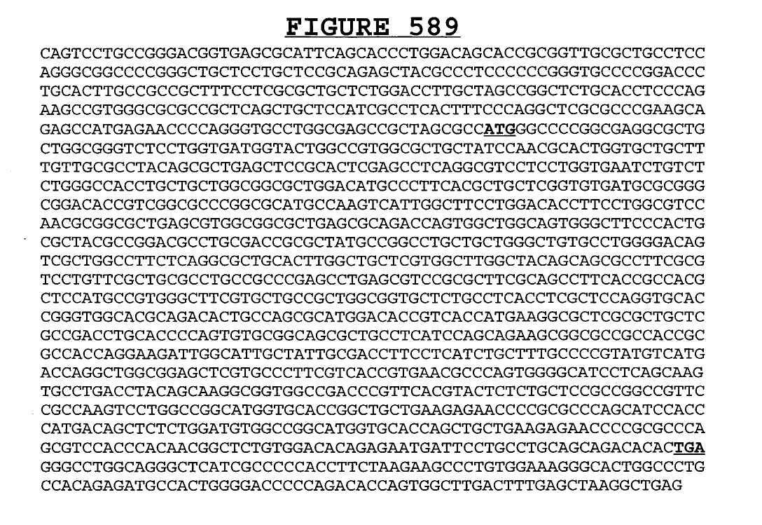

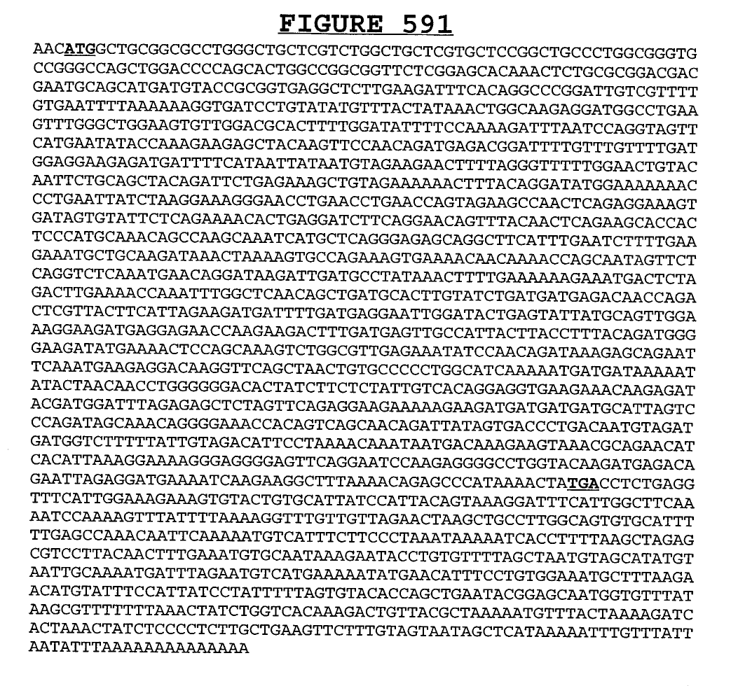

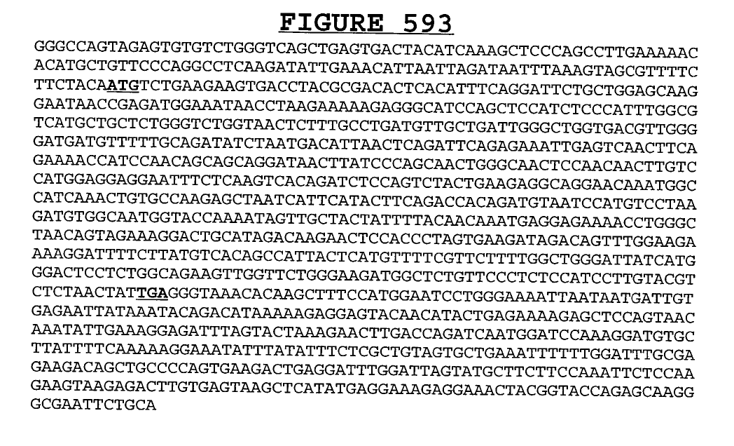

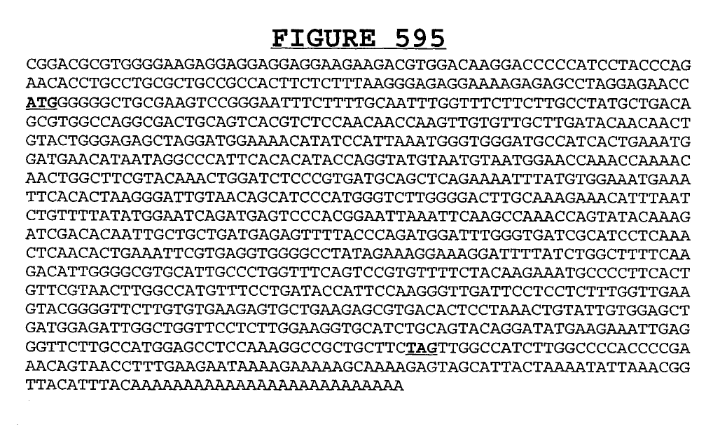

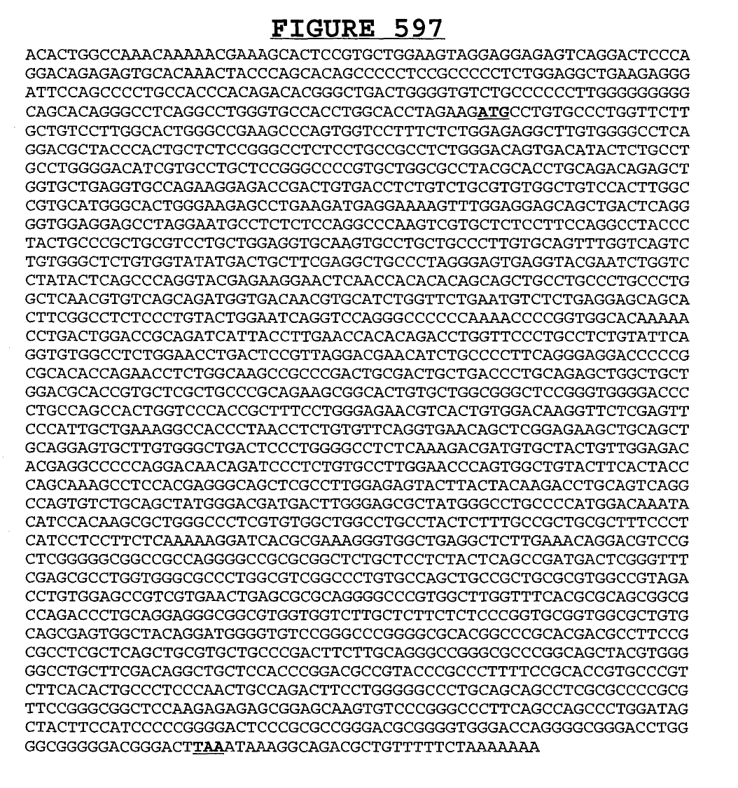

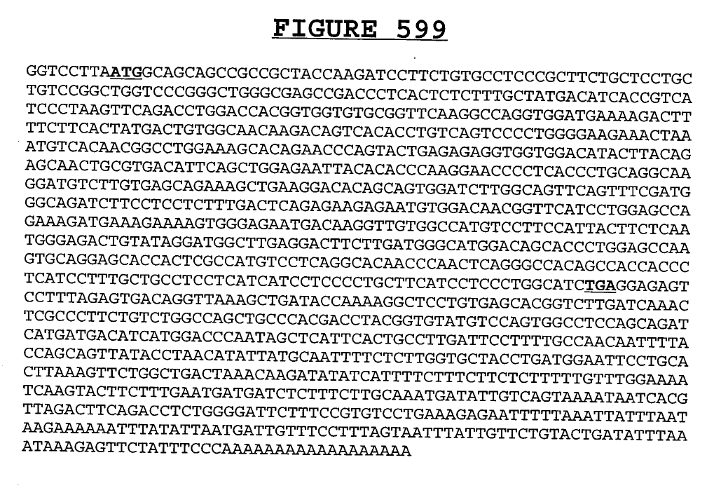

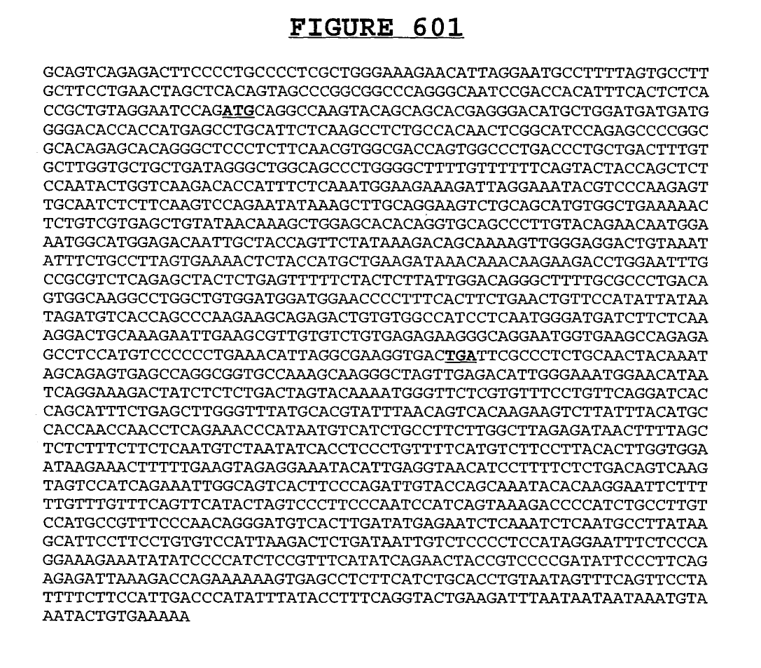

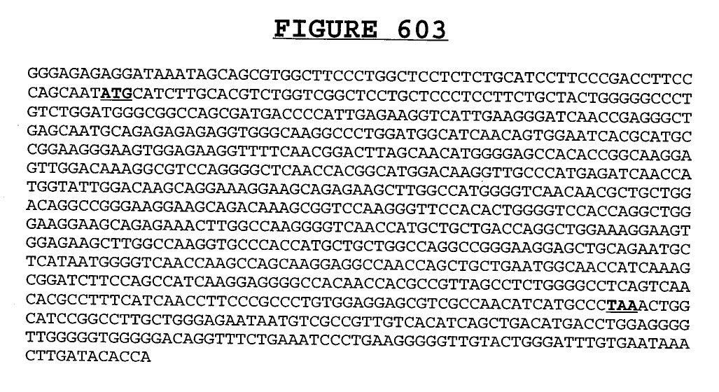

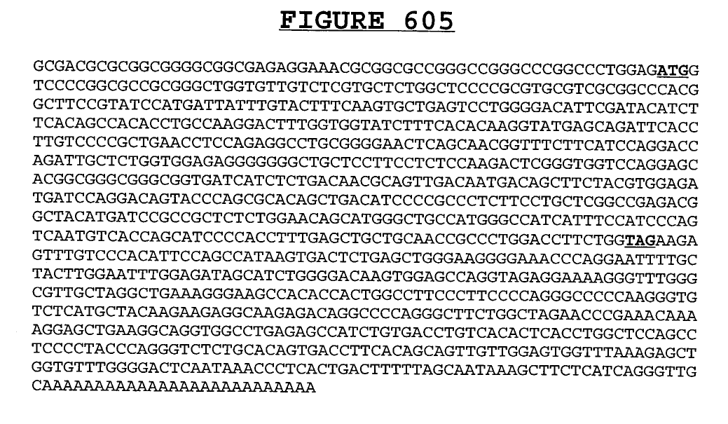

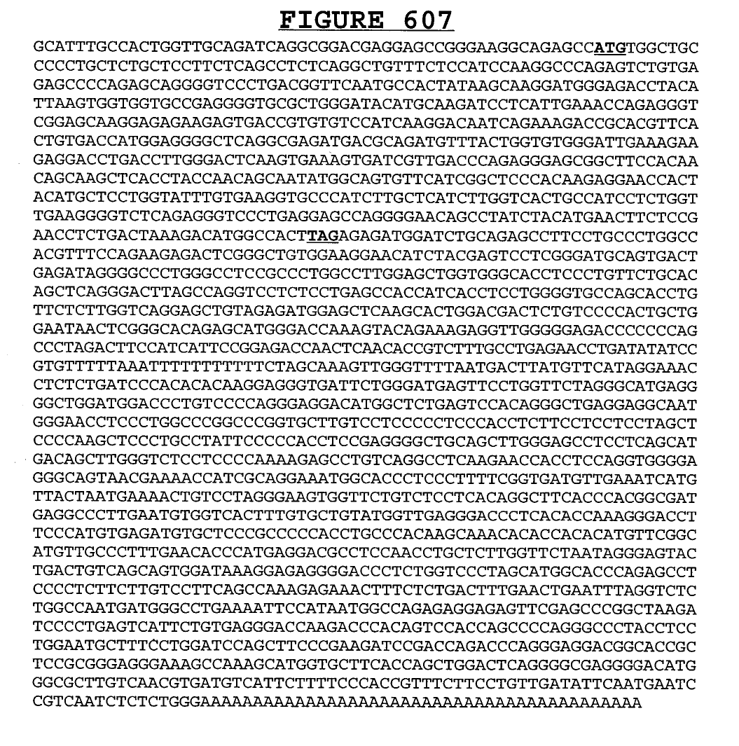

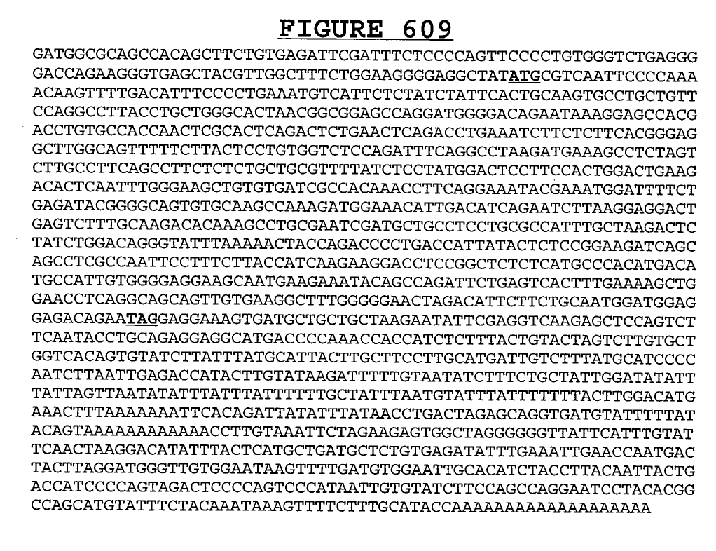

2. Isolated nucleic acid having at least 80% nucleic acid sequence identity to a nucleotide sequence selected from the group consisting of the nucleotide sequence shown in FIG. 1 (SEQ ID NO: 1), FIG. 3 (SEQ ID NO: 3), FIG. 5 (SEQ ID NO: 5), FIG. 7 (SEQ ID NO: 7), FIG. 9 (SEQ ID NO: 9), FIG. 11 (SEQ ID NO: 11), FIG. 13 (SEQ ID NO: 13), FIG. 15 (SEQ ID NO: 15), FIG. 17 (SEQ ID NO: 17), FIG. 19 (SEQ ID NO: 19), FIG. 21 (SEQ ID NO: 21), FIG. 23 (SEQ ID NO: 23), FIG. 25 (SEQ ID NO: 25), FIG. 27 (SEQ ID NO: 27), FIG. 29 (SEQ ID NO: 29), FIG. 31 (SEQ ID NO: 31), FIG. 33 (SEQ ID NO: 33), FIG. 35 (SEQ ID NO: 35), FIG. 37 (SEQ ID NO: 37), FIG. 39 (SEQ ID NO: 39), FIG. 41 (SEQ ID NO: 41), FIG. 43 (SEQ ID NO: 43), FIG. 45 (SEQ ID NO: 45), FIG. 47 (SEQ ID NO: 47), FIG. 49 (SEQ ID NO: 49), FIG. 51 (SEQ ID NO: 51), FIG. 53 (SEQ ID NO: 53), FIG. 55 (SEQ ID NO: 55), FIG. 57 (SEQ ID NO: 57), FIG. 59 (SEQ ID NO: 59), FIG. 61 (SEQ ID NO: 61), FIG. 63 (SEQ ID NO: 63), FIG. 65 (SEQ ID NO: 65), FIG. 67 (SEQ ID NO: 67), FIG. 69 (SEQ ID NO: 69), FIG. 71 (SEQ ID NO: 71), FIG. 73 (SEQ ID NO: 73), FIGS. 75A-75B (SEQ ID NO: 75), FIG. 77 (SEQ ID NO: 77), FIG. 79 (SEQ ID NO: 79), FIG. 81 (SEQ ID NO: 81), FIG. 83 (SEQ ID NO: 83), FIG. 85 (SEQ ID NO: 85), FIG. 87 (SEQ ID NO: 87), FIG. 89 (SEQ ID NO: 89), FIG. 91 (SEQ ID NO: 91), FIG. 93 (SEQ ID NO: 93), FIG. 95 (SEQ ID NO: 95), FIG. 97 (SEQ ID NO: 97), FIG. 99 (SEQ ID NO: 99), FIG. 101 (SEQ ID NO: 101), FIG. 103 (SEQ ID NO: 103), FIG. 105 (SEQ ID NO: 105), FIG. 107 (SEQ ID NO: 107), FIG. 109 (SEQ ID NO: 109), FIG. 111 (SEQ ID NO: 111), FIG. 113 (SEQ ID NO: 113), FIG. 115 (SEQ ID NO: 115), FIG. 117 (SEQ ID NO: 117), FIG. 119 (SEQ ID NO: 119), FIG. 121 (SEQ ID NO: 121), FIG. 123 (SEQ ID NO: 123), FIG. 125 (SEQ ID NO: 125), FIG. 127 (SEQ ID NO: 127), FIG. 129 (SEQ ID NO: 129), FIG. 131 (SEQ ID NO: 131), FIG. 133 (SEQ ID NO: 133), FIG. 135 (SEQ ID NO: 135), FIG. 137 (SEQ ID NO: 137), FIG. 139 (SEQ ID NO: 139), FIG. 141 (SEQ ID NO: 141), FIG. 143 (SEQ ID NO: 143), FIG. 145 (SEQ ID NO: 145), FIG. 147 (SEQ ID NO: 147), FIG. 149 (SEQ ID NO: 149), FIG. 151 (SEQ ID NO: 151), FIG. 153 (SEQ ID NO: 153), FIG. 155 (SEQ ID NO: 155), FIG. 157 (SEQ ID NO: 157), FIG. 159 (SEQ ID NO: 159), FIG. 161 (SEQ ID NO: 161), FIG. 163 (SEQ ID NO: 163), FIG. 165 (SEQ ID NO: 165), FIG. 167 (SEQ ID NO: 167), FIG. 169 (SEQ ID NO: 169), FIG. 171 (SEQ ID NO: 171), FIG. 173 (SEQ ID NO: 173), FIG. 175 (SEQ ID NO: 175), FIG. 177 (SEQ ID NO: 177), FIG. 179 (SEQ ID NO: 179), FIG. 181 (SEQ ID NO: 181), FIG. 183 (SEQ ID NO: 183), FIG. 185 (SEQ ID NO: 185), FIG. 187 (SEQ ID NO: 187), FIG. 189 (SEQ ID NO: 189), FIG. 191 (SEQ ID NO: 191), FIG. 193 (SEQ ID NO: 193), FIG. 195 (SEQ ID NO: 195), FIG. 197 (SEQ ID NO: 197), FIG. 199 (SEQ ID NO: 199), FIG. 201 (SEQ ID NO: 201), FIG. 203 (SEQ ID NO: 203), FIG. 205 (SEQ ID NO: 205), FIG. 207 (SEQ ID NO: 207), FIG. 209 (SEQ ID NO: 209), FIG. 211 (SEQ ID NO: 211), FIG. 213 (SEQ ID NO: 213), FIG. 215 (SEQ ID NO: 215), FIG. 217 (SEQ ID NO: 217), FIG. 219 (SEQ ID NO: 219), FIG. 221 (SEQ ID NO: 221), FIG. 223 (SEQ ID NO: 223), FIG. 225 (SEQ ID NO: 225), FIG. 227 (SEQ ID NO: 227), FIG. 229 (SEQ ID NO: 229), FIG. 231 (SEQ ID NO: 231), FIG. 233 (SEQ ID NO: 233), FIG. 235 (SEQ ID NO: 235), FIG. 237 (SEQ ID NO: 237), FIG. 239 (SEQ ID NO: 239), FIG. 241 (SEQ ID NO: 241), FIG. 243 (SEQ ID NO: 243), FIG. 245 (SEQ ID NO: 245), FIG. 247 (SEQ ID NO: 247), FIG. 249 (SEQ ID NO: 249), FIG. 251 (SEQ ID NO: 251), FIG. 253 (SEQ ID NO: 253), FIG. 255 (SEQ ID NO: 255), FIG. 257 (SEQ ID NO: 257), FIG. 259 (SEQ ID NO: 259), FIG. 261 (SEQ ID NO: 261), FIG. 263 (SEQ ID NO: 263), FIG. 265 (SEQ ID NO: 265), FIG. 267 (SEQ ID NO: 267), FIG. 269 (SEQ ID NO: 269), FIG. 271 (SEQ ID NO: 271), FIG. 273 (SEQ ID NO: 273), FIG. 275 (SEQ ID NO: 275), FIG. 277 (SEQ ID NO: 277), FIG. 279 (SEQ ID NO: 279), FIG. 281 (SEQ ID NO: 281), FIG. 283 (SEQ ID NO: 283), FIG. 285 (SEQ ID NO: 285), FIG. 287 (SEQ ID NO: 287), FIGS. 289A-289B (SEQ ID NO: 289), FIG. 291 (SEQ ID NO: 291), FIG. 293 (SEQ ID NO: 293), FIG. 295 (SEQ ID NO: 295), FIG. 297 (SEQ ID NO: 297), FIG. 299 (SEQ ID NO: 299), FIG. 301 (SEQ ID NO: 301), FIG. 303 (SEQ ID NO: 303), FIG. 305 (SEQ ID NO: 305), FIG. 307 (SEQ ID NO: 307), FIG. 309 (SEQ ID NO: 309), FIGS. 311A-311B (SEQ ID NO: 311), FIG. 313 (SEQ ID NO: 313), FIG. 315 (SEQ ID NO: 315), FIG. 317 (SEQ ID NO: 317), FIG. 319 (SEQ ID NO: 319), FIG. 321 (SEQ ID NO: 321), FIG. 323 (SEQ ID NO: 323), FIG. 325 (SEQ ID NO: 325), FIG. 327 (SEQ ID NO: 327), FIG. 329 (SEQ ID NO: 329), FIG. 331 (SEQ ID NO: 331), FIG. 333 (SEQ ID NO: 333), FIG. 335 (SEQ ID NO: 335), FIG. 337 (SEQ ID NO: 337), FIG. 339 (SEQ ID NO: 339), FIG. 341 (SEQ ID NO: 341), FIG. 343 (SEQ ID NO: 343), FIG. 345 (SEQ ID NO: 345), FIG. 347 (SEQ ID NO: 347), FIG. 349 (SEQ ID NO: 349), FIGS. 351A-351B (SEQ ID NO: 351), FIG. 353 (SEQ ID NO: 353), FIG. 355 (SEQ ID NO: 355), FIG. 357 (SEQ ID NO: 357), FIG. 359 (SEQ ID NO: 359), FIG. 361 (SEQ ID NO: 361), FIG. 363 (SEQ ID NO: 363), FIG. 365 (SEQ ID NO: 365), FIG. 367 (SEQ ID NO: 367), FIG. 369 (SEQ ID NO: 369), FIG. 371 (SEQ ID NO: 371), FIG. 373 (SEQ ID NO: 373), FIG. 375 (SEQ ID NO: 375), FIG. 377 (SEQ ID NO: 377), FIG. 379 (SEQ ID NO: 379), FIG. 381 (SEQ ID NO: 38 1), FIG. 383 (SEQ ID NO: 383), FIG. 385 (SEQ ID NO: 385), FIG. 387 (SEQ ID NO: 387), FIG. 389 (SEQ ID NO: 389), FIG. 391 (SEQ ID NO: 391), FIG. 393 (SEQ ID NO: 393), FIG. 395 (SEQ ID NO: 395), FIG. 397 (SEQ ID NO: 397), FIG. 399 (SEQ ID NO: 399), FIG. 401 (SEQ ID NO: 401), FIG. 403 (SEQ ID NO: 403), FIG. 405 (SEQ ID NO: 405), FIG. 407 (SEQ ID NO: 407), FIG. 409 (SEQ ID NO: 409), FIG. 411 (SEQ ID NO: 411), FIG. 413 (SEQ ID NO: 413), FIG. 415 (SEQ ID NO: 415), FIG. 417 (SEQ ID NO: 417), FIG. 419 (SEQ ID NO: 419), FIG. 421 (SEQ ID NO: 421), FIG. 423 (SEQ ID NO: 423), FIG. 425 (SEQ ID NO: 425), FIG. 427 (SEQ ID NO: 427), FIG. 429 (SEQ ID NO: 429), FIG. 431 (SEQ ID NO: 431), FIG. 433 (SEQ ID NO: 433), FIG. 435 (SEQ ID NO: 435), FIG. 437 (SEQ ID NO: 437), FIG. 439 (SEQ ID NO: 439), FIG. 441 (SEQ ID NO: 441), FIG. 443 (SEQ ID NO: 443), FIG. 445 (SEQ ID NO: 445), FIG. 447 (SEQ ID NO: 447), FIG. 449 (SEQ ID NO: 449), FIG. 451 (SEQ ID NO: 451), FIG. 453 (SEQ ID NO: 453), FIG. 455 (SEQ ID NO: 455), FIG. 457 (SEQ ID NO: 457), FIG. 459 (SEQ ID NO: 459), FIG. 461 (SEQ ID NO: 461), FIG. 463 (SEQ ID NO: 463), FIG. 465 (SEQ ID NO: 465), FIG. 467 (SEQ ID NO: 467), FIG. 469 (SEQ ID NO: 469), FIG. 471 (SEQ ID NO: 471), FIG. 473 (SEQ ID NO: 473), FIG. 475 (SEQ ID NO: 475), FIG. 477 (SEQ ID NO: 477), FIG. 479 (SEQ ID NO: 479), FIG. 481 (SEQ ID NO: 481), FIG. 483 (SEQ ID NO: 483), FIG. 485 (SEQ ID NO: 485), FIG. 487 (SEQ ID NO: 487), FIG. 489 (SEQ ID NO: 489), FIG. 491 (SEQ ID NO: 491), FIG. 493 (SEQ ID NO: 493), FIG. 495 (SEQ ID NO: 495), FIG. 497 (SEQ ID NO: 497), FIG. 499 (SEQ ID NO: 499), FIG. 501 (SEQ ID NO: 501), FIG. 503 (SEQ ID NO: 503), FIG. 505 (SEQ ID NO: 505), FIG. 507 (SEQ ID NO: 507), FIG. 509 (SEQ ID NO: 509), FIG. 511 (SEQ ID NO: 511), FIG. 513 (SEQ ID NO: 513), FIG. 515 (SEQ ID NO: 515), FIG. 517 (SEQ ID NO: 517), FIG. 519 (SEQ ID NO: 519), FIG. 521 (SEQ ID NO: 521), FIG. 523 (SEQ ID NO: 523), FIGS. 525A-525B (SEQ ID NO: 525), FIG. 527 (SEQ ID NO: 527), FIG. 529 (SEQ ID NO: 529), FIG. 531 (SEQ ID NO: 531), FIG. 533 (SEQ ID NO: 533), FIG. 535 (SEQ ID NO: 535), FIG. 537 (SEQ ID NO: 537), FIG. 539 (SEQ ID NO: 539), FIG. 541 (SEQ ID NO: 541), FIG. 543 (SEQ ID NO: 543), FIG. 545 (SEQ ID NO: 545), FIG. 547 (SEQ ID NO: 547), FIG. 549 (SEQ ID NO: 549), FIG. 551 (SEQ ID NO: 551), FIG. 553 (SEQ ID NO: 553), FIG. 555 (SEQ ID NO: 555), FIG. 557 (SEQ ID NO: 557), FIG. 559 (SEQ ID NO: 559), FIG. 561 (SEQ ID NO: 561), FIG. 563 (SEQ ID NO: 563), FIG. 565 (SEQ ID NO: 565), FIG. 567 (SEQ ID NO: 567), FIG. 569 (SEQ ID NO: 569), FIG. 571 (SEQ ID NO: 571), FIG. 573 (SEQ ID NO: 573), FIG. 575 (SEQ ID NO: 575), FIG. 577 (SEQ ID NO: 577), FIG. 579 (SEQ ID NO: 579), FIG. 581 (SEQ ID NO: 581), FIG. 583 (SEQ ID NO: 583), FIG. 585 (SEQ ID NO: 585), FIG. 587 (SEQ ID NO: 587), FIG. 589 (SEQ ID NO: 589), FIG. 591 (SEQ ID NO: 591), FIG. 593 (SEQ ID NO: 593), FIG. 595 (SEQ ID NO: 595), FIG. 597 (SEQ ID NO: 597), FIG. 599 (SEQ ID NO: 599), FIG. 601 (SEQ ID NO: 601), FIG. 603 (SEQ ID NO: 603), FIG. 605 (SEQ ID NO: 605), FIG. 607 (SEQ ID NO: 607), and FIG. 609 (SEQ ID NO: 609).

3. Isolated nucleic acid having at least 80% nucleic acid sequence identity to a nucleotide sequence selected from the group consisting of the full-length coding sequence of the nucleotide sequence shown in FIG. 1 (SEQ ID NO: 1), FIG. 3 (SEQ ID NO: 3), FIG. 5 (SEQ ID NO: 5), FIG. 7 (SEQ ID NO: 7), FIG. 9 (SEQ ID NO: 9), FIG. 11 (SEQ ID NO: 11), FIG. 13 (SEQ ID NO: 13), FIG. 15 (SEQ ID NO: 15), FIG. 17 (SEQ ID NO: 17), FIG. 19 (SEQ ID NO: 19), FIG. 21 (SEQ ID NO: 21), FIG. 23 (SEQ ID NO: 23), FIG. 25 (SEQ ID NO: 25), FIG. 27 (SEQ ID NO: 27), FIG. 29 (SEQ ID NO: 29), FIG. 31 (SEQ ID NO: 31), FIG. 33 (SEQ ID NO: 33), FIG. 35 (SEQ ID NO: 35), FIG. 37 (SEQ ID NO: 37), FIG. 39 (SEQ ID NO: 39), FIG. 41 (SEQ ID NO: 41), FIG. 43 (SEQ ID NO: 43), FIG. 45 (SEQ ID NO: 45), FIG. 47 (SEQ ID NO: 47), FIG. 49 (SEQ ID NO: 49), FIG. 51 (SEQ ID NO: 51), FIG. 53 (SEQ ID NO: 53), FIG. 55 (SEQ ID NO: 55), FIG. 57 (SEQ ID NO: 57), FIG. 59 (SEQ ID NO: 59), FIG. 61 (SEQ ID NO: 61), FIG. 63 (SEQ ID NO: 63), FIG. 65 (SEQ ID NO: 65), FIG. 67 (SEQ ID NO: 67), FIG. 69 (SEQ ID NO: 69), FIG. 71 (SEQ ID NO: 71), FIG. 73 (SEQ ID NO: 73), FIGS. 75A-75B (SEQ ID NO: 75), FIG. 77 (SEQ ID NO: 77), FIG. 79 (SEQ ID NO: 79), FIG. 81 (SEQ ID NO: 81), FIG. 83 (SEQ ID NO: 83), FIG. 85 (SEQ ID NO: 85), FIG. 87 (SEQ ID NO: 87), FIG. 89 (SEQ ID NO: 89), FIG. 91 (SEQ ID NO: 91), FIG. 93 (SEQ ID NO: 93), FIG. 95 (SEQ ID NO: 95), FIG. 97 (SEQ ID NO: 97), FIG. 99 (SEQ ID NO: 99), FIG. 101 (SEQ ID NO: 101), FIG. 103 (SEQ ID NO: 103), FIG. 105 (SEQ ID NO: 105), FIG. 107 (SEQ ID NO: 107), FIG. 109 (SEQ ID NO: 109), FIG. 111 (SEQ ID NO: 111), FIG. 113 (SEQ ID NO: 113), FIG. 115 (SEQ ID NO: 115), FIG. 117 (SEQ ID NO: 117), FIG. 119 (SEQ ID NO: 119), FIG. 121 (SEQ ID NO: 121), FIG. 123 (SEQ ID NO: 123), FIG. 125 (SEQ ID NO: 125), FIG. 127 (SEQ ID NO: 127), FIG. 129 (SEQ ID NO: 129), FIG. 131 (SEQ ID NO: 131), FIG. 133 (SEQ ID NO: 133), FIG. 135 (SEQ ID NO: 135), FIG. 137 (SEQ ID NO: 137), FIG. 139 (SEQ ID NO: 139), FIG. 141 (SEQ ID NO: 141), FIG. 143 (SEQ ID NO: 143), FIG. 145 (SEQ ID NO: 145), FIG. 147 (SEQ ID NO: 147), FIG. 149 (SEQ ID NO: 149), FIG. 151 (SEQ ID NO: 151), FIG. 153 (SEQ ID NO: 153), FIG. 155 (SEQ ID NO: 155), FIG. 157 (SEQ ID NO: 157), FIG. 159 (SEQ ID NO: 159), FIG. 161 (SEQ ID NO: 161), FIG. 163 (SEQ ID NO: 163), FIG. 165 (SEQ ID NO: 165), FIG. 167 (SEQ ID NO: 167), FIG. 169 (SEQ ID NO: 169), FIG. 171 (SEQ ID NO: 171), FIG. 173 (SEQ ID NO: 173), FIG. 175 (SEQ ID NO: 175), FIG. 177 (SEQ ID NO: 177), FIG. 179 (SEQ ID NO: 179), FIG. 181 (SEQ ID NO: 181), FIG. 183 (SEQ ID NO: 183), FIG. 185 (SEQ ID NO: 185), FIG. 187 (SEQ ID NO: 187), FIG. 189 (SEQ ID NO: 189), FIG. 191 (SEQ ID NO: 191), FIG. 193 (SEQ ID NO: 193), FIG. 195 (SEQ ID NO: 195), FIG. 197 (SEQ ID NO: 197), FIG. 199 (SEQ ID NO: 199), FIG. 201 (SEQ ID NO: 201), FIG. 203 (SEQ ID NO: 203), FIG. 205 (SEQ ID NO: 205), FIG. 207 (SEQ ID NO: 207), FIG. 209 (SEQ ID NO: 209), FIG. 211 (SEQ ID NO: 211), FIG. 213 (SEQ ID NO: 213), FIG. 215 (SEQ ID NO: 215), FIG. 217 (SEQ ID NO: 217), FIG. 219 (SEQ ID NO: 219), FIG. 221 (SEQ ID NO: 221), FIG. 223 (SEQ ID NO: 223), FIG. 225 (SEQ ID NO: 225), FIG. 227 (SEQ ID NO: 227), FIG. 229 (SEQ ID NO: 229), FIG. 231 (SEQ ID NO: 231), FIG. 233 (SEQ ID NO: 233), FIG. 235 (SEQ ID NO: 235), FIG. 237 (SEQ ID NO: 237), FIG. 239 (SEQ ID NO: 239), FIG. 241 (SEQ ID NO: 241), FIG. 243 (SEQ ID NO: 243), FIG. 245 (SEQ ID NO: 245), FIG. 247 (SEQ ID NO: 247), FIG. 249 (SEQ ID NO: 249), FIG. 251 (SEQ ID NO: 25 1), FIG. 253 (SEQ ID NO: 253), FIG. 255 (SEQ ID NO: 255), FIG. 257 (SEQ ID NO: 257), FIG. 259 (SEQ ID NO: 259), FIG. 261 (SEQ ID NO: 261), FIG. 263 (SEQ ID NO: 263), FIG. 265 (SEQ ID NO: 265), FIG. 267 (SEQ ID NO: 267), FIG. 269 (SEQ ID NO: 269), FIG. 271 (SEQ ID NO: 271), FIG. 273 (SEQ ID NO: 273), FIG. 275 (SEQ ID NO: 275), FIG. 277 (SEQ ID NO: 277), FIG. 279 (SEQ ID NO: 279), FIG. 281 (SEQ ID NO: 281), FIG. 283 (SEQ ID NO: 283), FIG. 285 (SEQ ID NO: 285), FIG. 287 (SEQ ID NO: 287), FIGS. 289A-289B (SEQ ID NO: 289), FIG. 291 (SEQ ID NO: 291), FIG. 293 (SEQ ID NO: 293), FIG. 295 (SEQ ID NO: 295), FIG. 297 (SEQ ID NO: 297), FIG. 299 (SEQ ID NO: 299), FIG. 301 (SEQ ID NO: 301), FIG. 303 (SEQ ID NO: 303), FIG. 305 (SEQ ID NO: 305), FIG. 307 (SEQ ID NO: 307), FIG. 309 (SEQ ID NO: 309), FIGS. 311A-311B (SEQ ID NO: 311), FIG. 313 (SEQ ID NO: 313), FIG. 315 (SEQ ID NO: 315), FIG. 317 (SEQ ID NO: 317), FIG. 319 (SEQ ID NO: 319), FIG. 321 (SEQ ID NO: 321), FIG. 323 (SEQ ID NO: 323), FIG. 325 (SEQ ID NO: 325), FIG. 327 (SEQ ID NO: 327), FIG. 329 (SEQ ID NO: 329), FIG. 331 (SEQ ID NO: 331), FIG. 333 (SEQ ID NO: 333), FIG. 335 (SEQ ID NO: 335), FIG. 337 (SEQ ID NO: 337), FIG. 339 (SEQ ID NO: 339), FIG. 341 (SEQ ID NO: 341), FIG. 343 (SEQ ID NO: 343), FIG. 345 (SEQ ID NO: 345), FIG. 347 (SEQ ID NO: 347), FIG. 349 (SEQ ID NO: 349), FIGS. 351A-351B (SEQ ID NO: 351), FIG. 353 (SEQ ID NO: 353), FIG. 355 (SEQ ID NO: 355), FIG. 357 (SEQ ID NO: 357), FIG. 359 (SEQ ID NO: 359), FIG. 361 (SEQ ID NO: 361), FIG. 363 (SEQ ID NO: 363), FIG. 365 (SEQ ID NO: 365), FIG. 367 (SEQ ID NO: 367), FIG. 369 (SEQ ID NO: 369), FIG. 371 (SEQ ID NO: 371), FIG. 373 (SEQ ID NO: 373), FIG. 375 (SEQ ID NO: 375), FIG. 377 (SEQ ID NO: 377), FIG. 379 (SEQ ID NO: 379), FIG. 381 (SEQ ID NO: 381), FIG. 383 (SEQ ID NO: 383), FIG. 385 (SEQ ID NO: 385), FIG. 387 (SEQ ID NO: 387), FIG. 389 (SEQ ID NO: 389), FIG. 391 (SEQ ID NO: 391), FIG. 393 (SEQ ID NO: 393), FIG. 395 (SEQ ID NO: 395), FIG. 397 (SEQ ID NO: 397), FIG. 399 (SEQ ID NO: 399), FIG. 401 (SEQ ID NO: 401), FIG. 403 (SEQ ID NO: 403), FIG. 405 (SEQ ID NO: 405), FIG. 407 (SEQ ID NO: 407), FIG. 409 (SEQ ID NO: 409), FIG. 411 (SEQ ID NO: 411), FIG. 413 (SEQ ID NO: 413), FIG. 415 (SEQ ID NO: 415), FIG. 417 (SEQ ID NO: 417), FIG. 419 (SEQ ID NO: 419), FIG. 421 (SEQ ID NO: 421), FIG. 423 (SEQ ID NO: 423), FIG. 425 (SEQ ID NO: 425), FIG. 427 (SEQ ID NO: 427), FIG. 429 (SEQ ID NO: 429), FIG. 431 (SEQ ID NO: 431), FIG. 433 (SEQ ID NO: 433), FIG. 435 (SEQ ID NO: 435), FIG. 437 (SEQ ID NO: 437), FIG. 439 (SEQ ID NO: 439), FIG. 441 (SEQ ID NO: 441), FIG. 443 (SEQ ID NO: 443), FIG. 445 (SEQ ID NO: 445), FIG. 447 (SEQ ID NO: 447), FIG. 449 (SEQ ID NO: 449), FIG. 451 (SEQ ID NO: 451), FIG. 453 (SEQ ID NO: 453), FIG. 455 (SEQ ID NO: 455), FIG. 457 (SEQ ID NO: 457), FIG. 459 (SEQ ID NO: 459), FIG. 461 (SEQ ID NO: 461), FIG. 463 (SEQ ID NO: 463), FIG. 465 (SEQ ID NO: 465), FIG. 467 (SEQ ID NO: 467), FIG. 469 (SEQ ID NO: 469), FIG. 471 (SEQ ID NO: 471), FIG. 473 (SEQ ID NO: 473), FIG. 475 (SEQ ID NO: 475), FIG. 477 (SEQ ID NO: 477), FIG. 479 (SEQ ID NO: 479), FIG. 481 (SEQ ID NO: 481), FIG. 483 (SEQ ID NO: 483), FIG. 485 (SEQ ID NO: 485), FIG. 487 (SEQ ID NO: 487), FIG. 489 (SEQ ID NO: 489), FIG. 491 (SEQ ID NO: 491), FIG. 493 (SEQ ID NO: 493), FIG. 495 (SEQ ID NO: 495), FIG. 497 (SEQ ID NO: 497), FIG. 499 (SEQ ID NO: 499), FIG. 501 (SEQ ID NO: 501), FIG. 503 (SEQ ID NO: 503), FIG. 505 (SEQ ID NO: 505), FIG. 507 (SEQ ID NO: 507), FIG. 509 (SEQ ID NO: 509), FIG. 511 (SEQ ID NO: 511), FIG. 513 (SEQ ID NO: 513), FIG. 515 (SEQ ID NO: 515), FIG. 517 (SEQ ID NO: 517), FIG. 519 (SEQ ID NO: 519), FIG. 521 (SEQ ID NO: 521), FIG. 523 (SEQ ID NO: 523), FIGS. 525A-525B (SEQ ID NO: 525), FIG. 527 (SEQ ID NO: 527), FIG. 529 (SEQ ID NO: 529), FIG. 531 (SEQ ID NO: 531), FIG. 533 (SEQ ID NO: 533), FIG. 535 (SEQ ID NO: 535), FIG. 537 (SEQ ID NO: 537), FIG. 539 (SEQ ID NO: 539), FIG. 541 (SEQ ID NO: 541), FIG. 543 (SEQ ID NO: 543), FIG. 545 (SEQ ID NO: 545), FIG. 547 (SEQ ID NO: 547), FIG. 549 (SEQ ID NO: 549), FIG. 551 (SEQ ID NO: 551), FIG. 553 (SEQ ID NO: 553), FIG. 555 (SEQ ID NO: 555), FIG. 557 (SEQ ID NO: 557), FIG. 559 (SEQ ID NO: 559), FIG. 561 (SEQ ID NO: 561), FIG. 563 (SEQ ID NO: 563), FIG. 565 (SEQ ID NO: 565), FIG. 567 (SEQ ID NO: 567), FIG. 569 (SEQ ID NO: 569), FIG. 571 (SEQ ID NO: 571), FIG. 573 (SEQ ID NO: 573), FIG. 575 (SEQ ID NO: 575), FIG. 577 (SEQ ID NO: 577), FIG. 579 (SEQ ID NO: 579), FIG. 581 (SEQ ID NO: 581), FIG. 583 (SEQ ID NO: 583), FIG. 585 (SEQ ID NO: 585), FIG. 587 (SEQ ID NO: 587), FIG. 589 (SEQ ID NO: 589), FIG. 591 (SEQ ID NO: 591), FIG. 593 (SEQ ID NO: 593), FIG. 595 (SEQ ID NO: 595), FIG. 597 (SEQ ID NO: 597), FIG. 599 (SEQ ID NO: 599), FIG. 601 (SEQ ID NO: 601), FIG. 603 (SEQ ID NO: 603), FIG. 605 (SEQ ID NO: 605), FIG. 607 (SEQ ID NO: 607), and FIG. 609 (SEQ ID NO: 609).

4. Isolated nucleic acid having at least 80% nucleic acid sequence identity to the full-length coding sequence of the DNA deposited under any ATCC accession number shown in Table 7.

5. A vector comprising the nucleic acid of claim 1.

6. A host cell comprising the vector of claim 5.

7. The host cell of claim 6, wherein said cell is a CHO cell.

8. The host cell of claim 6, wherein said cell is an E. coli.

9. The host cell of claim 6, wherein said cell is a yeast cell.

10. A process for producing a PRO polypeptide comprising culturing the host cell of claim 6 under conditions suitable for expression of said PRO polypeptide and recovering said PRO polypeptide from the cell culture.

11. An isolated polypeptide having at least 80% amino acid sequence identity to an amino acid sequence selected from the group consisting of the amino acid sequence shown in FIG. 2 (SEQ ID NO: 2), FIG. 4 (SEQ ID NO: 4), FIG. 6 (SEQ ID NO: 6), FIG. 8 (SEQ ID NO: 8), FIG. 10 (SEQ ID NO: 10), FIG. 12 (SEQ ID NO: 12), FIG. 14 (SEQ ID NO: 14), FIG. 16 (SEQ ID NO: 16), FIG. 18 (SEQ ID NO: 18), FIG. 20 (SEQ ID NO: 20), FIG. 22 (SEQ ID NO: 22), FIG. 24 (SEQ ID NO: 24), FIG. 26 (SEQ ID NO: 26), FIG. 28 (SEQ ID NO: 28), FIG. 30 (SEQ ID NO: 30), FIG. 32 (SEQ ID NO: 32), FIG. 34 (SEQ ID NO: 34), FIG. 36 (SEQ ID NO: 36), FIG. 38 (SEQ ID NO: 38), FIG. 40 (SEQ ID NO: 40), FIG. 42 (SEQ ID NO: 42), FIG. 44 (SEQ ID NO: 44), FIG. 46 (SEQ ID NO: 46), FIG. 48 (SEQ ID NO: 48), FIG. 50 (SEQ ID NO: 50), FIG. 52 (SEQ ID NO: 52), FIG. 54 (SEQ ID NO: 54), FIG. 56 (SEQ ID NO: 56), FIG. 58 (SEQ ID NO: 58), FIG. 60 (SEQ ID NO: 60), FIG. 62 (SEQ ID NO: 62), FIG. 64 (SEQ ID NO: 64), FIG. 66 (SEQ ID NO: 66), FIG. 68 (SEQ ID NO: 68), FIG. 70 (SEQ ID NO: 70), FIG. 72 (SEQ ID NO: 72), FIG. 74 (SEQ ID NO: 74), FIG. 76 (SEQ ID NO: 76), FIG. 78 (SEQ ID NO: 78), FIG. 80 (SEQ ID NO: 80), FIG. 82 (SEQ ID NO: 82), FIG. 84 (SEQ ID NO: 84), FIG. 86 (SEQ ID NO: 86), FIG. 88 (SEQ ID NO: 88), FIG. 90 (SEQ ID NO: 90), FIG. 92 (SEQ ID NO: 92), FIG. 94 (SEQ ID NO: 94), FIG. 96 (SEQ ID NO: 96), FIG. 98 (SEQ ID NO: 98), FIG. 100 (SEQ ID NO: 100), FIG. 102 (SEQ ID NO: 102), FIG. 104 (SEQ ID NO: 104), FIG. 106 (SEQ ID NO: 106), FIG. 108 (SEQ ID NO: 108), FIG. 110 (SEQ ID NO: 110), FIG. 112 (SEQ ID NO: 112), FIG. 114 (SEQ ID NO: 114), FIG. 116 (SEQ ID NO: 116), FIG. 118 (SEQ ID NO: 118), FIG. 120 (SEQ ID NO: 120), FIG. 122 (SEQ ID NO: 122), FIG. 124 (SEQ ID NO: 124), FIG. 126 (SEQ ID NO: 126), FIG. 128 (SEQ ID NO: 128), FIG. 130 (SEQ ID NO: 130), FIG. 132 (SEQ ID NO: 132), FIG. 134 (SEQ ID NO: 134), FIG. 136 (SEQ ID NO: 136), FIG. 138 (SEQ ID NO: 138), FIG. 140 (SEQ ID NO: 140), FIG. 142 (SEQ ID NO: 142), FIG. 144 (SEQ ID NO: 144), FIG. 146 (SEQ ID NO: 146), FIG. 148 (SEQ ID NO: 148), FIG. 150 (SEQ ID NO: 150), FIG. 152 (SEQ ID NO: 152), FIG. 154 (SEQ ID NO: 154), FIG. 156 (SEQ ID NO: 156), FIG. 158 (SEQ ID NO: 158), FIG. 160 (SEQ ID NO: 160), FIG. 162 (SEQ ID NO: 162), FIG. 164 (SEQ ID NO: 164), FIG. 166 (SEQ ID NO: 166), FIG. 168 (SEQ ID NO: 168), FIG. 170 (SEQ ID NO: 170), FIG. 172 (SEQ ID NO: 172), FIG. 174 (SEQ ID NO: 174), FIG. 176 (SEQ ID NO: 176), FIG. 178 (SEQ ID NO: 178), FIG. 180 (SEQ ID NO: 180), FIG. 182 (SEQ ID NO: 182), FIG. 184 (SEQ ID NO: 184), FIG. 186 (SEQ ID NO: 186), FIG. 188 (SEQ ID NO: 188), FIG. 190 (SEQ ID NO: 190), FIG. 192 (SEQ ID NO: 192), FIG. 194 (SEQ ID NO: 194), FIG. 196 (SEQ ID NO: 196), FIG. 198 (SEQ ID NO: 198), FIG. 200 (SEQ ID NO: 200), FIG. 202 (SEQ ID NO: 202), FIG. 204 (SEQ ID NO: 204), FIG. 206 (SEQ ID NO: 206), FIG. 208 (SEQ ID NO: 208), FIG. 210 (SEQ ID NO: 210), FIG. 212 (SEQ ID NO: 212), FIG. 214 (SEQ ID NO: 214), FIG. 216 (SEQ ID NO: 216), FIG. 218 (SEQ ID NO: 218), FIG. 220 (SEQ ID NO: 220), FIG. 222 (SEQ ID NO: 222), FIG. 224 (SEQ ID NO: 224), FIG. 226 (SEQ ID NO: 226), FIG. 228 (SEQ ID NO: 228), FIG. 230 (SEQ ID NO: 230), FIG. 232 (SEQ ID NO: 232), FIG. 234 (SEQ ID NO: 234), FIG. 236 (SEQ ID NO: 236), FIG. 238 (SEQ ID NO: 238), FIG. 240 (SEQ ID NO: 240), FIG. 242 (SEQ ID NO: 242), FIG. 244 (SEQ ID NO: 244), FIG. 246 (SEQ ID NO: 246), FIG. 248 (SEQ ID NO: 248), FIG. 250 (SEQ ID NO: 250), FIG. 252 (SEQ ID NO: 252), FIG. 254 (SEQ ID NO: 254), FIG. 256 (SEQ ID NO: 256), FIG. 258 (SEQ ID NO: 258), FIG. 260 (SEQ ID NO: 260), FIG. 262 (SEQ ID NO: 262), FIG. 264 (SEQ ID NO: 264), FIG. 266 (SEQ ID NO: 266), FIG. 268 (SEQ ID NO: 268), FIG. 270 (SEQ ID NO: 270), FIG. 272 (SEQ ID NO: 272), FIG. 274 (SEQ ID NO: 274), FIG. 276 (SEQ ID NO: 276), FIG. 278 (SEQ ID NO: 278), FIG. 280 (SEQ ID NO: 280), FIG. 282 (SEQ ID NO: 282), FIG. 284 (SEQ ID NO: 284), FIG. 286 (SEQ ID NO: 286), FIG. 288 (SEQ ID NO: 288), FIG. 290 (SEQ ID NO: 290), FIG. 292 (SEQ ID NO: 292), FIG. 294 (SEQ ID NO: 294), FIG. 296 (SEQ ID NO: 296), FIG. 298 (SEQ ID NO: 298), FIG. 300 (SEQ ID NO: 300), FIG. 302 (SEQ ID NO: 302), FIG. 304 (SEQ ID NO: 304), FIG. 306 (SEQ ID NO: 306), FIG. 308 (SEQ ID NO: 308), FIG. 310 (SEQ ID NO: 310), FIG. 312 (SEQ ID NO: 312), FIG. 314 (SEQ ID NO: 314), FIG. 316 (SEQ ID NO: 316), FIG. 318 (SEQ ID NO: 318), FIG. 320 (SEQ ID NO: 320), FIG. 322 (SEQ ID NO: 322), FIG. 324 (SEQ ID NO: 324), FIG. 326 (SEQ ID NO: 326), FIG. 328 (SEQ ID NO: 328), FIG. 330 (SEQ ID NO: 330), FIG. 332 (SEQ ID NO: 332), FIG. 334 (SEQ ID NO: 334), FIG. 336 (SEQ ID NO: 336), FIG. 338 (SEQ ID NO: 338), FIG. 340 (SEQ ID NO: 340), FIG. 342 (SEQ ID NO: 342), FIG. 344 (SEQ ID NO: 344), FIG. 346 (SEQ ID NO: 346), FIG. 348 (SEQ ID NO: 348), FIG. 350 (SEQ ID NO: 350), FIG. 352 (SEQ ID NO: 352), FIG. 354 (SEQ ID NO: 354), FIG. 356 (SEQ ID NO: 356), FIG. 358 (SEQ ID NO: 358), FIG. 360 (SEQ ID NO: 360), FIG. 362 (SEQ ID NO: 362), FIG. 364 (SEQ ID NO: 364), FIG. 366 (SEQ ID NO: 366), FIG. 368 (SEQ ID NO: 368), FIG. 370 (SEQ ID NO: 370), FIG. 372 (SEQ ID NO: 372), FIG. 374 (SEQ ID NO: 374), FIG. 376 (SEQ ID NO: 376), FIG. 378 (SEQ ID NO: 378), FIG. 380 (SEQ ID NO: 380), FIG. 382 (SEQ ID NO: 382), FIG. 384 (SEQ ID NO: 384), FIG. 386 (SEQ ID NO: 386), FIG. 388 (SEQ ID NO: 388), FIG. 390 (SEQ ID NO: 390), FIG. 392 (SEQ ID NO: 392), FIG. 394 (SEQ ID NO: 394), FIG. 396 (SEQ ID NO: 396), FIG. 398 (SEQ ID NO: 398), FIG. 400 (SEQ ID NO: 400), FIG. 402 (SEQ ID NO: 402), FIG. 404 (SEQ ID NO: 404), FIG. 406 (SEQ ID NO: 406), FIG. 408 (SEQ ID NO: 408), FIG. 410 (SEQ ID NO: 410), FIG. 412 (SEQ ID NO: 412), FIG. 414 (SEQ ID NO: 414), FIG. 416 (SEQ ID NO: 416), FIG. 418 (SEQ ID NO: 418), FIG. 420 (SEQ ID NO: 420), FIG. 422 (SEQ ID NO: 422), FIG. 424 (SEQ ID NO: 424), FIG. 426 (SEQ ID NO: 426), FIG. 428 (SEQ ID NO: 428), FIG. 430 (SEQ ID NO: 430), FIG. 432 (SEQ ID NO: 432), FIG. 434 (SEQ ID NO: 434), FIG. 436 (SEQ ID NO: 436), FIG. 438 (SEQ ID NO: 438), FIG. 440 (SEQ ID NO: 440), FIG. 442 (SEQ ID NO: 442), FIG. 444 (SEQ ID NO: 444), FIG. 446 (SEQ ID NO: 446), FIG. 448 (SEQ ID NO: 448), FIG. 450 (SEQ ID NO: 450), FIG. 452 (SEQ ID NO: 452), FIG. 454 (SEQ ID NO: 454), FIG. 456 (SEQ ID NO: 456), FIG. 458 (SEQ ID NO: 458), FIG. 460 (SEQ ID NO: 460), FIG. 462 (SEQ ID NO: 462), FIG. 464 (SEQ ID NO: 464), FIG. 466 (SEQ ID NO: 466), FIG. 468 (SEQ ID NO: 468), FIG. 470 (SEQ ID NO: 470), FIG. 472 (SEQ ID NO: 472), FIG. 474 (SEQ ID NO: 474), FIG. 476 (SEQ ID NO: 476), FIG. 478 (SEQ ID NO: 478), FIG. 480 (SEQ ID NO: 480), FIG. 482 (SEQ ID NO: 482), FIG. 484 (SEQ ID NO: 484), FIG. 486 (SEQ ID NO: 486), FIG. 488 (SEQ ID NO: 488), FIG. 490 (SEQ ID NO: 490), FIG. 492 (SEQ ID NO: 492), FIG. 494 (SEQ ID NO: 494), FIG. 496 (SEQ ID NO: 496), FIG. 498 (SEQ ID NO: 498), FIG. 500 (SEQ ID NO: 500), FIG. 502 (SEQ ID NO: 502), FIG. 504 (SEQ ID NO: 504), FIG. 506 (SEQ ID NO: 506), FIG. 508 (SEQ ID NO: 508), FIG. 510 (SEQ ID NO: 510), FIG. 512 (SEQ ID NO: 512), FIG. 514 (SEQ ID NO: 514), FIG. 516 (SEQ ID NO: 516), FIG. 518 (SEQ ID NO: 518), FIG. 520 (SEQ ID NO: 520), FIG. 522 (SEQ ID NO: 522), FIG. 524 (SEQ ID NO: 524), FIG. 526 (SEQ ID NO: 526), FIG. 528 (SEQ ID NO: 528), FIG. 530 (SEQ ID NO: 530), FIG. 532 (SEQ ID NO: 532), FIG. 534 (SEQ ID NO: 534), FIG. 536 (SEQ ID NO: 536), FIG. 538 (SEQ ID NO: 538), FIG. 540 (SEQ ID NO: 540), FIG. 542 (SEQ ID NO: 542), FIG. 544 (SEQ ID NO: 544), FIG. 546 (SEQ ID NO: 546), FIG. 548 (SEQ ID NO: 548), FIG. 550 (SEQ ID NO: 550), FIG. 552 (SEQ ID NO: 552), FIG. 554 (SEQ ID NO: 554), FIG. 556 (SEQ ID NO: 556), FIG. 558 (SEQ ID NO: 558), FIG. 560 (SEQ ID NO: 560), FIG. 562 (SEQ ID NO: 562), FIG. 564 (SEQ ID NO: 564), FIG. 566 (SEQ ID NO: 566), FIG. 568 (SEQ ID NO: 568), FIG. 570 (SEQ ID NO: 570), FIG. 572 (SEQ ID NO: 572), FIG. 574 (SEQ ID NO: 574), FIG. 576 (SEQ ID NO: 576), FIG. 578 (SEQ ID NO: 578), FIG. 580 (SEQ ID NO: 580), FIG. 582 (SEQ ID NO: 582), FIG. 584 (SEQ ID NO: 584), FIG. 586 (SEQ ID NO: 586), FIG. 588 (SEQ ID NO: 588), FIG. 590 (SEQ ID NO: 590), FIG. 592 (SEQ ID NO: 592), FIG. 594 (SEQ ID NO: 594), FIG. 596 (SEQ ID NO: 596), FIG. 598 (SEQ ID NO: 598), FIG. 600 (SEQ ID NO: 600), FIG. 602 (SEQ ID NO: 602), FIG. 604 (SEQ ID NO: 604), FIG. 606 (SEQ ID NO: 606), FIG. 608 (SEQ ID NO: 608), and FIG. 610 (SEQ ID NO: 610).

12. An isolated polypeptide having at least 80% amino acid sequence identity to an amino acid sequence encoded by the full-length coding sequence of the DNA deposited under any ATCC accession number shown in Table 7.

13. A chimeric molecule comprising a polypeptide according to claim 11 fused to a heterologous amino acid sequence.

14. The chimeric molecule of claim 13, wherein said heterologous amino acid sequence is an epitope tag sequence.

15. The chimeric molecule of claim 13, wherein said heterologous amino acid sequence is a Fc region of an immunoglobulin.

16. An antibody which specifically binds to a polypeptide according to claim 11.

17. The antibody of claim 16, wherein said antibody is a monoclonal antibody, a humanized antibody or a single-chain antibody.

18. Isolated nucleic acid having at least 80% nucleic acid sequence identity to:
(a) a nucleotide sequence encoding the polypeptide shown in FIG. 2 (SEQ ID NO: 2), FIG. 4 (SEQ ID NO: 4), FIG. 6 (SEQ ID NO: 6), FIG. 8 (SEQ ID NO: 8), FIG. 10 (SEQ ID NO: 10), FIG. 12 (SEQ ID NO: 12), FIG. 14 (SEQ ID NO: 14), FIG. 16 (SEQ ID NO: 16), FIG. 18 (SEQ ID NO: 18), FIG. 20 (SEQ ID NO: 20), FIG. 22 (SEQ ID NO: 22), FIG. 24 (SEQ ID NO: 24), FIG. 26 (SEQ ID NO: 26), FIG. 28 (SEQ ID NO: 28), FIG. 30 (SEQ ID NO: 30), FIG. 32 (SEQ ID NO: 32), FIG. 34 (SEQ ID NO: 34), FIG. 36 (SEQ ID NO: 36), FIG. 38 (SEQ ID NO: 38), FIG. 40 (SEQ ID NO: 40), FIG. 42 (SEQ ID NO: 42), FIG. 44 (SEQ ID NO: 44), FIG. 46 (SEQ ID NO: 46), FIG. 48 (SEQ ID NO: 48), FIG. 50 (SEQ ID NO: 50), FIG. 52 (SEQ ID NO: 52), FIG. 54 (SEQ ID NO: 54), FIG. 56 (SEQ ID NO: 56), FIG. 58 (SEQ ID NO: 58), FIG. 60 (SEQ ID NO: 60), FIG. 62 (SEQ ID NO: 62), FIG. 64 (SEQ ID NO: 64), FIG. 66 (SEQ ID NO: 66), FIG. 68 (SEQ ID NO: 68), FIG. 70 (SEQ ID NO: 70), FIG. 72 (SEQ ID NO: 72), FIG. 74 (SEQ ID NO: 74), FIG. 76 (SEQ ID NO: 76), FIG. 78 (SEQ ID NO: 78), FIG. 80 (SEQ ID NO: 80), FIG. 82 (SEQ ID NO: 82), FIG. 84 (SEQ ID NO: 84), FIG. 86 (SEQ ID NO: 86), FIG. 88 (SEQ ID NO: 88), FIG. 90 (SEQ ID NO: 90), FIG. 92 (SEQ ID NO: 92), FIG. 94 (SEQ ID NO: 94), FIG. 96 (SEQ ID NO: 96), FIG. 98 (SEQ ID NO: 98), FIG. 100 (SEQ ID NO: 100), FIG. 102 (SEQ ID NO: 102), FIG. 104 (SEQ ID NO: 104), FIG. 106 (SEQ ID NO: 106), FIG. 108 (SEQ ID NO: 108), FIG. 110 (SEQ ID NO: 110), FIG. 112 (SEQ ID NO: 112), FIG. 114 (SEQ ID NO: 114), FIG. 116 (SEQ ID NO: 116), FIG. 118 (SEQ ID NO: 118), FIG. 120 (SEQ ID NO: 120), FIG. 122 (SEQ ID NO: 122), FIG. 124 (SEQ ID NO: 124), FIG. 126 (SEQ ID NO: 126), FIG. 128 (SEQ ID NO: 128), FIG. 130 (SEQ ID NO: 130), FIG. 132 (SEQ ID NO: 132), FIG. 134 (SEQ ID NO: 134), FIG. 136 (SEQ ID NO: 136), FIG. 138 (SEQ ID NO: 138), FIG. 140 (SEQ ID NO: 140), FIG. 142 (SEQ ID NO: 142), FIG. 144 (SEQ ID NO: 144), FIG. 146 (SEQ ID NO: 146), FIG. 148 (SEQ ID NO: 148), FIG. 150 (SEQ ID NO: 150), FIG. 152 (SEQ ID NO: 152), FIG. 154 (SEQ ID NO: 154), FIG. 156 (SEQ ID NO: 156), FIG. 158 (SEQ ID NO: 158), FIG. 160 (SEQ ID NO: 160), FIG. 162 (SEQ ID NO: 162), FIG. 164 (SEQ ID NO: 164), FIG. 166 (SEQ ID NO: 166), FIG. 168 (SEQ ID NO: 168), FIG. 170 (SEQ ID NO: 170), FIG. 172 (SEQ ID NO: 172), FIG. 174 (SEQ ID NO: 174), FIG. 176 (SEQ ID NO: 176), FIG. 178 (SEQ ID NO: 178), FIG. 180 (SEQ ID NO: 180), FIG. 182 (SEQ ID NO: 182), FIG. 184 (SEQ ID NO: 184), FIG. 186 (SEQ ID NO: 186), FIG. 188 (SEQ ID NO: 188), FIG. 190 (SEQ ID NO: 190), FIG. 192 (SEQ ID NO: 192), FIG. 194 (SEQ ID NO: 194), FIG. 196 (SEQ ID NO: 196), FIG. 198 (SEQ ID NO: 198), FIG. 200 (SEQ ID NO: 200), FIG. 202 (SEQ ID NO: 202), FIG. 204 (SEQ ID NO: 204), FIG. 206 (SEQ ID NO: 206), FIG. 208 (SEQ ID NO: 208), FIG. 210 (SEQ ID NO: 210), FIG. 212 (SEQ ID NO: 212), FIG. 214 (SEQ ID NO: 214), FIG. 216 (SEQ ID NO: 216), FIG. 218 (SEQ ID NO: 218), FIG. 220 (SEQ ID NO: 220), FIG. 222 (SEQ ID NO: 222), FIG. 224 (SEQ ID NO: 224), FIG. 226 (SEQ ID NO: 226), FIG. 228 (SEQ ID NO: 228), FIG. 230 (SEQ ID NO: 230), FIG. 232 (SEQ ID NO: 232), FIG. 234 (SEQ ID NO: 234), FIG. 236 (SEQ ID NO: 236), FIG. 238 (SEQ ID NO: 238), FIG. 240 (SEQ ID NO: 240), FIG. 242 (SEQ ID NO: 242), FIG. 244 (SEQ ID NO: 244), FIG. 246 (SEQ ID NO: 246), FIG. 248 (SEQ ID NO: 248), FIG. 250 (SEQ ID NO: 250), FIG. 252 (SEQ ID NO: 252), FIG. 254 (SEQ ID NO: 254), FIG. 256 (SEQ ID NO: 256), FIG. 258 (SEQ ID NO: 258), FIG. 260 (SEQ ID NO: 260), FIG. 262 (SEQ ID NO: 262), FIG. 264 (SEQ ID NO: 264), FIG. 266 (SEQ ID NO: 266), FIG. 268 (SEQ ID NO: 268), FIG. 270 (SEQ ID NO: 270), FIG. 272 (SEQ ID NO: 272), FIG. 274 (SEQ ID NO: 274), FIG. 276 (SEQ ID NO: 276), FIG. 278 (SEQ ID NO: 278), FIG. 280 (SEQ ID NO: 280), FIG. 282 (SEQ ID NO: 282), FIG. 284 (SEQ ID NO: 284), FIG. 286 (SEQ ID NO: 286), FIG. 288 (SEQ ID NO: 288), FIG. 290 (SEQ ID NO: 290), FIG. 292 (SEQ ID NO: 292), FIG. 294 (SEQ ID NO: 294), FIG. 296 (SEQ ID NO: 296), FIG. 298 (SEQ ID NO: 298), FIG. 300 (SEQ ID NO: 300), FIG. 302 (SEQ ID NO: 302), FIG. 304 (SEQ ID NO: 304), FIG. 306 (SEQ ID NO: 306), FIG. 308 (SEQ ID NO: 308), FIG. 310 (SEQ ID NO: 310), FIG. 312 (SEQ ID NO: 312), FIG. 314 (SEQ ID NO: 314), FIG. 316 (SEQ ID NO: 316), FIG. 318 (SEQ ID NO: 318), FIG. 320 (SEQ ID NO: 320), FIG. 322 (SEQ ID NO: 322), FIG. 324 (SEQ ID NO: 324), FIG. 326 (SEQ ID NO: 326), FIG. 328 (SEQ ID NO: 328), FIG. 330 (SEQ ID NO: 330), FIG. 332 (SEQ ID NO: 332), FIG. 334 (SEQ ID NO: 334), FIG. 336 (SEQ ID NO: 336), FIG. 338 (SEQ ID NO: 338), FIG. 340 (SEQ ID NO: 340), FIG. 342 (SEQ ID NO: 342), FIG. 344 (SEQ ID NO: 344), FIG. 346 (SEQ ID NO: 346), FIG. 348 (SEQ ID NO: 348), FIG. 350 (SEQ ID NO: 350), FIG. 352 (SEQ ID NO: 352), FIG. 354 (SEQ ID NO: 354), FIG. 356 (SEQ ID NO: 356), FIG. 358 (SEQ ID NO: 358), FIG. 360 (SEQ ID NO: 360), FIG. 362 (SEQ ID NO: 362), FIG. 364 (SEQ ID NO: 364), FIG. 366 (SEQ ID NO: 366), FIG. 368 (SEQ ID NO: 368), FIG. 370 (SEQ ID NO: 370), FIG. 372 (SEQ ID NO: 372), FIG. 374 (SEQ ID NO: 374), FIG. 376 (SEQ ID NO: 376), FIG. 378 (SEQ ID NO: 378), FIG. 380 (SEQ ID NO: 380), FIG. 382 (SEQ ID NO: 382), FIG. 384 (SEQ ID NO: 384), FIG. 386 (SEQ ID NO: 386), FIG. 388 (SEQ ID NO: 388), FIG. 390 (SEQ ID NO: 390), FIG. 392 (SEQ ID NO: 392), FIG. 394 (SEQ ID NO: 394), FIG. 396 (SEQ ID NO: 396), FIG. 398 (SEQ ID NO: 398), FIG. 400 (SEQ ID NO: 400), FIG. 402 (SEQ ID NO: 402), FIG. 404 (SEQ ID NO: 404), FIG. 406 (SEQ ID NO: 406), FIG. 408 (SEQ ID NO: 408), FIG. 410 (SEQ ID NO: 410), FIG. 412 (SEQ ID NO: 412), FIG. 414 (SEQ ID NO: 414), FIG. 416 (SEQ ID NO: 416), FIG. 418 (SEQ ID NO: 418), FIG. 420 (SEQ ID NO: 420), FIG. 422 (SEQ ID NO: 422), FIG. 424 (SEQ ID NO: 424), FIG. 426 (SEQ ID NO: 426), FIG. 428 (SEQ ID NO: 428), FIG. 430 (SEQ ID NO: 430), FIG. 432 (SEQ ID NO: 432), FIG. 434 (SEQ ID NO: 434), FIG. 436 (SEQ ID NO: 436), FIG. 438 (SEQ ID NO: 438), FIG. 440 (SEQ ID NO: 440), FIG. 442 (SEQ ID NO: 442), FIG. 444 (SEQ ID NO: 444), FIG. 446 (SEQ ID NO: 446), FIG. 448 (SEQ ID NO: 448), FIG. 450 (SEQ ID NO: 450), FIG. 452 (SEQ ID NO: 452), FIG. 454 (SEQ ID NO: 454), FIG. 456 (SEQ ID NO: 456), FIG. 458 (SEQ ID NO: 458), FIG. 460 (SEQ ID NO: 460), FIG. 462 (SEQ ID NO: 462), FIG. 464 (SEQ ID NO: 464), FIG. 466 (SEQ ID NO: 466), FIG. 468 (SEQ ID NO: 468), FIG. 470 (SEQ ID NO: 470), FIG. 472 (SEQ ID NO: 472), FIG. 474 (SEQ ID NO: 474), FIG. 476 (SEQ ID NO: 476), FIG. 478 (SEQ ID NO: 478), FIG. 480 (SEQ ID NO: 480), FIG. 482 (SEQ ID NO: 482), FIG. 484 (SEQ ID NO: 484), FIG. 486 (SEQ ID NO: 486), FIG. 488 (SEQ ID NO: 488), FIG. 490 (SEQ ID NO: 490), FIG. 492 (SEQ ID NO: 492), FIG. 494 (SEQ ID NO: 494), FIG. 496 (SEQ ID NO: 496), FIG. 498 (SEQ ID NO: 498), FIG. 500 (SEQ ID NO: 500), FIG. 502 (SEQ ID NO: 502), FIG. 504 (SEQ ID NO: 504), FIG. 506 (SEQ ID NO: 506), FIG. 508 (SEQ ID NO: 508), FIG. 510 (SEQ ID NO: 510), FIG. 512 (SEQ ID NO: 512), FIG. 514 (SEQ ID NO: 514), FIG. 516 (SEQ ID NO: 516), FIG. 518 (SEQ ID NO: 518), FIG. 520 (SEQ ID NO: 520), FIG. 522 (SEQ ID NO: 522), FIG. 524 (SEQ ID NO: 524), FIG. 526 (SEQ ID NO: 526), FIG. 528 (SEQ ID NO: 528), FIG. 530 (SEQ ID NO: 530), FIG. 532 (SEQ ID NO: 532), FIG. 534 (SEQ ID NO: 534), FIG. 536 (SEQ ID NO: 536), FIG. 538 (SEQ ID NO: 538), FIG. 540 (SEQ ID NO: 540), FIG. 542 (SEQ ID NO: 542), FIG. 544 (SEQ ID NO: 544), FIG. 546 (SEQ ID NO: 546), FIG. 548 (SEQ ID NO: 548), FIG. 550 (SEQ ID NO: 550), FIG. 552 (SEQ ID NO: 552), FIG. 554 (SEQ ID NO: 554), FIG. 556 (SEQ ID NO: 556), FIG. 558 (SEQ ID NO: 558), FIG. 560 (SEQ ID NO: 560), FIG. 562 (SEQ ID NO: 562), FIG. 564 (SEQ ID NO: 564), FIG. 566 (SEQ ID NO: 566), FIG. 568 (SEQ ID NO: 568), FIG. 570 (SEQ ID NO: 570), FIG. 572 (SEQ ID NO: 572), FIG. 574 (SEQ ID NO: 574), FIG. 576 (SEQ ID NO: 576), FIG. 578 (SEQ ID NO: 578), FIG. 580 (SEQ ID NO: 580), FIG. 582 (SEQ ID NO: 582), FIG. 584 (SEQ ID NO: 584), FIG. 586 (SEQ ID NO: 586), FIG. 588 (SEQ ID NO: 588), FIG. 590 (SEQ ID NO: 590), FIG. 592 (SEQ ID NO: 592), FIG. 594 (SEQ ID NO: 594), FIG. 596 (SEQ ID NO: 596), FIG. 598 (SEQ ID NO: 598), FIG. 600 (SEQ ID NO: 600), FIG. 602 (SEQ ID NO: 602), FIG. 604 (SEQ ID NO: 604), FIG. 606 (SEQ ID NO: 606), FIG. 608 (SEQ ID NO: 608), or FIG. 610 (SEQ ID NO: 610), lacking its associated signal peptide;
(b) a nucleotide sequence encoding an extracellular domain of the polypeptide shown in FIG. 2 (SEQ ID NO: 2), FIG. 4 (SEQ ID NO: 4), FIG. 6 (SEQ ID NO: 6), FIG. 8 (SEQ ID NO: 8), FIG. 10 (SEQ ID NO: 10), FIG. 12 (SEQ ID NO: 12), FIG. 14 (SEQ ID NO: 14), FIG. 16 (SEQ ID NO: 16), FIG. 18 (SEQ ID NO: 18), FIG. 20 (SEQ ID NO: 20), FIG. 22 (SEQ ID NO: 22), FIG. 24 (SEQ ID NO: 24), FIG. 26 (SEQ ID NO: 26), FIG. 28 (SEQ ID NO: 28), FIG. 30 (SEQ ID NO: 30), FIG. 32 (SEQ ID NO: 32), FIG. 34 (SEQ ID NO: 34), FIG. 36 (SEQ ID NO: 36), FIG. 38 (SEQ ID NO: 38), FIG. 40 (SEQ ID NO: 40), FIG. 42 (SEQ ID NO: 42), FIG. 44 (SEQ ID NO: 44), FIG. 46 (SEQ ID NO: 46), FIG. 48 (SEQ ID NO: 48), FIG. 50 (SEQ ID NO: 50), FIG. 52 (SEQ ID NO: 52), FIG. 54 (SEQ ID NO: 54), FIG. 56 (SEQ ID NO: 56), FIG. 58 (SEQ ID NO: 58), FIG. 60 (SEQ ID NO: 60), FIG. 62 (SEQ ID NO: 62), FIG. 64 (SEQ ID NO: 64), FIG. 66 (SEQ ID NO: 66), FIG. 68 (SEQ ID NO: 68), FIG. 70 (SEQ ID NO: 70), FIG. 72 (SEQ ID NO: 72), FIG. 74 (SEQ ID NO: 74), FIG. 76 (SEQ ID NO: 76), FIG. 78 (SEQ ID NO: 78), FIG. 80 (SEQ ID NO: 80), FIG. 82 (SEQ ID NO: 82), FIG. 84 (SEQ ID NO: 84), FIG. 86 (SEQ ID NO: 86), FIG. 88 (SEQ ID NO: 88), FIG. 90 (SEQ ID NO: 90), FIG. 92 (SEQ ID NO: 92), FIG. 94 (SEQ ID NO: 94), FIG. 96 (SEQ ID NO: 96), FIG. 98 (SEQ ID NO: 98), FIG. 100 (SEQ ID NO: 100), FIG. 102 (SEQ ID NO: 102), FIG. 104 (SEQ ID NO: 104), FIG. 106 (SEQ ID NO: 106), FIG. 108 (SEQ ID NO: 108), FIG. 110 (SEQ ID NO: 110), FIG. 112 (SEQ ID NO: 112), FIG. 114 (SEQ ID NO: 114), FIG. 116 (SEQ ID NO: 116), FIG. 118 (SEQ ID NO: 118), FIG. 120 (SEQ ID NO: 120), FIG. 122 (SEQ ID NO: 122), FIG. 124 (SEQ ID NO: 124), FIG. 126 (SEQ ID NO: 126), FIG. 128 (SEQ ID NO: 128), FIG. 130 (SEQ ID NO: 130), FIG. 132 (SEQ ID NO: 132), FIG. 134 (SEQ ID NO: 134), FIG. 136 (SEQ ID NO: 136), FIG. 138 (SEQ ID NO: 138), FIG. 140 (SEQ ID NO: 140), FIG. 142 (SEQ ID NO: 142), FIG. 144 (SEQ ID NO: 144), FIG. 146 (SEQ ID NO: 146), FIG. 148 (SEQ ID NO: 148), FIG. 150 (SEQ ID NO: 150), FIG. 152 (SEQ ID NO: 152), FIG. 154 (SEQ ID NO: 154), FIG. 156 (SEQ ID NO: 156), FIG. 158 (SEQ ID NO: 158), FIG. 160 (SEQ ID NO: 160), FIG. 162 (SEQ ID NO: 162), FIG. 164 (SEQ ID NO: 164), FIG. 166 (SEQ ID NO: 166), FIG. 168 (SEQ ID NO: 168), FIG. 170 (SEQ ID NO: 170), FIG. 172 (SEQ ID NO: 172), FIG. 174 (SEQ ID NO: 174), FIG. 176 (SEQ ID NO: 176), FIG. 178 (SEQ ID NO: 178), FIG. 180 (SEQ ID NO: 180), FIG. 182 (SEQ ID NO: 182), FIG. 184 (SEQ ID NO: 184), FIG. 186 (SEQ ID NO: 186), FIG. 188 (SEQ ID NO: 188), FIG. 190 (SEQ ID NO: 190), FIG. 192 (SEQ ID NO: 192), FIG. 194 (SEQ ID NO: 194), FIG. 196 (SEQ ID NO: 196), FIG. 198 (SEQ ID NO: 198), FIG. 200 (SEQ ID NO: 200), FIG. 202 (SEQ ID NO: 202), FIG. 204 (SEQ ID NO: 204), FIG. 206 (SEQ ID NO: 206), FIG. 208 (SEQ ID NO: 208), FIG. 210 (SEQ ID NO: 210), FIG. 212 (SEQ ID NO: 212), FIG. 214 (SEQ ID NO: 214), FIG. 216 (SEQ ID NO: 216), FIG. 218 (SEQ ID NO: 218), FIG. 220 (SEQ ID NO: 220), FIG. 222 (SEQ ID NO: 222), FIG. 224 (SEQ ID NO: 224), FIG. 226 (SEQ ID NO: 226), FIG. 228 (SEQ ID NO: 228), FIG. 230 (SEQ ID NO: 230), FIG. 232 (SEQ ID NO: 232), FIG. 234 (SEQ ID NO: 234), FIG. 236 (SEQ ID NO: 236), FIG. 238 (SEQ ID NO: 238), FIG. 240 (SEQ ID NO: 240), FIG. 242 (SEQ ID NO: 242), FIG. 244 (SEQ ID NO: 244), FIG. 246 (SEQ ID NO: 246), FIG. 248 (SEQ ID NO: 248), FIG. 250 (SEQ ID NO: 250), FIG. 252 (SEQ ID NO: 252), FIG. 254 (SEQ ID NO: 254), FIG. 256 (SEQ ID NO: 256), FIG. 258 (SEQ ID NO: 258), FIG. 260 (SEQ ID NO: 260), FIG. 262 (SEQ ID NO: 262), FIG. 264 (SEQ ID NO: 264), FIG. 266 (SEQ ID NO: 266), FIG. 268 (SEQ ID NO: 268), FIG. 270 (SEQ ID NO: 270), FIG. 272 (SEQ ID NO: 272), FIG. 274 (SEQ ID NO: 274), FIG. 276 (SEQ ID NO: 276), FIG. 278 (SEQ ID NO: 278), FIG. 280 (SEQ ID NO: 280), FIG. 282 (SEQ ID NO: 282), FIG. 284 (SEQ ID NO: 284), FIG. 286 (SEQ ID NO: 286), FIG. 288 (SEQ ID NO: 288), FIG. 290 (SEQ ID NO: 290), FIG. 292 (SEQ ID NO: 292), FIG. 294 (SEQ ID NO: 294), FIG. 296 (SEQ ID NO: 296), FIG. 298 (SEQ ID NO: 298), FIG. 300 (SEQ ID NO: 300), FIG. 302 (SEQ ID NO: 302), FIG. 304 (SEQ ID NO: 304), FIG. 306 (SEQ ID NO: 306), FIG. 308 (SEQ ID NO: 308), FIG. 310 (SEQ ID NO: 310), FIG. 312 (SEQ ID NO: 312), FIG. 314 (SEQ ID NO: 314), FIG. 316 (SEQ ID NO: 316), FIG. 318 (SEQ ID NO: 318), FIG. 320 (SEQ ID NO: 320), FIG. 322 (SEQ ID NO: 322), FIG. 324 (SEQ ID NO: 324), FIG. 326 (SEQ ID NO: 326), FIG. 328 (SEQ ID NO: 328), FIG. 330 (SEQ ID NO: 330), FIG. 332 (SEQ ID NO: 332), FIG. 334 (SEQ ID NO: 334), FIG. 336 (SEQ ID NO: 336), FIG. 338 (SEQ ID NO: 338), FIG. 340 (SEQ ID NO: 340), FIG. 342 (SEQ ID NO: 342), FIG. 344 (SEQ ID NO: 344), FIG. 346 (SEQ ID NO: 346), FIG. 348 (SEQ ID NO: 348), FIG. 350 (SEQ ID NO: 350), FIG. 352 (SEQ ID NO: 352), FIG. 354 (SEQ ID NO: 354), FIG. 356 (SEQ ID NO: 356), FIG. 358 (SEQ ID NO: 358), FIG. 360 (SEQ ID NO: 360), FIG. 362 (SEQ ID NO: 362), FIG. 364 (SEQ ID NO: 364), FIG. 366 (SEQ ID NO: 366), FIG. 368 (SEQ ID NO: 368), FIG. 370 (SEQ ID NO: 370), FIG. 372 (SEQ ID NO: 372), FIG. 374 (SEQ ID NO: 374), FIG. 376 (SEQ ID NO: 376), FIG. 378 (SEQ ID NO: 378), FIG. 380 (SEQ ID NO: 380), FIG. 382 (SEQ ID NO: 382), FIG. 384 (SEQ ID NO: 384), FIG. 386 (SEQ ID NO: 386), FIG. 388 (SEQ ID NO: 388), FIG. 390 (SEQ ID NO: 390), FIG. 392 (SEQ ID NO: 392), FIG. 394 (SEQ ID NO: 394), FIG. 396 (SEQ ID NO: 396), FIG. 398 (SEQ ID NO: 398), FIG. 400 (SEQ ID NO: 400), FIG. 402 (SEQ ID NO: 402), FIG. 404 (SEQ ID NO: 404), FIG. 406 (SEQ ID NO: 406), FIG. 408 (SEQ ID NO: 408), FIG. 410 (SEQ ID NO: 410), FIG. 412 (SEQ ID NO: 412), FIG. 414 (SEQ ID NO: 414), FIG. 416 (SEQ ID NO: 416), FIG. 418 (SEQ ID NO: 418), FIG. 420 (SEQ ID NO: 420), FIG. 422 (SEQ ID NO: 422), FIG. 424 (SEQ ID NO: 424), FIG. 426 (SEQ ID NO: 426), FIG. 428 (SEQ ID NO: 428), FIG. 430 (SEQ ID NO: 430), FIG. 432 (SEQ ID NO: 432), FIG. 434 (SEQ ID NO: 434), FIG. 436 (SEQ ID NO: 436), FIG. 438 (SEQ ID NO: 438), FIG. 440 (SEQ ID NO: 440), FIG. 442 (SEQ ID NO: 442), FIG. 444 (SEQ ID NO: 444), FIG. 446 (SEQ ID NO: 446), FIG. 448 (SEQ ID NO: 448), FIG. 450 (SEQ ID NO: 450), FIG. 452 (SEQ ID NO: 452), FIG. 454 (SEQ ID NO: 454), FIG. 456 (SEQ ID NO: 456), FIG. 458 (SEQ ID NO: 458), FIG. 460 (SEQ ID NO: 460), FIG. 462 (SEQ ID NO: 462), FIG. 464 (SEQ ID NO: 464), FIG. 466 (SEQ ID NO: 466), FIG. 468 (SEQ ID NO: 468), FIG. 470 (SEQ ID NO: 470), FIG. 472 (SEQ ID NO: 472), FIG. 474 (SEQ ID NO: 474), FIG. 476 (SEQ ID NO: 476), FIG. 478 (SEQ ID NO: 478), FIG. 480 (SEQ ID NO: 480), FIG. 482 (SEQ ID NO: 482), FIG. 484 (SEQ ID NO: 484), FIG. 486 (SEQ ID NO: 486), FIG. 488 (SEQ ID NO: 488), FIG. 490 (SEQ ID NO: 490), FIG. 492 (SEQ ID NO: 492), FIG. 494 (SEQ ID NO: 494), FIG. 496 (SEQ ID NO: 496), FIG. 498 (SEQ ID NO: 498), FIG. 500 (SEQ ID NO: 500), FIG. 502 (SEQ ID NO: 502), FIG. 504 (SEQ ID NO: 504), FIG. 506 (SEQ ID NO: 506), FIG. 508 (SEQ ID NO: 508), FIG. 510 (SEQ ID NO: 510), FIG. 512 (SEQ ID NO: 512), FIG. 514 (SEQ ID NO: 514), FIG. 516 (SEQ ID NO: 516), FIG. 518 (SEQ ID NO: 518), FIG. 520 (SEQ ID NO: 520), FIG. 522 (SEQ ID NO: 522), FIG. 524 (SEQ ID NO: 524), FIG. 526 (SEQ ID NO: 526), FIG. 528 (SEQ ID NO: 528), FIG. 530 (SEQ ID NO: 530), FIG. 532 (SEQ ID NO: 532), FIG. 534 (SEQ ID NO: 534), FIG. 536 (SEQ ID NO: 536), FIG. 538 (SEQ ID NO: 538), FIG. 540 (SEQ ID NO: 540), FIG. 542 (SEQ ID NO: 542), FIG. 544 (SEQ ID NO: 544), FIG. 546 (SEQ ID NO: 546), FIG. 548 (SEQ ID NO: 548), FIG. 550 (SEQ ID NO: 550), FIG. 552 (SEQ ID NO: 552), FIG. 554 (SEQ ID NO: 554), FIG. 556 (SEQ ID NO: 556), FIG. 558 (SEQ ID NO: 558), FIG. 560 (SEQ ID NO: 560), FIG. 562 (SEQ ID NO: 562), FIG. 564 (SEQ ID NO: 564), FIG. 566 (SEQ ID NO: 566), FIG. 568 (SEQ ID NO: 568), FIG. 570 (SEQ ID NO: 570), FIG. 572 (SEQ ID NO: 572), FIG. 574 (SEQ ID NO: 574), FIG. 576 (SEQ ID NO: 576), FIG. 578 (SEQ ID NO: 578), FIG. 580 (SEQ ID NO: 580), FIG. 582 (SEQ ID NO: 582), FIG. 584 (SEQ ID NO: 584), FIG. 586 (SEQ ID NO: 586), FIG. 588 (SEQ ID NO: 588), FIG. 590 (SEQ ID NO: 590), FIG. 592 (SEQ ID NO: 592), FIG. 594 (SEQ ID NO: 594), FIG. 596 (SEQ ID NO: 596), FIG. 598 (SEQ ID NO: 598), FIG. 600 (SEQ ID NO: 600), FIG. 602 (SEQ ID NO: 602), FIG. 604 (SEQ ID NO: 604), FIG. 606 (SEQ ID NO: 606), FIG. 608 (SEQ ID NO: 608), or FIG. 610 (SEQ ID NO: 610), with its associated signal peptide; or
(c) a nucleotide sequence encoding an extracellular domain of the polypeptide shown in FIG. 2 (SEQ ID NO: 2), FIG. 4 (SEQ ID NO: 4), FIG. 6 (SEQ ID NO: 6), FIG. 8 (SEQ ID NO: 8), FIG. 10 (SEQ ID NO: 10), FIG. 12 (SEQ ID NO: 12), FIG. 14 (SEQ ID NO: 14), FIG. 16 (SEQ ID NO: 16), FIG. 18 (SEQ ID NO: 18), FIG. 20 (SEQ ID NO: 20), FIG. 22 (SEQ ID NO: 22), FIG. 24 (SEQ ID NO: 24), FIG. 26 (SEQ ID NO: 26), FIG. 28 (SEQ ID NO: 28), FIG. 30 (SEQ ID NO: 30), FIG. 32 (SEQ ID NO: 32), FIG. 34 (SEQ ID NO: 34), FIG. 36 (SEQ ID NO: 36), FIG. 38 (SEQ ID NO: 38), FIG. 40 (SEQ ID NO: 40), FIG. 42 (SEQ ID NO: 42), FIG. 44 (SEQ ID NO: 44), FIG. 46 (SEQ ID NO: 46), FIG. 48 (SEQ ID NO: 48), FIG. 50 (SEQ ID NO: 50), FIG. 52 (SEQ ID NO: 52), FIG. 54 (SEQ ID NO: 54), FIG. 56 (SEQ ID NO: 56), FIG. 58 (SEQ ID NO: 58), FIG. 60 (SEQ ID NO: 60), FIG. 62 (SEQ ID NO: 62), FIG. 64 (SEQ ID NO: 64), FIG. 66 (SEQ ID NO: 66), FIG. 68 (SEQ ID NO: 68), FIG. 70 (SEQ ID NO: 70), FIG. 72 (SEQ ID NO: 72), FIG. 74 (SEQ ID NO: 74), FIG. 76 (SEQ ID NO: 76), FIG. 78 (SEQ ID NO: 78), FIG. 80 (SEQ ID NO: 80), FIG. 82 (SEQ ID NO: 82), FIG. 84 (SEQ ID NO: 84), FIG. 86 (SEQ ID NO: 86), FIG. 88 (SEQ ID NO: 88), FIG. 90 (SEQ ID NO: 90), FIG. 92 (SEQ ID NO: 92), FIG. 94 (SEQ ID NO: 94), FIG. 96 (SEQ ID NO: 96), FIG. 98 (SEQ ID NO: 98), FIG. 100 (SEQ ID NO: 100), FIG. 102 (SEQ ID NO: 102), FIG. 104 (SEQ ID NO: 104), FIG. 106 (SEQ ID NO: 106), FIG. 108 (SEQ ID NO: 108), FIG. 110 (SEQ ID NO: 110), FIG. 112 (SEQ ID NO: 112), FIG. 114 (SEQ ID NO: 114), FIG. 116 (SEQ ID NO: 116), FIG. 118 (SEQ ID NO: 118), FIG. 120 (SEQ ID NO: 120), FIG. 122 (SEQ ID NO: 122), FIG. 124 (SEQ ID NO: 124), FIG. 126 (SEQ ID NO: 126), FIG. 128 (SEQ ID NO: 128), FIG. 130 (SEQ ID NO: 130), FIG. 132 (SEQ ID NO: 132), FIG. 134 (SEQ ID NO: 134), FIG. 136 (SEQ ID NO: 136), FIG. 138 (SEQ ID NO: 138), FIG. 140 (SEQ ID NO: 140), FIG. 142 (SEQ ID NO: 142), FIG. 144 (SEQ ID NO: 144), FIG. 146 (SEQ ID NO: 146), FIG. 148 (SEQ ID NO: 148), FIG. 150 (SEQ ID NO: 150), FIG. 152 (SEQ ID NO: 152), FIG. 154 (SEQ ID NO: 154), FIG. 156 (SEQ ID NO: 156), FIG. 158 (SEQ ID NO: 158), FIG. 160 (SEQ ID NO: 160), FIG. 162 (SEQ ID NO: 162), FIG. 164 (SEQ ID NO: 164), FIG. 166 (SEQ ID NO: 166), FIG. 168 (SEQ ID NO: 168), FIG. 170 (SEQ ID NO: 170), FIG. 172 (SEQ ID NO: 172), FIG. 174 (SEQ ID NO: 174), FIG. 176 (SEQ ID NO: 176), FIG. 178 (SEQ ID NO: 178), FIG. 180 (SEQ ID NO: 180), FIG. 182 (SEQ ID NO: 182), FIG. 184 (SEQ ID NO: 184), FIG. 186 (SEQ ID NO: 186), FIG. 188 (SEQ ID NO: 188), FIG. 190 (SEQ ID NO: 190), FIG. 192 (SEQ ID NO: 192), FIG. 194 (SEQ ID NO: 194), FIG. 196 (SEQ ID NO: 196), FIG. 198 (SEQ ID NO: 198), FIG. 200 (SEQ ID NO: 200), FIG. 202 (SEQ ID NO: 202), FIG. 204 (SEQ ID NO: 204), FIG. 206 (SEQ ID NO: 206), FIG. 208 (SEQ ID NO: 208), FIG. 210 (SEQ ID NO: 210), FIG. 212 (SEQ ID NO: 212), FIG. 214 (SEQ ID NO: 214), FIG. 216 (SEQ ID NO: 216), FIG. 218 (SEQ ID NO: 218), FIG. 220 (SEQ ID NO: 220), FIG. 222 (SEQ ID NO: 222), FIG. 224 (SEQ ID NO: 224), FIG. 226 (SEQ ID NO: 226), FIG. 228 (SEQ ID NO: 228), FIG. 230 (SEQ ID NO: 230), FIG. 232 (SEQ ID NO: 232), FIG. 234 (SEQ ID NO: 234), FIG. 236 (SEQ ID NO: 236), FIG. 238 (SEQ ID NO: 238), FIG. 240 (SEQ ID NO: 240), FIG. 242 (SEQ ID NO: 242), FIG. 244 (SEQ ID NO: 244), FIG. 246 (SEQ ID NO: 246), FIG. 248 (SEQ ID NO: 248), FIG. 250 (SEQ ID NO: 250), FIG. 252 (SEQ ID NO: 252), FIG. 254 (SEQ ID NO: 254), FIG. 256 (SEQ ID NO: 256), FIG. 258 (SEQ ID NO: 258), FIG. 260 (SEQ ID NO: 260), FIG. 262 (SEQ ID NO: 262), FIG. 264 (SEQ ID NO: 264), FIG. 266 (SEQ ID NO: 266), FIG. 268 (SEQ ID NO: 268), FIG. 270 (SEQ ID NO: 270), FIG. 272 (SEQ ID NO: 272), FIG. 274 (SEQ ID NO: 274), FIG. 276 (SEQ ID NO: 276), FIG. 278 (SEQ ID NO: 278), FIG. 280 (SEQ ID NO: 280), FIG. 282 (SEQ ID NO: 282), FIG. 284 (SEQ ID NO: 284), FIG. 286 (SEQ ID NO: 286), FIG. 288 (SEQ ID NO: 288), FIG. 290 (SEQ ID NO: 290), FIG. 292 (SEQ ID NO: 292), FIG. 294 (SEQ ID NO: 294), FIG. 296 (SEQ ID NO: 296), FIG. 298 (SEQ ID NO: 298), FIG. 300 (SEQ ID NO: 300), FIG. 302 (SEQ ID NO: 302), FIG. 304 (SEQ ID NO: 304), FIG. 306 (SEQ ID NO: 306), FIG. 308 (SEQ ID NO: 308), FIG. 310 (SEQ ID NO: 310), FIG. 312 (SEQ ID NO: 312), FIG. 314 (SEQ ID NO: 314), FIG. 316 (SEQ ID NO: 316), FIG. 318 (SEQ ID NO: 318), FIG. 320 (SEQ ID NO: 320), FIG. 322 (SEQ ID NO: 322), FIG. 324 (SEQ ID NO: 324), FIG. 326 (SEQ ID NO: 326), FIG. 328 (SEQ ID NO: 328), FIG. 330 (SEQ ID NO: 330), FIG. 332 (SEQ ID NO: 332), FIG. 334 (SEQ ID NO: 334), FIG. 336 (SEQ ID NO: 336), FIG. 338 (SEQ ID NO: 338), FIG. 340 (SEQ ID NO: 340), FIG. 342 (SEQ ID NO: 342), FIG. 344 (SEQ ID NO: 344), FIG. 346 (SEQ ID NO: 346), FIG. 348 (SEQ ID NO: 348), FIG. 350 (SEQ ID NO: 350), FIG. 352 (SEQ ID NO: 352), FIG. 354 (SEQ ID NO: 354), FIG. 356 (SEQ ID NO: 356), FIG. 358 (SEQ ID NO: 358), FIG. 360 (SEQ ID NO: 360), FIG. 362 (SEQ ID NO: 362), FIG. 364 (SEQ ID NO: 364), FIG. 366 (SEQ ID NO: 366), FIG. 368 (SEQ ID NO: 368), FIG. 370 (SEQ ID NO: 370), FIG. 372 (SEQ ID NO: 372), FIG. 374 (SEQ ID NO: 374), FIG. 376 (SEQ ID NO: 376), FIG. 378 (SEQ ID NO: 378), FIG. 380 (SEQ ID NO: 380), FIG. 382 (SEQ ID NO: 382), FIG. 384 (SEQ ID NO: 384), FIG. 386 (SEQ ID NO: 386), FIG. 388 (SEQ ID NO: 388), FIG. 390 (SEQ ID NO: 390), FIG. 392 (SEQ ID NO: 392), FIG. 394 (SEQ ID NO: 394), FIG. 396 (SEQ ID NO: 396), FIG. 398 (SEQ ID NO: 398), FIG. 400 (SEQ ID NO : 400), FIG. 402 (SEQ ID NO: 402), FIG. 404 (SEQ ID NO: 404), FIG. 406 (SEQ ID NO: 406), FIG. 408 (SEQ ID NO: 408), FIG. 410 (SEQ ID NO: 410), FIG. 412 (SEQ ID NO: 412), FIG. 414 (SEQ ID NO: 414), FIG. 416 (SEQ ID NO: 416), FIG. 418 (SEQ ID NO: 418), FIG. 420 (SEQ ID NO: 420), FIG. 422 (SEQ ID NO: 422), FIG. 424 (SEQ ID NO: 424), FIG. 426 (SEQ ID NO: 426), FIG. 428 (SEQ ID NO: 428), FIG. 430 (SEQ ID NO: 430), FIG. 432 (SEQ ID NO: 432), FIG. 434 (SEQ ID NO: 434), FIG. 436 (SEQ ID NO: 436), FIG. 438 (SEQ ID NO: 438), FIG. 440 (SEQ ID NO: 440), FIG. 442 (SEQ ID NO: 442), FIG. 444 (SEQ ID NO: 444), FIG. 446 (SEQ ID NO: 446), FIG. 448 (SEQ ID NO: 448), FIG. 450 (SEQ ID NO: 450), FIG. 452 (SEQ ID NO: 452), FIG. 454 (SEQ ID NO: 454), FIG. 456 (SEQ ID NO: 456), FIG. 458 (SEQ ID NO: 458), FIG. 460 (SEQ ID NO: 460), FIG. 462 (SEQ ID NO: 462), FIG. 464 (SEQ ID NO: 464), FIG. 466 (SEQ ID NO: 466), FIG. 468 (SEQ ID NO: 468), FIG. 470 (SEQ ID NO: 470), FIG. 472 (SEQ ID NO: 472), FIG. 474 (SEQ ID NO: 474), FIG. 476 (SEQ ID NO: 476), FIG. 478 (SEQ ID NO: 478), FIG. 480 (SEQ ID NO: 480), FIG. 482 (SEQ ID NO: 482), FIG. 484 (SEQ ID NO: 484), FIG. 486 (SEQ ID NO: 486), FIG. 488 (SEQ ID NO: 488), FIG. 490 (SEQ ID NO: 490), FIG. 492 (SEQ ID NO: 492), FIG. 494 (SEQ ID NO: 494), FIG. 496 (SEQ ID NO: 496), FIG. 498 (SEQ ID NO: 498), FIG. 500 (SEQ ID NO: 500), FIG. 502 (SEQ ID NO: 502), FIG. 504 (SEQ ID NO: 504), FIG. 506 (SEQ ID NO: 506), FIG. 508 (SEQ ID NO: 508), FIG. 510 (SEQ ID NO: 5 10), FIG. 512 (SEQ ID NO: 512), FIG. 514 (SEQ ID NO: 514), FIG. 516 (SEQ ID NO: 516), FIG. 518 (SEQ ID NO: 518), FIG. 520 (SEQ ID NO: 520), FIG. 522 (SEQ ID NO: 522), FIG. 524 (SEQ ID NO: 524), FIG. 526 (SEQ ID NO: 526), FIG. 528 (SEQ ID NO: 528), FIG. 530 (SEQ ID NO: 530), FIG. 532 (SEQ ID NO: 532), FIG. 534 (SEQ ID NO: 534), FIG. 536 (SEQ ID NO: 536), FIG. 538 (SEQ ID NO: 538), FIG. 540 (SEQ ID NO: 540), FIG. 542 (SEQ ID NO: 542), FIG. 544 (SEQ ID NO: 544), FIG. 546 (SEQ ID NO: 546), FIG. 548 (SEQ ID NO: 548), FIG. 550 (SEQ ID NO: 550), FIG. 552 (SEQ ID NO: 552), FIG. 554 (SEQ ID NO: 554), FIG. 556 (SEQ ID NO: 556), FIG. 558 (SEQ ID NO: 558), FIG. 560 (SEQ ID NO: 560), FIG. 562 (SEQ ID NO: 562), FIG. 564 (SEQ ID NO: 564), FIG. 566 (SEQ ID NO: 566), FIG. 568 (SEQ ID NO: 568), FIG. 570 (SEQ ID NO: 570), FIG. 572 (SEQ ID NO: 572), FIG. 574 (SEQ ID NO: 574), FIG. 576 (SEQ ID NO: 576), FIG. 578 (SEQ ID NO: 578), FIG. 580 (SEQ ID NO: 580), FIG. 582 (SEQ ID NO: 582), FIG. 584 (SEQ ID NO: 584), FIG. 586 (SEQ ID NO: 586), FIG. 588 (SEQ ID NO: 588), FIG. 590 (SEQ ID NO: 590), FIG. 592 (SEQ ID NO: 592), FIG. 594 (SEQ ID NO: 594), FIG. 596 (SEQ ID NO: 596), FIG. 598 (SEQ ID NO: 598), FIG. 600 (SEQ ID NO: 600), FIG. 602 (SEQ ID NO: 602), FIG. 604 (SEQ ID NO: 604), FIG. 606 (SEQ ID NO: 606), FIG. 608 (SEQ ID NO: 608), or FIG. 610 (SEQ ID NO: 610), lacking its associated signal peptide.

19. An isolated polypeptide having at least 80% amino acid sequence identity to:
(a) an amino acid sequence of the polypeptide shown in FIG. 2 (SEQ ID NO: 2), FIG. 4 (SEQ ID NO: 4), FIG. 6 (SEQ ID NO: 6), FIG. 8 (SEQ ID NO: 8), FIG. 10 (SEQ ID NO: 10), FIG. 12 (SEQ ID NO: 12), FIG. 14 (SEQ ID NO: 14), FIG. 16 (SEQ ID NO: 16), FIG. 18 (SEQ ID NO: 18), FIG. 20 (SEQ ID NO: 20), FIG. 22 (SEQ ID NO: 22), FIG. 24 (SEQ ID NO: 24), FIG. 26 (SEQ ID NO: 26), FIG. 28 (SEQ ID NO: 28), FIG. 30 (SEQ ID NO: 30), FIG. 32 (SEQ ID NO: 32), FIG. 34 (SEQ ID NO: 34), FIG. 36 (SEQ ID NO: 36), FIG. 38 (SEQ ID NO: 38), FIG. 40 (SEQ ID NO: 40), FIG. 42 (SEQ ID NO: 42), FIG. 44 (SEQ ID NO: 44), FIG. 46 (SEQ ID NO: 46), FIG. 48 (SEQ ID NO: 48), FIG. 50 (SEQ ID NO: 50), FIG. 52 (SEQ ID NO: 52), FIG. 54 (SEQ ID NO: 54), FIG. 56 (SEQ ID NO: 56), FIG. 58 (SEQ ID NO: 58), FIG. 60 (SEQ ID NO: 60), FIG. 62 (SEQ ID NO: 62), FIG. 64 (SEQ ID NO: 64), FIG. 66 (SEQ ID NO: 66), FIG. 68 (SEQ ID NO: 68), FIG. 70 (SEQ ID NO: 70), FIG. 72 (SEQ ID NO: 72), FIG. 74 (SEQ ID NO: 74), FIG. 76 (SEQ ID NO: 76), FIG. 78 (SEQ ID NO: 78), FIG. 80 (SEQ ID NO: 80), FIG. 82 (SEQ ID NO: 82), FIG. 84 (SEQ ID NO: 84), FIG. 86 (SEQ ID NO: 86), FIG. 88 (SEQ ID NO: 88), FIG. 90 (SEQ ID NO: 90), FIG. 92 (SEQ ID NO: 92), FIG. 94 (SEQ ID NO: 94), FIG. 96 (SEQ ID NO: 96), FIG. 98 (SEQ ID NO: 98), FIG. 100 (SEQ ID NO: 100), FIG. 102 (SEQ ID NO: 102), FIG. 104 (SEQ ID NO: 104), FIG. 106 (SEQ ID NO: 106), FIG. 108 (SEQ ID NO: 108), FIG. 110 (SEQ ID NO: 110), FIG. 112 (SEQ ID NO: 112), FIG. 114 (SEQ ID NO: 114), FIG. 116 (SEQ ID NO: 116), FIG. 118 (SEQ ID NO: 118), FIG. 120 (SEQ ID NO: 120), FIG. 122 (SEQ ID NO: 122), FIG. 124 (SEQ ID NO: 124), FIG. 126 (SEQ ID NO: 126), FIG. 128 (SEQ ID NO: 128), FIG. 130 (SEQ ID NO: 130), FIG. 132 (SEQ ID NO: 132), FIG. 134 (SEQ ID NO: 134), FIG. 136 (SEQ ID NO: 136), FIG. 138 (SEQ ID NO: 138), FIG. 140 (SEQ ID NO: 140), FIG. 142 (SEQ ID NO: 142), FIG. 144 (SEQ ID NO: 144), FIG. 146 (SEQ ID NO: 146), FIG. 148 (SEQ ID NO: 148), FIG. 150 (SEQ ID NO: 150), FIG. 152 (SEQ ID NO: 152), FIG. 154 (SEQ ID NO: 154), FIG. 156 (SEQ ID NO: 156), FIG. 158 (SEQ ID NO: 158), FIG. 160 (SEQ ID NO: 160), FIG. 162 (SEQ ID NO: 162), FIG. 164 (SEQ ID NO: 164), FIG. 166 (SEQ ID NO: 166), FIG. 168 (SEQ ID NO: 168), FIG. 170 (SEQ ID NO: 170), FIG. 172 (SEQ ID NO: 172), FIG. 174 (SEQ ID NO: 174), FIG. 176 (SEQ ID NO: 176), FIG. 178 (SEQ ID NO: 178), FIG. 180 (SEQ ID NO: 180), FIG. 182 (SEQ ID NO: 182), FIG. 184 (SEQ ID NO: 184), FIG. 186 (SEQ ID NO: 186), FIG. 188 (SEQ ID NO: 188), FIG. 190 (SEQ ID NO: 190), FIG. 192 (SEQ ID NO: 192), FIG. 194 (SEQ ID NO: 194), FIG. 196 (SEQ ID NO: 196), FIG. 198 (SEQ ID NO: 198), FIG. 200 (SEQ ID NO: 200), FIG. 202 (SEQ ID NO: 202), FIG. 204 (SEQ ID NO: 204), FIG. 206 (SEQ ID NO: 206), FIG. 208 (SEQ ID NO: 208), FIG. 210 (SEQ ID NO: 210), FIG. 212 (SEQ ID NO: 212), FIG. 214 (SEQ ID NO: 214), FIG. 216 (SEQ ID NO: 216), FIG. 218 (SEQ ID NO: 218), FIG. 220 (SEQ ID NO: 220), FIG. 222 (SEQ ID NO: 222), FIG. 224 (SEQ ID NO: 224), FIG. 226 (SEQ ID NO: 226), FIG. 228 (SEQ ID NO: 228), FIG. 230 (SEQ ID NO: 230), FIG. 232 (SEQ ID NO: 232), FIG. 234 (SEQ ID NO: 234), FIG. 236 (SEQ ID NO: 236), FIG. 238 (SEQ ID NO: 238), FIG. 240 (SEQ ID NO: 240), FIG. 242 (SEQ ID NO: 242), FIG. 244 (SEQ ID NO: 244), FIG. 246 (SEQ ID NO: 246), FIG. 248 (SEQ ID NO: 248), FIG. 250 (SEQ ID NO: 250), FIG. 252 (SEQ ID NO: 252), FIG. 254 (SEQ ID NO: 254), FIG. 256 (SEQ ID NO: 256), FIG. 258 (SEQ ID NO: 258), FIG. 260 (SEQ ID NO: 260), FIG. 262 (SEQ ID NO: 262), FIG. 264 (SEQ ID NO: 264), FIG. 266 (SEQ ID NO: 266), FIG. 268 (SEQ ID NO: 268), FIG. 270 (SEQ ID NO: 270), FIG. 272 (SEQ ID NO: 272), FIG. 274 (SEQ ID NO: 274), FIG. 276 (SEQ ID NO: 276), FIG. 278 (SEQ ID NO: 278), FIG. 280 (SEQ ID NO: 280), FIG. 282 (SEQ ID NO: 282), FIG. 284 (SEQ ID NO: 284), FIG. 286 (SEQ ID NO: 286), FIG. 288 (SEQ ID NO: 288), FIG. 290 (SEQ ID NO: 290), FIG. 292 (SEQ ID NO: 292), FIG. 294 (SEQ ID NO: 294), FIG. 296 (SEQ ID NO: 296), FIG. 298 (SEQ ID NO: 298), FIG. 300 (SEQ ID NO: 300), FIG. 302 (SEQ ID NO: 302), FIG. 304 (SEQ ID NO: 304), FIG. 306 (SEQ ID NO: 306), FIG. 308 (SEQ ID NO: 308), FIG. 310 (SEQ ID NO: 310), FIG. 312 (SEQ ID NO: 312), FIG. 314 (SEQ ID NO: 314), FIG. 316 (SEQ ID NO: 316), FIG. 318 (SEQ ID NO: 318), FIG. 320 (SEQ ID NO: 320), FIG. 322 (SEQ ID NO: 322), FIG. 324 (SEQ ID NO: 324), FIG. 326 (SEQ ID NO: 326), FIG. 328 (SEQ ID NO: 328), FIG. 330 (SEQ ID NO: 330), FIG. 332 (SEQ ID NO: 332), FIG. 334 (SEQ ID NO: 334), FIG. 336 (SEQ ID NO: 336), FIG. 338 (SEQ ID NO: 338), FIG. 340 (SEQ ID NO: 340), FIG. 342 (SEQ ID NO: 342), FIG. 344 (SEQ ID NO: 344), FIG. 346 (SEQ ID NO: 346), FIG. 348 (SEQ ID NO: 348), FIG. 350 (SEQ ID NO: 350), FIG. 352 (SEQ ID NO: 352), FIG. 354 (SEQ ID NO: 354), FIG. 356 (SEQ ID NO: 356), FIG. 358 (SEQ ID NO: 358), FIG. 360 (SEQ ID NO: 360), FIG. 362 (SEQ ID NO: 362), FIG. 364 (SEQ ID NO: 364), FIG. 366 (SEQ ID NO: 366), FIG. 368 (SEQ ID NO: 368), FIG. 370 (SEQ ID NO: 370), FIG. 372 (SEQ ID NO: 372), FIG. 374 (SEQ ID NO: 374), FIG. 376 (SEQ ID NO: 376), FIG. 378 (SEQ ID NO: 378), FIG. 380 (SEQ ID NO: 380), FIG. 382 (SEQ ID NO: 382), FIG. 384 (SEQ ID NO: 384), FIG. 386 (SEQ ID NO: 386), FIG. 388 (SEQ ID NO: 388), FIG. 390 (SEQ ID NO: 390), FIG. 392 (SEQ ID NO: 392), FIG. 394 (SEQ ID NO: 394), FIG. 396 (SEQ ID NO: 396), FIG. 398 (SEQ ID NO: 398), FIG. 400 (SEQ ID NO: 400), FIG. 402 (SEQ ID NO: 402), FIG. 404 (SEQ ID NO: 404), FIG. 406 (SEQ ID NO: 406), FIG. 408 (SEQ ID NO: 408), FIG. 410 (SEQ ID NO: 410), FIG. 412 (SEQ ID NO: 412), FIG. 414 (SEQ ID NO: 414), FIG. 416 (SEQ ID NO: 416), FIG. 418 (SEQ ID NO: 418), FIG. 420 (SEQ ID NO: 420), FIG. 422 (SEQ ID NO: 422), FIG. 424 (SEQ ID NO: 424), FIG. 426 (SEQ ID NO: 426), FIG. 428 (SEQ ID NO: 428), FIG. 430 (SEQ ID NO: 430), FIG. 432 (SEQ ID NO: 432), FIG. 434 (SEQ ID NO: 434), FIG. 436 (SEQ ID NO: 436), FIG. 438 (SEQ ID NO: 438), FIG. 440 (SEQ ID NO: 440), FIG. 442 (SEQ ID NO: 442), FIG. 444 (SEQ ID NO: 444), FIG. 446 (SEQ ID NO: 446), FIG. 448 (SEQ ID NO: 448), FIG. 450 (SEQ ID NO: 450), FIG. 452 (SEQ ID NO: 452), FIG. 454 (SEQ ID NO: 454), FIG. 456 (SEQ ID NO: 456), FIG. 458 (SEQ ID NO: 458), FIG. 460 (SEQ ID NO: 460), FIG. 462 (SEQ ID NO: 462), FIG. 464 (SEQ ID NO: 464), FIG. 466 (SEQ ID NO: 466), FIG. 468 (SEQ ID NO: 468), FIG. 470 (SEQ ID NO: 470), FIG. 472 (SEQ ID NO: 472), FIG. 474 (SEQ ID NO: 474), FIG. 476 (SEQ ID NO: 476), FIG. 478 (SEQ ID NO: 478), FIG. 480 (SEQ ID NO: 480), FIG. 482 (SEQ ID NO: 482), FIG. 484 (SEQ ID NO: 484), FIG. 486 (SEQ ID NO: 486), FIG. 488 (SEQ ID NO: 488), FIG. 490 (SEQ ID NO: 490), FIG. 492 (SEQ ID NO: 492), FIG. 494 (SEQ ID NO: 494), FIG. 496 (SEQ ID NO: 496), FIG. 498 (SEQ ID NO: 498), FIG. 500 (SEQ ID NO: 500), FIG. 502 (SEQ ID NO: 502), FIG. 504 (SEQ ID NO: 504), FIG. 506 (SEQ ID NO: 506), FIG. 508 (SEQ ID NO: 508), FIG. 510 (SEQ ID NO: 510), FIG. 512 (SEQ ID NO: 512), FIG. 514 (SEQ ID NO: 514), FIG. 516 (SEQ ID NO: 516), FIG. 518 (SEQ ID NO: 518), FIG. 520 (SEQ ID NO: 520), FIG. 522 (SEQ ID NO: 522), FIG. 524 (SEQ ID NO: 524), FIG. 526 (SEQ ID NO: 526), FIG. 528 (SEQ ID NO: 528), FIG. 530 (SEQ ID NO: 530), FIG. 532 (SEQ ID NO: 532), FIG. 534 (SEQ ID NO: 534), FIG. 536 (SEQ ID NO: 536), FIG. 538 (SEQ ID NO: 538), FIG. 540 (SEQ ID NO: 540), FIG. 542 (SEQ ID NO: 542), FIG. 544 (SEQ ID NO: 544), FIG. 546 (SEQ ID NO: 546), FIG. 548 (SEQ ID NO: 548), FIG. 550 (SEQ ID NO: 550), FIG. 552 (SEQ ID NO: 552), FIG. 554 (SEQ ID NO: 554), FIG. 556 (SEQ ID NO: 556), FIG. 558 (SEQ ID NO: 558), FIG. 560 (SEQ ID NO: 560), FIG. 562 (SEQ ID NO: 562), FIG. 564 (SEQ ID NO: 564), FIG. 566 (SEQ ID NO: 566), FIG. 568 (SEQ ID NO: 568), FIG. 570 (SEQ ID NO: 570), FIG. 572 (SEQ ID NO: 572), FIG. 574 (SEQ ID NO: 574), FIG. 576 (SEQ ID NO: 576), FIG. 578 (SEQ ID NO: 578), FIG. 580 (SEQ ID NO: 580), FIG. 582 (SEQ ID NO: 582), FIG. 584 (SEQ ID NO: 584), FIG. 586 (SEQ ID NO: 586), FIG. 588 (SEQ ID NO: 588), FIG. 590 (SEQ ID NO: 590), FIG. 592 (SEQ ID NO: 592), FIG. 594 (SEQ ID NO: 594), FIG. 596 (SEQ ID NO: 596), FIG. 598 (SEQ ID NO: 598), FIG. 600 (SEQ ID NO: 600), FIG. 602 (SEQ ID NO: 602), FIG. 604 (SEQ ID NO: 604), FIG. 606 (SEQ ID NO: 606), FIG. 608 (SEQ ID NO: 608), or FIG. 610 (SEQ ID NO: 610), lacking its associated signal peptide;
(b) an amino acid sequence of an extracellular domain of the polypeptide shown in FIG. 2 (SEQ ID NO: 2), FIG. 4 (SEQ ID NO: 4), FIG. 6 (SEQ ID NO: 6), FIG. 8 (SEQ ID NO: 8), FIG. 10 (SEQ ID NO: 10), FIG. 12 (SEQ ID NO: 12), FIG. 14 (SEQ ID NO: 14), FIG. 16 (SEQ ID NO: 16), FIG. 18 (SEQ ID NO: 18), FIG. 20 (SEQ ID NO: 20), FIG. 22 (SEQ ID NO: 22), FIG. 24 (SEQ ID NO: 24), FIG. 26 (SEQ ID NO: 26), FIG. 28 (SEQ ID NO: 28), FIG. 30 (SEQ ID NO: 30), FIG. 32 (SEQ ID NO: 32), FIG. 34 (SEQ ID NO: 34), FIG. 36 (SEQ ID NO: 36), FIG. 38 (SEQ ID NO: 38), FIG. 40 (SEQ ID NO: 40), FIG. 42 (SEQ ID NO: 42), FIG. 44 (SEQ ID NO: 44), FIG. 46 (SEQ ID NO: 46), FIG. 48 (SEQ ID NO: 48), FIG. 50 (SEQ ID NO: 50), FIG. 52 (SEQ ID NO: 52), FIG. 54 (SEQ ID NO: 54), FIG. 56 (SEQ ID NO: 56), FIG. 58 (SEQ ID NO: 58), FIG. 60 (SEQ ID NO: 60), FIG. 62 (SEQ ID NO: 62), FIG. 64 (SEQ ID NO: 64), FIG. 66 (SEQ ID NO: 66), FIG. 68 (SEQ ID NO: 68), FIG. 70 (SEQ ID NO: 70), FIG. 72 (SEQ ID NO: 72), FIG. 74 (SEQ ID NO: 74), FIG. 76 (SEQ ID NO: 76), FIG. 78 (SEQ ID NO: 78), FIG. 80 (SEQ ID NO: 80), FIG. 82 (SEQ ID NO: 82), FIG. 84 (SEQ ID NO: 84), FIG. 86 (SEQ ID NO: 86), FIG. 88 (SEQ ID NO: 88), FIG. 90 (SEQ ID NO: 90), FIG. 92 (SEQ ID NO: 92), FIG. 94 (SEQ ID NO: 94), FIG. 96 (SEQ ID NO: 96), FIG. 98 (SEQ ID NO: 98), FIG. 100 (SEQ ID NO: 100), FIG. 102 (SEQ ID NO: 102), FIG. 104 (SEQ ID NO: 104), FIG. 106 (SEQ ID NO: 106), FIG. 108 (SEQ ID NO: 108), FIG. 110 (SEQ ID NO: 110), FIG. 112 (SEQ ID NO: 112), FIG. 114 (SEQ ID NO: 114), FIG. 116 (SEQ ID NO: 116), FIG. 118 (SEQ ID NO: 118), FIG. 120 (SEQ ID NO: 120), FIG. 122 (SEQ ID NO: 122), FIG. 124 (SEQ ID NO: 124), FIG. 126 (SEQ ID NO: 126), FIG. 128 (SEQ ID NO: 128), FIG. 130 (SEQ ID NO: 130), FIG. 132 (SEQ ID NO: 132), FIG. 134 (SEQ ID NO: 134), FIG. 136 (SEQ ID NO: 136), FIG. 138 (SEQ ID NO: 138), FIG. 140 (SEQ ID NO: 140), FIG. 142 (SEQ ID NO: 142), FIG. 144 (SEQ ID NO: 144), FIG. 146 (SEQ ID NO: 146), FIG. 148 (SEQ ID NO: 148), FIG. 150 (SEQ ID NO: 150), FIG. 152 (SEQ ID NO: 152), FIG. 154 (SEQ ID NO: 154), FIG. 156 (SEQ ID NO: 156), FIG. 158 (SEQ ID NO: 158), FIG. 160 (SEQ ID NO: 160), FIG. 162 (SEQ ID NO: 162), FIG. 164 (SEQ ID NO: 164), FIG. 166 (SEQ ID NO: 166), FIG. 168 (SEQ ID NO: 168), FIG. 170 (SEQ ID NO: 170), FIG. 172 (SEQ ID NO: 172), FIG. 174 (SEQ ID NO: 174), FIG. 176 (SEQ ID NO: 176), FIG. 178 (SEQ ID NO: 178), FIG. 180 (SEQ ID NO: 180), FIG. 182 (SEQ ID NO: 182), FIG. 184 (SEQ ID NO: 184), FIG. 186 (SEQ ID NO: 186), FIG. 188 (SEQ ID NO: 188), FIG. 190 (SEQ ID NO: 190), FIG. 192 (SEQ ID NO: 192), FIG. 194 (SEQ ID NO: 194), FIG. 196 (SEQ ID NO: 196), FIG. 198 (SEQ ID NO: 198), FIG. 200 (SEQ ID NO: 200), FIG. 202 (SEQ ID NO: 202), FIG. 204 (SEQ ID NO: 204), FIG. 206 (SEQ ID NO: 206), FIG. 208 (SEQ ID NO: 208), FIG. 210 (SEQ ID NO: 210), FIG. 212 (SEQ ID NO: 212), FIG. 214 (SEQ ID NO: 214), FIG. 216 (SEQ ID NO: 216), FIG. 218 (SEQ ID NO: 218), FIG. 220 (SEQ ID NO: 220), FIG. 222 (SEQ ID NO: 222), FIG. 224 (SEQ ID NO: 224), FIG. 226 (SEQ ID NO: 226), FIG. 228 (SEQ ID NO: 228), FIG. 230 (SEQ ID NO: 230), FIG. 232 (SEQ ID NO: 232), FIG. 234 (SEQ ID NO: 234), FIG. 236 (SEQ ID NO: 236), FIG. 238 (SEQ ID NO: 238), FIG. 240 (SEQ ID NO: 240), FIG. 242 (SEQ ID NO: 242), FIG. 244 (SEQ ID NO: 244), FIG. 246 (SEQ ID NO: 246), FIG. 248 (SEQ ID NO: 248), FIG. 250 (SEQ ID NO: 250), FIG. 252 (SEQ ID NO: 252), FIG. 254 (SEQ ID NO: 254), FIG. 256 (SEQ ID NO: 256), FIG. 258 (SEQ ID NO: 258), FIG. 260 (SEQ ID NO: 260), FIG. 262 (SEQ ID NO: 262), FIG. 264 (SEQ ID NO: 264), FIG. 266 (SEQ ID NO: 266), FIG. 268 (SEQ ID NO: 268), FIG. 270 (SEQ ID NO: 270), FIG. 272 (SEQ ID NO: 272), FIG. 274 (SEQ ID NO: 274), FIG. 276 (SEQ ID NO: 276), FIG. 278 (SEQ ID NO: 278), FIG. 280 (SEQ ID NO: 280), FIG. 282 (SEQ ID NO: 282), FIG. 284 (SEQ ID NO: 284), FIG. 286 (SEQ ID NO: 286), FIG. 288 (SEQ ID NO: 288), FIG. 290 (SEQ ID NO: 290), FIG. 292 (SEQ ID NO: 292), FIG. 294 (SEQ ID NO: 294), FIG. 296 (SEQ ID NO: 296), FIG. 298 (SEQ ID NO: 298), FIG. 300 (SEQ ID NO: 300), FIG. 302 (SEQ ID NO: 302), FIG. 304 (SEQ ID NO: 304), FIG. 306 (SEQ ID NO: 306), FIG. 308 (SEQ ID NO: 308), FIG. 310 (SEQ ID NO: 310), FIG. 312 (SEQ ID NO: 312), FIG. 314 (SEQ ID NO: 314), FIG. 316 (SEQ ID NO: 316), FIG. 318 (SEQ ID NO: 318), FIG. 320 (SEQ ID NO: 320), FIG. 322 (SEQ ID NO: 322), FIG. 324 (SEQ ID NO: 324), FIG. 326 (SEQ ID NO: 326), FIG. 328 (SEQ ID NO: 328), FIG. 330 (SEQ ID NO: 330), FIG. 332 (SEQ ID NO: 332), FIG. 334 (SEQ ID NO: 334), FIG. 336 (SEQ ID NO: 336), FIG. 338 (SEQ ID NO: 338), FIG. 340 (SEQ ID NO: 340), FIG. 342 (SEQ ID NO: 342), FIG. 344 (SEQ ID NO: 344), FIG. 346 (SEQ ID NO: 346), FIG. 348 (SEQ ID NO: 348), FIG. 350 (SEQ ID NO: 350), FIG. 352 (SEQ ID NO: 352), FIG. 354 (SEQ ID NO: 354), FIG. 356 (SEQ ID NO: 356), FIG. 358 (SEQ ID NO: 358), FIG. 360 (SEQ ID NO: 360), FIG. 362 (SEQ ID NO: 362), FIG. 364 (SEQ ID NO: 364), FIG. 366 (SEQ ID NO: 366), FIG. 368 (SEQ ID NO: 368), FIG. 370 (SEQ ID NO: 370), FIG. 372 (SEQ ID NO: 372), FIG. 374 (SEQ ID NO: 374), FIG. 376 (SEQ ID NO: 376), FIG. 378 (SEQ ID NO: 378), FIG. 380 (SEQ ID NO: 380), FIG. 382 (SEQ ID NO: 382), FIG. 384 (SEQ ID NO: 384), FIG. 386 (SEQ ID NO: 386), FIG. 388 (SEQ ID NO: 388), FIG. 390 (SEQ ID NO: 390), FIG. 392 (SEQ ID NO: 392), FIG. 394 (SEQ ID NO: 394), FIG. 396 (SEQ ID NO: 396), FIG. 398 (SEQ ID NO: 398), FIG. 400 (SEQ ID NO: 400), FIG. 402 (SEQ ID NO: 402), FIG. 404 (SEQ ID NO: 404), FIG. 406 (SEQ ID NO: 406), FIG. 408 (SEQ ID NO: 408), FIG. 410 (SEQ ID NO: 410), FIG. 412 (SEQ ID NO: 412), FIG. 414 (SEQ ID NO: 414), FIG. 416 (SEQ ID NO: 416), FIG. 418 (SEQ ID NO: 418), FIG. 420 (SEQ ID NO: 420), FIG. 422 (SEQ ID NO: 422), FIG. 424 (SEQ ID NO: 424), FIG. 426 (SEQ ID NO: 426), FIG. 428 (SEQ ID NO: 428), FIG. 430 (SEQ ID NO: 430), FIG. 432 (SEQ ID NO: 432), FIG. 434 (SEQ ID NO: 434), FIG. 436 (SEQ ID NO: 436), FIG. 438 (SEQ ID NO: 438), FIG. 440 (SEQ ID NO: 440), FIG. 442 (SEQ ID NO: 442), FIG. 444 (SEQ ID NO: 444), FIG. 446 (SEQ ID NO: 446), FIG. 448 (SEQ ID NO: 448), FIG. 450 (SEQ ID NO: 450), FIG. 452 (SEQ ID NO: 452), FIG. 454 (SEQ ID NO: 454), FIG. 456 (SEQ ID NO: 456), FIG. 458 (SEQ ID NO: 458), FIG. 460 (SEQ ID NO: 460), FIG. 462 (SEQ ID NO: 462), FIG. 464 (SEQ ID NO: 464), FIG. 466 (SEQ ID NO: 466), FIG. 468 (SEQ ID NO: 468), FIG. 470 (SEQ ID NO: 470), FIG. 472 (SEQ ID NO: 472), FIG. 474 (SEQ ID NO: 474), FIG. 476 (SEQ ID NO: 476), FIG. 478 (SEQ ID NO: 478), FIG. 480 (SEQ ID NO: 480), FIG. 482 (SEQ ID NO: 482), FIG. 484 (SEQ ID NO: 484), FIG. 486 (SEQ ID NO: 486), FIG. 488 (SEQ ID NO: 488), FIG. 490 (SEQ ID NO: 490), FIG. 492 (SEQ ID NO: 492), FIG. 494 (SEQ ID NO: 494), FIG. 496 (SEQ ID NO: 496), FIG. 498 (SEQ ID NO: 498), FIG. 500 (SEQ ID NO: 500), FIG. 502 (SEQ ID NO: 502), FIG. 504 (SEQ ID NO: 504), FIG. 506 (SEQ ID NO: 506), FIG. 508 (SEQ ID NO: 508), FIG. 510 (SEQ ID NO: 510), FIG. 512 (SEQ ID NO: 512), FIG. 514 (SEQ ID NO: 514), FIG. 516 (SEQ ID NO: 516), FIG. 518 (SEQ ID NO: 518), FIG. 520 (SEQ ID NO: 520), FIG. 522 (SEQ ID NO: 522), FIG. 524 (SEQ ID NO: 524), FIG. 526 (SEQ ID NO: 526), FIG. 528 (SEQ ID NO: 528), FIG. 530 (SEQ ID NO: 530), FIG. 532 (SEQ ID NO: 532), FIG. 534 (SEQ ID NO: 534), FIG. 536 (SEQ ID NO: 536), FIG. 538 (SEQ ID NO: 538), FIG. 540 (SEQ ID NO: 540), FIG. 542 (SEQ ID NO: 542), FIG. 544 (SEQ ID NO: 544), FIG. 546 (SEQ ID NO: 546), FIG. 548 (SEQ ID NO: 548), FIG. 550 (SEQ ID NO: 550), FIG. 552 (SEQ ID NO: 552), FIG. 554 (SEQ ID NO: 554), FIG. 556 (SEQ ID NO: 556), FIG. 558 (SEQ ID NO: 558), FIG. 560 (SEQ ID NO: 560), FIG. 562 (SEQ ID NO: 562), FIG. 564 (SEQ ID NO: 564), FIG. 566 (SEQ ID NO: 566), FIG. 568 (SEQ ID NO: 568), FIG. 570 (SEQ ID NO: 570), FIG. 572 (SEQ ID NO: 572), FIG. 574 (SEQ ID NO: 574), FIG. 576 (SEQ ID NO: 576), FIG. 578 (SEQ ID NO: 578), FIG. 580 (SEQ ID NO: 580), FIG. 582 (SEQ ID NO: 582), FIG. 584 (SEQ ID NO: 584), FIG. 586 (SEQ ID NO: 586), FIG. 588 (SEQ ID NO: 588), FIG. 590 (SEQ ID NO: 590), FIG. 592 (SEQ ID NO: 592), FIG. 594 (SEQ ID NO: 594), FIG. 596 (SEQ ID NO: 596), FIG. 598 (SEQ ID NO: 598), FIG. 600 (SEQ ID NO: 600), FIG. 602 (SEQ ID NO: 602), FIG. 604 (SEQ ID NO: 604), FIG. 606 (SEQ ID NO: 606), FIG. 608 (SEQ ID NO: 608), or FIG. 610 (SEQ ID NO: 610), with its associated signal peptide; or
(c) an amino acid sequence of an extracellular domain of the polypeptide shown in FIG. 2 (SEQ ID NO: 2), FIG. 4 (SEQ ID NO: 4), FIG. 6 (SEQ ID NO: 6), FIG. 8 (SEQ ID NO: 8), FIG. 10 (SEQ ID NO: 10), FIG. 12 (SEQ ID NO: 12), FIG. 14 (SEQ ID NO: 14), FIG. 16 (SEQ ID NO: 16), FIG. 18 (SEQ ID NO: 18), FIG. 20 (SEQ ID NO: 20), FIG. 22 (SEQ ID NO: 22), FIG. 24 (SEQ ID NO: 24), FIG. 26 (SEQ ID NO: 26), FIG. 28 (SEQ ID NO: 28), FIG. 30 (SEQ ID NO: 30), FIG. 32 (SEQ ID NO: 32), FIG. 34 (SEQ ID NO: 34), FIG. 36 (SEQ ID NO: 36), FIG. 38 (SEQ ID NO: 38), FIG. 40 (SEQ ID NO: 40), FIG. 42 (SEQ ID NO: 42), FIG. 44 (SEQ ID NO: 44), FIG. 46 (SEQ ID NO: 46), FIG. 48 (SEQ ID NO: 48), FIG. 50 (SEQ ID NO: 50), FIG. 52 (SEQ ID NO: 52), FIG. 54 (SEQ ID NO: 54), FIG. 56 (SEQ ID NO: 56), FIG. 58 (SEQ ID NO: 58), FIG. 60 (SEQ ID NO: 60), FIG. 62 (SEQ ID NO: 62), FIG. 64 (SEQ ID NO: 64), FIG. 66 (SEQ ID NO: 66), FIG. 68 (SEQ ID NO: 68), FIG. 70 (SEQ ID NO: 70), FIG. 72 (SEQ ID NO: 72), FIG. 74 (SEQ ID NO: 74), FIG. 76 (SEQ ID NO: 76), FIG. 78 (SEQ ID NO: 78), FIG. 80 (SEQ ID NO: 80), FIG. 82 (SEQ ID NO: 82), FIG. 84 (SEQ ID NO: 84), FIG. 86 (SEQ ID NO: 86), FIG. 88 (SEQ ID NO: 88), FIG. 90 (SEQ ID NO: 90), FIG. 92 (SEQ ID NO: 92), FIG. 94 (SEQ ID NO: 94), FIG. 96 (SEQ ID NO: 96), FIG. 98 (SEQ ID NO: 98), FIG. 100 (SEQ ID NO: 100), FIG. 102 (SEQ ID NO: 102), FIG. 104 (SEQ ID NO: 104), FIG. 106 (SEQ ID NO: 106), FIG. 108 (SEQ ID NO: 108), FIG. 110 (SEQ ID NO: 110), FIG. 112 (SEQ ID NO: 112), FIG. 114 (SEQ ID NO: 114), FIG. 116 (SEQ ID NO: 116), FIG. 118 (SEQ ID NO: 118), FIG. 120 (SEQ ID NO: 120), FIG. 122 (SEQ ID NO: 122), FIG. 124 (SEQ ID NO: 124), FIG. 126 (SEQ ID NO: 126), FIG. 128 (SEQ ID NO: 128), FIG. 130 (SEQ ID NO: 130), FIG. 132 (SEQ ID NO: 132), FIG. 134 (SEQ ID NO: 134), FIG. 136 (SEQ ID NO: 136), FIG. 138 (SEQ ID NO: 138), FIG. 140 (SEQ ID NO: 140), FIG. 142 (SEQ ID NO: 142), FIG. 144 (SEQ ID NO: 144), FIG. 146 (SEQ ID NO: 146), FIG. 148 (SEQ ID NO: 148), FIG. 150 (SEQ ID NO: 150), FIG. 152 (SEQ ID NO: 152), FIG. 154 (SEQ ID NO: 154), FIG. 156 (SEQ ID NO: 156), FIG. 158 (SEQ ID NO: 158), FIG. 160 (SEQ ID NO: 160), FIG. 162 (SEQ ID NO: 162), FIG. 164 (SEQ ID NO: 164), FIG. 166 (SEQ ID NO: 166), FIG. 168 (SEQ ID NO: 168), FIG. 170 (SEQ ID NO: 170), FIG. 172 (SEQ ID NO: 172), FIG. 174 (SEQ ID NO: 174), FIG. 176 (SEQ ID NO: 176), FIG. 178 (SEQ ID NO: 178), FIG. 180 (SEQ ID NO: 180), FIG. 182 (SEQ ID NO: 182), FIG. 184 (SEQ ID NO: 184), FIG. 186 (SEQ ID NO: 186), FIG. 188 (SEQ ID NO: 188), FIG. 190 (SEQ ID NO: 190), FIG. 192 (SEQ ID NO: 192), FIG. 194 (SEQ ID NO: 194), FIG. 196 (SEQ ID NO: 196), FIG. 198 (SEQ ID NO: 198), FIG. 200 (SEQ ID NO: 200), FIG. 202 (SEQ ID NO: 202), FIG. 204 (SEQ ID NO: 204), FIG. 206 (SEQ ID NO: 206), FIG. 208 (SEQ ID NO: 208), FIG. 210 (SEQ ID NO: 210), FIG. 212 (SEQ ID NO: 212), FIG. 214 (SEQ ID NO: 214), FIG. 216 (SEQ ID NO: 216), FIG. 218 (SEQ ID NO: 218), FIG. 220 (SEQ ID NO: 220), FIG. 222 (SEQ ID NO: 222), FIG. 224 (SEQ ID NO: 224), FIG. 226 (SEQ ID NO: 226), FIG. 228 (SEQ ID NO: 228), FIG. 230 (SEQ ID NO: 230), FIG. 232 (SEQ ID NO: 232), FIG. 234 (SEQ ID NO: 234), FIG. 236 (SEQ ID NO: 236), FIG. 238 (SEQ ID NO: 238), FIG. 240 (SEQ ID NO: 240), FIG. 242 (SEQ ID NO: 242), FIG. 244 (SEQ ID NO: 244), FIG. 246 (SEQ ID NO: 246), FIG. 248 (SEQ ID NO: 248), FIG. 250 (SEQ ID NO: 250), FIG. 252 (SEQ ID NO: 252), FIG. 254 (SEQ ID NO: 254), FIG. 256 (SEQ ID NO: 256), FIG. 258 (SEQ ID NO: 258), FIG. 260 (SEQ ID NO: 260), FIG. 262 (SEQ ID NO: 262), FIG. 264 (SEQ ID NO: 264), FIG. 266 (SEQ ID NO: 266), FIG. 268 (SEQ ID NO: 268), FIG. 270 (SEQ ID NO: 270), FIG. 272 (SEQ ID NO: 272), FIG. 274 (SEQ ID NO: 274), FIG. 276 (SEQ ID NO: 276), FIG. 278 (SEQ ID NO: 278), FIG. 280 (SEQ ID NO: 280), FIG. 282 (SEQ ID NO: 282), FIG. 284 (SEQ ID NO: 284), FIG. 286 (SEQ ID NO: 286), FIG. 288 (SEQ ID NO: 288), FIG. 290 (SEQ ID NO: 290), FIG. 292 (SEQ ID NO: 292), FIG. 294 (SEQ ID NO: 294), FIG. 296 (SEQ ID NO: 296), FIG. 298 (SEQ ID NO: 298), FIG. 300 (SEQ ID NO: 300), FIG. 302 (SEQ ID NO: 302), FIG. 304 (SEQ ID NO: 304), FIG. 306 (SEQ ID NO: 306), FIG. 308 (SEQ ID NO: 308), FIG. 310 (SEQ ID NO: 310), FIG. 312 (SEQ ID NO: 312), FIG. 314 (SEQ ID NO: 314), FIG. 316 (SEQ ID NO: 316), FIG. 318 (SEQ ID NO: 318), FIG. 320 (SEQ ID NO: 320), FIG. 322 (SEQ ID NO: 322), FIG. 324 (SEQ ID NO: 324), FIG. 326 (SEQ ID NO: 326), FIG. 328 (SEQ ID NO: 328), FIG. 330 (SEQ ID NO: 330), FIG. 332 (SEQ ID NO: 332), FIG. 334 (SEQ ID NO: 334), FIG. 336 (SEQ ID NO: 336), FIG. 338 (SEQ ID NO: 338), FIG. 340 (SEQ ID NO: 340), FIG. 342 (SEQ ID NO: 342), FIG. 344 (SEQ ID NO: 344), FIG. 346 (SEQ ID NO: 346), FIG. 348 (SEQ ID NO: 348), FIG. 350 (SEQ ID NO: 350), FIG. 352 (SEQ ID NO: 352), FIG. 354 (SEQ ID NO: 354), FIG. 356 (SEQ ID NO: 356), FIG. 358 (SEQ ID NO: 358), FIG. 360 (SEQ ID NO: 360), FIG. 362 (SEQ ID NO: 362), FIG. 364 (SEQ ID NO: 364), FIG. 366 (SEQ ID NO: 366), FIG. 368 (SEQ ID NO: 368), FIG. 370 (SEQ ID NO: 370), FIG. 372 (SEQ ID NO: 372), FIG. 374 (SEQ ID NO: 374), FIG. 376 (SEQ ID NO: 376), FIG. 378 (SEQ ID NO: 378), FIG. 380 (SEQ ID NO: 380), FIG. 382 (SEQ ID NO: 382), FIG. 384 (SEQ ID NO: 384), FIG. 386 (SEQ ID NO: 386), FIG. 388 (SEQ ID NO: 388), FIG. 390 (SEQ ID NO: 390), FIG. 392 (SEQ ID NO: 392), FIG. 394 (SEQ ID NO: 394), FIG. 396 (SEQ ID NO: 396), FIG. 398 (SEQ ID NO: 398), FIG. 400 (SEQ ID NO: 400), FIG. 402 (SEQ ID NO: 402), FIG. 404 (SEQ ID NO: 404), FIG. 406 (SEQ ID NO: 406), FIG. 408 (SEQ ID NO: 408), FIG. 410 (SEQ ID NO: 410), FIG. 412 (SEQ ID NO: 412), FIG. 414 (SEQ ID NO: 414), FIG. 416 (SEQ ID NO: 416), FIG. 418 (SEQ ID NO: 418), FIG. 420 (SEQ ID NO: 420), FIG. 422 (SEQ ID NO: 422), FIG. 424 (SEQ ID NO: 424), FIG. 426 (SEQ ID NO : 426), FIG. 428 (SEQ ID NO: 428), FIG. 430 (SEQ ID NO: 430), FIG. 432 (SEQ ID NO: 432), FIG. 434 (SEQ ID NO: 434), FIG. 436 (SEQ ID NO: 436), FIG. 438 (SEQ ID NO: 438), FIG. 440 (SEQ ID NO: 440), FIG. 442 (SEQ ID NO: 442), FIG. 444 (SEQ ID NO: 444), FIG. 446 (SEQ ID NO: 446), FIG. 448 (SEQ ID NO: 448), FIG. 450 (SEQ ID NO: 450), FIG. 452 (SEQ ID NO: 452), FIG. 454 (SEQ ID NO: 454), FIG. 456 (SEQ ID NO: 456), FIG. 458 (SEQ ID NO: 458), FIG. 460 (SEQ ID NO: 460), FIG. 462 (SEQ ID NO: 462), FIG. 464 (SEQ ID NO: 464), FIG. 466 (SEQ ID NO: 466), FIG. 468 (SEQ ID NO: 468), FIG. 470 (SEQ ID NO: 470), FIG. 472 (SEQ ID NO: 472), FIG. 474 (SEQ ID NO: 474), FIG. 476 (SEQ ID NO: 476), FIG. 478 (SEQ ID NO: 478), FIG. 480 (SEQ ID NO: 480), FIG. 482 (SEQ ID NO: 482), FIG. 484 (SEQ ID NO: 484), FIG. 486 (SEQ ID NO: 486), FIG. 488 (SEQ ID NO: 488), FIG. 490 (SEQ ID NO: 490), FIG. 492 (SEQ ID NO: 492), FIG. 494 (SEQ ID NO: 494), FIG. 496 (SEQ ID NO: 496), FIG. 498 (SEQ ID NO: 498), FIG. 500 (SEQ ID NO: 500), FIG. 502 (SEQ ID NO: 502), FIG. 504 (SEQ ID NO: 504), FIG. 506 (SEQ ID NO: 506), FIG. 508 (SEQ ID NO: 508), FIG. 510 (SEQ ID NO: 510), FIG. 512 (SEQ ID NO: 512), FIG. 514 (SEQ ID NO: 514), FIG. 516 (SEQ ID NO: 516), FIG. 518 (SEQ ID NO: 518), FIG. 520 (SEQ ID NO: 520), FIG. 522 (SEQ ID NO: 522), FIG. 524 (SEQ ID NO: 524), FIG. 526 (SEQ ID NO: 526), FIG. 528 (SEQ ID NO: 528), FIG. 530 (SEQ ID NO: 530), FIG. 532 (SEQ ID NO: 532), FIG. 534 (SEQ ID NO: 534), FIG. 536 (SEQ ID NO: 536), FIG. 538 (SEQ ID NO: 538), FIG. 540 (SEQ ID NO: 540), FIG. 542 (SEQ ID NO: 542), FIG. 544 (SEQ ID NO: 544), FIG. 546 (SEQ ID NO: 546), FIG. 548 (SEQ ID NO: 548), FIG. 550 (SEQ ID NO: 550), FIG. 552 (SEQ ID NO: 552), FIG. 554 (SEQ ID NO: 554), FIG. 556 (SEQ ID NO: 556), FIG. 558 (SEQ ID NO: 558), FIG. 560 (SEQ ID NO: 560), FIG. 562 (SEQ ID NO: 562), FIG. 564 (SEQ ID NO: 564), FIG. 566 (SEQ ID NO: 566), FIG. 568 (SEQ ID NO: 568), FIG. 570 (SEQ ID NO: 570), FIG. 572 (SEQ ID NO: 572), FIG. 574 (SEQ ID NO: 574), FIG. 576 (SEQ ID NO: 576), FIG. 578 (SEQ ID NO: 578), FIG. 580 (SEQ ID NO: 580), FIG. 582 (SEQ ID NO: 582), FIG. 584 (SEQ ID NO: 584), FIG. 586 (SEQ ID NO: 586), FIG. 588 (SEQ ID NO: 588), FIG. 590 (SEQ ID NO: 590), FIG. 592 (SEQ ID NO: 592), FIG. 594 (SEQ ID NO: 594), FIG. 596 (SEQ ID NO: 596), FIG. 598 (SEQ ID NO: 598), FIG. 600 (SEQ ID NO: 600), FIG. 602 (SEQ ID NO: 602), FIG. 604 (SEQ ID NO: 604), FIG. 606 (SEQ ID NO: 606), FIG. 608 (SEQ ID NO: 608), or FIG. 610 (SEQ ID NO: 610), lacking its associated signal peptide.

20. A method for stimulating the release of TNF-α from human blood, said method comprising contacting said blood with a PRO1079, PRO827, PRO791, PRO1131, PRO1316, PRO1183, PRO1343, PRO1760, PRO1567 or PRO4333 polypeptide, wherein the release of TNF-α from said blood is stimulated.

21. A method for stimulating the proliferation or differentiation of chondrocyte cells, said method comprising contacting said cells with a PRO6029 polypeptide, wherein the proliferation or differentiation of said cells is stimulated.

22. A method for detecting the presence of tumor in an mammal, said method comprising comparing the level of expression of any PRO polypeptide shown in Table 8 in (a) a test sample of cells taken from said mammal and (b) a control sample of normal cells of the same cell type, wherein a higher level of expression of said PRO polypeptide in the test sample as compared to the control sample is indicative of the presence of tumor in said mammal.

23. The method of claim 22, wherein said tumor is adrenal tumor, lung tumor, colon tumor, breast tumor, prostate tumor, rectal tumor, cervical tumor or liver tumor.

24. An oligonucleotide probe derived from any of the nucleotide sequences shown in the accompanying figures.
